(19) United States
(12) Patent Application Publication
(10) Pub. No.: US 2025/0092375 A1
(43) Pub. Date: Mar. 20, 2025

(54) CAS ENDONUCLEASES AND RELATED METHODS

(71) Applicant: Flagship Pioneering Innovations VII, LLC, Cambridge, MA (US)

(72) Inventors: Molly Krisann GIBSON, Medford, MA (US); Pengfei TIAN, Boston, MA (US); Iain James MCFADYEN, Arlington, MA (US); Athanasios Dimitri DOUSIS, Somerville, MA (US); Jeffrey Ian BOUCHER, Cambridge, MA (US); Panagiota KYRIAKOU, Cambridge, MA (US); Pradeep RAMESH, Washington, DC (US)

(21) Appl. No.: 18/782,204

(22) Filed: Jul. 24, 2024

Related U.S. Application Data
(60) Provisional application No. 63/515,768, filed on Jul. 26, 2023.

(30) Foreign Application Priority Data
Jul. 25, 2023 (GR) 20230100610

(51) Int. Cl. C12N 9/22; C12N 15/11; C12N 15/90

(52) U.S. Cl. CPC C12N 9/22; C12N 15/11; C12N 15/907; C12N 2310/20

(57) ABSTRACT

Provided herein are Cas endonucleases (and functional fragments, functional variants, and domains thereof), nucleic acid molecules encoding the same, and systems comprising the same. The disclosure further relates to methods of utilizing the Cas endonucleases (or nucleic acid molecules encoding the same), including, e.g., in methods of editing a nucleic acid molecule (e.g., a gene) and methods of treating diseases (e.g., genetic diseases).

### RELATED APPLICATIONS

This application claims priority to Greek Patent Application No. 20230100610, filed Jul. 25, 2023; and U.S. Ser. No. 63/515,768, filed Jul. 26, 2023, the entire contents of each of which is incorporated herein by reference.

### SEQUENCE LISTING

The instant application contains a Sequence Listing which has been submitted electronically in XML format and is hereby incorporated by reference in its entirety. Said XML copy, created on Dec. 3, 2024, is named 62801_25US01_SL.xml and is 1,126,243 bytes in size.

### 1. FIELD

This disclosure relates to Cas endonucleases (and functional fragments, functional variants, and domains thereof), nucleic acid molecules encoding the same, and systems comprising the same. The disclosure further relates to methods of utilizing the Cas endonucleases (or nucleic acid molecules encoding the same), including, e.g., in methods of editing a nucleic acid molecule (e.g., a gene) and methods of treating diseases (e.g., genetic diseases).

### 2. BACKGROUND

CRISPR (clustered regularly interspaced short palindromic repeats)-Cas (CRISPR-associated protein) systems are adaptive immune systems of many prokaryotes (e.g., bacteria and archaea) that function to prevent infection (e.g., by phages, viruses, and other foreign genetic elements). Typical naturally occurring CRISPR-Cas systems comprise a CRISPR RNA (crRNA), a trans-activating CRISPR RNA (tracrRNA), and a Cas endonuclease, wherein the tracrRNA mediates binding to the Cas endonuclease, the crRNA directs the Cas endonuclease to a target nucleic acid molecule, and the Cas endonuclease mediates cleavage of the target nucleic acid molecule (e.g., viral DNA). CRISPR-Cas systems have been adapted and modified for nucleic acid (e.g., gene) editing in e.g., eukaryotic cells.

### 3. SUMMARY

Provided herein are, inter alia, novel Cas endonucleases and polynucleotides encoding the same; fusions and conjugates comprising a Cas endonuclease; methods of manufacturing; pharmaceutical compositions; and methods of use including, e.g., methods of editing a nucleic acid molecule (e.g., a gene) and methods of treating diseases (e.g., genetic diseases).

Accordingly, in one aspect provided herein are Cas endonucleases (or functional fragments, functional variants, or domains thereof) that comprises an amino acid sequence is at least 80%, 81%, 82% 83%, 84%, 85%, 86%, 87%, 88%, 89%, 90%, 91%, 92%, 93%, 94%, 95%, 96%, 97%, 98%, 99%, or 100% identical to the amino acid sequence of any Cas endonuclease set forth in Table 1 or set forth in any one of SEQ ID NOS: 1-320.

In some embodiments, the amino acid sequence is at least 85%, 86%, 87%, 88%, 89%, 90%, 91%, 92%, 93%, 94%, 95%, 96%, 97%, 98%, 99%, or 100% identical to the amino acid sequence of any Cas endonuclease set forth in Table 1 or set forth in any one of SEQ ID NOS: 1-320. In some embodiments, the amino acid sequence is at least 90%, 91%, 92%, 93%, 94%, 95%, 96%, 97%, 98%, 99%, or 100% identical to the amino acid sequence of any Cas endonuclease set forth in Table 1 or set forth in any one of SEQ ID NOS: 1-320.

In some embodiments, the amino acid sequence of the Cas endonuclease is less than 90%, 89%, 88%, 87%, 86%, 85%, 84%, 83%, 82%, 81%, 80%, 79%, 78%, 77%, 76%, or 75% identical to the amino acid sequence of a reference Cas endonuclease set forth in SEQ ID NO: 321. In some embodiments, the amino acid sequence of the Cas endonuclease is less than 90% (e.g., 89%, 88%, 87%, 86%, 85%, 84%, 83%, 82%, 81%, 80%, 79%, 78%, 77%, 76%, 75%, 74%, 73%, 72%, 71%, 60%, 69%, 68%, 67%, 66%, 65%, 64%, 63%, 62%, 61%, 60%, 59%, 58%, 57%, 56%, 55%, 54%, 53%, 52%, 51%) and greater than 50% (e.g., 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%) identical to the amino acid sequence of a reference Cas endonuclease set forth in SEQ ID NO: 321. In some embodiments, the amino acid sequence of the Cas endonuclease is less than 90% (e.g., 89%, 88%, 87%, 86%, 85%, 84%, 83%, 82%, 81%, 80%, 79%, 78%) and greater than 76% (e.g., 77%, 78%, 79%, 80%, 81%, 82%, 83%, 84%, 85%, 86%, 87%, 88%, 89%) identical to the amino acid sequence of a reference Cas endonuclease set forth in SEQ ID NO: 321.

In some embodiments, the Cas endonuclease has one or more (e.g., 1, 2, 3, 4, 5, and/or 6) of the following properties (or engineered to have one or more of the following properties): (a) the ability to mediate double strand breaks in a target double stranded nucleic acid (e.g., DNA) molecule; (b) the ability to mediate single strand breaks in a target double stranded nucleic acid (e.g., DNA) molecule; (c) the inability to mediate double strand breaks in a target double stranded nucleic acid (e.g., DNA) molecule; (d) the ability to mediate single strand breaks in a target double stranded nucleic acid (e.g., DNA) molecule and the inability to mediate double strand breaks in a target double stranded nucleic acid (e.g., DNA) molecule (i.e., nickase activity); (f) DNA endonuclease activity; and/or (g) RNA guided DNA endonuclease activity.

In some embodiments, the amino acid sequence of the Cas endonuclease comprises one or more amino acid variation (e.g., substitution, deletion, addition). In some embodiments, the one or more amino acid variation (e.g., substitution, deletion, addition) reduces or eliminates the ability of the Cas endonuclease to mediate double strand breaks in a target double stranded nucleic acid (e.g., DNA) molecule. In some embodiments, a modified Cas endonuclease comprising the one or more amino acid variation (e.g., substitution, deletion, addition) has the ability to mediate single strand breaks in a target double stranded nucleic acid (e.g., DNA) molecule) and does not have the ability to mediate double strand breaks in a target double stranded nucleic acid (e.g., DNA) molecule (i.e., nickase activity). In some embodiments, the one or more amino acid variation (e.g., substitution, deletion, addition) alters the PAM nucleotide sequence recognized by the Cas endonuclease. In some embodiments, the one or more amino acid variation (e.g., substitution, deletion, addition) (a) reduces the Cas endonuclease activity of the endonuclease by at least 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% relative to the endonuclease lacking the one or more amino acid variation (e.g., substitution, deletion, addition); or (b) enhances the Cas endonuclease activity of the endonuclease by at least 1-fold, 2-fold, 5-fold, 10-fold, or 100-fold relative to the Cas endonuclease lacking the one or more amino acid variation (e.g., substitution, deletion, addition).

In some embodiments, the Cas endonuclease further comprises one or more heterologous moiety (e.g., a heterologous protein). In some embodiments, the Cas endonuclease comprises 2, 3, 4, or 5 or more heterologous moieties. In some embodiments, the heterologous moiety is attached to the N-terminus, C-terminus, and/or internally between the N- and C-terminus of the endonuclease. In some embodiments, the heterologous moiety (e.g., heterologous protein) is directly attached to the endonuclease. In some embodiments, the heterologous moiety (e.g., heterologous protein) is indirectly attached to the Cas endonuclease. In some embodiments, the heterologous moiety (e.g., heterologous protein) is indirectly attached to the Cas endonuclease via a linker. In some embodiments, the heterologous moiety is a peptide, protein, carbohydrate, lipid, polymer, or small molecule. In some embodiments, the heterologous moiety is a nuclear localization signal (NLS), a tag, and/or a reporter gene.

In one aspect, provided herein are conjugates comprising a Cas endonuclease described herein and one or more heterologous moieties.

In some embodiments, the heterologous moiety is a protein, peptide, small molecule, nucleic acid molecule (e.g., DNA, RNA, DNA/RNA hybrid molecule), carbohydrate, lipid, or synthetic polymer. In some embodiments, the heterologous moiety is operably connected to the N-terminus, C-terminus, and/or internally between the N- and C-terminus of the Cas endonuclease. In some embodiments, the heterologous moiety is directly operably connected to the Cas endonuclease. In some embodiments, the heterologous moiety is indirectly operably connected to the Cas endonuclease. In some embodiments, the heterologous moiety is indirectly operably connected to the Cas endonuclease via a linker.

In one aspect, provided herein are fusion proteins comprising a Cas endonuclease described herein and one or more heterologous protein. In some embodiments, the heterologous protein is fused to the N-terminus, C-terminus, and/or internally between the N- and C-terminus of the Cas endonuclease. In some embodiments, the heterologous protein is fused directly to the Cas endonuclease. In some embodiments, the heterologous protein is fused indirectly to the Cas endonuclease. In some embodiments, the heterologous protein is fused indirectly to the Cas endonuclease via a peptide linker. In some embodiments, the heterologous protein exhibits polymerase (e.g., reverse transcriptase) activity, nucleobase editing activity (e.g., deaminase activity), methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, or double-strand DNA cleavage activity and nucleic acid binding activity, or any combination of the foregoing.

In some embodiments, the heterologous protein is a polymerase. In some embodiments, the polymerase has RNA-dependent DNA polymerase activity. In some embodiments, the polymerase is a reverse transcriptase (or a functional fragment, functional variant, or domain thereof). In some embodiments, the reverse transcriptase (or the functional fragment, functional variant, or domain thereof) is derived from a retrovirus or a retrotransposon. In some embodiments, the reverse transcriptase (or the functional fragment, functional variant, or domain thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 2 or set forth in any one of SEQ ID NOS: 324-476.

In some embodiments, the heterologous polypeptide is a nucleobase editor. In some embodiments, the nucleobase editor is a deaminase (or a functional fragment, functional variant, or domain thereof). In some embodiments, the deaminase (or the functional fragment, functional variant, or domain thereof) exhibits adenosine deaminase activity and/or a or a cytidine deaminase activity. In some embodiments, the deaminase (or a functional fragment, functional variant, or domain thereof) comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 3 or set forth in any one of SEQ ID NOS: 477-536. In some embodiments, the nucleobase editor is fused to an inhibitor of base excision repair (or a functional fragment or functional variant thereof) (e.g., uracil glycosylase inhibitor (UGI), nuclease dead inosine specific nuclease (dISN)).

In one aspect, provided herein are nucleic acid molecules encoding a Cas endonuclease described herein, a conjugate described herein, or a fusion protein described herein. In some embodiments, the nucleic acid molecule is a DNA or RNA (e.g., mRNA) molecule. In some embodiments, the nucleic acid molecule is codon optimized. In some embodiments, the nucleic acid molecule further comprises one or more transcription or translation regulatory elements (e.g., promoter, enhancer (e.g., cell or tissue specific transcription regulatory elements). In some embodiments, the nucleic acid molecule further encodes one or more gRNA (e.g., a crRNA, a tracrRNA, a sgRNA, a template RNA (e.g., as described herein)).

In one aspect, provided herein are vectors comprising a nucleic acid molecule described herein. In some embodiments, the vector is a viral vector or a non-viral vector (e.g., plasmid, minicircle). In some embodiments, the vector is a viral vector (e.g., an adeno associated viral (AAV) vector, a lentiviral vector, an adenoviral vector).

In one aspect, provided herein are carriers comprising a Cas endonuclease described herein, a conjugate described herein, a fusion protein described herein, a nucleic acid molecule described herein, and/or a vector described herein. In some embodiments, the carrier is a nanoparticle, polymer, virus (e.g., a recombinant virus), virus like particle, virosome, fusosome, vesicle, or lipid-based carrier. In some embodiments, the carrier is a recombinant virus (e.g., an adeno associated virus (AAV), a lentivirus, an adenovirus). In some embodiments, the carrier is a lipid-based carrier. In some embodiments, the lipid-based carrier is a lipid nanoparticle (LNP), liposome, lipoplex, nanoliposome, an exosome, or a micelle. In some embodiments, the carrier further comprises one or more gRNA (e.g., a crRNA, a tracrRNA, a sgRNA, a template RNA (e.g., as described herein)).

In one aspect, provided herein are reaction mixtures comprising (a) a cell (e.g., comprising a target nucleic acid molecule) or a target nucleic acid molecule; and (b) a Cas endonuclease described herein, a conjugate described herein, a fusion protein described herein, a nucleic acid molecule described herein, a vector described herein, a carrier described herein, and/or a pharmaceutical composition described herein.

In one aspect, provided herein are cells comprising a Cas endonuclease described herein, a conjugate described herein, a fusion protein described herein, a nucleic acid molecule described herein, a vector described herein, a reaction mixture described herein, a carrier described herein, and/or a pharmaceutical composition described herein.

In one aspect, provided herein are pharmaceutical compositions comprising a Cas endonuclease described herein, a conjugate described herein, a fusion protein described herein, a nucleic acid molecule described herein, a vector described herein, a reaction mixture described herein, a carrier described herein, and/or a cell described herein; and a pharmaceutically acceptable excipient.

In one aspect, provided herein are kits comprising a Cas endonuclease described herein, a conjugate described herein, a fusion protein described herein, a nucleic acid molecule described herein, a vector described herein, a reaction mixture described herein, a carrier described herein, a cell described herein, and/or a pharmaceutical composition described herein; and optionally instructions for using any one or more of the foregoing.

In one aspect, provided herein are systems for modifying a target nucleic acid (e.g., DNA) molecule, comprising: (a) a Cas endonuclease described herein, a conjugate described herein, a fusion protein described herein, a nucleic acid molecule described herein, a vector described herein, a carrier described herein, a reaction mixture described herein, a cell described herein, and/or a pharmaceutical composition described herein, and (b) a first gRNA (e.g., a crRNA and a tracrRNA; a sgRNA; a pegRNA, a template RNA (e.g., as described herein)) or a nucleic acid (e.g., DNA) molecule encoding the first gRNA (e.g., a crRNA and a tracrRNA; a sgRNA; template RNA (e.g., as described herein)).

In some embodiments, the system has one or more of the following characteristics: (a) the Cas endonuclease of the system is capable of binding to the first gRNA; (b) the Cas endonuclease of the system is capable of forming a break in a target nucleic acid (e.g., DNA (e.g., dsDNA)) molecule; (c) the Cas endonuclease of the system is capable of forming a single strand break in a target double stranded nucleic acid (e.g., DNA (e.g., dsDNA)) molecule; (d) the Cas endonuclease of the system is capable of forming a single strand break in the modified strand (as defined herein) of a target double stranded nucleic acid (e.g., DNA (e.g., dsDNA)) molecule; (e) the Cas endonuclease of the system is capable of forming a double strand break in a target double stranded nucleic acid (e.g., DNA (e.g., dsDNA)) molecule; (f) the Cas endonuclease of the system is incapable of forming a double strand break in a target double stranded nucleic acid (e.g., DNA (e.g., dsDNA)) molecule; (g) the Cas endonuclease of the system is capable of forming a single strand break in a target double stranded nucleic acid (e.g., DNA (e.g., dsDNA)) molecule and is incapable of forming a double strand break in a target double stranded nucleic acid (e.g., DNA (e.g., dsDNA)) molecule; (h) the Cas endonuclease of the system is capable of forming a single strand break in in the modified strand (as defined herein) of a target double stranded nucleic acid (e.g., DNA (e.g., dsDNA)) molecule and is incapable of forming a double strand break in a target double stranded nucleic acid (e.g., DNA (e.g., dsDNA)) molecule; and/or (i) the system is capable of editing (e.g., mediating the addition, deletion, or substitution of one or more nucleotides into/from) a target nucleic acid (e.g., DNA) molecule (e.g., a target double stranded DNA molecule).

In some embodiments, the system is capable of editing (e.g., mediating the addition, deletion, or substitution of one or more nucleotides into/from) a target nucleic acid (e.g., DNA) molecule (e.g., a target double stranded DNA molecule).

In some embodiments, the system is capable of editing (e.g., mediating the addition, deletion, or substitution of one or more nucleotides into/from) a target nucleic acid (e.g., DNA) molecule (e.g., a target double stranded DNA molecule) with increased efficiency relative to a reference system (e.g., comprising a reference Cas endonuclease (e.g., the reference Cas endonuclease set forth in SEQ ID NO: 321)).

In some embodiments, the system is capable of editing (e.g., mediating the addition, deletion, or substitution of one or more nucleotides into/from) a target nucleic acid (e.g., DNA) molecule (e.g., a target double stranded DNA molecule) with at least about a 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200% increase in efficiency relative to a reference system (e.g., comprising a reference Cas endonuclease) (e.g., the reference Cas endonuclease set forth in SEQ ID NO: 321)).

In some embodiments, the system is capable of editing (e.g., mediating the addition, deletion, or substitution of one or more nucleotides into/from) a target nucleic acid (e.g., DNA) molecule (e.g., a target double stranded DNA molecule) with at least about a 50%, 55%, 60%, 65%, 70%, 75%, 80%, 85%, 90%, 95%, 100% increase in efficiency relative to a reference system (e.g., comprising a reference Cas endonuclease) (e.g., the reference Cas endonuclease set forth in SEQ ID NO: 321)).

In some embodiments, the system is capable of editing (e.g., mediating the addition, deletion, or substitution of one or more nucleotides into/from) a target nucleic acid (e.g., DNA) molecule (e.g., a target double stranded DNA molecule) with from about a 30%-200%, 40%-200%, 50%-200%, 60%-200%, 70%-200%, 80%-200%, 90%-200%, 100%-200%, 150%-200%, 30%-150%, 40%-150%, 50%-150%, 60%-150%, 70%-150%, 80%-150%, 90%-150%, 100%-150%, 30%-100%, 40%-100%, 50%-100%, 60%-100%, 70%-100%, 80%-100%, or 90%-100% increase in efficiency relative to a reference system (e.g., comprising a reference Cas endonuclease) (e.g., the reference Cas endonuclease set forth in SEQ ID NO: 321)).

In some embodiments, the target nucleic acid molecule is a DNA molecule. In some embodiments, the target nucleic acid molecule is a double stranded DNA (dsDNA) molecule. In some embodiments, a portion of the nucleotide sequence of the non-modified strand (as defined herein) of the target dsDNA molecule is complementary to at least a portion of the nucleotide sequence of the first gRNA. In some embodiments, the target nucleic acid molecule is within the genome of cell (e.g., a eukaryotic cell) (e.g., within a subject (e.g., a human subject), plant).

In some embodiments, (b) comprises the first gRNA (e.g., a crRNA and a tracrRNA; or a template RNA (e.g., as described herein)). In some embodiments, (b) comprises the nucleic acid (e.g., DNA) molecule encoding the first gRNA.

In some embodiments, at least a portion of the nucleotide sequence of the first gRNA is complementary to a portion of the nucleotide sequence of the target nucleic acid molecule (e.g., gene). In some embodiments, at least a portion of the nucleotide sequence of the first gRNA is complementary to a portion of the nucleotide sequence of the non-modified strand (as defined herein) of a dsDNA target nucleic acid molecule (e.g., gene). In some embodiments, at least a portion of the nucleotide sequence of the first gRNA binds to a portion of the nucleotide sequence of the non-modified strand (as defined herein) of a dsDNA target nucleic acid molecule (e.g., gene).

In some embodiments, the first gRNA comprises a sgRNA (e.g., a single sgRNA, a plurality of different sgRNAs). In some embodiments, the first gRNA comprises a crRNA (e.g., a single crRNA, a plurality of different crRNAs) and a tracrRNA (e.g., a single tracrRNA, a plurality of different tracrRNAs), wherein the crRNA and the tracrRNA are on separate RNA nucleic acid molecules (or encoded by separate nucleic acid (e.g., DNA) molecules).

In some embodiments, the first gRNA comprises a template RNA (e.g., a single template RNA, a plurality of different template RNAs) that comprises (e.g., from 5′ to 3′) a crRNA, a tracrRNA, a heterologous object sequence, and a 3′ target homology domain. In some embodiments, the template RNA further comprises a sequence that binds a polymerase (e.g., a reverse transcriptase). In some embodiments, the template RNA comprises (e.g., from 5′ to 3′) a crRNA, a tracrRNA, a sequence that binds a polymerase (e.g., a reverse transcriptase), a heterologous object sequence, and a 3′ target homology domain.

In some embodiments, the first gRNA comprises one or more nucleotide comprising one or more chemical modification (e.g., a base, ribose, and/or internucleotide linkage chemical modifications) (i.e., a modified nucleotide). In some embodiments, the modified nucleotide comprises a 2′-O-methyl (2′-OMe); 2′O-methoxyethyl (2′-O-MOE); 2′deoxy-2′-fluoro (2′-F); 2′-arabino-fluoro (2′-Ara-F); 2′-O-benzyl; 2′-O-methyl-4-pyridine (2-O-methyl-4-pyridine (2′-O—CH2Py(4)); 2′F-4′-Cα-OMe; or 2′,4′-di-Cα-OMe, 2′-O-methyl-3′-thioPACE, and/or S-constrained ethyl (cEt). In some embodiments, the modified nucleotide comprises a chemically modified internucleotide (or internucleoside) linkage. In some embodiments, the modified internucleotide (or internucleoside) linkage comprises a phosphorothioate (e.g., a chiral phosphorothioate), a phosphorodithioate, a phosphotriester, an aminoalkylphosphotriester, an alkyl (e.g., methyl) phosphonate (e.g., a 3′-alkylene phosphonate, a chiral phosphonate), a phosphinate, a phosphoroamidate (e.g., a 3′-amino phosphoroamidate, an aminoalkylphosphoramidate), a thionophosphoramidate, a thionoalkylphosphonate, a thionoalkylphosphotriester, or a boranophosphate.

In some embodiments, the first gRNA (e.g., the template RNA, sgRNA) comprises a nucleic acid molecule comprising a toe-loop, hairpin, stem-loop, pseudoknot (e.g., a Mpknot1 moiety), aptamer, G-quadraplex, tRNA, riboswitch, or ribozyme. In some embodiments, the first gRNA (e.g., the template RNA, sgRNA) wherein the nucleic acid molecule is a pseudoknot (e.g., a Mpknot1 moiety).

In some embodiments, the system further comprises a second gRNA (or a nucleic acid (e.g., DNA) molecule encoding the gRNA) that directs the endonuclease of the system to form a single strand break in the non-edited strand of a target dsDNA molecule. In some embodiments, at least a portion of the nucleotide sequence of the second gRNA is complementary to a portion of the nucleotide sequence of the edited strand (as defined herein) of a dsDNA target nucleic acid molecule. In some embodiments, at least a portion of the nucleotide sequence of the second gRNA binds to a portion of the nucleotide sequence of the edited strand (as defined herein) of a dsDNA target nucleic acid molecule. In some embodiments, the second gRNA is present on the same nucleic acid molecule as the first gRNA (or the nucleic acid (e.g., DNA) molecule encoding the second gRNA is present on the same nucleic acid (e.g., DNA) molecule encoding the first gRNA). In some embodiments, the second gRNA is present on a different nucleic acid molecule as the first gRNA (or the nucleic acid (e.g., DNA) molecule encoding the second gRNA is present on a different nucleic acid (e.g., DNA) molecule encoding the first gRNA).

In some embodiments, the system further comprises a donor template nucleic acid (e.g., DNA) molecule (e.g., as defined herein).

In one aspect, provided herein are systems for modifying a dsDNA molecule, comprising: (a) a fusion protein described herein or a nucleic acid molecule (e.g., a DNA, RNA molecule) encoding the fusion protein; and (b) a template RNA (e.g., a single template RNA, a plurality of different template RNAs) that comprises (e.g., from 5′ to 3′) a crRNA, a tracrRNA, a heterologous object sequence, and a 3′ target homology domain; or a nucleic acid molecule (e.g., a DNA molecule) encoding the template RNA.

In one aspect, provided herein are nucleic acid molecules encoding a system described herein. In some embodiments, the nucleic acid molecule is a DNA or RNA (e.g., mRNA) molecule. In some embodiments, the nucleic acid molecule is codon optimized. In some embodiments, the nucleic acid molecule further comprises one or more transcription or translation regulatory elements (e.g., promoter, enhancer (e.g., cell or tissue specific transcription regulatory elements).

In one aspect, provided herein are vectors comprising a nucleic acid molecule described herein. In some embodiments, the vector is a viral vector or a non-viral vector (e.g., plasmid, minicircle). In some embodiments, the vector is a viral vector (e.g., an adeno associated viral (AAV) vector, a lentiviral vector, an adenoviral vector).

In one aspect, provided herein are carriers comprising a system described herein, a nucleic acid molecule described herein, and/or a vector described herein. In some embodiments, the carrier is a nanoparticle, polymer, virus (e.g., a recombinant virus), virus like particle, virosome, fusosome, vesicle, or lipid-based carrier. In some embodiments, the carrier is a recombinant virus (e.g., an adeno associated virus (AAV), a lentivirus, an adenovirus). In some embodiments, the carrier is a nanoparticle. In some embodiments, the carrier is a lipid-based carrier. In some embodiments, the lipid-based carrier is a lipid nanoparticle (LNP), liposome, lipoplex, nanoliposome, an exosome, or a micelle. In some embodiments, the carrier further comprises one or more gRNA (e.g., a crRNA, a tracrRNA, a sgRNA, a template RNA (e.g., as described herein)).

In one aspect, provided herein are reaction mixtures comprising (a) a cell (e.g., comprising a target nucleic acid molecule) or a target nucleic acid molecule; and (b) a system described herein, a nucleic acid molecule described herein, a vector described herein, and/or a carrier described herein.

In one aspect, provided herein are cells comprising a system described herein, a nucleic acid molecule described herein, a vector described herein, a carrier described herein, and/or a reaction mixture described herein.

In one aspect, provided herein are pharmaceutical compositions comprising a system described herein, a nucleic acid molecule described herein, a vector described herein, a carrier described herein, a reaction mixture, and/or a cell described herein; and a pharmaceutically acceptable excipient.

In one aspect, provided herein are kits comprising a system described herein, a nucleic acid molecule described herein, a vector described herein, a carrier described herein, a reaction mixture, a cell described herein, and/or a pharmaceutical composition described herein; and optionally instructions for using any one or more of the foregoing.

In one aspect, provided herein are methods of delivering a Cas endonuclease, fusion protein, conjugate, system, nucleic acid molecule, vector, carrier, reaction mixture, cell, or pharmaceutical composition, to a cell, the method comprising, introducing into a cell a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein, to thereby deliver the Cas endonuclease, fusion protein, conjugate, system, nucleic acid molecule, vector, carrier, reaction mixture, cell, or pharmaceutical composition to the cell.

In some embodiments, the cell is in vitro, ex vivo, or in vivo. In some embodiments, the cell is euploid, is not immortalized, is part of a tissue, is part of an organism, is a primary cell, is non-dividing, is haploid (e.g., a germline cell), is a non-cancerous polyploid cell, or is from a subject having a genetic disease. In some embodiments, the cell is in a subject (e.g., a human subject). In some embodiments, the cell is in a human subject.

In one aspect, provided herein are methods of delivering a Cas endonuclease, fusion protein, conjugate, system, nucleic acid molecule, vector, carrier, reaction mixture, cell, or pharmaceutical composition, to a cell, the method comprising a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein, to thereby deliver the Cas endonuclease, fusion protein, conjugate, system, nucleic acid molecule, vector, carrier, reaction mixture, cell, or pharmaceutical composition to the subject (e.g., human subject).

In one aspect, provided herein are methods of cleaving a target site in a target nucleic acid (e.g., DNA) molecule (e.g., a double stranded target nucleic acid sequence (e.g., dsDNA, (e.g., genomic dsDNA))), the method comprising contacting the cell with a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein, to thereby cleave the target site in the target nucleic acid (e.g., DNA) molecule.

In one aspect, provided herein are methods of editing a target site in a target nucleic acid (e.g., DNA) molecule (e.g., a double stranded target nucleic acid sequence (e.g., dsDNA, (e.g., genomic dsDNA))), the method comprising contacting the cell with a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein, to thereby edit the target site in the target nucleic acid (e.g., DNA) molecule.

In one aspect, provided herein are methods of editing a target site in genomic dsDNA in a cell, the method comprising, contacting a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein, to thereby edit the target site in the genomic DNA of the cell.

In some embodiments, the cell is in vitro, ex vivo, or in vivo. In some embodiments, the cell is euploid, is not immortalized, is part of a tissue, is part of an organism, is a primary cell, is non-dividing, is haploid (e.g., a germline cell), is a non-cancerous polyploid cell, or is from a subject having a genetic disease. In some embodiments, the cell is in a subject (e.g., a human subject). In some embodiments, the cell is in a human subject.

In one aspect, provided herein are methods of editing a target site in a dsDNA molecule (e.g., genomic dsDNA (e.g., in a cell)), the method comprising: contacting a dsDNA molecule with (a) a fusion protein described herein (or a nucleic acid molecule (e.g., a DNA, RNA, nucleic acid molecule) encoding the fusion protein), and (b) a template RNA (e.g., a single template RNA, a plurality of different template RNAs) that comprises (e.g., from 5′ to 3′) a crRNA, a tracrRNA, a heterologous object sequence, and a 3′ target homology domain, to thereby modify the target site in the dsDNA molecule (or a nucleic acid molecule (e.g., a DNA nucleic acid molecule) encoding the template RNA), to thereby edit the target site in the dsDNA molecule (e.g., genomic dsDNA (e.g., in a cell)).

In some embodiments, the nucleic acid molecule is in a cell (e.g., a eukaryotic cell). In some embodiments, the cell is in vitro, ex vivo, or in vivo. In some embodiments, the cell is in a subject (e.g., a human subject). In some embodiments, the cell is in a human subject. In some embodiments, the edit comprises an addition, a deletion, or a substitution of one or more nucleotides into/from the target site of the genomic dsDNA in the cell. In some embodiments, the edit comprises an addition, a deletion, or a substitution of one or more nucleotides into/from the target site of the target nucleic acid molecule. In some embodiments, the addition comprises the addition of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides at the target site. In some embodiments, the deletion comprises the deletion of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides at the target site.

In one aspect, provided herein are methods of treating ameliorating, or preventing a disease in a subject (e.g., a human subject) in need thereof, the method comprising administering to a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein, thereby treat, ameliorate, or prevent the disease in the subject.

In some embodiments, the disease is associated with a genetic defect. In some embodiments, the gRNA of the system is capable of targeting the endonuclease to the site of the genetic defect. In some embodiments, the genetic defect comprises a duplication of a gene, deletion of a gene, or a mutation of a gene. In some embodiments, the administration results in the correction of the genetic defect. In some embodiments, the subject is a human subject.

In one aspect, provided herein are Cas endonucleases, conjugates, fusion proteins, systems, nucleic acid molecules, vectors, carriers, reaction mixtures, cells, or pharmaceutical compositions for use in cleaving a target site in a target nucleic acid (e.g., DNA) molecule (e.g., a double stranded target nucleic acid sequence (e.g., dsDNA, (e.g., genomic dsDNA))) in a subject in need thereof.

In one aspect, provided herein is the use of a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein for the manufacture of a medicament for the cleaving a target site in a target nucleic acid (e.g., DNA) molecule (e.g., a double stranded target nucleic acid sequence (e.g., dsDNA, (e.g., genomic dsDNA))) in a subject in need thereof.

In one aspect, provided herein is a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein for use in editing a target site in a target nucleic acid (e.g., DNA) molecule (e.g., a double stranded target nucleic acid sequence (e.g., dsDNA, (e.g., genomic dsDNA))) in a subject in need thereof.

In one aspect, provided herein is the use of a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein for the manufacture of a medicament for the editing a target site in a target nucleic acid (e.g., DNA) molecule (e.g., a double stranded target nucleic acid sequence (e.g., dsDNA, (e.g., genomic dsDNA))) in a subject in need thereof.

In one aspect, provided herein is a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein for use as a medicament.

In one aspect, provided herein is a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein for use in the treatment of a disease in a subject in need thereof (e.g., a disease is associated with a genetic defect).

In one aspect, provided herein is the use of a Cas endonuclease described herein, a conjugate described herein, a fusion protein described herein, a system described herein, a nucleic acid molecule described herein, a vector described herein, a carrier described herein, a reaction mixture described herein, a cell described herein, or a pharmaceutical composition described herein for the manufacture of a medicament for the treatment of a disease in a subject in need thereof (e.g., a disease is associated with a genetic defect).

### 4. DETAILED DESCRIPTION

Typical CRISPR-Cas editing (e.g., gene editing) systems require a Cas endonuclease to mediate cleavage of the target nucleic acid molecule. Cas endonucleases vary in their ability to mediate target cleavage (e.g., in a cell) depending on e.g., the efficiency of target cleavage, their capability to mediate double and/or single strand breaks, protospacer adjacent motif (PAM) sequence requirements, the specificity of the PAM, etc. As such, a diverse set of Cas endonucleases is useful to provide the ability to select a suitable Cas endonuclease for each specific target nucleic acid molecule; particularly given the incredibly diverse range of potential target nucleic acid molecules (e.g., diverse range of genes).

The inventors have, inter alia, discovered novel Cas endonucleases. As such, the Cas endonucleases described herein can be used to modify, e.g., cleave, DNA, for example, can be used in nucleic acid editing systems (e.g., CRISPR-Cas systems). Accordingly, the current disclosure provides, inter alia, Cas endonucleases capable of cleaving target nucleic acid molecules (e.g., DNA, genes, genomic DNA) (e.g., in a cell, in a cell in a subject); as well as systems and methods of utilizing the same (e.g., methods of cleaving a nucleic acid molecule, methods of editing a nucleic acid molecule (e.g., genomic DNA), and methods of treating diseases (e.g., genetic diseases)).

### Table of Contents

4.1 Definitions
4.2 Cas Endonucleases
4.2.1 Activity of Cas Endonucleases
4.2.1.1 Endonuclease Activity
4.2.1.2 gRNA Binding Activity
4.2.1.3 Target Nucleic Acid Molecule Binding Activity
4.2.1.4 Target Nucleic Acid Editing Activity
4.2.1.5 Alteration of Activity
4.3 Cas Endonuclease Fusion Proteins & Conjugates
4.3.1 Heterologous Proteins
4.3.1.1 Polymerases (e.g., Reverse Transcriptases (RTs))
4.3.1.2 Nucleobase Editors
4.3.2 Linkers
4.3.3 Orientation
4.4 Methods of Making Proteins
4.5 Systems
4.5.1 Target Nucleic Acid Molecules
4.5.2 gRNAs
4.5.2.1 Multiple gRNAs
4.5.2.2 Modified gRNAs
4.5.2.2(i) Nature of the Modifications
4.5.2.2(i)(a) Sugar Modifications
4.5.2.2(i)(b) Nucleobase Modifications
4.5.2.2(i)(c) Internucleoside Linkage Modifications
4.5.2.2(i)(d) Exemplary Combinations of Modifications
4.5.2.2(ii) Location of Modifications
4.5.2.3 Methods of Making gRNAs
4.5.3 Nucleic Acid Editing Activity of Systems
4.5.4 Methods of Assessing Nucleic Acid Editing Activity of Systems
4.5.5 Exemplary Systems
4.5.5.1 HDR Based Editing Systems
4.5.5.2 RT Based Editing Systems
4.5.5.3 Nucleobase Editor Editing Systems
4.6 Nucleic Acid Molecules
4.7 Vectors
4.8 Carriers
4.8.1 Lipid Based Carriers
4.8.1.1 Cationic Lipids (Positively Charged) and Ionizable Lipids
4.8.1.2 Non-Cationic Lipids (e.g., Phospholipids)
4.8.1.3 Structural Lipids
4.8.1.4 Polymers and Polyethylene Glycol (PEG)—Lipids
4.8.1.5 Percentages of Lipid Nanoformulation Components
4.9 Cells
4.10 Reaction Mixtures
4.11 Pharmaceutical Compositions
4.12 Kits
4.13 Methods of Use
4.13.1 Methods of Delivery
4.13.2 Methods of Cleaving a Target Nucleic Acid Molecule
4.13.3 Methods of Editing a Target Nucleic Acid Molecule
4.13.3.1 Methods of Editing a Target Nucleic Acid Molecule Utilizing an RT-Based System
4.13.3.2 Methods of Editing a Target Nucleic Acid Molecule Utilizing an HDR-Based System
4.13.3.3 Methods of Editing a Target Nucleic Acid Molecule Utilizing a Nucleobase Editor-Based System
4.13.4 Methods of Treating, Ameliorating, or Preventing a Disease

### 4.1 Definitions

The section headings used herein are for organizational purposes and do not limit the subject matter described.

Unless defined otherwise, all technical and scientific terms used herein have the same meaning as is understood by one of skill in the art to which the claimed subject matter belongs. It is to be understood that the general and detailed descriptions are exemplary and explanatory and are not restrictive of claimed subject matter.

In this application, the use of the singular includes the plural unless stated otherwise. For example, as used in the disclosure, the singular forms “a,” “an,” and “the” include plural referents unless the context dictates otherwise. Furthermore, use of the term “including” as well as other forms, such as “include,” “includes,” and “included,” is not limiting.

It is understood that aspects and embodiments described herein with “comprising” language, also otherwise include analogous aspects and embodiments described in terms of “consisting of” and “consisting essentially of”.

The term “and/or” is to be taken as specific disclosure of each of the two specified features or components with or without the other. Thus, “and/or” as used in a phrase such as “A and/or B” herein is intended to include “A and B,” “A or B,” “A” (alone), and “B” (alone). Likewise, “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, concentration ranges, percentage ranges, ratio ranges or integer ranges are understood to include the value of any integer within the recited range and, when appropriate, fractions thereof (such as one tenth and one hundredth of an integer), unless otherwise indicated.

The terms “about” refers to a value or composition that is within an acceptable error range for the particular value or composition as understood and/or determined by one of ordinary skill in the art, which will depend in part on how the value or composition is measured or determined, i.e., limitations of the measurement system. When particular values or compositions are provided in the disclosure, unless otherwise stated, the meaning of “about” is understood to be within an acceptable error range for that particular value or composition.

Where proteins are described herein, it is understood that polynucleotides (e.g., RNA or DNA nucleic acid molecules) encoding the proteins are also provided herein.

Where proteins, nucleic acid molecules, vectors, carriers, etc. are described herein, it is understood that isolated forms of the proteins, nucleic acid molecules, vectors, carriers, etc. are also provided herein.

Where proteins, nucleic acid molecules, etc. are described herein, it is understood that recombinant forms of the proteins, nucleic acid molecules, etc. are also provided herein.

Where proteins or sets of proteins are described herein, it is understood that both proteins comprising the primary structure are provided herein as well as proteins folded into their three-dimensional structure (i.e., tertiary or quaternary structure) are provided herein.

As used herein, the term “administering” refers to the physical introduction of an agent, e.g., a therapeutic agent (or a precursor of the therapeutic agent that is metabolized or altered within the body of the subject to produce the therapeutic agent in vivo) (e.g., systems comprising endonucleases for introducing variations into a target nucleic acid) to a subject, using any of the various methods and delivery systems known to those skilled in the art. Administering can also be performed, for example, once, a plurality of times, and/or over one or more extended periods. Therapeutic agents include agents whose effect is intended to be preventative (i.e., prophylactic), such as agents for modifying target nucleic acids (e.g., systems comprising endonucleases for introducing a variation into a target nucleic acid).

As used herein, the term “bicyclic sugar” refers to a modified sugar (e.g., ribose) moiety comprising two rings, wherein the second ring is formed via a bridge connecting two of the atoms in the first ring thereby forming a bicyclic structure. In some embodiments, the first ring of the bicyclic sugar moiety is a furanosyl moiety. In some embodiments, the furanosyl sugar moiety is a ribosyl moiety.

As used herein, the term “bicyclic nucleoside” (“BNA”) is a nucleoside comprising a bicyclic sugar.

As used herein, the term “crRNA” refers to an RNA molecule (e.g., part of a gRNA (e.g., a sgRNA)) that is capable of binding to the protospacer in a target nucleic acid (e.g., DNA) molecule.

As used herein, the term “disease” refers to an abnormal condition that impairs physiological function. The term encompasses any disorder, illness, abnormality, pathology, sickness, condition, or syndrome in which physiological function is impaired, irrespective of the nature of the etiology. The term disease includes infection (e.g., a viral, bacterial, fungal, protozoal infection).

As used herein, the term “donor template nucleic acid molecule” refers to a nucleic acid molecule that contains a donor region comprising a nucleic acid sequence of interest (e.g., contains a nucleotide variation of interest (e.g., a substitution, addition, deletion, inversions, etc.)) and two homology arms each comprising a nucleotide sequence of sufficient homology to the nucleotide sequence of the region flanking the target cleavage site of an endonuclease described herein (also referred to herein as homology arms). Each of the homology arms flank the donor region, such that the donor region is between the two homology arms. In some embodiments, the donor template nucleic acid molecule is a donor DNA template nucleic acid molecule. In some embodiments, the donor template nucleic acid molecule is an RNA template molecule. In some embodiments, the donor template nucleic acid molecule is double stranded. In some embodiments, the donor template nucleic acid molecule is single stranded. In some embodiments, the donor template nucleic acid molecule can be utilized in a system described herein (e.g., an HDR based system described herein), wherein the molecular machinery of the cell can utilize the exogenous donor template nucleic acid in repairing and/or resolving a cleavage site in a target nucleic acid molecule mediated by an endonuclease (or functional fragment, functional variant, or domain thereof) (e.g., of the system).

The terms “DNA” and “polydeoxyribonucleotide” are used interchangeably and refer to macromolecules including multiple deoxyribonucleotides that are polymerized via phosphodiester bonds. Deoxyribonucleotides are nucleotides in which the sugar is deoxyribose.

As used herein, the term “domain” refers to a structure of a biomolecule (e.g., a protein, nucleic acid (e.g., DNA, RNA)) molecule) that contributes to a specified function of the biomolecule (e.g., a protein, nucleic acid (e.g., DNA, RNA)). A domain may comprise a contiguous region (e.g., a contiguous sequence) or distinct non-contiguous regions (e.g., non-contiguous sequences) of a biomolecule. Examples of protein domains include, but are not limited to, an endonuclease domain, a DNA binding domain, a reverse transcriptase domain; an example of a domain of a nucleic acid is a regulatory domain, such as a transcription factor binding domain. In some embodiments, a domain (e.g., a Cas domain) can comprise two or more smaller domains (e.g., a DNA binding domain and an endonuclease domain).

As used herein, the term “editing” with reference to a nucleic acid molecule (e.g., a target nucleic acid (e.g., DNA) molecule (e.g., a double stranded target nucleic acid sequence (e.g., dsDNA, (e.g., genomic dsDNA))) refers to the introduction of a variation (as defined herein) (also referred to as an edit herein) in the nucleic acid molecule. In some embodiments, the variation or edit comprises a substitution, addition, deletion, or inversion.

As used herein, the term “edited strand” with reference to a double stranded nucleic acid molecule (e.g., a dsDNA molecule) refers to the strand of the double stranded nucleic acid molecule that is edited by e.g., an endonuclease, system, etc. described herein. Likewise, as used herein, the term “non-edited strand” with reference to a double stranded nucleic acid molecule (e.g., a dsDNA molecule) refers to the strand of the double stranded nucleic acid molecule that is not edited by e.g., an endonuclease, system, etc. described herein.

As used herein, the term “functional fragment” in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein. For example, a functional fragment of a polymerase, reverse transcriptase or endonuclease can refer to a fragment of said protein that retains activity. In some embodiments, the functional fragment comprises one or more domains (e.g., 1, 2, 3, or more) of the reference protein.

As used herein, the term “functional variant” in reference to a protein refers to a protein that comprises at least one but not more than 20%, not more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively altered, reduced or eliminated (e.g., endonuclease activity). In some embodiments, the reference protein is a wild type protein. In some embodiments, the functional variant comprises one or more domains (e.g., 1, 2, 3, or more) of the reference protein.

As used herein, the term “functional fragment or variant thereof” and the like with reference to an agent (e.g., a protein) should be understood to include functional variants, functional variants, functional fragments, and variants.

As used herein, the term “fuse” and grammatical equivalents thereof refers to the operable connection of at least a first polypeptide to a second polypeptide, wherein the first and second polypeptides are not naturally found operably connected together. For example, the first and second polypeptides are derived from different proteins and/or are from different organisms. The term fuse encompasses both a direct connection of the at least two polypeptides through a peptide bond, and the indirect connection through a linker (e.g., a peptide linker).

As used herein, the term “fusion protein” and grammatical equivalents thereof refer to a protein that comprises at least one polypeptide operably connected to another polypeptide, wherein the first and second polypeptides are not naturally found operably connected together. For example, the first and second polypeptides of the fusion protein are each derived from different proteins and/or are from heterologous organisms. In some embodiments, the first and second polypeptides are different. For the sake of clarity, it will be understood that neither the first nor second polypeptide is required to be a full-length protein (e.g., a full-length naturally occurring protein). For example, the first and/or second polypeptide can comprise or consist of fragments (e.g., functional fragments or domains of full-length proteins (e.g., engineered, naturally occurring). The at least two polypeptides of the fusion protein can be directly operably connected through a peptide bond; or can be indirectly operably connected through a linker (e.g., a peptide linker). Thus, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “guide RNA” or “gRNA” refers to an RNA molecule that can associate with an endonuclease (e.g., an endonuclease described herein) to direct the endonuclease (e.g., an endonuclease described herein) to a target nucleic acid molecule (e.g., within a gene (e.g., within a cell)). A gRNA requires a crRNA and a tracrRNA. As described throughout, the crRNA and tracrRNA may be part of the same larger RNA molecule (e.g., a sgRNA) or separate RNA molecules.

As used herein, the term “heterologous,” when used to describe a first element in reference to a second element means that the first element and second element do not exist in nature disposed as described. For example, a protein comprising a “heterologous moiety” means a protein that is joined to a moiety (e.g., small molecule, protein, polynucleotide, carbohydrate, lipid, synthetic polymer (e.g., polymers of PEG), etc.) that is not joined to the protein in nature.

As used herein, the term “heterologous object sequence” refers to an RNA molecule that encodes a desired edit (e.g., substitution, addition, deletion of one or more nucleotides) of a target nucleic acid (e.g., DNA) sequence (e.g., a gene) that can be utilized as a template strand by a polymerase (e.g., a reverse transcriptase) (e.g., described herein) to polymerize the desired nucleic acid sequence (e.g., DNA sequence (e.g., gene sequence)) (i.e., to polymerize sequence complementary to the edit template). In some embodiments, the edit template is part of a template gRNA (e.g., described herein).

It is clear from the disclosure, but for the sake of clarity, it is to be understood that the use of the term “heterologous protein” (e.g., any heterologous protein described herein) includes the full-length protein, as well as less than the full-length protein, including, e.g., functional fragments, functional variants, and domains of the full-length protein.

As used herein, the term “isolated” with reference to a biomolecule (e.g., a protein or polynucleotide) refers to a biomolecule (e.g., a protein or polynucleotide) that is substantially free of other cellular components with which it is associated in the natural state.

As used herein, the term “translatable RNA” refers to any RNA that encodes at least one polypeptide and can be translated to produce the encoded protein in vitro, in vivo, in situ or ex vivo. A translatable RNA may be an mRNA or a circular RNA encoding a polypeptide.

As used herein, the terms “agent” and “moiety” are used interchangeably herein and refer to any macro or micro molecule that can be operably connected to another macro or micro molecule (e.g., a protein (e.g., an endonuclease (or a functional fragment, functional variant, or domain thereof)) or a nucleic acid molecule encoding the protein (e.g., endonuclease)). Exemplary moieties include, but are not limited small molecules, proteins, polynucleotides (e.g., DNA, RNA), carbohydrates, lipids, synthetic polymers (e.g., polymers of PEG).

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, including a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan appreciates that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “nucleobase editor” refers to an agent (e.g., a biomolecule (e.g., a protein (or a functional fragment, functional variant, or domain thereof))) that can mediate nucleobase editing activity.

As used herein, the term “nucleobase editing activity” refers to the ability of an agent (e.g., a biomolecule (e.g., a protein (or a functional fragment, functional variant, or domain thereof))) to chemically alter a nucleobase within a polynucleotide. In some embodiments, the nucleobase editing activity is cytidine deaminase activity, e.g., converting a target C·G to T·A. In some embodiments, the nucleobase editing activity is adenosine deaminase activity, e.g., converting A·T to G·C. In some embodiment, the nucleobase editing activity is cytidine deaminase activity and adenosine deaminase activity, e.g., converting A·T to G·C.

As used herein, the term “operably connected” refers to the linkage of two moieties in a functional relationship. For example, a polypeptide is operably connected to another polypeptide when they are linked (either directly or indirectly via a peptide linker) such that both polypeptides are functional (e.g., an in-frame fusion protein comprising an endonuclease described herein). Or for example, a transcription regulatory polynucleotide e.g., a promoter, enhancer, or other expression control element operably linked to a polynucleotide that encodes a protein to affect the transcription of the polynucleotide that encodes the protein. The term “operably connected” also refers to the conjugation of a moiety to e.g., a polynucleotide or polypeptide (e.g., the conjugation of a PEG polymer to a protein).

As used herein, the term “PAM” or “protospacer adjacent motif” refers to a short nucleic acid molecule (usually about 2-6 base pairs in length) that follows the nucleic acid region targeted for cleavage by an endonuclease (e.g., described herein (e.g., of a system described herein)). In some embodiments, the PAM is required for an endonuclease (e.g., described herein (e.g., of a system described herein)) to cleave the target nucleic acid molecule and is generally located near (e.g., 3-4 nucleotides) downstream of the cleavage site.

Determination of “percent identity” between two sequences (e.g., protein (amino acid sequences) or polynucleotide (nucleic acid sequences)), as used herein, can be accomplished using a mathematical algorithm. For example, a specific, non-limiting example of an algorithm utilized for the comparison of two sequences is described in Karlin S & Altschul S F (1990) PNAS 87: 2264-2268, modified as in Karlin S & Altschul S F (1993) PNAS 90: 5873-5877, each of which is herein incorporated by reference in its entirety. Such algorithm(s) is incorporated into the NBLAST and XBLAST programs of Altschul S F et al., (1990) J Mol Biol 215: 403, which is incorporated herein by reference in its entirety. BLAST nucleotide searches are performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. For gapped alignment comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform searches which detect distant relationships between molecules (Id.). When utilizing BLAST, Gapped BLAST, and PSI Blast programs, default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is described in Myers and Miller, 1988, CABIOS 4:11-17, which is herein incorporated by reference in its entirety. Such an algorithm is incorporated in the ALIGN program (version 2.0) and is a part of the GCG sequence alignment software package. When comparing amino acid sequences with the ALIGN program, a PAM120 weight residue table, a gap length penalty of 12, and a gap penalty of 4 can be used. Percent identity between two sequences can be determined using techniques similar to those described above, with or without allowing gaps. In calculating percent identity, typically only exact matches are counted.

As used herein, the term “plurality” means 2 or more (e.g., 3 or more, 4 or more, 5 or more, 6 or more, 7 or more, 9 or more, or 10 or more).

As used herein, the term “pharmaceutical composition” refers to a composition that is suitable for administration to an animal, e.g., a human subject, and comprises an agent (e.g., therapeutic agent) and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, “protein” and “polypeptide” refer to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, a protein is folded into its three-dimensional structure. Where proteins are contemplated herein, it should be understood that proteins comprising the primary structure are provided herein as well as proteins folded into their three-dimensional structure (i.e., tertiary or quaternary structure) are provided herein.

As used herein, the term “prophylactic treatment” and the like refers to a treatment administered to a subject for the purpose of decreasing the risk of developing pathology in a subject who does not exhibit signs of a disease or exhibits only early signs of a disease.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “sgRNA” refers to a gRNA molecule that comprises both a crRNA and a tracrRNA. The components of the sgRNA may be arranged in any suitable order and any component may be operably connected to the adjacent component(s) directly or indirectly (e.g., via a nucleotide linker).

As used herein, the term “signal peptide” or “signal sequence” refers to a sequence that can direct the transport or localization of a protein, such as an endonuclease, to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide. Exemplary signal sequences include for example, nuclear localization signal and nuclear export signal.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “template RNA” refers to gRNA molecule that comprises a crRNA, a tracrRNA, a heterologous object sequence, and a 3′ target homology domain. In some embodiments, the template RNA further comprises an RNA sequence that binds a polymerase (e.g., a reverse transcriptase, e.g., of a fusion protein described herein). The components of the template RNA may be arranged in any suitable order and any component may be operably connected to the adjacent component(s) directly or indirectly (e.g., via a nucleotide linker). In some embodiments, the template RNA comprises from 5′ to 3′ a crRNA, a tracrRNA, a heterologous object sequence, and a 3′ target homology domain. In some embodiments, the template RNA comprises from 5′ to 3′ a crRNA, a tracrRNA, a sequence that binds a polymerase (e.g., a reverse transcriptase, e.g., of a fusion protein described herein), a heterologous object sequence, and a 3′ target homology domain. In some embodiments, the template RNA is part of a system (e.g., a reverse transcriptase-based system) described herein.

As used herein, the term “therapeutically effective amount” of an agent (e.g., therapeutic agent) refers to any amount of the agent (e.g., therapeutic agent) that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the term “tracrRNA” refers to an RNA molecule (e.g., part of a gRNA (e.g., a sgRNA)) that mediates binding of a gRNA to an endonuclease (e.g., an endonuclease described herein).

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

As used herein, “variant” or “variation” with reference to a nucleic acid molecule (e.g., a nucleic acid molecule encoding an endonuclease as described herein), refers to a nucleic acid molecule that comprises at least one substitution, inversion, addition, or deletion of nucleotide compared to a reference nucleic acid molecule. As used herein, the term “variant” or “variation” with reference to a protein refers to a peptide or protein (e.g., endonucleases described herein) that comprises at least one substitution, inversion, addition, or deletion of an amino acid residue compared to a reference protein.

As used herein, the term “3′ target homology domain” refers to an RNA molecule that is capable of hybridizing to the 3′ end of a single stranded nucleic acid flap (the 3′target sequence) created after induction of a single strand break (i.e., a nick) in a target double stranded nucleic acid (e.g., DNA) molecule (e.g., by an endonuclease described herein (or a fusion protein comprising the same)). The hybridization of the 3′ target homology domain to the 3′ target sequence creates a duplex that can be utilized as a substrate by a polymerase (e.g., a reverse transcriptase) (e.g., described herein) for polymerization of a nucleic acid (e.g., DNA) molecule (e.g., utilizing the heterologous object sequence). In some embodiments, the 3′ target homology domain is part of a template RNA (e.g., described herein).

### 4.2 Cas Endonucleases

Provided herein are, inter alia, Cas endonucleases (and functional fragments, functional variants, and domains thereof), useful in, inter alia, modifying (e.g., editing) a nucleic acid molecule (e.g., DNA, gene, genome (e.g., within a cell, e.g., within a cell in a subject (e.g., a mammalian subject, e.g., a human subject))) (e.g., in vivo, ex vivo, or in vitro). In some embodiments, the Cas endonuclease is non-naturally occurring. The amino acid sequence of exemplary Cas endonucleases of the disclosure is set forth in Table 1 and in SEQ ID NOS: 1-320.

TABLE 1
The Amino Acid Sequence of Cas Endonucleases.

| | | SEQ |
|---|---|---|
| Description | Amino Acid Sequence | ID NO |
| CasEnd-41 | MKKKYSIGLDIGTNSVGWAVITDEYKVPSKKFKVLGNTDRQSIKKNLIGALL | 1 |
| | FDSGETAEATRLKRTARRRYTRRKNRIRYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINEEGVDA | |
| | KAILSARLSKSRRLENLIAQLPGEKKNGLFGNLIALSLGLTPNFKSNFDLAE | |
| | DAKLQLSKDTYDEDLDNLLAQIGDQYADLFLAAKNLSDAILLSDILRVNSEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPTEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKEFLDNEENEDILEDIVLTLT | |
| | LFEDKEMIEERLKKYANLFDDKVLKQLKRRRYTGWGRLSKKLINGIRDKQSG | |
| | KTILDFLKSDGFANRNFMQLIHDDSLTFKEEIKKAQVKGQGDSLHEQIANLA | |
| | GSPAIKKGILQTVKIVDEIVKVMGRHKPENIVIEMARENQTTQKGQKNSRER | |
| | MKRLEEGIKELGSQILKEHPVENTQLQNEKLYLYYLQNGKDMYTGQELDIDR | |
| | LSDYDVDHIIPQSFIKDDSIDNKVLTSSKEARGKSDNVPSEEVVKKMKSYWR | |
| | QLLNAKLISQRKFDNLTKAERGGLSELDKAGFIKRQLVETRQITKHVAQILD | |
| | SRMNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKSKKLKSVKELVGITIMERSSFEKDPVAFLEAKGYKEIQKDLII | |
| | KLPKYSLFELENGRKRLLASAGELQKGNELALPSKYVTFLYLASHYEKLKGS | |
| | PEDNEQKQLFVEQHRHYLDEIVDQISEFSKRYILADANLDKVLSLYNKHRDK | |
| | PIREQAENIINLETLTNLGAPAAFKFFDTTIDRKRYTSTKEILDATLIHQSI | |
| | TGLYETRIDLSQLGGD | |
| CasEnd-42 | MDKKYSIGLDIGTNSVGWAVITDEYKVPSKKFKVLGNTDRQSIKKNLLGALL | 2 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENSDVQKLFIQLVQTYNQLFEENPINESGVDA | |
| | KAILSAKLSKSRRLENLIAQFPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDDKVLKQLKRRRYTGWGRLSKKLINGIRDKQSG | |
| | KTILDFLKSDGFANRNFMQLIHDDSLTFKEEIKKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTSSEKARGKSDNVPSEEVVKKMKNYWR | |
| | QLLNAKLISQRKFDNLTKAERGGLSELDKAGFIKRQLVETRQITKHVAQILD | |
| | SRMNTKRDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEIKLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEAKGYKEIQKDLII | |
| | KLPKYSLFELENGRKRLLASAGELQKGNELALPSKYVTFLYLASHYEKLKGS | |
| | PEDNEKKQLYVEQHKHYFDEIVDQISEFSKRYILADKNLDKILSLYNNFEDK | |
| | PIREQAENFINLFTLTALGAPAAFKFFDTTIDRKRYTSTKEILNATLIHQSI | |
| | TGLYETRIDLSQLGGD | |
| CasEnd-43 | MDKKYSIGLDIGTNSVGWAVITDEYKVPSKKFKVLGNTDRKSIKKNLIGALL | 3 |
| | FDSGETAEATRLKRTARRRYTRRKNRIRYLQEIFASEMAKVDDSFFHRLEES | |
| | FLVEEDKSNERHPIFGNIVDEVAYHEKYPTIYHLRKKLVDSDEKADLRLIYL | |
| | ALAHMIKFRGHFLIEGDLNPDNSDVDKLFIQLVETYNQLFEEKPINASGVDA | |
| | KAILSAKLSKSRRLENLIAELPGEKKNGLFGNLIALSLGLTPNFKSNEDLSE | |
| | DAKLQLSKDTYDDDLENLLGQIGDQYADLFSAAKNLSDAILLSDILRVKTEI | |
| | TKAPLSASMIKRYDEHHQDLTLLKALVRQQLPEKYKEIFFDQSKNGYAGYID | |
| | GGASQEEFYKFIKKILEKMDGTEELLDKLNREDLLRKQRTFDNGSIPHQIHL | |
| | GELHAILRRQEKYYPFLKENREKIEKILTFRIPYYVGPLARGNSRFAWMTRK | |
| | SDETITPWNFDEVVDKGASAQSFIERMTNFDKNLPDEKVLPKHSLLYEYFTV | |
| | YNELTKVKYVTEGMRKPSFLSGEQKKAIVNLLFKKNRKVTVKQLKEYYFKEI | |
| | EEFDSVEISGVEDRFNASLGTYHDLLKIIKDKSFLDNEENEKILEDIVLTLT | |
| | LFEDREMIKKRLEKYANLFDKKVMKQLKRRRYTGWGRLSRKLINGIRDKQSG | |
| | KTILDFLKDDGFTNRNFMQLIHDDSLTFKDDIEKAQVSDQGESLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENGKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNGETGEIVWDKERDFATVRKVLSMPQV | |
| | NIVKKTEVQTGGFSKESILPKRDSDKLIARKKDWDPKKYGGFDSPTVAYSVL | |
| | VVAKVEKGKAKKLKTVKELLGITIMERSAFEKNPIAFLEAKGYKNVKKDLII | |
| | KLPKYSLFELENGRKRMLASAGELQKGNELALPSKYVNFLYLASHYEKLKGS | |
| | SEDNKQKQLYVEQHKEYLDEIIDQISEFSERVILADANLEKVLEAYDKHRDK | |
| | SIEEQAENIIHLFTLTNLGAPAAFKYFGTTIDRKRYTSTKEVLNATLIHQSI | |
| | TGLYETRIDLSQLGGD | |
| CasEnd-44 | MKKKYSIGLDIGTNSVGWAVITDEYKVPSKKFKVLGNTDRQSIKKNLLGALL | 4 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINEEGVDA | |
| | KAILSAKLSKSRRLENLIAQLPGEKKNGLFGNLIALSLGLTPNFKSNEDLAE | |
| | DAKLQLSKDTYDEDLENLLAQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYANLFDDKVLKQLKRRRYTGWGRLSKKLINGIRDKQSG | |
| | KTILDFLKSDGFANRNFMQLIHDDSLTFKEEIKKAQVKGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGDELDIDR | |
| | LSDYDVDHIIPQSFIKDDSIDNKVLTSSEKARGKSDNVPSEEVVKKMKNYWR | |
| | QLLNAKLISQRKFDNLTKAERGGLSELDKAGFIKRQLVETRQITKHVAQILD | |
| | SRFNTERDENDKLIRDVKIITLKSKLVSDFRKDFQFYKVREINDYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKDPVAFLEAKGYKEIQKDLII | |
| | KLPKYSLFELENGRKRLLASAGELQKGNELALPSKYVTFLYLASHYEKLKGS | |
| | PEDNEQKQLFVEQHKHYFDEILDQISEFSERYILADKNLEKILSLYNKNRDK | |
| | SISEQAESIINLFTLTALGAPAAFKFFDTTIDRKRYTSTKEILDATLIHQSI | |
| | TGLYETRIDLSQLGGD | |
| CasEnd-45 | MDKKYSIGLDIGTNSVGWAVITDEYKVPSKKFKVLGNTDRHSIKKNLIGALL | 5 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVQKLFIQLVQTYNQLFEENPLDEETVDA | |
| | KAILTAKLSKSRRLENLIAQLPGEKKNGLFGNLIALSLGLTPNFKSNEDLSE | |
| | DAKLQLSKDTYDEDLDNLLGQIGDQYADLFLAAKNLSDAILLSGILTVNDEI | |
| | TKAPLSASMIKRYDEHHQDLTLLKALVRQQLPEKYKEIFFDKSKNGYAGYID | |
| | GGASQEEFYKYIKPILEKMDGAEELLAKINREDFLRKQRTFDNGSIPHQIHL | |
| | GELHAILRRQEKFYPFLKENREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMGKPEFFSGEQKQEIVDLLFKKNRKVTVKQLKEFLFKEI | |
| | ECFDSVEISGVEDRFNASLGTYHDLLKIIKDKEFLDNEENEDILEDIVLTLT | |
| | LFEDREMIEERLKKYANLFDKKVLKKLKRRRYTGWGRLSRKLINGIRDKQSG | |
| | KTILDFLKSDGFANRNFMQLIHDDSLSFKEEIQKAQVSGQGDSLHEHIANLA | |
| | GSPAIKKGILQTVKVVDELVKVMGRHKPENIVIEMARENQTTQKGQKNSRER | |
| | MKRLEEGLKELGSQILKEHPVENTQLQNDKLYLYYLQNGKDMYTGEELDIDR | |
| | LSDYDVDHIIPQSFIKDDSIDNKVLTSSAKARGKSDNVPSEEVVKKMKSYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKIEKGKAKKLKTVKELVGITIMERSSFEKDPVAFLEAKGYKNIQKDLII | |
| | KLPKYSLFELENGRKRLLASAGELQKGNELALPNKYVTLLYHAKHYEKLKGS | |
| | PEDNEKHLEYVEQHRHEFDEILEQISEFSERYILADKNLEKILELYNKNEDY | |
| | SISELAESFINLFTLTALGAPAAFKFFGTTIDRKRYTSTKEILDATLIHQSI | |
| | TGLYETRIDLSQLGGD | |
| CasEnd-46 | MDKKYSIGLDIGTNSVGWAVITDEYKVPSKKFKVLGNTDRHSIKKNLIGALL | 6 |
| | FDSGETAEATRLKRTARRRYTRRKNRICYLQEIFSNEMAKVDDSFFHRLEES | |
| | FLVEEDKRHERHPIFGNIVDEVAYHEKYPTIYHLRKHLADSTEKADLRLIYL | |
| | ALAHIIKFRGHFLIEGDLNSDNSDVDKLFIQLVQTYNQLFEENPINEEGVDA | |
| | KAILTEKLSKSRRLENLIAQFPGEKRNGLFGNLIALSLGLTPNFKSNFGLAE | |
| | DAKLQLSKDTYDEDLENLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKYIKPILEKMDGSEYFLAKINREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPSEKVLPKHSLLYEYFTV | |
| | YNELTKVKYVTEGMGKPEFFSAEQKQEIVDLLFKKNRKVTVKRLKEFLFKEI | |
| | ECFRSVEISGVEDAFNASLGTYHDLLKIIKDKDELDNEENEKILEDIVLTLT | |
| | LFEDREMIEERLEKYANLFDKKVLKKLKRRRYTGWGRLSKKLINGIRDKQSG | |
| | KTILDFLKSDGFANRNFMQLIHDDSLTFKDEIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-47 | MDKKYSIGLDIGTNSVGWAVITDEYKVPSKKFKVLGNTDRHSIKKNLLGALL | 7 |
| | FDSGETAEATRLKRTARRRYTRRKNRIRYLQEIFSEEMAKVDDSFFHRLEES | |
| | FLVEEDKKHERHPIFGNIVDEVAYHENYPTIYHLRKKLADSTEKADLRLIYL | |
| | ALAHIIKFRGHFLIEGDLNPENSDVQKLFIQLVQTYNQLFEENPINESGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILTVNDEI | |
| | TKAPLSASMIKRYDEHHQDLTLLKALVRQQLPEKYKEIFFDKSKNGYAGYID | |
| | GGASQEEFYKFIKPILEKMDGTEELLAKINREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLEKYAHLFDKKVLKQLKRRRYTGWGRLSRKLINGIRDKQSG | |
| | KTILDFLKSDGFANRNFMQLIHDDSLTFKEEIKKAQVIGQGDSLHEQIANLA | |
| | GSPAIKKGILQSIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIREVKIITLKSKLVSDFRKDFQFYKVREINDYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPIDFLEAKGYKNIQKDLII | |
| | KLPKYSLFELENGRKRMLASAGELQKGNELALPQKYVTLLYLASHYEKLKGS | |
| | PEDNSQKLEYVEQHRYYFDEIFEQISEFSERYILADKNLDKVKSLYNNHRDK | |
| | PIREQAENFIHLFTFTSLGAPAAFKFFDTTIDRKRYTSTTEVLDATLIHQSI | |
| | TGLYETRIDLSQLGGD | |
| CasEnd-48 | MDKKYSIGLDIGTNSVGWAVITDEYKVPSKKFKVLGNTDRHSIKKNLIGALL | 00 |
| | FDSGETAEATRLKRTARRRYTRRKNRIRYLQEIFANEMAKVDDSFFHRLEES | |
| | FLVEEDKKHERHPIFGNIVDEVAYHEKYPTIYHLRKKLVDSDEKADLRLIYL | |
| | ALAHMIKFRGHFLIEGDLNSDNSDVQKLFIQLVQTYNQLFEESPIEAEKVDA | |
| | KAILSAKLSKSRRLENLIAQLPGEKKNGLFGNLIALSLGLTPNFKSNFDLAE | |
| | DAKLQLSKDTYDDDLENLLGQIGDQYADLFLAAKNLSDAILLSGILRVNTEI | |
| | TKAPLSASMIKRYDEHHQDLTLLKQLVRQQLPEKYKEIFFDQSKNGYAGYID | |
| | GGASQEEFYKYIKPILEKMDGTEELLAKLEREDLLRKQRTFDNGSIPHQIHL | |
| | GELHAILRRQEKYYPFLKENREKIEKILTFRIPYYVGPLARGNSRFAWMTRK | |
| | SDETITPWNFEEVVDKGASAQKFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIKERLEKYADLFDKKVMKQLKRRRYTGWGRLSKKLINGIRDKQSG | |
| | KTILDFLKADGFANRNFMQLIHDDSLTFKEEIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTEYDENDKLIRDVKVITLKSKLVSDFRKDFGFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNEETGEIVWDKEKDFATVRKVLSMPQV | |
| | NIVKKTEVQTGGFSKESILPKRDSDKLIARKKDWDPKKYGGFDSPTVAYSVL | |
| | VVAKVEKGKTKKLKTVKELLGITIMERSAFEKNPVDFLEAKGYKEVKKDLII | |
| | KLPKYSLFELENGRKRMLASAGELQKGNELALPSKYVNFLYLASHYEKLKGK | |
| | PEDNEQKQLFVEQHKEYLDEIIDQISEFSKRVILADANLEKVKSAYNKHRDK | |
| | SIEEQAENIIHLFTLTALGAPAAFKYFDTTIDRKRYTSTKEVLNATLIHQSI | |
| | TGLYETRIDLSQLGGD | |
| CasEnd-49 | MDKKYSIGLDIGTNSVGWAVITDEYKVPSKKFKVLGNTDRHSIKKNLIGALL | 9 |
| | FDSGETAEATRLKRTARRRYTRRKNRIRYLQEIFSEEMAKVDDSFFHRLEES | |
| | FLVEEDKKNERHPIFGNIVDEVAYHEKYPTIYHLRKKLADSDEKADLRLIYL | |
| | ALAHMIKFRGHFLIEGDLNSDNSDVQKLFIQLVQTYNQLFEENPLNEIGVDA | |
| | KAILTARLSKSRRLENLIAQFPGEKKNGLFGNLIALSLGLTPNFKSNFDLSE | |
| | DAKLQLSKDTYDEDLENLLAQIGDQYADLFLAAKNLSDAILLSDILTVNDEI | |
| | TKAPLSASMVKRYDEHHQDLTLLKALVRQQLPEKYKEIFFDQSKNGYAGYID | |
| | GGASQEEFYKYLKPILEKMDGSEEFLEKINREDFLRKQRTFDNGSIPHQIHL | |
| | GELHAILRRQEKYYPFLKENREKIEKILTFRIPYYVGPLARGNSRFAWMTRK | |
| | SDETITPWNFEEVVDKEASAQSFIERMTNFDKNLPNEKVLPKHSLLYEKFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVLKQLKRRRYTGWGRLSKKLINGIRDKQSG | |
| | KTILDFLKADGFANRNFMQLIHDDSLTFKEEIEKAQVSGQGESLHELIANLA | |
| | GSPAIKKGILQTIKIVDELVKVMGRHAPENIVIEMARENQTTQKGQKNSRER | |
| | MKRLEEGIKELGSKILKEHPVENTQLQNEKLYLYYLQNGKDMYTGEELDIDR | |
| | LSDYDVDHIVPQSFLKDDSIDNKVLTSSAKARGKSDNVPSEEVVKKMKNYWR | |
| | KLLDAKLITQRKFDNLTKAERGGLTELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKNPVDFLEAKGYKEVKKDLII | |
| | KLPKYSLFELENGRKRLLASAGELQKGNELALPQKYVEFLYLASHYEKLKGS | |
| | PEDNEQKQLFVEQHKHYFDEIIEQISEFSKRYILADANLEKILSLYEKNRDK | |
| | PIEEQAESFINLFTLTALGAPAAFKFFDTTIDRKRYTSTKEILDATLIHQSI | |
| | TGLYETRIDLSQLGGD | |
| CasEnd-50 | MDKKYSIGLDIGTNSVGWAVITDEYKVPSKKFKVLGNTDRQSIKKNLIGALL | 10 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINESGVDA | |
| | KAILSAKLSKSRRLENLIAQLPGEKKNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVLKQLKRRRYTGWGRLSKKLINGIRDKQSG | |
| | KTILDFLKSDGFANRNFMQLIHDDSLTFKEEIQKAQVSGQGDSLHEQIANLA | |
| | GSPAIKKGILQTVKIVDEIVKVMGRHKPENIVIEMARENQTTQKGQKNSRER | |
| | MKRLEEGIKELGSQILKEHPVENTQLQNEKLYLYYLQNGKDMYTGEELDIDR | |
| | LSDYDVDHIIPQSFIKDDSIDNKVLTSSDEARGKSDNVPSEEVVKKMKSYWR | |
| | QLLKAKLISQRKFDNLTKAERGGLSELDKAGFIKRQLVETRQITKHVAQILD | |
| | SRFNTERDENDKLIRDVKIITLKSKLVSDFRKDFQFYKVREINDYHHAHDAY | |
| | LNAVVGTALIKKYPKLESEFVYGDYKVYDVRKMIAKSEQEIGKATAKRFFYS | |
| | NIMNFFKTEIKLANGEIRKRPLIETNEETGEIVWDKEKDFATVRKVLSMPQV | |
| | NIVKKTEVQTGGFSKESILPKRESDKLIARKKDWDTKKYGGFDSPTVAYSVL | |
| | VVAKVEKGKTKKLKTVKELVGITIMERSSFEKDPIDFLEAKGYKEVQKDLII | |
| | KLPKYSLFELENGRKRLLASAGELQKGNELALPSKYVTFLYLASHYEKLKGS | |
| | PEDEEQKQLYVEQHKHYFDEIVEQISEFSKRYILADKNLDKILSLYNKHRDK | |
| | SISEQAESIINLFTLTALGAPAAFKFFDTTIDRKRYTSTKEILNSTLIHQSI | |
| | TGLYETRIDLSQLGGD | |
| CasEnd-51 | MDKKYSIGLDIGTNSVGWAVITDEYKVPSKKFKVLGNTDRQSIKKNLLGALL | 11 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKELADSTEKADLRLIYL | |
| | ALAHMIKFRGHFLIEGDLNSDNSDVDKLFIQLVQTYNQLFEENPINEEGVDA | |
| | KAILSAKLSKSRRLENLIAQLPGEKKNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEDFYKFIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKEEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYAHLFDDKVMKQLKRRRYTGWGRLSRKLINGIRDKQSG | |
| | KTILDFLKSDGFANRNFMQLIHDDSLTFKEEIKKAQVSGQGDSLHEQIANLA | |
| | GSPAIKKGILQTVKIVDELVKVMGRHKPENIVIEMARENQTTQKGQKNSRER | |
| | MKRLEEGIKELGSQILKEHPVENTQLQNEKLYLYYLQNGKDMYTGEELDIDR | |
| | LSDYDVDHIVPQSFIKDDSIDNKVLTSSKEARGKSDNVPSEEVVKKMKNYWR | |
| | QLLKAKLISQRKFDNLTKAERGGLSELDKAGFIKRQLVETRQITKHVAQILD | |
| | SRMNTERDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEIKLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKDPVAFLEKKGYKEVQKDLII | |
| | KLPKYSLFELENGRKRLLASAGELQKGNELALPSKYVTFLYLASHYEKLKGS | |
| | PEDNEQKQLYVEQHKHYLDEILDQISEFSKRYILADKNLEKILSLYNKNEDK | |
| | SIEEQAENIINLFTLTNLGAPAAFKFFDTTIDRKRYTSTKEILDATLIHQSI | |
| | TGLYETRIDLSQLGGD | |
| CasEnd-52 | MDKKYSIGLDIGTNSVGWAVITDEYKVPSKKFKVLGNTDRQSIKKNLLGALL | 12 |
| | FDSGETAEATRLKRTARRRYTRRKNRIRYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINESGVDA | |
| | KAILSAKLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKEEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYANLFDDKVMKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQTVKIVDEIVKVMGRHKPENIVIEMARENQTTQKGQKNSRER | |
| | MKRLEEGIKELGSQILKEHPVENTQLQNEKLYLYYLQNGKDMYTGDELDIDR | |
| | LSDYDVDHIIPQSFIKDDSIDNKVLTSSEKARGKSDNVPSEEVVKKMKNYWR | |
| | QLLNAKLISQRKFDNLTKAERGGLSELDKAGFIKRQLVETRQITKHVAQILD | |
| | SRMNTEYDENDKLIRDVKIITLKSKLVSDFRKDFQFYKVREINDYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKDPVDFLEAKGYKEIQKDLII | |
| | KLPKYSLFELENGRKRLLASAGELQKGNELALPSKYVTFLYLASHYEKLKGS | |
| | PEDNEQKQLFVEQHRHYFDEILEQISEFSERYILADKNLDKVLSLYNNFRDK | |
| | SIEEQAENIINLFTLTNLGAPAAFKFFDTTIDRKRYTSTKEVLDSTLIHQSI | |
| | TGLYETRIDLSQLGGD | |
| CasEnd-53 | MDKKYSIGLDIGTNSVGWAVITDEYKVPSKKFKVLGNTDRHSIKKNLIGALL | 13 |
| | FDSGETAEATRLKRTARRRYTRRKNRICYLQEIFSNEMAKVDDSFFQRLEES | |
| | FLVEEDKKNERHPIFGNIADEVAYHEKYPTIYHLRKKLADSTEKADLRLIYL | |
| | ALAHMIKFRGHFLIEGDLNSDNSDVQKLFIQLVQTYNQLFEESPLNEEGVDA | |
| | KAILTEKLSKSRRLENLIAQFPGEKKNGLFGNLIALSLGLTPNFKSNEDLAE | |
| | DAKLQLSKDTYDEDLENLLGQIGDQYADLFLAAKNLYDAILLSDILTVNDEI | |
| | TKAPLSASMVKRYDEHHQDLKLLKKFVRQQLPEKYKEIFSDKSKNGYAGYID | |
| | GKTSQEEFYKYLKPILEKMDGSEEFLEKIEREDFLRKQRTFDNGSIPHQIHL | |
| | QELHAILRRQEKYYPFLKENREKIEKILTFRIPYYVGPLARGNSRFAWMSRK | |
| | SDEKITPWNFDEVVDKEASAQKFIERMTNNDLYLPDEKVLPKHSLLYEKFTV | |
| | YNELTKVKYVTEGMRKPEFLSAEQKQEIVDLLFKKNRKVTKKKLKEYYFKEF | |
| | ECFDSVEITGVDDRFNASLGTYHDLLKIIKDKDFLDNEENEKILEDIVLTLT | |
| | LFEDREMIKERLEKYANLFDKKQLKQLKRRHYTGWGRLSAKLINGIRDKQSG | |
| | KTILDYLIDDGFANRNFMQLIHDDSLTFKEEIQKAQVSGDGDSLHEHIANLA | |
| | GSPAIKKGILQTVKVVDELVKVMGRHKPENIVIEMARENQTTQKGQRNSRQR | |
| | LKRLEEAIKELGSKILKEHPVENSQLQNDRLYLYYLQNGKDMYTGEELDIDR | |
| | LSQYDIDHIIPQSFIKDDSIDNRVLVSSAKARGKSDNVPSEEVVKKMKNYWK | |
| | QLLDAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-54 | MKKKYSIGLDIGTNSVGWAVITDEYKVPSKKFKVLGNTDRHSIKKNLIGALL | 14 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKELVDSTEKADLRLIYL | |
| | ALAHMIKFRGHFLIEGDLNSENSDVQKLFIQLVQTYNQLFEENPLNEEGVDA | |
| | KAILSAKLSKSRRLENLIAQFPGEKKNGLFGNLIALSLGLTPNFKSNEDLAE | |
| | DAKLQLSKDTYDDDLENLLGQIGDQYADLFLAAKNLSDAILLSDILRVNTEI | |
| | TKAPLSASMIKRYDEHHQDLTLLKALVRKQLPEKYKEIFFDKSKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMGKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDKKVMKQLKRRRYTGWGRLSRKLINGIRDKQSG | |
| | KTILDFLKSDGFANRNFMQLIHDDSLTFKEEIEKAQVSGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGEELDINR | |
| | LSDYDVDHIVPQSFLKDDSIDNKVLTRSKKARGKSDNVPSEEVVKKMKSYWR | |
| | QLLKAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEIKLANGEIRKRPLIETNEETGEIVWDKERDFATVRKVLSMPQV | |
| | NIVKKTEVQTGGFSKESILPKRNSDKLIARKKDWDPKKYGGFDSPTVAYSVL | |
| | VVAKIEKGKSKKLKTVKELLGITIMERSSFEKDPVAFLEAKGYKEVKKDLII | |
| | KLPKYSLFELENGRKRMLASAGELQKGNELALPSKYVNFLYLASHYEKLKGS | |
| | PEDNEQKLLYVEQHKHYLDEIIDQISEFSKRVILADKNLEKVLSAYNEHRDK | |
| | SIEEQAENIIHLFTLTNLGAPAAFKYFGTTIDRKRYTSTKEVLDATLIHQSI | |
| | TGLYETRIDLSQLGGD | |
| CasEnd-55 | MDKKYSIGLDIGTNSVGWAVITDEYKVPSKKFKVLGNTDRHSIKKNLIGALL | 15 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSDEKADLRLIYL | |
| | ALAHMIKFRGHFLIEGDLNSDNSDVQKLFIQLVQTYNQLFEENPINAEGVDA | |
| | KAILSARLSKSRRLENLIAQLPGEKKNGLFGNLIALSLGLTPNFKSNEDLSE | |
| | DAKLQLSKDTYDDDLENLLGQIGDQYADLFLAAKNLSDAILLSDILRVNTEI | |
| | TKAPLSASMIKRYDEHHQDLTLLKDLVRDQLPEKYKEIFFDQSKNGYAGYID | |
| | GGASQEEFYKYIKPILEKMDGTEELLEKLNREDLLRKQRTFDNGSIPHQIHL | |
| | GELHAILRRQEEYYPFLKENREKIEKILTFRIPYYVGPLARGNSRFAWMTRK | |
| | SDETITPWNFEEVVDKGASAQKFIERMTNFDKNLPNEKVLPKHSLLYEYFTV | |
| | YNELTKVKYVTEGMRKPEFLSGEQKKEIVDLLFKKNRKVTVKQLKEYYFKKF | |
| | ECFDSVEISGVEDRFNASLGTYHDLLKIIKDKDFLDNEENEKILEDIVLTLT | |
| | LFEDREMIKERLKKYANLFDKKVMKQLKRRRYTGWGRLSRKLINGIRDKQSG | |
| | KTILDFLKEDGFANRNFMQLIHDDSLTFKEEIEKAQVIGKGESLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELLGITIMERSAFEKNPVAFLEAKGYQEVKKDLII | |
| | KLPKYSLFELENGRKRMLASAGELQKGNELALPSKYVNFLYLASHYEKLKGS | |
| | PEDNEQKQLFVEQHKHYLDEIIDQISEFSKRVILADANLEKVKSAYEKHRDK | |
| | SIEEQAENIIHLFTLTALGAPAAFKYFDTTIDRKRYTSTKEVLNATLIHQSI | |
| | TGLYETRIDLSQLGGD | |
| CasEnd-56 | MDKKYSIGLDIGTNSVGWAVITDEYKVPSKKFKVLGNTDRHSIKKNLIGALL | 16 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENSDVQKLFIQLVQTYNQLFEENPINEEGVDA | |
| | KAILSAKLSKSRRLENLIAQFPGEKKNGLFGNLIALSLGLTPNFKSNEDLSE | |
| | DAKLQLSKDTYDEDLDNLLAQIGDQYADLFLAAKNLSDAILLSDILTVNDEI | |
| | TKAPLSASMIKRYDEHHQDLTLLKALVRQQLPEKYKEIFFDESKNGYAGYID | |
| | GGASQEEFYKFIKPILEKMDGTEELLAKINREDFLRKQRTFDNGSIPHQIHL | |
| | GELHAILRRQEEFYPFLKENREKIEKILTFRIPYYVGPLARGNSRFAWMTRK | |
| | SDETITPWNFEEVVDKGASAQAFIERMTNFDKNLPTEKVLPKHSLLYEYFTV | |
| | YNELTKVKYVTEGMGKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDKKVLKQLKRRRYTGWGRLSKKLINGIRDKQSG | |
| | KTILDFLKSDGFANRNFMQLIHDDSLTFKEAIKKAQVSGQGDSLHEHIANLA | |
| | GSPAIKKGILQTVKVVDELVKVMGRHKPENIVIEMARENQTTQKGQKNSRER | |
| | MKRIEEAIKELGSQILKEHPVENTQLQNDKLYLYYLQNGKDMYTGEELDIDR | |
| | LSDYDVDHIIPQSFIKDDSIDNKVLTSSKKARGKSDDVPSEEVVKKMKNYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRESDKLIARKKDWDTKKYGGFDSPTVAYSVL | |
| | VVAKVEKGKAKKLKTVKELVGITIMERSSFEKDPVAFLEDKGYKDIQKDLII | |
| | KLPKYSLFELENGRKRLLASAGELQKGNELALPQKYVTFLYHASHYEKLKGS | |
| | PEDNEKKLLYVEQHKNYFDEILDQISEFSKRYILADKNLEKIKSLYNENEDY | |
| | SIEELAESFINLFTLTALGAPAAFKFFDTTIDRKRYTSTKEILDATLIHQSI | |
| | TGLYETRIDLSQLGGD | |
| CasEnd-57 | MDKKYSIGLDIGTNSVGWAVITDEYKVPSKKFKVLGNTDRHSIKKNLIGALL | 17 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKELADSTEKADLRLIYL | |
| | ALAHIIKFRGHFLIEGDLNSDNSDVQKLFIQLVQTYNQLFEENPLNESGVDA | |
| | KAILSAKLSKSRRLENLIAQFPGEKRNGLFGNLIALSLGLTPNFKSNEDLAE | |
| | DAKLQLSKDTYDEDLDNLLAQIGDQYADLFLAAKNLSDAILLSGILTVNDEI | |
| | TKAPLSASMIKRYDEHHQDLTLLKALVRQQLPEKYKEIFFDESKNGYAGYID | |
| | GGASQEEFYKYIKPILEKMDGTEELLAKLNREDFLRKQRTFDNGSIPHQIHL | |
| | GELHAILRRQEDYYPFLKENREKIEKILTFRIPYYVGPLARGNSRFAWMTRK | |
| | SDETITPWNFEEVVDKEASAQAFIERMTNFDKNLPTEKVLPKHSLLYEYFTV | |
| | YNELTKVKYVTEGMGKPEFLSGEQKQEIVDLLFKKNRKVTVKQLKEDLFKEI | |
| | ECFDSVEISGVEDRFNASLGTYHDLLKIIKDKDFLDNEENEDILEDIVLTLT | |
| | LFEDREMIEERLKKYADLFDDKVLKKLKRRRYTGWGRLSRKLINGIRDKQSG | |
| | KTILDFLKSDGFANRNFMQLIHDDSLTFKEEIQKAQVKGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNDKLYLYYLQNGKDMYTGEELDIDR | |
| | LSDYDVDHIIPQSFIKDDSIDNKVLTSSKKARGKSDDVPSEEVVKKMKSYWR | |
| | QLLKAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRESDKLIARKKDWDTKKYGGFDSPTVAYSVL | |
| | VVAKVEKGKSKKLKTVKELVGITIMERSSFEKNPIDFLEAKGYKNVQKDLII | |
| | KLPKYSLFELENGRKRLLASAGELQKGNELALPSKYVTFLYLASHYEKLKGS | |
| | PEDNEQKQLFVEQHKHYFDEILDQISEFSERYILADKNLDKILSLYNENRDY | |
| | SIEEQAENFINLFTLTNLGAPAAFKFFGTTIDRKRYTSTKEILDATLIHQSI | |
| | TGLYETRIDLSQLGGD | |
| CasEnd-58 | MDKKYSIGLDIGTNSVGWAVITDEYKVPSKKFKVLGNTDRHSIKKNLIGALL | 18 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSDEKADLRLIYL | |
| | ALAHMIKFRGHFLIEGDLNSDNSDVQKLFIQLVQTYNQLFEENPINEEGVDA | |
| | KAILSARLSKSRRLENLIAQFPGEKKNGLFGNLIALSLGLIPNFKSNEDLAE | |
| | DAKLQLSKDTYDDDLENLLGQIGDQYADLFLAAKNLSDAILLSDILTVNSEI | |
| | TKAPLSASMVKRYDEHHQDLTLLKALVRQQLPEKYKEIFFDQSKNGYAGYID | |
| | GGASQEEFYKYIKPILEKMDGTEEFLAKINREDFLRKQRTFDNGSIPHQIHL | |
| | GELHAILRRQEKYYPFLKENREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVLKQLKRRRYTGWGRLSRKLINGIRDKQSG | |
| | KTILDFLKSDGFANRNFMQLIHDDSLTFKEEIEKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSSFEKNPIAFLEAKGYKEVKKDLII | |
| | KLPKYSLFELENGRKRLLASAGELQKGNELALPSKYVTLLYLASHYEKLKGS | |
| | PEDNEQKQLFVEQHKHYFDEIIDQISEFSKRYILADANLEKIKSLYEKNRDK | |
| | SIEEQAENFIHLLTFTALGAPAAFKFFDTTIDRKRYTSTKEILNATLIHQSI | |
| | TGLYETRIDLSQLGGD | |
| CasEnd-59 | MKKKYSIGLDIGTNSVGWAVITDEYKVPSKKFKVLGNTDRQSIKKNLLGALL | 19 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINEEGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKQAIVDLLFKKNRKVTVKQLKEDYFKKI | |
| | ECFDSVEISGVEDRFNASLGTYHDLLKIIKDKEFLDNEENEDILEDIILTLT | |
| | LFEDKEMIEERLKKYANLFDDKVLKQLKRRRYTGWGRLSRKLINGIRDKQSG | |
| | KTILDFLKSDGFANRNFMQLIHDDSLTFKEEIKKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTSSKKARGKSDNVPSEEVVKKMKNYWR | |
| | QLLNAKLISQRKFDNLTKAERGGLSELDKAGFIKRQLVETRQITKHVAQILD | |
| | SRMNTKRDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRESDKLIARKKDWDPKKYGGFDSPTVAYSVL | |
| | VVAKVEKGKAKKLKSVKELVGITIMERSSFEKDPVAFLEAKGYKEIQKDLII | |
| | KLPKYSLFELENGRKRMLASAGELQKGNELALPSKYVTFLYLASHYEKLKGS | |
| | PEDNEQKQLFVEQHKHYLDEILDQISEFSERYILADKNLDKILSLYNKHRDK | |
| | SIEEQAENIINLFTLTNLGAPAAFKFFDTTIDRKRYTSTKEILNATLIHQSI | |
| | TGLYETRIDLSQLGGD | |
| CasEnd-60 | MDKKYSIGLDIGTNSVGWAVITDEYKVPSKKFKVLGNTDRQSIKKNLLGALL | 20 |
| | FDSGETAEATRLKRTARRRYTRRKNRIRYLQEIFANEMAKVDDSFFHRLEES | |
| | FLVEEDKRHERHPIFGNIVDEVAYHEKYPTIYHLRKELADSTEKADLRLIYL | |
| | ALAHMIKFRGHFLIEGDLNSDNSDVDKLFIQLVQTYNQLFEENPINESGVDA | |
| | KAILSAKLSKSRRLENLIAQLPGEKKNGLFGNLIALSLGLTPNFKSNEDLSE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYANLFDDKVLKQLKRRRYTGWGRLSKKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRLEEGLKELGSQILKEHPVENTQLQNDKLYLYYLQNGKDMYTGQELDIDR | |
| | LSDYDVDHIIPQSFIKDDSIDNKVLTSSDKARGKSDNVPSEEVVKKMKSYWR | |
| | QLLNAKLISQRKFDNLTKAERGGLSELDKAGFIKRQLVETRQITKHVAQILD | |
| | SRFNTERDENDKLIREVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEIKLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKTKKLKTVKELVGITIMERSSFEKDPVAFLEKKGYKEIQKDLII | |
| | KLPKYSLFELENGRKRMLASAGELQKGNELALPSKYVTFLYLASHYEKLKGS | |
| | PEDNEQKQLFVEQHKDYFDEIIEQISEFSKRYILADKNLEKILSLYNKNSDK | |
| | PIEEQAESIINLFTLTNLGAPAAFKFFDTTIDRKRYTSTKEILDATLIHQSI | |
| | TGLYETRIDLSQLGGD | |
| CasEnd-61 | MDKKYSIGLDIGTNSVGWAVITDEYKVPSKKFKVLGNTDRHSIKKNLLGALL | 21 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKYLADSPEKADLRLIYL | |
| | ALAHIIKFRGHFLIEGDLNSDNSDVQKLFIQLVQTYNQLFEESPLNAEGVDA | |
| | KAILSARLSKSRRLENLIAQIPGEKKNGLFGNLIALSLGLTPNFKSNEDLAE | |
| | DAKLQLSKDTYDDDLENLLAQIGDQYADLFLAAKNLSDAILLSGILTVNDEI | |
| | TKAPLSASMIKRYDEHHQDLTLLKALIRQQLPEKYKEIFFDQSKNGYAGYID | |
| | GGASQEEFYKYLKPILEKLDGSEEFLAKIEREDFLRKQRTFDNGSIPHQIHL | |
| | GELHAILRRQEKYYPFLKDNREKIEKILTFRIPYYVGPLARGNSRFAWMTRK | |
| | SDETITPWNFEEVVDKEASAQAFIERMTNFDKNLPNEKVLPKHSLLYEKFTV | |
| | YNELTKVKYVTEGMRKPEFLSAEQKQEIVDLLFKKNRKVTVKQLKEFYFKEI | |
| | ECFDIVEISGVEDRFNASLGTYHDLLKIIKDKDFLDNEENEDILEDIVLTLT | |
| | LFEDREMIEERLKKYANLFDKKVLKKLKRRRYTGWGRLSAKLINGIRDKQSG | |
| | KTILDFLKADGFANRNFMQLIHDDSLTFKEEIKKAQVIGQGESLHELIANLA | |
| | GSPAIKKGILQTIKIVDELVKVMGRHKPENIVIEMARENQTTQKGQKNSRER | |
| | MKRIEEAIKELGSQILKEHPVENTQLQNEKLYLYYLQNGKDMYTGEELDIDR | |
| | LSDYDVDHIVPQSFLKDDSIDNKVLVSSEKARGKSDNVPSEEVVKKMKSYWS | |
| | KLLDAKLITQRKFDNLTKAERGGLTELDKAGFIKRQLVETRQITKHVAQILD | |
| | SRFNTKYDENDKLIRDVKIITLKSKLVSDFRKDFQLYKVREINNYHHAHDAY | |
| | LNAVVGTALIKKYPKLESEFVYGDYKVYDVRKMIAKSEQEIGKATAKMFFYS | |
| | NIMNFFKSEISLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VIAKVEKGKAKKLKTVKELVGITIMERSSFEKNPIAFLEDKGYKEIKKDLII | |
| | KLPKYSLFELENGRKRLLASAGELQKGNELALPNKYVTFLYLASHYEKLKGS | |
| | EEDNEQKQNFVEQHKHYFDEIIEQISEFSKRYILADANLEKILSLYEKNRDL | |
| | SIEEQAESFINLFTFTALGAPAAFKFFDTTIDRKRYTSTKEVLNATLIHQSI | |
| | TGLYETRIDLSQLGGD | |
| CasEnd-62 | MKKKYSIGLDIGTNSVGWAVITDDYKVPSKKFKVLGNTDRQSIKKNLIGALL | 22 |
| | FDSGETAEATRLKRTARRRYTRRKNRIRYLQEIFAEEMAKVDDSFFQRLEES | |
| | FLVEEDKKHERHPIFGNIVDEVAYHEKYPTIYHLRKKLVDSTEKADLRLIYL | |
| | ALAHMIKFRGHFLIEGDLNSENSDVQKLFIQLVQTYNQLFEENPINESGVDA | |
| | KAILSARLSKSRRLENLIAQIPGEKKNGLFGNLIALSLGLTPNFKSNFDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SDETITPWNFEEVVDKGASAQSFIERMTNFDENLPNEKVLPKHSLLYEYFTV | |
| | YNELTKVKYVTEGMGKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVMKQLKRRRYTGWGRLSRKLINGIRDKQSG | |
| | KTILDFLKSDGFANRNFMQLIHDDSLTFKEEIKKAQVKGQGDSLHEQIANLA | |
| | GSPAIKKGILQTVKVVDELVKVMGRHKPENIVIEMARENQTTQKGQKNSRER | |
| | MKRIEEGIKELGSQILKEHPVENAQLQNDKLYLYYLQNGRDMYTGEELDINR | |
| | LSQYDVDHIVPQSFLKDDSIDNKVLTRSEKARGKSDNVPSEEVVKKMKSYWR | |
| | QLLNAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKKYDVRKMIAKSEQEIGKATAKYFFYS | |
| | NIMNFFKSEIKLANGEIRKRPLIETNEETGEIVWDKERDFATVRKVLSMPQV | |
| | NIVKKTEVQTGGFSKESILPKRESDKLIARKKGWDPKKYGGFDSPTVAYSVL | |
| | VVAKVEKGKSKKLKTVKELLGITIMERSSFEKDPVDFLEAKGYKEVKKDLII | |
| | KLPKYSLFELENGRKRMLASAGELQKGNELALPSKYVNFLYLASHYEKLKGK | |
| | PEDNEQKQLYVEQHKHYLDEIIDQISEFSKRVILADKNLEKVLSAYNNHRDK | |
| | SIREQAENIIHLFTLTNLGAPAAFKYFDTTIDRKRYTSTKEVLDATLIHQSI | |
| | TGLYETRIDLSQLGGD | |
| CasEnd-63 | MDKKYSIGLDIGTNSVGWAVITDEYKVPSKKFKVLGNTDRHSIKKNLIGALL | 23 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVDKLFIQLVQTYNQLFEENPINESGVDA | |
| | KAILSAKLSKSRRLENLIAQFPGEKRNGLFGNLIALSLGLTPNFKSNFDLAE | |
| | DAKLQLSKDTYDEDLENLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKYIKPILEKMDGTEEFLAKINREDFLRKQRTFDNGSIPHQIHL | |
| | GELHAILRRQEEYYPFLKENREKIEKILTFRIPYYVGPLARGNSRFAWMTRK | |
| | SDETITPWNFEEVVDKGASAEAFIERMTNFDKNLPSEKVLPKHSLLYEYFTV | |
| | YNELTKVKYVTEGMGKPHFFDGNVKQEIVDLLFKKDRKVTKKKLLDFLFKEI | |
| | DEFRIVDISGVEDAFNASLGTYHDLLKIIKDKDFLDNEENEDILEDIVLTLT | |
| | LFEDKEMIEERLKKYANLFDKKVLKQLKRRRYTGWGRLSRKLINGIRDKQSG | |
| | KTILDFLKSDGFANRNFMQLIHDDSLTFKEAIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-64 | MDKKYSIGLDIGTNSVGWAVITDEYKVPSKKFKVLGNTDRHSIKKNLIGALL | 24 |
| | FDSGETAEATRLKRTARRRYTRRKNRICYLQEIFSNEMAKVDDSFFHRLEES | |
| | FLVEEDKRNERHPIFGNIVEEVAYHEKYPTIYHLRKHLADSDEKADLRLVYL | |
| | ALAHIIKFRGHFLIEGDLNAENSDVQKLFKELLEAYNQTFEESPLEEITVDA | |
| | EAILTEKLSKSRRLENLIAEFPGEKKNGLFGNLVALSLGLTPNFKSNEDLSE | |
| | DAKLQFSKDTYDEDLEELLGQIGDEYADLFVAAKNLYDAILLSGILTVKDSS | |
| | TKAPLSASMVKRYDEHHQDLTLLKNFVRKQLPEKYKEIFFDQSKNGYAGYID | |
| | GGASQEEFYKYLKKLLEKIDGSEYFLDKIEREDFLRKQRTFDNGSIPHQIHL | |
| | QELKAIIRRQEKYYPFLKENQEKIEKILTFRIPYYVGPLARGNSRFAWASRK | |
| | SDETITPWNFDEVVDKEKSAEKFIERMTNFDKNLPEEKVLPKHSLLYEKFTV | |
| | YNELTKVKYVTEGGKKPEFFDANMKQEIFDNVFKKYRKVTKKQLLDYLAKEF | |
| | DEFRIVDISGVEDRFNASLGTYHDLKKILGDKDFLDNDDNEKILEDIIKTLT | |
| | LFEDREMIKKRLEKYSDLFDKKQLKKLERRRYTGWGRLSAKLINGIRDKESG | |
| | KTILDYLIDDGFTNRNFMQLIHDDNLTFKEEIAKAQVIGKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-65 | MDKKYSIGLDIGTNSVGWAVITDEYKVPSKKFKVLGNTDRHSIKKNLIGALL | 25 |
| | FDSGETAEATRLKRTARRRYTRRKNRIRYLQEIFAEEMAKVDDSFFHRLEES | |
| | FLVEEDKKNERHPIFGNIVDEVAYHEKYPTIYHLRKKLADSDEKADLRLIYL | |
| | ALAHIIKFRGHFLIEGDLNSDNSDVQKLFIQLVQTYNQLFEESPINEEGVDA | |
| | KAILTAKLSKSRRLENLIAQFPGEKKNGLFGNLIALSLGLTPNFKSNFDLAE | |
| | DAKLQFSKDTYDDDLENLLGQIGDQYADLFLAAKNLSDAILLSDILTVNSEI | |
| | TKAPLSASMVKRYDEHHQDLTLLKKLVRQQLPEKYKEIFFDQSKNGYAGYID | |
| | GGASQEEFYKYIKPILEKVDGSEELLAKIEREDFLRKQRTFDNGSIPHQIHL | |
| | GELHAILRRQEKYYPFLKENREKIEKILTFRIPYYVGPLARGNSRFAWMSRK | |
| | SEETITPWNFEEVVDKEASAQSFIERMTNFDKNLPNEKVLPKHSLLYEKFTV | |
| | YNELTKVKYVTEGMRKPEFLSAEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDKKVLKQLKRRRYTGWGRLSRKLINGIRDKQSG | |
| | KTILDFLIDDGFANRNFMQLIHDDSLTFKEEIEKAQVIGQGESLHELIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQLYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKNPIAFLEAKGYKEVKKDLII | |
| | KLPKYSLFELENGRKRLLASAGELQKGNELALPNKYVTLLYLASHYEKLKGS | |
| | PEDNEEKQLFVEQHKHYFDEIIEQISEFSKRYILADANLEKILSLYEKNRDK | |
| | SIEEQAENFIHLFTFTALGAPAAFKFFDTTIDRKRYTSTKEILNATLIHQSI | |
| | TGLYETRIDLSQLGGD | |
| CasEnd-66 | MDKKYSIGLDIGTNSVGWAVITDEYKVPSKKFKVLGNTDRHSIKKNLIGALL | 26 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEEDPINESGVDA | |
| | KAILSAKLSKSRRLENLIAQFPGEKKNGLFGNLIALSLGLTPNFKSNEDLAE | |
| | DAKLQLSKDTYDEDLENLLAQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKYIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVLKQLKRRRYTGWGRLSKKLINGIRDKQSG | |
| | KTILDFLKSDGFANRNFMQLIHDDSLTFKEEIKKAQVKGQGDSLHEHIANLA | |
| | GSPAIKKGILQTVKVVDELVKVMGRHKPENIVIEMARENQTTQKGQKNSRER | |
| | MKRLEEGIKELGSQILKEHPVENTQLQNDKLYLYYLQNGKDMYTGDELDIDR | |
| | LSDYDVDHIIPQSFIKDDSIDNKVLTSSDKARGKSDNVPSEEVVKKMKSYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEAKGYKNIQKDLII | |
| | KLPKYSLFELENGRKRLLASAGELQKGNELALPSKYVTFLYLASHYEKLKGS | |
| | PEDNEKKRLYVEQHKHYFDEIVDQISEFSKRYILADKNLEKILSLYNNNRDK | |
| | SINEQAENFINLFTLTALGAPAAFKFFDTTIDRKRYTSTKEILNSTLIHQSI | |
| | TGLYETRIDLSQLGGD | |
| CasEnd-67 | MDKKYSIGLDIGTNSVGWAVITDEYKVPSKKFKVLGNTDRHSIKKNLIGALL | 27 |
| | FDSGETAEATRLKRTARRRYTRRKNRICYLQEIFSNEMAKVDDSFFHRLEES | |
| | FLVEEDKKNERHPIFGNIVDEVAYHKKYPTIYHLRKKLADSDEKADLRLIYL | |
| | ALAHMIKFRGHFLIEGDLNSDNSDVQKLFIQLVQTYNQLFEENPINEEGVDA | |
| | KAILSERLSKSRRLENLIAQFPGEKKNGLFGNLIALSLGLTPNFKSNFDLAE | |
| | DAKLQLSKDTYDEDLENLLGQIGDQYADLFLAAKNLSDAILLSDILTVNDEI | |
| | TKAPLSASMVKRYDEHHKDLKLLKELVRQQLPEKYKEIFSDKSKNGYAGYID | |
| | GKTSQEEFYKYIKPILEKVDGSEEFLDKINREDFLRKQRTFDNGSIPHQIHL | |
| | GELHAILRRQEKYYPFLKENREKIEKILTFRIPYYVGPLARGNSRFAWMTRK | |
| | SDEKITPWNFDEVVDKEASAQKFIERMTNNDLYLPDEKVLPKHSLLYEYFTV | |
| | YNELTKVKYVTEGMRKPEFLSGEQKQEIVDLLFKKNRKVTVKKLKEYYFKKI | |
| | ECFDSVEISGVDDRFNASLGTYHDLLKIIKDKDFLDNEENEDILEDIVLTLT | |
| | LFEDREMIKKRLEKYADLFDKKQLKQLKRRHYTGWGRLSRKLINGIRDKQSG | |
| | KTILDFLISDGFANRNFMQLIHDDSLTFKEEIQKAQVIGDGDSLHEHIANLA | |
| | GSPAIKKGILQTVKVVDELVKVMGRHKPENIVIEMARENQTTQKGQRNSRQR | |
| | LKRLEEGIKELGSNILKENPVENTQLQNDRLYLYYLQNGKDMYTGEELDIDR | |
| | LSQYDIDHIIPQSFIKDDSIDNKVLVSSAEARGKSDNVPSEEVVKKMKGYWR | |
| | KLLEAGLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-68 | MDKKYSIGLDIGTNSVGWAVITDEYKVPSKKFKVLGNTDRHSIKKNLIGALL | 28 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVQKLFIQLVQTYNQLFEENPINEEGVDA | |
| | KAILSAKLSKSRRLENLIAQLPGEKRNGLFGNLIALSLGLTPNFKSNFDLAE | |
| | DAKLQLSKDTYDEDLENLLGQIGDQYADLFLAAKNLSDAILLSDILTVNSEI | |
| | TKAPLSASMIKRYDEHHQDLTLLKALVREQLPEKYKEIFFDETKNGYAGYID | |
| | GGASQEEFYKYIKPILEKMDGTEEFLAKINREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMGKPEFFSGEQKQEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIEERLKKYADLFDDKVLKQLKRRRYTGWGRLSKKLINGIRDKQSG | |
| | KTILDFLKSDGFANRNFMQLIHDDSLTFKEEIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTSSEKARGKSDDVPSEEVVKKMKSYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRESDKLIARKKDWDTKKYGGFDSPTVAYSVL | |
| | VVAKVEKGKKKKLKTVKELVGITIMERSSFEKDPIAFLEAKGYKEVQEDLII | |
| | KLPKYSLFELENGRKRLLASAGELQKGNELALPNKYVTFLYLASHYEKLKGS | |
| | PEDEEQHQLYVEQHKHYFDEIFDQISEFSERYILADKNLDKIKSLYNKNRDK | |
| | SISEQAESFINLFTLTALGAPAAFKFFGTTIDRKRYTSTKEILNATLIHQSI | |
| | TGLYETRIDLSQLGGD | |
| CasEnd-69 | MKKKYSIGLDIGTNSVGWAVITDEYKVPSKKFKVLGNTDRQSIKKNLLGALL | 29 |
| | FDSGETAEATRLKRTARRRYTRRKNRIRYLQEIFANEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINESGVDA | |
| | KAILSARLSKSRRLENLIAQLPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKYIKPILEKMDGTEELLAKINREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKGASAQAFIERMTNFDKNLPNEKVLPKHSLLYEYFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYAHLFDDKVLKQLKRRRYTGWGRLSRKLINGIRDKQSG | |
| | KTILDFLKSDGFANRNFMQLIHDDSLTFKEDIQKAQVSGQGDSLHEQIANLA | |
| | GSPAIKKGILQTVKIVDELVKVMGRHKPENIVIEMARENQTTQKGQKNSRER | |
| | MKRLEEGIKELGSQILKEHPVENTQLQNEKLYLYYLQNGKDMYTGEELDIDR | |
| | LSDYDVDHIIPQSFIKDDSIDNKVLTSSKKARGKSDNVPSEEVVKKMKSYWR | |
| | QLLNAKLISQRKFDNLTKAERGGLSELDKAGFIKRQLVETRQITKHVAQILD | |
| | SRFNTEYDENDKLIREVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKTKKLKTVKELVGITIMERSSFEKDPVDFLEAKGYKEIQKDLII | |
| | KLPKYSLFELENGRKRMLASAGELQKGNELALPSKYVTFLYLASHYEKLKGS | |
| | PEDNEQKQLYVEQHKHYFDEIVDQISEFSKRYILADANLDKILSLYNKNRDK | |
| | SIREQAENIINLETLTALGAPAAFKFFDTTIDRKRYTSTKEILDATLIHQSI | |
| | TGLYETRIDLSQLGGD | |
| CasEnd-70 | MKKKYSIGLDIGTNSVGWAVITDDYKVPSKKFKVLGNTDRQSIKKNLIGALL | 30 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNPENSDVDKLFIQLVQTYNQLFEENPINESGVDA | |
| | KAILSAKLSKSRRLENLIAQLPGEKKNGLFGNLIALSLGLTPNFKSNFDLAE | |
| | DAKLQLSKDTYDDDLENLLAQIGDQYADLFLAAKNLSDAILLSDILRVNTEI | |
| | TKAPLSASMIKRYDEHHQDLTLLKALVRQQLPEKYKEIFFDPSKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNEDENLPNEKVLPKHSLLYEYFTV | |
| | YNELTKVKYVTEGMGKPEFLSGEQKKEIVDLLFKKNRKVTVKQLKEDYFKEI | |
| | ECFDSVEISGVEDRFNASLGTYHDLLKIIKDKDFLDNEENEDILEDIVLTLT | |
| | LFEDREMIEERLKKYANLFDKKVMKQLKRRRYTGWGRLSRKLINGIRDKQSG | |
| | KTILDFLKSDGFANRNFMQLIHDDSLTFKEAIKKAQVSGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGEELDINR | |
| | LSDYDVDHIVPQSFLKDDSIDNKVLTRSDKARGKSDNVPSEEVVKKMKNYWR | |
| | QLLKAGLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKKYDVRKMIAKSEQEIGKATAKYFFYS | |
| | NIMNFFKTEITLANGEIRKRPLIETNEETGEIVWDKERDFATVRKVLSMPQV | |
| | NIVKKTEVQTGGFSKESILPKRESDKLIARKKGWDPKKYGGFDSPTVAYSVL | |
| | VVAKVEKGKTKKLKTVKELLGITIMERSSFEKDPIAFLEAKGYKDVKKDLII | |
| | KLPKYSLFELENGRKRMLASAGELQKGNELALPSKYVNFLYLASHIEKLKGS | |
| | PEDEEKKQLYVEQHKHYLDEIIEQISEFSERVILADKNLDKVLSAYNNNRDK | |
| | SIEEQAENIIHLFTLTNLGAPAAFKYFGTTIDRKRYTSTKEVLDATLIHQSI | |
| | TGLYETRIDLSQLGGD | |
| CasEnd-71 | MDKKYSIGLDIGTNSVGWAVITDEYKVPSKKFKVLGNTDRHSIKKNLIGALL | 31 |
| | FDSGETAEATRLKRTARRRYTRRKNRIRYLQEIFAEEMAKVDDSFFHRLEES | |
| | FLVEEDKKNERHPIFGNIVDEVAYHEKYPTIYHLRKKLVDSDEKADLRLIYL | |
| | ALAHMIKFRGHFLIEGDLNSDNSDVQKLFIQLVETYNQLFEESPIEAEGVDA | |
| | KAILSEKLSKSRRLENLIAQLPGEKKNGLFGNLIALSLGLTPNFKSNFDLSE | |
| | DAKLQLSKDTYDDDLEELLGQIGDQYADLFLAAKNLSDAILLSGILRVSTES | |
| | TKAPLSASMIKRYDEHHQDLTLLKDLVRKQLPEKYKEIFFDQSKNGYAGYID | |
| | GGASQEEFYKYIKPILEKMDGTEELLDKLEREDLLRKQRTFDNGSIPHQIHL | |
| | GELHAILRRQEKYYPFLKENREKIEKILTFRIPYYVGPLARGNSRFAWMSRK | |
| | SDETITPWNFEEVVDKGASAEAFIERMTNFDKNLPDEKVLPKHSLLYEYFTV | |
| | YNELTKVKYVTEGMRKPEFLDGEQKKEIVDLLFKKNRKVTVKQLKEDYFKKF | |
| | ECFDIVEISGVEDRFNASLGTYHDLLKIIKDKEFLDNEENEEILEDIVLTLT | |
| | LFEDREMIKERLEKYADLFDKKVMKQLKRRRYTGWGRLSKKLINGIRDKQSG | |
| | KTILDFLKADGFANRNFMQLIHDDSLTFKDEIEKAQVTGDGESLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTEYDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIEVNEETGEIVWDKEKDFATVRKVLSMPQV | |
| | NIVKKTEVQTGGFSKESILPKRDSDKLIARKKDWDPKKYGGFDSPTVAYSVL | |
| | VVAKVEKGKTKKLKTVKELLGITIMERSAFEKNPVAFLEAKGYQEVKKDLII | |
| | KLPKYSLFELENGRKRMLASAGELQKGNELALPSKYVNFLYLASHYEKLKGS | |
| | PEDNEQKQEFVEQHKEYLDEIIDQISEFSKRVILADANLEKVKKAYEKHKDK | |
| | SIEEQAENIIHLFTLTALGAPAAFKYFGTTIDRKRYTSTKEVLNATLIHQSI | |
| | TGLYETRIDLSQLGGD | |
| CasEnd-72 | MKKKYSIGLDIGTNSVGWAVITDEYKVPSKKFKVLGNTDRQSIKKNLIGALL | 32 |
| | FDSGETAEATRLKRTARRRYTRRKNRIRYLQEIFAEEMSKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSENSDVDKLFIQLVQTYNQLFEENPINEEGVDA | |
| | KAILSAKLSKSRRLENLIAQFPGEKKNGLFGNLIALSLGLTPNFKSNEDLAE | |
| | DAKLQLSKDTYDDDLENLLAQIGDQYADLFLAAKNLSDAILLSDILRVNTEI | |
| | TKAPLSASMIKRYDEHHQDLTLLKALVRQQLPEKYKEIFFDESKNGYAGYID | |
| | GGASQEEFYKFIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMGKPEFLSGEQKKEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIEERLKKYANLFDDKVMKKLKRRRYTGWGRLSRKLINGIRDKQSG | |
| | KTILDFLKSDGFANRNFMQLIHDDSLTFKEAIKKAQVSGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGQELDINR | |
| | LSDYDVDHIVPQSFLKDDSIDNKVLTRSEEARGKSDNVPSEEVVKKMKPYWR | |
| | QLLKAKLITQRKFDNLTKAERGGLSELDKAGFIKRQLVETRQITKHVAQILD | |
| | SRMNTKRDENDKLIRDVKVITLKSKLVSDERKDFQFYKVREINNYHHAHDAY | |
| | LNAVVGTALIKKYPKLESEFVYGDYKVYDVRKMIAKSEQEIGKATAKYFFYS | |
| | NIMNFFKSEIKLANGEIRKRPLIETNEETGEIVWDKERDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELLGITIMERSSFEKDPVAFLEAKGYKDVKKDLII | |
| | KLPKYSLFELENGRKRMLASAGELQKGNELALPSKYVNFLYLASHYEKLKGS | |
| | PEDNEQKQLYVEQHKNYLDEIIDQISEFSERVILADKNLEKVLSAYNEFRDK | |
| | PIEEQAENIIHLFTLTNLGAPAAFKYFDKTIDRKRYTSTKEVLDATLIHQSI | |
| | TGLYETRIDLSQLGGD | |
| CasEnd-73 | MDKKYSIGLDIGTNSVGWAVITDEYKVPSKKFKVLGNTDRHSIKKNLIGALL | 33 |
| | FDSGETAEATRLKRTARRRYTRRKNRICYLQEIFSNEMAKVDDSFFHRLEES | |
| | FLVEEDKRHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEQIANLA | |
| | GSPAIKKGILQTIKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDDNDKLIRDVKIITLKSKLVSDFRKDFGLYKVREINNYHHAHDAY | |
| | LNAVVGTALIKKYPKLESEFVYGDYKVYDVRKMIAKSDKEIGKATAKYFFYS | |
| | NIMNFFKTDVTLANGEIRKRPLIEVNEETGEIVWDKEKDFATVRKVLSMPQV | |
| | NIVKKTEVQTGGFSKESILPKRNSDKLIARKKDWDTKKYGGFDSPTVAYSVL | |
| | VIAKVEKGKAKKLKTVKELVGITIMERSAFEKDPVAFLEAKGYQDIQKDLII | |
| | KLPKYSLFELENGRKRMLASAGELQKGNELALPQKYVTLLYLASKYEKLKGS | |
| | EEDNEKKQLYVEQHKEYFDEIMDQISEFAKRYILADANLEKIKSLYEKNFDA | |
| | SIEELAENFIHLLTFTNLGAPAAFKFFDTTIDRKRYTSTKEILNATLIHQSI | |
| | TGLYETRIDLSQLGGD | |
| CasEnd-74 | MDKKYSIGLDIGTNSVGWAVITDEYKVPSKKFKVLGNTDRQSIKKNLIGALL | 34 |
| | FDSGETAEATRLKRTARRRYTRRKNRIRYLQEIFSEEMSKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINESGVDA | |
| | KAILSARLSKSRRLENLIAQLPGEKKNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPTEKVLPKHSLLYEYFTV | |
| | YNELTKVKYVTEGMGKPEFLSGEQKQEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIEERLKKYANLFDDKVLKQLKRRRYTGWGRLSKKLINGIRDKQSG | |
| | KTILDFLKSDGFANRNFMQLIHDDSLTFKEEIKKAQVSGQGDSLHEQIANLA | |
| | GSPAIKKGILQTVKIVDELVKVMGRHKPENIVIEMARENQTTQKGQKNSRER | |
| | MKRLEEGLKELGSQILKEHPVENTQLQNEKLYLYYLQNGKDMYTGQELDIDR | |
| | LSDYDVDHIIPQSFIKDDSIDNKVLTSSDKARGKSDDVPSEEVVKKMKNYWR | |
| | QLLNAKLISQRKFDNLTKAERGGLSELDKAGFIKRQLVETRQITKHVAQILD | |
| | SRMNTEYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEAKGYKEVQKDLII | |
| | KLPKYSLFELENGRKRMLASAGELQKGNELALPSKYVTFLYLASHYEKLKGS | |
| | PEDNEKKQLFVEQHKHYFDEILDQISEFSKRYILADKNLDKVLSLYNKFRDK | |
| | SIREQAESIINLFTLTNLGAPAAFKFFDTTIDRKRYTSTKEILDSTLIHQSI | |
| | TGLYETRIDLSQLGGD | |
| CasEnd-75 | MDKKYSIGLDIGTNSVGWAVITDEYKVPSKKFKVLGNTDRHSIKKNLIGALL | 35 |
| | FDSGETAEATRLKRTARRRYTRRKNRICYLQEIFSNEMAKVDDSFFHRLEES | |
| | FLVEEDKKNERHPIFGNIVDEVAYHEKYPTIYHLRKKLADSTEKADLRLIYL | |
| | ALAHMIKFRGHFLIEGDLNSDNSDVQKLFIQLVQTYNQLFEENPLNESGVDA | |
| | KAILSARLSKSRRLENLIAQFPGEKKNGLFGNLIALSLGLTPNFKSNFDLAE | |
| | DAKLQLSKDTYDEDLENLLAQIGDQYADLFLAAKNLSDAILLSDILTVNDEI | |
| | TKAPLSASMVKRYDEHHQDLTLLKALVRQQLPEKYKEIFFDQSKNGYAGYID | |
| | GGASQEEFYKYIKPILEKIDGSEEFLAKINREDFLRKQRTFDNGSIPHQIHL | |
| | GELHAILRRQEDYYPFLKDNREKIEKILTFRIPYYVGPLARGNSRFAWMTRK | |
| | SEETITPWNFEEVVDKEASAQSFIERMTNFDKNLPNEKVLPKHSLLYEYFTV | |
| | YNELTKVKYVTEGMRKPEFLSGEQKQEIVDLLFKKNRKVTVKQLKEYLFKKI | |
| | ECFDSVEISGVEDRFNASLGTYHDLLKIIKDKDFLDNEENEKILEDIVLTLT | |
| | LFEDREMIEERLKKYAHLFDDKVLKQLKRRRYTGWGRLSRKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKNPVAFLEAKGYKEVQKDLII | |
| | KLPKYSLFELENGRKRLLASAGELQKGNELALPNKYVTLLYLASHYEKLKGS | |
| | PEDNEQKQLFVEQHKHYFDEIIEQISEFSKRVILADANLEKILSLYNKNRDA | |
| | SIEEQAENFIHLLTFTNLGAPAAFKFFDTTIDRKRYTSTKEILDATLIHQSI | |
| | TGLYETRIDLSQLGGD | |
| CasEnd-76 | MDKKYSIGLDIGTNSVGWAVITDEYKVPSKKFKVLGNTDRQSIKKNLIGALL | 36 |
| | FDSGETAEATRLKRTARRRYTRRKNRIRYLQEIFSNEMAKVDDSFFHRLEES | |
| | FLVEEDKKHERHPIFGNIVDEVAYHENYPTIYHLRKKLADSTEKADLRLIYL | |
| | ALAHMIKFRGHFLIEGDLNSDNSDVDKLFIQLVQTYNQLFEENPINESGVDA | |
| | KAILSAKLSKSRRLENLIAQLPGEKKNGLFGNLIALSLGLTPNFKSNFDLAE | |
| | DAKLQLSKDTYDDDLENLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDDKVLKQLKRRRYTGWGRLSKKLINGIRDKQSG | |
| | KTILDFLKSDGFANRNFMQLIHDDSLTFKEDIKKAQVSGQGDSLHEQIANLA | |
| | GSPAIKKGILQTVKIVDELVKVMGRHKPENIVIEMARENQTTQKGQKNSRER | |
| | MKRIEEGLKELGSQILKEHPVENTQLQNEKLYLYYLQNGKDMYTGDELDIDR | |
| | LSDYDVDHIIPQSFIKDDSIDNKVLTSSAEARGKSDNVPSEEVVKKMKSYWR | |
| | QLLKAKLISQRKFDNLTKAERGGLSELDKAGFIKRQLVETRQITKHVAQILD | |
| | SRMNTKRDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKTKKLKTVKELVGITIMERSSFEKDPVKFLEAKGYKEIQKDLII | |
| | KLPKYSLFELENGRKRLLASAGELQKGNELALPSKYVTFLYLASHYEKLKGS | |
| | PEDNEQKQLFVEQHKHYFDEILDQISEFSKRYILADKNLDKVLSLYNKFRDK | |
| | SISEQAENFINLFTLTNLGAPAAFKFFDTTIDRKRYTSTKEILDSTLIHQSI | |
| | TGLYETRIDLSQLGGD | |
| CasEnd-77 | MKKKYSIGLDIGTNSVGWAVITDEYKVPSKKFKVLGNTDRHSIKKNLIGALL | 37 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKELADSTEKADLRLIYL | |
| | ALAHMIKFRGHFLIEGDLNPDNSDVDKLFIQLVQTYNQLFEENPINESGVDA | |
| | KAILSAKLSKSRRLENLIAQLPGEKKNGLFGNLIALSLGLTPNFKSNEDLAE | |
| | DAKLQLSKDTYDDDLENLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPEEKVLPKHSLLYEYFTV | |
| | YNELTKVKYVTEGMKKPEFLSGEQKQEIVDLLFKKNRKVTVKQLKEDYFKKI | |
| | ECFDSVEISGVEDRFNASLGTYHDLLKIIKDKDELDNEENEDILEDIVLTLT | |
| | LFEDKEMIEERLKKYANLFDDKVLKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTSSKEARGKSDNVPSEEVVKKMKSYWR | |
| | QLLKAKLISQRKFDNLTKAERGGLSELDKAGFIKRQLVETRQITKHVAQILD | |
| | SRFNTERDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKTKKLKTVKELVGITIMERSSFEKDPIAFLEAKGYKEVQKDLII | |
| | KLPKYSLFELENGRKRMLASAGELQKGNELALPSKYVTFLYLASHYEKLKGS | |
| | PEDNEQKQLFVEQHKHYFDEILEQISEFSKRYILADKNLEKILSLYNNFEDK | |
| | SIREQAENIINLETLTNLGAPAAFKFFDTTIDRKRYTSTKEILDATLIHQSI | |
| | TGLYETRIDLSQLGGD | |
| CasEnd-78 | MKKKYSIGLDIGTNSVGWAVITDEYKVPSKKFKVLGNTDRHSIKKNLIGALL | 38 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINEEGVDA | |
| | KAILSAKLSKSRRLENLIAQLPGEKKNGLFGNLIALSLGLTPNFKSNEDLAE | |
| | DAKLQLSKDTYDEDLENLLAQIGDQYADLFLAAKNLSDAILLSDILTVNSEI | |
| | TKAPLSASMIKRYDEHHQDLTLLKKLVRQQLPEKYKEIFFDQSKNGYAGYID | |
| | GGASQEEFYKYIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYANLFDKKVLKQLKRRRYTGWGRLSKKLINGIRDKQSG | |
| | KTILDFLKSDGFANRNFMQLIHDDSLTFKEAIQKAQVSGQGDSLHEQIANLA | |
| | GSPAIKKGILQTVKIVDELVKVMGRHKPENIVIEMARENQTTQKGQKNSRER | |
| | MKRLEEGIKELGSQILKEHPVENTQLQNDKLYLYYLQNGKDMYTGKELDIDR | |
| | LSDYDVDHIIPQSFIKDDSIDNKVLTRSAKARGKSDNVPSEEVVKKMKSYWR | |
| | QLLNAKLISQRKFDNLTKAERGGLTELDKAGFIKRQLVETRQITKHVAQILD | |
| | SRFNTEYDENDKLIREVKIITLKSKLVSDFRKDFQFYKVREINDYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKDPVDFLEAKGYKEIQKDLII | |
| | KLPKYSLFELENGRKRMLASAGELQKGNELALPSKYVTFLYLASHYEKLKGK | |
| | PEDNEQKQLYVEQHRHYFDEIVEQISEFSKRYILADKNLEKVLSLYNNKRDK | |
| | SIREQAESIINLFTLTALGAPAAFKFFDTTIDRKRYTSTKEVLDATLIHQSI | |
| | TGLYETRIDLSQLGGD | |
| CasEnd-79 | MKKKYSIGLDIGTNSVGWAVITDEYKVPSKKFKVLGNTDRQSIKKNLIGALL | 39 |
| | FDSGETAEATRLKRTARRRYTRRKNRIRYLQEIFAEEMAKVDDSFFHRLEES | |
| | FLVEEDKKNERHPIFGNIVDEVAYHENYPTIYHLRKELVDSDEKADLRLIYL | |
| | ALAHMIKFRGHFLIEGDLNSENSDVQKLFIQLVQTYNQLFEENPLDEEGVDA | |
| | KAILSAKLSKSRRLENLIAQFPNEKKNGLFGNLIALSLGLTPNFKSNFGLSE | |
| | DAKLQLSKDTYDEDLENLLAQIGDQYADLFLAAKNLSDAILLSGILRVNSEI | |
| | TKAPLSASMIKRYDEHHQDLTLLKALVRQQLPEKYKEIFFDKSKNGYAGYID | |
| | GGASQEEFYKYIKPILEKMDGTEYLLAKLNREDLLRKQRTFDNGSIPHQIHL | |
| | GELHAILRRQEEYYPFLKENQEKIEKILTFRIPYYVGPLARGNSRFAWMTRK | |
| | SDETITPWNFEEVVDKGASAQAFIERMTNFDKNLPTEKVLPKHSLLYEYFTV | |
| | YNELTKVKYVTEGMGKPEFLSSEQKKEIVDLLFKKNRKVTVKQLKEFYFKEI | |
| | ECFDSVEISGVEDRFNASLGTYHDLLKIIKDKDFLDDEENEDILEDIVLTLT | |
| | LFEDREMIEKRLKKYANLFDKKVMKKLKRRRYTGWGRLSKKLINGIRDKQSG | |
| | KTILDFLKDDGFANRNFMQLIHDDSLTFKEEIKKAQVIGQGESLHEQIADLA | |
| | GSPAIKKGILQTVKVVDELVKVMGRHKPENIVIEMARENQTTQKGQRNSRER | |
| | MKRIEEGIKELGSDILKEHPVENTQLQNDKLYLYYLQNGRDMYTGDELDIDR | |
| | LSDYDVDHIVPQSFIKDDSIDNKVLTRSEKARGKSDDVPSEEVVKKMKSYWR | |
| | QLLKAKLITQRKFDNLTKAERGGLTELDKAGFIKRQLVETRQITKHVAQILD | |
| | SRMNTERDENDKLIRDVKVITLKSKLVSDFRKDFQFYKVREINDYHHAHDAY | |
| | LNAVVGTALIKKYPKLESEFVYGDYKVYDSRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-80 | MDKKYSIGLDIGTNSVGWAVITDEYKVPSKKFKVLGNTDRQSIKKNLLGALL | 40 |
| | FDSGETAEATRLKRTARRRYTRRKNRICYLQEIFAEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINESGVDA | |
| | KAILSAKLSKSRRLENLIAQFPGEKKNGLFGNLIALSLGLTPNFKSNEDLSE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDFLRKQRTFDNGSIPHQIHL | |
| | GELHAILRRQEAFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIILTLT | |
| | LFEDREMIEERLKKYAHLFDDKVMKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTSSKEARGKSDNVPSEEVVKKMKSYWR | |
| | QLLKAKLISQRKFDNLTKAERGGLSELDKAGFIKRQLVETRQITKHVAQILD | |
| | SRFNTERDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEIKLANGEIRKRPLIETNEETGEIVWDKEKDFATVRKVLSMPQV | |
| | NIVKKTEVQTGGFSKESILPKRESDKLIARKKDWDPKKYGGFDSPTVAYSVL | |
| | VVAKVEKGKSKKLKPVKELVGITIMERSSFEKNPVKFLEAKGYKDVQKDLII | |
| | KLPKYSLFELENGRKRMLASAGELQKGNELALPSKYVTFLYLASHYEKLKGS | |
| | PEDNEKKQLFVEQHKHYLDEIIEQISEFSKRYILADKNLDKILSLYNKNRDK | |
| | SIREQAENIINLFTLTALGAPAAFKFFDTTIDRKRYTSTKEILNSTLIHQSI | |
| | TGLYETRIDLSQLGGD | |
| CasEnd-81 | MDKKYSIGLDIGTNSVGWAVITDEYKVPSKKFKVLGNTDRHSIKKNLIGALL | 41 |
| | FDSGETAEATRLKRTARRRYTRRKNRIRYLQEIFANEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINESGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYAHLFDDKVMKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTSSDKARGKSDDVPSEEVVKKMKNYWR | |
| | QLLNAKLISQRKFDNLTKAERGGLSELDKAGFIKRQLVETRQITKHVAQILD | |
| | SRMNTERDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRDSDKLIARKKDWDPKKYGGFDSPTVAYSVL | |
| | VVAKVEKGKSKKLKTVKELVGITIMERSSFEKDPVDFLEAKGYKEVQKDLII | |
| | KLPKYSLFELENGRKRLLASAGELQKGNELALPSKYVTFLYLASHYEKLKGS | |
| | PEDNEQKQLFVEQHKHYLDEIVDQISEFSKRYILADKNLDKILSLYNKHRDK | |
| | PISEQAENIINLETLTNLGAPAAFKFFDTTIDRKRYTSTKEILDATLIHQSI | |
| | TGLYETRIDLSQLGGD | |
| CasEnd-82 | MDKKYSIGLDIGTNSVGWAVITDEYKVPSKKFKVLGNTDRHSIKKNLIGALL | 42 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKELADSTEKADLRLIYL | |
| | ALAHIIKFRGHFLIEGDLNSENSDVQKLFIQLVQTYNQLFEENPINEEGVDA | |
| | KAILSAKLSKSRRLENLIAQFPGEKKNGLFGNLIALSLGLTPNFKSNEDLAE | |
| | DAKLQLSKDTYDEDLDNLLAQIGDQYADLFLAAKNLSDAILLSDILTVNDEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQAFIERMTNFDKNLPTEKVLPKHSLLYEYFTV | |
| | YNELTKVKYVTEGMRKPEFLSGEQKKEIVDLLFKKNRKVTVKQLKEFLFKEI | |
| | ECFDSVEISGVEDRFNASLGTYHDLLKIIKDKDFLDNEENEDILEDIVLTLT | |
| | LFEDREMIEERLKKYADLFDKKVLKQLKRRRYTGWGRLSRKLINGIRDKQSG | |
| | KTILDFLKSDGFANRNFMQLIHDDSLTFKEEIKKAQVKGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKIEKGKSKKLKTVKELVGITIMERSSFEKDPIAFLEAKGYKNVQKDLII | |
| | KLPKYSLFELENGRKRLLASAGELQKGNELALPAKYVTLLYHASHYEKLKGS | |
| | PEDNEQKLLYVEQHRHYFDEILEQISEFSKRYILADKNLDKILSLYNKFRDL | |
| | SIEEQASSFINLFTLTALGAPAAFKFFDTTIDRKRYTSTKEILDATLIHQSI | |
| | TGLYETRIDLSQLGGD | |
| CasEnd-83 | MKKKYSIGLDIGTNSVGWAVITDEYKVPSKKFKVLGNTDRHSIKKNLLGALL | 43 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINEIGVDA | |
| | KAILSAKLSKSRRLENLIAQLPGEKKNGLFGNLIALSLGLTPNFKSNFDLAE | |
| | DAKLQLSKDTYDEDLENLLAQIGDQYADLFLAAKNLSDAILLSDILTVNDEI | |
| | TKAPLSASMIKRYDEHHQDLTLLKALVRQQLPEKYKEIFFDKSKNGYAGYID | |
| | GGASQEEFYKFIKPILEKMDGTEELLAKINREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDDKVLKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRLEEGIKELGSQILKEHPVENTQLQNDKLYLYYLQNGKDMYTGEELDIDR | |
| | LSDYDVDHIIPQSFIKDDSIDNKVLTSSKKARGKSDNVPSEEVVKKMKSYWR | |
| | QLLNAKLISQRKFDNLTKAERGGLSELDKAGFIKRQLVETRQITKHVAQILD | |
| | SRMNTEYDENDKLIREVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGEDSPTVAYSVL | |
| | VVAKVEKGKSKKLKTVKELVGITIMERSSFEKDPIDFLEAKGYKEVQKDLII | |
| | KLPKYSLFELENGRKRLLASAGELQKGNELALPSKYVTFLYLASHYEKLKGS | |
| | PEDNEKKQLYVEQHKHYFDEILEQISEFSERYILADKNLEKILSLYNKERDF | |
| | PIEEQAESIINLFTLTNLGAPAAFKFFGTTIDRKRYTSTKEILDATLIHQSI | |
| | TGLYETRIDLSQLGGD | |
| CasEnd-84 | MDKKYSIGLDIGTNSVGWAVITDEYKVPSKKFKVLGNTDRHSIKKNLLGALL | 44 |
| | FDSGETAEATRLKRTARRRYTRRKNRIRYLQEIFAEEMAKVDDSFFHRLEES | |
| | FLVEEDKRNERHPIFGNIVDEVAYHEKYPTIYHLRKKLADSDEKADLRLIYL | |
| | ALAHMIKFRGHFLIEGDLNSENSDVQKLFKQLVQTYNQLFEENPLNEEGVDA | |
| | KAILTARLSKSRRLENLIAQFPGEKKNGLFGNLIALSLGLTPNFKSNEDLAE | |
| | DAKLQLSKDTYDEDLENLLGQIGDQYADLFLAAKNLSDAILLSDILTVNDEI | |
| | TKAPLSASMVKRYDEHHQDLTLLKKLVRQQLPEKYKEIFFDQSKNGYAGYID | |
| | GGASQEEFYKYIKPILEKVDGSEEFLEKIEREDFLRKQRTFDNGSIPHQIHL | |
| | GELHAILRRQEEFYPFLKENREKIEKILTFRIPYYVGPLARGNSRFAWMTRK | |
| | SDETITPWNFEEVVDKEASAQKFIERMTNFDKNLPNEKVLPKHSLLYEKFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEKILEDIVLTLT | |
| | LFEDREMIEERLKKYADLFDDKVLKQLKRRRYTGWGRLSRKLINGIRDKQSG | |
| | KTILDFLKSDGFANRNFMQLIHDDSLTFKEEIEKAQVSGQGESLHELIANLA | |
| | GSPAIKKGILQTLKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQLYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGKDFATVKKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKNPVDFLEAKGYKNIKKDLII | |
| | KLPKYSLFELENGRKRLLASAGELQKGNELALPNKYVTLLYLASHYEKLKGS | |
| | PEDNEQKQLFVEQHKDYFDEIIEQISEFSKRVILADANLEKIKSLYEKNRDK | |
| | PIEEQAENFIHLFTFTNLGAPAAFKFFDTTIDRKRYTSTKEVLNATLIHQSI | |
| | TGLYETRIDLSQLGGD | |
| CasEnd-85 | MKKKYSIGLDIGTNSVGWAVITDEYKVPSKKFKVLGNTDRQSIKKNLLGALL | 45 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSENSDVDKLFIQLVQTYNQLFEENPINESGVDA | |
| | KAILSAKLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKQAIVDLLFKKNRKVTVKQLKEDYFKKI | |
| | ECFDSVEISGVEDRFNASLGTYHDLLKIIKDKEFLDNEENEDILEDIVLTLT | |
| | LFEDKEMIEERLKKYANLFDDKVMKQLKRRRYTGWGRLSKKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRLEEGIKELGSQILKEHPVENTQLQNDKLYLYYLQNGKDMYTGQELDIDR | |
| | LSDYDVDHIIPQSFIKDDSIDNKVLTSSKKARGKSDNVPSEEVVKKMKNYWR | |
| | QLLKAKLISQRKFDNLTKAERGGLSELDKAGFIKRQLVETRQITKHVAQILD | |
| | SRMNTKYDENDKLIRDVKIITLKSKLVSDFRKDFQFYKVREINDYHHAHDAY | |
| | LNAVVGTALIKKYPKLESEFVYGDYKVYDVRKMIAKSEQEIGKATAKRFFYS | |
| | NIMNFFKSEITLANGEIRKRPLIETNEETGEIVWDKGKDFATVRKVLSMPQV | |
| | NIVKKTEVQTGGFSKESILPKRNSDKLIARKKDWDPKKYGGFDSPTVAYSVL | |
| | VVAKVEKGKKKKLKTVKELVGITIMERSSFEKNPIAFLEAKGYKNVQKDLII | |
| | KLPKYSLFELENGRKRLLASAGELQKGNELALPSKYVTFLYLASHYEKLKGS | |
| | PEDNEQKQLFVEQHNHYLDEIVDQISEFSKRYILADANLDKILSLYNNFRDK | |
| | PINEQAENFINLFTLTALGAPAAFKFFNTTIDRKRYTSTKEILNATLIHQSI | |
| | TGLYETRIDLSQLGGD | |
| CasEnd-86 | MKKKYSIGLDIGTNSVGWAVITDEYKVPSKKFKVLGNTDRQSIKKNLLGALL | 46 |
| | FDSGETAEATRLKRTARRRYTRRKNRIRYLQEIFASEMSKIDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNTDVQKLFIQLVQTYNQLFEENPLDESGVDA | |
| | KAILTEKLSKSRRLENLIAQLPGEKKNGLFGNLIALSLGLTPNFKSNFELAE | |
| | DAKLQLSKDTYDEDLENLLAQIGDQYADLFVAAKNLSDAILLSDILTVNDEI | |
| | TKAPLSASMIKRYDEHHQDLTLLKALVRQQLPEKYKEIFFDESKNGYAGYID | |
| | GGASQEEFYKFIKPILEKLDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQAFIERMTNFDKNLPEEKVLPKHSLLYEYFTV | |
| | YNELTKVKYVTEGMGKPEFLSGEQKKEIVDLLFKKNRKVTVKQLKEDLFKEF | |
| | ECFDSVEISGVEDRFNASLGTYHDLLKIIKDKDFLDNEENEDILEDIILTLT | |
| | LFEDREMIEERLKKYADLFDDKVLKQLKRRRYTGWGRLSKKLINGIRDKQSG | |
| | KTILDFLKSDGFANRNFMQLIHDDSLTFKEEIKKAQVIGQTDSLHEQIANLA | |
| | GSPAIKKGILQTVKIVDELVKVMGRHKPENIVIEMARENQTTQKGQKNSRER | |
| | LKRIEEGIKELGSQILKEHPVENTQLQNEKLYLYYLQNGKDMYTGKELDIDR | |
| | LSDYDVDHIIPQSFIKDDSIDNKVLTRSEKARGKSDNVPSEEVVKKMKSYWQ | |
| | KLLNAKLISQRKFDNLTKAERGGLSELDKAGFIKRQLVETRQITKHVAQILD | |
| | SRFNTKRDENDKLIREVKIITLKSKLVSDFRKDFGFYKVREINDYHHAHDAY | |
| | LNAVVGTALIKKYPKLESEFVYGDYKVYDSRKMIAKSEQEIGKATAKRFFYS | |
| | NIMNFFKTEIKLADGEIIKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSSFEKNPVAFLEAKGYQNIQKDLII | |
| | KLPKYSLFELENGRKRLLASAGELQKGNELALPQKYVTFLYLASHYEKKKGS | |
| | PEDEEQKQLYVEQHKYYFDEIIDQISEFSKRYILADKNLDKVEELYNKNRDK | |
| | SIVELAESFINLFTFTALGAPAAFKFFDTTIDRKRYTSTKEILNSTLIHQSI | |
| | TGLYETRIDLSKLGGD | |
| CasEnd-87 | MDKKYSIGLDIGTNSVGWAVITDEYKVPSKKFKVLGNTDRHSIKKNLIGALL | 47 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKELADSTEKADLRLIYL | |
| | ALAHMIKFRGHFLIEGDLNSENTDVQKLFIQLVQTYNQLFEENPINEETVDA | |
| | KAILSAKLSKSRRLENLIAQFPGEKRNGLFGNLIALSLGLTPNFKSNEDLAE | |
| | DAKLQLSKDTYDEDLENLLGQIGDQYADLFLAAKNLSDAILLSDILTVNDEI | |
| | TKAPLSASMIKRYDEHHQDLTLLKALVRQQLPEKYKEIFFDESKNGYAGYID | |
| | GGASQEEFYKFIKPILEKMDGTEEFLAKINREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPNEKVLPKHSLLYEKFTV | |
| | YNELTKVKYVTEGMGKPEFLSGEQKQEIVDLLFKKNRKVTVKQLKEFYFKEI | |
| | ECFDSVEISGVEDRFNASLGTYHDLLKIIKDKDELDNEENEDILEDIVLTLT | |
| | LFEDREMIEKRLKKYANLFDKKVLKKLKRRRYTGWGRLSRKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRLEEGLKELGSQILKEHPVENTQLQNDKLYLYYLQNGKDMYTGEELDIDR | |
| | LSDYDVDHIIPQSFIKDDSIDNKVLTSSEKARGKSDNVPSEEVVKKMKSYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVAKVEKGKAKKLKTVKELVGITIMERSSFEKDPVAFLEAKGYKEIQKDLII | |
| | KLPKYSLFELENGRKRLLASAGELQKGNELALPQKYVTFLYLASHYEKLKGS | |
| | PEDNEKKQLYVEQHKHYFDEILDQISEFSKRYILADKNLEKILSLYNKNRDK | |
| | PISEQAESIINLFTLTALGAPAAFKFFGTTIDRKRYTSTKEILDATLIHQSI | |
| | TGLYETRIDLSQLGGD | |
| CasEnd-88 | MDKKYSIGLDIGTNSVGWAVITDEYKVPSKKFKVLGNTDRHSIKKNLIGALL | 48 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVQKLFIQLVQTYNQLFEENPINESGVDA | |
| | KAILSARLSKSRRLENLIAQFPGEKKNGLFGNLIALSLGLTPNFKSNFDLAE | |
| | DAKLQLSKDTYDEDLENLLAQIGDQYADLFLAAKNLSDAILLSDILTVNSEI | |
| | TKAPLSASMVKRYDEHHQDLTLLKQLVRQQLPEKYKEIFFDQSKNGYAGYID | |
| | GGASQEEFYKYIKPILEKMDGSEEFLDKIEREDFLRKQRTFDNGSIPHQIHL | |
| | GELHAILRRQEKYYPFLKDNREKIEKILTFRIPYYVGPLARGNSRFAWMTRK | |
| | SDETITPWNFEEVVDKGASAQSFIERMTNFDKNLPNEKVLPKHSLLYEKFTV | |
| | YNELTKVKYVTEGMRKPEFLSAEQKQEIVDLLFKKNRKVTVKQLKEDYFKKI | |
| | ECFDSVEISGVEDRFNASLGTYHDLLKIIKDKDFLDNEENEKILEDIVLTLT | |
| | LFEDREMIEERLKKYAHLFDDKVLKQLKRRRYTGWGRLSRKLINGIRDKQSG | |
| | KTILDFLKSDGFANRNFMQLIHDDSLTFKEEIEKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVAKVEKGKTKKLKTVKELVGITIMERSAFEKNPVDFLEAKGYKEIKKDLII | |
| | KLPKYSLFELENGRKRMLASAGELQKGNELALPQKYVTLLYLASHYEKLKGS | |
| | PEDNEQKQLFVEQHKHYFDEIIEQISEFSKRYILADANLEKILSLYEKHRDK | |
| | PIEELAENFIHLFTLTALGAPAAFKFFDTTIDRKRYTSTKEILDATLIHQSI | |
| | TGLYETRIDLSQLGGD | |
| CasEnd-89 | MKKKYSIGLDIGTNSVGWAVITDDYKVPSKKFKVLGNTDRKSIKKNLIGALL | 49 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSENSDVDKLFIQLVQTYNQLFEENPINESGVDA | |
| | KAILSAKLSKSRRLENLIAQFPGEKKNGLFGNLIALSLGLTPNFKKNFDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMINEDENLPEEKVLPKHSLLYEYFTV | |
| | YNELTKVKYVTEGMRKPEFLSGEQKKEIVDLLFKKNRKVTVKQLKEDYFKEI | |
| | ECFDSVEISGVEDRFNASLGTYHDLLKIIKDKDFLDNEENEDILEDIVLTLT | |
| | LFEDREMIEKRLKKYADLFDKKVMKQLKRRRYTGWGRLSRKLINGIRDKQSG | |
| | KTILDFLKSDGFANRNFMQLIHDDSLTFKEEIKKAQVKGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGDELDINR | |
| | LSQYDVDHIVPQSFLKDDSIDNKVLTRSDEARGKSDNVPSEEVVKKMKNYWR | |
| | QLLKAGLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKKYDVRKMIAKSEQEIGKATAKYFFYS | |
| | NIMNFFKTEITLANGEIRKRPLIETNEETGEIVWDKERDFATVRKVLSMPQV | |
| | NIVKKTEVQTGGFSKESILPKRNSDKLIARKKGWDPKKYGGFDSPTVAYSVL | |
| | VVAKVEKGKSKKLKTVKELLGITIMERSSFEKDPVAFLEAKGYKEVKKDLII | |
| | KLPKYSLFELENGRKRMLASAGELQKGNELALPSKYVNFLYLASHYEKLKGK | |
| | PEDNEQKQEYVEQHKHYLDEIIEQISEFSERVILADKNLSKVLSAYNEHRDK | |
| | PISEQAENIIHLFTLTNLGAPAAFKYFDTTIDRKRYTSTKEVLDATLIHQSI | |
| | TGLYETRIDLSQLGGD | |
| CasEnd-90 | MKKKYSIGLDIGTNSVGWAVITDEYKVPSKKFKVLGNTDRHSIKKNLIGALL | 50 |
| | FDSGETAEATRLKRTARRRYTRRKNRIRYLQEIFAEEMAKVDDSFFQRLEES | |
| | FLVEEDKKNERHPIFGNIVDEVAYHENYPTIYHLRKKLADSTEKADLRLIYL | |
| | ALAHIIKFRGHFLIEGDLNSENSDVQKLFEQLVQTYNQLFEEDPLNEEGIDA | |
| | EAILSAKLSKSRRLENLIAQIPGEKKNGLFGNLIALSLGLTPNFKANFDLAE | |
| | DAKLQLSKDTYDDDLENLLAQIGDQYADLFLAAKNLSDAILLSDILTVNGEI | |
| | TKAPLSASMIKRYDEHHQDLTLLKALVRKQLPEKYKEIFFDESKNGYAGYID | |
| | GGASQEEFYKYIKPILEKMDGAEEFLAKINREDFLRKQRTFDNGSIPHQIHL | |
| | GELHAILRRQEKFYPFLKENQEKIEKILTFRIPYYVGPLARGNSRFAWMTRK | |
| | SDETITPWNFEEVVDKEASAQAFIERMTNFDKNLPEEKVLPKHSLLYEYFTV | |
| | YNELTKVKYVTEGMGKPEFLSSEQKKEIVDLLFKKNRKVTVKQLKEDYFKKI | |
| | ECFDTVEISGVEDRFNASLGTYHDLLKIIKDKEFLDNEENEDILEDIVLTLT | |
| | LFEDKEMIEERLKKYADLFDKKVLKKLKRRRYTGWGRLSKKLINGIRDKQSG | |
| | KTILDFLKSDGFANRNFMQLIHDDSLTFKEEIKKAQVIGQKDSLHEQIANLA | |
| | GSPAIKKGILQTVKIVDELVKVMGRHAPENIVIEMARENQTTQKGQKNSRER | |
| | MKRLEEGVKELGSKILKEHPVENTQLQNDKLYLYYLQNGKDMYTGEELDIDR | |
| | LSDYDVDHIIPQSFIKDDSIDNKVLTSSAKARGKSDDVPSEEVVKKMKNYWR | |
| | KLLKAKLISQRKFDNLTKAERGGLSELDKAGFIKRQLVETRQITKHVAQILD | |
| | SRFNTERDENDKLIRDVKIITLKSKLVSDFRKDFQLYKVREINDYHHAHDAY | |
| | LNAVVGTALIKKYPKLESEFVYGDYKVYDSYKMIGKSDQEIGKATAKRFFYS | |
| | NIMNFFKTEITLANGEIRERPVIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSSFEKNPVAFLENKGYKDIQEDLII | |
| | KLPKYSLFELENGRKRLLASAGELQKGNELALPAKYVTFLYLASHYEKLKGS | |
| | PEDEEKKRLYVEQHEHYFDEIIDQIIEFSKRYILADKNLEKILSLYNENRDK | |
| | SISEQAESFINLFTLTALGAPAAFKFFDTTIDRKRYTSTKEILNATLIHQSI | |
| | TGLYETRIDLSKLGGD | |
| CasEnd-91 | MDKKYSIGLDIGTNSVGWAVITDEYKVPSKKFKVLGNTDRQSIKKNLIGALL | 51 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKELADSTEKADLRLIYL | |
| | ALAHMIKFRGHFLIEGDLNPDNSDVDKLFIQLVQTYNQLFEENPINESGVDA | |
| | KAILSAKLSKSRRLENLIAQLPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQAFIERMTNFDKNLPNEKVLPKHSLLYEYFTV | |
| | YNELTKVKYVTEGMKKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYAHLFDDKVMKQLKRRRYTGWGRLSRKLINGIRDKQSG | |
| | KTILDFLKSDGFANRNFMQLIHDDSLTFKEEIQKAQVKGQGDSLHEQIANLA | |
| | GSPAIKKGILQTVKIVDELVKVMGRHKPENIVIEMARENQTTQKGQKNSRER | |
| | MKRLEEGIKELGSQILKEHPVENTQLQNEKLYLYYLQNGKDMYTGQELDIDR | |
| | LSDYDVDHIIPQSFIKDDSIDNKVLTSSDKARGKSDNVPSEEVVKKMKSYWR | |
| | QLLNAKLISQRKFDNLTKAERGGLSELDKAGFIKRQLVETRQITKHVAQILD | |
| | SRMNTEYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKDPVAFLEAKGYKEIRKDLII | |
| | KLPKYSLFELENGRKRMLASAGELQKGNELALPSKYVTFLYLASHYEKLKGS | |
| | PEDNEQKQLFVEQHKHYLDEIIEQISEFSKRYILADKNLEKILSLYNNNEDK | |
| | SISEQAENIINLFTLTNLGAPAAFKFFDTTIDRKRYTSTKEILDATLIHQSI | |
| | TGLYETRIDLSQLGGD | |
| CasEnd-92 | MKKKYSIGLDIGTNSVGWAVITDDYKVPSKKFKVLGNTDRQSIKKNLIGALL | 52 |
| | FDSGETAEATRLKRTARRRYTRRKNRIRYLQEIFANEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSDNSDVDKLFIQLVQTYNQLFEENPINEEGVDA | |
| | KAILSAKLSKSRRLENLIAQLPGEKKNGLFGNLIALSLGLTPNFKSNEDLAE | |
| | DAKLQLSKDTYDDDLENLLAQIGDQYADLFLAAKNLSDAILLSDILRVNTEI | |
| | TKAPLSASMIKRYDEHHQDLTLLKALVRKQLPEKYKEIFFDESKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNFDENLPNEKVLPKHSLLYEYFTV | |
| | YNELTKVKYVTEGMGKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDKKVMKQLKRRRYTGWGRLSRKLINGIRDKQSG | |
| | KTILDFLKSDGFANRNFMQLIHDDSLTFKEEIKKAQVKGQGESLHEQIADLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGQELDINR | |
| | LSDYDVDHIVPQSFLKDDSIDNKVLTRSEKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKKYDVRKMIAKSEQEIGKATAKYFFYS | |
| | NIMNFFKSEIKLANGEIRKRPLIETNEETGEIVWDKERDFATVRKVLSMPQV | |
| | NIVKKTEVQTGGFSKESILPKRKSDKLIARKKGWDPKKYGGFDSPTVAYSVL | |
| | VVAKVEKGKAKKLKTVKELLGITIMERSSFEKDPVAFLEAKGYKEVKKDLII | |
| | KLPKYSLFELENGRKRMLASAGELQKGNELALPSKYVNFLYLASHYEKLKGK | |
| | PEDNEKKQLYVEQHKHYLDEIIEQISEFSERVILADKNLDKVLSAYNNIRDK | |
| | SIKEQAENIIHLFTLTNLGAPAAFKYFGTTIDRKRYTSTKEVLNATLIHQSI | |
| | TGLYETRIDLSQLGGD | |
| CasEnd-93 | MDKKYSIGLDIGTNSVGWAVITDEYKVPSKKFKVLGNTDRHSIKKNLIGALL | 53 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINEEGVDA | |
| | KAILSAKLSKSRRLENLIAQFPGEKRNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKFIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SDETITPWNFEEVVDKGASAQAFIERMTNFDKNLPTEKVLPKHSLLYEYFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDKKVLKKLKRRRYTGWGRLSKKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVAKVEKGKTKKLKTVKELVGITIMERSSFEKDPVAFLEAKGYKEVQEDLII | |
| | KLPKYSLFELENGRKRMLASAGELQKGNELALPNKYVTFLYLASHYEKLKGK | |
| | PEDEEQKQLYVEQHLDYFDEILDQISEFSKRYILADKNLEKILSLYNEFEDY | |
| | SISEQAESFINLFTFTALGAPAAFKFFGTTIDRKRYTSTKEILDSTLIHQSI | |
| | TGLYETRIDLSQLGGD | |
| CasEnd-94 | MDKKYSIGLDIGTNSVGWAVITDEYKVPSKKFKVLGNTDRHSIKKNLIGALL | 54 |
| | FDSGETAEATRLKRTARRRYTRRKNRICYLQEIFSNEMAKVDDSFFHRLEES | |
| | FLVEEDKKHERHPIFGNIVDEVAYHEEYPTIYHLRKKLADSTEKADLRLIYL | |
| | ALAHIIKFRGHFLIEGDLNSENSDVQKLFIQLVQTYNQLFEENPINEEGVDA | |
| | KAILSAKLSKSRRLENLIAQFPGEKKNGLFGNLIALSLGLTPNFKSNFGLSE | |
| | DAKLQLSKDTYDEDLENLLGQIGDQYADLFLAAKNLSDAILLSDILTVNDEI | |
| | TKAPLSASMIKRYDEHHQDLTLLKTFVRQQLPEKYKEIFFDPSKNGYAGYID | |
| | GGASQEEFYKYIKPILEKLDGTEELLAKINREDFLRKQRTFDNGSIPHQIHL | |
| | GELHAILRRQEEYYPFLKENREKIEKILTFRIPYYVGPLARGNSRFAWMTRK | |
| | SDETITPWNFEEVVDKGASAQAFIERMTNFDKNLPTEKVLPKHSLLYEYFTV | |
| | YNELTKVKYVTEGMGKPEFFSGNQKQEIVDGLFKKDRKVTVKQLKEFLFKEF | |
| | DEFRSVEISGVEDAFNASLGTYHDLLKIIKDKDFLDNEENEDILEDIVLTLT | |
| | LFEDKEMIEERLKKYADLFDDKVLKQLKRRRYTGWGRLSRKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-95 | MDKKYSIGLDIGTNSVGWAVITDEYKVPSKKFKVLGNTDRHSIKKNLIGALL | 55 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENSDVDKLFIQLVQTYNQLFEENPINEEGVDA | |
| | KAILSARLSKSRRLENLIAQFPGEKKNGLFGNLIALSLGLTPNFKSNEDLSE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKFIKPILEKMDGTEELLAKINREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKEASAQAFIERMTNFDKNLPNEKVLPKHSLLYEYFTV | |
| | YNELTKVKYVTEGMRKPEFLSAEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEKRLSKYANLFDKKVLKQLKRRRYTGWGRLSRKLINGIRDKQSG | |
| | KTILDFLKSDGFANRNFMQLIHDDSLTFKEAIKKAQVKGQGDSLHEHIANLA | |
| | GSPAIKKGILQTVKVVDELVKVMGRHKPENIVIEMARENQTTQKGQKNSRER | |
| | MKRIEEGLKELGSQILKEHPVENTQLQNEKLYLYYLQNGKDMYTGQELDIDR | |
| | LSDYDVDHIIPQSFIKDDSIDNKVLTSSEKARGKSDNVPSEEVVKKMKNYWR | |
| | QLLKAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKDPIAFLEAKGYKEIQKDLII | |
| | KLPKYSLFELENGRKRLLASAGELQKGNELALPSKYVTFLYLASHYEKLKGS | |
| | PEDNEKKQLYVEQHLHYFDEILDQISEFSERYILADKNLEKILELYNKNEDY | |
| | SISEQAESIINLFTLTALGAPAAFKFFGTTIDRKRYTSTKEILDSTLIHQSI | |
| | TGLYETRIDLSQLGGD | |
| CasEnd-96 | MKKKYSIGLDIGTNSVGWAVITDEYKVPSKKFKVLGNTDRHSIKKNLLGALL | 56 |
| | FDSGETAEATRLKRTARRRYTRRKNRIRYLQEIFASEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINESGVDA | |
| | KAILSARLSKSRRLENLIAQLPGEKRNGLFGNLIALSLGLTPNFKSNEDLAE | |
| | DAKLQLSKDTYDEDLDNLLGQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPTEKVLPKHSLLYEYFTV | |
| | YNELTKVKYVTEGMRKPEFLSGEQKKAIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDKKVLKQLKRRRYTGWGRLSRKLINGIRDKQSG | |
| | KTILDFLKSDGFANRNFMQLIHDDSLTFKEIIKKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTRSDKARGKSDNVPSEEVVKKMKNYWR | |
| | QLLNAKLISQRKFDNLTKAERGGLSELDKAGFIKRQLVETRQITKHVAQILD | |
| | SRMNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKDPVAFLEAKGYKEVQKDLII | |
| | KLPKYSLFELENGRKRLLASAGELQKGNELALPSKYVTFLYLASHYEKLKGS | |
| | PEDNEKKQLFVEQHRHYLDEIIDQISEFSKRYILADKNLDKLLSLYNNHRDK | |
| | SISEQAENFINLFTLTNLGAPAAFKFFDTTIDRKRYTSTKEILDSTLIHQSI | |
| | TGLYETRIDLSQLGGD | |
| CasEnd-97 | MKKKYSIGLDIGTNSVGWAVITDEYKVPSKKFKVLGNTDRQSIKKNLLGALL | 57 |
| | FDSGETAEATRLKRTARRRYTRRKNRIRYLQEIFAEEMNKVDDSFFHRLEES | |
| | FLVEEDKKGERHPIFGNIVDEVAYHEKYPTIYHLRKKLADSTEKADLRLIYL | |
| | ALAHMIKFRGHFLIEGDLNPDNSDVDKLFIQLVQTYNQLFEESPINEEGVDA | |
| | KAILSAKLSKSRRLENLIAQIPGEKRNGLFGNLIALSLGLTPNFKSNEDLAE | |
| | DAKLQLSKDTYDEDLDNLLAQIGDQYADLFLAAKNLSDAILLSGILTVNDEN | |
| | TKAPLSASMIKRYDEHHQDLTLLKQLVRQQLPEKYKEIFFDDSKNGYAGYID | |
| | GGASQEDFYKYIKPILEKLDGAEELLAKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPSEKVLPKHSLLYEYFTV | |
| | YNELTKVKYITEGMGKPEFLSAEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLEKYAHLFDDKVLKQLKRRRYTGWGRLSKKLINGIRDKQSG | |
| | KTILDFLKSDGFANRNFMQLIHDDSLTFKEEIQKAQVIGQGDSLHEQIADLA | |
| | GSPAIKKGILQSIKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKRDENDKLIREVKIITLKSKLVSDFRKDFGFYKVREINDYHHAHDAY | |
| | LNAVVGLALIKKYPKLESEFVYGDYKVYDVRKMIAKSEQEIGKATAKYFFYS | |
| | NIMNFFKSEITLANGEIRKRPQIETNEETGEIVWNKVKDFATVRKVLSMPQV | |
| | NIVKKTEVQTGGFSKESILPKRESDKLIARKKDWDPKKYGGFDSPTVAYSVL | |
| | VVAKVEKGKAKKLKTVKELVGITIMERSSFEKDPVAFLEAKGYKNIQKDLII | |
| | KLPKYSLFELENGRKRLLASAGELQKGNELALPAKYVTLLYLASNYEKLKGS | |
| | PEDNEQKLEYVEQHKEYFKEILDQIIEFSSRYILADKNLDKVKSLYAEHRDK | |
| | DITELAENFIHLFTLTSLGAPAAFKFFGTTIDRKRYTSTSEILNATLIHQSI | |
| | TGLYETRIDLSQLGGD | |
| CasEnd-98 | MDKKYSIGLDIGTNSVGWAVITDEYKVPSKKFKVLGNTDRKSIKKNLIGALL | 58 |
| | FDSGETAEATRLKRTARRRYTRRKNRICYLQEIFSEEMAKVDDSFFHRLEES | |
| | FLVEEDKRHERHPIFGNIVEEVAYHEKYPTIYHLRKKLVDSDDKADLRLIYL | |
| | ALAHMIKFRGHFLIEGDLNPDNSDVDKLFIQLVQTYNQLFEENPIDAEKVDA | |
| | EAILTERLSKSRRLENLIAELPGEKKNGLFGNLIALSLGLTPNFKSNEDLSE | |
| | DAKLQLSKDTYDEDLENLLGQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | QELHAILRRQEDFYPFLKDNREKIEKILTFRIPYYVGPLARGNSRFAWMTRK | |
| | SDETITPWNFDEVVDKGASAQSFIERMTNFDKNLPNEKVLPKHSLLYEYFTV | |
| | YNELTKVKYVTEGMRKPAFLDGEQKKEIVDLLFKTNRKVTVKQLKEDYFKKI | |
| | DCFDIVEISGVEDRFNASLGTYHDLLKIIKDKDFLDNEENEDILEDIVLTLT | |
| | LFEDREMIEKRLKTYANLFDKKVMKQLKRRRYTGWGRLSRKLINGIRDKQSG | |
| | KTILDFLKSDGFTNRNFMQLIHDDSLTFKEDIQKAQVSGQGDSLHEQIANLA | |
| | GSPAIKKGILQTIKVVDELVKVMGRHKPENIVIEMARENQTTQKGQKNSRER | |
| | MKRIEEGIKELGSKILKEHPVENTQLQNEKLYLYYLQNGRDMYTDQELDINR | |
| | LSDYDVDHIVPQSFLKDDSIDNKVLTSSEKARGKSDDVPSEEVVKKMKPYWR | |
| | QLLNAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKVYDVRKMVAKSEQEIGKATAKYFFYS | |
| | NIMNFFKTEVKLADGEIRKRPLIEVNEETGEIVWDKGRDFATVRKVLSMPQV | |
| | NIVKKTEVQTGGFSKESILPKRNSDKLIARKKDWDPSKYGGFDSPTVAYSVL | |
| | VIAKIEKGKAKKLKSVKELLGITIMERSSFEKNPVDFLEAKGYQNIQKELII | |
| | KLPKYSLFELENGRKRLLASAGELQKGNELALPNKYVTFLYHAQHYEKLKGK | |
| | PEDEEYKQLFVEQHRHYFDEILEQIIEFSERYILADANLEKIKNLYDQHFDA | |
| | SLREQASNIINLETFTNLGAPAAFKYLDTDIDRKRYTSTKEILNATLIHQSI | |
| | TGLYETRIDLSQLGGD | |
| CasEnd-99 | MKKKYSIGLDIGTNSVGWAVITDEYKVPSKKFKVLGNTDRQSIKKNLIGALL | 59 |
| | FDSGETAEATRLKRTARRRYTRRKNRIRYLQEIFAEEMSKVDDSFFHRLEES | |
| | FLVEEDKRGERHPIFGNIVDEVAYHEKYPTIYHLRKELVDSTEKADLRLIYL | |
| | ALAHMIKFRGHFLIEGDLNSENTDVQKLFKQLVQAYNQLFEESPLNEETVDA | |
| | KAILTEKLSKSRRLENLIAQLPGEKKNGLFGNLIALSLGLTPNFKTNFGLAE | |
| | DAKLQLSKDTYDEDLENLLGQIGDQYADLFLAAKNLSDAILLSDILRVNDEI | |
| | TKAPLSASMIKRYDEHHQDLTLLKTLVREQLPEKYKEIFFDKSKNGYAGYID | |
| | GGASQEEFYKYIKPILEKMDGTEYLLAKLEREDLLRKQRTFDNGSIPHQIHL | |
| | GELHAILRRQEEYYPFLKENQEKIEKILTFRIPYYVGPLARGNSRFAWMSRK | |
| | SDETITPWNFEEVVDKGASAQAFIERMTNFDKNLPNEKVLPKHSLLYEKFTV | |
| | YNELTKVKYVTEGMGKPEFLDAEQKKEIVDGLFKKYRKVTVKKLKEFYFKEF | |
| | DEFRIVDISGVEDRFNASLGTYHDLLKIIKDKDFLDNDENEDILEDIVLTLT | |
| | LFEDREMIEERLKKYADLFDDKVMKQLKRRRYTGWGRLSKKLINGIRDKQSG | |
| | KTILDFLKSDGFANRNFMQLIHDDSLTFKEEIKKAQVKGQKDSLHEQIADLA | |
| | GSPAIKKGILQTVKVVDELVKVMGRHKPENIVIEMARENQTTQKGQKNSRER | |
| | YKRIEEGIKELGSQILKEHPVENTQLQNDKLYLYYLQNGRDMYTGEELDIDR | |
| | LSDYDVDHIVPQSFIKDDSIDNKVLTRSAEARGKSDNVPSEEVVKKMKSYWR | |
| | QLLKAKLITQRKFDNLTKAERGGLTELDKAGFIKRQLVETRQITKHVAQILD | |
| | SRMNTERDENDKLIRDVKVITLKSKLVSDFRKDFGFYKVREINDYHHAHDAY | |
| | LNAVVGTALIKKYPKLESEFVYGDYKVYDSYKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-100 | MKKKYSIGLDIGTNSVGWAVITDEYKVPSKKFKVLGNTDRQSIKKNLIGALL | 60 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSENSDVDKLFIQLVQTYNQLFEENPINESGVDA | |
| | KAILSAKLSKSRRLENLIAQLPNEKKNGLFGNLIALSLGLTPNFKSNFDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEDFYKFIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMGKPEFLSGEQKKEIVDLLFKKNRKVTVKQLKEFYFKKI | |
| | ECFDSVEISGVEDRFNASLGTYHDLLKIIKDKDFLDNEENEDILEDIVLTLT | |
| | LFEDREMIEERLKKYANLFDKKVMKQLKRRRYTGWGRLSRKLINGIRDKQSG | |
| | KTILDFLKSDGFANRNFMQLIHDDSLTFKEEIKKAQVSGQGESLHEQIANLA | |
| | GSPAIKKGILQTVKVVDELVKVMGRHKPENIVIEMARENQTTQKGQKNSRER | |
| | MKRIEEGIKELGSQILKEHPVENTQLQNDKLYLYYLQNGRDMYTGQELDINR | |
| | LSDYDVDHIVPQSFLKDDSIDNKVLTRSAEARGKSDNVPSEEVVKKMKNYWR | |
| | QLLKAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKVYDVRKMIGKSEQEIGKATAKYFFYS | |
| | NIMNFFKTEITLANGEIRKRPLIETNEETGEIVWDKERDFATVRKVLSMPQV | |
| | NIVKKTEVQTGGFSKESILPKRESDKLIARKKDWDPKKYGGFDSPTVAYSVL | |
| | VVAKVEKGKSKKLKTVKELLGITIMERSSFEKDPVAFLEAKGYKEVKKDLII | |
| | KLPKYSLFELENGRKRMLASAGELQKGNELALPSKYVNFLYLASHYEKLKGS | |
| | PEDNEKKQEYVEQHKHYLDEIIDQISEFSERVILADKNLEKVLSAYNEHRNK | |
| | SIEEQAENIIHLFTLTNLGAPAAFKYFGTTIDRKRYTSTKEVLNATLIHQSI | |
| | TGLYETRIDLSQLGGD | |
| CasEnd-101 | MDKKYSIGLDIGTNSVGWAVITDEYKVPSKKFKVLGNTDRHSIKKNLIGALL | 61 |
| | FDSGETAEATRLKRTARRRYTRRKNRIRYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINEEGVDA | |
| | KAILSARLSKSRRLENLIAQFPGEKKNGLFGNLIALSLGLTPNFKSNEDLAE | |
| | DAKLQLSKDTYDEDLENLLGQIGDQYADLFLAAKNLSDAILLSDILTVNDEI | |
| | TKAPLSASMVKRYDEHHQDLTLLKKLVRQQLPEKYKEIFFDQSKNGYAGYID | |
| | GGASQEEFYKYIKPILEKMDGSEEFLEKINREDFLRKQRTFDNGSIPHQIHL | |
| | GELHAILRRQEKYYPFLKDNREKIEKILTFRIPYYVGPLARGNSRFAWMTRK | |
| | SEETITPWNFEEVVDKEASAQKFIERMTNFDKNLPNEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIEERLKKYAHLFDDKVLKQLKRRRYTGWGRLSRKLINGIRDKQSG | |
| | KTILDFLKSDGFANRNFMQLIHDDSLTFKEEIKKAQVSGQGDSLHEQIANLA | |
| | GSPAIKKGILQTVKIVDELVKVMGRHKPENIVIEMARENQTTQKGQKNSRER | |
| | MKRLEEGIKELGSKILKEHPVENTQLQNEKLYLYYLQNGKDMYTGEELDIDR | |
| | LSDYDVDHIVPQSFLKDDSIDNKVLVSSEKARGKSDNVPSEEVVKKMKNYWR | |
| | QLLEAKLITQRKFDNLTKAERGGLTELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKNPIDFLEAKGYKEVKKDLII | |
| | KLPKYSLFELENGRKRMLASAGELQKGNELALPSKYVTLLYLASHYEKLKGS | |
| | PEDNEQKQLFVEQHKHYFDEIIEQISEFSKRVILADANLEKILSLYNKNRDK | |
| | SIEEQAENFINLFTFTNLGAPAAFKFFDTTIDRKRYTSTKEILDATLIHQSI | |
| | TGLYETRIDLSQLGGD | |
| CasEnd-102 | MDKKYSIGLDIGTNSVGWAVITDEYKVPSKKFKVLGNTDRHSIKKNLIGALL | 62 |
| | FDSGETAEATRLKRTARRRYTRRKNRICYLQEIFSNEMAKVDDSFFHRLEES | |
| | FLVEEDKRHERHPIFGNIVDEVAYHEKYPTIYHLRKKLADSDEKADLRLIYL | |
| | ALAHMIKFRGHFLIEGDLNSENSDVQKLFKQLVQTYNQLFEESPLNEEGVDA | |
| | KAILTEKLSKSRRLENLIAQFPGEKKNGLFGNLIALSLGLTPNFKSNEDLSE | |
| | DAKLQFSKDTYDEDLENLLGQIGDQYADLFLAAKNLYDAILLSGILTVNDEI | |
| | TKAPLSASMVKRYDEHHQDLTLLKQFVRQQLPEKYKEIFFDQSKNGYAGYID | |
| | GGASQEEFYKYLKKILEKIDGSEEFLEKINREDFLRKQRTFDNGSIPHQIHL | |
| | QELHAILRRQEKYYPFLKENQEKIEKILTFRIPYYVGPLARGNSRFAWMSRK | |
| | SDETITPWNFEDVVDKEASAEKFIERMTNFDKNLPEEKVLPKHSLLYEKFTV | |
| | YNELTKVKYVTEGMRKPVFFSAEQKQEIVDLLFKKNRKVTKKQLKEYLFKEF | |
| | ECFDSVEISGVEDRFNASLGTYHDLLKIIKDKDELDNPENEKILEDIVLTLT | |
| | LFEDREMIKKRLEKYADLFDKKQLKKLKRRRYTGWGRLSAKLINGIRDKQSG | |
| | KTILDYLIEDGFANRNFMQLIHDDSLTFKEEIEKAQVIGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-103 | MDKKYSIGLDIGTNSVGWAVITDEYKVPSKKFKVLGNTDRHSIKKNLIGALL | 63 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVDKLFIQLVQTYNQLFEENPINESGVDA | |
| | KAILSAKLSKSRRLENLIAQLPGEKKNGLFGNLIALSLGLTPNFKSNEDLSE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKFIKPILEKMDGTEELLEKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKGASAQAFIERMTNFDKNLPDEKVLPKHSLLYEYFTV | |
| | YNELTKVKYVTEGMRKPEFLSGEQKKEIVDLLFKKNRKVTVKQLKEDLFKEI | |
| | ECFDSVEISGVEDRFNASLGTYHDLLKIIKDKDFLDNEENEDILEDIVLTLT | |
| | LFEDREMIEERLKKYANLFDKKVLKQLKRRRYTGWGRLSRKLINGIRDKQSG | |
| | KTILDFLKSDGFANRNFMQLIHDDSLTFKEEIQKAQVIGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSSFEKDPVSFLEAKGYKEVQKDLII | |
| | KLPKYSLFELENGRKRLLASAGELQKGNELALPSKYVTFLYLAKHYEKLKGS | |
| | PEDNEQKQLYVEQHKNYFDEILDQISEFSKRYILADANLEKILSLYSNNEDK | |
| | PISEQASSFINLFTLTNLGAPAAFKFFDTTIDRKRYTSTKEILNATLIHQSI | |
| | TGLYETRIDLSQLGGD | |
| CasEnd-104 | MDKKYSIGLDIGTNSVGWAVITDEYKVPSKKFKVLGNTDRHSIKKNLIGALL | 64 |
| | FDSGETAEATRLKRTARRRYTRRKNRICYLQEIFSNEMAKVDDSFFHRLEES | |
| | FLVEEDKKNERHPIFGNIVDEKAYHEKYPTIYHLRKKLADSDEKADLRLIYL | |
| | ALAHMIKFRGHFLIEGDLNSDNSDVQKLFKQLVQTYNQLFEENPLNEETVDA | |
| | KAILTEKLSKSRRLENLIAQFPGEKKNGLFGNLIALSLGLTPNFKSNFDLAE | |
| | DAKLQLSKDTYDEDLENLLGQIGDEYADLFLAAKNLYDAILLSDILTVNDEI | |
| | TKAPLSASMVKRYEEHQKDLKLLKKFVRQQLPEKYKEIFSDKSKNGYAGYID | |
| | GGTSQEEFYKYLKKILEKMDGSEEFLEKIEREDFLRKQRTFDNGSIPHQIHL | |
| | QELHAILRRQEEYYPFLKENREKIEKILTFRIPYYVGPLARGNSRFAWMTRK | |
| | SDEKITPWNFDEVVDKEASAQKFIERMINNDLYLPEEKVLPKHSLLYEKFTV | |
| | YNELTKVKYVTEGMRKPEFFSAEQKQEIFDLLFKKNRKVTKKKLKEYLFKKF | |
| | ECFDIVEISGLEDRFNASLGTYHDLLKIIKDKDELDNEENEEILEDIVLTLT | |
| | LFEDREMIKKRLKKYADLFDDKVLKKLKRRHYTGWGRLSKKLINGIRDKQSG | |
| | KTILDYLISDGFANRNFMQLIHDDSLTFKEEIEKAQVSGDGDSLHEHIANLA | |
| | GSPAIKKGILQTVKVVDELVKVMGRHKPENIVIEMARENQTTQKGQRNSRQR | |
| | LKRLEEGIKELGSDILKEHPVENTQLQNDRLYLYYLQNGKDMYTGEELDIDR | |
| | LSDYDIDHIIPQSFIKDDSIDNKVLVSSKKARGKSDNVPSEEVVKKMKNYWR | |
| | KLLDAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLINLGAPAAFKYFDTTIDRKRYTSTKEVLDATLIHQSI | |
| | TGLYETRIDLSQLGGD | |
| CasEnd-105 | MDKKYSIGLDIGTNSVGWAVITDEYKVPSKKFKVLGNTDRHSIKKNLIGALL | 65 |
| | FDSGETAEATRLKRTARRRYTRRKNRICYLQEIFSNEMAKVDDSFFHRLEES | |
| | FLVEEDKKHERHPIFGNIVEEVAYHEKYPTIYHLRKKLADSDEKADLRLIYL | |
| | ALAHIIKFRGHFLIEGDLNSDNSDVQKLFKQLVQTYNQLFEESPLQEEGVDA | |
| | KAILSEKLSKSRRLENLIAQFPGEKKNGLFGNLIALSLGLTPNFKSNFDLSE | |
| | DAKLQLSKDTYDEDLENLLGQIGDQYADLFLAAKNLYDAILLSGILTVNDEI | |
| | TKAPLSASMVKRYDEHHQDLTLLKQLVREQLPEKYKEIFFDQSKNGYAGYID | |
| | GGASQEEFYKYIKKILEKIDGSEEFLDKINREDFLRKQRTFDNGSIPHQIHL | |
| | QELHAILRRQEKYYPFLKENREKIEKILTFRIPYYVGPLARGNSRFAWMSRK | |
| | SDETITPWNFEEVVDKEASAQAFIERMTNFDKNLPEEKVLPKHSLLYEKFTV | |
| | YNELTKVKYVTEGMRKPEFLSAEQKQEIFDLLFKKNRKVTKKQLKEYLFKNF | |
| | ECFDIVEISGVEDRFNASLGTYHDLLKIIKDKDFLDNEENEEILEDIVLTLT | |
| | LFEDREMIKERLEKYADLFDKKQLKKLKRRRYTGWGRLSKKLINGIRDKQSG | |
| | KTILDFLISDGFANRNFMQLIHDDSLTFKEEIEKAQVSGKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-106 | MKKKYSIGLDIGTNSVGWAVITDEYKVPSKKFKVLGNTDRSSIKKNLIGALL | 66 |
| | FDSGETAEATRLKRTARRRYTRRKNRIRYLQEIFAEEMSKVDDSFFHRLEES | |
| | FLVEEDKRDERHPIFGNIVDEVAYHEKYPTIYHLRKELVDSTEKADLRLIYL | |
| | ALAHMIKFRGHFLIEGDLNSENTDVQKLFIQLVQTYNQTFEENPLSEETVDA | |
| | EAILTDKLSKSRRLENLIAQFPNEKRNGLFGNLIALSLGLTPNFKSNENLAE | |
| | DAKLQFSKDTYDEDLENLLAQIGDQYADLFLAAKNLYDAILLSGILRVNDES | |
| | TKAPLSASMIKRYDEHHQDLTLLKQLVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEEYYPFLKENQEKIEKILTFRIPYYVGPLARGNSRFAWMTRK | |
| | SDETITPWNFEEVVDKGASAEAFIERMTNFDKNLPEEKVLPKHSLLYEKFTV | |
| | YNELTKVKYVTEGMGKPQFLDAEQKKEIVDLLFKKDRKVTVKQLKEFYFKEI | |
| | DCFRIVDISGVEDRFNASLGTYHDLLKIIKDKAFLDNEENEKILEDIVLTLT | |
| | LFEDREMIKKRLEKYANLFDKKVMKKLKRRRYTGWGRLSRKLINGIRDKQSG | |
| | KTILDFLISDGFANRNFMQLIHDDSLTFKEEIKKAQVEGQGESLHEQIADLA | |
| | GSPAIKKGILQTVKVVDELVKVMGRHKPENIVIEMARENQTTQKGQKNSRER | |
| | LKRIEEGIKELGSQILKEHPVENTQLQNEKLYLYYLQNGRDMYTGEELDIDR | |
| | LSDYDVDHIVPQSFIKDDSIDNKVLTRSKEARGKSDDVPSEEVVRKMKSYWR | |
| | QLLKAKLITQRKFDNLTKAERGGLTELDKAGFIKRQLVETRQITKHVAQILD | |
| | SRMNTERDENDKLIRDVKVITLKSKLVSDFRKDFEFYKVREINDYHHAHDAY | |
| | LNAVVGTALIKKYPKLESEFVYGDYPVYNSYKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-107 | MKKKYSIGLDIGTNSVGWAVITDEYKVPSKKFKVLGNTDRHSIKKNLIGALL | 67 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINEEGVDA | |
| | KAILSAKLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKKAIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYANLFDDKVLKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGKDMYTGQELDIDR | |
| | LSDYDVDHIIPQSFIKDDSIDNKVLTSSDKARGKSDNVPSEEVVKKMKNYWR | |
| | QLLNAKLISQRKFDNLTKAERGGLSELDKAGFIKRQLVETRQITKHVAQILD | |
| | SRFNTEYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKTKKLKTVKELVGITIMERSSFEKDPVDFLEAKGYKEIQKDLII | |
| | KLPKYSLFELENGRKRMLASAGELQKGNELALPSKYVTFLYLASHYEKLKGS | |
| | PEDEEQKQLYVEQHRHYFDEIVEQISEFSERYILADKNLEKILSLYNEFEDK | |
| | PIEEQAENFINLFTLTALGAPAAFKFFDTTIDRKRYTSTKEILDATLIHQSI | |
| | TGLYETRIDLSQLGGD | |
| CasEnd-108 | MDKKYSIGLDIGTNSVGWAVITDEYKVPSKKFKVLGNTDRHSIKKNLIGALL | 68 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKELADSTEKADLRLIYL | |
| | ALAHMIKFRGHFLIEGDLNSENSDVQKLFIQLVQTYNQLFEENPINEEGVDA | |
| | KAILTAKLSKSRRLENLIAQFPGEKKNGLFGNLIALSLGLTPNFKSNFVLSE | |
| | DAKLQLSKDTYDEDLENLLGQIGDQYADLFLAAKNLSDAILLSDILTVNSEI | |
| | TKAPLSASMIKRYDEHHQDLTLLKNLVRQQLPEKYKEIFFDKSKNGYAGYID | |
| | GGASQEEFYKYIKPILEKMDGTEEFLAKIEREDFLRKQRTFDNGSIPHQIHL | |
| | KELHAILRRQEEYYPFLKENREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMGKPQFLSAEQKQEIVDLLFKKERKVTKKQLKDFLFKEI | |
| | EEFDSVEISGVEDAFNASLGTYHDLLKIIKDKDFLDNEENEDILEDIILTLT | |
| | LFEDREMIEKRLKKYANLFDKKVLKQLKRRRYTGWGRLSKKLINGIRDKQSG | |
| | KTILDFLKSDGFANRNFMQLIHDDSLTFKEEIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-109 | MDKKYSIGLDIGTNSVGWAVITDEYKVPSKKFKVLGNTDRHSIKKNLIGALL | 69 |
| | FDSGETAEATRLKRTARRRYTRRKNRICYLQEIFSNEMAKVDDSFFHRLEES | |
| | FLVEEDKRGERHPIFGNIADEKAYHEKYPTIYHLRKKLADSTEKADLRLIYL | |
| | ALAHMIKFRGHFLIEGDLNSDNSDVQKLFDELVQTYNQLFEESPLNEETVDA | |
| | EAILTEKLSKSRRLENLIAQFPGEKKNGLFGNLIALSLGLTPNFKSNFDLSE | |
| | DAKLQFSKDTYDEDLEELLGQIGDEYADLFLAAKNLYDAILLSGILTVNDES | |
| | TKAPLSASMVKRYEEHQQDLKLLKKFIRQQLPEKYKEIFSDKSKNGYAGYID | |
| | GKTSQEEFYKYLKKILEKLDGSEEFLDKIEREDFLRKQRTFDNGSIPHQIHL | |
| | NELHAILRRQEEYYPFLKENQEKIEKILTFRIPYYVGPLARGNSRFAWMERK | |
| | SDEKITPWNFDEVVDKEASAEKFIERMTNNDLYLPEEKVLPKHSLLYEKFTV | |
| | YNELTKVKYVTEGMRKPEFLSAEQKQEIFDLLFKKNRKVTKKKLKEYLFKKF | |
| | ECFDIVEITGLDDRFNASLGTYHDLLKIIKDKDFLDNDENEEILEDIVLTLT | |
| | LFEDREMIKKRLEKYADLFDKKQLKKLKRRHYTGWGRLSKKLINGIRDKQSG | |
| | KTILDYLIDDGFANRNFMQLIHDDSLTFKEEIQKAQVIGDGDSLHEHIANLA | |
| | GSPAIKKGILQTVKVVDELVKVMGRHKPENIVIEMARENQTTQKGQRNSRQR | |
| | LKRIEEAIKELGSKILKEHPVENQQLQNDRLYLYYLQNGRDMYTGEELDIDR | |
| | LSQYDIDHIIPQSFIKDDSIDNRVLVSSAKARGKSDNVPSEEVVKKMKSYWK | |
| | KLLDAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-110 | MDKKYSIGLDIGTNSVGWAVITDEYKVPSKKFKVLGNTDRHSIKKNLIGALL | 70 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVQKLFIQLVQTYNQLFEESPLNEEGVDA | |
| | KAILSARLSKSRRLENLISQFPGEKKNGLFGNLIALSLGLTPNFKSNFDLAE | |
| | DAKLQLSKDTYDEDLENLLGQIGDQYADLFLAAKKLSDAILLSGILTVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VIAKVEKGKAKKLKTVKELVGITIMERSAFEKNPVAFLEDKGYKNIQKDLII | |
| | KLPKYSLFELENGRKRLLASAGELQKGNELALPNKYVTLLYHASNYEKLKGS | |
| | PEDNEQKRLYVEQHKDYFDEILDQIIEFSKRYILADANLEKIKSLYEKNEDS | |
| | SIEELATSFINLLTFTALGAPAAFKFFGTDIDRKRYTSTKEILNATLIHQSI | |
| | TGLYETRIDLSQLGGD | |
| CasEnd-111 | MKKKYSIGLDIGTNSVGWAVVTDEYKVPSKKFKVLGNTDRTSIKKNLLGALL | 71 |
| | FDSGETAEATRLKRTARRRYTRRKNRIRYLQEIFSNEMSKVDDSFFHRLEES | |
| | FLVEEDKKHERHPIFGNIVDEVAYHEKYPTIYHLRKHLADSTEKADLRLIYL | |
| | ALAHIIKFRGHFLIEGDLNIDNSDVQKLFIQLVQTYNNLFEENHLNESGVDA | |
| | KAILTAALSKSRRLENLIAQFPGEKKNGLFGNLIALSLGLTPNFKSNEDLEE | |
| | DAKLQLSKDTYDEDLDNLLAQIGDQYADLFLAAKNLSDAILLSGILTVNDEV | |
| | TKAPLSASMIKRYDEHHQDLTLLKNFVRQQLPEKYKEIFFDESKNGYAGYID | |
| | GGASQEEFYKYIKPILEKMDGAEEFLAKINREDFLRKQRTFDNGSIPHQIHL | |
| | GELHAILRRQEDFYPFLKDNREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMGKPEFLSSEQKEEIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLEKYANLFDDKVLKKLKRRRYTGWGRLSKKLINGIRDKQSG | |
| | KTILDFLKDDGFANRNFMQLIHDDSLTFKEAIQKAQVIGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIREVKIITLKSKLVSDFRKDFEFYKVREINDYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPIAFLEDKGYKEVQKDLII | |
| | KLPKYSLFELENGRKRLLASAGELQKGNELALPSHYVRFLYLAKNYEKLKGK | |
| | EEDDEKKRYYVEQHRDEFDEILEQISEFSERYILADKNLEKILELYNENEDK | |
| | DINELAENFIHLFTFTSLGAPAAFKFFDTTIDRKRYTSTKEILDATLIHQSI | |
| | TGLYETRIDLSQLGGD | |
| CasEnd-112 | MDKKYSIGLDIGTNSVGWAVITDEYKVPSKKFKVLGNTDRQSIKKNLLGALL | 72 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINESGVDA | |
| | KAILSAKLSKSRRLENLIAQLPGEKKNGLFGNLIALSLGLTPNFKSNEDLAE | |
| | DAKLQLSKDTYDDDLENLLGQIGDQYADLFLAAKNLSDAILLSDILTVNDEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPTEKVLPKHSLLYEYFTV | |
| | YNELTKVKYVTEGMRKPEFLSGNQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYANLFDDKVLKQLKRRRYTGWGRLSRKLINGIRDKQSG | |
| | KTILDFLKSDGFANRNFMQLIHDDSLTFKEDIKKAQVSGQGDSLHEQIANLA | |
| | GSPAIKKGILQTVKIVDELVKVMGRHKPENIVIEMARENQTTQKGQKNSRER | |
| | MKRLEEGIKELGSQILKEHPVENTQLQNEKLYLYYLQNGKDMYTGQELDIDR | |
| | LSDYDVDHIIPQSFIKDDSIDNKVLTSSDKARGKSDDVPSEEVVKKMKNYWR | |
| | QLLKAKLISQRKFDNLTKAERGGLSELDKAGFIKRQLVETRQITKHVAQILD | |
| | SRFNTEHDENDKLIRDVKIITLKSKLVSDFRKDFQFYKVREINDYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELVGITIMERSSFEKDPVDFLEAKGYKEVQKDLII | |
| | KLPKYSLFELENGRKRMLASAGELQKGNELALPSKYVTFLYLASHYEKLKGS | |
| | PEDNEQKQLFVEQHKDYFDEILEQISEFSKRYILADKNLEKILSLYNENEDK | |
| | SIEEQAENFINLFTLTNLGAPAAFKFFGTTIDRKRYTSTKEILDSTLIHQSI | |
| | TGLYETRIDLSQLGGD | |
| CasEnd-113 | MDKKYSIGLDIGTNSVGWAVITDEYKVPSKKFKVLGNTDRHSIKKNLIGALL | 73 |
| | FDSGETAEATRLKRTARRRYTRRKNRIRYLQEIFSNEMAKVDDSFFHRLEES | |
| | FLVEEDKKHERHPIFGNIVDEVAYHEKYPTIYHLRKKLADSDEKADLRLIYL | |
| | ALAHMIKFRGHFLIEGDLNSDNSDVQKLFIQLVQTYNQLFEENPINESGVDA | |
| | KAILSARLSKSRRLENLIAQFPGEKKNGLFGNLIALSLGLTPNFKSNFDLAE | |
| | DAKLQLSKDTYDEDLENLLAQIGDQYADLFLAAKNLSDAILLSDILTVNDEI | |
| | TKAPLSASMVKRYDEHHQDLTLLKALVRQQLPEKYKEIFFDQSKNGYAGYID | |
| | GGASQEEFYKYIKPILEKMDGSEEFLAKINREDFLRKQRTFDNGSIPHQIHL | |
| | GELHAILRRQEKYYPFLKDNREKIEKILTFRIPYYVGPLARGNSRFAWMTRK | |
| | SEETITPWNFEEVVDKEASAQKFIERMTNFDKNLPNEKVLPKHSLLYEYFTV | |
| | YNELTKVKYVTEGMRKPEFLSGEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVLKQLKRRRYTGWGRLSRKLINGIRDKQSG | |
| | KTILDFLKSDGFANRNFMQLIHDDSLTFKEEIEKAQVSGQGDSLHEQIANLA | |
| | GSPAIKKGILQTIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VIAKVEKGKAKKLKSVKELVGITIMERSSFEKNPIDFLEAKGYKEIKKDLII | |
| | KLPKYSLFELENGRKRMLASAGELQKGNELALPSKYVTFLYLASHYEKLKGS | |
| | EEDNEQKQLFVEQHKHYFDEIIEQISEFSKRYILADANLEKILSLYNKNRDK | |
| | SIEEQAENFIHLFTFTNLGAPAAFKFFDTTIDRKRYTSTKEILDATLIHQSI | |
| | TGLYETRIDLSQLGGD | |
| CasEnd-114 | MDKKYSIGLDIGTNSVGWAVITDEYKVPSKKFKVLGNTDRHSIKKNLIGALL | 74 |
| | FDSGETAEATRLKRTARRRYTRRKNRICYLQEIFSNEMAKVDDSFFHRLEES | |
| | FLVEEDKRNERHPIFGNIVDEVAYHEKYPTIYHLRKKLADSDEKADLRLIYL | |
| | ALAHMIKFRGHFLIEGDLNSENSDVQKLFIQLVQTYNQTFEESPLNEETVDA | |
| | KAILTARLSKSRRLENLIAQFPGEKKNGLFGNLIALSLGLTPNFKSNFDLAE | |
| | DAKLQFSKDTYDEDLENLLGQIGDEYADLFLAAKNLYDAILLSGILTVNDEI | |
| | TKAPLSASMVKRYDEHQQDLKLLKKLVREQLPEKYKEIFSDKSKNGYAGYID | |
| | GGTSQEEFYKYIKPILEKMDGSEEFLEKINREDFLRKQRTFDNGSIPHQIHL | |
| | GELHAILRRQEKYYPFLKENREKIEKILTFRIPYYVGPLARGNSRFAWMTRK | |
| | SDEKITPWNFEEVVDKEASAQKFIERMTNNDTYLPDEKVLPKHSLLYEKFTV | |
| | YNELTKVKYVTEGMRKPEFLSAEQKQEIVDLLFKKNRKVTVKKLKEYLFKKI | |
| | ECFDSVEISGLEDRFNASLGTYHDLLKIIKDKDFLDNEENEDILEDIVLTLT | |
| | LFEDREMIEERLKKYADLFDKKVLKKLKRRHYTGWGRLSKKLINGIRDKQSG | |
| | KTILDFLIDDGFANRNFMQLIHDDSLTFKEEIKKAQVIGQGDSLHEHIANLA | |
| | GSPAIKKGILQTVKVVDELVKVMGRHKPENIVIEMARENQTTQKGQRNSRQR | |
| | LKRLEEAIKELGSNILKEHPVENSQLQNDRLYLYYLQNGKDMYTGEELDIDR | |
| | LSDYDIDHIIPQSFIKDDSIDNRVLVSSAKARGKSDNVPSEEVVKKMKNYWR | |
| | QLLEAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-115 | MDKKYSIGLDIGTNSVGWAVITDEYKVPSKKFKVLGNTDRHSIKKNLIGALL | 75 |
| | FDSGETAEATRLKRTARRRYTRRKNRIRYLQEIFANEMAKVDDSFFHRLEES | |
| | FLVEEDKKNERHPIFGNIVDEVAYHEKYPTIYHLRKKLVDSDEKADLRLIYL | |
| | ALAHMIKFRGHFLIEGDLNSDNSDVDKLFIQLVQTYNQLFEENPIEAEGVDA | |
| | KAILSERLSKSRRLENLIAQLPGEKKNGLFGNLIALSLGLTPNFKSNFDLAE | |
| | DAKLQLSKDTYDDDLENLLGQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKYIKPILEKMDGTEELLAKLDREDLLRKQRTEDNGSIPHQIHL | |
| | GELHAILRRQEKYYPFLKENREKIEKILTFRIPYYVGPLARGNSRFAWMTRK | |
| | SDETITPWNFEEVVDKGASAEAFIERMTNFDKNLPNEKVLPKHSLLYEYFTV | |
| | YNELTKVKYVTEGMRKPEFLDGEQKKEIVDLLFKKNRKVTVKQLKEDYFKEF | |
| | ECFDSVEISGVEDRFNASLGTYHDLLKIIKDKDFLDNEENEDILEDIVLTLT | |
| | LFEDREMIKERLKKYANLFDDKVMKQLKRRRYTGWGRLSRKLINGIRDKQSG | |
| | KTILDFLKSDGFANRNFMQLIHDDSLTFKDEIEKAQVSGQGESLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDDNDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIEVNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELLGITIMERSAFEKDPVAFLEAKGYKEVKKDLII | |
| | KLPKYSLFELENGRKRMLASAGELQKGNELALPSKYVNFLYLASHYEKLKGS | |
| | EEDNKQKQLFVEQHKEYLDEIIDQISEFSKRVILADANLEKVKSAYEKHRDK | |
| | SIEEQAENIIHLFTLTNLGAPAAFKYFDTTIDRKRYTSTKEVLNATLIHQSI | |
| | TGLYETRIDLSQLGGD | |
| CasEnd-116 | MDKKYSIGLDIGTNSVGWAVITDEYKVPSKKFKVLGNTDRHSIKKNLIGALL | 76 |
| | FDSGETAEATRLKRTARRRYTRRKNRIRYLQEIFSEEMAKVDDSFFHRLEES | |
| | FLVEEDKRHERHPIFGNIVDEVAYHEKYPTIYHLRKKLADSDEKADLRLIYL | |
| | ALAHMIKFRGHFLIEGDLNSDNSDVQKLFIQLVQTYNQLFEENPINEEGVDA | |
| | KAILTARLSKSRRLENLIAQFPGEKKNGLFGNLIALSLGLTPNFKSNFDLAE | |
| | DAKLQLSKDTYDDDLENLLAQIGDQYADLFLAAKNLSDAILLSGILTVNDEI | |
| | TKAPLSASMVKRYDEHHQDLTLLKALVRQQLPEKYKEIFFDQSKNGYAGYID | |
| | GGASQEEFYKYLKPILEKLDGSEEFLDKIEREDFLRKQRTFDNGSIPHQIHL | |
| | GELHAILRRQEKYYPFLKENREKIEKILTFRIPYYVGPLARGNSRFAWMTRK | |
| | SEETITPWNFEEVVDKEASAQAFIERMTNFDKNLPEEKVLPKHSLLYEKFTV | |
| | YNELTKVKYVTEGMRKPEFLSAEQKQEIVDLLFKKNRKVTVKQLKEYYFKKI | |
| | ECFDSVEISGVEDRFNASLGTYHDLLKIIKDKDFLDNEENEDILEDIVLTLT | |
| | LFEDREMIEKRLKKYAHLFDDKVLKKLKRRRYTGWGRLSKKLINGIRDKQSG | |
| | KTILDFLISDGFANRNFMQLIHDDSLTFKEEIEKAQVIGQGDSLHELIANLA | |
| | GSPAIKKGILQTIKIVDELVKVMGRHEPENIVIEMARENQTTQKGQKNSRER | |
| | MKRLEEGIKELGSDILKEHPVENTQLQNEKLYLYYLQNGKDMYTGEELDIDR | |
| | LSDYDVDHIVPQSFLKDDSIDNKVLVSSEKARGKSDNVPSEEVVKKMKNYWK | |
| | QLLEAKLITQRKFDNLTKAERGGLTELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNEETGEIVWDKGKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKNPIAFLEAKGYKNVQKDLII | |
| | KLPKYSLFELENGRKRMLASAGELQKGNELALPNKYVTLLYLASHYEKLKGS | |
| | PEDNEQKQLFVEQHKEYFDEIIEQISEFSKRYILADANLEKIKSLYEKNRDK | |
| | SIEEQAENFINLLTFTALGAPAAFKFFDTTIDRKRYTSTKEILNATLIHQSI | |
| | TGLYETRIDLSQLGGD | |
| CasEnd-117 | MDKKYSIGLDIGTNSVGWAVITDEYKVPSKKFKVLGNTDRHSIKKNLIGALL | 77 |
| | FDSGETAEATRLKRTARRRYTRRKNRICYLQEIFSNEMAKVDDSFFHRLEES | |
| | FLVEEDKKNERHPIFGNIVDEVAYHEKYPTIYHLRKKLADSDEKADLRLIYL | |
| | ALAHMIKFRGHFLIEGDLNSDNSDVDKLFIQLVQTYNQLFEENPINEEGVDA | |
| | KAILSARLSKSRRLENLIAQFPGEKKNGLFGNLIALSLGLTPNFKSNEDLAE | |
| | DAKLQLSKDTYDEDLENLLGQIGDQYADLFLAAKNLSDAILLSDILTVNDEI | |
| | TKAPLSASMVKRYDEHHQDLTLLKALVRQQLPEKYKEIFFDQSKNGYAGYID | |
| | GGASQEEFYKYIKPILEKMDGSEEFLDKLEREDFLRKQRTFDNGSIPHQIHL | |
| | GELHAILRRQEKYYPFLKDNREKIEKILTFRIPYYVGPLARGNSRFAWMTRK | |
| | SDETITPWNFEEVVDKEASAQKFIERMTNFDKNLPNEKVLPKHSLLYEYFTV | |
| | YNELTKVKYVTEGMRKPEFLSGEQKQEIVDLLFKKNRKVTVKQLKEDYFKKF | |
| | ECFDSVEISGVEDRFNASLGTYHDLLKIIKDKDFLDNEENEDILEDIVLTLT | |
| | LFEDREMIEERLKKYADLFDKKVLKQLKRRRYTGWGRLSKKLINGIRDKQSG | |
| | KTILDFLKSDGFANRNFMQLIHDDSLTFKEEIQKAQVSGKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VIAKVEKGKAKKLKTVKELVGITIMERSSFEKNPIDFLEAKGYKEIKKDLII | |
| | KLPKYSLFELENGRKRLLASAGELQKGNELALPNKYVTFLYLASHYEKLKGS | |
| | PEDNEQKQLFVEQHKHYFDEIIEQISEFSKRVILADANLEKILSLYNKNRDK | |
| | SIEEQAENIIHLFTFTNLGAPAAFKFEDTTIDRKRYTSTKEILDATLIHQSI | |
| | TGLYETRIDLSQLGGD | |
| CasEnd-118 | MKKKYSIGLDIGTNSVGWAVITDEYKVPSKKFKVLGNTDRQSIKKNLIGALL | 78 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSENSDVQKLFIQLVQTYNQLFEENPLNESGVDA | |
| | KAILSAKLSKSRRLENLIAQLPGEKKNGLFGNLIALSLGLTPNFKSNEDLSE | |
| | DAKLQLSKDTYDDDLDNLLGQIGDQYADLFLAAKNLSDAILLSDILRVNTEI | |
| | TKAPLSASMIKRYDEHHQDLTLLKALVRQQLPEKYKEIFFDESKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMGKPEFLSGEQKKEIVDLLFKKNRKVTVKQLKEFYFKEI | |
| | ECFDSVEISGVEDRFNASLGTYHDLLKIIKDKDFLDNEENEDILEDIVLTLT | |
| | LFEDREMIEERLKKYANLFDDKVMKQLKRRRYTGWGRLSRKLINGIRDKQSG | |
| | KTILDFLKSDGFANRNFMQLIHDDSLTFKEEIKKAQVKGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGQELDINR | |
| | LSDYDVDHIVPQSFLKDDSIDNKVLTRSEKARGKSDNVPSEEVVKKMKSYWR | |
| | QLLKAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEIKLANGEIRKRPLIETNEETGEIVWDKERDFATVRKVLSMPQV | |
| | NIVKKTEVQTGGFSKESILPKRESDKLIARKKDWDPKKYGGFDSPTVAYSVL | |
| | VVAKVEKGKSKKLKTVKELLGITIMERSSFEKDPVAFLEAKGYKDVKKDLII | |
| | KLPKYSLFELENGRKRMLASAGELQKGNELALPSKYVNFLYLASHYEKLKGK | |
| | PEDEEQKQLFVEQHKHYLDEIIEQISEFSERVILADKNLEKVLSAYSKHRDK | |
| | SISEQAENIIHLFTLTNLGAPAAFKYFDTTIDRKRYTSTKEVLNATLIHQSI | |
| | TGLYETRIDLSQLGGD | |
| CasEnd-119 | MDKKYSIGLDIGTNSVGWAVITDEYKVPSKKFKVLGNTDRHSIKKNLIGALL | 79 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENSDVQKLFIQLVQTYNQLFEENPINEEGVDA | |
| | KAILSAKLSKSRRLENLIAQLPGEKKNGLFGNLIALSLGLTPNFKSNEDLAE | |
| | DAKLQLSKDTYDEDLENLLAQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKYIKPILEKMDGTEELLEKINREDFLRKQRTFDNGSIPHQIHL | |
| | GELHAILRRQEDYYPFLKENREKIEKILTFRIPYYVGPLARGNSRFAWMTRK | |
| | SEETITPWNFEEVVDKEASAQAFIERMTNFDKNLPTEKVLPKHSLLYEKFTV | |
| | YNELTKVKYVTEGMGKPEFLSAEQKQEIVDLLFKKNRKVTVKQLKEDYFKEI | |
| | ECFDSVEISGVEDRFNASLGTYHDLLKIIKDKEFLDNEENEDILEDIVLTLT | |
| | LFEDREMIEERLKKYADLFDKKVLKQLKRRRYTGWGRLSKKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRLEEGIKELGSQILKEHPVENTQLQNDKLYLYYLQNGKDMYTGEELDIDR | |
| | LSDYDVDHIIPQSFIKDDSIDNKVLTSSEKARGKSDNVPSEEVVKKMKNYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSSFEKNPIAFLEAKGYKNIQEDLII | |
| | KLPKYSLFELENGRKRLLASAGELQKGNELALPNKYVTLLYHASHYEKLKGS | |
| | PEDEEKKLLYVEQHRSYFDEILEQISEFSKRYILADKNLEKILELYNKFRDK | |
| | SIEEQAESFINLFTFTALGAPAAFKFFDTTIDRKRYTSTKEILNSTLIHQSI | |
| | TGLYETRIDLSQLGGD | |
| CasEnd-120 | MKKSYSIGLDIGTNSVGWAVITDEYKVPSKKFKVLGNTDRKSIKKNLLGALL | 80 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKKLADSDEKADLRLIYL | |
| | ALAHIIKFRGHFLIEGDLNSDNSDVQKLFIQLVQTYNQLFEESVINEIGVDA | |
| | KAILSARLSKSRRLENLIAQFPGEKRNGLFGNLIALSLGLTPNFKSNEDLAE | |
| | DAKLQFSKDTYDEDLDNLLAQIGDQYADLFLAAKNLSDAILLSGILTVNSES | |
| | TKAPLSASMIKRYDEHHQDLTLLKALVREQLPEKYKEIFFDQSKNGYAGYID | |
| | GGASQEEFYKYIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKGASAQAFIERMTNFDKNLPNEKVLPKHSLLYEYFTV | |
| | YNELTKVKYITEGMRKPEFLSGEQKKAIVDLLFKKNRKVTVKQLKEDYFKKI | |
| | ECFDSVEISGVEDRFNASLGTYHDLLKIIKDKDFLDNEENEDILEDIILTLT | |
| | LFEDREMIEERLKKYAHLFDKKVLKQLKRRRYTGWGRLSGKLINGIRDKQSG | |
| | KTILDFLKSDGFANRNFMQLIHDDSLTFKEIIQKAQVSGQGDSLHEHIANLA | |
| | GSPAIKKGILQSIKIVDELVKVMGRHDPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIREVKIVTLKSKLVSDFRKDFGFYKVREINDYHHAHDAY | |
| | LNAVVGTALIKKYPKLESEFVYGDYKVYDVRKMIAKSEQEIGKATAKYFFYS | |
| | NIMNFFKTEITLANGEVRKRPMIETNEETGEIVWDKTKDFATVRKVLSMPQV | |
| | NIVKKTEVQTGGFSKESILPKRNSDKLIARKKDWDTKKYGGFDSPTVAYSVL | |
| | VVADVEKGKAKKLKTVKELVGITIMERSSFEKNPVAFLETKGYQNVQKDLII | |
| | KLPKYSLFELENGRKRLLASAGELQKGNELALPQKYVTLLYHASHYEKLKGK | |
| | SEDEEHKLEYVKQHRDEFDEILDQIEEFSKRYILADKNLEKIKELYAENRDS | |
| | SINELAENFIHLFTFTSLGAPAAFKFFDKTIDRKRYTSTTEILNATLIHQSI | |
| | TGLYETRIDLSQLGGD | |
| CasEnd-121 | MKKKYSIGLDIGTNSVGWAVITDEYKVPSKKFKVLGNTDRQSIKKNLIGALL | 81 |
| | FDSGETAEATRLKRTARRRYTRRKNRIRYLQEIFAEEMNKVDDSFFHRLEES | |
| | FLVEEDKRNERHPIFGNIVDEVAYHEKYPTIYHLRKKLADSTEKADLRLIYL | |
| | ALAHIIKFRGHFLIEGDLNSDNSDVQKLFIQLVQTYNQLFEENPLNEEGVDA | |
| | KAILSAKLSKSRRLENLIALFPTEKKNGLFGNLIALSLGLTPNFKSNFDLSE | |
| | DAKLQLSKDTYDEDLENLLAQIGDQYADLFLAAKNLSDAILLSGILTVNDEI | |
| | TKAPLSASMIKRYDEHHQDLTLLKALVRQQLPEKYKEIFFDQSKNGYAGYID | |
| | GGASQEDFYKYIKPILEKMDGTEEFLEKINREDFLRKQRTFDNGSIPHQIHL | |
| | GELHAILRRQEEYYPFLKENQEKIEKILTFRIPYYVGPLARGNSRFAWMTRK | |
| | SDETITPWNFEEVVDKEASAQAFIERMTNFDKNLPTEKVLPKHSLLYEKFTV | |
| | YNELTKVKYVTEGMGKPEFLSAEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDKKVLKQLKRRRYTGWGRLSKKLINGIRDKQSG | |
| | KTILDFLISDGFANRNFMQLIHDDSLTFKEEIKKAQEIGQGDSLHEQIANLA | |
| | GSPAIKKGILQTVKIVDEIVKVMGRHKPENIVIEMARENQTTQKGQKNSRER | |
| | LKRIEEGLKELGSQILKEHPVENTQLQNDKLYLYYLQNGKDMYTGEELDIDR | |
| | LSDYDVDHIIPQSFIKDDSIDNKVLTSSDKARGKSDDVPSEEVVKKMKNYWR | |
| | QLLNAKLISQRKFDNLTKAERGGLSELDKAGFIKRQLVETRQITKHVAQILD | |
| | SRFNTERDENDKLIRDVKIITLKSKLVSDFRKDFGFYKVREINDYHHAHDAY | |
| | LNAVVGTALIKKYPKLESEFVYGDYKVYDVRKMIAKSEQEIGKATAKRFFYS | |
| | NIMNFFKTDIKLANGEIRKRPLIETNEETGEIVWDKEKDFATVRKVLSMPQV | |
| | NIVKKTEVQTGGFSKESILPKRDSDKLIARKKDWDTKKYGGFDSPTVAYSVL | |
| | VVAKVEKGKTKKLKTVKELVGITIMERSRFEKDPVAFLEAKGYQEIQEDLII | |
| | KLPKYSLFELENGRKRLLASAGELQKGNELALPSKYVTFLYLASHYEKLKGS | |
| | PEDNEKKLEYVEQHRYYFDEIFEQISEFSKRYILADKNLEKILELYNQHRDA | |
| | PIEELAESFINLFTFTALGAPAAFKFFGTTIDRKRYTSTKEILNSTLIHQSI | |
| | TGLYETRIDLSQLGGD | |
| CasEnd-122 | MDKKYSIGLDIGTNSVGWAVITDEYKVPSKKFKVLGNTDRQSIKKNLIGALL | 82 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENSDVDKLFIQLVQTYNQLFEENPINESGVDA | |
| | KAILSAKLSKSRRLENLIAQLPGEKRNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVREQLPEKYKEIFFDQSKNGYAGYID | |
| | GGASQEEFYKYIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPTEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIEERLKKYAHLFDKKVLKQLKRRRYTGWGRLSRKLINGIRDKQSG | |
| | KTILDFLKSDGFANRNFMQLIHDDSLTFKEAIQKAQVSGQGDSLHEQIANLA | |
| | GSPAIKKGILQTIKIVDELVKVMGRHKPENIVIEMARENQTTQKGQKNSRER | |
| | MKRLEEGIKELGSQILKEHPVENTQLQNEKLYLYYLQNGKDMYTGQELDIDR | |
| | LSDYDVDHIIPQSFIKDDSIDNKVLTSSDKARGKSDNVPSEEVVKKMKNYWR | |
| | QLLNAKLISQRKFDNLTKAERGGLSELDKAGFIKRQLVETRQITKHVAQILD | |
| | SRMNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKDPVDFLEAKGYKEVQKDLII | |
| | KLPKYSLFELENGRKRMLASAGELQKGNELALPSKYVTFLYLASHYEKLKGS | |
| | PEDEEKKQLYVEQHKHYFDEILEQISEFSKRYILADANLEKILSLYNQFEDK | |
| | PIEEQAESFINLFTLTALGAPAAFKFFDTTIDRKRYTSTKEILDSTLIHQSI | |
| | TGLYETRIDLSQLGGD | |
| CasEnd-123 | MKKKYSIGLDIGTNSVGWAVITDEYKVPSKKFKVLGNTDRQSIKKNLIGALL | 83 |
| | FDSGETAEATRLKRTARRRYTRRKNRIRYLQEIFSEEMSKVDDSFFHRLEES | |
| | FLVEEDKRHERHPIFGNIVDEVAYHEKYPTIYHLRKKLADSTQKADLRLIYL | |
| | ALAHIIKFRGHFLIEGDLNPENSDVQKLFIQLVQTYNQLFEENILNESRVDA | |
| | KAILSARLSKSRRLENLIAQLPGEKKNGLFGNLIALSLGLTPNFKSNFDLSE | |
| | DAKLQLSKDTYDEDLDNLLAQIGDEYADLFLAAKNLSDAILLSGILTVNDES | |
| | TKAPLSASMIKRYDEHHQDLTLLKALVRQQLPEKYKEIFFDQSKNGYAGYID | |
| | GGASQEEFYKYIKPILEKMDGTEYLLAKINREDFLRKQRTFDNGSIPHQIHL | |
| | GELHAILRRQEDFYPFLKENREKIEKILTFRIPYYVGPLARGNSRFAWLTRK | |
| | SEETITPWNFEEVVDKEASAQAFIERMTNFDKNLPNEKVLPKHSLLYEMFTV | |
| | YNELTKVKYVTEGMRKPEFLSSGQKEEIVDLLFKKNRKVTVKQLKEFYFKKI | |
| | ECFSSVEISGVEDRFNASLGTYHDLLKIIKDKDELDNEENEDILEDIVLTLT | |
| | LFEDREMIEERLKKYAHLFDKKVLKKLKRRRYTGWGRLSRKLINGIRDKQSG | |
| | KTILDFLKDDGFANRNFMQLIHDDSLTFKEDIQKAQVKGEGESLHEQIANLA | |
| | GSPAIKKGILQSIKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIREVKIITLKSKLVSDFRKDFQFYKVREINNYHHAHDAY | |
| | LNAVVGTALIKKYPKLESEFVYGDYKVYDVRKMIAKSDQEIGKATAKYFFYS | |
| | NIMNFFKTEITLANGEVRKRPLIETNEETGEIVWDKTKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKDPVAFLESKGYKNIQKDLII | |
| | KLPKYSLFELENGRKRLLASAGELQKGNELALPQHYVTLLYLASRYEKLKGK | |
| | PEDNEKKRNYVDQHRQEFDEILDQISEFSKRYILADANLDKILSLYNENRDA | |
| | SISELAENFIHLFTFTSLGAPAAFKFFDSDIDRKRYTSTKEVLNATLIHQSI | |
| | TGLYETRIDLSKLGGD | |
| CasEnd-124 | MDKKYSIGLDIGTNSVGWAVITDEYKVPSKKFKVLGNTDRHSIKKNLIGALL | 84 |
| | FDSGETAEATRLKRTARRRYTRRKNRIRYLQEIFSEEMAKVDDSFFHRLEES | |
| | FLVEEDKRNERHPIFGNIVDEVAYHEKYPTIYHLRKKLADSTEKADLRLIYL | |
| | ALAHIIKFRGHFLIEGDLNSENSDVQKLFIQLVQTYNNLFEENPITEEGVDA | |
| | KAILSAKLSKSRRLENLIAEFPGEKKNGLFGNLIALSLGLTPNFKSNEDLSE | |
| | DAKLQFSKDTYDEDLDNLLAQIGDQYADLFLAAKNLYDAILLSGILTVNTEI | |
| | TKAPLSASMIKRYDEHHQDLTLLKKLVREQLPEKYKEIFFDESKNGYAGYID | |
| | GGASQEEFYKYIKPILEKMDGSEYLLVKLEREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQSFIERMTNFDKNLPNEKVLPKHSLLYEYFTV | |
| | YNELTKVKYVTEGMRKPAFLSGEQKKEIVDLLFKTNRKVTVKQLKEDLFKKI | |
| | ECFDSVEISGVEDRFNASLGTYHDLLKIIKDKDFLDNEENEDILEDIILTLT | |
| | LFEDREMIEERLDKYAHLFDKKVLKKLKRRRYTGWGRLSKKLINGIRDKQSG | |
| | KTILDFLKDDGFANRNFMQLIHDDSLTFKEEIKKAQVIGQTDSLHEVIADLA | |
| | GSPAIKKGILQSIKIVDELVKVMGRHNPENIVIEMARENQTTQKGQKNSRER | |
| | MKRLEESIKELGSQILKEHPVENTQLQNDKLYLYYLQNGKDMYTGEELDIDR | |
| | LSDYDVDHIIPQSFIKDDSIDNKVLTSSDEARGKSDNVPSEEVVKKMKSYWR | |
| | QLLNAKLISQRKFDNLTKAERGGLSELDKAGFIKRQLVETRQITKHVAQILD | |
| | SRFNTKYDENDKLIREVKIITLKSKLVSDFRKDFQFYKVREINDYHHAHDAY | |
| | LNAVVGTALIKKYPKLESEFVYGDYKVYNSRKMIAKSEQEIGKATAKYFFYS | |
| | NIMNFFKSDITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPIAFLEAKGYKNVQKDLII | |
| | KLPKYSLFELENGRKRLLASAGELQKGNELALPNKYVTLLYHAKHYEKLKGS | |
| | PEDNEQKMEYVEQHKYYFDEILEQISEFSERYILADKNLEKIKSLYKENADK | |
| | DIEELASSFINLFTFTALGAPAAFKFFDTTIDRKRYTSTTEILDATLIHQSI | |
| | TGLYETRIDLSQLGGD | |
| CasEnd-125 | MKKKYSIGLDIGTNSVGWAVITDEYKVPSKKFKVLGNTDRQSIKKNLLGALL | 85 |
| | FDSGETAEATRLKRTARRRYTRRKNRIRYLQEIFSEEMAKVDDSFFHRLEES | |
| | FLVEEDKRHERHPIFGNIVDEVAYHEKYPTIYHLRKELADSTEKADLRLIYL | |
| | ALAHMIKFRGHFLIEGDLNSENSDVQKLFIQLVQTYNQLFEENPINEETVDA | |
| | KAILSAKLSKSRRLENLIAQLPGEKKNGLFGNLIALSLGLTPNFKSNFGLSE | |
| | DAKLQLSKDTYDEDLENLLAQIGDQYADLFLAAKNLSDAILLSDILTVNDEI | |
| | TKAPLSASMIKRYDEHHQDLTLLKALVRQQLPEKYKEIFFDESKNGYAGYID | |
| | GGASQEEFYKFIKPILEKMDGAEYLLAKINREDFLRKQRTFDNGSIPHQIHL | |
| | GELHAILRRQEEFYPFLKENREKIEKILTFRIPYYVGPLARGNSRFAWMTRK | |
| | SDETITPWNFEEVVDKEASAQAFIERMTNFDKNLPNEKVLPKHSLLYEKFTV | |
| | YNELTKVKYVTEGMRKPEFLSSEQKQEIVDLLFKKNRKVTVKQLKEFYFKKI | |
| | ECFDSVEISGVEDRFNASLGTYHDLLKIIKDKAFLDNEENEDILEDIVLTLT | |
| | LFEDREMIEERLEKYANLFDDKVLKKLKRRRYTGWGRLSKKLINGIKDKQSG | |
| | KTILDFLKSDGFANRNFMQLIHDDSLTFKEEIKKAQVKGQGDSLHEQIANLA | |
| | GSPAIKKGILQSVKIVDELVKVMGRHKPENIVIEMARENQTTQKGQKNSRER | |
| | MKRLEESIKELGSKILKEHPVENTQLQNDKLYLYYLQNGKDMYTGQELDIDR | |
| | LSDYDVDHIIPQSFIKDDSIDNKVLTSSEKARGKSDNVPSEEVVKKMKSYWR | |
| | QLLNAKLISQRKFDNLTKAERGGLSELDKAGFIKRQLVETRQITKHVAQILD | |
| | SRFNTKYDENDKLIREVKIITLKSKLVSDFRKDFQFYKVREINNYHHAHDAY | |
| | LNAVVGTALIKKYPKLESEFVYGDYKVYDVYKMVAKSEQEIGKATAKRFFYS | |
| | NIMNFFKTEIKLANGTIRKRPLIETNEETGEIVWDKTKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKNPVAFLEDKGYQNIQEDLII | |
| | KLPKYSLFELENGRKRLLASAGELQKGNELALPQKYVTFLYLASHYEKLKGK | |
| | PEDEEKKQLFVEQHKSYFDEIMDQISEFSERYILADANLEKILSLYNEFEDK | |
| | SIEEQAESFINLFTFTALGAPAAFKFFDTTIDRKRYTSTKEILNATLIHQSI | |
| | TGLYETRIDLSQLGGD | |
| CasEnd-126 | MDKKYSIGLDIGTNSVGWAVITDEYKVPSKKFKVLGNTDRHSIKKNLIGALL | 86 |
| | FDSGETAEATRLKRTARRRYTRRKNRICYLQEIFSNEMAKVDPSFFERLEES | |
| | FLVEEDKKTSRHPIFGNIVEEVAYHEKYPTIYHLRKKLVDSKEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTSD | |
| | TNAPLSASMIKRYDDHHQDLTKLKELVRKELPEKYKEIFFDQNKNGYAGYID | |
| | GGATQEEFYKYIKPILESMKGTKELLEKLEKRDLLRKQRTFDNGSIPHQIHL | |
| | GELRAILKRQEKFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDERKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKKPVGKKKKLVEVKELLGITIMERSKFEKDPLGFLKEKGYEDVKMDKII | |
| | KLPKYSLFELGNGRKRMLASAGELQKGNELALPSEYVNFLYLASNYEKLKGS | |
| | PEEIKEKQKYVEENKSYLDEIIKQISEFSKRVIKADANLKKVLEAYEKHKDK | |
| | PISEQAENIIHLFTLTALGAPAAFKYFDETIDRKRYTSTKEVLDATLIHQSI | |
| | TGLYETRIDLKFLGGD | |
| CasEnd-127 | MDKKYSIGLDIGTNSVGWAVITDEYKVPSKKFKVLGNTDRHSIKKNLIGALL | 87 |
| | FDSGETAEATRLKRTARRRYTRRKNRICYLQEIFSNEMAKVDDSFFHRLEES | |
| | FLVEEDKRHERHPIFGNIVDEVAYHEKYPTIYHLRKKLADSTEKADLRLIYL | |
| | ALAHIIKFRGHFLIEGDLNPENSDVDKLFIQLVQTYNQLFEENPINEEGVDA | |
| | KAILSAKLSKSRRLENLIAQLPGEKRNGLFGNLIALSLGLTPNFKSNEDLAE | |
| | DAKLQLSKDTYDEDLDNLLAQIGDQYADLFLAAKNLSDAILLSDILTVNDEI | |
| | TKAPLSASMIKRYDEHHQDLTLLKALVRQQLPEKYKEIFFDQSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMINEDKNLPEEKVLPKHSLLYEYFTV | |
| | YNELTKVKYVTEGMGKPEFLSGEQKEEIVDLLFKKNRKVTVKQLKEFYFKEI | |
| | ECFDSVEISGVEDRFNASLGTYHDLLKIIKDKDFLDNEENEDILEDIVLTLT | |
| | LFEDREMIEERLKKYADLFDKKVLKQLKRRRYTGWGRLSKKLINGIRDKQSG | |
| | KTILDFLKSDGFANRNFMQLIHDDSLTFKEEIKKAQVKGQGDSLHEHIANLA | |
| | GSPAIKKGILQTVKVVDELVKVMGRHKPENIVIEMARENQTTQKGQKNSRER | |
| | MKRLEEGIKELGSQILKEHPVENTQLQNEKLYLYYLQNGKDMYTGQELDIDR | |
| | LSDYDVDHIIPQSFIKDDSIDNKVLTSSEKARGKSDNVPSEEVVKKMKSYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRESDKLIARKKDWDPKKYGGFDSPTVAYSVL | |
| | VVAKVEKGKAKKLKTVKELVGITIMERSSFEKDPVAFLEAKGYKEVQKDLII | |
| | KLPKYSLFELENGRKRLLASAGELQKGNELALPQKYVTFLYLASHYEKLKGS | |
| | PEDEEQKQLYVEQHRHYFDEILEQISEFSERYILADKNLEKILSLYNKFEDL | |
| | SIKEQAESIINLFTLTALGAPAAFKFFDTTIDRKRYTSTKEILNSTLIHQSI | |
| | TGLYETRIDLSQLGGD | |
| CasEnd-128 | MDKKYSIGLDIGTNSVGWAVITDEYKVPSKKFKVLGNTDRHSIKKNLIGALL | 88 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKELADSTEKADLRLIYL | |
| | ALAHIIKFRGHFLIEGDLNSENSDVQKLFIQLVQTYNQLFEENHLNEEGVDA | |
| | KAILSAKLSKSRRLENLIAQLPGEKRNGLFGNLLALSLGLTPNFKSNFGLSE | |
| | DAKLQLSKDTYDEDLENLLAQIGDQYADLFVAAKNLSDAILLSGILTVNDES | |
| | TKAPLSASMIKRYDEHHQDLTLLKALVRQQLPEKYKEIFFDESKNGYAGYID | |
| | GGASQEEFYKYIKPILEKMDGTEEFLAKINREDFLRKQRTFDNGSIPHQIHL | |
| | GELHAILRRQEEFYPFLKENREKIEKILTFRIPYYVGPLARGNSRFAWMTRK | |
| | SDETITPWNFEEVVDKEASAQAFIERMTNFDKNLPSEKVLPKHSLLYEYFTV | |
| | YNELTKVKYVTEGMGKPEFFSGEQKQEIVDLLFKKNRKVTVKKLKEFLFKEI | |
| | ECFDIVEISGVEDAFNASLGTYHDLLKIIKDKDFLDNEENEDILEDIVLTLT | |
| | LFEDREMIEKRLKKYADLFDKKVLKQLKRRRYTGWGRLSKKLINGIRDKQSG | |
| | KTILDFLKSDGFANRNFMQLIHDDSLTFKEEIKKAQVKGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-129 | MDKKYSIGLDIGTNSVGWAVITDEYKVPSKKFKVLGNTDRHSIKKNLIGALL | 89 |
| | FDSGETAEATRLKRTARRRYTRRKNRIRYLQEIFSEEMAKVDDSFFHRLEES | |
| | FLVEEDKKHERHPIFGNIVEEVAYHEKYPTIYHLRKKLADSTEKADLRLIYL | |
| | ALAHMIKFRGHFLIEGDLNSDNSDVQKLFIQLVQTYNQLFEENPLNESGVDA | |
| | KAILSARLSKSRRLENLIAQFPGEKKNGLFGNLIALSLGLTPNFKSNEDLAE | |
| | DAKLQLSKDTYDEDLENLLAQIGDQYADLFLAAKNLSDAILLSDILTVNSEI | |
| | TKAPLSASMVKRYDEHHQDLTLLKALVRQQLPEKYKEIFFDQSKNGYAGYID | |
| | GGASQEEFYKYIKPILEKVDGSEEFLDKIEREDFLRKQRTFDNGSIPHQIHL | |
| | QELHAILRRQEDFYPFLKENREKIEKILTFRIPYYVGPLARGNSRFAWMTRK | |
| | SDETITPWNFEEVVDKEASAQSFIERMTNFDKNLPNEKVLPKHSLLYEYFTV | |
| | YNELTKVKYVTEGMRKPEFLSGEQKQEIVDLLFKKNRKVTVKQLKEFLFKEF | |
| | ECFDSVEISGVEDRFNASLGTYHDLLKIIKDKDFLDNEENEDILEDIVLTLT | |
| | LFEDREMIEERLKKYADLFDDKVLKQLKRRRYTGWGRLSKKLINGIRDKQSG | |
| | KTILDFLKADGFANRNFMQLIHDDSLTFKEEIQKAQVSGQGESLHELIANLA | |
| | GSPAIKKGILQTIKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQLYKVREINNYHHAHDAY | |
| | LNAVVGTALIKKYPKLESEFVYGDYKVYDVRKMIAKSEQEIGKATAKYFFYS | |
| | NIMNFFKTEVTLANGEIRKRPLIETNGETGEIVWDKEKDFATVRKVLSMPQV | |
| | NIVKKTEVQTGGFSKESILPKRNSDKLIARKKDWDPKKYGGFDSPTVAYSVL | |
| | VIAKVEKGKAKKLKTVKELVGITIMERSSFEKNPIAFLEAKGYKEVKKDLII | |
| | KLPKYSLFELENGRKRMLASAGELQKGNELALPQKYVTLLYLASHYEKLKGS | |
| | PEDNEQKQLFVEQHKEYFDEIIEQISEFSKRYILADANLEKIKSLYEKNRDA | |
| | TIEEQAENFIHLLTFTALGAPAAFKFFDTTIDRKRYTSTKEILNATLIHQSI | |
| | TGLYETRIDLSQLGGD | |
| CasEnd-130 | MDKKYSIGLDIGTNSVGWAVITDEYKVPSKKFKVLGNTDRHSIKKNLIGALL | 90 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINESGVDA | |
| | KAILSAKLSKSRRLENLIAQLPGEKKNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKYIKPILEKMDGTEYLLAKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMRKPEFLSGEQKEEIVDLLFKKNRKVTVKQLKEFLFKEI | |
| | ECFDSVEISGVEDRFNASLGTYHDLLKIIKDKDFLDNEENEDILEDIILTLT | |
| | LFEDKEMIEERLKKYANLFDKKVLKQLKRRRYTGWGRLSKKLINGIRDKQSG | |
| | KTILDFLKSDGFANRNFMQLIHDDSLSFKEAIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKVEKGKSKKLKTVKELVGITIMERSSFEKDPVKFLEAKGYKEIQKDLII | |
| | KLPKYSLFELENGRKRMLASAGELQKGNELALPSKYVTLLYHASHYEKLKGK | |
| | PEDEEQKQEYVEQHNHYFDEIFEQISEFSERYILADKNLEKILSLYSNNRDK | |
| | SISEQAESFINLFTLTALGAPAAFKFFDTTIDRKRYTSTKEILDATLIHQSI | |
| | TGLYETRIDLSQLGGD | |
| CasEnd-131 | MKKKYSIGLDIGTNSVGWAVITDEYKVPSKKFKVLGNTDRHSIKKNLIGALL | 91 |
| | FDSGETAEATRLKRTARRRYTRRKNRICYLQEIFSEEMAKVDDSFFHRLEES | |
| | FLVEEDKKHERHPIFGNIVDEVAYHENYPTIYHLRKELADSDEKADLRLIYL | |
| | ALAHIIKFRGHFLIEGDLNSENSDVQKLFIQLVQTYNQLFEENPINEEGVDA | |
| | KAILTAKLSKSRRLENLIAQLPGEKKNGLFGNLIALSLGLTPNFKSNFELSE | |
| | DAKLQLSKDTYDEDLDNLLGQIGDQYADLFLAAKNLSDAILLSDILTVNDEI | |
| | TKAPLSASMIKRYDEHHQDLTLLKALVREQLPEKYKEIFFDKSKNGYAGYID | |
| | GGASQEEFYKYIKPILEKLDGSEELLEKINREDFLRKQRTFDNGSIPHQIHL | |
| | GELHAILRRQEVFYPFLKENQEKIEKILTFRIPYYVGPLARGNSRFAWMTRK | |
| | SDETITPWNFEEVVDKGASAQAFIERMTNFDKNLPNEKVLPKHSLLYEYFTV | |
| | YNELTKVKYITEGMGKPEFLDGEQKKEIVDLLFKKNRKVTVKQLKEDLFKKI | |
| | ECFDSVEISGVEDRFNASLGTYHDLLKIIKDKDFLDNEENEDILEDIVLTLT | |
| | LFEDREMIEERLKKYANLFDKKVLKQLKRRRYTGWGRLSKKLINGIRDKQSG | |
| | KTILDFLKSDGFANRNFMQLIHDDSLTFKEEIKKAQVIGQGDSLHEQIANLA | |
| | GSPAIKKGILQTVKIVDEIVKVMGRHAPENIVIEMARENQTTQKGQKNSRER | |
| | MKRIEEVLKKLGSQILKEHPVENTQLQNEKLYLYYLQNGKDMYTGEELDIDR | |
| | LSDYDVDHIIPQSFIKDDSIDNKVLTSSKEARGKSDNVPSEEVVKKMKSYWR | |
| | QLLNAKLISQRKFDNLTKAERGGLSELDKAGFIKRQLVETRQITKHVAQILD | |
| | SRFNTERDENDKLIRNVKIITLKSKLVSDFRKDFGFYKVREINDYHHAHDAY | |
| | LNAVVGTALIKKYPKLESEFVYGDYKVYDSRKMIAKSEQEIGKATAKMFFYS | |
| | NIMNFFKTEIKLANGEIIKRPVIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPIAFLEAKGYQHIRKDLII | |
| | KLPKYSLFELENGRKRLLASAGELQKGNELALPNKYVTFLYLASHYEKLKGK | |
| | PEDEEEKRLYVEQHKHYFDEILDQISEFSKRYILADKNLEKILDLYNKHEDY | |
| | SINELASNFLNLFTLTSLGAPAAFKFFDTTIDRKRYTSTKEILNSTLIHQSI | |
| | TGLYETRIDLSQLGGD | |
| CasEnd-132 | MDKKYSIGLDIGTNSVGWAVITDEYKVPSKKFKVLGNTDRHSIKKNLIGALL | 92 |
| | FDSGETAEATRLKRTARRRYTRRKNRICYLQEIFSNEMAKVDDSFFHRLEES | |
| | FLVPEDKRHERHPIFGNIVDEVAYHEKYPTIYHLRKKLADSDEKADLRLIYL | |
| | ALAHMIKFRGHFLIEGDLNSDNSDVNKLFKQLVQTYNQLFEENPINEETVDA | |
| | KAILSEKLSKSRRLENLIAQFPGEKKNGLFGNLLALSLGLTPNFKSNEDLAE | |
| | DAKLQLSKDTYDEDLENLLGQIGDQYADLFLAAKNLYDAILLSDILTVNDES | |
| | TKAPLSASMVKRYEEHHQDLKLLKKLVREQLPEKYKEIFSDKSKNGYAGYID | |
| | GGTSQEEFYKYIKPILEKVDGSEEFLEKIEREDFLRKQRTFDNGSIPHQIHL | |
| | GELHAILRRQEEYYPFLKENREKIEKILTFRIPYYVGPLARGNSRFAWMSRK | |
| | SDEKITPWNFDEVVDKEASAQKFIERMTNNDTYLPNEKVLPKHSLLYEKFTV | |
| | YNELTKVKYVTEGMRKPEFLSAEQKQEIFDLLFKKNRKVTKKKLKEDYFKKF | |
| | ECFDSVEISGVEDRFNASLGTYHDLLKIIKDKDFLDNEENEDILEDIVLTLT | |
| | LFEDREMIKERLKKYADLFDDKQLKQLKRRHYTGWGRLSAKLINGIRDKQSG | |
| | KTILDYLKEDGFANRNFMQLIHDDSLTFKEEIEKAQVSGQGDSLHEHIANLA | |
| | GSPAIKKGILQTVKVVDELVKVMGRHKPENIVIEMARENQTTQKGQRNSRQR | |
| | LKKLEEGIKELGSKILKEHPVENQQLQNDKLYLYYLQNGKDMYTGEELDIDR | |
| | LSQYDIDHIIPQSFIKDDSIDNKVLVSSKKARGKSDDVPSEEVVKKMKNYWR | |
| | QLLEAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-133 | MDKKYSIGLDIGTNSVGWAVITDEYKVPSKKFKVLGNTDRHSIKKNLIGALL | 93 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNPENSDVDKLFIQLVQTYNQLFEENPINEEGVDA | |
| | KAILTAKLSKSRRLENLIAQFPGEKRNGLFGNLIALSLGLTPNFKSNFDLAE | |
| | DAKLQLSKDTYDEDLENLLGQIGDQYADLFLAAKNLSDAILLSGILTVNSES | |
| | TKAPLSASMIKRYDEHHQDLTLLKAFVRQQLPEKYKEIFFDESKNGYAGYID | |
| | GGASQEEFYKYIKPILEKLDGTEEFLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKEASAQAFIERMTNFDKNLPTEKVLPKHSLLYEYFTV | |
| | YNELTKVKYVTEGMGKPEFFDGEQKQEIFDGLFKKNRKVTVKQLKDFLFKEF | |
| | EEFRIVDISGVEDAFNASLGTYHDLLKIIKDKDFLDNEENEDILEDIVLTLT | |
| | LFEDREMIEKRLEKYADLFDKKVLKQLKRRRYTGWGRLSKKLINGIRDKQSG | |
| | KTILDFLKSDGFANRNFMQLIHDDSLTFKEEIKKAQVEGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-134 | MDKKYSIGLDIGTNSVGWAVITDEYKVPSKKFKVLGNTDRHSIKKNLIGALL | 94 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNPENSDVQKLFIQLVQTYNQLFEENPINEETVDA | |
| | KAILSAKLSKSRRLENLIAQFPGEKKNGLFGNLIALSLGLIPNFKSNEDLSE | |
| | DAKLQLSKDTYDEDLDNLLAQIGDQYADLFLAAKNLSDAILLSDILTVNDEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMGKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYANLFDKKVLKQLKRRRYTGWGRLSRKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEDKGYKDIQKDLII | |
| | KLPKYSLFELENGRKRLLASAGELQKGNELALPNKYVTFLYHASHYEKLKGS | |
| | PEDNEQKRLYVEQHRDYFDEILDQISEFSERYILADKNLEKIKSLYNEFEDK | |
| | SIEELAESFINLFTLTALGAPAAFKFFDTTIDRKRYTSTKEILDATLIHQSI | |
| | TGLYETRIDLSQLGGD | |
| CasEnd-135 | MKKKYSIGLDIGTNSVGWAVITDEYKVPSKKFKVLGNTDRHSIKKNLIGALL | 95 |
| | FDSGETAEATRLKRTARRRYTRRKNRIRYLQEIFAEEMAKVDDSFFQRLEES | |
| | FLVEEDKRNERHPIFGNIVDEVAYHEKYPTIYHLRKELVDSDEKADLRLIYL | |
| | ALAHMIKFRGHFLIEGDLNSENSDVQKLFIQLVQTYNQLFEESPLDEEGVDA | |
| | KAILSDKVSKSRRLENLIALFPGEKKNGLFGNLIALSLGLTPNFKTNFVLAE | |
| | DAKLQFSKDTYDEDLENLLGQIGDQYADLFLAAKNLSDAILLSGILRVDDES | |
| | TKAPLSASMIKRYDEHHQDLTLLKTLVRQQLPEKYKEIFFDKTKNGYAGYID | |
| | GGASQEEFYKYIKPILEKMDGTEYLLDKLEREDLLRKQRTFDNGSIPHQIHL | |
| | GELHAILRRQEDFYPFLKENQEKIEKILTFRIPYYVGPLARGNSRFAWMTRK | |
| | SDETITPWNFEEVVDKGASAQAFIERMTNFDKNLPEEKVLPKHSLLYEKFTV | |
| | YNELTKVKYVTEGMGKPEFLSANQKKEIVDLLFKKNRKVTVKKLKEFYFQKF | |
| | DCFRIVEISGVEDRFNASLGTYHDLLKIIKDKEFLDNEANEEILEDIVLTLT | |
| | LFEDREMIEKRLKKYAHLFDKKVMKQLKRRRYTGWGRLSKKLINGIRDKQSG | |
| | KTILDFLISDGFANRNFMQLIHDDSLTFKEEIKKAQVKGQSDSLHEQIADLA | |
| | GSPAIKKGILQTVKVVDELVKVMGRHKPENIVIEMARENQTTQKGQRNSRER | |
| | LKRIEEGIKKLGSKILKEYPVENTQLQNDKLYLYYLQNGRDMYTGEELDIDR | |
| | LSDYDVDHIVPQSFIKDDSIDNKVLTRSKEARGKSDDVPSEEVVRKMKSYWR | |
| | QLLKAGLITQRKFDNLTKAERGGLSELDKAGFIKRQLVETRQITKHVAQILD | |
| | SRMNTERDENDKLIRDVKVITLKSKLVSDFRKDFQFYKVREINDYHHAHDAY | |
| | LNAVVGTALIKKYPKLESEFVYGDYKVYDSYKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-136 | MDKKYSIGLDIGTNSVGWAVITDEYKVPSKKFKVLGNTDRHSIKKNLIGALL | 96 |
| | FDSGETAEATRLKRTARRRYTRRKNRICYLQEIFSNEMAKVDDSFFHRLEES | |
| | FLVEEDKRHERHPIFGNIVDEVAYHEKYPTIYHLRKKLADSTEKADLRLIYL | |
| | ALAHIIKFRGHFLIEGDLNSDNSDVQKLFIQLVQTYNQLFEENPLNESGVDA | |
| | KAILSAKLSKSRRLENLIAQLPGEKKNGLFGNLIALSLGLTPNFKSNEGLSE | |
| | DAKLQLSKDTYDEDLENLLAQIGDQYADLFLAAKNLSDAILLSDILTVNDEI | |
| | TKAPLSASMIKRYDEHHQDLTLLKALVRQQLPEKYKEIFFDKSKNGYAGYID | |
| | GGASQEEFYKYIKPILEKMDGAEELLAKINREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKEASAQAFIERMTNFDKNLPSEKVLPKHSLLYEYFTV | |
| | YNELTKVKYVTEGMGKAEFFDANQKQEIFDGLFKKYRKVTKKRLLEFLDKEF | |
| | DEFRIVDISGVEDAFNASLGTYHDLLKIIKDKDFLDNEENEDILEDIVLTLT | |
| | LFEDREMIEERLEKYANLFDKKVLKQLKRRRYTGWGRLSKKLINGIRDKQSG | |
| | KTILDFLKSDGFANRNFMQLIHDDSLTFKEEIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-137 | MDKKYSIGLDIGTNSVGWAVITDEYKVPSKKFKVLGNTDRKSIKKNLIGALL | 97 |
| | FDSGETAEATRLKRTARRRYTRRKNRIRYLQEIFAEEMAKVDDSFFHRLEES | |
| | FLVEEDKRNERHPIFGNIVDEVAYHEKYPTIYHLRKKLVDSDEKADLRLIYL | |
| | ALAHMIKFRGHFLIEGDLNSDNSDVQKLFKELVQTYNQLFEEKPIDASGVDA | |
| | KAILSEKLSKSRRLENLIAELPGEKKNGLFGNLIALSLGLTPNFKSNFDLEE | |
| | DAKLQLSKDTYDDDLENLLGQIGDQYADLFLAAKNLSDAILLSGILRVNTEI | |
| | TKAPLSASMIKRYDEHHQDLTLLKQLVRQQLPEKYKEIFFDQSKNGYAGYID | |
| | GGASQEEFYKYIKKILEKMDGTEELLDKLNREDLLRKQRTFDNGSIPHQIHL | |
| | GELHAILRRQEEYYPFLKENREKIEKILTFRIPYYVGPLARGNSRFAWMKRK | |
| | SDETITPWNFEEVVDKGASAQAFIERMTNFDKNLPNEKVLPKHSLLYEYFTV | |
| | YNELTKVKYVTEGMRKPEFLSGEQKKEIVDLLFKKNRKVTVKQLKEDYFKKE | |
| | ECFDSVEISGVEDRFNASLGTYHDLLKIIKDKDFLDNDENEDILEDIVLTLT | |
| | LFEDREMIKERLEKYANLFDDKVMKQLKRRRYTGWGRLSKKLINGIRDKQSG | |
| | KTILDFLIADGFANRNFMQLIHDDSLTFKDEIEKAQVIGKGESLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTEYDENDKLIRDVKVITLKSKLVSDFRKDFGFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNEETGEIVWDKEKDFATVRKVLSMPQV | |
| | NIVKKTEVQTGGFSKESILPKRNSDKLIARKKDWDPTKYGGFDSPTVAYSVL | |
| | VVAKIEKGKAKKLKTVKELLGITIMERSAFEKNPVAFLEDKGYQEVKEDLII | |
| | KLPKYSLFELENGRKRMLASAGELQKGNELALPSKYVNFLYLASHYEKLKGS | |
| | TEDNKYKQLYVEQHREYLDEIIDQISEFSERVILADANLEKVKSAYEKHREK | |
| | SIEEQAENIIHLFTLTALGAPAAFKYFDTTIDRKRYTSTKEVLNATLIHQSI | |
| | TGLYETRIDLSQLGGD | |
| CasEnd-138 | MDKKYSIGLDIGTNSVGWAVITDEYKVPSKKFKVLGNTDRHSIKKNLIGALL | 98 |
| | FDSGETAEATRLKRTARRRYTRRKNRICYLQEIFSNEMAKVDDSFFQRLEES | |
| | FLVEEDKKNERHPIFGNIVDEVAYHEKYPTIYHLRKKLVDSDEKADLRLIYL | |
| | ALAHMIKFRGHFLIEGDLNSDNSDVQKLFIELVDTYNQLFEESPIEAEEVDA | |
| | KAILSERLSKSRRLENLIAELPGEKKNGLFGNLIALSLGLTPNFKSNFDLSE | |
| | DAKLQLSKDTYDDDLEELLGQIGDEYADLFLAAKNLSDAILLSGILRVKTES | |
| | TKAPLSASMIKRYDEHHQDLTLLKQLVRKQLPEKYKEIFFDQSKNGYAGYID | |
| | GGASQEEFYKYIKKILEKMDGTEELLDKLDREDLLRKQRTFDNGSIPHQIHL | |
| | GELRAILRRQEKYYPFLKENREKIEKILTFRIPYYVGPLARGNSRFAWMIRK | |
| | SDETITPWNFDEVVDKGASAEKFIERMTNFDKNLPEEKVLPKHSLLYEYFTV | |
| | YNELTKVKYVTEGMRKPEFLDGEQKKEIVDLLFKKNRKVTVKQLKEYYFKEF | |
| | ECFDSVEISGVEDRFNASLGTYHDLLKIIKDKAFLDNEENEKILEDIVLTLT | |
| | LFEDREMIKKRLEKYANLFDKKVMKQLKRRRYTGWGRLSKKLINGIRDKQSG | |
| | KTILDFLIDDGFTNRNFMQLIHDDSLTFKDEIEKAQVIGKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-139 | MDKKYSIGLDIGTNSVGWAVITDEYKVPSKKFKVLGNTDRHSIKKNLIGALL | 99 |
| | FDSGETAEATRLKRTARRRYTRRKNRICYLQEIFSNEMAKVDDSFFHRLEES | |
| | FLVEEDKRHERHPIFGNIVDEVAYHEKYPTIYHLRKKLADSTEKADLRLIYL | |
| | ALAHIIKFRGHFLIEGDLNSDNSDVQKLFIQLVQTYNQLFEENPINESGVDA | |
| | KAILSAKLSKSRRLENLIAQFPGEKKNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRKQLPEKYKEIFFDQSKNGYAGYID | |
| | GGASQEDFYKFIKPILEKMDGTEELLAKINREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKEASAQSFIERMTNFDKNLPEEKVLPKHSLLYEYFTV | |
| | YNELTKVKYVTEGMRKPEFLSGEQKKEIVDLLFKKNRKVTVKQLKEFYFKKI | |
| | ECFDSVEISGVEDRFNASLGTYHDLLKIIKDKEFLDNEENEDILEDIVLTLT | |
| | LFEDKEMIEERLKKYADLFDDKVLKKLKRRRYTGWGRLSRKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRIEEGLKELGSQILKEHPVENTQLQNEKLYLYYLQNGKDMYTGDELDIDR | |
| | LSDYDVDHIIPQSFIKDDSIDNKVLTSSDKARGKSDNVPSEEVVKKMKNYWR | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKIEKGKAKKLKTVKELVGITIMERSSFEKDPVAFLEDKGYKNIQEDLII | |
| | KLPKYSLFELENGRKRMLASAGELQKGNELALPSKYVILLYLASHYEKLKGS | |
| | PEDNEKKLLYVEQHRHYFDEIFDQISEFSERYILADKNLEKILSLYNENEDK | |
| | SISEQAESFINLFTLTALGAPAAFKFFDTTIDRKRYTSTKEILNATLIHQSI | |
| | TGLYETRIDLSQLGGD | |
| CasEnd-140 | MDKKYSIGLDIGTNSVGWAVITDEYKVPSKKFKVLGNTDRHSIKKNLIGALL | 100 |
| | FDSGETAEATRLKRTARRRYTRRKNRICYLQEIFSNEMAKVDPSFFKRLEES | |
| | FLVEEDKSGSRHPIFGNIVEEVAYHEKYPTIYHLRKKLVDSEEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTKD | |
| | TRAPLSASMIKRYDEHHQDLTELKKLVRKYLPEKYKEIFFDQNKNGYAGYID | |
| | GGATQEEFYEYIKPILESMPGTKHLLEKLENRDLLRKQRTEDNGSIPHQIHL | |
| | GELRAILERQEKFYPFLKENREKIEKILSFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRKSDKLIARKKDWDPKKYGGFDSPTVAYSVL | |
| | VVAKKPVGKKKELKEVKELLGITIMERSKFEENPLKFLEEKGYKDVKMDEII | |
| | KLPKYSLFELGNGRKRMLASAGELQKGNELALPSEYVNFLYLASNYEKLKGK | |
| | PEEIKEKQEYVEKNKEYLDKIIDQISEFSQRVIKADANLKKVLEAYEKHKDK | |
| | PIKEQAENIIHLFTLTRLGAPAAFKYFDETIDRKRYTSTKEVLDATLIHQSI | |
| | TGLYETRIDLSFLGGD | |
| CasEnd-141 | MDKKYSIGLDIGTNSVGWAVITDEYKVPSKKFKVLGNTDRQSIKKNLIGALL | 101 |
| | FDSGETAEATRLKRTARRRYTRRKNRIRYLQEIFAEEMSKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVQKLFIQLVDTYNQLFEENPIGEEGVDA | |
| | KAILSARLSKSRRLENLIAQIPGEKRNGLFGNLIALSLGLTPNFKSNFDLAE | |
| | DAKLQLSKDTYDEDLDNLLAQIGDQYADLFLAAKNLSDAILLSGILTVNDEI | |
| | TKAPLSASMIKRYDEHHQDLTLLKDLVRQQLPEKYKEIFFDDSKNGYAGYID | |
| | GGASQEEFYKYIKPILEKLDGAEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQSFIERMTNFDKNLPNEKVLPKHSLLYEYFTV | |
| | YNELTKVKYVTEGMRKPEFLSSEQKEAIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDKKVLKQLKRRRYTGWGRLSRKLINGIRDKQSG | |
| | KTILDFLKDDGFANRNFMQLIHDDSLTFKEEIQKAQVIGQGDSLHEQIANLA | |
| | GSPAIKKGILQTLKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENNKLIREVKIITLKSKLVSDFRKDFQFYKVREINDYHHAHDAY | |
| | LNAVVGTALIKKYPKLESEFVYGDYKVYDVRKMIRKSEQEIGKATAKYFFYS | |
| | NIMNFFKSEITLANGEIIKRPLIETNEETGEIVWDKGKDFATVRKVLSMPQV | |
| | NIVKKTEVQTGGFSKESILPKRNSDKLIARKKDWDPKKYGGFDSPTVAYSVL | |
| | VIAKVEKGKTKKLKTVKELVGITIMERSSFEKNPIAFLEKKGYQEVQKHLII | |
| | KLPKYSLFELENGRKRLLASAGELQKGNELALPSKYVTLLYLASNYEKLKGK | |
| | SEDNEKKKEYVEQHREEFDEIFNQIIEFSERYILADKNLSKIKELENKNEDS | |
| | DITELAENFIHLFTFTSLGAPAAFKFFDKTIDRKRYTSTKECLDATLIHQSI | |
| | TGLYETRIDLSQLGGD | |
| CasEnd-142 | MDKKYSIGLDIGTNSVGWAVITDEYKVPSKKFKVLGNTDRHSIKKNLIGALL | 102 |
| | FDSGETAEATRLKRTARRRYTRRKNRIRYLQEIFSSEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPDNSDVDKLFIQLVQTYNQLFEENPINESGVDA | |
| | KAILSARLSKSRRLENLIAQLPGQKKNGLFGNLIALSLGLTPNFKSNEDLAE | |
| | DAKLQLSKDTYDEDLDNLLAQIGDQYADLFLAAKNLSDAILLSDILTVNDEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSAEQKEAIVDLLFKKNRKVTVKQLKDDYFKKI | |
| | ECFDSVEISGVEDRFNASLGTYHDLLKIIKDKDFLDNEENEDILEDIVLTLT | |
| | LFEDREMIEERLKKYAHLFDKKVLKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQSVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTERDENDKLIREVKIITLKSKLVSDFRKDFQFYKVREINDYHHAHDAY | |
| | LNAVVGTALIKKYPKLESEFVYGDYKVYDVRKMIAKSEQEIGKATAKYFFYS | |
| | NIMNFFKTEITLANGEIRKRPVIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKSKKLKSVKELVGITIMERSSFEKDPVAFLEAKGYKDVQKDLII | |
| | KLPKYSLFELENGRKRMLASAGELQKGNELALPSKYVTFLYLASHYEKLKGK | |
| | PEDNEQKQEYVEQHRHYFDEIFEQIIEFSERYILADKNLDKILSLYSKERDK | |
| | SIREQAENFIHLFTLTSLGAPAAFKFFDTTIDRKRYTSTKEILDATLIHQSI | |
| | TGLYETRIDLSQLGGD | |
| CasEnd-143 | MDKKYSIGLDIGTNSVGWAVITDEYKVPSKKFKVLGNTDRHSIKKNLIGALL | 103 |
| | FDSGETAEATRLKRTARRRYTRRKNRICYLQEIFSNEMAKVDDSFFHRLEES | |
| | FLVEEDKRNERHPIFGNIVDEVAYHEKYPTIYHLRKKLADSDEKADLRLVYL | |
| | ALAHMIKFRGHFLIEGDLNSENSDVQKLFIQLVQTYNQLFEENPLNEEGVDA | |
| | EAILSERLSKSRRLENLIAQFPGEKKNGLFGNLIALSLGLTPNFKSNEDLAE | |
| | DAKLQLSKDTYDEDLENLLAQIGDQYADLFLAAKNLSDAILLSDILTVNDES | |
| | TKAPLSASMVKRYDEHHQDLKLLKALVRQQLPEKYKEIFSDKSKNGYAGYID | |
| | GKTSQEEFYKYIKPILEKMDGSEEFLEKINREDFLRKQRTFDNGSIPHQIHL | |
| | QELHAILRRQEKYYPFLKENREKIEKILTFRIPYYVGPLARGNSRFAWMTRK | |
| | SDEKITPWNFDEVVDKEASAEKFIERMTNFDTYLPEEKVLPKHSLLYEKFTV | |
| | YNELTKVKYVTEGMRKPEFFSGEQKQEIFDLLFKKNRKVTVKKLKEDYFKKF | |
| | ECFDIVEISGLEDRFNASLGTYHDLLKIIKDKDFLDNEENEDILEDIVLTLT | |
| | LFEDREMIEKRLKKYADLFDDKVLKQLKRRHYTGWGRLSKKLINGIRDKQSG | |
| | KTILDFLISDGFANRNFMQLIHDDSLTFKEEIEKAQVSGQGDSLHEHIANLA | |
| | GSPAIKKGILQTVKVVDELVKVMGRHKPENIVIEMARENQTTQKGQRNSRER | |
| | LKRIEEAIKELGSKILKEHPVENTQLQNDRLYLYYLQNGKDMYTGEELDIDR | |
| | LSQYDIDHIIPQSFIKDDSIDNKVLVSSAKARGKSDNVPSEEVVKKMKNYWR | |
| | QLLDAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-144 | MDKKYSIGLDIGTNSVGWAVITDEYKVPSKKFKVLGNTDRHSIKKNLIGALL | 104 |
| | FDSGETAEATRLKRTARRRYTRRKNRICYLQEIFSNEMAKVDDSFFHRLEES | |
| | FLVEEDKRNDRHPIFGNIVEEVAYHEKYPTIYHLRKHLADSPEKADLRLVYL | |
| | ALAHIIKFRGHFLIEGDLDAENTDVQKLFKQLVEAYNQTFEESHLEIETVDA | |
| | KSILTEKLSKSRRLENLIAKFPGEKKNGLFGNLIALSLGLTPNFKSNFDLSE | |
| | DAKLQFSKDTYDEDLEELLGQIGDDYADLFDAAKNLYDAILLSGILTVDDNS | |
| | TKAPLSASMVKRYDEHHQDLTLLKEFVREKLPEKYKEIFFDQSKNGYAGYID | |
| | GGASQEDFYKYLKKLLEKIDGSEEFLDKIDREDFLRKQRTFDNGSIPHQIHL | |
| | QELKAIIRRQEEYYPFLKENKEKIEKILTFRIPYYVGPLARGNSRFAWMERK | |
| | SDETITPWNEDDIVDKEKSAEKFIERMTNFDKNLPEEKVLPKHSLLYEKFTV | |
| | YNELTKVKYVTEGGKKPKFFDANQKQEIVDNLFKKYRKVTKKQLLEYLAKEF | |
| | DEFRIVDISGVEDRFNASLGTYHDLKKILGDKSFLDDDKNEEILEDIILTLT | |
| | LFEDREMIKKRLEKYSDLFDKKQIKKLSRRRYTGWGRLSKKLINGIRDKQSG | |
| | KTILDYLIDDGYANRNFMQLIHDDSLSFKEEIEKAQVIGDGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-145 | MDKKYSIGLDIGTNSVGWAVITDEYKVPSKKFKVLGNTDRHSIKKNLIGALL | 105 |
| | FDSGETAEATRLKRTARRRYTRRKNRIRYLQEIFSEEMAKVDDSFFHRLEES | |
| | FLVEEDKKNERHPIFGNIVDEVAYHEKYPTIYHLRKKLVDSDEKADLRLIYL | |
| | ALAHMIKFRGHFLIEGDLNSDNSDVQKLFEQLVQTYNQLFEENPIEAEGVDA | |
| | KAILSEKLSKSRRLENLIAQLPGEKKNGLFGNLIALSLGLTPNFKSNFDLAE | |
| | DAKLQLSKDTYDDDLENLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDLLRKQRTFDNGSIPHQIHL | |
| | GELHAILRRQEKYYPFLKENREKIEKILTFRIPYYVGPLARGNSRFAWMTRK | |
| | SDETITPWNFEEVVDKGASAQKFIERMTNFDKNLPNEKVLPKHSLLYEYFTV | |
| | YNELTKVKYVTEGMRKPEFLDGEQKKEIVDLLFKKNRKVTVKQLKEDYFKKF | |
| | ECFDSVEISGVEDRFNASLGTYHDLLKIIKDKDFLDNEENEDILEDIVLTLT | |
| | LFEDREMIKERLEKYANLFDKKVMKQLKRRRYTGWGRLSKKLINGIRDKQSG | |
| | KTILDFLKADGFANRNFMQLIHDDSLTFKDEIEKAQVIGKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTEYDENDKLIRDVKVITLKSKLVSDFRKDFGFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELLGITIMERSSFEKNPVAFLEAKGYQEVKKDLII | |
| | KLPKYSLFELENGRKRMLASAGELQKGNELALPSKYVNFLYLASHYEKLKGS | |
| | PEDNEQKQLFVEQHKEYLDEIIEQISEFSKRVILADANLEKVKSAYEKHEDK | |
| | SIEEQAENIIHLFTLTNLGAPAAFKYFDTTIDRKRYTSTKEVLNATLIHQSI | |
| | TGLYETRIDLSQLGGD | |
| CasEnd-146 | MDKKYSIGLDIGTNSVGWAVITDEYKVPSKKFKVLGNTDRHSIKKNLIGALL | 106 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPENSDVDKLFIQLVQTYNQLFEENPINESGVDA | |
| | KAILTAKLSKSRRLENLIAQLPGEKKNGLFGNLIALSLGLTPNFKSNEDLAE | |
| | DAKLQLSKDTYDEDLDNLLAQIGDQYADLFLAAKNLSDAILLSDILTVNDEI | |
| | TKAPLSASMIKRYDEHHQDLTLLKALVRQQLPEKYKEIFFDPSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKEEIVDLLFKTNRKVTVKQLKEDYFKKI | |
| | ECFDSVEISGVEDRFNASLGTYHDLLKIIKDKDELDNEENEDILEDIVLTLT | |
| | LFEDREMIEERLEKYAHLFDKKVLKQLKRRRYTGWGRLSRKLINGIRDKQSG | |
| | KTILDFLKSDGFANRNFMQLIHDDSLTFKEEIQKAQVIGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTERDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGTIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEAKGYKEVQKDLII | |
| | KLPKYSLFELENGRKRLLASAGELQKGNELALPGKYVILLYLASNYEKLKGK | |
| | PEDNEQKLEYVEQHRHYFDEIVDQISEFSERYILADANLSKILSLYNEHRDK | |
| | PIREQAENFIHLFTFTSLGAPAAFKFFDTTIDRKRYTSTSEILNATLIHQSI | |
| | TGLYETRIDLSQLGGD | |
| CasEnd-147 | MDKKYSIGLDIGTNSVGWAVITDEYKVPSKKFKVLGNTDRHSIKKNLIGALL | 107 |
| | FDSGETAEATRLKRTARRRYTRRKNRICYLQEIFSNEMAKVDPSFFQRLEES | |
| | FLVEEDKTGSRHPIFGNIVEEVAYHEKYPTIYHLRKKLVDSDEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVDIND | |
| | TRAPLSASMIKRYDDHHQDLTKLKELVRKYLPEKYKEIFFDQNSNGYAGYID | |
| | GGATQEEFYKYIKPILESMPGTKDLLKKLENKDLLRKQRTFDNGSIPHQIHL | |
| | GELRAILERQEKFYPFLKENREKIEKILSFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKKEKGKKKKLVKVKELLGITIMERSKFEKDPLGFLESKGYKDVKEDEII | |
| | KLPKYSLFELGNGRKRMLASAGELQKGNELALPSEYVNFLYLASNYEKLKGD | |
| | PKKQEEKQKYVEKNKEYLDKIIEQISEFSRRVIKADANLEKVLKAYEKHKDK | |
| | PISEQAENIIHLFTLTALGAPAAFKYFDEVIDRKRYTSTKEVLDATLIHQSI | |
| | TGLYETRIDLSFLGGD | |
| CasEnd-148 | MDKKYSIGLDIGTNSVGWAVITDEYKVPSKKFKVLGNTDRHSIKKNLIGALL | 108 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVQKLFIQLVQTYNQLFEENPINEEGVDA | |
| | KAILSAKLSKSRRLENLIAQLPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKYIKPILEKMDGTEYLLAKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMGKPEFLSAEQKQEIVDLLFKKNRKVTVKQLKEFLFKEI | |
| | ECFDSVEISGVEDAFNASLGTYHDLLKIIKDKDELDNEENEDILEDIILTLT | |
| | LFEDREMIEERLKKYANLFDKKVLKQLKRRRYTGWGRLSKKLINGIRDKQSG | |
| | KTILDFLKSDGFANRNFMQLIHDDSLTFKEEIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPIAFLEAKGYKNIQEDLII | |
| | KLPKYSLFELENGRKRLLASAGELQKGNELALPQKYVTFLYLASHYEKLKGK | |
| | PEDEEQKQLYVEQHRHYFDEILEQISEFSERYILADKNLEKIKELYNKFEDY | |
| | SISELAESFINLFTLTALGAPAAFKFFDTTIDRKRYTSTKEILNATLIHQSI | |
| | TGLYETRIDLSQLGGD | |
| CasEnd-149 | MDKSYSIGLDIGTNSVGWAVITDEYKVPSKKFKVLGNTDRHSIKKNLLGALL | 109 |
| | FDSGETAEATRLKRTARRRYTRRKNRIRYLQEIFAEEMAKVDDSFFHRLEES | |
| | FLVEEDKRHERHPIFGNIVDEVAYHENYPTIYHLRKHLADSTEKADLRLIYL | |
| | ALAHIIKFRGHFLIEGDLNAENTDVQKLFIQLVQTYNQLFEESPINEEGVDA | |
| | KAILTAKLSKSRRLENLIKQIPGEKKNGLFGNLIALSLGLTPNFKSNEDLAE | |
| | DAKLQLSKDTYDEDLDNLLAQIGDQYADLFLAAKNLSDAILLSDILTVNTEI | |
| | TKAPLSASMIKRYDEHHQDLTLLKKLVREQLPEKYKEIFFDESKNGYAGYID | |
| | GGASQEDFYKYIKPILEKLDGTEEFLAKINREDFLRKQRTFDNGSIPHQIHL | |
| | GELHAILRRQEDFYPFLKENKEKIEKILTFRIPYYVGPLARGNSRFAWLTRK | |
| | SDETITPWNFEEVVDKEASAQAFIERMTNFDKNLPEEKVLPKHSLLYEYFTV | |
| | YNELTKVKYITEGMRKPAFLSSEQKKEIVDLLFKKNRKVTVKQLKEFYFKKI | |
| | ECFDSVEISGVEDRFNASLGTYHDLLKIIKDKDFLDNEENEDILEDIVLTLT | |
| | LFEDREMIEKRLSKYADLFDKKVLKQLKRRRYTGWGRLSAKLINGIRDKQSG | |
| | KTILDFLKDDGFANRNFMQLIHDDSLTFKEEIQKAQVIGQGDSLHEQIANLA | |
| | GSPAIKKGILQSIKIVDELVKVMGRHKPENIVIEMARENQTTQKGQKNSRER | |
| | LKRLEEGIKKLGSQILKEHPVENTQLQNDKLYLYYLQNGKDMYTGEELDIDR | |
| | LSDYDVDHIIPQSFIKDDSIDNKVLTSSAKARGKSDNVPSEEVVKKMKSYWR | |
| | QLLNAKLISQRKFDNLTKAERGGLSELDKAGFIKRQLVETRQITKHVAQILD | |
| | SRFNTKYDENDKLIREVKIITLKSKLVSDFRKDFGFYKVREINDYHHAHDAY | |
| | LNAVVGTALIKKYPKLESEFVYGDYKVYDSRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVADVEKGKSKKLKTVKELVGITIMERSSFEKNPIAFLEAKGYKNIQKDLII | |
| | KLPKYSLFELENGRKRLLASAGELQKGNELALPQKYVTLLYHAKHYEKLKES | |
| | PEDNEKHLEYVEQHRHEFDEIFDQISEFSERYILADKNLEKIKELYNKNEDK | |
| | DISELAESFINLFTFTALGAPAAFKFFDTTIDRKRYTSTTEILNATLIHQSI | |
| | TGLYETRIDLSQLGGD | |
| CasEnd-150 | MKKKYSIGLDIGTNSVGWAVITDEYKVPSKKFKVLGNTDRQSIKKNLLGALL | 110 |
| | FDSGETAEATRLKRTARRRYTRRKNRIRYLQEIFAEEMAKVDDSFFHRLEES | |
| | FLVEEDKRHERHPIFGNIVDEVAYHENYPTIYHLRKKLADSTEKADLRLIYL | |
| | ALAHIIKFRGHFLIEGDLNPENTDVQKLFIQLVQTYNQLFEENPLSEEGVDA | |
| | KAILTAKLSKSRRLENLIAQIPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQTKNGYAGYID | |
| | GGASQEEFYKFIKPILEKLDGSEELLAKINREDFLRKQRTFDNGSIPHQIHL | |
| | GELHAILRRQEDFYPFLKENREKIEKILTFRIPYYVGPLARGNSRFAWLTRK | |
| | SDETITPWNFEEVVDKEASAQAFIERMTNFDKNLPNEKVLPKHSLLYEYFTV | |
| | YNELTKVKYVTEGMRKPEFLSAEQKEEIVDLLFKTNRKVTVKQLKEDLFKKI | |
| | ECFDSVEISGVEDRFNASLGTYHDLLKIIKDKDFLDNEENEDILEDIVLTLT | |
| | LFEDREMIENRLEKYADLFDKKVLKQLKRRRYTGWGRLSRKLINGIRDKQSG | |
| | KTILDFLKDDGFANRNFMQLIHDDSLTFKEEIKKAQVIGQGDSLHEQIANLA | |
| | GSPAIKKGILQSVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRENTERDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKIEKGKAKKLKTVKELVGITIMERSSFEKDPVAFLEAKGYKNIQKDLII | |
| | KLPKYSLFELENGRKRLLASAGELQKGNELALPQKYVTLLYLASHYEKLKGK | |
| | PEDLEKKLEYVEQHRDEFDEIFEQISEFSERYILADKNLEKIKELYKEFRDK | |
| | SIEELAENFIHLFTFTALGAPAAFKFFDKTIDRKRYTSTTEILNATLIHQSI | |
| | TGLYETRIDLSKLGGD | |
| CasEnd-151 | MDKKYSIGLDIGTNSVGWAVITDEYKVPSKKFKVLGNTDRQSIKKNLIGALL | 111 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNSDNSDVDKLFIQLVQTYNQLFEENPINESGVDA | |
| | KAILSARLSKSRRLENLIAQLPGEKKNGLFGNLIALSLGLTPNFKSNFDLAE | |
| | DAKLQLSKDTYDDDLENLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKKAIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVLKQLKRRRYTGWGRLSRKLINGIRDKQSG | |
| | KTILDFLKSDGFANRNFMQLIHDDSLTFKEAIQKAQVSGQGDSLHEQIANLA | |
| | GSPAIKKGILQTVKIVDELVKVMGRHKPENIVIEMARENQTTQKGQKNSRER | |
| | MKRIEEGLKELGSQILKEHPVENTQLQNEKLYLYYLQNGKDMYTGQELDIDR | |
| | LSDYDVDHIIPQSFIKDDSIDNKVLTSSKEARGKSDNVPSEEVVKKMKNYWR | |
| | QLLKAKLISQRKFDNLTKAERGGLTELDKAGFIKRQLVETRQITKHVAQILD | |
| | SRMNTEYDENDKLIRKVKIITLKSKLVSDFRKDFQFYKVREINNYHHAHDAY | |
| | LNAVVGTALIKKYPKLESEFVYGDYKVYDVRKMIAKSEQEIGKATAKRFFYS | |
| | NIMNFFKTEIKLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELVGITIMERSSFEKDPVDFLEAKGYKEIQKDLII | |
| | KLPKYSLFELENGRKRMLASAGELQKGNELALPSKYVKFLYLASHYEKLKGS | |
| | PEDNEQKQLYVEQHKHYFDEIVDQISEFSERYILADANLDKILSAYNKHRDK | |
| | SIREQAENIINLETLTNLGAPAAFKFFDTTIDRKRYTSTKEILDATLIHQSI | |
| | TGLYETRIDLSQLGGD | |
| CasEnd-152 | MDKKYSIGLDIGTNSVGWAVITDEYKVPSKKFKVLGNTDRHSIKKNLIGALL | 112 |
| | FDSGETAEATRLKRTARRRYTRRKNRIRYLQEIFSEEMAKVDDSFFHRLEES | |
| | FLVEEDKRNERHPIFGNIVDEVAYHEKYPTIYHLRKKLADSDEKADLRLIYL | |
| | ALAHMIKFRGHFLIEGDLNSDNSDVDKLFIQLVQTYNQLFEENPINEEGVDA | |
| | KAILSEKLSKSRRLENLIAQFPGEKKNGLFGNLIALSLGLTPNFKSNEDLAE | |
| | DAKLQLSKDTYDDDLENLLAQIGDQYADLFLAAKNLSDAILLSDILTVNDEI | |
| | TKAPLSASMVKRYDEHHQDLTLLKKLVRQQLPEKYKEIFFDQSKNGYAGYID | |
| | GGASQEEFYKYLKPILEKMDGTEELLDKIEREDFLRKQRTFDNGSIPHQIHL | |
| | GELHAILRRQEKYYPFLKDNREKIEKILTFRIPYYVGPLARGNSRFAWMTRK | |
| | SDETITPWNFEEVVDKGASAQSFIERMTNFDKNLPNEKVLPKHSLLYEKFTV | |
| | YNELTKVKYVTEGMRKPAFLSAEQKQEIVDLLFKKNRKVTVKQLKEDYFKKF | |
| | ECFDSVEISGVEDRFNASLGTYHDLLKIIKDKDFLDNEENEDILEDIVLTLT | |
| | LFEDREMIEKRLKKYANLFDDKVLKKLKRRRYTGWGRLSKKLINGIRDKQSG | |
| | KTILDFLKSDGFANRNFMQLIHDDSLTFKEEIQKAQVSGQGESLHELIANLA | |
| | GSPAIKKGILQTLKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDPKKYGGFDSPTVAYSVL | |
| | VIAKVEKGKAKKLKTVKELVGITIMERSSFEKNPIAFLEDKGYKEVKKDLII | |
| | KLPKYSLFELENGRKRLLASAGELQKGNELALPSKYVTLLYLASHYEKLKGS | |
| | PEDNEQKQLFVEQHKHYFDEILDQISEFSKRVILADANLEKIKSLYDKNRDA | |
| | SIEEQAENFIHLFTFTNLGAPAAFKFFDTTIDRKRYTSTKEILNATLIHQSI | |
| | TGLYETRIDLSQLGGD | |
| CasEnd-153 | MDKKYSIGLDIGTNSVGWAVITDEYKVPSKKFKVLGNTDRHSIKKNLIGALL | 113 |
| | FDSGETAEATRLKRTARRRYTRRKNRICYLQEIFSNEMAKVDENFFQRLEES | |
| | FLVEEDKRGERHPIFGNIVEEVAYHEKYPTIYHLRKHLADSDEKADLRLVYL | |
| | ALAHIIKFRGHFLIEGDLDAENTDVQKLFKQLVETYDRTFEESPLEEFTVDA | |
| | ESILTEKLSKSRRLENLIAQFPGEKKNGLFGNFIALSLGLTPNFKSNEDLSE | |
| | DAKLQFSKDTYDEDLEELLGQIGDDYADLFLAAKNLYDAILLSGILTVDDNS | |
| | TKAPLSASMVKRYDEHHQDLTELKAFIRKQLPEKYKEIFFDQSKNGYAGYID | |
| | GGASQEEFYKYLKKILEKIDGSEEFLDKIEREDFLRKQRTFDNGSIPHQIHL | |
| | QELHAIIRRQEEYYPFLKENQEKIEKILTFRIPYYVGPLARGNSRFAWLSRK | |
| | SDETITPWNFDDVVDKEKSAEDFIERMTNFDKNLPEEKVLPKHSLLYEKFTV | |
| | YNELTKVKYVTEGGRKPKFFDANMKQEIFDELFKKYRKVTKKQLLDYLVKEF | |
| | EEFRIVDISGVEDRFNASLGTYHDLKKILGDKDFLDNDENEEILEDIVLTLT | |
| | LFEDREMIKKRLEKYSDLFDKKQLKKLCRRRYTGWGRLSAKLINGIRDKETG | |
| | KTILDYLIDDGEANRNFMQLIHDDNLSFKEEIEKAQVIGDEDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-154 | MDKKYSIGLDIGTNSVGWAVITDEYKVPSKKFKVLGNTDRHSIKKNLIGALL | 114 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVQKLFKQLVQTYNQLFEEKPLDEETVDA | |
| | KAILSARLSKSRRLENLIAQFPGEKKNGLFGNLIALSLGLTPNFKSNFDLAE | |
| | DAKLQLSKDTYDEDLENLLGQIGDQYADLFVAAKNLSDAILLSGILTVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VIAKVEKGKAKKLKTVKELVGITIMERSAFEKDPVAFLEAKGYKNIQKDLII | |
| | KLPKYSLFELENGRKRLLASAGELQKGNELALPQKYVTLLYHAKNYEKLKGS | |
| | PEDEKEKLLYIEEHREEFDEIFDQISEFSKRYILADANLEKIKELYEQNKDA | |
| | SIEELASSFINLLTFTALGAPAAFKFFDTTIDRKRYTSTKEILNATLIHQSI | |
| | TGLYETRIDLSQLGGD | |
| CasEnd-155 | MKKKYSIGLDIGTNSVGWAVITDEYKVPSKKFKVLGNTDRQSIKKNLLGALL | 115 |
| | FDSGETAEATRLKRTARRRYTRRKNRIRYLQEIFAEEMAKVDDSFFQRLEES | |
| | FLVEEDKRHERHPIFGNIVDEVAYHEKYPTIYHLRKELADSTEKADLRLIYL | |
| | ALAHMIKFRGHFLIEGDLNSENSDVDKLFIQLVQTYNQLFEENPLNEETVDA | |
| | KAILSAKLSKSRRLENLIAQFPNQKKNGLFGNLIALSLGLTPNFKSNFDLSE | |
| | DAKLQLSKDTYDEDLDELLGQIGDQYADLFLAAKNLSDAILLSGILTVNDEI | |
| | TKAPLSASMIKRYDEHHQDLTLLKALVREQLPEKYKEIFFDKSKNGYAGYID | |
| | GGASQEEFYKYIKPILEKIDGTEYFLDKINREDFLRKQRTEDNGSIPHQIHL | |
| | GELHAILRRQEDFYPFLKENREKIEKILTFRIPYYVGPLARGNSRFAWMSRK | |
| | SDETITPWNFEEVVDKEASAQSFIERMTNFDKNLPEEKVLPKHSLLYEYFTV | |
| | YNELTKVKYVTEGMGKPEFFSGEQKEEIVDLLFKKNRKVTVKQLKEDLFKEI | |
| | ECFDIVEISGVEDRFNASLGTYHDLLKIIKDKDFLDNEENEDILEDIVLTLT | |
| | LFEDREMIEERLKKYAHLFDKKVLKKLKRRRYTGWGRLSKKLINGIRDKQSG | |
| | KTILDFLKDDGFANRNFMQLIHDDSLTFKEEIQKAQVIGQGDSLHEVIANLA | |
| | GSPAIKKGILQSVKIVDELVKVMGRHKPENIVVEMARENQTTQKGQKNSRER | |
| | LKRLEEGIKELGSQILKEHPVENTQLQNDKLYLYYLQNGKDMYTGEELDIDR | |
| | LSDYDVDHIIPQSFIKDDSIDNKVLTSSAKARGKSDNVPSEEVVKKMKSYWR | |
| | QLLKAKLISQRKFDNLTKAERGGLSELDKAGFIKRQLVETRQITKHVAQILD | |
| | SRFNTKFDENDKLIRDVKIITLKSKLVSDFRKDFQFYKVREINDYHHAHDAY | |
| | LNAVVGTALIKKYPKLESEFVYGDYPVYDVRKMIAKSEQEIGKATAKRFFYS | |
| | NIMNFFKTEIKLADGEIRKRPQIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKNPIAFLEAKGYKDIQKDLII | |
| | KLPKYSLFELENGRKRMLASAGELQKGNELALPAKYVTLLYLASHYEKLKGS | |
| | PEDNEKKMLFVEQHREYFDEILDQISEFSKRYILADKNLSKILELYNENNDK | |
| | DISEQAESFINLFTFTALGAPAAFKFFDTTIDRKRYTSTKEILDSTLIHQSI | |
| | TGLYETRIDLSQLGGD | |
| CasEnd-156 | MKKSYSIGLDIGTNSVGWAVITDDYKVPSKKMKVLGNTDRQSIKKNLLGALL | 116 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEQVADLA | |
| | GSPAIKKGILQSIKIVDEIVKVMGRHAPENIVIEMARENQTTAKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLTDDDKAGFIKRQLVETRQITKHVAQILD | |
| | SRFNTERDDNNKLIRDVKIITLKSKLVSDFRKDFQLYKVREINDYHHAHDAY | |
| | LNAVVGKALIKKYPKLEPEFVYGDYPKYNSYKLVGKSDKERGKATAKMFFYS | |
| | NIMNFFKSDVKLADGTIIKRPVIEVNEETGEIVWNKEKHIATIKKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-157 | MDKKYSIGLDIGTNSVGWAVITDEYKVPSKKFKVLGNTDRKSIKKNLIGALL | 117 |
| | FDSGETAEATRLKRTARRRYTRRKNRIRYLQEIFSEEMAKVDDSFFHRLEES | |
| | FLVEEDKKNERHPIFGNIVDEVAYHEKYPTIYHLRKKLADSTEKADLRLIYL | |
| | ALAHMIKFRGHFLIEGDLNAENSDVQKLFIQLVQTYNQLFEESPLEAEGVDA | |
| | KAILTARLSKSRRLENLIAQLPGEKKNGLFGNLIALSLGLTPNFKSNEDLAE | |
| | DAKLQLSKDTYDEDLENLLAQIGDEYADLFLAAKNLSDAILLSGILTVKDEI | |
| | TKAPLSASMIKRYDEHHQDLTLLKQFVRQQLPEKYKEIFFDQSKNGYAGYID | |
| | GGASQEEFYKYLKKILEKLDGTEEFLDKIEREDFLRKQRTFDNGSIPHQIHL | |
| | QELHAILRRQEDYYPFLKENREKIEKILTFRIPYYVGPLARGNSRFAWMSRK | |
| | SDETITPWNFEEVVDKGASAQKFIERMTNFDKNLPNEKVLPKHSLLYEYFTV | |
| | YNELTKVKYVTEGMRKPIFLSSEQKQEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYADLFDDKVMKQLKRRRYTGWGRLSKKLINGIRDKQSG | |
| | KTILDYLIDDGFANRNFMQLIHDDSLTFKEEIEKAQVSGQGDSLHEVIANLA | |
| | GSPAIKKGILQTIKIVDELVKVMGRHAPENIVIEMARENQTTQKGQKNSRER | |
| | MKRLEEGIKELGSNILKEHPVENTQLQNDKLYLYYLQNGKDMYTGDELDIDR | |
| | LSDYDVDHIVPQSFLKDDSIDNKVLTSSAKARGKSDNVPSEEVVKKMKNYWK | |
| | QLLDAKLITQRKFDNLTKAERGGLTELDKAGFIKRQLVETRQITKHVAQILD | |
| | SRFNTKYDENDKLIRDVKIITLKSKLVSDFRKDFQLYKVREINNYHHAHDAY | |
| | LNAVVGTALIKKYPKLESEFVYGDYKVYDVRKMIAKSEQEIGKATAKMFFYS | |
| | NIMNFFKTEVTLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VIAKVEKGKAKKLKTVKELVGITIMERSSFEKNPIDFLEAKGYKEVKKDLII | |
| | KLPKYSLFELENGRKRLLASAGELQKGNELALPNKYVELLYLAKHYEKLKGS | |
| | PEDNEQKQLFVEQHKEYFDEILEQISEFSKRVILADANLEKIKKLYEKNEDK | |
| | SIEEQAENFINLLTFTALGAPAAFKFFDTTIDRKRYTSTKEILNATLIHQSI | |
| | TGLYETRIDLSQLGGD | |
| CasEnd-158 | MDKKYSIGLDIGTNSVGWAVITDEYKVPSKKFKVLGNTDRKSIKKNLIGALL | 118 |
| | FDSGETAEATRLKRTARRRYTRRKNRIRYLQEIFAEEMSKVDDGFFQRLEES | |
| | FLVEEDKKNERHPIFGNIVDEVAYHEKYPTIYHLRKYLADSTEKADLRLIYL | |
| | ALAHIIKFRGHFLIEGDLNSENTDVQKLFIQLVQTYNQLFEENPLNESGVDA | |
| | KAILSAKLSKSRRLENLIAQLPGEKKNGLFGNLIALSLGLTPNFKSNFDLSE | |
| | DAKLQLSKDTYDEDLDNLLAQIGDQYADLFLAAKNLSDAILLSDILTVNDES | |
| | TKAPLSASMVKRYDEHHQDLTLLKALVREQLPEKYKEIFFDESKNGYAGYID | |
| | GGASQEEFYKYIKPILEKMDGTEYLLAKINREDFLRKQRTFDNGSIPHQIHL | |
| | GELHAILRRQEDFYPFLKENQEKIEKILTFRIPYYVGPLARGNSRFAWMTRK | |
| | SDETITPWNFEEVVDKEASAQSFIERMTNFDKNLPEEKVLPKHSLLYEYFTV | |
| | YNELTKVKYVTEGMGKPEFLSGDQKEAIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEQRLKKYAHLFDKKVLKKLKRRRYTGWGRLSRKLINGIRDKQSG | |
| | KTILDFLKSDGFANRNFMQLIHDDSLTFKEIIAKAQVIGDGDSLHEVIANLA | |
| | GSPAIKKGILQSVKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEVDENDKLIREVKIITLKSKLVSDFRKDFQFYKVREINDYHHAHDAY | |
| | LNAVVGTALIKKYPKLESEFVYGDYKVYDVRKMIAKSEQEIGKATAKYFFYS | |
| | NIMNFFKSEVTLANGTIRKRPLIETNEETGEIVWDKGKDFATVRKVLSMPQV | |
| | NIVKKTEVQTGGFSKESILPKRNSDKLIARKKDWDPKKYGGFDSPTVAYSVL | |
| | VVAKIEKGKTKKLKTVKELVGITIMERSSFEKDPVAFLETKGYKDIRIDLII | |
| | KLPKYSLFELENGRKRMLASAGELQKGNELALPSKYVTFLYLASHYEKLKGK | |
| | PEDREDKLEYVEQHRHYFDEILEQIIEFSERYILADANLEKIKELYNENNDY | |
| | PIEELAENFIHLFTFTSLGAPAAFKFFDKTIDRKRYTSTTEVLNATLIHQSI | |
| | TGLYETRIDLSQLGGD | |
| CasEnd-159 | MDKKYSIGLDIGTNSVGWAVITDEYKVPSKKFKVLGNTDRHSIKKNLIGALL | 119 |
| | FDSGETAEATRLKRTARRRYTRRKNRICYLQEIFSNEMAKVDDSFFQRLEES | |
| | FLVEEDKSNDRHPIFGNIVEEVAYHEKYPTIYHLRKHLADSPEKADLRLVYL | |
| | ALAHIIKFRGHFLIEGDLDAENTDVQKLFKELVEVYDRTFEESELEEETIDA | |
| | ESILTEKLSKSRRLENLIAKFPGEKKNSFFGNLIALALGLTPNFKSNFELSE | |
| | DAKLQFSKDTYEEDLEELLGQIGDDYADLFTAAKNLYDAILLSGILTVDDNS | |
| | TKAPLSASMVKRYDEHHQDLTLLKKFVRENLPEKYKEIFFDQSKNGYAGYID | |
| | GGASQEEFYKYLKKLLEKIDGSEYFLDKIEREDFLRKQRTFDNGSIPHQIHL | |
| | QELHAIIRRQGEYYPFLKENQEKIEKILTFRIPYYVGPLARGNSRFAWLSRK | |
| | SDETITPWNFDEVVDKEKSAEKFITRMTNFDKNLPEEKVLPKHSLLYEKFTV | |
| | YNELTKVKYVTEGGKKEEFFDANMKQEIFDNVFKKYRKVTKKQLLDYLAKEF | |
| | DEFDIVDISGVEDRFNASLGTYHDLKKILGDKSFLDNPANEKILEDIIKTLT | |
| | LFEDREMIKKRLEKYSDLFDKKQLKKLERRRYTGWGRLSAKLINGIRDKETG | |
| | KTILDYLIEDGPTNRNFMQLIHDDGLSFKEEISKAQVIDDTDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-160 | MDKKYSIGLDIGTNSVGWAVITDEYKVPSKKFKVLGNTDRHSIKKNLIGALL | 120 |
| | FDSGETAEATRLKRTARRRYTRRKNRICYLQEIFSNEMAKVDDSFFQRLEES | |
| | FLVEEDKRGERHPIFGNIVDEVAYHEKYPTIYHLRKELADSTEKADLRLVYL | |
| | ALAHIIKFRGHFLIEGDLNSENTDVQKLFKQFVQVYNQTFEESHLSEETVDA | |
| | ESILTEKVSKSRRLENLIKQFPNEKKNGLFGNLIALSLGLQPNFKSNENLSE | |
| | DAKLQFSKDTYDEDLENLLGQIGDEYADLFVAAKNLYDAILLSGILTVNDES | |
| | TKAPLSASMIKRYDEHHQDLTLLKAFVRQQLPEKYKEIFFDETKNGYAGYID | |
| | GGASQEEFYKYIKPILEKVDGSEYFLDKIDREDFLRKQRTFDNGSIPHQIHL | |
| | QELHAILRRQEEFYPFLKENQEKIEKILTFRIPYYVGPLARGNSRFAWASRK | |
| | SDETITPWNFEEVVDKEASAEAFIERMTNNDKNLPEEKVLPKHSLLYEKFTV | |
| | YNELTKVKYVTEQMGKPKFFDANMKQEIFDGLFKKERKVTKKKLLDFLFKEF | |
| | DEFRIVDISGVEDAFNASLGTYHDLLKIIKDKDFLDNEENEDILEDIILTLT | |
| | LFEDREMIEERLKKYADLFDKKVLKKLKRRRYTGWGRLSKKLINGIRDKQSG | |
| | KTILDYLIDDGFANRNFMQLIHDDSLSFKEEIAKAQVIGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVADIEKGKAKKLKTVKELVGITIMERSSFEKNPVAFLESKGYQNIQEDKII | |
| | KLPKYSLFELENGRKRLLASAGELQKGNELALPQKYVTLLYHAKHYEKLKGK | |
| | DEDNEKHLEYVEQHRDEFDEILDQISEFSERYILADKNLEKIKELYEKNEDA | |
| | SIEELASSFINLLTLTALGAPAAFKFFGTTIDRKRYTSTKEILNATLIHQSI | |
| | TGLYETRIDLSKLGGD | |
| CasEnd-161 | MDKKYSIGLDIGTNSVGWAVITDEYKVPSKKFKVLGNTDRHSIKKNLIGALL | 121 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENSDVDKLFIQLVQTYNQLFEENPINEEGVDA | |
| | KAILSAKLSKSRRLENLIAQFPGEKRNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDESKNGYAGYID | |
| | GGASQEEFYKYIKPILEKMDGTEELLAKINREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQSFIERMTNFDKNLPNEKVLPKHSLLYEYFTV | |
| | YNELTKVKYVTEGMGKPEFLSGEQKQEIVDLLFKKNRKVTVKQLKEFLFKKI | |
| | ECFDSVEISGVEDRFNASLGTYHDLLKIIKDKDFLDNEENEDILEDIVLTLT | |
| | LFEDKEMIEERLKKYANLFDKKVLKQLKRRRYTGWGRLSKKLINGIRDKQSG | |
| | KTILDFLKSDGFANRNFMQLIHDDSLTFKEEIAKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRKSDKLIARKKDWDPKKYGGFDSPTVAYSVL | |
| | VVAKVEKGKSKKLKTVKELVGITIMERSSFEKDPVDFLEAKGYKEVQKDLII | |
| | KLPKYSLFELENGRKRMLASAGELQKGNELALPSKYVILLYLASHYEKLKGS | |
| | PEDNEQHREYVEQHRHYFDEILDQISEFSERYILADKNLEKILELYSEFEDY | |
| | SIEEQAESFINLFTLTALGAPAAFKFFDTTIDRKRYTSTKEILNATLIHQSI | |
| | TGLYETRIDLSQLGGD | |
| CasEnd-162 | MDKKYSIGLDIGTNSVGWAVITDEYKVPSKKFKVLGNTDRQSIKKNLIGALL | 122 |
| | FDSGETAEATRLKRTARRRYTRRKNRIRYLQEIFAEEMSKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSENSDVDKLFIQLVQTYNQLFEENPINESGVDA | |
| | KAILSAKLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SDETITPWNFEEVVDKGASAQSFIERMTNFDKNLPEEKVLPKHSLLYEYFTV | |
| | YNELTKVKYVTEGMGKPEFLSGEQKKEIVDLLFKKNRKVTVKQLKEFYFKEI | |
| | ECFDSVEISGVEDRFNASLGTYHDLLKIIKDKDFLDNEENEDILEDIVLTLT | |
| | LFEDREMIEERLKKYANLFDKKVMKQLKRRRYTGWGRLSRKLINGIRDKQSG | |
| | KTILDFLKSDGFANRNFMQLIHDDSLTFKEEIEKAQVKGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGQELDINR | |
| | LSDYDVDHIVPQSFLKDDSIDNKVLTRSKKARGKSDNVPSEEVVKKMKSYWR | |
| | QLLKAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPVIETNEETGEIVWDKERDFATVRKVLSMPQV | |
| | NIVKKTEVQTGGFSKESILPKRESDKLIARKKDWDPKKYGGFDSPTVAYSVL | |
| | VVAKIEKGKTKKLKTVKELLGITIMERSSFEKDPVAFLEAKGYKEVKKDLII | |
| | KLPKYSLFELENGRKRMLASAGELQKGNELALPSKYVNFLYLASHYEKLKGS | |
| | PEDNEQKQLFVEQHKDYLDEIIDQISEFSERVILADKNLEKVLSAYNENRDK | |
| | SIEEQAENIIHLFTLTNLGAPAAFKYFDTTIDRKRYTSTKEVLDATLIHQSI | |
| | TGLYETRIDLSQLGGD | |
| CasEnd-163 | MDKKYSIGLDIGTNSVGWAVITDEYKVPSKKFKVLGNTDRHSIKKNLIGALL | 123 |
| | FDSGETAEATRLKRTARRRYTRRKNRILYLQEIFSNEMAKVDDSFFHRLEES | |
| | FLVEEDKRHERHPIFGNIVDEVAYHEKYPTIYHLRKKLADSTEKADLRLIYL | |
| | ALAHMIKFRGHFLIEGDLNSDNSDVQKLFIQLVQTYNQLFEENPINEEGVDA | |
| | KAILTARLSKSRRLENLIAQLPGEKKNGLFGNLIALSLGLTPNFKSNEDLAE | |
| | DAKLQLSKDTYDEDLENLLAQIGDQYADLFLAAKNLSDAILLSGILTVNTEI | |
| | TKAPLSASMVKRYDEHHQDLTLLKQLVRKQLPEKYKEIFFDQSKNGYAGYID | |
| | GGASQEEFYKYLKPILEKMDGTEEFLAKINREDFLRKQRTFDNGSIPHQIHL | |
| | GELHAILRRQEEYYPFLKENREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMRKPEFLSAEQKQEIVDLLFKKNRKVTVKQLKEDYFKKF | |
| | ECFDSVEISGVEDRFNASLGTYHDLLKIIKDKDFLDNEENEKILEDIVLTLT | |
| | LFEDREMIEERLKKYADLFDDKQLKQLKRRRYTGWGRLSRKLINGIRDKQSG | |
| | KTILDFLKADGFANRNFMQLIHDDSLTFKEEIQKAQVSGQGDSLHETIANLA | |
| | GSPAIKKGILQTLKIVDELVKVMGRHEPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSSFEKNPIAFLEAKGYKEVKKDLII | |
| | KLPKYSLFELENGRKRMLASAGELQKGNELALPNKYVNLLYLASHYEKLKGS | |
| | PEDNEQKQLFVEQHKHYFDEIIEQISEFSKRVILADANLEKIKSLYEKNRDK | |
| | SIEELAENFIHLFTFTALGAPAAFKFFDTTIDRKRYTSTKEILNATLIHQSI | |
| | TGLYETRIDLSQLGGD | |
| CasEnd-164 | MDKKYSIGLDIGTNSVGWAVITDDYKVPSKKFKVLGNTDRQSIKKNLIGALL | 124 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSENSDVDKLFIQLVQTYNQLFEENPINEEGVDA | |
| | KAILSAKLSKSRRLENLIAQFPGEKKNGLFGNLIALSLGLTPNFKSNEDLSE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDESKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SDETITPWNFEEVVDKGASAQSFIERMTNFDENLPEEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDDKVMKQLKRRRYTGWGRLSRKLINGIRDKQSG | |
| | KTILDFLKSDGFANRNFMQLIHDDSLTFKEEIEKAQVKGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNDKLYLYYLQNGRDMYTGQELDINR | |
| | LSQYDVDHIVPQSFLKDDSIDNKVLTRSAKARGKSDNVPSEEVVKKMKNYWR | |
| | QLLKAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKKYDVRKMIAKSEQEIGKATAKYFFYS | |
| | NIMNFFKTEIKLANGEIRKRPLIETNEETGEIVWDKERDFATVRKVLSMPQV | |
| | NIVKKTEVQTGGFSKESILPKRESDKLIARKKGWDPKKYGGFDSPTVAYSVL | |
| | VVAKVEKGKTKKLKTVKELLGITIMERSSFEKDPVAFLEAKGYKDVKKDLII | |
| | KLPKYSLFELENGRKRMLASAGELQKGNELALPSKYVNFLYLASHYESLKGS | |
| | PEDNEKKQEYVEQHKHYLDEIIDQISEFSERVILADANLEKVLSAYNNERDK | |
| | SIEEQAENIIHLFTLTNLGAPAAFKYFDTTIDRKRYTSTKEVLDATLIHQSI | |
| | TGLYETRIDLSQLGGD | |
| CasEnd-165 | MDKKYSIGLDIGTNSVGWAVITDEYKVPSKKFKVLGNTDRHSIKKNLIGALL | 125 |
| | FDSGETAEATRLKRTARRRYTRRKNRICYLQEIFSNEMAKVDESFFHRLEES | |
| | FLVEEDKSFERHPIFGNIVEEVAYHEKYPTIYHLRKKLVDSPEKADLRLIYL | |
| | ALAHMIKFRGHFLIEGDLNSDNSDVQKLFKQLVQTYNQLFEESPIEAEGVDA | |
| | KSILSEKLSKSRRLENLIAQLPGEKKNGLFGNLIALSLGLTPNFKSNEDLSE | |
| | DAKLQLSKDTYDDDLEELLGQIGDEYADLFLAAKNLSDAILLSGILRVDTES | |
| | TKAPLSASMIKRYDEHHQDLTLLKQLVREQLPEKYKEIFFDQSKNGYAGYID | |
| | GGASQEEFYKYIKKILEKMDGTEELLDKLEREDLLRKQRTFDNGSIPHQIHL | |
| | GELHAILRRQEEYYPFLKENREKIEKILTFRIPYYVGPLARGNSRFAWMSRK | |
| | SDETITPWNFDEVVDKGASAEKFIERMTNFDKNLPEEKVLPKHSLLYEYFTV | |
| | YNELTKVKYVTEGMRKPEFLDGNQKKEIVDDLFKKNRKVTVKQLKEYYFKKE | |
| | ECFDSVEISGVEDRFNASLGTYHDLLKIIKDKSFLDNDENEKILEDIVLTLT | |
| | LFEDREMIKKRLEKYADLFDKKVMKQLKRRRYTGWGRLSKKLINGIRDKQSG | |
| | KTILDFLIDDGFANRNFMQLIHDDSLTFKDEIAKAQVIGKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-166 | MDKKYSIGLDIGTNSVGWAVITDEYKVPSKKFKVLGNTDRHSIKKNLIGALL | 126 |
| | FDSGETAEATRLKRTARRRYTRRKNRICYLQEIFSNEMAKVDDSFFHRLEES | |
| | FLVEEDKRNERHPIFGNIVEEVAYHEKYPTIYHLRKKLADSDEKADLRLVYL | |
| | ALAHIIKFRGHFLIEGDLDAENTDVQKLFRELVEVYNQTFEESPLEEITVDA | |
| | EAILTEKLSKSRRLENLIAQFPGEKKNGLFGNLLALALGLTPNFKSNEDLEE | |
| | DAKLQFSKDTYDEDLEELLGQIGDEYADLFLAAKKLYDAILLSGILTVDDES | |
| | TKAPLSASMVKRYDEHHQDLTLLKQFIRKKLPEKYKEIFFDQSKNGYAGYID | |
| | GGASQEEFYKYLKKILEKIDGSEEFLDKIEREDFLRKQRTFDNGSIPHQIHL | |
| | QELHAIIRRQEEYYPFLKENREKIEKILTFRIPYYVGPLARGNSRFAWLSRK | |
| | SDETITPWNFDEVVDKEKSAEAFIERMTNFDKNLPEEKVLPKHSLLYEKFTV | |
| | YNELTKVKYVTEGGKKPEFFDAEQKQEIFDNLFKKERKVTKKQLKDYLFKEF | |
| | DEFRIVEISGVEDRFNASLGTYHDLLKIIKDKDFLDNPENEEILEDIILTLT | |
| | LFEDREMIKKRLEKYADLFDKKQLKKLSRRRYTGWGRLSAKLINGIRDKQTG | |
| | KTILDYLIDDGFANRNFMQLIHDDSLTFKEEIEKAQVIGDGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADVEKGKAKKLKTVKELVGITIMERSAFEKNPVAFLEDKGYQNIKKELII | |
| | KLPKYSLFELENGRKRLLASAGELQKGNELALPQHYVILLYHAKHYEKLKGS | |
| | PEDNEYKQLYVEEHKDEFDEILDQIIEFSKRYILADANLEKIKSLYEKNKDA | |
| | SIEELAENFIHLLTFTALGAPAAFKFFGKTIDRKRYTSTKEILNATLIHQSI | |
| | TGLYETRIDLSKLGGD | |
| CasEnd-167 | MDKKYSIGLDIGTNSVGWAVITDEYKVPSKKFKVLGNTDRHSIKKNLIGALL | 127 |
| | FDSGETAEATRLKRTARRRYTRRKNRICYLQEIFSNEMAKVDDSFFHRLEES | |
| | FLVEEDKRGERHPIFGNIVEEVAYHEKYPTIYHLRKHLADSTEKADLRLVYL | |
| | ALAHIIKFRGHFLIEGDLNAENSDVQKLFKQLVEVYDQTFEESPLSEITVDA | |
| | KAILTEKLSKSRRLENLIKQFPNEKKNGLFGNLIALSLGLQPNFKTNENLSE | |
| | DAKLQFSKDTYDEDLENLLGQIGDEYADLFLAAKNLYDAILLSGILTVNDES | |
| | TKAPLSASMIKRYDEHHQDLTLLKQFIRKQLPEKYKEIFFDESKNGYAGYID | |
| | GGASQEEFYKYIKKLLEKMDGSEYFLDKIEREDFLRKQRTFDNGSIPHQIHL | |
| | QELHAILRRQEEYYPFLKENQEKIEKILTFRIPYYVGPLARGNSRFAWMTRK | |
| | SDETITPWNFDEVVDKEKSAEAFIERMTNNDKNLPEEKVLPKHSLLYEKFTV | |
| | YNELTKVKYVTEGMGKAQFFDANQKQEIFDGLFKKYRKVTKKKLLDFLDKEF | |
| | DEFRIVDISGVEDAFNASLGTYHDLLKIIKDKDELDNPENEDILEDIILTLT | |
| | LFEDREMIEKRLSKYADLFDKKVLKKLKRRRYTGWGRLSKKLINGIRDKQSG | |
| | KTILDYLIDDGFANRNFMQLIHDDSLSFKEEIKKAQVIGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKAKKLKTVKELIGITIMERSSFEKNPVAFLEDKGYKNIQEETII | |
| | KLPKYSLFELENGRKRLLASAGELQKGNELALPNKYVTLLYHAKHYEKLKGK | |
| | PEDEEKHLEYVEKHRDEFNEILDQISEFSERYILADKNLSKINELYKKNNDK | |
| | SIEELASSFINLLTFTALGAPAAFKFLGTTIDRKRYTSTKEILNATLIHQSI | |
| | TGLYETRIDLSKLGGD | |
| CasEnd-168 | MDKPYSIGLDIGTNSVGWAVVTDEYKVPSKKFKVLGNTDRHSIKKNLLGALL | 128 |
| | FDSGETAEATRLKRTARRRYTRRKNRILYLQEIFAEEMAKVDDSFFHRLEES | |
| | FLVEEDKRGERHPIFGNIVEEVAYHEKYPTIYHLRKKLADSDEKADLRLVYL | |
| | ALAHIIKFRGHFLIEGDLNAENSDVQKLFDQLVQTYNRLFEESPLEEEEVDA | |
| | EAILTEKLSKSRRLENLIAQFPGEKKNGLFGNLIALSLGLTPNFKSNFDLAE | |
| | DAKLQFSKDTYDEDLEELLAQIGDEYADLFLAAKNLYDAILLSGILTVSDES | |
| | TKAPLSASMVKRYDEHHQDLTLLKKFIRKQLPEKYKEIFFDQSKNGYAGYID | |
| | GGASQEEFYKYLKKILEKIDGSEEFLDKIEREDFLRKQRTFDNGSIPHQIHL | |
| | QELHAILRRQEKYYPFLKENQEKIEKILTFRIPYYVGPLARGNSRFAWMSRK | |
| | SDETITPWNFEEVVDKEASAEKFIERMTNFDKNLPNEKVLPKHSLLYEKFTV | |
| | YNELTKVKYVTEGMRKPKFFSAEQKQEIVDLLFKKNRKVTKKQLKEYLKKEF | |
| | ECFDIVEISGVEDRFNASLGTYHDLLKIIKDKDFLDNEENEEILEDIVLTLT | |
| | LFEDREMIKKRLEKYADLFDKKQLKKLKRRRYTGWGRLSAKLINGIRDKQSG | |
| | KTILDYLIDDGFANRNFMQLIHDDSLTFKEEIAKAQVIGQGDSLHEVIANLA | |
| | GSPAIKKGILQSLKIVDELVKVMGRHEPENIVIEMARENQTTQKGQKNSRER | |
| | MKRLEEAIKELGSKILKEHPVENTKLQNDKLYLYYLQNGKDMYTGEELDIDR | |
| | LSDYDVDHIVPQSFLKDDSIDNKVLVSSKKARGKSDDVPSEEVVKKMKGYWK | |
| | KLLDAKLITQRKFDNLTKAERGGLTELDKAGFIKRQLVETRQITKHVAQILD | |
| | SRFNTEYDENDKLIRDVKIITLKSKLVSQFRKDFELYKVREINNYHHAHDAY | |
| | LNAVVGTALIKKYPKLESEFVYGDYKVYDVRKMIAKSEQEIGKATAKYFFYS | |
| | NIMNFFKTEVKLANGEIRKRPLIEVNEETGEIVWDKEKDFATVRKVLSMPQV | |
| | NIVKKTEVQTGGFSKESILPKRDSDKLIARKKDWDTKKYGGFDSPTVAYSVL | |
| | VIAKVEKGKAKKLKTVKELVGITIMERSAFEKNPIAFLEDKGYQNIKKDLII | |
| | KLPKYSLFELENGRKRLLASAGELQKGNELALPNKYVELLYHAKHYEKLKGK | |
| | PEDNEEKQLYVEQHKSYFDEILEQISEFSKRYILADANLEKIKKLYEKNRDA | |
| | SIEELAESFINLLTFTALGAPAAFKFFDTTIDRKRYTSTKEILNATLIHQSI | |
| | TGLYETRIDLSKLGGD | |
| CasEnd-169 | MDKKYSIGLDIGTNSVGWAVVTDDYKVPSKKFKVLGNTDRKSIKKNLLGALL | 129 |
| | FDSGETAEATRLKRTARRRYTRRKNRICYLQEIFSNEMAKVDESFFHRLEES | |
| | FLVEEDKRGERHPIFGNIVEEVAYHEKFPTIYHLRKELADSPEKADLRLVYL | |
| | ALAHIIKFRGHFLIEGDLNSENTDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNDES | |
| | TKAPLSASMVKRYEEHHKDLTLLKQFIRKQLPEKYKEIFFDKSKNGYAGYID | |
| | GGASQEEFYKYIKKILEKIDGSEEFLDKIEREDFLRKQRTFDNGSIPHQIHL | |
| | QELHAILRRQEKYYPFLKENQEKIEKILTFRIPYYVGPLARGNSRFAWLSRK | |
| | SEETITPWNFDEIVDKEASAEAFIERMTNFDKNLPNEKVLPKHSLLYEKFTV | |
| | YNELTKVKYVTEGGKKPKFFDANQKQEIVDLLFKKNRKVTKKQLKDFLNKEF | |
| | DEFRIVEISGVEDRFNASLGTYHDLLKIIGDKDFLDNSENEEILEDIILTLT | |
| | LFEDREMIKKRLEKYADLFDKEQLKKLERRRYTGWGRLSAKLINGIRDKQSG | |
| | KTILDYLIDDGFANRNFMQLIHDDSLSFKEEIAKAQVIGDTDSLHEIIANLA | |
| | GSPAIKKGILQSIKIVDELVKVMGRYAPENIVVEMARENQTTAKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLTDDDKAGFIKRQLVETRQITKHVAQILD | |
| | SRFNTEYDENGKLIRKVKIVTLKSKLVSDFRKDFELYKVREINDYHHAHDAY | |
| | LNAVVGKALIKKYPKLESEFVYGDYPKYNSRKMIAKSDRERGKATAKMFFYS | |
| | NIMNFFKSDVKLADGEIRERPLIEVNEETGEIVWDKVKHIATVKKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKDPVAFLEDKGYQNIQEDNII | |
| | KLPKYSLFELENGRKRLLASAGELQKGNELALPNHLVTLLYHAKHIEKLKGK | |
| | PEDEEEKLSYVEQHREEFDELLDQIIEFSKRYILADANLEKIKKLYEKNEEA | |
| | DIEELASSFINLLTFTALGAPAAFKFFDKTIDRKRYTSTKEILNATLIHQSI | |
| | TGLYETRIDLSKLGGD | |
| CasEnd-170 | MDKKYSIGLDIGTNSVGWAVITDEYKVPSKKFKVLGNTDRQSIKKNLIGALL | 130 |
| | FDSGETAEATRLKRTARRRYTRRKNRIRYLQEIFSEEMSKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNPENSDVQKLFIQLVQTYNQLFEENPINESGVDA | |
| | KAILSAKLSKSRRLENLIAQLPGEKKNGLFGNLIALSLGLTPNFKSNEDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKYIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPNEKVLPKHSLLYEYFTV | |
| | YNELTKVKYVTEGMRKPEFLSGEQKEEIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLEKYAHLFDKKVLKQLKRRRYTGWGRLSRKLINGIRDKQSG | |
| | KTILDFLKSDGFANRNFMQLIHDDSLTFKEEIQKAQVIGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKNPVDFLEAKGYKNVQKDLII | |
| | KLPKYSLFELENGRKRLLASAGELQKGNELALPQKYVTLLYLASHYEKLKGS | |
| | SEDNEKKLEFVEQHRHYFDEIIEQISEFSERYILADKNLEKILSLYDEFEDY | |
| | SIEELAENFIHLFTFTSLGAPAAFKFFDTTIDRKRYTSTTEILDATLIHQSI | |
| | TGLYETRIDLSQLGGD | |
| CasEnd-171 | MDKKYSIGLDIGTNSVGWAVVTDEYKVPSKKFKVLGNTDRHSIKKNLIGALL | 131 |
| | FDSGETAEATRLKRTARRRYTRRKNRIRYLQEIFAEEMAKVDDSFFHRLEES | |
| | FLVEEDKRNERHPIFGNIVDEVAYHEKYPTIYHLRKKLADSDEKADLRLVYL | |
| | ALAHIIKFRGHFLIEGDLNAENTDVQKLFEQLVETYNQLFEESPLDEEKVDA | |
| | KAILTEKLSKSRRLENLIAQFPGEKKNGLFGNLLALSLGLTPNFKSNEDLSE | |
| | DAKLQFSKDTYDEDLEELLGQIGDEYADLFLAAKNLYDAILLSGILTVNDES | |
| | TKAPLSASMVKRYDEHHQDLTLLKKFIRQQLPEKYKEIFFDQSKNGYAGYID | |
| | GGASQEEFYKYLKPILEKIDGSEEFLDKIEREDFLRKQRTEDNGSIPHQIHL | |
| | QELHAILRRQEKYYPFLKENQEKIEKILTFRIPYYVGPLARGNSRFAWLSRK | |
| | SDETITPWNFDEVVDKEASAQAFIERMTNFDKNLPNEKVLPKHSLLYEKFTV | |
| | YNELTKVKYVTEGMRKPEFFSGEQKQEIFDLLFKKNRKVTKKQLKEYLFKEF | |
| | ECFDSVEISGVEDRFNASLGTYHDLLKIIKDKDFLDNEANEEILEDIILTLT | |
| | LFEDREMIKKRLKKYADLFDKKVLKQLKRRRYTGWGRLSAKLINGIRDKQSG | |
| | KTILDYLIDDGFANRNFMQLIHDDSLSFKEEIEKAQVIGDGESLHEVIANLA | |
| | GSPAIKKGILQSLKIVDELVKVMGRHAPENIVIEMARENQTTQKGQKNSRER | |
| | MKRLEEAIKELGSNILKEHPVENTQLQNDKLYLYYLQNGKDMYTGEELDIDR | |
| | LSDYDVDHIVPQSFLKDDSIDNRVLVSSAKARGKSDNVPSEEVVKKMKPYWK | |
| | QLLDAKLITQRKFDNLTKAERGGLTELDKAGFIKRQLVETRQITKHVAQILD | |
| | SRFNTKYDENDKLIRDVKIITLKSKLVSQFRKDFELYKVREINNYHHAHDAY | |
| | LNAVVGTALIKKYPKLESEFVYGDYKVYDVRKMIAKSEQEIGKATAKYFFYS | |
| | NIMNFFKTEVKLANGEIRKRPLIEVNEETGEIAWDKEKDFATVKKVLSMPQV | |
| | NIVKKTEVQTGGFSKESILPKRDSDKLIARKKDWDTKKYGGFDSPTVAYSVL | |
| | VIAKVEKGKAKKLKTVKELVGITIMERSAFEKNPVAFLEAKGYKNIKKDLII | |
| | KLPKYSLFELENGRKRLLASAGELQKGNELALPNKYVTLLYHASHYEKLKGS | |
| | PEDNEEKQLYVEQHKDYFDEILEQISEFSKRYILADANLEKIKKLYEKNRDL | |
| | SIEELAESFINLLTFTALGAPAAFKFFDTTIDRKRYTSTKEILNATLIHQSI | |
| | TGLYETRIDLSKLGGD | |
| CasEnd-172 | MKKKYSIGLDIGTNSVGWAVITDEYKVPSKKFKVLGNTDRQSIKKNLIGALL | 132 |
| | FDSGETAEATRLKRTARRRYTRRKNRIRYLQEIFAEEMSKVDDSFFHRLEES | |
| | FLVEEDKKHERHPIFGNIVDEVAYHEDYPTIYHLRKKLADSTEKADLRLVYL | |
| | ALAHMIKFRGHFLIEGDLNSENSDVDKLFIQLVQTYNQLFEENPINESHVDA | |
| | KAILSAKLSKSRRLENLIAQFPNEKKNGLFGNLIALSLGLTPNFKSNFQLSE | |
| | DAKLQLSKDTYDEDLENLLAQIGDQYADLFLAAKNLSDAILLSDILTVNDES | |
| | TKAPLSASMIKRYDEHHQDLTLLKALVREQLPEKYKEIFFDQSKNGYAGYID | |
| | GGASQEEFYKYIKPILEKMDGSEELLTKINREDFLRKQRTFDNGSIPHQIHL | |
| | QELHAILRRQEKYYPFLKENQEKIEKILTFRIPYYVGPLARGNSRFAWMSRK | |
| | SEETITPWNFEEVVDKEASAQAFIERMTNFDKNLPEEKVLPKHSLLYEKFTV | |
| | YNELTKVKYVTEGMGKPEFLSGEQKQEIVDLLFKKNRKVTVKQLKEDLFKKI | |
| | DCFDSVEISGVEDRFNASLGTYHDLLKIIKDKEFLDNEENEKILEDIVLTLT | |
| | LFEDREMIEERLKKYADLFDKKVLKQLKRRRYTGWGRLSKKLINGIRDKQSG | |
| | KTILDFLKDDGFANRNFMQLIHDDSLTFKEEIQKAQVEGQSDSLHEQIADLA | |
| | GSPAIKKGILQSVKIVDELVKVMGRHKPENIVIEMARENQTTQKGQKNSRER | |
| | MKRLEEGIKELGSQILKEHPVENTQLQSEKLYLYYLQNGKDMYTGEELDIDR | |
| | LSDYDVDHIIPQSFIKDDSIDNKVLTSSEKARGKSDDVPSEEVVKKMKSYWR | |
| | KLLKAKLISQRKFDNLTKAERGGLSELDKAGFIKRQLVETRQITKHVAQILD | |
| | SRFNTEYDENDKLIRDVKIITLKSKLVSDFRKDFQFYKVREINDYHHAHDAY | |
| | LNAVVGTALIKKYPKLESEFVYGDYKVYDVRKMIAKSEQEIGKATAKMFFYS | |
| | NIMNFFKTEITLANGEIRKRPLIETNEETGEIVWDKEKDFATVRKVLSMPQV | |
| | NIVKKTEVQTGGFSKESILPKRESDKLIARKKDWDPKKYGGFDSPTVAYSVL | |
| | VVAKVEKGKSKKLKTVKELVGITIMERSSFEKNPVAFLEKKGYKNVQKDLII | |
| | KLPKYSLFELENGRKRLLASAGELQKGNELALPQKYVTLLYHAKHYEKLKGS | |
| | PEDNEKHREYVEQHKDYFDEILDQIEEFSKRYILADKNLDKILSLYSKNEDA | |
| | PIEELAESFINLFTFTALGAPAAFKFFGTTIDRKRYTSTKEILDATLIHQSI | |
| | TGLYETRIDLSKLGGD | |
| CasEnd-173 | MDKKYSIGLDIGTNSVGWAVITDEYKVPSKKFKVLGNTDRHSIKKNLIGALL | 133 |
| | FDSGETAEATRLKRTARRRYTRRKNRIRYLQEIFAEEMAKVDDSFFHRLEES | |
| | FLVEEDKRNERHPIFGNIVDEVAYHEKYPTIYHLRKKLADSDEKADLRLIYL | |
| | ALAHMIKFRGHFLIEGDLNADNSDVQKLFIQLVQTYNQLFEENPLNEETVDA | |
| | KAILTAKLSKSRRLENLIAQFPGEKKNGLFGNLIALSLGLTPNFKSNFDLAE | |
| | DAKLQLSKDTYDEDLENLLAQIGDQYADLFLAAKNLSDAILLSGILTVNDES | |
| | TKAPLSASMIKRYDEHHQDLTLLKKLVRQQLPEKYKEIFFDQSKNGYAGYID | |
| | GGASQEEFYKYLKPILEKVDGSEEFLDKIEREDFLRKQRTFDNGSIPHQIHL | |
| | GELHAILRRQEKYYPFLKENREKIEKILTFRIPYYVGPLARGNSRFAWMTRK | |
| | SDETITPWNFEEVVDKEASAQDFIERMTNFDKNLPNEKVLPKHSLLYEKFTV | |
| | YNELTKVKYVTEGMRKPEFLSAEQKQEIVDLLFKKNRKVTVKQLKEYLFKEF | |
| | ECFDSVEISGVEDRFNASLGTYHDLLKIIKDKDELDNEENEDILEDIVLTLT | |
| | LFEDREMIEERLKKYADLFDDKVLKQLKRRRYTGWGRLSKKLINGIRDKQSG | |
| | KTILDFLIDDGFANRNFMQLIHDDSLTFKEEIEKAQVSGQGESLHELIANLA | |
| | GSPAIKKGILQSIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKGKDFATVRKVLSMPQV | |
| | NIVKKTEVQTGGFSKESILPKRDSDKLIARKKDWDTKKYGGFDSPTVAYSVL | |
| | VVAKVEKGKAKKLKTVKELVGITIMERSSFEKNPVAFLEAKGYKNVKKDLII | |
| | KLPKYSLFELENGRKRLLASAGELQKGNELALPSKYVTLLYLASHYEKLKGS | |
| | PEDNEQKQLFVEQHKDYFDEIIEQISEFSKRYILADANLEKIKSLYEKNRDK | |
| | SIEELAENFIHLLTFTNLGAPAAFKFFDTTIDRKRYTSTKEILNATLIHQSI | |
| | TGLYETRIDLSQLGGD | |
| CasEnd-174 | MKKPYSIGLDIGTNSVGWAVITDDYKVPSKKMKVLGNTDRQSIKKNLLGALL | 134 |
| | FDSGETAEATRLKRTARRRYTRRKNRILYLQEIFAEEMNKVDDSFFHRLEDS | |
| | FLVEEDKRGERHPIFGNIVEEVKYHEKFPTIYHLRKKLADSTEKADLRLVYL | |
| | ALAHIIKFRGHFLIEGDLNAENTDVQKLFKEFVEVYDQTFEESHLVEETIDA | |
| | EMILTEKISKSRRLENLIEQFPGEKKNGLFGNLIALSLGLQPNFKSNEDLSE | |
| | DAKLQFSKDTYDEDLENLLGQIGDEYADVFLAAKNLYDAILLSGILTVNDSS | |
| | TKAPLSASMIKRYDEHHEDLTLLKDFVRENLPEKYKEIFFDESKNGYAGYID | |
| | GGTSQEEFYKYIKPILNKVDGSEYFLDKIDREDFLRKQRTFDNGSIPHQIHL | |
| | YELHAILRRQEKFYPFLKENQEKIEKILTFRIPYYVGPLARGNSRFAWMSRK | |
| | SDETITPWNFDEVVDKEASAEAFIERMTNNDLYLPDEKVLPKHSLLYEKFTV | |
| | YNELTKVKYVTEGMGKAFFFDANQKQEIFDLLFKKNRKVTKKKLLEFLFKEF | |
| | DEFRIVDISGVEKAFNASLGTYHDLLKIIKDKDELDNPENEDILEDIILTLT | |
| | LFEDREMIKERLSKYADLFDKKVLKKLKRRHYTGWGRLSAKLINGIRDKQSG | |
| | KTILDYLIDDGFANRNFMQLIHDDSLSFKEEIQKAQVIGQTDSLHEVVANLA | |
| | GSPAIKKGILQSVKIVDELVKVMGRYNPENIVIEMARENQTTAKGQRNSRER | |
| | LKKLEEAIKELGSQILKEHPVENQQLQNDRLYLYYLQNGKDMYTGEELDIDN | |
| | LSQYDVDHIIPQSFIKDDSIDNRVLVSSAKARGKSDNVPSIEVVKKMKSFWR | |
| | KLLNAKLISQRKFDNLTKAERGGLTEDDKAGFIKRQLVETRQITKHVAQILD | |
| | SRFNTETDENHKLIRKVKIITLKSKLVSDFRKDFGFYKVREINDYHHAHDAY | |
| | LNAVVGKALIKKYPKLEPEFVYGDYPKYNSYKLIAKSEKEEGKATAKKFFYS | |
| | NIMNFFKTEIKLADGTIRERPVIEVNEETGEIVWDKTKHFATVRKVLSMPQV | |
| | NIVKKTEVQTGGFSKESILPKRDSDKLIARKKKWDTKKYGGFDSPTVAYSVL | |
| | VVAKIEKGKNKKLKTVKELVGITIMERSRFEKDPVAFLEDKGYKNVQEDTII | |
| | KLPKYSLFELENGRKRLLASAGELQKGNELALPAHLVTLLYHAKRIEKLDES | |
| | KEDKPKHREYVEQHRHEFDEILDQISEFSNRYILADKNLEKIESLYANNVSA | |
| | SIEELASSFINLLTFTALGAPADFKFFGGTIDRKRYTSTKECLNATLIHQSI | |
| | TGLYETRIDLSKLGGD | |
| CasEnd-175 | MKKKYSIGLDIGTNSVGWAVVTDEYKVPSKKFKVLGNTDRQSIKKNLLGALL | 135 |
| | FDSGETAEATRLKRTARRRYTRRKNRILYLQEIFAEEMNKVDDSFFHRLEES | |
| | FLVEEDKRGERHPIFGNIVEEVAYHEKYPTIYHLRKHLADSDEKADLRLVYL | |
| | ALAHIIKFRGHFLIEGDLNAENTDVQELFKQFLQVYDLTFEEDHLSEETIDA | |
| | EEILTEKVSKSRKLENLLAQFPGEKKNGLFGNLLKLSLGLQPNFKKNENLSE | |
| | DAKLQFSKDTYDEDLENLLGQIGDEYADLFVAAKNLYDAILLSGILTVDDLS | |
| | TKAPLSASMIKRYDEHHQDLTKLKEFVRENLPEKYKEIFFDKSKNGYAGYID | |
| | GGASQEDFYKYLKKLLEKVAGAEYFLEKIEREDFLRKQRTFDNGSIPHQIHL | |
| | QELHAILRRQGEYYPFLKENQEKIEKILTFRIPYYVGPLARGNSRFAWASRK | |
| | SDETITPWNFDEVVDKEKSAEAFIERMTNNDKNLPEEKVLPKHSLLYEKFTV | |
| | YNELTKVKYVTEQMGKSQFFDANMKQEIFDGLFKKERKVTKKKLLDFLDKEF | |
| | DEFRIVDISGVEKAFNASLGTYHDLLKILKDKEFLDNPENEKILEDIVLTLT | |
| | LFEDREMIKKRLRKYADLFTKKQLKKLERRRYTGWGRLSAKLINGIRDKQSG | |
| | KTILDYLIDDGLANRNFMQLIHDDNLSFKDEIAKAQVIGQSDSLHEVIADLA | |
| | GSPAIKKGILQSIKIVDELVKVMGRYEPENIVVEMARENQTTQKGQRNSRER | |
| | LKRLEDALKELGSKILKEHPVENTQLQNDKLYLYYLQNGKDMYTGEELDIDN | |
| | LSDYDVDHIIPQSFIKDDSIDNRVLVSSAEARGKSDDVPSIEVVKKMKPFWE | |
| | KLLKAKLISQRKFDNLTKAERGGLTELDKAGFIKRQLVETRQITKHVAQILD | |
| | ERFNTEFDENNKLIRKVKIVTLKSKLVSNFRKEFGFYKVREINDYHHAHDAY | |
| | LNAVVGKALIKKYPKLESEFVYGDYPVYNSYKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-176 | MDKPYSIGLDIGTNSVGWAVVTDEYKVPSKKFKVLGNTDRHSIKKNLIGALL | 136 |
| | FDSGETAEATRLKRTARRRYTRRKNRIRYLQEIFSEEMVKVDDSFFHRLEES | |
| | FLVEEDKRHERHPIFGNIVEEVAYHEDYPTIYHLRKTLADSTEKADLRLVYL | |
| | ALAHIIKFRGHFLIEGDLDSENSDVQVLFKVLVQTYNILFEENHLSEETVDA | |
| | KAILTDKVSKSRRLENLIKQFPGEKKNGLFGNIIALSLGLTPNFKSNFDLAE | |
| | DAKLQFSKDTYDEDLENLLAQIGDQYADLFLAAKNLYDAILLSGILTVNTEI | |
| | TKAPLSASMIKRYDEHHQDLTLLKKFIRVQLPEKYKEIFFDQSKNGYAGYID | |
| | GGASQEDFYKYIKNILSKLDGTEYFLDKIEREDFLRKQRTFDNGSIPHQIHL | |
| | QELRAILRRQEKFYPFLKENQEKIEKILTFRIPYYVGPLARGNSRFAWATRK | |
| | SNETITPWNFEEVVDKEASAQAFIERMTNFDKNLPNEKVLPKHSLLYEKFTV | |
| | YNELTKVKYVTEGMRKPAFLSSEQKKAIVDLLFKKNRKVTVKQLKEFLFKKI | |
| | DCFDSVEISGVEDRFNASLGTYHDLLKIIKDKEFLDNEENEDILEDIVLTLT | |
| | LFEDREMIEERLKKYADLFDKKVIKQLKRRRYTGWGRLSAKLINGIRDKQSG | |
| | KTILDYLIDDGFANRNFMQLIHDDSLSFKEEIKKAQVIGQTDSLHQVIANLA | |
| | GSPAIKKGILQTIKIVDELVKVMGRYAPENIVIEMARENQTTQKGQRNSRER | |
| | LKRLEEAIKELGSSILKENPVENTQLQNDKLYLYYLQNGKDMYTGEELDIDR | |
| | LSDYDVDHIIPQSFIKDDSIDNRVLTSSEKARGKSDDVPSEEVVKKMKSYWS | |
| | KLLRAKLISQRKFDNLTKAERGGLSELDKAGFIKRQLVETRQITKHVAQILD | |
| | SRFNTKYDENDKLIRTVKIITLKSKLVSDFRKDFEFYKVREINDYHHAHDAY | |
| | LNAVVGTALIKKYPKLESEFVYGDYKVYNSYKMIAKSEQEIGKATAKYFFYS | |
| | NIMNFFKSDITLANGEIRKRPLIETNEETGEIAWNKTKDFATVKKVLSMPQV | |
| | NIVKKTEVQTGGFSKESILPKRESDKLIARKKDWDPKKYGGFDSPTVAYSVL | |
| | VVADVEKGKAKKLKTVKELVGITIMERSSFEKDPVDFLESKGYQNIQKDNII | |
| | KLPKYSLFELENGRKRLLASAGELQKGNELALPAKYVTLLYHASHYEKLKES | |
| | PEDNEKKLRYVEEHREEFDEILDQIEEFSERYILADKNLEKILELYAKNENA | |
| | SISELASSFINLFTFTALGAPAAFKFFGSTIDRKRYTSTKEILNATLIHQSI | |
| | TGLYETRIDLSQLGGD | |
| CasEnd-177 | MDKKYSIGLDIGTNSVGWAVITDEYKVPSKKFKVLGNTDRHSIKKNLIGALL | 137 |
| | FDSGETAEATRLKRTARRRYTRRKNRICYLQEIFSNEMAKVDDSFFHRLEES | |
| | FLVEEDKRGERHPIFGNIVDEVAYHEKYPTIYHLRKHLADSTEKADLRLIYL | |
| | ALAHMIKFRGHFLIEGDLNSENSDVQKLFEQFVQVYDRTFEESHLSEETVDA | |
| | KAILTEKVSKSRRLENLIKQFPNEKKNGLFGNLIALSLGLQPNFKSNEDLSE | |
| | DAKLQFSKDTYDEDLENLLGQIGDQYADLFVAAKNLYDAILLSGILTVNDES | |
| | TKAPLSASMIKRYDEHHQDLTLLKAFVRKQLPEKYKEIFFDESKNGYAGYID | |
| | GGASQEEFYKYIKPILSKIDGSEYFLEKIEREDFLRKQRTFDNGSIPHQIHL | |
| | GELHAILRRQEEYYPFLKENQEKIEKILTFRIPYYVGPLARGNSRFAWMSRK | |
| | SDETITPWNFEEVVDKEASAQAFIERMTNNDKNLPEEKVLPKHSLLYEKFTV | |
| | YNELTKVKYVTEGMGKPEFFDAEQKQEIFDLLFKKNRKVTKKKLLEFLFKEF | |
| | EEFRIVDISGVEDAFNASLGTYHDLLKIIKDKEFLDNEENEDILEDIILTLT | |
| | LFEDREMIEKRLSKYADLFDKKVLKKLKRRRYTGWGRLSAKLINGIRDKQSG | |
| | KTILDYLIDDGFANRNFMQLIHDDSLSFKEEIKKAQVIGQSDSLHEHIANLA | |
| | GSPAIKKGILQTVKVVDELVKVMGRHKPENIVIEMARENQTTQKGQRNSRER | |
| | LKRLEEAIKELGSQILKEHPVDNTQLQNDKLYLYYLQNGKDMYTGEELDIDR | |
| | LSDYDVDHIIPQSFIKDDSIDNRVLTSSAKARGKSDDVPSEEVVKKMKSYWE | |
| | QLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSSFEKNPVAFLEAKGYKNIQEDTII | |
| | KLPKYSLFELENGRKRLLASAGELQKGNELALPQKYVTLLYHAKRYEKLKEK | |
| | EEDNEKHLEYVEQHREEFDEILDQISEFAERYILADKNLEKIQKLYEKNESY | |
| | SISELASSFINLLTFTALGAPAAFKFFGTTIDRKRYTSTKEILNATLIHQSI | |
| | TGLYETRIDLSKLGGD | |
| CasEnd-178 | MDKKYSIGLDIGTNSVGWAVITDEYKVPSKKFKVLGNTDRQSIKKNLLGALL | 138 |
| | FDSGETAEATRLKRTARRRYTRRKNRIRYLQEIFSEEMVKVDDSFFHRLEES | |
| | FLVEEDKKYERHPIFGNIVDEVAYHEKYPTIYHLRKKLADSTEKADLRLIYL | |
| | ALAHIIKFRGHFLIEGDLNSENSDVDKLFHQLVQTYNQLFEENPINEEGVDA | |
| | KAILSERLSKSRRLENLIAQFPGEKKNGLFGNLIALSLGLTPNFKSNEDLAE | |
| | DAKLQLSKDTYDEDLENLLAQIGDQYADLFLAAKNLSDAILLSGILTVNDES | |
| | TKAPLSASMIKRYDEHHQDLTLLKALVRKQLPEKYKEIFFDKSKNGYAGYID | |
| | GGASQEEFYKYIKPILEKMDGTEEFLAKIEREDFLRKQRTFDNGSIPHQIHL | |
| | GELHAILRRQEEFYPFLKENQEKIEKILTFRIPYYVGPLARGNSRFAWMTRK | |
| | SEETITPWNFEEVVDKEASAESFIERMTNFDKNLPNEKVLPKHSLLYEYFTV | |
| | YNELTKVKYVTEGMGKPEFFSAKQKQEIVDLLFKKNRKVTVKQLKEFLFKKI | |
| | ECFDIVEISGVEDSFNASLGTYHDLLKIIKDKDFLDNEENEDILEDIVLTLT | |
| | LFEDREMIEKRLSKYADLFDKKVLKQLKRRRYTGWGRLSRKLINGIRDKQSG | |
| | KTILDFLKSDGFANRNFMQLIHDDSLTFKEEIEKAQVKGQGDSLHEQIANLA | |
| | GSPAIKKGILQSVKIVDELVKVMGRHKPENIVIEMARENQTTQKGQKNSRER | |
| | MKRLEEGIKELGSQILKEHPVENTQLQNEKLYLYYLQNGKDMYTGDELDIDR | |
| | LSDYDVDHIIPQSFIKDDSIDNKVLTSSAKARGKSDNVPSEEVVKKMKSYWR | |
| | RLLKAKLISQRKFDNLTKAERGGLSELDKAGFIKRQLVETRQITKHVAQILD | |
| | SRFNTEFDENDKLIRDVKIITLKSKLVSDFRKDFQFYKVREINDYHHAHDAY | |
| | LNAVVGTALIKKYPKLESEFVYGDYKVYDSRKMIAKSEQEIGKATAKMFFYS | |
| | NIMNFFKSEIKLADGEIRKRPLIETNEETGEIVWDKEKDFATVRKVLSMPQV | |
| | NIVKKTEVQTGGFSKESILPKRKSDKLIARKKDWDPKKYGGFDSPTVAYSVL | |
| | VVAKIEKGKKKKLKTVKELVGITIMERSSFEKNPVAFLEAKGYKNVQKDLII | |
| | KLPKYSLFELENGRKRLLASAGELQKGNELALPQKYVTFLYLASHYEKLKGS | |
| | PEDEEIKKEYVEQHRHYFDEILEQISEFSERYILADKNLEKILSLYSKNRDL | |
| | SISEQAESFINLFTFTALGAPAAFKFFGGTIDRKRYTSTKEILNATLIHQSI | |
| | TGLYETRIDLSQLGGD | |
| CasEnd-179 | MKKPYSIGLDIGTNSVGWAVVTDEYKVPSKKFKVLGNTDRSSIKKNLLGALL | 139 |
| | FDSGETAEATRLKRTARRRYTRRKNRILYLQEIFAEEMNKVDDSFFHRLEES | |
| | FLVEEDKRGERHPIFGNIVEEVAYHEKYPTIYHLRKHLADSTEKADLRLVYL | |
| | ALAHIIKFRGHFLIEGDLNSENTDVQKLFKKFVEVYDRTFEESHLSEETVDA | |
| | ESILTEKLSKSRKLENLIKQFPNEKKNGLFGNLIALSLGLQPNFKSNFNLSE | |
| | DAKLQFSKDTYDEDLENLLGQIGDEYADLFVAAKNLYDAILLSGILTVNDES | |
| | TKAPLSASMIKRYDEHHQDLTLLKKFIRENLPEKYKEIFFDESKNGYAGYID | |
| | GGASQEEFYKYLKNLLSKIDGSEYFLDKIEREDFLRKQRTFDNGSIPHQIHL | |
| | QELHAILRRQGEYYPFLKENQEKIEKILTFRIPYYVGPLARGNSRFAWASRK | |
| | SDETITPWNFDEVIDKEASAEAFIERMTNNDKNLPNEKVLPKHSLLYEKFTV | |
| | YNELTKVKYVTEGMGKAEFFDANMKQEIFDGLFKKNRKVTKKKLLEFLDKEF | |
| | DEFRIVDISGVEKAFNASLGTYHDLLKIIKDKDFLDNEENEDILEDIVLTLT | |
| | LFEDREMIKKRLSKYADLFDKKVLKKLERRRYTGWGRLSAKLINGIRDKQSG | |
| | KTILDYLIDDGFANRNFMQLIHDDNLSFKEEIAKAQVIGDSDSLHEVIANLA | |
| | GSPAIKKGILQSLKIVDELVKVMGRHNPENIVIEMARENQTTQKGQRNSRER | |
| | LKRLEEAIKELGSNILKEHPVENTQLQNDKLYLYYLQNGKDMYTGEELDIDR | |
| | LSDYDVDHIIPQSFIKDDSIDNRVLTSSAKARGKSDDVPSEEVVKKMKSFWS | |
| | KLLDAKLISQRKFDNLTKAERGGLTELDKAGFIKRQLVETRQITKHVAQILD | |
| | ERFNTEFDENNKLIRKVKIITLKSKLVSNFRKEFELYKVREINDYHHAHDAY | |
| | LNAVVGKALIKKYPKLESEFVYGDYPVYNSYKLIAKSDQEIGKATAKMFFYS | |
| | NIMNFFKSDIKLADGTIIERPDIEVNEETGEIAWDKTKHIATVKKVLSMPQV | |
| | NIVKKTEVQTGGFSKESILPKRNSDKLIARKKDWDTKKYGGFDSPTVAYSVL | |
| | VVADIEKGKSKKLKTVKELVGITIMERSRFEKNPVAFLEDKGYQNIQEDNII | |
| | KLPKYSLFELENGRKRLLASAGELQKGNELALPNKYVTLLYHAKHYEKLKEK | |
| | KEDIEKHLEYVEEHRDEFDEILDQISEFSKRYILADKNLEKIEELYEKNEDA | |
| | SIEELASSFINLLTFTALGAPAAFKFFGKTIDRKRYTSTTEILNATLIHQSI | |
| | TGLYETRIDLSKLGGD | |
| CasEnd-180 | MDKPYSIGLDIGTNSVGWAVITDEYKVPSKKFKVLGNTDRHSIKKNLLGALL | 140 |
| | FDSGETAEATRLKRTARRRYTRRKNRIRYLQEIFAEEMSKVDDSFFHRLEES | |
| | FLVEEDKRGERHPIFGNIVDEVAYHEKYPTIYHLRKHLADSTEKADLRLVYL | |
| | ALAHIIKFRGHFLIEGDLDAENTDVQKLFKKFVQVYDKTFEESHLSEQTVDA | |
| | ESILTDKLSKSRKLENLIKLFPNEKKNGLFGNLIALSLGLQPNFKINFELSE | |
| | DAKLQFSKDTYEEDLENLLGQIGDEYADLFLAAKNLYDAILLSGILTVNTEI | |
| | TKAPLSASMVKRYDEHHQDLTKLKAFIREQLPEKYKEIFFDESKNGYAGYID | |
| | GGAKQEEFYKYLKNLLSKLDGSEYFLDKIEREDFLRKQRTFDNGSIPHQIHL | |
| | QELHAILRRQGEYYPFLKENQEKIEKILTFRIPYYVGPLARGNSRFAWASRK | |
| | SDETITPWNFEEVIDKEKSAEAFIERMTNFDKNLPEEKVLPKHSLLYEKFTV | |
| | YNELTKVKYITEGMRKPAFFDANQKQEIFDLLFKKNRKVTKKKLKEFLFKEF | |
| | DEFRIVDISGVEKAFNASLGTYHDLLKIIKDKEFLDNEENEDILEDIVLTLT | |
| | LFEDREMIEQRLSKYADLFDKKVLKKLERRRYTGWGRLSAKLINGIRDKQSG | |
| | KTILDYLIDDGFANRNFMQLIHDDSLSFKEEIQKAQVIGQSDSLHQVIAELA | |
| | GSPAIKKGILQSLKIVDELVKVMGRYNPENIVIEMARENQTTQKGQRNSRER | |
| | LKRLEESIKELGSKILKEHPVDNTQLQNDKLYLYYLQNGKDMYTGEELDIDR | |
| | LSDYDVDHIIPQSFIKDDSIDNRVLTSSAKARGKSDDVPSEEVVKKMKPFWN | |
| | QLLNAKLISQRKFDNLTKAERGGLSELDKAGFIKRQLVETRQITKHVAQILD | |
| | ERFNTKYDENDKLIRKVKIITLKSKLVSQFRKEFELYKVREINDYHHAHDAY | |
| | LNAVVGKALIKKYPKLESEFVYGDYPVYNSYKMIAKSEQEIGKATAKYFFYS | |
| | NIMNFFKTKVTLANGEIRKRPLIETNEETGEIVWDKEKDIATVRKVLSMPQV | |
| | NIVKKTEVQTGGFSKESILPKRDSDKLIARKKDWDTKKYGGFDSPTVAYSVL | |
| | VIADVEKGKTKKLKTVKELVGITIMERSRFEKNPVAFLEDKGYKNIQKDNII | |
| | KLPKYSLFELENGRKRLLASAGELQKGNELALPQKYVTLLYHAKHYEKLKES | |
| | PEDNEKHLEYVQKHRDEFDEILDQISEFSKRYILADKNLEKILELYSQNADA | |
| | DIEELASSFINLLTFTALGAPAAFKFFDKKIDRKRYTSTTEILNATLIHQSI | |
| | TGLYETRIDLSQLGGD | |
| CasEnd-181 | MDKPYSIGLDIGTNSVGWAVITDEYKVPSKKFKVLGNTDRHSIKKNLLGALL | 141 |
| | FDSGETAEATRLKRTARRRYTRRKNRICYLQEIFAEEMSKVDDSFFHRLEES | |
| | FLVEEDKRDERHPIFGNIVDEVAYHEKYPTIYHLRKKLADSTEKADLRLIYL | |
| | ALAHIIKFRGHFLIEGDLDSENTDVQKLFKALVQTYDQTFEESHLQEETVDA | |
| | ESILTAKISKSRRLENLIKQYPNEKKNGLFGNLIALSLGLQPNFKINFALAE | |
| | DAKLQFSKDTYDEDLENLLAQIGDEYADLFTAAKNLYDAILLSGILTVNTEI | |
| | TKAPLSASMIKRYDEHHQDLTLLKAFIREQLPEKYKEIFFDVTKNGYAGYID | |
| | GGASQEEFYKYLKNILEKVDGSEYFLDKIEREDFLRKQRTFDNGSIPHQIHL | |
| | QELKAILRRQEEYYPFLKENQEKIEKILTFRIPYYVGPLARGNSRFAWMSRK | |
| | SEETITPWNFEEVVDKESSAEAFIERMTNYDKNLPEEKVLPKHSLLYEEFTV | |
| | YNELTKVKYVTEGMRKPAFFDAEQKKEIVDGLFKKNRKVTKKKLKEFLFKEI | |
| | DCFRIVEISGVEKAFNASLGTYHDLLKIIKDKDFLDNEENEKILEDIVLTLT | |
| | LFEDREMIEQRLSKYADLFDKKVLKQLKRRRYTGWGRLSAKLINGIRDKQSG | |
| | KTILDYLIDDGFANRNFMQLIHDDSLSFKEEIQKAQVIGQGDSLHQTIAELA | |
| | GSPAIKKGILQSLKIVDELVKVMGRHNPENIVIEMARENQTTQKGQRNSRER | |
| | LKRLEESIKELGSQILKEHPVENTQLQNDKLYLYYLQNGKDMYTGEELDIDN | |
| | LSDYDVDHIIPQSFIKDDSIDNRVLTSSKKARGKSDNVPSEEVVKKMKSEWN | |
| | RLLDAKLISQRKFDNLTKAERGGLTELDKAGFIKRQLVETRQITKHVAQILD | |
| | SRFNTKYDENDKLIRTVKIITLKSKLVSNFRKEFGFYKVREINDYHHAHDAY | |
| | LNAVVGTALIKKYPKLESEFVYGDYKVYDSYKMIAKSEQEIGKATAKYFFYS | |
| | NIMNFFKTDITLANGEIRKRPLIETNEETGEIVWDKQKDFATVRKVLSMPQV | |
| | NIVKKTEVQTGGFSKESILPKRNSDKLIARKKDWDTKKYGGFDSPTVAYSVL | |
| | VVADIEKGKAKKLKTVKELVGITIMERSSFEKNPIAFLENKGYQNIQKDNII | |
| | KLPKYSLFELENGRKRLLASAGELQKGNELALPNKYVTLLYHAKNYEKLKGS | |
| | PEDNPKHLEYVEQHRSEFDEIFDQISEFSQRYILADKNLEKILELYEQNRER | |
| | DISELASSFINLLTFTALGAPAAFKFFGTTIDRKRYTSTTEILNATLIHQSI | |
| | TGLYETRIDLSQLGGD | |
| | MDKKYSIGLDIGTNSVGWAVVTDEYKVPSKKFKVLGNTDRKSIKKNLLGALL | |
| | FDSGETAEATRLKRTARRRYTRRKNRIRYLQEIFAEEMAKVDDSFFHRLEES | |
| | FLVEEDKRHERHPIFGNIVEEVAYHEKYPTIYHLRKKLADSDEKADLRLVYL | |
| | ALAHIIKFRGHFLIEGDLDSENTDVQKLFKELVETYNQLFEESPLEEEEVDA | |
| | EAILTEKLSKSRRLENLIAQFPGEKKNGLFGNLLALSLGLTPNFKSNEDLEE | |
| | DAKLQFSKDTYDEDLEELLGQIGDEYADLFLAAKNLYDAILLSGILTVKDES | |
| CasEnd-182 | TKAPLSASMVKRYDEHHQDLTLLKQFIRKQLPEKYKEIFFDQSKNGYAGYID | 142 |
| | GGASQEEFYKYLKKILEKIDGSEEFLDKIEREDFLRKQRTFDNGSIPHQIHL | |
| | QELHAILRRQEKYYPFLKENQEKIEKILTFRIPYYVGPLARGNSRFAWLSRK | |
| | SDETITPWNFDEVVDKEASAEAFIERMTNFDKNLPEEKVLPKHSLLYEKFTV | |
| | YNELTKVKYVTEGMRKPEFFDAEQKQEIVDLLFKENRKVTKKQLKEYLFKKF | |
| | ECFDIVEISGVEDRFNASLGTYHDLLKILFDKDFLDNEANEEILEDIILTLT | |
| | LFEDREMIKKRLEKYSDLFDKKQLKKLERRRYTGWGRLSKKLINGIRDKQTG | |
| | KTILDYLIDDGFANRNFMQLIHDDSLTFKEEIEKAQVIGDGESLHEVIANLA | |
| | GSPAIKKGILQSLKIVDEIVKVMGRHNPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFELYKVREINNYHHAHDAY | |
| | LNAVVGTALIKKYPKLESEFVYGDYKVYDVRKMIAKSEQEIGKATAKYFFYS | |
| | NIMNFFKTEVTLANGEIRKRPLIETNEETGEIVWDKEKDFATVRKVLSMPQV | |
| | NIVKKTEVQTGGFSKESILPKRDSDKLIARKKDWDTKKYGGFDSPTVAYSVL | |
| | VIADVEKGKAKKLKTVKELVGITIMERSAFEKDPIAFLEDKGYKNIKKDNII | |
| | KLPKYSLFELENGRKRLLASAGELQKGNELALPQHYVELLYHASHYEKLKGK | |
| | PEDNEEKQLYVEQHKDYFDEILDQISEFSKRYILADANLEKIKKLYEKNKDA | |
| | SIEELAENFIHLLTFTALGAPAAFKFFGKTIDRKRYTSTKEILNATLIHQSI | |
| | TGLYETRIDLSKLGGD | |
| CasEnd-183 | MKKPYSIGLDIGTNSVGWAVVTDEYKVPSKKFKVLGNTDRQSIKKNLLGALL | 143 |
| | FDSGETAEATRLKRTARRRYTRRKNRILYLQEIFAEEMNKVDDSFFHRLEES | |
| | FLVEEDKRGERHPIFGNIVEEVAYHEKYPTIYHLRKELADSDEKADLRLVYL | |
| | ALAHMIKFRGHFLIEGDLDSENTDVQKLFKQFLEAYDNTFEESHLSEETVDI | |
| | EEILTEKLSKSRKLENLLALFPNEKKNGIFGELLKLILGLQPNFKKNFGLSE | |
| | DAKLQFSKDTYDEDLEELLGQIGDEYADLFVAAKNLYDAILLSGILTVDDLS | |
| | TKAPLSASMVKRYDEHHQDLTLLKQFIRKQLPEKYKEIFFDKSKNGYAGYID | |
| | GGASQEDFYKYLKKLLEKIDGSEYFLDKIDREDFLRKQRTFDNGSIPHQIHL | |
| | EELKAIIRRQGEYYPFLKENQEKIEKILTFRIPYYVGPLARGNSRFAWASRK | |
| | SDETITPWNFDEVVDKEKSAEAFIERMTNNDKNLPTEKVLPKHSLLYEKFTV | |
| | YNELTKVKYVTEQMGKAKFFDANMKQEIFDGLFKKYRKVTKKKLLEFLDKEF | |
| | DEFRIVDISGVEKAFNASLGTYHDLLKIIKDKEFLDDEDNEKILEDIILTLT | |
| | LFEDREMIRKRLEKYADLFDKKQLKKLERRRYTGWGRLSAKLINGIRDKQTG | |
| | KTILDYLIDDGRSNRNFMQLIHDDSLSFKEEIAKAQLIGDSDSLHEVIADLA | |
| | GSPAIKKGILQSLKIVDELVKVMGRYNPENIVVEMARENQTTQKGQRNSRER | |
| | LKRLEEAIKNLGSDILKEYPVDNTQLQNDKLYLYYLQNGKDMYTGEELDIDN | |
| | LSDYDVDHIIPQSFIKDDSIDNRVLVSSKKARGKSDDVPSEDVVNKMKPFWK | |
| | KLLKAKLISQRKYDNLTKAERGGLTELDKAGFIKRQLVETRQITKHVAQILD | |
| | ERFNTEFDENNKLIRDVKIITLKSKLVSQFRKEFGLYKVREINDYHHAHDAY | |
| | LNAVVGKALIKKYPKLESEFVYGDYPVYNSYKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-184 | MKKPYSIGLDIGTNSVGWAVVTDDYKVPAKKMKVLGNTDKEYIKKNLLGALL | 144 |
| | FDSGNTAEDRRLKRTARRRYTRRRNRILYLQEIFSEEMNKVDESFFHRLEDS | |
| | FLVPEDKRGERHPIFGDLEEEVKYHEDFPTIYHLRKELADSPEKADLRLVYL | |
| | ALAHIIKYRGHFLIEGELDTRNNDIQKLFQEFLAVYDNTFENSSLSEQNVQV | |
| | EEILTDKISKSAKKDRVLKLFPNEKSNGLFAEFLKLIVGNQADFKKHFELEE | |
| | KAPLQFSKDSYDEDLEGLLGQIGDEYADLFLSAKKLYDAILLSGILTVTDVS | |
| | TKAPLSASMIQRYVEHQEDLKKLKQFIRTNLPAKYNEVENDKSKDGYAGYID | |
| | GKTNQEDFYKYLKKLLTKVAGSEYFLEKIEREDFLRKQRTFDNGSIPHQIHL | |
| | QEMKAIIRRQAEYYPFLKENQDKIEKILTFRIPYYVGPLARGNSDFAWASRK | |
| | SDEKITPWNEDDIIDKESSAEAFINRMTNYDLYLPEEKVLPKHSLLYEKFTV | |
| | YNELTKVRYITEQMGETEFFDANMKQEIFDGLFKKYRKVTKKKLLNFLEKEF | |
| | DEFRIVDITGLDKAFNASLGTYHDLLKILKDKEFLDDPANEEILEDIVQTLT | |
| | LFEDREMIKKRLSKYSDLFDKKQLKKLERRHYTGWGRLSAKLINGIRDKQTR | |
| | KTILDYLIDDGNSNRNFMQLINDDGLSFKEEIAKAQVIGESDNLKQVVQDLA | |
| | GSPAIKKGILQSLKIVDELVKIMGGYNPESIVVEMARENQFTNRGRRNSQQR | |
| | LKKLTDSIKELGSNILKEHPVDNSQLQNDRLFLYYLQNGKDMYTGEALDIDY | |
| | LSQYDIDHIIPQAFIKDDSLDNRVLVSSAKARGKSDDVPSKDVVKKMKSFWN | |
| | KLLDAKLISQRKFDNLTKAERGGLTDDDKAGFIKRQLVETRQITKHVARILD | |
| | ERFNTELDENNKKIRKVKIVTLKSNLVSNFRKEFELYKVREINDYHHAHDAY | |
| | LNAVVAKALLGKYPKLEPEFVYGEYPKYNSHKYVSQTDEERNTATAKKFFYS | |
| | NIMNFFKSDVKLADGSEVERPQIERNDETGEIVWDKTKHVEIVKKVLSYPQV | |
| | NIVKKVEEQTGGFSKESILPKGNSDKLIPRKTKWDTKKYGGFDSPTVAYSVL | |
| | VIADIEKGKSKKLKTVKELVGITIMEKKKFEKDPVAFLERKGYQNIQEENII | |
| | KLPKYSLFELENGRRRLLASAKELQKGNEIVLPNHLVKLLYHAKHIHSIDEK | |
| | NEEIKKHLQYVKKHRQEFSELLDEVKDFSKKYVLAEKNLEKIEELYAKNEQA | |
| | SVEELANSFINLLTFTAMGAPATFKFFGTTIDRKRYTSTTEILNATLIHQSI | |
| | TGLYETRIDLSKLGED | |
| CasEnd-185 | MKKPYSIGLDIGTNSVGWAVVTDDYKVPSKKMKVLGNTDRQSIKKNLLGALL | 145 |
| | FDSGETAEATRLKRTARRRYTRRKNRILYLQEIFSEEMSKVDESFFHRLEDS | |
| | FLVEEDKRNERHPIFGNIVDEVAYHEKFPTIYHLRKHLADSTEKADLRLVYL | |
| | ALAHIIKFRGHFLIEGDLDSENTDVQKLFKQFVQVYNKTFEESHLSEETVDA | |
| | EAILTEKLSKSRKLENLLAQFPNEKKNGLFGNLIALSLGLQPNFKSNFELSE | |
| | DAKLQFSKDTYDEDLDNLLGQIGDEYADVFVAAKNLYDAILLSGILTVNDLS | |
| | TKAPLSASMIKRYEEHHEDLTKLKNFVRKNLPEKYKEIFFDKSKNGYAGYID | |
| | GGTSQEEFYKYIKKNLSKVDGSEYFLEKIDREDFLRKQRTFDNGSIPHQIHL | |
| | GELHAILRRQEEYYPFLKENQEKIEKILTFRIPYYVGPLARGNSRFAWMTRK | |
| | SDETITPWNFDEVVDKEASAQAFIERMTNNDLYLPTEKVLPKHSLLYEKFTV | |
| | YNELTKVKYVTEGMGKTKFFDANQKKEIFDGLFKKNRKVTKKKLLNFLFKEF | |
| | DEFRIVDISGVEDAFNASLGTYHDLLKIIKDKEFLDNEENEKILEDIVLTLT | |
| | LFEDREMIKKRLLKYADLFDKKVLKKLERRHYTGWGRLSAKLINGIRDKQTG | |
| | KTILDFLIDDGFANRNFMQLIHDDNLSFKEEIQKAQVIGQEDSLHEVVAELA | |
| | GSPAIKKGILQSLKIVDELVKVMGRHAPENIVVEMARENQTTAKGQRNSRER | |
| | LKRLEEAMKELGSSILKEHPVENSQLQNDRLYLYYLQNGKDMYTGEELDIDR | |
| | LSDYDVDHIIPQSFIKDDSIDNRVLTSSAKARGKSDDVPSEEVVKKMKSFWY | |
| | QLLNAKLISQRKFDNLTKAERGGLTEDDKAGFIKRQLVETRQITKHVAQILD | |
| | ERFNTEFDENDKLIRDVKIVTLKSKLVSNFRKEFQFYKVREINDYHHAHDAY | |
| | LNAVVGKALIKKYPKLEPEFVYGDYPKYNSYKMFAKSEKERGKATAKMFFYS | |
| | NIMNFFKTDIKLADGQIIKRPQIETNEETGEIVWDKGKDFATVKKVLSMPQV | |
| | NIVKKTEVQTGGFSKESILPKRNSDKLIARKKGWDTKKYGGFDSPTVAYSVL | |
| | VVADIEKGKAKKLKTVKELVGITIMERSRFEKNPVAFLEDKGYQNIRKDSII | |
| | KLPKYSLFELENGRKRLLASAGELQKGNELALPNKLVTLLYHAKNLEKLDEK | |
| | DEDNPKHREYVNQHREEFKEIFQQISEFSKRYILADKNLEKILELYEKNENK | |
| | SISELASSFINLLTFTALGAPAAFKFFDKTIDRKRYTSTKEILNATLIHQSI | |
| | TGLYETRIDLSKLGGD | |
| CasEnd-186 | MKKPYSIGLDIGTNSVGWAVITDDYKVPSKKMKVLGNTDRSSIKKNLLGALL | 146 |
| | FDSGETAEATRLKRTARRRYTRRKNRIRYLQEIFAEEMNKVDDSFFHRLEDS | |
| | FLVEEDKRGERHPIFGNIVEEVAYHEKFPTIYHLRKYLADSTEKADLRLVYL | |
| | ALAHIIKFRGHFLIEGDLNPENTDVQKLFKKFVTTYDKTFEESHLSEETVDA | |
| | EAILTEKLSKSRKLENLLKQFPKEKKNGLFGNLIALSLGLQPNFKSNFQLSE | |
| | DAKLQFSKDTYDEDLENLLAQIGDEYADLFVAAKNLYDAILLSGILTVNDES | |
| | TKAPLSASMIKRYEEHHEDLTLLKAFIRKQLPEKYKEIFFDKSKNGYAGYID | |
| | GGTSQEEFYKYLKPLLEKLDGSEYFLDKIEREDFLRKQRTFDNGSIPHQIHL | |
| | KELHAILRRQAEYYPFLKENQEKIEKILTFRIPYYVGPLARGNSRFAWASRK | |
| | SDETITPWNFDEVIDKEASAEAFIERMTNFDLYLPEEKVLPKHSLLYEKFTV | |
| | YNELTKVKYVTEGMGESQFFDAELKQEIFDGLFKKYRKVTKKKLLEFLDKEF | |
| | EEFRIVDISGVEKRFNASLGTYHDLLKIIKDKEFLDNPENEDILEDIVLTLT | |
| | LFEDREMIEQRLQKYADLFDKKVLKKLERRHYTGWGRLSAKLINGIRDKQTG | |
| | KTILDYLIDDGYANRNFMQLIHDDSLTFKEEIAKAQVIGNSDSLHEVVANLA | |
| | GSPAIKKGILQSLKIVDELVKVMGRYAPENIVIEMARENQTTAKGQRNSRER | |
| | LKRLEEAIKKLGSNILKEHPVENAQLQNDRLYLYYLQNGKDMYTGEELDINN | |
| | LSDYDVDHIIPQSFIKDDSIDNRVLTSSKKARGKSDDVPSIEVVKKMKSFWS | |
| | KLLNAKLISQRKFDNLTKAERGGLTEDDKAGFIKRQLVETRQITKHVAQILD | |
| | SRFNTERDENNKLIRKVKIITLKSKLVSNFRKDFGLYKVREINDYHHAHDAY | |
| | LNAVVGTALIKKYPKLEPEFVYGDYPKYNSYKMIRKSESERGKATAKMFFYS | |
| | NIMNFFKTDIKLADGRIEERPVIEVNEETGEIVWDKGKHFATVKKVLSMPQV | |
| | NIVKKTEVQTGGFSKESILPKRDSDKLIARKKKWDPKKYGGFDSPTVAYSVL | |
| | VVADIEKGKSKKLKTVKELVGITIMERSRFEKNPIAFLESKGYKNIQEDNII | |
| | KLPKYSLFELENGRKRLLASAGELQKGNELALPNKLVTLLYHAKHIENLKEK | |
| | PEDNEKKLEYVEQHRSEFDEILDQISEFSKRYILADKNLEKIEELYHKNNSK | |
| | SIEELAESFINLFTFTALGAPAAFKFFGATIDRKRYTSTTECLNATLIHQSI | |
| | TGLYETRIDLSKLGGD | |
| CasEnd-187 | MDKKYSIGLDIGTNSVGWAVITDEYKVPSKKFKVLGNTDRHSIKKNLIGALL | 147 |
| | FDSGETAEATRLKRTARRRYTRRKNRICYLQEIFSNEMAKVDDSFFHRLEES | |
| | FLVEEDKRNERHPIFGNIVDEVAYHEKYPTIYHLRKKLADSDEKADLRLIYL | |
| | ALAHMIKFRGHFLIEGDLNSDNSDVQKLFKQLVQVYNQLFEESPLNEETVDA | |
| | KAILTEKLSKSRRLENLIAQFPGEKKNGLFGNLIALSLGLTPNFKSNFDLAE | |
| | DAKLQFSKDTYDEDLENLLGQIGDEYADLFLAAKNLYDAILLSGILTVNDES | |
| | TKAPLSASMVKRYDEHHQDLTLLKAFVRQQLPEKYKEIFFDQSKNGYAGYID | |
| | GGASQEEFYKYLKKILEKVDGSEEFLAKINREDFLRKQRTFDNGSIPHQIHL | |
| | GELHAILRRQEKYYPFLKENREKIEKILTFRIPYYVGPLARGNSRFAWMTRK | |
| | SDETITPWNFDEVVDKEASAQAFIERMTNFDKNLPDEKVLPKHSLLYEKFTV | |
| | YNELTKVKYVTEGMRKPEFFSAEQKQEIVDLLFKKNRKVTKKQLKEYLFKEF | |
| | ECFDIVEISGVEDRFNASLGTYHDLLKIIFDKDFLDNEENEKILEDIVLTLT | |
| | LFEDREMIKERLEKYADLFDDKQLKQLKRRRYTGWGRLSKKLINGIRDKQSG | |
| | KTILDYLIDDGFANRNFMQLIHDDSLTFKEEIEKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-188 | MDKPYSIGLDIGTNSVGWAVVTDEYKVPSKKFKVLGNTGRKSIKKNLWGVLL | 148 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEQIANLA | |
| | GSPAIKKGILQSIKIVDEIVKVMGRYAPENIVVEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLTELDKAGFIKRQLVETRQITKHVAQILD | |
| | ERFNAEYDDNGKLIRDTKIVTLKSKLVSNFRKDFELYKIREVNNYHHAHDAY | |
| | LNAVVGQALIKKYPKLESEFVYGDYPVYDVNKLIRKSNREIGKATEKMFFYS | |
| | NIMNFFKSDVKLADGDVRKRPIVEVNEETGEIVWDKNKHLATIKKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-189 | MKKKYSIGLDIGTNSVGWAVITDEYKVPSKKFKVLGNTDRQSIKKNLIGALL | 149 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNPENSDVDKLFIQLVQTYNQLFEENPINESGVDA | |
| | KAILSAKLSKSRRLENLIAQLPGEKKNGLFGNLIALSLGLTPNFKSNFDLSE | |
| | DAKLQLSKDTYDDDLENLLGQIGDQYADLFLAAKNLSDAILLSDILRVNTES | |
| | TKAPLSASMIKRYDEHHQDLTLLKALVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SDETITPWNFEEVVDKGASAQSFIERMTNFDKNLPNEKVLPKHSLLYEYFTV | |
| | YNELTKVKYVTEGMGKPEFLSGEQKKEIVDLLFKKDRKVTVKQLKEDYFKEI | |
| | ECFDSVEISGVEDRFNASLGTYHDLLKIIKDKDFLDNEENEDILEDIVLTLT | |
| | LFEDREMIEERLKKYANLEDDKVMKQLKRRRYTGWGRLSRKLINGIRDKQSG | |
| | KTILDFLKSDGFANRNFMQLIHDDSLTFKEAIQKAQVIGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGDELDINR | |
| | LSDYDVDHIVPQSFLKDDSIDNKVLTRSKKARGKSDNVPSEEVVKKMKNYWR | |
| | QLLKAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEIKLANGEIRKRPLIETNEETGEIVWDKERDFATVRKVLSMPQV | |
| | NIVKKTEVQTGGFSKESILPKRDSDKLIARKKDWDPKKYGGFDSPTVAYSVL | |
| | VVAKIEKGKSKKLKTVKELLGITIMERSSFEKDPVAFLEDKGYKNVKKDLII | |
| | KLPKYSLFELENGRKRMLASAGELQKGNELALPSKYVNFLYLASHYEKLKGS | |
| | PEDNEQKQLYVEQHKHYLDEIIDQISEFSERVILADKNLEKVLSAYNEIRDK | |
| | SIREQAENIIHLFTLTNLGAPAAFKYFDTTIDRKRYTSTKEVLDATLIHQSI | |
| | TGLYETRIDLSQLGGD | |
| CasEnd-190 | MKKPYSIGLDIGTNSVGWAVVTDEYKVPSKKFKVLGNTDRSSIKKNLLGALL | 150 |
| | FDSGETAEATRLKRTARRRYTRRKNRILYLQEIFAEEMNKVDDSFFHRLEES | |
| | FLVEEDKRGERHPIFGNIVEEVAYHEEYPTIYHLRKYLADSTEKADLRLVYL | |
| | ALAHIIKFRGHFLIEGDLDAENTDVQKLFYDFVQAYNNTFEESHLSEATVDA | |
| | SEILTEKISKSRRLENLLKNFPTEKKNGFFGNLVALSLGLQPNFKINFELSE | |
| | DAKLQFSKDTYDEDLENLLGQIGDEYADLFVAAKNLYDAILLSGILTVNDES | |
| | TKAPLSASMIKRYDEHHQDLTQLKKFIREKLPEKYKEIFFDESKNGYAGYID | |
| | GGASQEEFYKYLKNLLSKIDGSEYFLDKIEREDFLRKQRTFDNGSIPHQIHL | |
| | QELHAILRRQGEYYPFLKENQEKIEKILTFRIPYYVGPLARGNSRFAWASRK | |
| | SDETITPWNFDEVVDKESSAEAFIERMTNNDKNLPNEKVLPKHSLLYEKFTV | |
| | YNELTKVKYVTEQMGKAKFFDANQKQEIVDLLFKKERKVTKKKLLDFLFKEF | |
| | DEFRIVDISGVEKAFNASLGTYHDLLKILKDKDFLDNEENEDILEDIVLTLT | |
| | LFEDREMIKQRLQKYEDLFDKKQLKKLERRRYTGWGRLSAKLINGIRDKQTG | |
| | KTILDYLIDDGRANRNFMQLIHDDNLSFKEEIAKAQVIGESDSLHQVIADLA | |
| | GSPAIKKGILQSLKIVDELVKVMGRYNPENIVVEMARENQTTQKGQRNSRER | |
| | LKRLEESIKKLGSKILKEHPVDNTQLQNDKLYLYYLQNGKDMYTGEELDIDR | |
| | LSDYDVDHIIPQSFIKDDSIDNRVLTSSAKARGKSDDVPSIDVVKKMKPFWQ | |
| | KLLDAKLISQRKFDNLTKAERGGLTELDKAGFIKRQLVETRQITKHVAQILD | |
| | SRFNTEFDENNKLIRKVKIITLKSKLVSNFRKEFELYKVREINDYHHAHDAY | |
| | LNAVVGKALIKKYPKLESEFVYGDYPVYNSYKVMAESNSEIGKATEKMFFYS | |
| | NIMNFFKSEVKLADGQIFERPQIEVNEETGEIAWDKVKHIRTVRKVLSMPQV | |
| | NIVKKTEVQTGGFSKESILPKRDSDKLIARKKDWDTKKYGGFDSPTVAYSVL | |
| | VVADIEKGKSKKLKTVKELVGITIMERSRFEKNPVAFLESKGYQNIQKDNII | |
| | KLPKYSLFELENGRKRLLASAGELQKGNELALPQHYVTLLYHAKHYEKLKEK | |
| | SEDIPKHLEYVKNHKQEFKELLNQISEFSERYILADKNLEKIRELYAKNQDA | |
| | SVEELASSFINLLTFTALGAPAAFKFFGKNIDRKRYTSTTECLNATLIHQSI | |
| | TGLYETRIDLSKLGED | |
| CasEnd-191 | MKKKYSIGLDIGTNSVGWAVITDEYKVPSKKFKVLGNTDRQSIKKNLIGALL | 151 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSENSDVDKLFIQLVQTYNQLFEENPINESGVDA | |
| | KAILSAKLSKSRRLENLIAQFPGEKKNGLFGNLIALSLGLTPNFKSNFDLAE | |
| | DAKLQLSKDTYDDDLENLLAQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNFDKNLPTEKVLPKHSLLYEYFTV | |
| | YNELTKVKYVTEGMRKPEFLSGEQKKEIVDLLFKKNRKVTVKQLKEDYFKKI | |
| | ECFDSVEISGVEDRFNASLGTYHDLLKIIKDKEFLDNEENEDILEDIVLTLT | |
| | LFEDREMIEERLKKYANLFDKKVMKQLKRRRYTGWGRLSRKLINGIRDKQSG | |
| | KTILDFLKSDGFANRNFMQLIHDDSLTFKEEIKKAQVIGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGDELDINR | |
| | LSDYDVDHIVPQSFLKDDSIDNKVLTRSKEARGKSDNVPSEEVVKKMKSYWR | |
| | QLLNAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEIKLANGEIRKRPLIETNEETGEIVWDKERDFATVRKVLSMPQV | |
| | NIVKKTEVQTGGFSKESILPKRESDKLIARKKDWDPKKYGGFDSPTVAYSVL | |
| | VVAKVEKGKTKKLKTVKELLGITIMERSSFEKDPVAFLEAKGYKEVKKDLII | |
| | KLPKYSLFELENGRKRMLASAGELQKGNELALPSKYVNFLYLASHYEKLKGK | |
| | PEDNEKKQLYVEQHKHYLDEIIDQISEFSERVILADKNLDKVLSAYNNERDK | |
| | SIREQAENIIHLFTLTNLGAPAAFKYFGTTIDRKRYTSTKEVLDATLIHQSI | |
| | TGLYETRIDLSQLGGD | |
| CasEnd-192 | MDKKYSIGLDIGTNSVGWAVVTDEYKVPSKKFKVLGNTDRHSIKKNLIGALL | 152 |
| | FDSGETAEATRLKRTARRRYTRRKNRILYLQEIFAEEMAKVDDSFFHRLEES | |
| | FLVEEDKRNERHPIFGNIVDEVAYHEKYPTIYHLRKELADSPEKADLRLVYL | |
| | ALAHIIKFRGHFLIEGDLNAENSDVQKLFDQLVQTYNQLFEESPLEEEGVDA | |
| | KAILTEKLSKSRRLENLIAEFPGEKKNGLFGNLIALSLGLTPNFKSNEDLSE | |
| | DAKLQLSKDTYDEDLEELLGQIGDEYADLFLAAKNLYDAILLSGILTVSDEI | |
| | TKAPLSASMVKRYDEHHQDLTLLKDLIRKQLPEKYKEIFFDQSKNGYAGYID | |
| | GGASQEEFYKYLKKILEKIDGSEEFLDKIEREDFLRKQRTFDNGSIPHQIHL | |
| | QELHAILRRQEKYYPFLKENREKIEKILTFRIPYYVGPLARGNSRFAWMSRK | |
| | SDETITPWNFEEVVDKEASAQAFIERMTNFDKNLPNEKVLPKHSLLYEKFTV | |
| | YNELTKVKYVTEGMRKPEFLSAEQKQEIVDLLFKKNRKVTVKQLKEYLFKEF | |
| | ECFDIVEISGVEDRFNASLGTYHDLLKIIKDKDFLDNPENEEILEDIVLTLT | |
| | LFEDREMIKQRLKKYADLFDKKQLKKLSRRRYTGWGRLSKKLINGIRDKQSG | |
| | KTILDYLKDDGFANRNFMQLIHDDSLTFKEEIKKAQVIGQGESLHELIANLA | |
| | GSPAIKKGILQSLKIVDEIVKVMGRYEPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFQLYKVREINNYHHAHDAY | |
| | LNAVVGTALIKKYPKLESEFVYGDYKVYDVRKMIAKSEREIGKATAKYFFYS | |
| | NIMNFFKSEITLANGEIRKRPLIEVNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSAFEKNPIAFLEAKGYKEIKKDLII | |
| | KLPKYSLFELENGRKRMLASAGELQKGNELALPQKYVTLLYHASHYEKLKGS | |
| | PEDNEEKQLFVEQHKHYFDEILEQISEFSKRYILADANLEKIKKLYEKNRDL | |
| | SIEELAENFIHLLTFTALGAPAAFKFFDTTIDRKRYTSTKEILNATLIHQSI | |
| | TGLYETRIDLSQLGGD | |
| CasEnd-193 | MDKKYSIGLDIGTNSVGWAVITDEYKVPSKKFKVLGNTDRHSIKKNLIGALL | 153 |
| | FDSGETAEATRLKRTARRRYTRRKNRICYLQEIFSNEMAKVDDSFFHRLEES | |
| | FLVEEDKKHERHPIFGNIVEEVAYHEKYPTIYHLRKKLADSDEKADLRLIYL | |
| | ALAHMIKFRGHFLIEGDLNSENSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEQIANLA | |
| | GSPAIKKGILQTIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIRDVKIITLKSKLVSDFRKDFQLYKVREINNYHHAHDAY | |
| | LNAVVGTALIKKYPKLESEFVYGDYKVYDVRKMIAKSEQEIGKATAKYFFYS | |
| | NIMNFFKTDVTLANGEIRKRPLIEVNEETGEIVWDKEKDFATVKKVLSMPQV | |
| | NIVKKTEVQTGGFSKESILPKRDSDKLIARKKDWDTKKYGGEDSPTVAYSVL | |
| | VIAKVEKGKAKKLKTVKELVGITIMERSSFEKDPVAFLEAKGYKDIQKELII | |
| | KLPKYSLFELENGRKRLLASAGELQKGNELALPNKYVTLLYHAKHYEKLKGS | |
| | PEDNKYKQIYVEQHQEYFDEIIDQIIEFSKRYILADANLEKLKSLYEKNRDA | |
| | SIEELAENFIHLLTFTNLGAPAAFKFFDTTIDRKRYTSTKEILNATLIHQSI | |
| | TGLYETRIDLSQLGGD | |
| CasEnd-194 | MKKPYSIGLDIGTNSVGWAVVTDDYKVPSKKMKVLGNTDRQSIKKNLIGALL | 154 |
| | FDSGETAEATRLKRTARRRYTRRKNRIRYLQEIFAEEMNKVDDSFFHRLEES | |
| | FLVEEDKKGERHPIFGNIVDEVKYHEKFPTIYHLRKHLADSTEKADLRLVYL | |
| | ALAHIIKFRGHFLIEGDLNARNTDVQKLFEQFVQVYDDTFEESHLSEETVDA | |
| | KAILTEKVSKSRRLENLIAQFPGEKKNGLFGNLIALSLGLQPNFKSNFELSE | |
| | DAKLQFSKDTYDEDLENLLGQIGDGYADLFVAAKNLYDAILLSGILTVNDES | |
| | TKAPLSASMIKRYDEHHKDLTKLKEFIRKQLPEKYKEIFFDQTKNGYAGYID | |
| | GGTSQEEFYKYIKKLLSKMDGSEYFLDKIEREDFLRKQRTFDNGSIPHQIHL | |
| | GELHAILRRQGEYYPFLKENQEKIEKILTFRIPYYVGPLARGNSRFAWASRK | |
| | SDETITPWNFEEVVDKEASAQAFIERMTNNDLYLPDEKVLPKHSLLYEKFTV | |
| | YNELTKVKYVTEGMGKAEFFSANLKQEIFDGLFKKYRKVTKKKLLEFLFKEF | |
| | DEFRIVDISGVEKAFNASLGTYHDLLKIIKDKEFLDDEENEEILEDIVLTLT | |
| | LFEDREMIRQRLSKYADLFDKKVLKKLERRHYTGWGRLSAKLINGIRDKQSG | |
| | KTILDYLIDDGFANRNFMQLIHDDRLTFKEEIKKAQVIGDGDSLHEIVAELA | |
| | GSPAIKKGILQSLKIVDELVKVMGRYNPENIVIEMARENQTTAKGQRNSRER | |
| | LKRLEEAIKDLGSNILKEHPVENTQLQNDRLYLYYLQNGKDMYTGEELDIDR | |
| | LSQYDVDHIIPQSFIKDDSIDNRVLTSSAKARGKSDNVPSIEVVKKMKSYWN | |
| | RLLNAKLISQRKFDNLTKAERGGLSEDDKAGFIKRQLVETRQITKHVAQILD | |
| | SRFNTEFDENNKLIRDVKIITLKSKLVSQFRKDFELYKVREINDYHHAHDAY | |
| | LNAVVGKALIKKYPKLEPEFVYGDYPKYNSYKMVGKSKQERGKATAKVFFYS | |
| | NIMNFFKSDVKLADGRIVERPVIETNEETGEIVWDKVKDIATVRKVLSMPQV | |
| | NIVKKTEVQTGGFSKESILPKRNSDKLIARKKKWDPKKYGGFDSPTVAYSVL | |
| | VVADIEKGKAKKLKTVKELVGITIMERSAFEKDPVAFLEDKGYQNIQKDLII | |
| | KLPKYSLFELENGRKRLLASAGELQKGNELALPNKLVTLLYHAKHIEKLTGK | |
| | PEDQEKKLQYVEEHKHDFDEILSQISEFSKRYILADKNLEKIEELYHKNRDA | |
| | SIEELASSFINLFTFTALGAPAAFKFLGTTIDRKRYTSTTECLNATLIHQSI | |
| | TGLYETRIDLSKLGGD | |
| CasEnd-195 | MKKPYSIGLDIGTNSVGWAVITDEYKVPSKKFKVLGNTDRQSIKKNLIGALL | 155 |
| | FDSGETAEATRLKRTARRRYTRRKNRICYLQEIFSEEMSKVDDSFFHRLEES | |
| | FLVEEDKRHERHPIFGNIVDEVAYHENYPTIYHLRKKLADSTEKADLRLIYL | |
| | ALAHMIKFRGHFLIEGDLNSENTDVQKLFIQLVQTYNQLFEENPIEEEGVDA | |
| | KAILSAKLSKSRRLENLIALLPGEKKNGLFGNLIALSLGLTPNFKSNFELAE | |
| | DAKLQLSKDTYDEDLENLLAQIGDQYADLFLAAKNLSDAILLSDILTVNDVI | |
| | TKAPLSASMIKRYDEHHQDLTLLKALVRQQLPEKYKEIFFDDSKNGYAGYID | |
| | GGASQEEFYKYIKPILEKMDGTEYLLAKIEREDFLRKQRTFDNGSIPHQIHL | |
| | GELHAILRRQEDFYPFLKENREKIEKILTFRIPYYVGPLARGNSRFAWLKRK | |
| | SEETITPWNFEEVVDKEASAQAFIERMTNFDKNLPNEKVLPKHSLLYEYFTV | |
| | YNELTKVKYVTEGMGKPEFLSGEQKEEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIEERLKKYAHLFDKKVLKKLKRRRYTGWGRLSRKLINGIRDKQSG | |
| | KTILDFLKSDGFANRNFMQLIHDDSLTFKEEIKKAQVKGQGESLHEVIADLA | |
| | GSPAIKKGILQSVKIVDELVKVMGRHNPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIREVKIITLKSKLVSDFRKDFGFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKERDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKNPIAFLEKKGYKNVQKELII | |
| | KLPKYSLFELENGRKRLLASAGELQKGNELALPSHYVTLLYLAKNYEKLKGK | |
| | IEDLSKHLEYVEQHKEYFDEIFDQIIEFSERYILADKNLSKILELYDENREK | |
| | DIKELAENFIHLLTFTSLGAPAAFKFFDTTIDRKRYTSTTEILNATLIHQSI | |
| | TGLYETRIDLSQLGGD | |
| CasEnd-196 | MDKKYSIGLDIGTNSVGWAVITDEYKVPSKKFKVLGNTDRHSIKKNLIGALL | 156 |
| | FDSGETAEATRLKRTARRRYTRRKNRICYLQEIFSNEMAKVDPSFFHRLEES | |
| | FLVEEDKRGERHPIFGNIVEEVAYHEKYPTIYHLRKHLADSDEKADLRLVYL | |
| | ALAHIIKFRGHFLIEGDLNAENSDVQKLFKELVQIYDQTFEESHLSEETVDA | |
| | EAILTEKLSKSRRLENLIAQFPGEKKNGLFGNLIKLSLGLTPNFKSNEDLEE | |
| | DAKLQFSKDTYDEDLEELLGQIGDEYADLFVAAKNLYDAILLSGILTVSDSS | |
| | TKAPLSASMVKRYDEHHQDLTLLKEFIREQLPEKYKEIFFDQSKNGYAGYID | |
| | GGASQEEFYKYLKKILEKIDGSEEFLDKIEREDFLRKQRTFDNGSIPHQIHL | |
| | QELHAIIRRQEKYYPFLKENKEKIEKILTFRIPYYVGPLARGNSRFAWLSRK | |
| | SDETITPWNFDEVVDKEASAEAFIERMTNFDKNLPDEKVLPKHSLLYEKFTV | |
| | YNELTKVKYVTEGGKKPEFFDANQKQEIVDNLFKKYRKVTKKQLLEYLEKEF | |
| | DEFRIVEISGVEDRFNASLGTYHDLLKIIFDKDFLDNEENEEILEDIVLTLT | |
| | LFEDREMIKKRLEKYADLFDKKQIKKLSRRRYTGWGRLSAKLINGIRDKQTG | |
| | KTILDYLIDDGFANRNFMQLIHDDSLSFKEEIEKAQVIGDGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKNPIAFLESKGYQNIQKDNII | |
| | KLPKYSLFELENGRKRLLASAGELQKGNELALPNHYVTLLYHAKHYEKLKGS | |
| | PEDNEEHQIYVEQHRDEFDEILDQISEFSKRYILADANIEKLKKLYEKNRDA | |
| | SIEELAENFIHLLTFTALGAPAAFKFFGKNIDRKRYTSTKEILNATLIHQSI | |
| | TGLYETRIDLSKLGGD | |
| CasEnd-197 | MDKKYSIGLDIGTNSVGWAVITDEYKVPSKKFKVLGNTDRHSIKKNLIGALL | 157 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTSKYGGFDSPTVAYSVL | |
| | VIAKVEKGKSKKLKTVKELVGITIMERSAFEKDPVAFLENKGYQNVQKELII | |
| | KLPKYSLFELENGRKRLLASAGELQKGNELALPNHYVTLLYHAKNYEKIKGS | |
| | EEDEKRKQIYVEDHRYEFDEILDQVSEFSERYILADANLEKITNLYEKNIEA | |
| | SIEELASSFLNLLKFTKLGAPAAFKFFGTTIDRKRYTSTKEILNATLIHQSI | |
| | TGLYETRIDLSKLGGD | |
| CasEnd-198 | MDKKYSIGLDIGTNSVGWAVITDEYKVPSKKFKVLGNTDRHSIKKNLIGALL | 158 |
| | FDSGETAEATRLKRTARRRYTRRKNRICYLQEIFSNEMAKVDPSFFERLEES | |
| | FLVEEDKKTSRHPIFGNIVEEVAYHEKYPTIYHLRKKLVDSDEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEE | |
| | TKAPLSASMIKRYDDHHQDLTKLKELVRKELPEKYKTIFFNQNANGYAGYID | |
| | GGATQEEFYAAIKPILESMSGTKDLLEKLDNRDLLRKQRTFDNGSIPHQIHL | |
| | GELRAILERQEKFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKKEVGEKKKLKKVKELLGITIMERSKFEKDPLKFLEEKGYKDVKEDEII | |
| | KLPKYSLFELGNGRKRMLASAGELQKGNELALPSEYVNFLYLASDYEKLKGD | |
| | PEEKEKKQKYVEENKQYLDDIINQISEFSKRVIKADANLEKVLKAYEKHKDK | |
| | PIKEQAENIIHLFTLTRLGAPAAFKYFDEKIDRKRYTSTKEVLDATLIHQSI | |
| | TGLYETRIDLKFLGGD | |
| CasEnd-199 | MDKKYSIGLDIGTNSVGWAVVTDEYKVPSKKFKVLGNTDRHSIKKNLLGALL | 159 |
| | FDSGETAEATRLKRTARRRYTRRKNRIRYLQEIFAEEMAKVDDSFFHRLEES | |
| | FLVEEDKRNERHPIFGNIVEEVAYHEKYPTIYHLRKKLADSTEKADLRLVYL | |
| | ALAHMIKFRGHFLIEGDLNSENSDVQKLFEQLVQTYNQLFEESPLEEEKVDA | |
| | KAILTEKLSKSRRLENLIANFPGEKKNGLFGNLIALSLGLTPNFKSNEDLSE | |
| | DAKLQLSKDTYDEDLENLLAQIGDQYADLFVAAKNLSDAILLSGILTVNDES | |
| | TKAPLSASMVKRYDEHHQDLTLLKKFVRDQLPEKYKEIFFDQSKNGYAGYID | |
| | GGASQEEFYKYIKPILEKVDGSEEFLDKIEREDFLRKQRTFDNGSIPHQIHL | |
| | QELHAILRRQEEYYPFLKENQEKIEKILTFRIPYYVGPLARGNSRFAWMSRK | |
| | SDETITPWNFEEVVDKEASAQKFIERMTNFDKNLPNEKVLPKHSLLYEKFTV | |
| | YNELTKVKYVTEGMRKPEFLSGEQKQEIVDLLFKKERKVTVKQLKEYLFKEF | |
| | DCFDSVEISGVEDRFNASLGTYHDLLKIIKDKDFLDNEENEDILEDIILTLT | |
| | LFEDREMIKKRLEKYADLFDKKVLKQLKRRRYTGWGRLSAKLINGIRDKQSG | |
| | KTILDYLIDDGFANRNFMQLIHDDSLTFKEEIEKAQVIGKGDSLHEQIANLA | |
| | GSPAIKKGILQTLKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENDKLIRDVKIITLKSKLVSDFRKDFELYKVREINNYHHAHDAY | |
| | LNAVVGTALIKKYPKLESEFVYGDYKVYDVRKMIAKSEQEIGKATAKYFFYS | |
| | NIMNFFKTDITLANGEIRKRPLIETNEETGEIVWDKEKDFATVRKVLSMPQV | |
| | NIVKKTEVQTGGFSKESILPKRDSDKLIARKKDWDTKKYGGFDSPTVAYSVL | |
| | VIAKVEKGKAKKLKTVKELVGITIMERSAFEKDPVAFLEAKGYQNIKKDLII | |
| | KLPKYSLFELENGRKRLLASAGELQKGNELALPQKYVTLLYHAKHYEKLKGK | |
| | PEDNEEKQLYVEQHKYYFDEIFDQISEFSKRYILADANLEKLISLYEKNRDK | |
| | SIEELAENFIHLLTFTALGAPAAFKFFDTTIDRKRYTSTKEILNATLIHQSI | |
| | TGLYETRIDLSKLGGD | |
| CasEnd-200 | MKKKYSIGLDIGTNSVGWAVITDEYKVPSKKFKVLGNTDRQSIKKNLIGALL | 160 |
| | FDSGETAEATRLKRTARRRYTRRKNRIRYLQEIFAEEMNKVDDSFFHRLEES | |
| | FLVEEDKRHDRHPIFGNIVDEVAYHENYPTIYHLRKELVDSPEKADLRLIYL | |
| | ALAHMIKFRGHFLIEGDLNSENTDVQKLFKQLVQTYNQLFEEKPLNEETVDA | |
| | EAILSEKLSKSRRLENLIAQFPGEKKNGLFGNLIALSLGLTPNFKSNFELSE | |
| | DAKLQLSKDTYDEDLENLLGQIGDEYADLFLAAKNLYDAILLSGILRVKDES | |
| | TKAPLSASMIKRYDEHHQDLTLLKALVRQQLPEKYKEIFFDASKNGYAGYID | |
| | GGASQEEFYKFIKPILSKMDGTEYLLDKLEREDLLRKQRTFDNGSIPHQIHL | |
| | QELHAILRRQEEYYPFLKENQEKIEKILTFRIPYYVGPLARGNSRFAWMTRK | |
| | SDETITPWNFEEVVDKGASAQAFIERMINNDKNLPNEKVLPKHSLLYEKFTV | |
| | YNELTKVKYVTEGMGKPEFLSGEQKKEIVDLLFKKERKVTVKKLKEFYFKEF | |
| | EEFRIVEISGVEDRFNASLGTYHDLLKIIKDKDFLDNEENEDILEDIVLTLT | |
| | LFEDREMIEKRLEKYANLFDKKVMKQLKRRRYTGWGRLSAKLINGIRDKQSG | |
| | KTILDFLISDGFANRNFMQLIHDDSLTFKEEIKKAQEIGQGDSLHEQIADLA | |
| | GSPAIKKGILQTVKVVDELVKVMGRHKPENIVIEMARENQTTQKGQKNSRER | |
| | LKRIEEGIKKLGSDILKEYPVENTQLQNDKLYLYYLQNGRDMYTGEELDIDR | |
| | LSDYDVDHIVPQSFIKDDSIDNKVLTRSKEARGKSDDVPSEDVVKKMKSYWR | |
| | QLLKAKLITQRKFDNLTKAERGGLTELDKAGFIKRQLVETRQITKHVAQILD | |
| | SRMNTEYDENNKLIRDVKVITLKSKLVSDFRKDFQFYKVREINDYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-201 | MDKKYSIGLDIGTNSVGWAVITDEYKVPSKKFKVLGNTDRKSIKKNLIGALL | 161 |
| | FDSGETAEATRLKRTARRRYTRRKNRICYLQEIFSSEMAKVDDSFFHRLEES | |
| | FLVEEDKRHERHPIFGNIVEEVAYHEKYPTIYHLRKKLVDSDDKADLRLIYL | |
| | ALAHMIKFRGHFLIEGDLNPDNSDVDKLFIQLLQTYNQLFEENPIEAEDIDA | |
| | KAILTERLSKSRRLENLIAQLPNEKKNGLFGNLIALSLGLTPNFKSNEDLAE | |
| | DAKLQLSKDTYDEDLENLLGQIGDQYADLFLAAKNLYDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLEREDLLRKQRTFDNGSIPHQIHL | |
| | GELHAILRRQEDFYPFLKDNREKIEKILTFRIPYYVGPLARGNSRFAWMTRK | |
| | SDETITPWNFEEVVDKGASAQSFIERMTNFDKNLPNEKVLPKHSLLYEYFTV | |
| | YNELTKVKYVTEGMRKPAFLDGEQKKEIVDLLFKTNRKVTVKQLKEDFFKKI | |
| | DCFDSVEISGVEDRFNASLGTYHDLLKIIKDKDELDNEENEDILEDIVLTLT | |
| | LFEDREMIEKRLKTYANLFDKKVMKQLKRRRYTGWGRLSRKLINGIRDKQSG | |
| | KTILDFLKSDGFANRNFMQLIHDDSLTFKEDIQKAQVSGQGDSLHEQIANLA | |
| | GSPAIKKGILQTLKVVDELVKVMGRYKPENIVIEMARENQTTQKGQKNSRER | |
| | MKRIEEGIKELGSDILKEHPVENTQLQNEKLYLYYLQNGRDMYTDQELDINR | |
| | LSDYDVDHIVPQSFLKDDSIDNKVLTSSEKARGKSDDVPSEEVVKKMKPYWR | |
| | QLLNAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKVYDVKKMIAKSDREIGKATAKYFFYS | |
| | NIMNFFKSDVKLADGTIRKRPLIEVNEETGEIVWDKGRDFATVRKVLSMPQV | |
| | NIVKKTEVQTGGFSKESILPKRNSDKLIARKKDWDPSKYGGEDSPTVAYSVL | |
| | VIAKIEKGKAKKLKSVKELLGITIMERSSFEKNPVDFLEAKGYQNIQKELII | |
| | KLPKYSLFELENGRKRLLASAGELQKGNELALPNKYVDFLYHASHYEKLKGS | |
| | PEDEKYSQLFVEQHRHYFDELFEQIIEFSERYILADANLEKIKNLYEKHSEL | |
| | SIREQAENILNLFTFTNLGAPAAFKYFDTDIDRKRYTSTKEILNATLIHQSI | |
| | TGLYETRIDLSQLGGD | |
| CasEnd-202 | MKKSYSIGLDIGTNSVGWAVVTDDYKVPSKKMKVLGNTDRSSIKKNLLGALL | 162 |
| | FDSGETAEATRLKRTARRRYTRRKNRILYLQEIFAEEMNKVDDSFFHRLEDS | |
| | FLVEEDKRNERHPIFGNIVEEVAYHEEFPTIYHLRKHLADSTEKADLRLVYL | |
| | ALAHMIKFRGHFLIEGDLNAENSDVQKLENDFVQHYNQTFEESPLSEETVDA | |
| | ESILTDKVSKSRKLENLIKQFPGEKKNGLFGNLIALSLGLQPNFKINFELSE | |
| | DAKLQFSKDTYDEDLENLLGQIGDEYADLFVAAKNLYDAILLSGILTVNDDS | |
| | TKAPLSASMIKRYEEHHEDLTLLKAFVRKNLPEKYKEIFFDKSKNGYAGYID | |
| | GGTSQEEFYKYLKKILEKVDGSEYFLDKIEREDFLRKQRTFDNGSIPHQIHL | |
| | QELKAILRRQETYYPFLKENQEKIEKILTFRIPYYVGPLARGNSRFAWMTRK | |
| | SDETITPWNFEEVVDKEASAEKFIERMINNDLYLPNEKVLPKHSLLYEKFTV | |
| | YNELTKVKYVTEGMGKAEFFDAEQKQEIVDLLFKKERKVTKKKLLDFLKKVF | |
| | DEFRIVDISGVEDAFNASLGTYHDLLKIIKDKEFLDDEENEDILEDIVLTLT | |
| | LFEDREMIKQRLSKYADLFDKKVLKKLERRHYTGWGRLSAKLINGIRDKQSG | |
| | KTILDYLIDDGFSNRNFMQLIHDDSLTFKEIIKKAQVSGNSDSLHEVVAELA | |
| | GSPAIKKGILQSLKIVDELVKVMGRYNPENIVVEMARENQTTNKGQRNSRER | |
| | LKRLEEAIKELGSKILKEHPVENTQLQNDRLYLYYLQNGKDMYTGEELDIDR | |
| | LSDYDVDHIIPQSFIKDDSIDNRVLTSSAKARGKSDDVPSEEVVKKMKSFWK | |
| | KLLRSKLISQRKFDNLTKAERGGLTEDDKAGFIKRQLVETRQITKHVAQILD | |
| | SRFNTEFDENNKLIRDVKIITLKSKLVSRFRKEFEFYKVREINDYHHAHDAY | |
| | LNAVVGTALIKKYPKLEPEFVYGDYPKYNSYKMIAKSEKERGKATAKMFFYS | |
| | NIMNFFKTDIKLADGRIRERPVIEVNEETGEIVWDKNKHIATVRKVLSMPQV | |
| | NIVKKTEVQTGGFSKESILPKRKSDKLIARKKKWDTKKYGGFDSPTVAYSVL | |
| | VVADIEKGKKKKLKTVKELVGITIMERSSFEKDPVAFLEDKGYQNIRKDNII | |
| | KLPKYSLFELENGRKRLLASAGELQKGNELALPQKLVTLLYHAKHIEKLTEK | |
| | KEDNEQHREYVEEHKHEFKEILDQISEFSKRYILADKNLEKILELYSKNREA | |
| | PIKELAESFINLFTFTALGAPADFKFFGTTIDRKRYTSTKEILNATLIHQSI | |
| | TGLYETRIDLSKLGGD | |
| CasEnd-203 | MDKPYSIGLDIGTNSVGWAVITDEYKVPSKKFKVLGNTDRHSIKKNLLGALL | 163 |
| | FDSGETAEATRLKRTARRRYTRRKNRIRYLQEIFAEEMNKVDDSFFHRLEES | |
| | FLVEEDKRSERHPIFGNIVDEVAYHEKYPTIYHLRKHLADSTEKADLRLVYL | |
| | ALAHIIKFRGHFLIEGDLNAENTDVQKLFIQFVQVYDNTFEESHLLESTVDA | |
| | EAILTAKISKSRRLENLINQFPNEKKNGLFGNLIALSLGLTPNFKTNFELSE | |
| | DAKLQFSKDTYEEDLENLLAQIGDQYADLFLAAKNLYDAILLSGILTVNTEI | |
| | TKAPLSASMIKRYDEHHQDLILLKAFIRNELPEKYKEIFFDESKNGYAGYID | |
| | GGAKQEEFYKYIKGILSKIEGAEYFLDKIEREDFLRKQRTFDNGSIPHQIHL | |
| | QELHAIIRRQEEFYPFLKENQEKIEKILTFRIPYYVGPLARGNSRFAWASRK | |
| | SDETITPWNFEEVVDKEKSAEAFIERMTNYDKNLPNEKVLPKHSLLYEKFTV | |
| | YNELTKVKYVTEGMRKPAFLDSNQKQEIVDGLFKENRKVTVKKLLNYLFKEF | |
| | EEFRIVEISGVEKAFNASLGTYHDLLKIIKDKEFLDNEENEKILEDIVLTLT | |
| | LFEDREMIKKRLKKYAHLFDKKVLKKLERRRYTGWGRLSAKLINGIRDKQTG | |
| | KTILDYLIDDGFANRNFMQLIHDDSLSFKEEIAKAQVIGETDSLHEVIADLA | |
| | GSPAIKKGILQSVKIVDELVKVMGRHNPENIVIEMARENQTTQKGQRNSRER | |
| | LKRLEESIKKLGSKILKEHPVENTQLQNDKLYLYYLQNGKDMYTGEELDIDH | |
| | LSDYDVDHIIPQSFIKDDSIDNRVLTSSKEARGKSDDVPSIEVVRKMKSFWS | |
| | KLLKAKLISQRKFDNLTKAERGGLTELDKAGFIKRQLVETRQITKHVAQILD | |
| | SRFNTKYDENDKLIRDVKIITLKSKLVSDFRKEFQLYKVREINDYHHAHDAY | |
| | LNAVVGTALIKKYPKLESEFVYGDYKVYNSHKMIAKSEQEIGKATAKYFFYS | |
| | NIMNFFKTDVTLANGEIRKRPLIETNEETGEIVWNKEKHFATVRKVLSMPQV | |
| | NIVKKTEVQTGGFSKESILPKRNSDKLIARKKDWDPKKYGGFDSPTVAYSVL | |
| | VVADIEKGKAKKLKTVKELVGITIMERSSFEKDPVAFLENKGYKNIQKDLII | |
| | KLPKYSLFELENGRKRLLASAGELQKGNELALPQKYVTLLYHAKHYEKLKGS | |
| | PEDNPKHLEYVKQHRDEFDEILDQIEEFAERYILADKNLEKIKELYEENRDA | |
| | DIKELAESFINLLTFTALGAPAAFKFFDKKIDRKRYTSTTEILNATLIHQSI | |
| | TGLYETRIDLSQLGGD | |
| CasEnd-204 | MKKPYSIGLDIGTNSVGWAVITDDYKVPSKKMKVLGNTDRQSIKKNLLGALL | 164 |
| | FDSGETAEATRLKRTARRRYTRRKNRILYLQEIFAEEMAKVDDSFFHRLEDS | |
| | FLVEEDKRGERHPIFGNIVEEVAYHEKFPTIYHLRKHLADSPEKADLRLVYL | |
| | ALAHIIKFRGHFLIEGDLDSENTDVQKLFKQFVEAYNNTFEESHLNEETVDA | |
| | EAILTEKISKSRRLENLIALFPTEKKNGLFGNLIKLSLGLQPNFKTNFGLSE | |
| | DAKLQFSKDTYEEDLENLLGQIGDEYADVFLAAKNLYDAILLSGILTVTDES | |
| | TKAPLSASMIKRYDEHHQDLTLLKAFVREQLPEKYKEIFFDESKNGYAGYID | |
| | GGTSQEEFYKYLKKILEKLDGSEYFLDKIEREDFLRKQRTFDNGSIPHQIHL | |
| | GELHAILRRQEEYYPFLKENQEKIEKILTFRIPYYVGPLARGNSRFAWASRK | |
| | SDETITPWNFEEVVDKEKSAQAFIERMTNNDLYLPEEKVLPKHSLLYEKFTV | |
| | YNELTKVKYVTEGMGEPEFFDANMKQEIVDELFKKNRKVTKKKLLEFLFKEF | |
| | DEFRIVDISGVEKAFNASLGTYHDLLKIIKDKDFLDDEENEDILEDIVLTLT | |
| | LFEDREMIEQRLQKYADLFDKKQLKKLKRRHYTGWGRLSAKLINGIRDKQSG | |
| | KTILDYLKDDGQANRNFMQLIHDDSLSFKEEIAKAQVIGQSDSLHEVVADLA | |
| | GSPAIKKGILQSLKIVDELVKVMGRHNPENIVIEMARENQTTAKGQRNSRER | |
| | LKGLEEAIKNLGSKILKEHPVENSQLQNDRLYLYYLQNGKDMYTGEELDIDR | |
| | LSQYDVDHIIPQSFIKDDSIDNRVLVSSKKARGKSDDVPSEEVVRKMKPYWR | |
| | KLLNAKLISQRKFDNLTKAERGGLTEDDKAGFIKRQLVETRQITKHVAQILD | |
| | SRFNKETDENDKLIRKVKIITLKSKLVSDFRKEFGFYKVREINDYHHAHDAY | |
| | LNAVVGKALIKKYPKLEPEFVYGDYPKYNSYKMIAKSDQEEGKATAKMFFYS | |
| | NLMNFFKTEIKLADGFIIERPQIEVNEETGEIVWDKTKHIATVRKVLSMPQV | |
| | NIVKKTEVQTGGFSKESILPKRNSDKLIARKKKWDTKKYGGFDSPTVAYSVL | |
| | VVADIEKGKAKKLKTVKELVGITIMERSTFEKNPIDFLEDKGYKNIQTDKII | |
| | KLPKYSLFELENGRKRLLASAGELQKGNELALPNKLVTLLYHAKHIEKLKEK | |
| | YEDNEEHKEYVEQHRSQFDEILEQIVEFSKRYILADKNLEKITSLYKENEDY | |
| | SVSELAESFINLLTFTALGAPAAFKFFGTDIDRKRYTSTKEILDATLIHQSI | |
| | TGLYETRIDLSKLGGD | |
| CasEnd-205 | MDKKYSIGLDIGTNSVGWAVITDEYKVPSKKFKVLGNTDRHSIKKNLIGALL | 165 |
| | FDSGETAEATRLKRTARRRYTRRKNRICYLQEIFSNEMAKVDESFFHRLEES | |
| | FLVEEDKRGERHPIFGNIVEEVAYHEKYPTIYHLRKHLADSPEKADLRLIYL | |
| | ALAHIIKFRGHFLIEGDLNTENSDVQKLFHAFVEVYDRLFEESHLNEETVDA | |
| | KAILTEKVSKSRRLENLIKQFPTEKKNGIFGNLIALSLGLQPNFKSNFGLSE | |
| | DAKLQFSKDTYEEDLENLLGQIGDEYADLFSAAKNLYDAILLSGILTVNDNI | |
| | TKAPLSASMIKRYDEHHQDLTLLKAFVRQQLPEKYKEIFFDETKNGYAGYID | |
| | GGASQEEFYKYIKPILKKIDGSEYFLDKIDREDFLRKQRTFDNGSIPHQIHL | |
| | QELHAILRRQAEYYPFLKENAEKIEKILTFRIPYYVGPLARGNSRFAWMTRK | |
| | SNETITPWNFEEVVDKEASAEAFIERMTNFDKNLPEEKVLPKHSLLYETFTV | |
| | YNELTKVKYVTEGMGKPEFFDANQKQEIVDLLFKKYRKVTKKKLLDFLFKEF | |
| | EEFRIVDISGVEDAFNASLGTYHDLLKIIKDKEFLDNEENEDILEDIILTLT | |
| | LFEDREMIEERLSKYADLFDKKVLKKLERRRYTGWGRLSAKLINGIRDKQSG | |
| | KTILDYLIDDGYANRNFMQLIHDDSLSFKEEIKKAQVGGQGDSLHEHIANLA | |
| | GSPAIKKGILQTVKVVDELVKVMGRHKPENIVIEMARENQTTQKGQRNSRER | |
| | LKRLEEAIKELGSQILKEHPVENTQLQNDKLYLYYLQNGRDMYTGEELDIDR | |
| | LSDYDVDHIIPQSFIKDDSIDNKVLTSSAKARGKSDNVPSEEVVKKMKSFWK | |
| | KLLNSKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKKKKLKTVKELVGITIMERSAFEKNPVAFLEDKGYKNIQEDKII | |
| | KLPKYSLFELENGRKRLLASAGELQKGNELALPNKYVTLLYHASHYEKLKGK | |
| | PEDLPKHLEYVEQHRNEFKEILDQISEFAERYVLADKNIEKIKALYEENESF | |
| | SIEEIATSFINLLKFTALGAPAAFKFFGTTIDRKRYTSTKEILNATLIHQSI | |
| | TGLYETRIDLSKLGGD | |
| CasEnd-206 | MDKPYSIGLDIGTNSVGWAVVTDEYKVPSKKFKVLGNTDRKSIKKNLLGALL | 166 |
| | FDSGETAEATRLKRTARRRYTRRKNRILYLQEIFAEEMNKVDESFFHRLEES | |
| | FLVEEDKRGERHPIFGNIVEEVAYHEKYPTIYHLRKHLADSDEKADLRLVYL | |
| | ALAHIIKFRGHFLIEGDLDAENTDVQELFKELLEVYDRTFEESHLEEENVDA | |
| | ESILTEKISKSRRLEKLLALFPNEKKNGLFGEFLKLIVGLTPNFKSNFGLEE | |
| | DAKLQFSKDTYDEDLEELLGQIGDEYAELFVAAKKLYDAILLSGILTVKDNS | |
| | TKAPLSASMVKRYDEHHQDLTLLKKFIRKQLPEKYKEIFFDQSKNGYAGYID | |
| | GGASQEDFYKYLKKLLEKIDGSEYFLDKIEREDFLRKQRTFDNGSIPHQIHL | |
| | QELHAIIRRQGEYYPFLKENQEKIEKILTFRIPYYVGPLARGNSRFAWLSRK | |
| | SDETITPWNFDEIVDKEASAEAFIERMTNFDKNLPDEKVLPKHSLLYEKFTV | |
| | YNELTKVKYVTEGGKKPEFFDANMKQEIFDGVFKKYRKVTKKQLLDYLKKEF | |
| | DEFRIVDISGVEDRFNASLGTYHDLKKILDDKDFLDDEANEKILEDIILTLT | |
| | LFEDREMIKKRLEKYSDLLDKEQLKKLERRRYTGWGRLSAKLINGIRDKETG | |
| | KTILDYLIDDGNSNRNFMQLIHDDNLSFKEEIAKAQVIGDTESLHEVIANLA | |
| | GSPAIKKGILQSLKIVDELVKVMGRHEPENIVVEMARENQTTQKGQKNSRER | |
| | MKRLEESIKELGSDILKEHPVDNTKLQNDKLYLYYLQNGRDMYTGEELDIDK | |
| | LSDYDVDHIVPQSFLKDDSIDNRVLVSSAKARGKSDDVPSEEVVNKMKGFWK | |
| | KLLDAKLITQRKYDNLTKAERGGLTELDKAGFIKRQLVETRQITKHVAQILD | |
| | ERFNTEYDENGKLIRDVKIVTLKSKLVSQFRKEFELYKVREINNYHHAHDAY | |
| | LNAVVGKALIKKYPKLESEFVYGDYPVYDVKKLIKKSDKEIGKATAKMFFYS | |
| | NIMNFFKTDVKLADGTVVERPDIEVNDETGEIAWDKEKHIATVKKVLSMPQV | |
| | NIVKKTEVQTGGFSKESILPKRDSDKLIARKKDWDTKKYGGFDSPTVAYSVL | |
| | VIADIEKGKAKKLKTVKELVGITIMERSAFEKNPVAFLEDKGYQNIKKENII | |
| | KLPKYSLFELENGRKRLLASAGELQKGNELALPQHYVTLLYHAKHYEKLKGK | |
| | PEDIEYHLIYVEEHRDEFDELLDQISEFSKRYILADANLEKIKKLYEKNKEA | |
| | SIEELAKSFINLLTFTALGAPAAFKFFGKNIDRKRYTSTKEILNATLIHQSI | |
| | TGLYETRIDLSKLGED | |
| CasEnd-207 | MDKKYSIGLDIGTNSVGWAVITDEYKVPSKKFKVLGNTDRHSIKKNLIGALL | 167 |
| | FDSGETAEATRLKRTARRRYTRRKNRICYLQEIFSNEMAKVDPSFFKRLEES | |
| | FLVEEDKSGSRHPIFGNIVEEVAYHEKYPTIYHLRKKLVDSDEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVDTTE | |
| | TKAPLSASMIKRYDDHHQDLTLLKELVRKELPEKYKTIFFDQNANGYAGYID | |
| | GGATQEEFYAAIKPILESMSGTKELLEKLENRDLLRKQRTFDNGSIPHQIHL | |
| | GELRAILERQEKFYPFLKENREKIEKILSFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKKPVGKKKKLVKVKELLGITIMERSEFEKDPLGFLEKKGYTDVKMDEII | |
| | KLPKYSLFELGNGRKRMLASAGELQKGNELALPSEYVNFLYLASNYEKLKGT | |
| | PEEQKKKQKYVEENKSYLDEIIKQISEFSERVIKADANLQKVKAAYEKHKDK | |
| | PIQEQAENIIHLFTLTALGAPAAFKYFDETIDRKRYTSTKEVLDATLIHQSI | |
| | TGLYETRIDLSFLGGD | |
| CasEnd-208 | MDKKYSIGLDIGTNSVGWAVITDEYKVPSKKFKVLGNTDRHSIKKNLIGALL | 168 |
| | FDSGETAEATRLKRTARRRYTRRKNRICYLQEIFSNEMAKVDDSFFHRLEES | |
| | FLVEEDKRNERHPIFGNIVDEVAYHEKYPTIYHLRKHLADSTEKADLRLVYL | |
| | ALAHIIKFRGHFLIEGDLNSENSDVQKLFKQFVQTYNQTFEENPLNEETVDA | |
| | ESILTEKLSKSRRLENLIAQFPNEKKNGLFGNLIALSLGLTPNFKSNFDLSE | |
| | DAKLQFSKDTYDEDLENLLGQIGDEYADLFLAAKNLYDAILLSGILTVNDES | |
| | TKAPLSASMIKRYDEHHQDLTLLKAFVREQLPEKYKEIFFDKSKNGYAGYID | |
| | GGASQEEFYKYIKPILEKLDGSEYFLEKIEREDFLRKQRTFDNGSIPHQIHL | |
| | GELHAILRRQEEYYPFLKENQEKIEKILTFRIPYYVGPLARGNSRFAWATRK | |
| | SDETITPWNFEEVIDKEASAQAFIERMTNNDKNLPDEKVLPKHSLLYEKFTV | |
| | YNELTKVKYVTEGMGKPEFFSANQKEEIVDLLFKKERKVTKKKLLEFLFKEF | |
| | EEFRIVDISGVEDAFNASLGTYHDLLKIIKDKDELDNEENEDILEDIVLTLT | |
| | LFEDREMIEKRLEKYADLFDKKVLKKLKRRRYTGWGRLSRKLINGIRDKQSG | |
| | KTILDYLIDDGFANRNFMQLIHDDSLSFKEEIKKAQVIGDTDSLHEHIANLA | |
| | GSPAIKKGILQTVKVVDELVKVMGRHKPENIVIEMARENQTTQKGQRNSRER | |
| | LKRLEEGIKELGSDILKEHPVENTQLQNDKLYLYYLQNGKDMYTGEELDIDR | |
| | LSDYDVDHIIPQSFIKDDSIDNRVLTSSAKARGKSDNVPSEEVVKKMKSFWY | |
| | KLLKAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVAKIEKGKAKKLKTVKELVGITIMERSSFEKNPVAFLEAKGYQNIQKDKII | |
| | KLPKYSLFELENGRKRLLASAGELQKGNELALPQKYVTLLYHAKHYEKLKGK | |
| | PEDEEQHLEYVEQHRDEFDEILEQISEFSERYILADKNLEKIEELYEKNENF | |
| | SIEELAESFINLLTLTALGAPAAFKFLGTTIDRKRYTSTTEILNSTLIHQSI | |
| | TGLYETRIDLSKLGGD | |
| CasEnd-209 | MDKKYSIGLDIGTNSVGWAVITDEYKVPSKKFKVLGNTDRHSIKKNLIGALL | 169 |
| | FDSGETAEATRLKRTARRRYTRRKNRICYLQEIFSNEMAKVDDSFFHRLEES | |
| | FLVPEDKRNERHPIFGNIVDEVAYHEKYPTIYHLRKKLADSDEKADLRLIYL | |
| | ALAHMIKFRGHFLIEGDLNSDNSDVQKLFKQLVQTYNQLFEESPLNEEGVDA | |
| | KAILSARLSKSRRLENLIAQFPGEKKNGLFGNLIALSLGLTPNFKSNEDLAE | |
| | DAKLQLSKDTYDEDLENLLGQIGDQYADLFLAAKNLYDAILLSGILTVNDES | |
| | TKAPLSASMVKRYDEHHKDLKLLKKLVRQQLPEKYKEIFSDKSKNGYAGYID | |
| | GKTSQEEFYKYIKPILEKVDGSEEFLEKINREDFLRKQRTFDNGSIPHQIHL | |
| | QELHAILRRQEKYYPFLKENREKIEKILTFRIPYYVGPLARGNSRFAWMSRK | |
| | SDEKITPWNFDEVVDKEASAQAFIERMTNNDLYLPDEKVLPKHSLLYEKFTV | |
| | YNELTKVKYVTEGMRKPQFLSAEQKQEIVDLLFKKNRKVTVKKLKEDYFKKF | |
| | ECFDSVEISGVEDRFNASLGTYHDLLKIIKDKDFLDNEENEKILEDIVLTLT | |
| | LFEDREMIEERLKKYADLFDDKVLKQLKRRHYTGWGRLSAKLINGIRDKQSG | |
| | KTILDYLISDGFANRNFMQLIHDDSLTFKEEIEKAQVSGQGDSLHEHIANLA | |
| | GSPAIKKGILQTVKVVDELVKVMGRHKPENIVIEMARENQTTQKGQRNSRQR | |
| | LKRLEEGIKELGSDILKEYPVENTQLQNDRLYLYYLQNGKDMYTGEELDIDR | |
| | LSQYDIDHIIPQSFIKDDSIDNKVLVSSAKARGKSDNVPSEEVVKKMKNYWR | |
| | QLLDAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-210 | MDKSYSIGLDIGTNSVGWAVITDEYKVPSKKFKVLGNTGRKSIKKNLLGALL | 170 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLEDDKVMKQLKRRRYTGWGRLSRKLINGIRDKQSG | |
| | KTILDFLKSDGFANRNFMQLIHDDSLTFKEDIQKAQVSGQGESLHEQIANLA | |
| | GSPAIKKGILQTIKIVDEIVKVMGRYAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLTELDKAGFIKRQLVETRQITKHVAQILD | |
| | ERFNTEYDDNGKLIRDTKIVTLKSKLVSQLRKDFGLYKIREVNNYHHAHDAY | |
| | LNAVVGKALIKKYPKLESEFVYGDYPVYDVAKLVKKSDKEIGKATAKMFFYS | |
| | NLMNFFKSDVSLADGTLKKRPLIEVNEETGEIIWDKEKHIETIKKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-211 | MDKKYSIGLDIGTNSVGWAVITDEYKVPSKKFKVLGNTDRHSIKKNLIGALL | 171 |
| | FDSGETAEATRLKRTARRRYTRRKNRICYLQEIFSNEMAKVDDSFFHRLEES | |
| | FLVEEDKRGERHPIFGNIVEEVAYHEKYPTIYHLRKHLADSTEKADLRLIYL | |
| | ALAHIIKFRGHFLIEGDLNSENTDVQKLFIQFVQTYNQTFEENPLSEETVDA | |
| | KSILTAKLSKSRKLENLIAQFPNEKKNGLFGNLIALSLGLTPNFKSNFELSE | |
| | DAKLQFSKDTYDEDLENLLGQIGDEYADLFVAAKNLYDAILLSGILTVNDES | |
| | TKAPLSASMIKRYDEHHQDLTLLKKFVRQQLPEKYKEIFFDESKNGYAGYID | |
| | GGASQEEFYKYIKPILSKVDGAEYFLEKIEREDFLRKQRTFDNGSIPHQIHL | |
| | QELHAILRRQEEFYPFLKENQEKIEKILTFRIPYYVGPLARGNSRFAWMTRK | |
| | SDETITPWNFEEVVDKEASAEAFIERMTNFDKNLPDEKVLPKHSLLYEKFTV | |
| | YNELTKVKYVTEGMGKPEFFSANQKQEIFDELFKKNRKVTKKKLLEFLFKEF | |
| | ECFRIVEISGVEDAFNASLGTYHDLLKIIKDKDFLDNEENEDILEDIVLTLT | |
| | LFEDREMIEKRLSKYADLFDKKVLKKLKRRRYTGWGRLSKKLINGIRDKQSG | |
| | KTILDYLIDDGFANRNFMQLIHDDSLTFKEEIKKAQVIGQGDSLHEHIANLA | |
| | GSPAIKKGILQTVKVVDELVKVMGRHKPENIVIEMARENQTTQKGQRNSRER | |
| | LKRLEEAIKELGSDILKEHPVENTQLQNDKLYLYYLQNGKDMYTGEELDIDR | |
| | LSDYDVDHIIPQSFIKDDSIDNRVLTSSAKARGKSDNVPSEEVVKKMKSFWE | |
| | QLLKAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADVEKGKSKKLKTVKELVGITIMERSAFEKNPVAFLEDKGYQNIQKDLII | |
| | KLPKYSLFELENGRKRLLASAGELQKGNELALPNKYVTLLYHAKHYEKLKGK | |
| | PEDEEKHREYVEKHRDEFDEILDQISEFSKRYILADKNLEKILELYSKNENY | |
| | SIEELASSFINLLTFTALGAPAAFKFFGSTIDRKRYTSTKEILNATLIHQSI | |
| | TGLYETRIDLSKLGGD | |
| CasEnd-212 | MDKSYSIGLDIGTNSVGWAVITDEYKVPSKKFKVLGNTDRHSIKKNLLGALL | 172 |
| | FDSGETAEATRLKRTARRRYTRRKNRILYLQEIFAEEMSKVDDSFFHRLEES | |
| | FLVEEDKRGERHPIFGNIVDEVAYHENYPTIYHLRKKLADSTEKADLRLIYL | |
| | ALAHIIKFRGHFLIEGDLNSENTDVQKLFKQLVQTYDQLFEESHLSEETVDA | |
| | SDILTAKLSKSRRLENLIAQFPNEKKNGLFGNLIALSLGLTPNFKSNFKLSE | |
| | DAKLQFSKDTYDEDLENLLAQIGDQYADLFLAAKNLSDAILLSGILTVNTEI | |
| | TKAPLSASMIKRYDEHHQDLTLLKKLVRKQLPEKYKEIFFDDSKNGYAGYID | |
| | GGASQEEFYKYIKPILSKLDGSEYFLDKIEREDFLRKQRTFDNGSIPHQIHL | |
| | QELHAILRRQEKFYPFLKENQEKIEKILTFRIPYYVGPLARGNSRFAWMSRK | |
| | SDETITPWNFDEVVDKEASAQAFIERMTNNDKNLPNEKVLPKHSLLYEKFTV | |
| | YNELTKVKYITEGMRKPAFFSAEQKQEIVDLLFKKNRKVTVKKLKEYLFKKI | |
| | ECFDSVEISGVEDAFNASLGTYHDLLKIIKDKDFLDNEENEKILEDIVLTLT | |
| | LFEDREMIEQRLSKYADLFDKKVLKKLKRRRYTGWGRLSAKLINGIRDKQSG | |
| | KTILDYLIDDGFANRNFMQLIHDDSLTFKEEIEKAQVIGKGDSLHEVIANLA | |
| | GSPAIKKGILQSLKIVDELVKVMGRHNPENIVIEMARENQTTQKGQKNSRER | |
| | LKRLEESIKELGSKILKEHPVDNTQLQNDKLYLYYLQNGKDMYTGEELDIDR | |
| | LSDYDVDHIIPQSFIKDDSIDNRVLTSSEKARGKSDNVPSIEVVKKMKSFWR | |
| | KLLNAKLISQRKFDNLTKAERGGLSELDKAGFIKRQLVETRQITKHVAQILD | |
| | SRFNTKYDENDKLIRKVKIITLKSKLVSDFRKEFGFYKVREINDYHHAHDAY | |
| | LNAVVGTALIKKYPKLESEFVYGDYPVYDSYKMIAKSEQEIGKATAKYFFYS | |
| | NIMNFFKSEITLANGEIRKRPLIETNEETGEIVWDKEKDFATVKKVLSMPQV | |
| | NIVKKTEVQTGGFSKESILPKRNSDKLIARKKDWDTKKYGGFDSPTVAYSVL | |
| | VVADVEKGKSKKLKTVKELVGITIMERSSFEKNPIAFLEAKGYKNIQKELII | |
| | KLPKYSLFELENGRKRLLASAGELQKGNELALPGKYVTLLYHAKHYEKLKGS | |
| | PEDNEEHREYVEQHREEFKEIFDQISEFSERYILADKNLEKILELYAENEDS | |
| | SIEELASSFINLLTFTALGAPAAFKFFDQDIDRKRYTSTTEILNATLIHQSI | |
| | TGLYETRIDLSQLGGD | |
| CasEnd-213 | MDKKYSIGLDIGTNSVGWAVITDEYKVPSKKFKVLGNTDRHSIKKNLLGALL | 173 |
| | FDSGETAEATRLKRTARRRYTRRKNRIRYLQEIFAEEMAKVDDSFFHRLEES | |
| | FLVEEDKRHERHPIFGNIVDEVAYHEKYPTIYHLRKKLADSTDKADLRLIYL | |
| | ALAHMIKFRGHFLIEGDLNPDNSDVQKLFYQLVQTYNQLFEESPIDISGVDA | |
| | KAILSAKLSKSRRLENLIAQFPGEKKNGLFGNLIALSLGLTPNFKSNEDLAE | |
| | DAKLQLSKDTYDEDLDNLLAQIGDQYADLFLAAKNLSDAILLSDILTVNSEV | |
| | TKAPLSASMIKRYDEHHQDLTLLKELVRQQLPEKYKEIFFDQTKNGYAGYID | |
| | GGASQEEFYKYIKPILEKMDGTEDLLSKLNREDFLRKQRTFDNGSIPHQIHL | |
| | NELHAILRRQEDFYPFLKENQEKIEKILTFRIPYYVGPLARGNSRFAWLTRK | |
| | SEETITPWNFEEVVDKGASAQSFIERMTNFDKNLPHEKVLPKHSLLYEYFTV | |
| | YNELTKVKYVTEGMRKPEFLSAGQKEAIVDLLFKTNRKVTVKQLKEDYFKKI | |
| | ECFDTVEISGVEDKFNASLGTYHDLLKIIKDKDFLDNEENEDILEDIVLTLT | |
| | LFEDREMIEERLEKYAHLFDKKVLKQLKRRRYTGWGRLSRKLINGIRDKQSG | |
| | KTILDFLKSDGFANRNFMQLIHDDSLTFKEAIKKAQVIGQGDSLHEQIANLA | |
| | GSPAIKKGILQSVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINDYHHAHDAY | |
| | LNAVVGTALIKKYPKLESEFVYGDYKVYDVRKMIAKSEQEIGKATAKYFFYS | |
| | NIMNFFKSEVTLANGEIRKRPLIEMNEETGEIVWDKGKDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELVGITIMERSSFEKNPVDFLEAKGYKNIQKDLII | |
| | KLPKYSLFELENGRKRLLASAGELQKGNELALPQKYVTFLYHAKNYEKIKGS | |
| | EEDREKKLEYVEQHRHEFDEILSQIEEFSKRYILADKNLSKIKELYNNEADK | |
| | SISELAENFIHLFTFTSLGAPAAFKFFDTTIDRKRYTSTTEVLDATLIHQSI | |
| | TGLYETRIDLSQLGGD | |
| CasEnd-214 | MKKPYSIGLDIGTNSVGWAVVTDDYKVPAKKMKVLGNTDKSHIKKNLLGALL | 174 |
| | FDSGNTAEDRRLKRTARRRYTRRRNRILYLQEIFAEEMNKVDDSFFHRLDDS | |
| | FLVTEDKRGERHPIFGNLAEEVKYHENFPTIYHLRKHLADSPEKADLRLVYL | |
| | ALAHIIKYRGHFLIEGKLDTENKDVQELFQEFLAVYDNTFEDSSLQDQNVQI | |
| | EEILTDKISKSAKKDRVLKLFPNEKSNGFFAEFLKLIVGNQADFKKNFELEE | |
| | KAPLQFSKDSYEEDLEVLLGQIGDNYADLFVAAKKLYDSILLSGILTVNDVS | |
| | TKAPLSASMIQRYEEHQEDLAQLKCFIRKKLSEKYNEVFSDKSKDGYAGYID | |
| | GKTNQEAFYKYLKKLLNKVEGSGYFLEKIEREDFLRKQRTFDNGSIPHQIHL | |
| | QEMRAIIRRQAEYYPFLAENQDKIEQILTFRIPYYVGPLARGKSDFAWLSRK | |
| | SDEKITPWNFDEIVDKESSAEAFINRMTNYDLYLPEQKVLPKHSLLYEKFTV | |
| | YNELTKVRYKTEQMGKTHFFDANMKQEIFDGVFKKYRKVTKKKLMDFLHKEF | |
| | DEFRIVDLTGLDKQFNASYGTYHDLLKILQDKDFLDDPKNEKILEDIVLTLT | |
| | LFEDREMIRKRLSKYSDLLTKEQVKKLERRHYTGWGRLSAKLINGIRNKETR | |
| | KTILDYLIDDGNSNRNFMQLINDDALSFKEEIAKAQVIGETDNLNQVVQDLA | |
| | GSPAIKKGILQSLKIVDELVKIMGRYNPENIVVEMARENQFTNQGRRNSQQR | |
| | LKGLTDSIKELGSQILKEHPVDNSQLQNDRLFLYYLQNGRDMYTGEELDIDK | |
| | LSQYDIDHIIPQAFIKDDSIDNRVLVSSAKARGKSDDVPSKEVVKKMKSFWQ | |
| | KLLDAKLISQRKFDNLTKAERGGLTDDDKAGFIKRQLVETRQITKHVARILD | |
| | ERFNTETDENNKKIRKVKIVILKSNLVSNFRKEFELYKVREINDYHHAHDAY | |
| | LNAVVAKALLGKYPKLEPEFVYGEYPKYNSYRYVDETNKERKKATAKMFFYS | |
| | NIMNFFKSDVKLADGSVVERPMIEVNNETGEIVWDKTKHISTVKKVLSYPQV | |
| | NIVKKVEEQTGGFSKENILPKGNSDKLIPRKTKWDTKKYGGFDSPIVAYSVL | |
| | VIADIEKGKAKKLKTVKELIGITIMEKMTFEKDPVAFLERKGYQNIQEENII | |
| | KLPKYSLFELENGRRRLLASARELQKGNEIVLPNHLVTLLYHAKNIDKVSEK | |
| | AKDVPKHLQYVEKHRSEFKELLDEIMNFSKKYTLAEANLEKIIELYADNNQA | |
| | SIEEIASSFINLLTFTALGAPAAFKFLDKNIDRKRYTSTTEILNATLIHQSI | |
| | TGLYETRIDLSKLGED | |
| CasEnd-215 | MDKKYSIGLDIGTNSVGWAVITDEYKVPSKKFKVLGNTDRHSIKKNLIGALL | 175 |
| | FDSGETAEATRLKRTARRRYTRRKNRICYLQEIFSNEMAKVDPSFFQRLEES | |
| | FLVEEDKRGERHPIFGNIVEEVAYHEKYPTIYHLRKELADSDEKADLRLVYL | |
| | ALAHMIKFRGHFLIEGDLDAENSDVQKLFLTLIETYDQTFEESPLEEEEIDA | |
| | EAILTEKLSKSRRLENLIAKFPGEKKNSLFGNLIGLALGLTPNFKSNFDLSE | |
| | DAKLQFSKDTYDEDLEELLGQIGDEYADLFLAAKNLYDAILLSGILTVDDNS | |
| | TKAPLSASMVKRYDEHHQDLTLLKEFVRKQLPEKYKEIFFDQTKNGYAGYID | |
| | GGASQEEFYKYLKKLLEKIDGSEYFLDKIEREDFLRKQRTFDNGSIPHQIHL | |
| | QELHAILRRQEEYYPFLKENQEKIEKILTFRIPYYVGPLARGNSRFAWMSRK | |
| | SDETITPWNFDDVVDKEKSAEKFIERMTNFDKNLPEEKVLPKHSLLYEKFTV | |
| | YNELTKVKYVTEGGKKPKFFDAELKQEIFDDLFKKERKVTKKQLLEYLYKEF | |
| | DEFRIVEISGVEDRFNASLGTYHDLLKIIKDKSFLDNSENEEILEDIILTLT | |
| | LFEDREMIKKRLEKYSDLFDKKQLKKLSRRRYTGWGRLSAKLINGIRDKQTG | |
| | KTILDYLIDDGFTNRNFMQLIHDDNLTFKEEISKAQVIKDTDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-216 | MDKKYSIGLDIGTNSVGWAVITDEYKVPSKKFKVLGNTDRHSIKKNLIGALL | 176 |
| | FDSGETAEATRLKRTARRRYTRRKNRICYLQEIFSNEMAKVDDSFFHRLEES | |
| | FLVEEDKRHERHPIFGNIVDEVAYHEKYPTIYHLRKELADSTEKADLRLVYL | |
| | ALAHIIKFRGHFLIEGDLNPENTDVQKLFIQFVQTYNRTFEESPLSEETVDA | |
| | KSILTEKLSKSRRLENLIAQFPNEKKNGLFGNLIALSLGLTPNFKSNFELSE | |
| | DAKLQFSKDTYDEDLENLLGQIGDEYADLFLAAKNLYDAILLSGILTVNDES | |
| | TKAPLSASMIKRYDEHHQDLTLLKAFVREQLPEKYKEIFFDKSKNGYAGYID | |
| | GGASQEEFYKYIKKILEKIDGSEYFLAKIEREDFLRKQRTFDNGSIPHQIHL | |
| | GELHAILRRQGEYYPFLKENQEKIEKILTFRIPYYVGPLARGNSRFAWMSRK | |
| | SDETITPWNFEEVVDKEASAEAFIERMTNFDKNLPEEKVLPKHSLLYEKFTV | |
| | YNELTKVKYVTEGMGKAEFFDANMKQEIFDGLFKKNRKVTKKKLLDFLDKEF | |
| | DEFRIVDISGVEKAFNASLGTYHDLLKIIKDKDFLDNEENEDILEDIILTLT | |
| | LFEDREMIEERLSKYADLFDKKVLKKLERRRYTGWGRLSAKLINGIRDKQSG | |
| | KTILDYLIDDGNANRNFMQLIHDDSLTFKEEIKKAQVIGESDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKDPVAFLEDKGYQNVQEDKII | |
| | KLPKYSLFELENGRKRLLASAGELQKGNELALPNKYVTLLYHAKHYEKLKEK | |
| | PEDEEKHLEYVDKHRDEFDEILDQISEFSERYILADKNLEKIKELYAKFESY | |
| | SIEELASSFINLLTFTALGAPAAFKFLGSTIDRKRYTSTKEILNATLIHQSI | |
| | TGLYETRIDLSKLGGD | |
| CasEnd-217 | MKKKYSIGLDIGTNSVGWAVITDEYKVPSKKFKVLGNTDRQSIKKNLLGALL | 177 |
| | FDSGETAEATRLKRTARRRYTRRKNRIRYLQEIFAEEMSKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENTDVQKLFIQLVQTYNQLFEENPINEEGIDA | |
| | KAILTAKLSKSRRLENLIAQLPGEKRNGLFGNLIALSLGLTPNFKSNEDLAE | |
| | DAKLQLSKDTYDEDLDNLLAQIGDQYADLFLAAKNLSDAILLSDILTVNDEI | |
| | TKAPLSASMIKRYDEHHQDLTLLKALVRQQLPEKYKEIFFDQSKNGYAGYID | |
| | GGASQEEFYKYIKPILEKMDGTEELLAKLNREDFLRKQRTFDNGSIPHQIHL | |
| | GELHAILRRQEDFYPFLKENQEKIEKILTFRIPYYVGPLARGNSRFAWMSRK | |
| | SEETITPWNFEEVVDKEASAQAFIERMTNFDKNLPSEKVLPKHSLLYEKFTV | |
| | YNELTKVKYVTEGMRKPHFLSSEQKEEIVDLLFKKNRKVTVKQLKEDYFSKI | |
| | ECFDSVEISGVEDKFNASLGTYHDLLKIIKDKDFLDNEENEDILEDIVLTLT | |
| | LFEDKEMIEERLAKYAHLFDKKVMKKLKRRRYTGWGRLSRKLINGIKDKQSG | |
| | KTILDFLKSDGFANRNFMQLIHDDSLTFKEEIEKAQVKGQGESLHEQIANLA | |
| | GSPAIKKGILQSVKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEFDENNKLIREVKIITLKSKLVSDFRKDFGFYKVREINDYHHAHDAY | |
| | LNAVVGTALIKKYPKLESEFVYGDYKVYDVRKLIAKSEQEIGKATAKYFFYS | |
| | NIMNFFKSEVTLANGEIRKRPLIETNEETGEIVWDKEKDFATVKKVLSMPQV | |
| | NIVKKTEVQTGGFSKESILPKRNSDKLIARKKDWDTKKYGGFDSPTVAYSVL | |
| | VVAKVEKGKAKKLKSVKELVGITIMERSSFEKDPIAFLEDKGYKNVQKDLII | |
| | KLPKYSLFELENGRKRLLASAGELQKGNELALPNKYVNFLYHASHYEKLKGK | |
| | SEDNEKKRLYVEEHRHYFDEIFEQIIEFSERYILADANLEKIKSLFKENEDK | |
| | SISELAENFIHLFTLTALGAPAAFKFFDKDIDRKRYTSTTEILNATLIHQSI | |
| | TGLYETRIDLSQLGGD | |
| CasEnd-218 | MKKPYSIGLDIGTNSVGWAVVTDDYKVPSKKMKVLGNTDRSSIKKNLLGALL | 178 |
| | FDSGETAEATRLKRTARRRYTRRKNRILYLQEIFAEEMNKVDESFFHRLEDS | |
| | FLVEEDKRGERHPIFGTIVEEVKYHEEFPTIYHLRKHLADSPEKADLRLVYL | |
| | ALAHIIKFRGHFLIEGDLDTENTDVQSLFKEFLEVYDETFENSHLSEETVDV | |
| | EEILTDKISKSRKKERLLKLFPTEKSNGQFAEFLKLIVGNQANFKKVFELSE | |
| | KAKLQFSKDTYEEDLEILLGKIGDEYADVFVSAKNLYDSILLSGILTVTDLS | |
| | TKAPLSASMVKRYEEHHEDLTKLKKFIRENLPEKYKEVFFDESKNGYAGYID | |
| | GGTKQEDFYKYLKKLLSKIAGSEYFLEKIEREDFLRKQRTFDNGSIPHQIHL | |
| | QEMKAIIRRQAEYYPFLKENQDKIEQILTFRIPYYVGPLARGNSRFAWASRK | |
| | SDETITPWNFDDIIDKEKSAEAFINRMTNYDLYLPEEKVLPKHSLLYEKFTV | |
| | YNELTKVKYITEQGGKTEFFDANMKQEIFDGVFKKERKVTKKKLLNFLDKEF | |
| | DEFRIVDLSGVEKAFNASLGTYHDLKKILGDKEFLDDPENEGMLEDIVLTLT | |
| | LFEDREMIKKRLEKYSDIFTKEQLKKLERRHYTGWGRLSAKLINGIRDKETN | |
| | KTILDYLIDDGYSNRNFMQLIHDDALSFKEEIAKAQVIGETDSLHEVVAELA | |
| | GSPAIKKGILQSLKIVDELVKVMGRYNPENIVVEMARENQTTNKGQRNSRER | |
| | LKGLTDAIKELGSDILKEHPVDNQQLQNDRLYLYYLQNGKDMYTGETLDIDN | |
| | LSQYDVDHIIPQSFIKDDSIDNRVLVSSAKARGKSDDVPSIEVVHKMKSEWN | |
| | KLLNAKLISQRKYDNLTKAERGGLTDDDKAGFIKRQLVETRQITKHVAQILD | |
| | ERFNTERDENNKLIRKVKIVTLKSKLVSNFRKDFELYKVREINDYHHAHDAY | |
| | LNAVVGKALITKYPQLEPEFVYGDYPKFNSYKLERKKDSERGKATAKMFFYS | |
| | NLMNFFKSDVKLADGTVVERPIIEVNDENGEIAWKKTKHVSNVKKVLSMPQV | |
| | NIVKKTEVQTGGFSKESILPKRNSDKLIARKKKWDTKKYGGFDSPIVAYSVL | |
| | VIADIEKGKAKKLKTVKELVGITIMEKSRFEKDPVAFLENKGYQNIQEENII | |
| | KLPKYSLFSLENGRKRLLASAGELQKGNELALPNHLVTLLYHAKNIEKDDEK | |
| | KKDIPKHLEYVKKHRSEFKELFDQVSEFSKRYILADKNLEKIEELYTQNEEA | |
| | DVKELASSFINLLTFTAIGAPADFKFFGKDIDRKRYTSTTECLNATLIHQSI | |
| | TGLYETRIDLSKLGED | |
| CasEnd-219 | MDKKYSIGLDIGTNSVGWAVITDEYKVPSKKFKVLGNTDRHSIKKNLIGALL | 179 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNPENSDVDKLFIQLVQTYNQLFEENPINESGVDA | |
| | KAILSAKLSKSRRLENLIAQFPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKYIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SDETITPWNFEEVVDKGASAQAFIERMTNFDKNLPNEKVLPKHSLLYEYFTV | |
| | YNELTKVKYVTEGMRKPEFLSGEQKEEIVDLLFKKNRKVTVKQLKDDYFKEI | |
| | ECFDSVEISGVEDAFNASLGTYHDLLKIIKDKDFLDNEENEDILEDIVLTLT | |
| | LFEDREMIEERLKKYANLFDDKVLKQLKRRRYTGWGRLSRKLINGIRDKQSG | |
| | KTILDFLKSDGFANRNFMQLIHDDSLTFKEEIKKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEAKGYKNVQKDLII | |
| | KLPKYSLFELENGRKRMLASAGELQKGNELALPSKYVTLLYHASNYEKLKGK | |
| | PEDEEKKLEYVEQHRHYFDEIFDQISEFSERYILADKNLEKILSLYNKFEDK | |
| | SIREQAENFINLFTLTALGAPAAFKFFGTTIDRKRYTSTKEILNSTLIHQSI | |
| | TGLYETRIDLSQLGGD | |
| CasEnd-220 | MKKPYSIGLDIGTNSVGWAVVTDDYKVPSKKMKVLGNTDRSSIKKNLLGALL | 180 |
| | FDSGETAEATRLKRTARRRYTRRKNRILYLQEIFAEEMNKVDDSFFHRLEDS | |
| | FLVEEDKRGERHPIFGNIVDEVAYHEKFPTIYHLRKHLADSTEKADLRLVYL | |
| | ALAHIIKFRGHFLIEGDLDSENTDVQKLFKQFVEAYDRTFEESHLSEETVDA | |
| | EAILTEKISKSRKLENLLKQFPNEKKNGFFGNLIALSLGLQPNFKKNFGLSE | |
| | DAKLQFSKDTYDEDLENLLGQIGDEYADLFVAAKNLYDAILLSGILTVNDSS | |
| | TKAPLSASMIKRYDEHHEDLTLLKKFIRKQLPEKYKEIFFDESKNGYAGYID | |
| | GGTSQEEFYKYIKPILSKIDGSEYFLDKIEREDFLRKQRTFDNGSIPHQIHL | |
| | QELKAILRRQGEYYPFLKENQEKIEKILTFRIPYYVGPLARGNSRFAWMERK | |
| | SDETITPWNFDEVVDKEKSAEAFIERMTNNDLYLPEEKVLPKHSLLYEKFTV | |
| | YNELTKVKYVTEGMGKAQFFDANMKQEIFDGLFKKERKVTKKKLLDFLDKEF | |
| | DEFRIVDISGVEDRFNASLGTYHDLLKIIKDKDFLDNEENEEILEDIILTLT | |
| | LFEDREMIKKRLEKYADLFDKKVLKKLERRHYTGWGRLSAKLINGIRDKQTG | |
| | KTILDYLIDDGRANRNFMQLIHDDSLSFKEEIKKAQVIGQEDSLHEVVANLA | |
| | GSPAIKKGILQSLKIVDELVKVMGRYEPENIVVEMARENQTTAKGQRNSRER | |
| | LKRLEEAIKNLGSNILKEHPVENQQLQNDRLYLYYLQNGKDMYTGEELDIDK | |
| | LSQYDVDHIIPQSFIKDDSIDNRVLVSSAKARGKSDDVPSIEVVKKMKSFWS | |
| | KLLSAKLISQRKFDNLTKAERGGLTEEDKAGFIKRQLVETRQITKHVAQILD | |
| | ERFNTEFDENNKLIRKVKIITLKSKLVSDFRKEFEFYKVREINDYHHAHDAY | |
| | LNAVVGKALIKKYPKLEPEFVYGDYPKYNSFKMIAKSDKERGKATAKMFFYS | |
| | NIMNFFKTDVKLADGTIVERPVIEVNDETGEIVWDKEKHIATVKKVLSMPQV | |
| | NIVKKTEVQTGGFSKESILPKRNSDKLIARKKKWDTKKYGGFDSPTVAYSVL | |
| | VVADIEKGKSKKLKTVKELVGITIMERSRFEKNPVAFLEAKGYQNIQEEKII | |
| | KLPKYSLFELENGRKRLLASAGELQKGNELALPNKLVTLLYHAKRIEKLDEK | |
| | PEDLPKHLEYVEKHKSEFDELLNQVSEFSERYILADKNLEKIEELYKQNNDS | |
| | SIEELASSFINLLTFTALGAPADFKFFGTTIDRKRYTSTTEILNATLIHQSI | |
| | TGLYETRIDLSKLGGD | |
| CasEnd-221 | MDKKYSIGLDIGTNSVGWAVITDEYKVPSKKFKVLGNTDRHSIKKNLIGALL | 181 |
| | FDSGETAEATRLKRTARRRYTRRKNRICYLQEIFSNEMAKVDPSFFQRLEES | |
| | FLVEEDKSGSRHPIFGNIVEEVAYHEKYPTIYHLRKKLVDSKEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVDTSE | |
| | TRAPLSASMIKRYDDHHQDLTKLKELVRKELPEKYKTIFFDQNANGYAGYID | |
| | GGATQEEFYKAIKPILESMSGTKELLDKLEKKDLLRKQRTFDNGSIPHQIHL | |
| | GELRAILERQEKFYPFLKENRERIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLEDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKKEVGEEKELKEVKELLGITIMERSEFEKNPLAFLEKKGYKDVKMDKII | |
| | KLPKYSLFELGNGRKRMLASAGELQKGNELALPSEYVNFLYLASDYEKLKGK | |
| | EEEKKKKQEYVEKNKHYLDEIINQISEFSKRVIKADANLEKVLKAYEKHKDK | |
| | PIKEQAENIIHLFTLTRLGAPAAFKYFDEVIDRKRYTSTKEVLDATLIHQSI | |
| | TGLYETRIDLSFLGGD | |
| CasEnd-222 | MDKKYSIGLDIGTNSVGWAVITDEYKVPSKKFKVLGNTDRHSIKKNLIGALL | 182 |
| | FDSGETAEATRLKRTARRRYTRRKNRICYLQEIFSNEMAKVDDSFFHRLEES | |
| | FLVEEDKRNERHPIFGNIVDEVAYHEKYPTIYHLRKHLADSTEKADLRLIYL | |
| | ALAHIIKFRGHFLIEGDLNAENTDVQKLFEQFVQTYDNTFEESHLEEITVDA | |
| | EAILTDKLSKSRRLENLIKQFPNEKKNGLFGNLIALSLGLQPNFKSNFKLSE | |
| | DAKLQFSKDTYDEDLENLLGQIGDEYADLFVAAKNLYDAILLSGILTVNDVS | |
| | TKAPLSASMIKRYDEHHQDLTLLKKFVRKQLPEKYKEIFFDESKNGYAGYID | |
| | GGASQEEFYKYIKPLLEKMDGSEYFLDKIEREDFLRKQRTFDNGSIPHQIHL | |
| | QELHAILRRQEDYYPFLKENQEKIEKILTFRIPYYVGPLARGNSRFAWLSRK | |
| | SDETITPWNFEEVVDKEASAEAFIERMTNFDKNLPEEKVLPKHSLLYEKFTV | |
| | YNELTKVKYVTEGMGKPEFFDAEMKQEIFDGLFKKNRKVTKKKLLDFLFKEF | |
| | DEFRIVDISGVEKAFNASLGTYHDLLKIIKDKDFLDNEENEDILEDIILTLT | |
| | LFEDREMIEERLQKYADLFDKKQLKKLKRRRYTGWGRLSKKLINGIRDKQSG | |
| | KTILDYLIDDGFANRNFMQLIHDDSLSFKEEIKKAQVIGQGDSLHEHIANLA | |
| | GSPAIKKGILQTVKVVDELVKVMGRHKPENIVIEMARENQTTQKGQRNSRER | |
| | LKRLEESIKELGSDILKEHPVENTQLQNDKLYLYYLQNGKDMYTGEELDIDR | |
| | LSDYDVDHIIPQSFIKDDSIDNRVLTSSAKARGKSDDVPSEEVVKKMKSFWR | |
| | QLLKAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVAKIEKGKAKKLKTVKELVGITIMERSSFEKNPVAFLEKKGYKNIQEELII | |
| | KLPKYSLFELENGRKRLLASAGELQKGNELALPQKYVTLLYHAKNYEKLKEE | |
| | PEDKEKHLEYVEEHRSEFKEILDQISEFSKRYILADKNLEKIEELYEKNENA | |
| | SIEELASSFINLLTFTALGAPAAFKFFGTTIDRKRYTSTKEILNATLIHQSI | |
| | TGLYETRIDLSKLGGD | |
| CasEnd-223 | MDKKYSIGLDIGTNSVGWAVITDEYKVPSKKFKVLGNTDRHSIKKNLIGALL | 183 |
| | FDSGETAEATRLKRTARRRYTRRKNRIRYLQEIFSEEMSKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVQKLFIQLVQTYNQLFEENPINESGVDA | |
| | KAILSAKLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILTVNDEI | |
| | TKAPLSASMIKRYDEHHQDLTLLKALVRQQLPEKYKEIFFDQSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQAFIERMTNFDKNLPNEKVLPKHSLLYEYFTV | |
| | YNELTKVKYVTEGMRKPEFLSGEQKEEIVDLLFKKNRKVTVKQLKEDYFKKI | |
| | ECFDSVEISGVEDRFNASLGTYHDLLKIIKDKDELDNEENEDILEDIVLTLT | |
| | LFEDREMIENRLKKYAHLFDKKVLKQLKRRRYTGWGRLSRKLINGIRDKQSG | |
| | KTILDFLKSDGFANRNFMQLIHDDSLTFKEAIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEDKGYKEIQKDLII | |
| | KLPKYSLFELENGRKRLLASAGELQKGNELALPSKYVTFLYLASHYEKLKGK | |
| | PEDNEQKLEYVEQHKHYFDEIFQQISEFSERYILADKNLEKILELYNEHRDS | |
| | SIVELAENFIHLFTFTALGAPAAFKFFDTTIDRKRYTSTTEILNATLIHQSI | |
| | TGLYETRIDLSQLGGD | |
| CasEnd-224 | MKKKYSIGLDIGTNSVGWAVITDEYKVPSKKFKVLGNTDRQSIKKNLLGALL | 184 |
| | FDSGETAEATRLKRTARRRYTRRKNRIRYLQEIFAEEMSKVDDSFFHRLEES | |
| | FLVEEDKLYERHPIFGNIVDEVAYHEKYPTIYHLRKKLADSTEKADLRLIYL | |
| | ALAHIIKFRGHFLIEGDLNSDNTDVDKLFIQLVQTYNQLFEENPINEETVDA | |
| | KAILSAKLSKSRRLENLIALFPGEKRNGLFGNLIALSLGLTPNFKSNFDLAE | |
| | DAKLQLSKDTYDEDLDNLLAQIGDQYADLFLAAKNLSDAILLSGILTVKDGI | |
| | TKAPLSASMIKRYDEHHQDLTLLKKLVREQLPEKYKEIFFDESKNGYAGYID | |
| | GGASQEEFYKFIKPILEKMDGTEELLAKIDREDFLRKQRTFDNGSIPHQIHL | |
| | GELKAILRRQEKFYPFLKENREKIEKILTFRIPYYVGPLARGNSRFAWMIRK | |
| | SEETITPWNFEEVVDKEASAQAFIERMTNFDKNLPDEKVLPKHSLLYEYFTV | |
| | YNELTKVKYVTEGMKKPEFLSSEQKEAIVDLLFKKNRKVTVKQLKEFYFSKI | |
| | ECFDSVEISGVEDRFNASLGTYHDLLKIIKDKEFLDNEENEDILEDIVLTLT | |
| | LFEDKEMIEKRLKKYADLFDKKVLKQLKRRRYTGWGRLSRKLINGIRDKQSG | |
| | KTILDFLKSDGFANRNFMQLIHDDSLTFKEEIKKAQVSGQGDSLHEVIANLA | |
| | GSPAIKKGILQTVKIVDEIVKVMGRHAPENIVIEMARENQTTQKGQKNSRER | |
| | MKRLEEGIKELGSQILKEHPVENTQLQNDKLYLYYLQNGKDMYTGEELDIDR | |
| | LSDYDVDHIIPQSFIKDDSIDNKVLTSSEKARGKSDNVPSEEVVKKMKNYWR | |
| | QLLNAKLISQRKFDNLTKAERGGLSELDKAGFIKRQLVETRQITKHVAQILD | |
| | SRFNTKRDENDKLIRDVKIITLKSKLVSDFRKDFGFYKVREINDYHHAHDAY | |
| | LNAVVGTALIKKYPKLESEFVYGDYKVYDSRKMIAKSEQEIGKATAKRFFYS | |
| | NIMNFFKSEIKLADGEIIKRPLIETNEETGEIVWDKGKDFATVRKVLSMPQV | |
| | NIVKKTEVQTGGFSKESILPKRDSDKLIARKKDWDTKKYGGFDSPTVAYSVL | |
| | VVAKVEKGKSKKLKTVKELVGITIMERSSFEKDPIAFLEAKGYKDIQKDLII | |
| | KLPKYSLFELENGRKRLLASAGELQKGNELALPSKYVTFLYHASNYEKLKGK | |
| | PEDEEKKREYVEQHNHEFDEILDQISEFSKRYILADKNLEKILSLYNKFRDK | |
| | SIREQAENFINLFTLTALGAPAAFKFFDKTIDRKRYTSTKEILNATLIHQSI | |
| | TGLYETRIDLSKLGGD | |
| CasEnd-225 | MDKKYSIGLDIGTNSVGWAVITDEYKVPSKKFKVLGNTDRHSIKKNLIGALL | 185 |
| | FDSGETAEATRLKRTARRRYTRRKNRICYLQEIFSNEMAKVDPSFFERLEES | |
| | FLVEEDKKYSRHPIFGNIVEEVAYHEKYPTIYHLRKKLVDSEEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVDTKE | |
| | TKAPLSASMIKRYDDHHQDLTLLKELVRKELPEKYKEIFFDQNKAGYAGYID | |
| | GGATQEEFYKYIKPILESMSGTKELLEKLENRDLLRKQRTFDNGSIPHQIHL | |
| | GELRAILERQEKFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLEDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKKEVGKEKKLVEVKDLLGITIMERSKFEKDPLKFLEEKGYKDVKMDEII | |
| | KLPKYSLFELGNGRKRMLASAGELQKGNELALPSEYVNFLYLASNYEKLKGK | |
| | EEEKKKKQEYVEKNKSYLDDIINQISEFSKRVIGADANLEKVLAAYKKHKNK | |
| | PISEQAENIIHLFTLTRLGAPAAFKYFDETIDRKRYTSTKEVLDATLIHQSI | |
| | TGLYETRIDLSFLGGD | |
| CasEnd-226 | MDKKYSIGLDIGTNSVGWAVITDEYKVPSKKFKVLGNTDRHSIKKNLIGALL | 186 |
| | FDSGETAEATRLKRTARRRYTRRKNRICYLQEIFSNEMAKVDDSFFHRLEES | |
| | FLVEEDKQGDRHPIFGNIVEEVAYHEKYPTIYHLRKELADSTEKADLRLVYL | |
| | ALAHIIKFRGHFLIEGDLNPENTDVQKLFKDFVEIYNQTFEESPLNEEKVDA | |
| | KSILTEKLSKSRRLENLIAQFPNEKKNGLFGNLIALILGLQPNFKSNFQLAE | |
| | DAKLQFSKDTYDEDLENLLGQIGDEYADLFVAAKNLYDAILLSGILTVSDAT | |
| | TKAPLSASMIKRYDEHHQDLTLLKTFVRENLPEKYKEIFFDESKNGYAGYID | |
| | GGASQEEFYKYIKKLLEKIDGSEYFLEKIDREDFLRKQRTFDNGSIPHQIHL | |
| | QELHAILRRQEEFYPFLKENREKIEKILTFRIPYYVGPLARGNSRFAWMTRK | |
| | SDETITPWNFDEIVDKEKSAEAFIERMTNNDKNLPDEKVLPKHSLLYEKFTV | |
| | YNELTKVKYVTEGMGKPKFFDANQKQEIFDGLFKKYRKVTKKKLLDFLDKEF | |
| | DEFRIVDISGVEKAFNASLGTYHDLKKIIKDKAFLDNEENEKILEDIILTLT | |
| | LFEDREMIRQRLEKYADLFDKKQLKKLERRRYTGWGRLSAKLINGIRDKQTG | |
| | KTILDYLIDDGRANRNFMQLIHDDSLSFKEEIAKAQVAGEGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKSKKLKTVKELIGITIMERSKFEKDPVAFLEQKGYQNIKEDKII | |
| | KLPKYSLFELENGRKRLLASAGELQKGNELALPNKYVTLLYHAKHYEKLKEQ | |
| | QEDIEGHREYVEKHRDEFDELLDQINEFSERYILADKNLSKIEELYAQNLEY | |
| | SIEELANSFINLLTFTALGAPAAFKFFGNTIDRKRYTSTKEILNATLIHQSI | |
| | TGLYETRIDLSKLGGD | |
| CasEnd-227 | MDKKYSIGLDIGTNSVGWAVITDEYKVPSKKFKVLGNTDRHSIKKNLIGALL | 187 |
| | FDSGETAEATRLKRTARRRYTRRKNRICYLQEIFSNEMAKVDPSFFQRLEES | |
| | FLVEEDKSGSRHPIFGNIVDEVAYHEKYPTIYHLRKKLVDSDEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVDTSE | |
| | TKAPLSASMIKRYDEHHQDLTLLKELVRKYLPEKYKEIFFNQNNNGYAGYID | |
| | GGATQEEFYEYIKPILESMPGTKELLEKLEKRDLLRKQRTEDNGSIPHQIHL | |
| | GELKAILERQEKFYPFLKENREKIEKILSFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDELDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKKEVGKEKKLVEVKELLGITIMERSEFEKDPLGFLEKKGYKDVKKDKII | |
| | KLPKYSLFELGNGRKRMLASAGELQKGNELALPSEYVNFLYLASNYEKLKGD | |
| | PEEIKKKQEYVEKNKHYLDEIIEQISEFSKRVIKADANLEKVLEAYKKHKDK | |
| | PISEQAENIIHLFTLTALGAPAAFKYFDEVIDRKRYTSTKEVLDATLIHQSI | |
| | TGLYETRIDLKFLGGD | |
| CasEnd-228 | MKKPYSIGLDIGTNSVGWAVVTDDYKVPAKKMKVLGNTDKSHIKKNLLGALL | 188 |
| | FDSGNTAEDRRLKRTARRRYTRRRNRILYLQEIFSEEMGKVDDSFFHRLEDS | |
| | FLVPEDKRGERHPIFGNLEEEVKYHENFPTIYHLRKYLADNPEKADLRLVYL | |
| | ALAHIIKFRGHFLIEGKLDTRNNDVQRLFQEFLAVYDNTFEESSLQEQNVQV | |
| | EEILTDKISKSAKKDRVLKLFPNEKSNGRFAEFLKLIVGNQADFKKHFELEE | |
| | KAPLQFSKDTYEEDLETLLAQIGDDYADLFLSAKKLYDSILLSGILTVTDVS | |
| | TKAPLSASMIQRYNEHQMDLTQLKQFIRQKLPDKYNEVESDVSKDGYAGYID | |
| | GKTNQEDFYKYLKKLLNKIEGSGYFLDKIEREDFLRKQRTFDNGSIPHQIHL | |
| | QEMRAIIRRQAEFYPFLAENQDKIEKILTFRIPYYVGPLARGKSDFAWLSRK | |
| | SAEKITPWNFDEIVDKESSAEAFINRMTNYDLYLPNQKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASYGTYHDLRKILKDKDFLDNSKNEKILEDIVLTLT | |
| | LFEDREMIRKRLENYSDLLTKEQVKKLERRHYTGWGRLSAKLIHGIRNKESR | |
| | KTILDYLIDDGNSNRNFMQLINDDALSFKEEIAKAQVIGETDNLNQVVSDIA | |
| | GSPAIKKGILQSLKIVDELVKIMGGHQPENIVVEMARENQFTNQGRRNSQQR | |
| | LKGLTDSIKEFGSQILKEHPVENSQLQNDRLFLYYLQNGRDMYTGEELDIDY | |
| | LSQYDIDHIIPQAFIKDDSIDNRVLVSSKEARGKSDDVPSKDVVRKMKSYWS | |
| | KLLSAKLITQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLGKYPKLEPEFVYGDYPKFNSHKLFSKSKKEENKATAKKFFYS | |
| | NIMNFFKKDVKLADGSIVERPQIEVNDETGEIIWDKDKHISNVKKVLSYPQV | |
| | NIVKKVEEQTGGFSKESILPKGNSDKLIPRKTKWDTKKYGGFDSPIVAYSVL | |
| | VIADIEKGKSKKLKTVKELVGITIMEKMTFEKDPVAFLERKGYRNIQEENII | |
| | KLPKYSLFELENGRKRLLASARELQKGNEIVLPNHLVTLLYHAKNIHKVDEK | |
| | EEDIPKHLDYVDKHRDEFKELLDVVSNFSKKYTLAEGNLEKIKELYAQNNSA | |
| | DIKELASSFINLLTFTAIGAPATFKFFDKNIDRKRYTSTTEILNATLIHQSI | |
| | TGLYETRIDLSKLGGD | |
| CasEnd-229 | MKKKYSIGLDIGTNSVGWAVITDEYKVPSKKFKVLGNTDRHSIKKNLLGALL | 189 |
| | FDSGETAEATRLKRTARRRYTRRKNRIRYLQEIFAEEMSKVDDSFFHRLEES | |
| | FLVEEDKKHERHPIFGNIVDEVAYHEKYPTIYHLRKHLADSKEKADLRLIYL | |
| | ALAHIIKFRGHFLIEGDLNSENSDVQKLFIQLVQTYNQLFEENPINEETVDA | |
| | KAILSEKLSKSRRLENLIAQFPGEKKNGLFGNLIALSLGLTPNFKANFDLSE | |
| | DAKLQFSKDTYDEDLENLLAQIGDQYADLFLAAKNLSDAILLSDILTVNDES | |
| | TKAPLSASMIKRYDEHHQDLTLLKALVRQQLPEKYKEIFFDKSKNGYAGYID | |
| | GGASQEEFYKYIKPILEKMDGTEELLEKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKEASAQAFIERMTNFDKNLPTEKVLPKHSLLYEYFTV | |
| | YNELTKVKYVTEGMGKPEFLSGEQKKEIVDLLFKKNRKVTVKQLKEDYFKKI | |
| | ECLDSVEISGVEDRFNASLGTYHDLLKIIKDKEFLDNEENEDILEDIVLTLT | |
| | LFEDREMIEKRLKKYADLFDDKVLKQLKRRRYTGWGRLSAKLINGIRDKQSG | |
| | KTILDFLKDDGFANRNFMQLIHDDSLTFKEEIQKAQVEGQGDSLHEIIANLA | |
| | GSPAIKKGILQTVKIVDELVKVMGRHKPENIVIEMARENQTTQKGQKNSRER | |
| | LKRLEEVIKELGSQILKEHPVENTQLQNDKLYLYYLQNGKDMYTGEELDIDR | |
| | LSDYDVDHIIPQSFIKDDSIDNRVLTSSEEARGKSDDVPSEEVVKKMKSYWR | |
| | QLLNAKLISQRKFDNLTKAERGGLSELDKAGFIKRQLVETRQITKHVAQILD | |
| | SRFNTKRDENNKLIRDVKIITLKSKLVSDFRKDFQFYKVREINDYHHAHDAY | |
| | LNAVVGTALIKKYPKLESEFVYGDYKVYDSRKLIAKSEQEIGKATAKMFFYS | |
| | NIMNFFKTEIKLADGEIFKRPLIETNEETGEIVWDKEKDFATVRKVLSMPQV | |
| | NIVKKTEVQTGGFSKESILPKRDSDKLIARKKDWDPKKYGGFDSPTVAYSVL | |
| | VVAKIEKGKTKKLKTVKELVGITIMERSSFEKDPVAFLEAKGYQEIQEDLII | |
| | KLPKYSLFELENGRKRLLASAGELQKGNELALPQKYVTLLYLASHYEKLKGK | |
| | PEDEKQHKLYVEQHKSYFDEILDQISEFSERYILADKNLEKILELYKKNEDY | |
| | SISEQAENIINLETLTALGAPAAFKFFDTTIDRKRYTSTKEILDSTLIHQSI | |
| | TGLYETRIDLSQLGGD | |
| CasEnd-230 | MKKPYSIGLDIGTNSVGWAVVTDDYKVPAKKMKVLGNTDKSHIKKNLLGALL | 190 |
| | FDSGNTAEDRRLKRTARRRYTRRRNRILYLQEIFSEEMGKVDDSFFHRLEDS | |
| | FLVPEDKRGERHPIFGNLEEEVKYHENFPTIYHLRKHLADSPEKADLRLVYL | |
| | ALAHIIKFRGHFLIEGKFDTRNNDVQRLFQEFLAVYDNTFENSSLSEQNVQV | |
| | EEILTDKISKSAKKDRVLKLFPNEKSNGRFAEFLKLIVGNQADFKKHFELEE | |
| | KAPLQFSKDTYEEDLEVLLAQIGDEYADLFLSAKKLYDSILLSGILTVTDVS | |
| | TKAPLSASMIKRYNEHQMDLTQLKQFIRQKLPDKYNEVESDVSKDGYAGYID | |
| | GKTNQEDFYKYLKKLLNKIEGSEYFLDKIEREDFLRKQRTFDNGSIPHQIHL | |
| | QEMRAIIRRQAEFYPFLAENQDKIEKILTFRIPYYVGPLARGKSDFAWLSRK | |
| | SDEKITPWNFDEIVDKESSAEAFINRMTNYDLYLPNEKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASYGTYHDLLKILKDKDFLDNSKNEKILEDIVLTLT | |
| | LFEDREMIRKRLENYSDLLTKEQLKKLERRHYTGWGRLSAKLINGIRNKESR | |
| | KTILDYLIDDGNSNRNFMQLINDDALSFKEEIAKAQVIGETDNLNQVVSDIA | |
| | GSPAIKKGILQSLKIVDELVKIMGGHQPENIVVEMARENQFTNQGRRNSQQR | |
| | LKGLTDSIKEFGSQILKEHPVENSQLQNDRLFLYYLQNGRDMYTGEELDIDY | |
| | LSQYDIDHIIPQAFIKDDSIDNRVLTSSKEARGKSDDVPSKDVVRKMKSFWS | |
| | KLLSAKLITQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLGVYPQLEPEFVYGDYPKFNSHKLVKESTQEENKATAKKFFYS | |
| | NIMNFFKKDDKLADGSIVERPQIERNDENGEIIWKKDKHISNIKKVLSYPQV | |
| | NIVKKVEEQTGGFSKESILPKGNSDKLIPRKTKWDTKKYGGFDSPIVAYSVL | |
| | VIADIEKGKSKKLKTVKELVGITIMEKMTFEKDPVAFLERKGYQNIQEENII | |
| | KLPKYSLFELENGRKRLLASARELQKGNEIVLPNHLGTLLYHAKNIHKVDEK | |
| | NEEIPKHLDYVEKHRDEFKELLDVVSNFSKKYTLAEGNLEKIKELYAQNNQA | |
| | DIKELASSFINLLTFTAIGAPATFKFFDKNIDRKRYTSTTEILNATLIHQSI | |
| | TGLYETRIDLSKLGGD | |
| CasEnd-231 | MDKSYSIGLDIGTNSVGWAVITDEYKVPSKKFKVLGNTDRHSIKKNLIGALL | 191 |
| | FDSGETAEATRLKRTARRRYTRRKNRIRYLQEIFAEEMSKVDDSFFHRLEES | |
| | FLVEEDKRGERHPIFGNIVDEVAYHEKYPTIYHLRKKLADSTEKADLRLIYL | |
| | ALAHIIKFRGHFLIEGDLNSENSDVQKLFIQFVQTYDQTFEESHLSEETVDA | |
| | KAILTEKLSKSRRLENLIKQFPGEKKNGLFGNLIALSLGLTPNFKSNFGLAE | |
| | DAKLQFSKDTYDEDLENLLGQIGDQYADLFVAAKNLYDAILLSGILTVNTEI | |
| | TKAPLSASMIKRYDEHHQDLTLLKKFIREQLPEKYKEIFFDQSKNGYAGYID | |
| | GGASQEEFYKYIKPILSKMDGSEYFLEKIEREDFLRKQRTFDNGSIPHQIHL | |
| | GELHAILRRQEKFYPFLKENQEKIEKILTFRIPYYVGPLARGNSRFAWATRK | |
| | SDETITPWNFEEVVDKEASAQAFIERMTNNDKNLPEEKVLPKHSLLYEKFTV | |
| | YNELTKVKYITEGMRKPAFFSANQKEEIVDLLFKKNRKVTVKKLKEYLFKEF | |
| | ECFRSVEISGVEDRFNASLGTYHDLLKIIKDKDFLDNEENEDILEDIVLTLT | |
| | LFEDREMIEQRLSKYADLFDKKVLKKLKRRRYTGWGRLSAKLINGIRDKQSG | |
| | KTILDFLIDDGFANRNFMQLIHDDSLTFKEEIQKAQVIGQTDSLHEVIANLA | |
| | GSPAIKKGILQSIKIVDELVKVMGRHNPENIVIEMARENQTTQKGQRNSRER | |
| | LKRLEEGIKELGSKILKEHPVDNTQLQNDKLYLYYLQNGKDMYTGEELDIDR | |
| | LSDYDVDHIIPQSFIKDDSIDNRVLTSSAKARGKSDNVPSEEVVKKMKSFWR | |
| | QLLNAKLISQRKFDNLTKAERGGLTELDKAGFIKRQLVETRQITKHVAQILD | |
| | SRFNTKYDENDKLIRDVKIITLKSKLVSQFRKDFQFYKVREINDYHHAHDAY | |
| | LNAVVGTALIKKYPKLESEFVYGDYKVYNSRKMIAKSEQEIGKATAKYFFYS | |
| | NIMNFFKTDVTLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVADVEKGKAKKLKTVKELVGITIMERSSFEKNPVAFLEDKGYQNIQKDLII | |
| | KLPKYSLFELENGRKRLLASAGELQKGNELALPQKYVTLLYHAKHYEKLKES | |
| | PEDNEKKKEYVEQHRQEFDEILDQIGEFSERYILADKNLEKIKELYAENEDA | |
| | SIEELASSFINLLTFTALGAPAAFKFFDKTIDRKRYTSTTEILNATLIHQSI | |
| | TGLYETRIDLSQLGGD | |
| CasEnd-232 | MKKKYSIGLDIGTNSVGWAVVTDDYKVPSKKMKVLGNTDRQSIKKNLLGALL | 192 |
| | FDSGETAEATRLKRTARRRYTRRKNRILYLQEIFAEEMNKVDDSFFHRLEES | |
| | FLVEEDKRGERHPIFGNIVEEVAYHEKFPTIYHLRKHLADSTEKADLRLVYL | |
| | ALAHIIKFRGHFLIEGDLNTENTDVQKLFLQFVETYDNLFEESPLGEETVDA | |
| | ESILTAKLSKSRRLENLIKQFPNEKKNGLFGNLIALSLGLQPNFKSNFGLAE | |
| | DAKLQFSKDTYDEDLENLLGQIGDEYADLFAAAKNLYDAILLSGILTVNDES | |
| | TKAPLSASMIKRYEEHHEDLTLLKYFIRNNLPEKYKEIFFDESKNGYAGYID | |
| | GGVKQEEFYKYLKNLLSKLDGSEYFLDKIEREDFLRKQRTFDNGSIPHQIHL | |
| | QELHAILRRQAEYYPFLKENQEKIEKILTFRIPYYVGPLARGNSRFAWMSRK | |
| | SDETITPWNFDEVVDKEASAEAFIERMTNNDLYLPNEKVLPKHSLLYEKFTV | |
| | YNELTKVKYVTEGMGKAQFFSAEQKKEIFDGLFKKNRKVTKKKLKNFLDKEF | |
| | DEFRIVDISGVEDAFNASLGTYHDLLKIIKDKEFLDNEENEDILEDIVLTLT | |
| | LFEDREMIRQRLSKYADLFDKKVLKKLERRHYTGWGRLSAKLINGIRDKQTG | |
| | KTILDYLIDDGFANRNFMQLIHDDSLSFKEEIEKAQVIGDTDSLHEVVANLA | |
| | GSPAIKKGILQSLKIVDELVKVMGRHNPENIVIEMARENQTTAKGQRNSRER | |
| | LKRLEEAIKKLGSNILKEHPVENQQLQNDRLYLYYLQNGKDMYTGEELDIDR | |
| | LSQYDVDHIIPQSFIKDDSIDNRVLTSSKKARGKSDNVPSEEVVRKMKSYWM | |
| | QLLDAKLISQRKFDNLTKAERGGLTEDDKAGFIKRQLVETRQITKHVAQILD | |
| | SRFNTERDENDKLIRKVKIITLKSKLVSDFRKDFGLYKVREINDYHHAHDAY | |
| | LNAVVGKALIKKYPKLEPEFVYGDYPKYNVRKMVRKSDQEIGKATAKRFFYS | |
| | NIMNFFKSEIKLADGRIVERPQIEANEETGEIAWDKEKDFATVRKVLSMPQV | |
| | NIVKKTEVQTGGFSKESILPKRDSDKLIARKKKWDPKKYGGFDSPTVAYSVL | |
| | VVADIEKGKAKKLKTVKELVGITIMERSAFEKNPVAFLENKGYQNIQKDLII | |
| | KLPKYSLFELENGRKRLLASAGELQKGNELALPQKLVTLLYHAKHYEKLKEK | |
| | EEDNEKHMEYVEQHRDEFKEIFDQISEFSERYILADKNLEKISSLYAKNEDA | |
| | SIEELASSFINLLTFTALGAPAAFKFFGTTIDRKRYTSTTEILNATLIHQSI | |
| | TGLYETRIDLSKLGGD | |
| CasEnd-233 | MDKKYSIGLDIGTNSVGWAVVTDEYKVPSKKFKVLGNTDRKSIKKNLLGALL | 193 |
| | FDSGETAEATRLKRTARRRYTRRKNRICYLQEIFSNEMAKVDDNFFQRLEES | |
| | FLVEEDKKNDRHPIFGNIVEEVAYHEKYPTIYHLRKKLADSTEKADLRLVYL | |
| | ALAHIIKFRGHFLIEGDLNSENTDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNDES | |
| | TKAPLSASMVKRYDEHHQDLTLLKQFVRKQLPEKYKEIFFDQSKNGYAGYID | |
| | GGASQEEFYKYLKKILEKIDGSEEFLDKIEREDFLRKQRTFDNGSIPHQIHL | |
| | QELHAIIRRQEKYYPFLKENKEKIEKILTFRIPYYVGPLARGNSRFAWLSRK | |
| | SDETITPWNFDEVVDKEASAEAFIERMTNFDKNLPDEKVLPKHSLLYEKFTV | |
| | YNELTKVKYVTEGMRKPEFFSAEQKQEIVDLLFKKNRKVTKKQLKEYLVKEF | |
| | DEFDIVEISGVEDRFNASLGTYHDLLKIIKDKDFLDNEENEKILEDIILTLT | |
| | LFEDREMIKKRLEKYADLFDKKQLKKLKRRRYTGWGRLSAKLINGIRDKQTG | |
| | KTILDYLIDDGFANRNFMQLIHDDSLTFKEEIEKAQVIGKSESLHETIANLA | |
| | GSPAIKKGILQSLKIVDELVKVMGRHAPENIVVEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENGKLIRDVKIITLKSKLVSDFRKDFELYKVREINNYHHAHDAY | |
| | LNAVVGTALIKKYPKLESEFVYGDYKVYDVRKMIAKSEKEIGKATAKYFFYS | |
| | NIMNFFKSEVTLANGTIRKRPLIEVNEETGEIVWDKEKDIATVKKVLSMPQV | |
| | NIVKKTEVQTGGFSKESILPKRDSDKLIARKKDWDTKKYGGFDSPTVAYSVL | |
| | VVADVEKGKAKKLKTVKELVGITIMERSAFEKNPIAFLEDKGYKNIQKDNII | |
| | KLPKYSLFELENGRKRLLASAGELQKGNELALPQKYVTLLYHAKHYEKLKGS | |
| | PEDNEKHQYYVEEHKDEFDEILDQIIEFSKRYILADANLEKIKKLYEKNEDA | |
| | SIEELAENFIHLLTFTALGAPAAFKFFGTTIDRKRYTSTKEILNATLIHQSI | |
| | TGLYETRIDLSKLGGD | |
| CasEnd-234 | MDKKYSIGLDIGTNSVGWAVITDEYKVPSKKFKVLGNTDRHSIKKNLIGALL | 194 |
| | FDSGETAEATRLKRTARRRYTRRKNRICYLQEIFSNEMAKVDENFFQRLDDS | |
| | FLYPEDKRNDKYPIFGTLAEEKDYHKQFPTIYHLRKELADNDEKADLRLVYL | |
| | ALAHIIKYRGHFLIEGNLDSENTDIQATFKDFIEVEDRTVENSSLSEETVDV | |
| | ESILTEKISKSRRLEKLLKKFPTEKKNTIFAEFLKLIVGNTADFKKNFGLEE | |
| | DAKLQFSKDTYEEDLEELLGKIGDEYADLFIAAKKLYDAILLSGILTGKDNS | |
| | TKAPLSASMVDRYEEHQKDLKKLKEFIKKNFPDEYNEIFRDKTKNGYAGYIE | |
| | GKTKQDDFYKYLKKLLSKIEGSDYFLDKIEREDFLRKQRTFDNGSIPHQVHL | |
| | QEMKAIIRRQGKYYPFLKENQDKIEKILTFRIPYYVGPLARKKSRFAWAERK | |
| | TDEKITPWNFDDVIDKEKSAEKFITRMTNNDLYLPEEKVLPKHSLLYEKFTV | |
| | YNELTKVKYINEQGKEEKFFDANMKQEIFENVFKKYRKVTKKKLLDYLVKEF | |
| | DELRIVDLTGLDKRFNSSLGTYHDLKKILFDKSFLDDDANQEMIEDIIQTLT | |
| | LFEDKEMIKKRLEKYSDILTKEQLKKLEKRHYTGWGRLSAKLINGIRNKETG | |
| | KTILDYLIDDGYTNRNFMQLIHDDTLSFKDIIAEAQAIKDVDSLHEHIANLA | |
| | GSPAIKKGILQTVKVVDELVKVMGRHKPENIVIEMARENQTTQKGQRRSQQR | |
| | LKKLQESLKELKKDILKEYPTDNQKLQSDRLFLYYIQNGKDMYTGEPLDIDN | |
| | LSQYDIDHIIPQAFIKDDSIDNRVLVSSAEARGKSDDVPSIDIVNKMKSFWK | |
| | RLLEAGLISQRKYDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-235 | MDKKYSIGLDIGTNSVGWAVITDEYKVPSKKFKVLGNTDRQSIKKNLLGALL | 195 |
| | FDSGETAEATRLKRTARRRYTRRKNRIRYLQEIFAEEMSKVDDSFFHRLEES | |
| | FLVEEDKRKERHPIFGNIVEEVAYHEKYPTIYHLRKKLADSTEKADLRLIYL | |
| | ALAHIIKFRGHFLIEGDLNSENSDVQKLFIQLVQTYNQLFEENPISEEGVDA | |
| | KAILSAKLSKSRRLENLIALFPGEKKNGLFGNLIALSLGLTPNFKSNFDLSE | |
| | DAKLQLSKDTYDEDLDNLLAQIGDQYADLFLAAKNLSDAILLSGILTVNDEI | |
| | TKAPLSASMIKRYDEHHQDLTLLKALVRQQLPEKYKEIFFDESKNGYAGYID | |
| | GGASQEDFYKFIKPILEKLDGTEELLAKIEREDLLRKQRTFDNGSIPHQIHL | |
| | NELKAILRRQEEFYPFLKENREKIEKILTFRIPYYVGPLARGNSRFAWMTRK | |
| | SEETITPWNFEEVVDKGASAQAFIERMTNFDKNLPEEKVLPKHSLLYEYFTV | |
| | YNELTKVKYVTEGMRKPEFLSGEQKEAIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLSKYANLFDKKVLKQLKRRRYTGWGRLSRKLINGIRDKQSG | |
| | KTILDFLKDDGFANRNFMQLIHDDSLTFKEEIQKAQVVGQGDSLHEQIANLA | |
| | GSPAIKKGILQSLKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIRRVKIITLKSKLVSDFRKDFQLYKVREINNYHHAHDAY | |
| | LNAVVGTALIKKYPKLESEFVYGDYKVYDVRKMIAKSEQEIGKATAKYFFYS | |
| | NIMNFFKTEITLANGTIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKTKKLKTVKELVGITIMERSSFEKDPVAFLEDKGYKDIQEDLII | |
| | KLPKYSLFELENGRKRLLASAGELQKGNELALPQKYVTLLYHAKRYEKLKGK | |
| | PEDREQKLEYVEKHRHEFKEIFDQISEFAERYILADANLEKVLELYSKFEDA | |
| | PIEELAENFIHLFTFTSLGAPAAFKFFDTTIDRKRYTSTKEILNATLIHQSI | |
| | TGLYETRIDLSKLGGD | |
| CasEnd-236 | MDKPYSIGLDIGTNSVGWAVITDEYKVPSKKFKVLGNTDRHSIKKNLLGALL | 196 |
| | FDSGETAEATRLKRTARRRYTRRKNRILYLQEIFSEEMSKVDDSFFHRLEES | |
| | FLVEEDKRGERHPIFGNIVDEVAYHEKYPTIYHLRKELADSTEKADLRLIYL | |
| | ALAHMIKFRGHFLIEGQLNPENTDVQKLFQAFVEVYNRTFEESHLQEETVDV | |
| | EAILTEKVSKSRRLENLIKQFPGEKKNGLFGNLIALSLGLTPNFKSNEDLSE | |
| | DAKLQFSKDTYDEDLENLLGQIGDEYADLFLAAKNLSDAILLSGILTVNTEI | |
| | TKAPLSASMIKRYDEHHQDLTLLKKFVRQQLPEKYKEIFFDKSKNGYAGYID | |
| | GGASQEDFYKYLKPILSKLDGSEYFLDKIEREDFLRKQRTFDNGSIPHQIHL | |
| | QELHAILRRQEVYYPFLKENQEKIEKILTFRIPYYVGPLARGNSRFAWMTRK | |
| | SDETITPWNFEEVVDKEKSAEAFIERMTNNDKNLPEEKVLPKHSLLYEKFTV | |
| | YNELTKVKYVTEGMRKPAFLDSNQKQEIFDLLFKKNRKVTVKKLKEFLFKKF | |
| | EEFDIVEISGVEKRFNASLGTYHDLLKIIKDKDFLDNPENEEILEDIVLTLT | |
| | LFEDREMIEQRLAKYADLFDKKVLKKLKRRRYTGWGRLSAKLINGIRDKQSG | |
| | KTILDYLIDDGFANRNFMQLIHDDSLSFKEEIKKAQVIGKTDSLHEVIAELA | |
| | GSPAIKKGILQSIKIVDELVKVMGRYAPENIVIEMARENQTTQKGQKNSRER | |
| | LKRLEESIKKLGSNILKEHPVDNTQLQNDKLYLYYLQNGKDMYTGEELDINR | |
| | LSDYDVDHIIPQSFIKDDSIDNRVLVSSKKARGKSDNVPSEEVVKKMKSFWY | |
| | QLLKAKLISQRKFDNLTKAERGGLTELDKAGFIKRQLVETRQITKHVAQILD | |
| | SRMNTKYDENDKLIRKVKIITLKSKLVSDFRKEFGFYKVREINDYHHAHDAY | |
| | LNAVVGTALIKKYPKLESEFVYGDYKVYNSRKMIAKSEQEIGKATAKYFFYS | |
| | NIMNFFKTDITLANGEIRKRPLIETNKETGEIVWDKEKDFATVKKVLSMPQV | |
| | NIVKKTEVQTGGFSKESILPKRNSDKLIARKKDWDTKKYGGFDSPTVAYSVL | |
| | VVADIEKGKAKKLKTVKELVGITIMERSAFEKNPIVFLENKGYKNIQKDLII | |
| | KLPKYSLFELENGRKRLLASAGELQKGNELALPNKYVTLLYLAKHYEKLKGS | |
| | PEDNEKHLEYVEQHLSEFDEILNQISEFAKRYILADANLEKIQELYTQNEDA | |
| | SIEELAESFINLLTFTALGAPAAFKFFGKTIDRKRYTSTKEILNSTLIHQSI | |
| | TGLYETRIDLSQLGGD | |
| CasEnd-237 | MDKKYSIGLDIGTNSVGWAVITDEYKVPSKKFKVLGNTDRHSIKKNLLGALL | 197 |
| | FDSGETAEATRLKRTARRRYTRRKNRILYLQEIFAEEMNKVDDSFFHRLEES | |
| | FLVEEDKRGERHPIFGNIVEEVAYHEKYPTIYHLRKHLADSTEKADLRLVYL | |
| | ALAHIIKFRGHFLIEGDLNAENTDVQKLFIDFVETYDRTFEESHLSEITVDA | |
| | SEILTDKISKSRKLENLIKLFPNEKKNGLFGNLIALILGNQPNFKINFELSE | |
| | DAKLQFSKDTYEEDLENLLGQIGDEYADLFVAAKNLYDAILLSGILTVNTEI | |
| | TKAPLSASMIKRYDEHHQDLTLLKAFIRKNLPEKYKEIFFDESKNGYAGYID | |
| | GGASQEEFYKYIKPLLSKIEGSEYFLDKIEREDFLRKQRTFDNGSIPHQIHL | |
| | QELHAILRRQEEYYPFLKENQEKIEKILTFRIPYYVGPLARGNSRFAWASRK | |
| | SDETITPWNFDEVVDKEKSAEAFIERMTNNDKNLPEEKVLPKHSLLYEKFTV | |
| | YNELTKVKYVTEGMRKPAFFDANLKQEIFDLLFKENRKVTKKKLLDFLDKEF | |
| | DEFRIVDISGVEKSFNASLGTYHDLLKIIKDKEFLDNPENEEILEDIVLTLT | |
| | LFEDREMIKQRLSKYADLFDKKQLKKLKRRRYTGWGRLSAKLINGIRDKQSG | |
| | KTILDYLIDDGFANRNFMQLIHDDSLSFKEEIAKAQVIGESDSLHQVIADLA | |
| | GSPAIKKGILQSIKIVDELVKVMGRYNPENIVIEMARENQTTQKGQRNSRER | |
| | LKRLEESLKELGSKILKEHPVDNTQLQNDKLYLYYLQNGRDMYTGEELDIDR | |
| | LSDYDVDHIIPQSFIKDDSIDNRVLTSSAKARGKSDDVPSEEVVKKMKSFWK | |
| | KLLDAKLISQRKFDNLTKAERGGLTELDKAGFIKRQLVETRQITKHVAQILD | |
| | SRFNTKYDENDKLIRDVKIITLKSKLVSQFRKEFELYKVREINDYHHAHDAY | |
| | LNAVVGKALIKKYPKLESEFVYGDYPVYNSYKMIAKSEQEIGKATAKYFFYS | |
| | NIMNFFKTDITLANGEIRKRPLIETNEETGEIAWDKEKHIATVKKVLSMPQV | |
| | NIVKKTEVQTGGFSKESILPKRNSDKLIARKKDWDTKKYGGFDSPTVAYSVL | |
| | VIADIEKGKAKKLKTVKELVGITIMERSSFEKNPIAFLEDKGYQNIQKDNII | |
| | KLPKYSLFELENGRKRLLASAGELQKGNELALPNHYVTLLYHAKHYEKLKES | |
| | PEDNPKHLNYVEEHRSEFDELLDQISEFSKRYILADKNLEKIKELYAKNKDA | |
| | DIEELASSFINLLTFTALGAPAAFKFFGKDIDRKRYTSTTEILNATLIHQSI | |
| | TGLYETRIDLSQLGGD | |
| CasEnd-238 | MDKKYSIGLDIGTNSVGWAVITDEYKVPSKKFKVLGNTDRHSIKKNLIGALL | 198 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKELVRKQLPEKYKEIFFDQSKNGYAGYID | |
| | GGASQEDFYKYIKKILEKMDGTEELLAKLEREDLLRKQRTFDNGSIPHQIHL | |
| | GELHAILRRQEEYYPFLKDNREKIEKILTFRIPYYVGPLARGNSRFAWMKRK | |
| | SEETITPWNFDEVVDKGASAQAFIERMTNFDKNLPNEKVLPKHSLLYEYFTV | |
| | YNELTKVKYVTEGMRKPEFLDGEQKKEIVDNLFKKNRKVTVKQLKEYYFKKE | |
| | DCFDSVEISGVEDRFNASLGTYHDLLKIIKDKAFLDNEENEEILEDIVLTLT | |
| | LFEDREMIKERLEKYADLFDDKVMKQLKRRRYTGWGRLSRKLINGIRDKQSG | |
| | KTILDFLIDDGFTNRNFMQLIHDDSLTFKEEIEKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKIEKGKTKKLKTVKELLGITIMERSAFEKNPIAFLENKGYQNVKKDLII | |
| | KLPKYSLFELENGRKRMLASAGELQKGNELALPSKYVNFLYLASNYEKLKGS | |
| | PEDNKRKQLFVEQHKDYLDEIIDQISEFSKRVILADANLEKVKKAYEKHKNK | |
| | SIEEQAENIIHLFTLTALGAPAAFKYFDKDIDRKRYTSTKEVLNATLIHQSI | |
| | TGLYETRIDLSQLGGD | |
| CasEnd-239 | MDKKYSIGLDIGTNSVGWAVVTDEYKVPSKKFKVLGNTDRKSIKKNLIGALL | 199 |
| | FDSGETAEATRLKRTARRRYTRRKNRIRYLQEIFAEEMAKVDDSFFHRLEES | |
| | FLVEEDKKNERHPIFGNIVDEVAYHEKYPTIYHLRKHLADSTEKADLRLIYL | |
| | ALAHIIKFRGHFLIEGDLNSENSDVQKLFEQLVQTYNQLFEESPLDEEEVDA | |
| | EAILTEKLSKSRRLENLIALFPGEKKNGLFGNLIALSLGLTPNFKSNFDLSE | |
| | DAKLQFSKDTYDEDLEELLGQIGDEYADLFVAAKNLYDAILLSGILTVNDES | |
| | TKAPLSASMVKRYDEHHQDLTLLKQFIRKQLPEKYKEIFFDQSKNGYAGYID | |
| | GGASQEEFYKYLKKILEKIDGSEEFLDKIEREDFLRKQRTFDNGSIPHQIHL | |
| | QELHAILRRQEKYYPFLKENQEKIEKILTFRIPYYVGPLARGNSRFAWMSRK | |
| | SDETITPWNFEEVVDKEKSAEKFIERMTNFDKNLPNEKVLPKHSLLYEKFTV | |
| | YNELTKVKYVTEGMKKPEFFDANQKQEIVDLLFKKNRKVTKKQLKEYLFKEF | |
| | DEFDIVEISGVEDRFNASLGTYHDLLKIIDDKDELDNEENEDILEDIILTLT | |
| | LFEDREMIKKRLKKYADLFDKKQLKKLKRRRYTGWGRLSAKLINGIRDKQTG | |
| | KTILDYLIDDGFANRNFMQLIHDDSLTFKEEIEKAQVIGDGESLHEVIANLA | |
| | GSPAIKKGILQSLKIVDELVKVMGRHEPENIVIEMARENQTTQKGQKNSRER | |
| | MKRLEEAIKELGSKILKEHPVENTQLQNDKLYLYYLQNGKDMYTGEELDIDR | |
| | LSDYDVDHIVPQSFLKDDSIDNKVLVSSAKARGKSDDVPSEEIVKKMKSYWK | |
| | KLLDAKLITQRKFDNLTKAERGGLTELDKAGFIKRQLVETRQITKHVAQILD | |
| | SRFNTKYDENDKLIRDVKIITLKSKLVSQFRKDFGLYKVREINNYHHAHDAY | |
| | LNAVVGTALIKKYPKLESEFVYGDYKVYDVRKMIAKSEKEIGKATAKYFFYS | |
| | NIMNFFKTEVKLANGEIRKRPLIEVNEETGEIVWDKEKDFATVRKVLSMPQV | |
| | NIVKKTEVQTGGFSKESILPKRDSDKLIARKKDWDTKKYGGFDSPTVAYSVL | |
| | VVAKIEKGKAKKLKTVKELVGITIMERSAFEKNPVAFLEAKGYQEVQKDLII | |
| | KLPKYSLFELENGRKRLLASAGELQKGNELALPQKYVILLYHAKHYEKLKGS | |
| | PEDNEEKLLYVEQHKEYFDEIIEQISEFAKRYILADANLEKIKELYEKNRDA | |
| | DIEELAESFINLLTFTALGAPAAFKFFDTTIDRKRYTSTKEILNATLIHQSI | |
| | TGLYETRIDLSKLGGD | |
| CasEnd-240 | MKKPYSIGLDIGTNSVGWAVVTDDYKVPAKKMKVLGNTDKSHIKKNLLGALL | 200 |
| | FDSGNTAEGRRLKRTARRRYTRRRNRILYLQEIFSEEMNKVDESFFHRLDDS | |
| | FLVPEDKRGERHPIFGDLAEETKYHKEFPTIYHLRKHLADSPEKADLRLVYL | |
| | ALAHIIKYRGHFLIEGDLDTRNNDVQQLFQEFLAIYDNTFERSSLQEQNAQA | |
| | EEILTDKISKSAKKERVLKLFPNEKSNGFFAEFLKLIVGNQADFKKNFELEE | |
| | KAPLQFSKDSYEEDLETLLGQIGDEYADLFVAAKKLYDSILLSGILTVTDVS | |
| | TKAPLSASMIQRYEEHNMDLAKLKDFIRKNLSHKYKEVENDESKDGYAGYID | |
| | GKTTQEAFYKYLKKLLSKTEGSGYFLDKIEREDFLRKQRTFDNGSIPHQIHL | |
| | QEMRAIIRRQAEFYPFLAENQEKIEQILTFRIPYYVGPLARGESDFAWASRK | |
| | SDEKITPWNEDDIIDKESSAEAFINRMTNYDLYLPEEKVLPKHSLLYEKFTV | |
| | YNELTKVKYITEQMGKTQFFDANLKQEIFDGVFKVERKVTKKKLMDFLHHEF | |
| | DEFRIVDLTGIDKAFNASLGTYHDLLKILNDKEFLDDSENEAILEDIVLTLT | |
| | LFEDREMIKQRLSKYSDLFTKEQLKKLERRHYTGWGRLSAKLINGIRDKHTR | |
| | KTILDYLIDDGRSNRNFMQLINDDALSFKEEIAKAQVIGETDNLKQVVQDLA | |
| | GSPAIKKGILQSLKIVDELVKIMGGYNPENIVVEMARENQFTNRGRRNSQQR | |
| | LKGLTDSIKELGSKILKEHPVDNSQLQNDRLFLYYLQNGKDMYTGEALDIDY | |
| | LSQYDIDHIIPQAFIKDDSLDNRVLVSSAKARGKSDDVPSKEVVQKMKSFWS | |
| | KLLDSKLISQRKFDNLTKAERGGLIDDDKAGFIKRQLVETRQITKHVARILD | |
| | ERFNTETDENNKKIRSVKIVILKSNLVSNFRKEFEFYKVREINDYHHAHDAY | |
| | LNAVVAKALLKKYPKLEPEFVYGEYPKYNSYRIVVENVKERKSATAKMFFYS | |
| | NIMNFFKKTIKLADGTVVERPMIEVNEETGEIVWDKTKHISTVKKVLSYPQV | |
| | NIVKKVEEQTGGFSKESILPKGDSDKLIPRKTKWDTKKYGGFDSPIVAYSVL | |
| | VIADIEKGKAKKLKTVKELVGITIMERATFEKDPVAFLERKGYQNIQKENII | |
| | KLPKYSLFELENGRRRLLASAKELQKGNEMVLPNHLVILLYHAKHIHKVDEK | |
| | SEDAPKHLQYVDKHRSEFKELLDVVSNFSKKYILAEKNLEKIDELFDQNNGA | |
| | SVEELASSFINLLTFTAIGAPATFKFFGKNIDRKRYTSTTEILNATLIHQSI | |
| | TGLYETRIDLSKLGED | |
| CasEnd-241 | MDKKYSIGLDIGTNSVGWAVITDEYKVPSKKFKVLGNTDRHSIKKNLIGALL | 201 |
| | FDSGETAEATRLKRTARRRYTRRKNRICYLQEIFSNEMAKVDENFFQRLDDS | |
| | FYVPEDKRGDKYPIFGTLKEEKDYHKEFPTIYHLRKHLADSDEKADLRLVYL | |
| | ALAHIIKYRGHFLIEGELDSRNTDIQKTFKDFLEIFDRTFEESHLQEELIDV | |
| | ESILTEKISKSRRVEKLLKKFPNQKKNTIFAEFLKLIVGNTADFKKVENLEE | |
| | DAKLQFSKETYDEDLEELLGEIGDEYADLFSSAKKLYDAILLSGILTGKDNS | |
| | TKAPLSASMVQRYEEHKEDLKKLKKFIKKNAPEKYNEIFKDKAKNGYAGYIE | |
| | NKTKQEDFYKYLKKLLTKVEGSDYFLDKIEREDFLRKQRTFDNGVIPHQVHL | |
| | QELKAIIRNQEKYYPFLKENQDKIEKILTFRIPYYVGPLARKKSRFAWAERK | |
| | SDEKITPWNFDDVIDKEKSAEKFITRMTNNDLYLPEEKVLPKHSLLYEKFTV | |
| | YNELTKVRYINEQGKEEKFFDANLKQEIFNDVFKKERKVTKKKLLDYLEKEF | |
| | DELRIVDITGLDKRFNSSLGTYHDLKKILFDKSFLDDPDNQEMIEEIIQTLT | |
| | LFEDKKMIKKRLEKYSDILTKSQIKKLEKRHYTGWGRLSAKLINGIRDKETG | |
| | KTIMDYLIDDGYTNRNFMQLIHDDNLSFKDIISEAQIIKDEDSLHEHIANLA | |
| | GSPAIKKGILQTVKVVDELVKVMGRHKPENIVIEMARENQTTQKGQRRSQQR | |
| | LKLLQDSVKNLASKILKEYPTDNQKLQSDRLFLYYLQNGKDMYTGEPLDIDN | |
| | LSQYDIDHIIPQAFIKDDSIDNRVLVSSAEARGKSDDVPSIEIVNKMKGFWK | |
| | KLLDAGLISKRKYDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-242 | MDKPYSIGLDIGTNSVGWAVITDEYKVPSKKFKVLGNTDRHSIKKNLIGALL | 202 |
| | FDSGETAEATRLKRTARRRYTRRKNRICYLQEIFAEEMNKVDDSFFHRLEES | |
| | FLVEEDKRYERHPIFGNIVDEVAYHEKYPTIYHLRKELADSDEKADLRLIYL | |
| | ALAHIIKFRGHFLIEGDLSSENTDVQKLFLQFVQTYNQLFEESNLNEETVDA | |
| | EAILTAKMSKSRRLENLIAQFPAEKKNGLFGNLVALSLGLTPNFKSNFELTE | |
| | DAKLQFSKDTYDEDLENLLAQIGDQYADLFLAAKNLYDAILLSGILTVNTEI | |
| | TKAPLSASMIKRYDEHHQDLTLLKVFVRDQLPEKYKEIFFDDTKNGYAGYID | |
| | GGASQEEFYKYIKPILIKIDGSEELLEKIEREDFLRKQRTFDNGSIPHQIHL | |
| | QELHAILRRQGKYYPFLKENQEKIEKILTFRIPYYVGPLARGNSRFAWMTRK | |
| | SNETITPWNFEEVVDKEASAQAFIERMTNFDKNLPCEKVLPKHSLLYEKFTV | |
| | YNELTKVKYITEGMRKPAFLSSNQKKEIVDLLFKKNRKVTVKQLKEFLTKKI | |
| | ECFDSVEISGVEDKFNASLGTYHDLLKIIKDKDFLDNEENEDILEDIVLTLT | |
| | LFEDREMIEKRLSKYADLFDKKVLKKLKRRRYTGWGRLSAKLINGIRDKQSG | |
| | KTILDYLIDDGFANRNFMQLIHDDSLTFKEEIAKAQVIGNSDSLHETIANLA | |
| | GSPAIKKGILQSIKIVDELVKVMGRHNPENIVIEMARENQTTQKGQKNSRER | |
| | LKRLEEAIKELGSQILKEHPVDNTQLQNDKLYLYYLQNGKDMYTGEELDIDR | |
| | LSDYDVDHIIPQSFIKDDSIDNRVLVSSAKARGKSDNVPSEEVVKKMKNEWS | |
| | KLLNAKLISQRKFDNLTKAERGGLSELDKAGFIKRQLVETRQITKHVAQILD | |
| | SRFNTKYDENDKLIRKVKIITLKSKLVSDFRKDFGLYKVREINNYHHAHDAY | |
| | LNAVVGKALIKKYPKLESEFVYGDYPVYNSAKMIAKSEQEIGKATAKYFFYS | |
| | NIMNFFKTEITLANGEIRKRPLIETNEETGEIVWDKEKHFATVRKVLSMPQV | |
| | NIVKKTEVQTGGFSKESILPKRDSDKLIARKKDWDTKKYGGFDSPTVAYSVL | |
| | VIADIEKGKTKKLKTVKELVGITIMERSAFEKDPIAFLEDKGYQNIQKEKII | |
| | KLPKYSLFELENGRKRLLASAGELQKGNELALPGKYVTLLYHAKHYEKLKES | |
| | PEDNEKHKYYVEQHRDEFDEILEQISEFSERYILADSNLEKIRELYDKNSNK | |
| | SISELAESFINLLTFTAFGAPAAFKFFGQTIDRKRYTSTTEILNATLIHQSI | |
| | TGLYETRIDLSQLGGD | |
| CasEnd-243 | MKKPYSIGLDIGTNSVGWAVVTDDYKVPAKKMKVLGNTDKSHIKKNLLGALL | 203 |
| | FDSGNTAEDRRLKRTARRRYTRRRNRILYLQEIFSEEMGKVDDSFFHRLEDS | |
| | FLVPEDKRGERHPIFGNLEEEVKYHENFPTIYHLRKYLADSPEKADLRLVYL | |
| | ALAHIIKFRGHFLIEGKLDTRNNDVQRLFQEFLEVYDNTFERSSLQEQNVQV | |
| | EEILTDKISKSAKKDRILKLFPNEKSNGRFAEFLKLIVGNQADFKKHFELEE | |
| | KAPLQFSKDTYEEDLENLLAQIGDEYADLFLSAKKLYDSILLSGILTVTDVS | |
| | TKAPLSASMIQRYKEHQMDLTQLKQFIRQKLSDKYNEVESDVSKDGYAGYID | |
| | GKTTQEAFYKYLKGLLNKIEGSEYFLDKIEREDFLRKQRTFDNGSIPHQIHL | |
| | QEMRAIIRRQAEFYPFLAENQDKIEKILTFRIPYYVGPLARGKSDFAWLSRK | |
| | SAEKITPWNFDEIVDKESSAEAFINRMTNYDLYLPNEKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASLGTYHDLRKILNDKDFLDNSKNEKILEDIVLTLT | |
| | LFEDREMIRKRLENYSDLLTKEQLKKLERRHYTGWGRLSAKLINGIRNKESR | |
| | KTILDYLIDDGNSNRNFMQLINDDALSFKEEIAKAQVIGETDNLNQVVSDLA | |
| | GSPAIKKGILQSLKIVDELVKIMGNHNPENIVVEMARENQFTNQGRRNSQQR | |
| | LKGLTDSIKEFGSQILKEHPVENSQLQNDRLFLYYLQNGRDMYTGEELDIDY | |
| | LSQYDIDHIIPQAFIKDDSIDNRVLVSSAEARGKSDDVPSKDVVRKMKSYWS | |
| | KLLSAKLITQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLKKYPKLEPEFVYGEYPKFNSYKFVAKTKEEENKATAKMFFYS | |
| | NIMNFFKKDVKLADGSIVERPVVEVNDETGEIIWDKDKHISTIKKVLSYPQV | |
| | NIVKKVEEQTGGFSKESILPKGNSDKLIPRKTKWDTKKYGGFDSPIVAYSVL | |
| | VIADIEKGKSKKLKTVKELVGITIMEKMTFEKDPVAFLERKGYQNIQEENII | |
| | KLPKYSLFELENGRKRLLASAKELQKGNEIVLPNHLVTLLYHAKNIHKVDEK | |
| | EEEIPKHLEYVDKHKDEFKELLDVVSNFSKKYTLAEKNLEKIKELYAQNNSA | |
| | DIKELASSFINLLTFTAIGAPATFKFFDKNIDRKRYTSTTEILNATLIHQSI | |
| | TGLYETRIDLSKLGGD | |
| CasEnd-244 | MKKPYSIGLDIGTNSVGWAVVTDDYKVPSKKMKVLGNTDRQSIKKNLLGALL | 204 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEQVADLA | |
| | GSPAIKKGILQSLKIVDEIVKVMGRYAPQNIVVEMARENQTTAKGQRNSRER | |
| | LKRLEEALKKLGSKILKEHPVENSQLQSDRLYLYYLQNGKDMYTGEELDIDR | |
| | LSQYDVDHIIPQSFIKDDSIDNRVLTSSKEARGKSDDVPSEDVVRKMKPYWS | |
| | KLLRSNLISQRKFDNLTKAERGGLTQDDKAGFIKRQLVETRQITKHVAQILD | |
| | SRFNKEFDDNNKLIREVKIVTLKSKLVSQFRKEFGLYKVREINDYHHAHDAY | |
| | LNAVVGKALIKKYPKLEPEFVYGDYPKYNSHKLIGKSDKERGKATAKMFFYS | |
| | NIMNFFKSDVKLADGTIFERPPIEVNEETGEIVWDKTKHIATVKKVLSMPQV | |
| | NIVKKTEVQTGGFSKESILPKRESDKLIARKKGWDTSKYGGEDSPTVAYSIL | |
| | VIAKVEKGKAKKLKTVKELVGITIMEQSAFEKDPVKFLEDKGYQDIQEHLII | |
| | KLPKYSLFELENGRKRLLASAGELQKANELALPQKLVILLYHAKNIESSSEK | |
| | SEDESHHRYYVSNHYKEFDEIFDQIVEFSERYILADKNIEKIRELFDQNESL | |
| | SISELAQSFINLFTFTALGAPADFKFLNKDIDRKRYTSPSEILNSTLIHQSI | |
| | TGLYETRIDLSKLGGD | |
| CasEnd-245 | MKKPYSIGLDIGTNSVGWAVVTDDYKVPAKKMKVLGNTDKSHIKKNLLGALL | 205 |
| | FDSGNTAEDRRLKRTARRRYTRRRNRILYLQEIFSEEMGKVDDSFFHRLEDS | |
| | FLVPEDKRGERHPIFGNLEEEVKYHENFPTIYHLRKYLADNPEKADLRLVYL | |
| | ALAHIIKFRGHFLIEGKFDTRNNDVQRLFQEFLAVYDNTFENSSLQEQNVQV | |
| | EEILTDKISKSAKKDRVLKLFPNEKSNGRFAEFLKLIVGNQADFKKHFELEE | |
| | KAPLQFSKDTYEEDLEVLLAQIGDEYAELFLSAKKLYDSILLSGILTVTDVS | |
| | TKAPLSASMIQRYNEHQMDLTQLKQFIRQKLSDKYNEVESDVSKNGYAGYID | |
| | GKTNQEDFYKYLKKLLNKIEGSGYFLDKIEREDFLRKQRTFDNGSIPHQIHL | |
| | QEMRAIIRRQAEFYPFLAENQDKIEKILTFRIPYYVGPLARGKSDFAWLSRK | |
| | SADKITPWNFDEIVDKESSAEAFINRMTNYDLYLPNQKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASYGTYHDLRKILKDKDFLDNSKNEKILEDIVLTLT | |
| | LFEDREMIRKRLENYSDLLTKEQLKKLERRHYTGWGRLSAKLIHGIRNKESR | |
| | KTILDYLIDDGNSNRNFMQLINDDALSFKEEIAKAQVIGETDNLNQVVSDIA | |
| | GSPAIKKGILQSLKIVDELVKIMGTHQPENIVVEMARENQFTNQGRRNSQQR | |
| | LKGLTDSIKEFGSQILKEHPVENSQLQNDRLFLYYLQNGRDMYTGEELDIDY | |
| | LSQYDIDHIIPQAFIKDNSIDNRVLVSSKEARGKSDDVPSKEVVRKMKSYWS | |
| | KLLSAKLITQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLGKYPQLEPEFVYGEYPKFNSHKLVAKSKSEENKATAKKFFYS | |
| | NIMNFFKKDVKLADGSIIERPMIERNDETGEIIWDKDKHISTVKKVLSYPQV | |
| | NIVKKVEEQTGGFSKESILPKGNSDKLIPRKTKWDTKKYGGFDSPIVAYSVL | |
| | VIADIEKGKSKKLKTVKALVGITIMEKMTFERNPVAFLERKGYRNIQEENII | |
| | KLPKYSLFELENGRKRLLASARELQKGNEIVLPNHLGTLLYHAKNIHKVDEK | |
| | EEDIPKHLDYVDKHKDEFKELLDVVSNFSKKYTLAEKNLEKIKELYAQNNGA | |
| | DIKELASSFINLLTFTALGAPATFKFFDKNIDRKRYTSTTEILNATLIHQSI | |
| | TGLYETRIDLSKLGGD | |
| CasEnd-246 | MKKKYSIGLDIGTNSVGWAVITDEYKVPSKKFKVLGNTDRQSIKKNLLGALL | 206 |
| | FDSGETAEATRLKRTARRRYTRRKNRILYLQEIFSEEMAKVDDSFFHRLEES | |
| | FLVEEDKRGERHPIFGNIVDEVAYHEKYPTIYHLRKKLADSTEKADLRLIYL | |
| | ALAHIIKFRGHFLIEGDLNSENSDVQKLFIQLVQTYNQLFEENHINEEGVDA | |
| | SAILSARLSKSRRLENLIAQFPGEKKNGLFGNLIALSLGLTPNFKSNEDLAE | |
| | DAKLQLSKDTYDEDLDNLLAQIGDQYADLFSAAKNLSDAILLSGILTVNDEK | |
| | TKAPLSASMIKRYDEHHQDLTLLKALVRQQLPEKYKEIFFDQSKNGYAGYID | |
| | GGASQEEFYKFIKPILEKMDGTEEFLDKINREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQSFIERMTNFDKNLPNEKVLPKHSLLYEYFTV | |
| | YNELTKVKYVTEGMGKPEFLSAEQKEEIVDLLFKKNRKVTVKKLKEDLFKKI | |
| | ECFDSVEISGVEDRFNASLGTYHDLLKIIKDKDFLDNEENEDILEDIVLTLT | |
| | LFEDREMIEERLEKYADLFDKKVLKQLKRRRYTGWGRLSAKLINGIRDKQSG | |
| | KTILDFLKDDGFANRNFMQLIHDDSLTFKEEIQKAQVSGQTDSLHEVIANLA | |
| | GSPAIKKGILQSIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTETDENDKLIREVKIITLKSKLVSDFRKDFGFYKVREINHYHHAHDAY | |
| | LNAVVGTALIKKYPKLESEFVYGDYKVYDVRKMIAKSEQEIGKATAKYFFYS | |
| | NIMNFFKSEITLANGEIRKRPQIETNEETGEIVWDKEKDFATVKKVLSMPQV | |
| | NIVKKTEVQTGGFSKESILPKRNSDKLIARKKDWDTKKYGGFDSPTVAYSVL | |
| | VVAKVEKGKSKKLKPVKELVGITIMERSSFEKDPIAFLESKGYKDIQKDKII | |
| | KLPKYSLFELENGRKRLLASAGELQKGNELALPNKYVILLYHASHYEKLKES | |
| | EEDNKEHQEYVEQHRDYFDEIFEQISEFSERYILADKNLEKIEELYKENEDK | |
| | DISELAENFIHLFTFTALGAPAAFKFFDATIDRKRYTSTTEILNATLIHQSI | |
| | TGLYETRIDLSQLGGD | |
| CasEnd-247 | MDKPYSIGLDIGTNSVGWAVVTDEYKVPSKKFKVLGNTDRHSIKKNLLGALL | 207 |
| | FDSGETAEATRLKRTARRRYTRRKNRILYLQEIFAEEMNKVDPSFFHRLEES | |
| | FLVEEDKRGERHPIFGNIVEEVAYHEKYPTIYHLRKHLADSTEKADLRLIYL | |
| | ALAHIIKFRGHFLIEGDLNAENTDVQELFKDFVQVYDQTFEESHLSEETVDA | |
| | EEILTEKISKSRKLENLIKQFPNEKKNGLFGNLLALSLGLQPNFKSNFKLSE | |
| | DAKLQFSKDTYDEDLENLLGQIGDEYADLFVAAKNLYDAILLSGILTVTTEI | |
| | TKAPLSASMIKRYDEHHQDLTLLKKFIRQNLPEKYKEIFFDKSKNGYAGYID | |
| | GGASQEDFYKYIKNILSKLDGSEYFLDKIEREDFLRKQRTFDNGSIPHQIHL | |
| | QELKAILRRQGEYYPFLKENQEKIEKILTFRIPYYVGPLARGNSRFAWASRK | |
| | SDETITPWNFEEVVDKEASAQAFITRMTNFDKNLPEEKVLPKHSLLYEKFTV | |
| | YNELTKVKYVTEGMRKPAFFDANQKQEIFDHLFKKNRKVTKKKLLEFLFKEF | |
| | DEFRIVDISGVEKSFNASLGTYHDLLKIIKDKEFLDNEENEKILEDIVLTLT | |
| | LFEDREMIKKRLEKYADLFTKKVLKKLERRRYTGWGRLSAKLINGIRDKQSG | |
| | KTILDYLIDDGFANRNFMQLIHDDSLSFKEEIAKAQVIGETDSLHQVIANLA | |
| | GSPAIKKGILQSIKIVDELVKVMGRYNPENIVVEMARENQTTQKGQRNSRER | |
| | LKRLEEAIKELGSKILKEHPVENTQLQNDKLYLYYLQNGKDMYTGEELDIDR | |
| | LSDYDVDHIIPQSFIKDDSIDNRVLTSSAKARGKSDDVPSEEVVKKMKSFWQ | |
| | QLLKAKLISQRKFDNLTKAERGGLTELDKAGFIKRQLVETRQITKHVAQILD | |
| | SRFNTKYDENDKLIRKVKIITLKSKLVSNFRKDFELYKVREINDYHHAHDAY | |
| | LNAVVGTALIKKYPKLESEFVYGDYPVYNSYKMIAKSEQEIGKATAKYFFYS | |
| | NIMNFFKSDITLANGEIRKRPLIETNEETGEIAWDKDKDIATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKNPIAFLEDKGYQNIQKENII | |
| | KLPKYSLFELENGRKRLLASAGELQKGNELALPNKYVTLLYHAKHYEKLKES | |
| | PEDNPEHLEYVDKHRDEFDEILDQISEFSKRYILADKNLEKIKELYKKNEDA | |
| | DIEELASSFINLLTFTALGAPAAFKFFGATIDRKRYTSTTEILNATLIHQSI | |
| | TGLYETRIDLSQLGGD | |
| CasEnd-248 | MKKKYSIGLDIGTNSVGWAVITDEYKVPSKKFKVLGNTDRQSIKKNLIGALL | 208 |
| | FDSGETAEATRLKRTARRRYTRRKNRIRYLQEIFAEEMSKVDDSFFHRLEES | |
| | FLVEEDKRDERHPIFGNIVDEVAYHEKYPTIYHLRKKLADSTEKADLRLVYL | |
| | ALAHIIKFRGHFLIEGDLNSENSDVDKLFIQLVQTYNQLFEENPINEEGVDA | |
| | KAILSAKLSKSRRLENLIAQIPGEKKNGLFGNLIALSLGLTPNFKSNEDLAE | |
| | DAKLQLSKDTYDEDLDNLLAQIGDQYADLFLAAKNLSDAILLSDILTVDSES | |
| | TKAPLSASMIKRYDEHHQDLTLLKALVREQLPEKYKEIFFDKSKNGYAGYID | |
| | GGASQEDFYKYIKPILEKLDGAEELLEKINREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPDEKVLPKHSLLYEYFTV | |
| | YNELTKVKYVTEGMRKPEFLSAEQKEEIVDLLFKKNRKVTVKQLKENYFKKI | |
| | ECFDSVEISGVEDRFNASLGTYHDLLKIIKDKEFLDNEENEDILEDIVLTLT | |
| | LFEDKEMIEERLKKYANLFDKKVLKQLKRRRYTGWGRLSKKLINGIRDKQSG | |
| | KTILDFLKSDGFANRNFMQLIHDDSLTFKEEIQKAQVKGQGDSLHEQIANLA | |
| | GSPAIKKGILQTIKIVDEIVKVMGRHKPENIVIEMARENQTTQKGQKNSRER | |
| | LKRLEEVLKELGSQILKEHPVENTQLQNDKLYLYYLQNGKDMYTGDELDIDR | |
| | LSDYDVDHIIPQSFIKDDSIDNKVLTSSKEARGKSDNVPSEEVVKKMKSYWR | |
| | QLLNAKLISQRKFDNLTKAERGGLSELDKAGFIKRQLVETRQITKHVAQILD | |
| | SRFNTEYDENDKLIRRVKIITLKSKLVSDFRKDFGFYKVREINDYHHAHDAY | |
| | LNAVVGTALIKKYPKLESEFVYGDYKVYDSRKMIAKSEQEIGKATAKMFFYS | |
| | NIMNFFKSEIKLADGEIRKRPQIETNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKKKKLKSVKELVGITIMERSAFEKDPVDFLENKGYKDIQKDLII | |
| | KLPKYSLFELENGRKRLLASAGELQKGNELALPNKYVTFLYLASHYEKLKGS | |
| | PEDEEQHQEYVEQHKYYFDEILEQIEEFSERYILADKNLEKILSLYNEKSDK | |
| | SISEQAENISNLFTFTALGAPAAFKFFDTTIDRKRYTSTKEVLNATLIHQSI | |
| | TGLYETRIDLSQLGGD | |
| CasEnd-249 | MDKKYSIGLDIGTNSVGWAVVTDEYKVPSKKFKVLGNTDRKSIKKNLLGALL | 209 |
| | FDSGETAEATRLKRTARRRYTRRKNRIRYLQEIFAEEMAKVDDSFFHRLEES | |
| | FLVEEDKRGERHPIFGNIVDEVAYHEKYPTIYHLRKHLADSDEKADLRLVYL | |
| | ALAHIIKFRGHFLIEGDLNSENSDVQKLFKELVQVYDQTFEESHLEEEGVDA | |
| | EAILTEKLSKSRRLENLIAQFPGEKKNGLFGNLIALSLGLTPNFKSNEDLSE | |
| | DAKLQFSKDTYDEDLEELLGQIGDEYADLFVAAKNLYDAILLSGILTVDDES | |
| | TKAPLSASMIKRYDEHHQDLTLLKAFIREQLPEKYKEIFFDQSKNGYAGYID | |
| | GGASQEEFYKYLKKILEKIDGSEEFLDKIEREDFLRKQRTFDNGSIPHQIHL | |
| | QELHAIIRRQEKYYPFLKENQEKIEKILTFRIPYYVGPLARGNSRFAWLSRK | |
| | SDETITPWNFDEVVDKEKSAEAFIERMTNFDKNLPNEKVLPKHSLLYEKFTV | |
| | YNELTKVKYVTEGMRKPEFFDAEQKQEIVDGVFKKNRKVTKKQLKEYLFKEF | |
| | DEFRIVEISGVEDRFNASLGTYHDLLKIIKDKDFLDNEENEEILEDIILTLT | |
| | LFEDREMIKKRLEKYADLFDKKQLKKLERRRYTGWGRLSAKLINGIRDKQSG | |
| | KTILDYLIDDGFANRNFMQLIHDDSLTFKEEIEKAQVIGDSESLHELIANLA | |
| | GSPAIKKGILQSLKIVDELVKVMGRYEPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIRDVKIITLKSKLVSDFRKDFELYKVREINNYHHAHDAY | |
| | LNAVVGTALIKKYPKLESEFVYGDYKVYDVRKMIAKSEQEIGKATAKYFFYS | |
| | NIMNFFKSDITLANGEIRKRPLIETNEETGEIVWDKEKDIATVRKVLSMPQV | |
| | NIVKKTEVQTGGFSKESILPKRDSDKLIARKKDWDTKKYGGFDSPTVAYSVL | |
| | VIADVEKGKAKKLKTVKELVGITIMERSAFEKNPVAFLEDKGYQNIKKDNII | |
| | KLPKYSLFELENGRKRLLASAGELQKGNELALPQKYVTLLYHAKHYEKLKGS | |
| | PEDNEEKQLYVEEHKDEFDEILDQISEFAKRYILADANLEKLKKLYEKNRDA | |
| | SIEELAENFIHLLTFTALGAPAAFKFFGKTIDRKRYTSTKEILNATLIHQSI | |
| | TGLYETRIDLSKLGGD | |
| CasEnd-250 | MDKKYSIGLDIGTNSVGWAVITDEYKVPSKKFKVLGNTGRKSIKKNLIGALL | 210 |
| | FDSGETAEATRLKRTARRRYTRRKNRIRYLQEIFADEMSKVDDSFFHRLEES | |
| | FLVEEDKKNERHPIFGNIVEEVAYHEKYPTIYHLRKKLVDSDEKADLRLIYL | |
| | ALAHMIKFRGHFLIEGDLNPDNSDVDKLFHQLVQTYNQLFEEDPIEAEGVDA | |
| | KAILSARLSKSRRLENLIAELPGEKKNGLFGNLIALSLGLTPNFKSNFDLAE | |
| | DAKLQLSKDTYDDDLENLLGQIGDQYADLFVAAKNLSDAILLSDILRVNTES | |
| | TKAPLSASMIKRYDEHHQDLTLLKQLVRKQLPEKYKEIFFDQSKNGYAGYID | |
| | GGASQEEFYKFIKPILEKMDGTEELLAKLEREDLLRKQRTFDNGSIPHQIHL | |
| | GELHAILRRQEKYYPFLKENREKIEKILTFRIPYYVGPLARGNSRFAWMTRK | |
| | SDETITPWNFEEVVDKGASAQSFIERMTNFDKNLPEEKVLPKHSLLYEYFTV | |
| | YNELTKVKYVTEGMRKPEFLSGEQKKAIVDLLFKKNRKVTVKQLKEYYFKNF | |
| | ECFDSVEISGVEDRFNASLGTYHDLLKIIKDKDELDNEENEKILEDIVLTLT | |
| | LFEDREMIKKRLEKYADLFDKKVMKQLKRRRYTGWGRLSRKLINGIRDKQSG | |
| | KTILDFLKEDGFTNRNFMQLIHDDSLTFKDDIKKAQVIGQSDSLHEHIANLA | |
| | GSPAIKKGILQTVKVVDELVKVMGRYKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNGETGEIVWDKERDFATVRKVLSMPQV | |
| | NIVKKTEVQTGGFSKESILPKRDSDKLIARKKDWDPKKYGGFDSPTVAYSVL | |
| | VVAKVEKGKAKKLKTVKELLGITIMERSEFEKDPIAFLEDKGYKEVKKDLII | |
| | KLPKYSLFELENGRKRMLASAGELQKGNELALPSKYVNFLYLASNYEKLKGS | |
| | EEDNKKKQLYVEQHKEYLDEIIDQISEFSERVILADANLEKVLSAYEKHRDK | |
| | SIEEQAENIIHLFTLTNLGAPAAFKYFNTNIDRKRYTSTKEVLNATLIHQSI | |
| | TGLYETRIDLSQLGGD | |
| CasEnd-251 | MKKKYSIGLDIGTNSVGWAVITDEYKVPSKKFKVLGNTDRHSIKKNLIGALL | 211 |
| | FDSGETAEATRLKRTARRRYTRRKNRIRYLQEIFASEMAKVDDSFFHRLEES | |
| | FLVEEDKDHERHPIFGNIVDEVAYHEKYPTIYHLRKKLADSDEKADLRLIYL | |
| | ALAHMIKFRGHFLIEGDLNSENTDVDKLFIQLVQTYNQLFEENPINEEGVDA | |
| | KAILSAKLSKSRRLENLIAQFPGEKRNGLFGNLIALSLGLTPNFKSNFDLAE | |
| | DAKLQLSKDTYDEDLDNLLGQIGDQYADLFLAAKNLSDAILLSDILTVNDET | |
| | TKAPLSASMIKRYDEHHQDLTLLKALVRQQLPEKYKEIFFDDTKNGYAGYID | |
| | GGASQEEFYKYIKPILEKLDGTEYFLAKINREDFLRKQRTFDNGSIPHQIHL | |
| | GELHAILRRQEEFYPFLKENREKIEKILTFRIPYYVGPLARGNSRFAWMKRK | |
| | SDETITPWNFEEVVDKEASAQAFIERMTNFDKNLPNEKVLPKHSLLYEYFTV | |
| | YNELTKVKYVTEGMRKPEFLSAEQKEAIVDLLFKTNRKVTVKQLKEDYFKKI | |
| | DCFDSVEISGVEDRFNASLGTYHDLLKIIKDKDELDNEENEDILEDIVLTLT | |
| | LFEDREMIEERLEKYADLFDKKVLKQLKRRRYTGWGRLSAKLINGIRDKQSG | |
| | KTILDFLKSDGFANRNFMQLIHDDSLTFKEDIQKAQVKGQSDSLHEQIADLA | |
| | GSPAIKKGILQSIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTNRDENDKLIRDVKIITLKSKLVSDFRKDFEFYKVREINDYHHAHDAY | |
| | LNAVVGTALIKKYPKLESEFVYGDYKVYDVRKMIAKSEQEIGKATAKYFFYS | |
| | NIMNFFKTDITLANGEIRKRPLIETNEETGEIVWDKTKDFATVRKVLSMPQV | |
| | NIVKKTEVQTGGFSKESILPKRESDKLIARKKDWDPKKYGGFDSPTVAYSVL | |
| | VVAKVEKGKAKKLKTVKELVGITIMERSSFEKNPVAFLEDKGYKDIQEELII | |
| | KLPKYSLFELENGRKRLLASAGELQKGNELALPSKYVTFLYLASHYEKLKGK | |
| | PEDNEQKKEYVKQHKDEFDEILDQISEFSERYILADANLDKVLSLYNNNRDK | |
| | DISELAENFIHLFTFTALGAPAAFKFFDTDIDRKRYTSTTEVLNATLIHQSI | |
| | TGLYETRIDLSQLGGD | |
| CasEnd-252 | MKKPYSIGLDIGTNSVGWAVVTDEYKVPSKKFKVLGNTDRSSIKKNLLGALL | 212 |
| | FDSGETAEATRLKRTARRRYTRRKNRILYLQEIFAEEMNKVDDSFFHRLEES | |
| | FLVEEDKRGERHPIFGNIVDEVAYHEKYPTIYHLRKHLADSTEKADLRLVYL | |
| | ALAHIIKFRGHFLIEGDLDAENTDVQKLFKKFVEVYDQTFEESHLSEETVDA | |
| | EAILTEKLSKSRRLENLIAQFPNEKKNGLFGNLIALSLGLQPNFKINFELSE | |
| | DAKLQFSKDTYDEDLENLLGQIGDEYADLFVAAKNLYDAILLSGILTVNDES | |
| | TKAPLSASMIKRYDEHHQDLTLLKAFIRKNLPEKYKEIFFDESKNGYAGYID | |
| | GGASQEEFYKYIKKLLSKIDGSEYFLDKIEREDFLRKQRTFDNGSIPHQIHL | |
| | QELKAILRRQEEYYPFLKENQEKIEKILTFRIPYYVGPLARGNSRFAWASRK | |
| | SDETITPWNFDEVVDKEKSAEAFIERMTNNDKNLPDEKVLPKHSLLYEKFTV | |
| | YNELTKVKYVTEGMGKAQFFDANMKQEIFDGLFKKERKVTKKKLLDFLFKEF | |
| | DEFRIVDISGVEDAFNASLGTYHDLLKIIKDKEFLDNEENEKILEDIVLTLT | |
| | LFEDREMIKKRLSKYADLFDKKQLKKLERRRYTGWGRLSAKLINGIRDKQTG | |
| | KTILDYLIDDGFANRNFMQLIHDDSLSFKEEIAKAQVIGDSDSLHEVIADLA | |
| | GSPAIKKGILQSLKIVDELVKVMGRYAPENIVVEMARENQTTQKGQRNSRER | |
| | LKRLEEAIKELGSKILKEHPVENTQLQNDKLYLYYLQNGKDMYTGEELDIDR | |
| | LSDYDVDHIIPQSFIKDDSIDNRVLVSSAKARGKSDDVPSEEVVKKMKSFWS | |
| | QLLNAKLISQRKFDNLTKAERGGLTELDKAGFIKRQLVETRQITKHVAQILD | |
| | ERFNTEFDENNKLIRNVKIITLKSKLVSQFRKEFELYKVREINDYHHAHDAY | |
| | LNAVVGKALIKKYPKLESEFVYGDYPVYNSYKLVAKSDSEIGKATAKMFFYS | |
| | NIMNFFKSDIKLADGTIVERPQIEVNEETGEIVWDKEKHIATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKNPVAFLEDKGYQNIQEENII | |
| | KLPKYSLFELENGRKRLLASAGELQKGNELALPQKYVTLLYHAKHYEKLKEK | |
| | SEDREKHLEYVEQHRDEFDEILDQISEFSKRYILADKNLEKIEELYNKNEDA | |
| | SIEELASSFINLLTFTALGAPAAFKFFGKTIDRKRYTSTKEILNATLIHQSI | |
| | TGLYETRIDLSKLGGD | |
| CasEnd-253 | MDKKYSIGLDIGTNSVGWAVITDEYKVPSKKFKVLGNTDRHSIKKNLIGALL | 213 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTDKADLRLIYL | |
| | ALAHMIKFRGHFLIEGDLNSDNSDVDKLFIQLVQTYNQLFEENPINESGVDA | |
| | KAILSAKLSKSRRLENLIAQLPGEKKNGLFGNLIALSLGLTPNFKSNEDLSE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDKSKNGYAGYID | |
| | GGASQEEFYKYIKPILEKMDGTEELLAKINREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQAFIERMTNFDKNLPNEKVLPKHSLLYEYETV | |
| | YNELTKVKYITEGMRKPEFLSGEQKKAIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEERLKKYAHLFDKKVLKQLKRRRYTGWGRLSRKLINGIRDKQSG | |
| | KTILDFLKSDGFANRNFMQLIHDDSLTFKEAIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKTKKLKTVKELVGITIMERSSFEKDPVAFLEAKGYKEVRKDLII | |
| | KLPKYSLFELENGRKRLLASAGELQKGNELALPQKYVTFLYLASHYEKLKGK | |
| | PEDNEQKLEYVEQHRDEFDEIFEQISEFSERYILADKNLDKILSLYNNIEDK | |
| | SIEELAENFIHLFTFTSLGAPAAFKFFDTTIDRKRYTSTTEILDATLIHQSI | |
| | TGLYETRIDLSQLGGD | |
| CasEnd-254 | MDKPYSIGLDIGTNSVGWAVVTDEYKVPSKKFKVLGNTDRHSIKKNLLGALL | 214 |
| | FDSGETAEATRLKRTARRRYTRRKNRIRYLQEIFAEEMNKVDDSFFHRLEES | |
| | FLVEEDKRGERHPIFGNIVEEVAYHENYPTIYHLRKELADSTEKADLRLVYL | |
| | ALAHIIKFRGHFLIEGDLNSENTDVQKLFIKFVQTYNNTFEESHLSEINVDA | |
| | ESILTAKLSKSRRLENLIKYFPNEKKNGLFGNLIALSLGLQPNFKTNEDLSE | |
| | DAKLQFSKDTYEEDLENLLAQIGDQYADLFVAAKNLYDAILLSGILTVNTEI | |
| | TKAPLSASMIKRYDEHHQDLTLLKDFIRQQLPEKYKEIFFDKSKNGYAGYID | |
| | GGAKQEEFYKYIKPILEKIDGTEYFLDKINREDFLRKQRTFDNGSIPHQIHL | |
| | KELHAIIRRQAEFYPFLKENQEKIEKILTFRIPYYVGPLARGNSRFAWMSRK | |
| | SDETITPWNFEEVVDKEASAQAFIERMTNYDKNLPEEKVLPKHSLLYEKFTV | |
| | YNELTKVKYVTERMRKPAFFDANQKQEIVDGLFKKNRKVTVKQLKEFLFKEF | |
| | DEFDSVEISGVEKRFNASLGTYHDLLKIIKDKDFLDNEENEKILEDIVLTLT | |
| | LFEDREMIRKRLSKYADLFDKKVLKKLKRRRYTGWGRLSAKLINGIRDKQTG | |
| | KTILDYLIDDGFANRNFMQLIHDDSLTFKEEIAKAQVIGKTDSLHEVIADLA | |
| | GSPAIKKGILQSLKIVDELVKVMGRHNPENIVIEMARENQTTQKGQRNSRER | |
| | LKRLEESIKELGSNILKEHPVDNTQLQNDKLYLYYLQNGKDMYTGEELDINR | |
| | LSDYDVDHIIPQSFIKDDSIDNKVLTSSAKARGKSDNVPSIEVVRKMKSYWE | |
| | QLLNAKLISQRKFDNLTKAERGGLTELDKAGFIKRQLVETRQITKHVAQILD | |
| | SRFNTKYDENDKLIRDVKIITLKSKLVSNFRKDFGLYKVREINDYHHAHDAY | |
| | LNAVVGTALIKKYPKLESEFVYGDYPVYNSYKMIAKSEQEIGKATAKYFFYS | |
| | NIMNFFKSDITLANGEIRKRPLIETNDETGEIVWDKKKDFATVRKVLSMPQV | |
| | NIVKKTEVQTGGFSKESILPKRNSDKLIARKKDWDTKKYGGFDSPTVAYSVL | |
| | VIAKIEKGKAKKLKTVKELVGITIMERSSFEKNPVAFLEKKGYQNIQKEVII | |
| | KLPKYSLFELENGRKRLLASAGELQKGNELALPSKYVTLLYHASRYEKLKES | |
| | PEDNEKHLEYVEKHREEFDEILDQISEFSKRYILADKNLEKILELYDKNNEA | |
| | SIEELAESFINLLTFTALGAPAAFKFFGTTIDRKRYTSTTEILSATLIHQSI | |
| | TGLYETRIDLSQLGGD | |
| CasEnd-255 | MKKKYSIGLDIGTNSVGWAVITDEYKVPSKKFKVLGNTDRKSIKKNLLGALL | 215 |
| | FDSGETAEATRLKRTARRRYTRRKNRIRYLQEIFAEEMSKVDDSFFHRLEES | |
| | FLVEEDKKHERHPIFGNIVDEVAYHEKYPTIYHLRKKLADSTEKADLRLIYL | |
| | ALAHMIKFRGHFLIEGDLNPENSDVQKLFIQLVQTYNQLFEESPLEESTVDA | |
| | KAILSAKLSKSRRLENLIAQFPGEKKNGLFGNLIALSLGLTPNFKSNEDLAE | |
| | DAKLQLSKDTYDEDLDNLLAQIGDQYADLFLAAKNLSDAILLSDILTVNDEI | |
| | TKAPLSASMVKRYDEHHQDLTLLKALVRKQLPEKYKEIFFDKSKNGYAGYID | |
| | GGASQEEFYKFIKPILEKVDGTEELLEKLNREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAEAFIERMTNFDKNLPEEKVLPKHSLLYEMFTV | |
| | YNELTKVKYVTEGMGKPEFLSGEQKQEIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLAKYADLFDKKVLKQLKRRRYTGWGRLSRKLINGIRDKQSG | |
| | KTILDFLKDDGFANRNFMQLIHDDSLTFKEEIQKAQVSGKGDSLHEVIANLA | |
| | GSPAIKKGILQSVKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIREVKIITLKSKLVSDFRKDFQFYKVREINDYHHAHDAY | |
| | LNAVVGTALIKKYPKLESEFVYGDYKVYDVRKMIAKSDPEIGKATAKYFFYS | |
| | NIMNFFKTEITLANGEIFKRPVIETNKETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKKKKLKTVKELVGITIMERSSFEKDPIAFLETKGYKDVQEDLII | |
| | KLPKYSLFELENGRKRLLASAGELQKGNELALPQKYVTFLYLAKRYEKLKGS | |
| | LEDNEGKQEYVEQHKHYFDEIMDQIKEFSERYILADKNLEKLLSLFAENRDK | |
| | DIEELAENFIHLFTLTSLGAPAAFKFFDTTIDRKRYTSTSEVLNATLIHQSI | |
| | TGLYETRIDLSQLGGD | |
| CasEnd-256 | MDKKYSIGLDIGTNSVGWAVITDEYKVPSKKFKVLGNTDRHSIKKNLIGALL | 216 |
| | FDSGETAEATRLKRTARRRYTRRKNRICYLQEIFSNEMAKVDDSFFHRLEES | |
| | FLVEEDKRGERHPIFGNIVDEVAYHEKYPTIYHLRKHLADSTEKADLRLVYL | |
| | ALAHIIKFRGHFLIEGDLNSENTDVQKLFKQFVTTYDQTFEESHLNEETVDA | |
| | KSILTEKLSKSRRLENLIKLFPGEKKNGLFGNLIALSLGLTPNFKSNEDLSE | |
| | DAKLQFSKDTYDEDLENLLGQIGDEYADLFAAAKNLYDAILLSGILTVNDES | |
| | TKAPLSASMIKRYDEHHQDLTLLKTFVRQQLPEKYKEIFFDESKNGYAGYID | |
| | GGASQEEFYKYIKPILEKMDGSEYFLDKIEREDFLRKQRTFDNGSIPHQIHL | |
| | KELHAILRRQEEFYPFLKENREKIEKILTFRIPYYVGPLARGNSRFAWASRK | |
| | SDETITPWNFDEVVDKEASAEAFIERMTNNDKNLPDEKVLPKHSLLYEKFTV | |
| | YNELTKVKYVTEGMGKAQFFDAEQKQEIVDLLFKKYRKVTKKKLLDFLDKEF | |
| | EEFRIVDISGVEDAFNASLGTYHDLLKIIKDKDFLDNEENEDILEDIVLTLT | |
| | LFEDREMIEKRLEKYADLFDKKVLKKLERRRYTGWGRLSKKLINGIRDKQTG | |
| | KTILDYLISDGFANRNFMQLIHDDSLSFKEEIAKAQVIGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKNPIAFLEDKGYQNIQEEKII | |
| | KLPKYSLFELENGRKRLLASAGELQKGNELALPAKYVTLLYHAKHYEKLKEK | |
| | PEDNEKHLEYVTKHRDEFKEILDQISEFSERYILADKNLSKIKELYSKNESY | |
| | SIEELASSFINLLTFTALGAPAAFKFLGKTIDRKRYTSTTEILNATLIHQSI | |
| | TGLYETRIDLSKLGGD | |
| CasEnd-257 | MKKPYSIGLDIGTNSVGWAVVTDDYKVPAKKMKVLGNTDKSHIKKNLLGALL | 217 |
| | FDSGNTAEDRRLKRTARRRYTRRRNRILYLQEIFSEEMNKVDDSFFHRLEDS | |
| | FLVPEDKRGERHPIFGNLEEEVKYHENFPTIYHLRKYLADSPEKADLRLVYL | |
| | ALAHIIKFRGHFLIEGKLDTRNNDVQRLFQEFLAVYDNTFENSSLQEQNVQV | |
| | EEILTDKISKSAKKDRVLKLFPNEKSNGRFAEFLKLIVGNQADFKKHFELEE | |
| | KAPLQFSKDTYEEDLENLLAQIGDDYAELFLSAKKLYDSILLSGILTVTDVS | |
| | TKAPLSASMIKRYNEHQSDLTQLKQFIRQKLSDKYNEVFSDVSKDGYAGYID | |
| | GKTNQEAFYKYLKGLLNKIEGSGYFLDKIEREDFLRKQRTFDNGSIPHQIHL | |
| | QEMRAIIRRQAEFYPFLAENQDKIEKILTFRIPYYVGPLARGKSDFAWLSRK | |
| | SADKITPWNFDEIVDKESSAEAFINRMTNYDLYLPNEKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASYGTYHDLRKILKDKDFLDNSKNEKILEDIVLTLT | |
| | LFEDREMIRKRLENYSDLLTKEQLKKLERRHYTGWGRLSAKLINGIRNKESR | |
| | KTILDYLIDDGNSNRNFMQLINDDALSFKEEIAKAQVIGETDNLNQVVSDIA | |
| | GSPAIKKGILQSLKIVDELVKIMGGHQPENIVVEMARENQFTNQGRRNSQQR | |
| | LKGLTDSIKEFGSQILKEHPVDNSQLQNDRLFLYYLQNGRDMYTGEELDIDY | |
| | LSQYDIDHIIPQAFIKDNSIDNRVLVSSKEARGKSDDVPSKDVVRKMKSEWS | |
| | KLLSAKLITQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLKKYPKLEPEFVYGDYPKFNGYKFVSQIKEEENKATAKKFFYS | |
| | NIMNFFKSDIKLADGQIVERPMIERNDETGEIIWDKTKHISTVKKVLSYPQV | |
| | NIVKKVEEQTGGFSKESILPKGNSDKLIPRKTKWDTKKYGGFDSPIVAYSVL | |
| | VIADIEKGKSKKLKTVKALVGITIMEKMTFEKNPVAFLERKGYQNIQEENII | |
| | KLPKYSLFELENGRKRLLASARELQKGNELVLPNHLVILLYHAKNIHKIDEK | |
| | PEDIPKHLEYVEKHRDEFKELLDVVSNFSKKYTLAEGNLEKIKELYAQNNSA | |
| | DIKELASSFINLLTFTALGAPAAFKFFDKNIDRKRYTSTTEILNATLIHQSI | |
| | TGLYETRIDLSKLGGD | |
| CasEnd-258 | MKKPYSIGLDIGTNSVGWAVITDEYKVPSKKFKVLGNTDRHSIKKNLLGALL | 218 |
| | FDSGETAEATRLKRTARRRYTRRKNRIRYLQEIFSSEMAKVDDSFFHRLEES | |
| | FLVEEDKRHERHPIFGNIVDEVAYHEKYPTIYHLRKKLADSTQKADLRLIYL | |
| | ALAHMIKFRGHFLIEGDLNSDNSDVDKLFIQLVQTYNQLFEENPINESGVDA | |
| | KAILSEKLSKSRRLENLIAQLPGEKKNGLFGNLIALSLGLTPNFKSNFKLAE | |
| | DAKLQFSKDTYDEDLDNLLAQIGDQYADLFLAAKNLSDAILLSGILTVNDEI | |
| | TKAPLSASMIKRYDEHHQDLTLLKALVRQQLPEKYKEIFFDQTKNGYAGYID | |
| | GGASQEEFYKFIKPILEKMDGTEELLTKLNREDLLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNEDKNLPNEKVLPKHSLLYEYFTV | |
| | YNELTKVKYVTEGMRKPEFLSGKQKQEIVDLLFKKNRKVTVKQLKDDYFKKI | |
| | DCFDSVEISGVEDSFNASLGTYHDLLKIIKDKDELDNEENEDILEDIVLTLT | |
| | LFEDREMIEHRLSKYAHLFDKKVLKQLKRRRYTGWGRLSRKLINGIRDKQSG | |
| | KTILDFLKSDGFANRNFMQLIHDDSLTFKEAIQKAQVSGKTDSLHEQIANLA | |
| | GSPAIKKGILQTVKIVDELVKVMGRHNPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTESDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VIAKVEKGKSKKLKSVKELVGITIMERSSFEKDPVAFLEKKGYKNIQDDLII | |
| | KLPKYSLFELENGRKRMLASAGELQKGNELALPQHYVTFLYHASHYEKLKGR | |
| | PEDNEKKLYYVEQHRDYFDEIFSQIEEFSERYILADANLSKVKSLYNNNRDS | |
| | SIREQAENFIHLLTFTSLGAPAAFKFFDTTIDRKRYTSTTEVLDATLIHQSI | |
| | TGLYETRIDLSKLGGD | |
| CasEnd-259 | MDKKYSIGLDIGTNSVGWAVVTDDYKVPSKKFKVLGNTDRKSIKKNLLGALL | 219 |
| | FDSGETAEATRLKRTARRRYTRRKNRICYLQEIFSNEMAKVDDSFFHRLEES | |
| | FLVEEDKRGERHPIFGNIVEEVAYHEKFPTIYHLRKHLADSTEKADLRLVYL | |
| | ALAHIIKFRGHFLIEGDLSSENTDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVSDES | |
| | TKAPLSASMVKRYEEHHKDLTLLKDFIRKQLPEKYKEIFFDKSKNGYAGYID | |
| | GGASQEEFYKYLKKILEKIDGSEEFLDKIEREDFLRKQRTFDNGSIPHQIHL | |
| | QEMHAIIRRQEEYYPFLKENQEKIEKILTFRIPYYVGPLARGNSRFAWLSRK | |
| | SDETITPWNFDEVVDKEASAEAFIERMTNFDKNLPDEKVLPKHSLLYEKFTV | |
| | YNELTKVKYVTEGGKKPKFFDANLKQEIVDLLFKKERKVTKKQLLDFLVKEF | |
| | DEFRIVDISGVEDRFNASLGTYHDLLKILKDKDELDNEENEEILEDIVLTLT | |
| | LFEDREMIKQRLEKYADLFDKKQLKKLERRRYTGWGRLSAKLINGIRDKQTG | |
| | KTILDYLIDDGFANRNFMQLIHDDSLSFKEEIAKAQVIGQGDSLHETIANLA | |
| | GSPAIKKGILQSLKIVDELVKVMGRHEPENIVVEMARENQTTAKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLTDDDKAGFIKRQLVETRQITKHVAQILD | |
| | SRFNTEYDENGKLIRDVKIITLKSKLVSQFRKEFELYKVREINDYHHAHDAY | |
| | LNAVVGTALIKKYPKLESEFVYGDYPKYNLRKLIKKSDRERGKATAKMFFYS | |
| | NIMNFFKSDVKLADGDVRERPIIEVNEETGEIIWDKGKHIATVKKVLSMPQV | |
| | NIVKKTEVQTGGFSKESILPKRDSDKLIARKKDWDTKKYGGFDSPTVAYSVL | |
| | VVADVEKGKAKKLKTVKELVGITIMERSAFEKDPVAFLEDKGYQNIQKDNII | |
| | KLPKYSLFELENGRKRLLASAGELQKGNELALPNHLVTLLYHAKHIEKLDGK | |
| | PEDEKEKLLYVEKHRDEFDEIFDQISEFSKRYILADANLEKIKELYEKNFEA | |
| | SIEELASSFINLLTFTALGAPAAFKFFGKDIDRKRYTSTKEILNATLIHQSI | |
| | TGLYETRIDLSKLGGD | |
| CasEnd-260 | MDKKYSIGLDIGTNSVGWAVITDEYKVPSKKFKVLGNTDRQSIKKNLIGALL | 220 |
| | FDSGETAEATRLKRTARRRYTRRKNRICYLQEIFSEEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQTFEENPINASGVDA | |
| | KAILSERLSKSRRLENLIAQLPNEKKNGLFGNLIALSLGLTPNFKSNFDLAE | |
| | DAKLQLSKDTYDDDLDNLLGQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SDETITPWNFEEVVDKGASAQSFIERMTNFDKNLPNEKVLPKHSLLYEYFTV | |
| | YNELTKVKYVTEGMRKPAFLSGEQKKEIVDLLFKTNRKVTVKQLKEDYFKEI | |
| | ECFDSVEISGVEDRFNASLGTYHDLLKIIKDKDFLDNEENEDILEDIVLTLT | |
| | LFEDREMIEKRLKTYANLFDKKVMKQLKRRRYTGWGRLSRKLINGIRDKQSG | |
| | KTILDFLKSDGFANRNFMQLIHDDSLTFKEDIQKAQVSGQGDSLHEQIANLA | |
| | GSPAIKKGILQTIKVVDELVKVMGRHKPENIVIEMARENQTTQKGQKNSRER | |
| | MKRIEEGIKELGSQILKEHPVENTQLQNEKLYLYYLQNGRDMYTDQELDINR | |
| | LSDYDVDHIVPQSFLKDDSIDNKVLTSSKKARGKSDNVPSEEVVKKMKSYWR | |
| | QLLNAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKVYDSRKMIAKSEQEIGKATAKYFFYS | |
| | NIMNFFKSEITLANGTIRKRPVIETNEETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKTKKLKSVKELLGITIMERSSFEKNPVDFLEAKGYKEIQEDLII | |
| | KLPKYSLFELENGRKRLLASAGELQKGNELALPQKYVTFLYHASHYEKLKGK | |
| | PEDEEKKQLFVEQHNHYFDEIVEQIEEFSERYILADKNLEKIKSLYNNHEDY | |
| | SIREQAENIINLETLINLGAPAAFKYFDTTIDRKRYTSTKEILNATLIHQSI | |
| | TGLYETRIDLSQLGGD | |
| CasEnd-261 | MDKPYSIGLDIGTNSVGWAVVTDEYKVPSKKFKVLGNTDRKSIKKNLWGVLL | 221 |
| | FDSGETAEATRLKRTARRRYTRRKNRILYLQEIFAEEINKVDENFFHRLEES | |
| | FLVEEDKRGERHPIFANIVEEVAYHEDYPTIYHLRKHLADTPEKADLRLVYL | |
| | ALAHIIKFRGHFLIEGKEDVENTDIQETFKEFLEIYDETVEESELEIENIDV | |
| | ESILTDKISKSRRKEEVLKLFPNQKKNSIFAEFLKLIVGLTPNFKSFFNLEE | |
| | DAKLQFSKDTYEEDLEELLGQIGDEYAEVFVSAKRVYDSIVLSGILTVKDNS | |
| | TKAKLSASMVQRYDEHHQDLTKLKKFIRKNFPDEYKDIFFDQSKDGYAGYID | |
| | GGAKQEDFYKYLKKLLNKIEGSEYFLEKIENEDFLRKQRTFDNGSIPHQVHL | |
| | QEMKAIIKNQGEYYPFLKENQDKIQQILTFRIPYYVGPLARGNSRFAWLSRK | |
| | SDETITPWNFDDIIDKEKSAEKFIERMTNFDKNLPDEKVLPKHSLLYEKFTV | |
| | YNELTKVKYIDEQGKEKQFFDANLKQEIFNELFKKERKVTKKQLLDYLKKEF | |
| | YELRIVDISGVEDRFNASLSTYHDLKKILGNEEFLDDPKNAEMLEEIIKTLT | |
| | LFEDRKMIKKRLEKYSDILSKEQIKKLSRRRYTGWGRLSAKLLNGIRDKETN | |
| | KTILDYLIEDDNSNRNFMQLIHDDNLSFKEEIEKAQVIDDTESLHEVIANLA | |
| | GSPAIKKGILQSLKIVDEIVKVMGRYAPKNIVVEMARENQTTQKGQKNSRER | |
| | MKRLQEAMKEFGKDLLKEYPTDNTKLQNDKLYLYYLQNGKDMYTGEALDIDN | |
| | LSDYDVDHIVPQSFLKDDSIDNRVLVSSKEARGKSDDVPSIDIVRKMIGFWK | |
| | KLLDAKLITQRKYDNLTKGERGELTELDKAGFIKRQLVETRQITKHVAQILD | |
| | ERFNAEVDDDGKLIRKTKIVTLKSKLTSQFRKEFGLYKVREINNYHHAHDAY | |
| | LNAVVAKALIKVYPKLESEFVYGDYPVFDVKKEKRESKREIGKATQKKFFYS | |
| | NLMNMFKSDVKLADDSVVEKDIVDFNDETGEILWDKDKHISTIKKVLSMPQV | |
| | NIVKKTEVQTGGFSKESILPKRDSDKLIARKKDWNTEKYGGFDSPTVAYSVV | |
| | VIADIEKGKAKKLKTIKEIIGITIMERSAFEQDEVAFLENKGYQNIQENNLV | |
| | KLPKYSLYELENGRKRLLASAGELQKGNELALPNHYVELLYHAKRYEKIKRE | |
| | NDESEYSENYLQEHREEFNDLLDQVKEFAERYTLADANLEKIKKLFEENEEA | |
| | DLEELAKSFVNLLSFTAMGAPAAFKFFGKNIDRKRYTSIKELLNATIIHQSI | |
| | TGLYETRIDLSKLGED | |
| CasEnd-262 | MKKPYSIGLDIGTNSVGWAVVTDDYKVPSKKMKVLGNTDRSSIKKNLLGALL | 222 |
| | FDSGETAEATRLKRTARRRYTRRKNRILYLQEIFAEEMNKVDESFFHRLEDS | |
| | FLVEEDKRGERHPIFGNIVEEVKYHEEFPTIYHLRKHLADSKEKADLRLVYL | |
| | ALAHIIKFRGHFLIEGDLDTRNTDIQELFKEFLKVYDNTFENSHLSEETADV | |
| | EEILTDKLSKSAKKDKLLKLFPNEKSNGFFAEFLKLIVGNQADFKKHFSLSE | |
| | KAKLQFSKDTYEEDLETLLGQIGDEYADVFVAAKKLYDSILLSGILTVTDLS | |
| | TKAPLSASMVQRYEEHHEDLTKLKKFIRKKLPEKYKEFFFDTSKNGYAGYID | |
| | GGTKQEDFYKYLKKLLSKIEGSEYFLEKIEREDFLRKQRTFDNGSIPHQIHL | |
| | QELRAIIRRQGEYYPFLKENQDKIEQILTFRIPYYVGPLARGNSRFAWASRK | |
| | SDETITPWNFEEIIDKESSAEAFITRMTNNDLYLPEEKVLPKHSLLYEKFTV | |
| | YNELTKVKYITEQMGETEFFDANMKQEIFDGVFKKYRKVTKKKLINFLEKEF | |
| | DEFRIVDLSGVEKAFNASLGTYHDLLKILGDKEFLDDPANEKILEDIIQTLT | |
| | LFEDREMIKKRLSKYRDLFTKAQLKKLERRHYTGWGRLSAKLINGIRDKETG | |
| | KTILDYLIDDGRSNRNFMQLIHDDALSFKEEIAKAQVIGESESLHEVVAELA | |
| | GSPAIKKGILQSLKIVDELVKVMGRYNPENIVVEMARENQTTAKGQRNSRER | |
| | LKGLEDSMKELGSDILKEYPVDNSQLQNDRLYLYYLQNGKDMYTGEALDIDN | |
| | LSQYDVDHIIPQSFIKDDSIDNRVLVSSAKARGKSDDVPSKEVVHKMKPFWK | |
| | KLLDAKLISQRKYDNLTKAERGGLTDDDKAGFIKRQLVETRQITKHVAQILD | |
| | ERFNEEKDENNKLIRKVKIVTLKSKLVSQFRKEFELYKVREINDYHHAHDAY | |
| | LNAVVAKALITKYPKLEPEFVYGDYPKYNSYKLVSYSNEERGKATSKMFFYS | |
| | NLMNFFKKDVKLADGNVVERPDIEVNDETGEIAWDKTKHISTVKKVLSMPQV | |
| | NIVKKTEVQTGGFSKESILPKRNSDKLIARKKKWDTKKYGGFDSPIVAYSVL | |
| | VVADIKKGKKKKLKTVKEIVGITIMEKSTFEKDPIAFLEDKGYQNIREENII | |
| | KLPKYSLFELENGRKRLLASAGELQKGNELALPQHLVTLLYHAKRIEEFDEK | |
| | EEDEPEHLNYVMKHRSEFKELFDQVSEFSERYILADKNLEKIEELYDQNESA | |
| | DIKELASSFINLLTFTALGAPADFKFFGGDIDRKRYTSTTEILNATLIHQSI | |
| | TGLYETRIDLSKLGED | |
| CasEnd-263 | MDKPYSIGLDIGTNSVGWAVVTDEYKVPSKKFKVLGNTDRHSIKKNLLGALL | 223 |
| | FDSGETAEATRLKRTARRRYTRRKNRIRYLQEIFAEEMAKVDDSFFHRLEES | |
| | FLVEEDKRGERHPIFGNIVDEVAYHEEYPTIYHLRKHLADSDEKADLRLIYL | |
| | ALAHIIKFRGHFLIEGDLNAENSDVQKLFQALVETYDQTFEESPLSEETVDA | |
| | EVILTAKVSKSRRLENLIKQFPNEKKNGLFGNLVALSLGLKPNFKTNFELSE | |
| | DAKLQFSKDTYDEDLENLLGQIGDQYADLFAAAKNLSDAILLSGILTVNTEI | |
| | TKAPLSASMIKRYDEHHQDLTLLKAFIRQQLPEKYKEIFFDKSKNGYAGYID | |
| | GGASQEDFYKYIKPILSKMDGSEYFLDKINREDFLRKQRTFDNGSIPHQIHL | |
| | QELHAILRRQEEYYPFLKENQEKIEKILTFRIPYYVGPLARGNSRFAWMKRK | |
| | SDETITPWNFEEVVDKEASAQAFIERMTNNDKNLPNEKVLPKHSLLYEMFTV | |
| | YNELTKVKYVTEGMRKPAFFSSEQKQEIVDLLFKKNRKVTKKKLLEYLFKKF | |
| | DEFRSVDISGVEKAFNASLGTYHDLLKIIKDKEFLDNEENQDILEDIVLTLT | |
| | LFEDREMIEQRLSKYADLFDKKVLKKLKRRRYTGWGRLSAKLINGIRDKQSG | |
| | KTILDYLKDDGFANRNFMQLIHDDSLSFKEEIAKAQVIGKNDSLHEVIANLA | |
| | GSPAIKKGILQSLKIVDELVKVMGRYNPENIVIEMARENQTTQKGQRNSRER | |
| | LKRLEEGIKELGSNILKEHPVENTQLQNDKLYLYYLQNGKDMYTGEELDIDR | |
| | LSDYDVDHIIPQSFIKDDSIDNRVLVSSAKARGKSDDVPSEEVVKKMKGFWH | |
| | KLLKAKLISQRKFDNLTKAERGGLSELDKAGFIKRQLVETRQITKHVAQILD | |
| | SRFNTKYDENDKLIRDVKIITLKSKLVSQFRKDFKFYKVREINDYHHAHDAY | |
| | LNAVVGTALIKKYPKLESEFVYGDYPVYDSYKMIAKSEQEIGKATAKYFFYS | |
| | NIMNFFKTDITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VIADIEKGKTKKLKTVKELVGITIMERSSFEKNPILFLEDKGYQNIQKDNII | |
| | KLPKYSLFELENGRKRLLASAGELQKGNELALPQHYVTLLYHAKHYEKLKES | |
| | PEDNEKHLEYVIKHRDEFDEILDQISEFSKRYILADKNLEKIKELYSKNREA | |
| | DISELAKSFINLLTFTALGAPAAFKFLGADIDRKRYTSTTEILDATLIHQSI | |
| | TGLYETRIDLSQLGGD | |
| CasEnd-264 | MDKKYSIGLDIGTNSVGWAVITDEYKVPSKKFKVLGNTDRHSIKKNLIGALL | 224 |
| | FDSGETAEATRLKRTARRRYTRRKNRICYLQEIFSNEMAKVDESFFHRLEES | |
| | FLVEEDKKGERHPIFGNIVEEVAYHEKYPTIYHLRKHLADSTEKADLRLVYL | |
| | ALAHIIKFRGHFLIEGDLNIENTDVQKLFEQFVQVYDKTFEESHLEEETIDA | |
| | KAILTEKLSKSRRLENLIAQFPGEKKNGLFGNLIALSLGLTPNFKANFQLSE | |
| | DAKLQLSKDTYDEDLENLLGQIGDEYADLFVAAKNLYDAILLSGILTVNDLS | |
| | TKAPLSASMIKRYDEHHQDLTLLKAFVREQLPEKYKEIFFDSTKNGYAGYID | |
| | GGASQEEFYKYIKKILSKIDGSEYFLDKIEREDFLRKQRTFDNGSIPHQIHL | |
| | QELHAILRRQEAYYPFLKENQEKIEKILTFRIPYYVGPLARGNSRFAWASRK | |
| | SDETITPWNFEEVVDKEASAEAFIERMTNFDKNLPEEKVLPKHSLLYEKFTV | |
| | YNELTKVKYVTEGMGKAQFFDANQKQEIFDGLFKKERKVTKKKLLDFLDKEF | |
| | DEFRIVDISGVEDAFNASLGTYHDLLKIIKDKDFLDDEENEDILEDIILTLT | |
| | LFEDREMIEKRLSKYEDLFTKKVLKQLERRRYTGWGRLSKKLINGIRDKESG | |
| | KTILDYLISDGHANRNFMQLIHDDSLSFKEEIKKAQVKGEVDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKKKKLKTVKELVGITIMERSSFEKDPVAFLEKKGYQNIQEDLII | |
| | KLPKYSLFELENGRKRLLASAGELQKGNELALPQKYVTLLYHAKHYEKLKEK | |
| | PENIEEHLEYVEQHRDEFDEIFEQIEEFSKRYVLADKNLEKILELYAKNENF | |
| | SIEELAKSFINLLTFTALGAPAAFKFFGETIDRKRYTSTKECLNATLIHQSI | |
| | TGLYETRIDLSKLGGD | |
| CasEnd-265 | MDKKYSIGLDIGTNSVGWAVITDEYKVPSKKFKVLGNTDRHSIKKNLIGALL | 225 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVAKIEKGKAKKLKTVKELVGITIMERSAFEKDPVAFLEDKGYQNIQKDLFI | |
| | KLPKYSLFELENGRKRLLASAGELQKGNELALPQHYVTLLYHAKNYEKLKGS | |
| | PEDEKEHLIYIEEHREEFDEILDQIIEFSERYILKDANLEKIKELYEKNFEA | |
| | SIEELATSFINLLTFTALGAPAAFKFFGTDIDRKRYTSTKEILNSTLIHQSI | |
| | TGLYETRIDLSKLGGD | |
| CasEnd-266 | MDKKYSIGLDIGTNSVGWAVITDEYKVPSKKFKVLGNTDRHSIKKNLLGALL | 226 |
| | FDSGETAEATRLKRTARRRYTRRKNRIRYLQEIFAEEMSKVDDSFFHRLEES | |
| | FLVEEDKRGERHPIFGNIVDEVAYHEKYPTIYHLRKHLADSTEKADLRLIYL | |
| | ALAHIIKFRGHFLIEGKLNSENTDVQKLFIQFVQTYDQLFEESHLSEETVDA | |
| | EAILTEKLSKSRRLENLIKQFPGEKKNGLFGNLLALSLGLTPNFKSNEDLSE | |
| | DAKLQFSKDTYDEDLENLLAQIGDQYADLFVAAKNLSDAILLSGILTVNTEI | |
| | TKAPLSASMIKRYDEHHQDLTLLKKFVRQQLPEKYKEIFFDQSKNGYAGYID | |
| | GGASQEDFYKYIKPILSKLDGSEYFLDKIEREDFLRKQRTFDNGSIPHQIHL | |
| | QELHAILRRQEEFYPFLKENQEKIEKILTFRIPYYVGPLARGNSRFAWMSRK | |
| | SDETITPWNFEEVVDKEASAEAFIERMTNVDKNLPEEKVLPKHSLLYEKFTV | |
| | YNELTKVKYITEGMRKPAFLSAEQKEEIVDLLFKKNRKVTVKKLKEFLFKKI | |
| | ECFDSVEISGVEDRFNASLGTYHDLLKIIKDKAFLDNEENEEILEDIVLTLT | |
| | LFEDREMIEQRLSKYADLFDKKVLKKLKRRRYTGWGRLSAKLINGIRDKQSG | |
| | KTILDFLIDDGFANRNFMQLIHDDSLTFKEEIQKAQVIGETDSLHEVIANLA | |
| | GSPAIKKGILQSLKIVDELVKVMGRHAPENIVIEMARENQTTQKGQKNSRER | |
| | LKRLEEGIKELGSQILKEHPVDNTQLQNDKLYLYYLQNGKDMYTGEELDIDR | |
| | LSDYDVDHIIPQSFIKDDSIDNRVLTSSSKARGKSDNVPSIEVVKKMKSFWR | |
| | QLLNAKLISQRKFDNLTKAERGGLSELDKAGFIKRQLVETRQITKHVAQILD | |
| | SRFNTKYDENDKLIREVKIITLKSKLVSDFRKDFELYKVREINDYHHAHDAY | |
| | LNAVVGTALIKKYPKLESEFVYGDYKVYDSRKMIAKSEQEIGKATAKYFFYS | |
| | NIMNFFKSEVTLANGEIRKRPLIETNEETGEIVWDKEKDFATVKKVLSMPQV | |
| | NIVKKTEVQTGGFSKESILPKRNSDKLIARKKDWDTKKYGGFDSPTVAYSVL | |
| | VIAKVEKGKTKKLKTVKELVGITIMERSAFEKDPVAFLEKKGYQNIRKDNII | |
| | KLPKYSLFELENGRKRLLASAGELQKGNELALPNKYVTLLYHAKHYEKLKES | |
| | PEDNEKKLEYVKQHRDEFDEILDQISEFSERYILADKNLEKIQELYKQNREA | |
| | DIEELAESFINLFTFTALGAPAAFKFFDTTIDRKRYTSTTEILNATLIHQSI | |
| | TGLYETRIDLSQLGGD | |
| CasEnd-267 | MDKSYSIGLDIGTNSVGWAVITDEYKVPSKKFKVLGNTDRQSIKKNLLGALL | 227 |
| | FDSGETAEATRLKRTARRRYTRRKNRIRYLQEIFAEEMSKVDDSFFHRLEES | |
| | FLVEEDKKGERHPIFGNIVDEVAYHEKYPTIYHLRKKLADTTDKADLRLIYL | |
| | ALAHIIKFRGHFLIEGDLNPENSDVAKLFIQLVQTYNQLFEENPIDTSGVDA | |
| | KAILSAKLSKSRRLENLIALFPGEKKNGLFGNLIALSLGLTPNFKSNEDLTE | |
| | DAKLQLSKDTYDEDLDNLLGQIGDQYADLFLAAKNLSDAILLSDILTVNDES | |
| | TKAPLSASMIKRYDEHHQDLTLLKALVRKQLPEKYKEIFFDESKNGYAGYID | |
| | GGASQEEFYKYIKPILEKMDGAEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQAFIERMTNFDKNLPNEKVLPKHSLLYEYFTV | |
| | YNELTKVKYVTEGMRKPEFLSGEQKEEIVDLLFKTNRKVTVKQLKEDYFKEI | |
| | ECFDSVEISGVEDRFNASLGTYHDLLKIIKDKDFLDNEENEKILEDIILTLT | |
| | LFEDREMIEQRLEKYAHLFDKKVMKQLKRRRYTGWGRLSRKLINGIRDKQSG | |
| | KTILDFLKSDGFANRNFMQLIHDDSLTFKEEIQKAQVVGQGESLHEQIANLA | |
| | GSPAIKKGILQTIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTEYDENDKLIREVKIITLKSKLVSDFRKDFQFYKVREINDYHHAHDAY | |
| | LNAVVGTALIKKYPKLESEFVYGDYKVYDVRKMIRKSEKEIGKATAKYFFYS | |
| | NIMNFFKTEVTLANGEIRKRPLIETNEETGEIVWDKEKDFATVRKVLSMPQV | |
| | NIVKKTEVQTGGFSKESILPKRDSDKLIARKKDWDPKKYGGFDSPTVAYSVL | |
| | VVAKIEKGKSKKLKSVKELVGITIMERSSFEKDPVAFLEAKGYKNIQKDLII | |
| | KLPKYSLFELENGRKRLLASAGELQKGNELALPNKYVRFLYLAHHYEKLKGK | |
| | PEDNENKLEYVEQHRKYFDEILEQIKEFSERYILADKNLDKIKSTYAKNRDK | |
| | PINELAENFIHLFTLTALGAPAAFKFEDTTIDRKRYTSTSEVLNATLIHQSI | |
| | TGLYETRIDLSQLGGD | |
| CasEnd-268 | MDKKYSIGLDIGTNSVGWAVITDEYKVPSKKFKVLGNTDRKSIKKNLLGALL | 228 |
| | FDSGETAEATRLKRTARRRYTRRKNRILYLQEIFAEEMAKVDDSFFHRLEES | |
| | FLVEEDKRFERHPIFGNIVDEVAYHEKYPTIYHLRKKLADSDEKADLRLVYL | |
| | ALAHIIKFRGHFLIEGDLNAENTDVQKLFKQLVQTYNQTFEESHLEEEGVDA | |
| | EAILTEKLSKSRRLENLIAQFPGEKKNGLFGNLIALSLGLTPNFKSNEDLSE | |
| | DAKLQFSKDTYDEDLENLLGQIGDEYADLFLAAKNLYDAILLSGILTVDDES | |
| | TKAPLSASMVKRYDEHHQDLTLLKAFIRKQLPEKYKEIFFDQSKNGYAGYID | |
| | GGASQEEFYKYIKKILEKIDGSEEFLDKIEREDFLRKQRTFDNGSIPHQIHL | |
| | QELHAILRRQEKYYPFLKENQEKIEKILTFRIPYYVGPLARGNSRFAWMSRK | |
| | SDETITPWNFEEVVDKEASAEKFIERMTNFDKNLPNEKVLPKHSLLYEKFTV | |
| | YNELTKVKYVTEGGRKPEFFDAEQKQEIFDLLFKKNRKVTKKQLKEYLFKEF | |
| | DEFDSVEISGVEDRFNASLGTYHDLLKIIKDKDFLDNEENEKILEDIILTLT | |
| | LFEDREMIKERLEKYADLFDKKQLKKLKRRRYTGWGRLSAKLINGIRDKQSG | |
| | KTILDYLIDDGFANRNFMQLIHDDSLTFKEEIEKAQVIGDGDSLHELIANLA | |
| | GSPAIKKGILQSLKIVDELVKVMGRHAPENIVIEMARENQTTQKGQKNSRER | |
| | MKRLEEAIKELGSKILKEHPVENTQLQNDKLYLYYLQNGKDMYTGEELDIDR | |
| | LSDYDVDHIVPQSFLKDDSIDNRVLTSSKKARGKSDDVPSEEVVKKMKNFWR | |
| | KLLEAKLITQRKFDNLTKAERGGLTELDKAGFIKRQLVETRQITKHVAQILD | |
| | SRFNTKYDENDKLIRDVKIITLKSKLVSQFRKDFELYKVREINNYHHAHDAY | |
| | LNAVVGTALIKKYPKLESEFVYGDYKVYDVRKMIAKSEQEIGKATAKYFFYS | |
| | NIMNFFKTEVKLADGEIRKRPLIETNEETGEIVWDKEKDFATVRKVLSMPQV | |
| | NIVKKTEVQTGGFSKESILPKRDSDKLIARKKDWDTKKYGGFDSPTVAYSVL | |
| | VIAKVEKGKAKKLKTVKELVGITIMERSSFEKNPIAFLEDKGYKNVKKDLII | |
| | KLPKYSLFELENGRKRLLASAGELQKGNELALPQKYVILLYHAKHYEKLKGS | |
| | PEDNEQHQIYVEQHKEEFDEIFDQIIEFSKRYILADANLEKIKSLYEKNRDA | |
| | SIEELAESFINLLTFTALGAPAAFKFFGTTIDRKRYTSTKEILNATLIHQSI | |
| | TGLYETRIDLSKLGGD | |
| CasEnd-269 | MDKKYSIGLDIGTNSVGWAVITDEYKVPSKKFKVLGNTDRKSIKKNLIGALL | 229 |
| | FDSGETAEATRLKRTARRRYTRRKNRICYLQEIFSNEMAKVDDSFFHRLEES | |
| | FLVEEDKRNERHPIFGNIVEEVAYHEKYPTIYHLRKKLADSDEKADLRLVYL | |
| | ALAHIIKFRGHFLIEGDLDSENSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVSDET | |
| | TKAPLSASMVKRYDEHHQDLTLLKQFIREQLPEKYKEIFFDQSKNGYAGYID | |
| | GGASQEEFYKYLKKILEKIDGSEEFLDKIEREDFLRKQRTFDNGSIPHQIHL | |
| | QELHAIIRRQEKYYPFLKENQEKIEKILTFRIPYYVGPLARGNSRFAWLSRK | |
| | SDETITPWNFEEVVDKEKSAQAFIERMTNFDKNLPDEKVLPKHSLLYEKFTV | |
| | YNELTKVKYVTEGMRKPEFFSAEQKQEIVDLLFKKYRKVTKKQLKEYLFKEF | |
| | DCFDIVEISGVEDRFNASLGTYHDLLKILKDKEFLDNEENEEILEDIILTLT | |
| | LFEDREMIKKRLEKYADLFDKKQLKKLERRRYTGWGRLSAKLINGIRDKQTG | |
| | KTILDYLIADGFANRNFMQLIHDDSLTFKEEIEKAQVIGKGDSLHELIANLA | |
| | GSPAIKKGILQSLKIVDELVKVMGRYAPENIVVEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKYDENGKLIRDVKIITLKSKLVSDERKDFGLYKVREINNYHHAHDAY | |
| | LNAVVGTALIKKYPKLESEFVYGDYKVYDVRKMIAKSEKEIGKATAKYFFYS | |
| | NIMNFFKTDVTLANGEIRKRPLIEVNEETGEIVWDKEKDIATVRKVLSMPQV | |
| | NIVKKTEVQTGGFSKESILPKRDSDKLIARKKDWDTKKYGGFDSPTVAYSVL | |
| | VIADVEKGKAKKLKTVKELVGITIMERSAFEKDPVAFLEDKGYKNIQKDLLI | |
| | KLPKYSLFELENGRKRLLASAGELQKGNELALPQHYVTLLYHAKHYEKLKGK | |
| | PEDNEKKQLYVEEHKHYFDEILDQIEEFAKRYILADANLEKIKELYEKNRDA | |
| | SIEELAENFIHLLTFTALGAPAAFKFFGKTIDRKRYTSTKEILNATLIHQSI | |
| | TGLYETRIDLSKLGGD | |
| CasEnd-270 | MDKKYSIGLDIGTNSVGWAVITDEYKVPSKKFKVLGNTDRHSIKKNLIGALL | 230 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKQLVRKQLPEKYKEIFFDQSKNGYAGYID | |
| | GGASQEEFYKYIKPILEKMDGTEELLAKLEREDLLRKQRTFDNGSIPHQIHL | |
| | GELHAILRRQEKYYPFLKENREKIEKILTFRIPYYVGPLARGNSRFAWMTRK | |
| | SDETITPWNFEEVVDKGASAEKFIERMTNFDKNLPEEKVLPKHSLLYEYFTV | |
| | YNELTKVKYVTEGMRKPEFLSGEQKKEIVDLLFKKYRKVTVKQLKEYYFKEF | |
| | ECFDIVEISGVEDRFNASLGTYHDLLKIIKDKDFLDNEKNEEILEDIVLTLT | |
| | LFEDREMIKERLEKYADLFDKKVMKQLKRRRYTGWGRLSRKLINGIRDKQSG | |
| | KTILDFLKADGFANRNFMQLIHDDSLTFKEEIEKAQVGGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKIEKGKAKKLKTVKELLGITIMERSAFEKNPVAFLEDKGYQEVKKELII | |
| | KLPKYSLFELENGRKRMLASAGELQKGNELALPSKYVNFLYLASNYEKLKGK | |
| | PEDNEQKQIYVEQHKEYLDEIIDQISEFSKRVILADANLEKVKSAYEKHREK | |
| | SIEEQAENIIHLFTLTDLGAPAAFKYFDTTIDRKRYTSTKEVLNATLIHQSI | |
| | TGLYETRIDLSQLGGD | |
| CasEnd-271 | MDKPYSIGLDIGTNSVGWAVITDEYKVPSKKFKVLGNTDRHSIKKNLLGALL | 231 |
| | FDSGETAEATRLKRTARRRYTRRKNRIRYLQEIFAEEMNKVDDSFFHRLEES | |
| | FLVEEDKEEERHPIFGNIVDEVAYHEKYPTIYHLRKHLADSTEKADLRLIYL | |
| | ALAHIIKFRGHFLIEGDLNSENTDVQKLFIQFVQVYDNLFEESHLEEETVDA | |
| | EAILTEKLSKSRRLENLIKQFPNEKKNGLFGNLLALSLGLTPNFKSNEDLAE | |
| | DAKLQFSKDTYEEDLENLLAQIGDEYADLFLAAKNLSDAILLSGILTVNTEI | |
| | TKAPLSASMIKRYDEHHQDLTLLKKFVRQQLPEKYKEIFFDESKNGYAGYID | |
| | GGASQEEFYKYIKPILSKLDGTEYFLAKIEREDFLRKQRTFDNGSIPHQIHL | |
| | QELHAILRRQEDFYPFLKENQEKIEKILTFRIPYYVGPLARGNSRFAWLSRK | |
| | SDETITPWNFEEVVDKEASAEAFIERMTNFDKNLPNEKVLPKHSLLYEKFTV | |
| | YNELTKVKYVTEGMRKPAFFSAEQKKEIVDLLFKKNRKVTVKKLKEHLFKEF | |
| | ECFDIVEISGVEDAFNASLGTYHDLLKIIKDKDFLDNEENEDILEDIVLTLT | |
| | LFEDREMIEKRLSKYAHLFDKKVLKKLKRRRYTGWGRLSAKLINGIRDKQTG | |
| | KTILDYLIDDGFANRNFMQLIHDDSLTFKEEIKKAQVIGQGDSLHETIANLA | |
| | GSPAIKKGILQSLKIVDELVKVMGRHNPENIVIEMARENQTTQKGQKNSRER | |
| | LKRLEEAIKKLGSQILKEHPVENTQLQNDKLYLYYLQNGKDMYTGEELDIDR | |
| | LSDYDVDHIIPQSFIKDDSIDNRVLTSSAKARGKSDDVPSEEVVKKMKAYWR | |
| | QLLNAKLISQRKFDNLTKAERGGLSELDKAGFIKRQLVETRQITKHVAQILD | |
| | SRFNTKYDENDKLIREVKIITLKSKLVSDFRKDFGFYKVREINDYHHAHDAY | |
| | LNAVVGTALIKKYPKLESEFVYGDYKVYNSYKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVADVEKGKAKKLKTVKELVGITIMERSAFEKDPIAFLEDKGYQNIQKENII | |
| | KLPKYSLFELENGRKRLLASAGELQKGNELALPNKYVTLLYHAKNYEKLKGS | |
| | PEDNEKHLEYVEQHRYEFDEILDQISEFSERYILADKNLEKIEELYAENEDK | |
| | SIEELAESFINLFTFTALGAPAAFKFFDKTIDRKRYTSTTEILNATLIHQSI | |
| | TGLYETRIDLSQLGGD | |
| CasEnd-272 | MDKPYSIGLDIGTNSVGWAVITDEYKVPSKKFKVLGNTDRHSIKKNLIGALL | 232 |
| | FDSGETAEATRLKRTARRRYTRRKNRILYLQEIFAEEMNKVDDSFFHRLEES | |
| | FLVEEDKRYERHPIFGNIVDEVAYHENYPTIYHLRKKLADSTEKADLRLIYL | |
| | ALAHIIKFRGHFLIEGDLDSENTDVQKLFKALVQTYNNTFEESHLEEATVDA | |
| | KSILTDKLSKSRRLENLIAQFPNEKKNGLFGNLIALALGLTPNFKSNEDLAE | |
| | DAKLQFSKDTYDEDLENLLTQIGDQYADLFLAAKNLYDAILLSGILTVNTEI | |
| | TKAPLSASMIKRYDEHHQDLTLLKKLVREQLPEKYKEIFFDDTKNGYAGYID | |
| | GGASQEEFYKYIKNILSKLDGTEYFLAKIEREDFLRKQRTFDNGSIPHQIHL | |
| | EELHAILRRQEEFYPFLKENREKIEKILTFRIPYYVGPLARGNSRFAWMSRK | |
| | SDETITPWNFEEVVDKEASAEAFIERMTNNDKNLPNEKVLPKHSLLYEKFTV | |
| | YNELTKVKYITEGMRKPAFLSAEQKKDIVDLLFKKDRKVTVKKLKEFLFKKI | |
| | ECLDSVEISGVEDKFNASLGTYHDLLKIIKDKEFLDNEENEEILEDIVLTLT | |
| | LFEDREMIKQRLAKYAHLFDKKVLKKLKRRRYTGWGRLSRKLINGIRDKQTG | |
| | KTILDYLKDDGFANRNFMQLIHDDSLTFKEEIKKAQVTGQGDSLHEVIANLA | |
| | GSPAIKKGILQSLKIVDELVKVMGRHNPENIVIEMARENQTTQKGQKNSRER | |
| | LKRLEEAIKKLGSKILKEHPVENTQLQNDKLYLYYLQNGRDMYTGEELDIDR | |
| | LSDYDVDHIIPQSFIKDDSIDNRVLTSSAEARGKSDNVPSIEVVKKMKSYWS | |
| | KLLNSKLISQRKFDNLTKAERGGLSELDKAGFIKRQLVETRQITKHVAQILD | |
| | SRFNTKYDENDKLIRKVKIITLKSKLVSDFRKDFGFYKVREINDYHHAHDAY | |
| | LNAVVGTALIKKYPKLESEFVYGDYKVYNSRKMIAKSEQEIGKATAKYFFYS | |
| | NIMNFFKSEITLANGEIRKRPLIETNEETGEIAWNKVKHFATVRKVLSMPQV | |
| | NIVKKTEVQTGGFSKESILPKRNSDKLIARKKDWDTKKYGGFDSPTVAYSVL | |
| | VVADREKGKAKKLKTVKELVGITIMERSTFEKDPIAFLEGKGYQNIQKELII | |
| | KLPKYSLFELENGRKRLLASAGELQKGNELALPQKYVTLLYHASHYEKLKES | |
| | PEDNEKHLEYVEQHREEFDEIFDQISEFSKRYILADKNLEKILSLYDKNRQS | |
| | SIEELAESFINLFTFTALGAPAAFKFFNKTIDRKRYTSTSEILNATLIHQSI | |
| | TGLYETRIDLSQLGGD | |
| CasEnd-273 | MKKPYSIGLDIGTNSVGWAVVTDDYKVPAKKMKVLGNTDKSHIKKNLLGALL | 233 |
| | FDSGNTAEDRRLKRTARRRYTRRRNRILYLQEIFSEEMGKVDDSFFHRLEDS | |
| | FLVPEDKRGERHPIFGNLEEEVKYHENFPTIYHLRKYLADNPEKADLRLVYL | |
| | ALAHIIKFRGHFLIEGKFDTRNNDVQRLFQEFLAVYDNTFENSSLQEQNVQA | |
| | EEILTDKISKSAKKDRVLKLFPNEKSNGRFAEFLKLIVGNQADFKKHFELEE | |
| | KAPLQFSKDTYEEDLEVLLAQIGDEYAELFLSAKKLYDSILLSGILTVTDVS | |
| | TKAPLSASMIQRYNEHQEDLTQLKQFIRQKLPDKYNEVFSDVSKNGYAGYID | |
| | GKTNQEAFYKYLKGLLNKIEGSGYFLDKIEREDFLRKQRTFDNGSIPHQIHL | |
| | QEMRAIIRRQAEFYPFLAENQDKIEKILTFRIPYYVGPLARGKSDFAWLSRK | |
| | SAEKITPWNFDEIVDKESSAEAFINRMTNYDLYLPNQKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASYGTYHDLLKILKDKDELDNSKNEKILEDIVLTLT | |
| | LFEDREMIRKRLENYSDLLTKEQLKKLERRHYTGWGRLSAKLIHGIRNKESR | |
| | KTILDYLIDDGNSNRNFMQLINDDALSFKEEIAKAQVIGETDNLNQVVSDIA | |
| | GSPAIKKGILQSLKIVDELVKIMGGHQPENIVVEMARENQFTNQGRRNSQQR | |
| | LKGLTDSIKEFGSQILKEHPVDNSQLQNDRLFLYYLQNGRDMYTGEELDIDY | |
| | LSQYDIDHIIPQAFIKDNSIDNRVLVSSKEARGKSDDVPSKDVVRKMKSYWS | |
| | KLLSAKLITQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLGKYPQLEPEFVYGDYPKFNSHKFVAKDAKEEKKATAKKFFYS | |
| | NIMNFFKSDDKLADGQIVERPMVERNDENGEIIWDKTKHISTVKKVLSYPQV | |
| | NIVKKVEEQTGGFSKESILPKGNSDKLIPRKTKWDTKKYGGFDSPIVAYSVL | |
| | VIADIEKGKSKKLKTVKALVGITIMEKMTFEKDPVAFLERKGYRNIQEENII | |
| | KLPKYSLFELENGRKRLLASARELQKGNEIVLPNHLVTLLYHAKNIHKVDEK | |
| | AEDIPKHLDYVDKHRAEFKELLDVVSNFSKKYTLAEGNLEKIKELYAQNNSA | |
| | DIKELASSFINLLTFTALGAPATFKFFDKNIDRKRYTSTTEILNATLIHQSI | |
| | TGLYETRIDLSKLGGD | |
| CasEnd-274 | MKKPYSIGLDIGTNSVGWAVVTDEYKVPSKKFKVLGNTDRQSIKKNLLGALL | 234 |
| | FDSGETAEATRLKRTARRRYTRRKNRILYLQEIFAEEMNKVDESFFHRLEES | |
| | FLVEEDKRGERHPIFGNIVEEVAYHEKYPTIYHLRKHLADSTEKADLRLVYL | |
| | ALAHIIKFRGHFLIEGDLNSENTDVQKLFKDFVEVYDKTFEESHLSEETVDA | |
| | ESILTEKVSKSRRLENLIKQFPNEKKNGLFGNLLALSLGLQPNFKTNFQLSE | |
| | DAKLQFSKDTYEEDLENLLGQIGDEYADLFTAAKNLYDAILLSGILTVNDES | |
| | TKAPLSASMVKRYDEHHQDLTKLKAFIRQNLPEKYKEIFFDKSKNGYAGYID | |
| | GGAKQEEFYKYLKNILSKIDGSEYFLDKIEREDFLRKQRTFDNGSIPHQIHL | |
| | QELKAILRRQGEYYPFLKENQEKIEKILTFRIPYYVGPLARGNSRFAWASRK | |
| | SDETITPWNFDEVVDKEKSAEAFIERMTNNDKNLPEEKVLPKHSLLYEKFTV | |
| | YNELTKVKYVTEQMGKAEFFDANMKQEIFDGVFKKYRKVTKKKLLDFLDKEF | |
| | DEFRIVDISGVEKAFNASLGTYHDLKKILKDKSFLDNPENEKILEDIILTLT | |
| | LFEDREMIRKRLEKYADLFTKKQLKKLERRRYTGWGRLSAKLINGIRDKQSG | |
| | KTILDYLIDDGYANRNFMQLIHDDSLSFKEEIKKAQVIGESDSLHEVIADLA | |
| | GSPAIKKGILQSLKIVDELVKVMGRYNPENIVIEMARENQTTQKGQRNSRER | |
| | LKRLEESIKELGSDILKEHPVENTQLQNDKLYLYYLQNGKDMYTGEELDIDN | |
| | LSDYDVDHIIPQSFIKDDSIDNRVLTSSAKARGKSDDVPSEEVVRKMKSFWS | |
| | KLLKAGLISQRKFDNLTKAERGGLTELDKAGFIKRQLVETRQITKHVAQILD | |
| | ERFNTERDENNKLIRDVKIITLKSKLVSNFRKEFEFYKVREINDYHHAHDAY | |
| | LNAVVGKALIKKYPKLESEFVYGDYPVYNSYKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-275 | MDKSYSIGLDIGTNSVGWAVITDEYKVPSKKFKVLGNTDRKSIKKNLIGALL | 235 |
| | FDSGETAEATRLKRTARRRYTRRKNRICYLQEIFSNEMAKVDDSFFHRLEES | |
| | FLVEEDKRFERHPIFGNIVEEVAYHEKYPTIYHLRKKLVDSDEKADLRLIYL | |
| | ALAHMIKFRGHFLIEGDLNSDNSDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEHIANLA | |
| | GSPAIKKGILQTVKVVDELVKVMGRHKPKNIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTEYDENDKLIRDVKVITLKSKLVSDFRKDFGFYKVREINNYHHAHDAY | |
| | LNAVVGTALIKKYPKLESEFVYGDYPVYDVRKMIAKSSQEIGKATAKYFFYS | |
| | NIMNFFKSEITLANGTIRKRPLIESNEETGEIVWDKEKDFATVKKVLSMPQV | |
| | NIVKKTEVQTGGFSKESILPKRDSDKLIARKKDWDPRKYGGFDSPTVAYSVL | |
| | VVAKIEKGKTKKLKTVKELLGITIMERSAFEKDPVAFLEDKGYKDVKKNLII | |
| | KLPKYSLFELENGRKRMLASAGELQKGNELALPSHYVNFLYLASRYEKLKGK | |
| | EEDEKQKQIYVEKHLEYLKEIIDQISEFSERVILADANLEKVKKAYEEHSEK | |
| | SIEEQAENIIHLFTLTALGAPAAFKYFNVDIDRKRYTSTKEVLNATLIHQSI | |
| | TGLYETRIDLSKLGGD | |
| CasEnd-276 | MDKKYSIGLDIGTNSVGWAVITDEYKVPSKKFKVLGNTDRQSIKKNLIGALL | 236 |
| | FDSGETAEATRLKRTARRRYTRRKNRICYLQEIFSEEMSKVDDSFFHRLEES | |
| | FLVEEDKRHERHPIFGNIVDEVAYHEKYPTIYHLRKKLVDSTDKADLRLIYL | |
| | ALAHMIKFRGHFLIEGDLNPDNSDVDKLFIQLVQTYNQTFEENPISAETVDA | |
| | EAILTERLSKSRRLENLIAQLPNEKKNGLFGNLIALSLGLTPNFKSNFELSE | |
| | DAKLQLSKDTYDEDLENLLAQIGDQYADLFTAAKNLYDAILLSDILRVNTLI | |
| | TKAPLSASMIKRYDEHHQDLTLLKALVRQQLPEKYKEIFFDESKNGYAGYID | |
| | GGASQEEFYKFIKPILSKMDGTEYLLVKLEREDLLRKQRTFDNGSIPHQIHL | |
| | QELHAILRRQEDFYPFLKDNREKIEKILTFRIPYYVGPLARGNSRFAWMTRK | |
| | SDETITPWNFEEVVDEGASAQSFIERMTNFDKNLPNEKVLPKHSLLYEYFTV | |
| | YNELTKVKYVTEGMRKPAFLSGEQKKEIVDLLFKTNRKVTVKQLKEDLENEI | |
| | DCFDSVDISGVEDRFNASLGTYHDLLKIIKDKDFLDNEENEDILEDIVLTLT | |
| | LFEDREMIEKRLKTYANLFDKKVMKQLKRRRYTGWGRLSRKLINGIRDKQSG | |
| | KTILDFLKSDGFANRNFMQLIHDDSLTFKEDIQKAQVSGQTDSLHEQIANLA | |
| | GSPAIKKGILQTLKVVDELVKVMGRYKPENIVIEMARENQTTQKGQKNSRER | |
| | MKRIEEGIKELGSKILKEHPVENTQLQNEKLYLYYLQNGRDMYTDQELDINR | |
| | LSDYDVDHIVPQSFLKDDSIDNRVLTSSKKARGKSDDVPSEEVVKKMKSYWR | |
| | QLLNAGLITQRKFDNLTKAERGGLSELDKAGFIKRQLVETRQITKHVAQILD | |
| | SRMNTKYDENSKLIRDVKVITLKSKLVSDFRKDFQFYKVREINNYHHAHDAY | |
| | LNAVVGTALIKKYPKLESEFVYGDYKVYDSYRMVAKSDKEIGKATAKYFFYS | |
| | NIMNFFKSDVKLADGRIRERPQIETNEETGEIAWDKGRDFATVRKVLSMPQV | |
| | NIVKKTEVQTGGFSKESILPKRNSDKLIARKKDWDPKKYGGFDSPTVAYSVL | |
| | VVADVEKGKAKKLKSVKELLGITIMERSSFEKNPVDFLEAKGYQNIQEDKII | |
| | KLPKYSLFELENGRKRLLASAGELQKGNELALPQKYVTFLYHAKHYEKLKGK | |
| | PEDLEKHQLFVEQHRHYFDEILEQIIEFSERYILADKNLEKIKELFAEHEEA | |
| | SIREQASNIINLETFTNLGAPAAFKYFDTDIDRKRYTSTTEILNATLIHQSI | |
| | TGLYETRIDLSQLGGD | |
| CasEnd-277 | MDKKYSIGLDIGTNSVGWAVITDEYKVPSKKFKVLGNTDRHSIKKNLIGALL | 237 |
| | FDSGETAEATRLKRTARRRYTRRKNRICYLQEIFSNEMAKVDDSFFHRLEES | |
| | FLVEEDKKGERHPIFGNIVDEVAYHEKYPTIYHLRKKLADSTEKADLRLIYL | |
| | ALAHIIKFRGHFLIEGDLNSENTDVQKLFKQLVQTYDQTFEESHLNEETVDA | |
| | KSILTEKLSKSRRLENLIKQFPNEKKNGLFGNLIALSLGLQPNFKSNEDLSE | |
| | DAKLQFSKDTYDEDLENLLGQIGDQYADLFVAAKNLYDAILLSGILTVNDES | |
| | TKAPLSASMIKRYDEHHQDLTLLKAFVRQQLPEKYKEIFFDETKNGYAGYID | |
| | GGASQEEFYKYIKPILEKMDGSEYFLEKIEREDFLRKQRTFDNGSIPHQIHL | |
| | QELHAILRRQEEYYPFLKENQEKIEKILTFRIPYYVGPLARGNSRFAWMTRK | |
| | SDETITPWNFDEVVDKEASAQAFIERMTNFDKNLPEEKVLPKHSLLYEKFTV | |
| | YNELTKVKYVTEQMGKPQFFDANLKQEIFDGLFKKNRKVTKKKLLDFLDKEF | |
| | DEFRIVDISGVEDAFNASLGTYHDLLKIIKDKEFLDNEENEDILEDIVLTLT | |
| | LFEDREMIEKRLSKYADLFDKKVLKQLERRRYTGWGRLSAKLINGIRDKQSG | |
| | KTILDYLIDDGFANRNFMQLIHDDSLTFKEEIKKAQVIGQGDSLHEHIANLA | |
| | GSPAIKKGILQTVKVVDELVKVMGRHKPENIVIEMARENQTTQKGQRNSRER | |
| | LKRLEEAIKELGSKILKEHPVENTQLQNDKLYLYYLQNGKDMYTGEELDIDR | |
| | LSDYDVDHIIPQSFIKDDSIDNRVLTSSAKARGKSDNVPSEEVVRKMKSEWS | |
| | KLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVAKIEKGKAKKLKTVKELVGITIMERSAFEKNPVAFLEDKGYQNIQEDLII | |
| | KLPKYSLFELENGRKRLLASAGELQKGNELALPQKYVTLLYHAKHYEKLKGK | |
| | PEDEEKHLEYVEQHRSEFDEILEQISEFSERYILADKNLEKILELYEQFENK | |
| | SIEELASSFINLLTLTALGAPAAFKFFGETIDRKRYTSTKEILNATLIHQSI | |
| | TGLYETRIDLSKLGGD | |
| CasEnd-278 | MKKKYSIGLDIGTNSVGWAVITDEYKVPSKKFKVLGNTDRQSIKKNLLGALL | 238 |
| | FDSGETAEATRLKRTARRRYTRRKNRIRYLQEIFSEEMSKVDDSFFQRLEES | |
| | FLVEEDKRHERHPIFGNIVEEVAYHEKYPTIYHLRKKLADSTQKADLRLIYL | |
| | ALAHMIKFRGHFLIEGDLNPENSDVQKLFIQLVQTYNQLFEENPINESGVDA | |
| | KAILSAKLSKSRRLENLIAQFPGEKKNGLFGNLIALSLGLTPNFKSNFDLAE | |
| | DAKLQLSKDTYDEDLDNLLAQIGDQYADLFAAAKNLSDAILLSGILTVNDEI | |
| | TKAPLSASMIKRYDEHHQDLTLLKNLVREQLPEKYKEIFFDDSKNGYAGYID | |
| | GGASQEEFYKFIKPILEKMDGTEYLLAKINREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQAFIERMTNFDKNLPTEKVLPKHSLLYEYFTV | |
| | YNELTKVKYVTEGMRKPEFLSSNQKKEIVDLLFKKSRKVTVKQLKEDYFKKI | |
| | ECFDSVEISGVEDRFNASLGTYHDLLKIIKDKDFLDNEENEEILEDIVLTLT | |
| | LFEDREMIEERLKKYANLFDKKVLKQLKRRRYTGWGRLSRKLINGIKDKQSG | |
| | KTILDFLKSDGFANRNFMQLIHDDSLTFKEEIQKAQVSGQGESLHEVIANLA | |
| | GSPAIKKGILQSIKIVDEIVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKRDENDKLIREVKIITLKSKLVSDFRKDFQFYKVREINNYHHAHDAY | |
| | LNAVVGTALIKKYPKLESEFVYGDYKVYDVRKKIAKSEQEIGKATAKYFFYS | |
| | NIMNFFKSEITLANGEIRKRPLIETNEETGEIVWNKEKDFATVKKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSSFEKDPVAFLEAKGYKEIQKDLII | |
| | KLPKYSLFELENGRKRLLASAGELQKGNELALPQKYVTFLYLAKHYEKLKGK | |
| | PEDLEKNLEYVEEHRDYFKEILEQIKEFSERYILADANLEKIKELYNEHEDY | |
| | EISELAENFIHLFTLTSLGAPAAFKFFDTTIDRKRYTSTKEILNATLIHQSI | |
| | TGLYETRIDLSQLGGD | |
| CasEnd-279 | MDKKYSIGLDIGTNSVGWAVITDEYKVPSKKFKVLGNTDRHSIKKNLIGALL | 239 |
| | FDSGETAEATRLKRTARRRYTRRKNRICYLQEIFSNEMAKVDDSFFHRLEES | |
| | FLVEEDKKDERHPIFGNIVDEVAYHEKYPTIYHLRKHLADSTEKADLRLVYL | |
| | ALAHIIKFRGHFLIEGDLNSENTDVQKLFKQLVQTYNRTFEESPLSEETVDA | |
| | EAILTEKLSKSRKLENLIAQFPNEKKNGLFGNLIALSLGLQPNFKSNFKLSE | |
| | DAKLQFSKDTYDEDLENLLGQIGDEYADLFLAAKNLYDAILLSGILTVNDES | |
| | TKAPLSASMIKRYDEHHQDLTLLKKFVREQLPEKYKEIFFDETKNGYAGYID | |
| | GGASQEEFYKYIKPLLEKVDGAEYFLEKIEREDFLRKQRTFDNGSIPHQIHL | |
| | GELHAILRRQEEFYPFLKENQEKIEKILTFRIPYYVGPLARGNSRFAWMSRK | |
| | SDETITPWNFEEVVDKEASAEAFIERMTNFDKNLPEEKVLPKHSLLYEKFTV | |
| | YNELTKVKYVTEGMGKPEFFSANQKQEIFDGLFKKNRKVTKKKLKEFLFKEF | |
| | EEFRIVDISGVEDAFNASLGTYHDLLKIIKDKDFLDNEENEDILEDIILTLT | |
| | LFEDREMIEKRLQKYADLFDKKQLKKLERRRYTGWGRLSKKLINGIRDKQSG | |
| | KTILDFLIDDGFANRNFMQLIHDDSLSFKEEIKKAQVIGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSRFEKNPIAFLEDKGYQNIQEDKII | |
| | KLPKYSLFELENGRKRLLASAGELQKGNELALPQKYVTLLYHAKNYEKLKEK | |
| | PEDIEKHLEYVEKHRDEFKEILSQIIEFSKRYILADKNLEKIKELFNQNENS | |
| | SISELASSFINLLTFTSLGAPAAFKFFGSTIDRKRYTSTKEILNATLIHQSI | |
| | TGLYETRIDLSKLGGD | |
| CasEnd-280 | MDKPYSIGLDIGTNSVGWAVVTDEYKVPSKKFKVLGNTDRHSIKKNLLGALL | 240 |
| | FDSGETAEATRLKRTARRRYTRRKNRIRYLQEIFAEEMNKVDDSFFHRLEES | |
| | FLVEEDKRNERHPIFGNIVEEVAYHEKYPTIYHLRKHLADSTEKADLRLIYL | |
| | ALAHIIKFRGHFLIEGDLDSENTDVQKLFSALVQVYNQLQEESPLSEETVDA | |
| | EAILTAKISKSRRLENLIALFPGEKKNGLFGNLIALSLGLTPNFKSNFELSE | |
| | DAKLQFSKDTYDEDLENLLAQIGDQYADLFLAAKNLYDAILLSGILTVKTEI | |
| | TKAPLSASMVKRYDEHHQDLTLLKDFIRQQLPEKYKEIFFDDSKNGYAGYID | |
| | GGAKQEEFYKYIKPILEKLDGSEDFLDKIEREDFLRKQRTFDNGSIPHQIHL | |
| | EELHAILRRQEEFYPFLKENQEKIEKILTFRIPYYVGPLARGNSRFAWMSRK | |
| | SDETITPWNFEEVVDKEASAQLFIERMTNFDKNLPNEKVLPKHSLLYEMFTV | |
| | YNELTKVKYVTEGMRKPAFFSSEQKKEIVDLLFKKYRKVTVKQLKNFLFKEF | |
| | ECFDIVEISGVEDRFNASLGTYHDLLKIIKDKDFLDNEENEDILEDIVLTLT | |
| | LFEDREMIEERLEKYADIFDKNVLKKLKRRRYTGWGRLSGKLINGIRDKQSG | |
| | KTILDYLIDDGFANRNFMQLIHDDSLTFKEEIAKAQVIGDTDSLHETIANLA | |
| | GSPAIKKGILQSLKIVDELVKVMGRHKPENIVIEMARENQTTQKGQKNSRER | |
| | LKRLEEAIKELGSKILKEHPVENTQLQNDKLYLYYLQNGRDMYTGEELDIDR | |
| | LSDYDVDHIIPQSFIKDDSIDNKVLTSSAKARGKSDNVPSIEVVKKMKPYWQ | |
| | QLLDAKLISQRKFDNLTKAERGGLSELDKAGFIKRQLVETRQITKHVAQILD | |
| | SRFNTKYDENDKLIRDVKIITLKSKLVSDFRKDFGFYKVREINDYHHAHDAY | |
| | LNAVVGTALIKKYPKLESEFVYGDYKVYNSRKMIAKSEQEIGKATAKYFFYS | |
| | NIMNFFKKDITLANGEIRKRPLIETNEETGEIVWDKEKDFATVKKVLSMPQV | |
| | NIVKKTEVQTGGFSKESILPKRNSDKLIARKKDWDTTKYGGFDSPTVAYSVL | |
| | VIAEIEKGKAKKLKTVKELVGITIMERSSFEKNPIAFLEAKGYKNIRKDLII | |
| | KLPKYSLFELENGRKRLLASAGELQKGNELALPQKYVTLLYHASHYEKLKES | |
| | PEDNPPKFEYVVQHKHEFDEILDQIEEFSERYILADKNLEKINELYEENRDA | |
| | SIEELAESFINLLTFTALGAPAAFKFFGQTIDRKRYTSTTEVLNATLIHQSI | |
| | TGLYETRIDLSQLGGD | |
| CasEnd-281 | MKKKYSIGLDIGTNSVGWAVITDEYKVPSKKFKVLGNTDRHSIKKNLLGALL | 241 |
| | FDSGETAEATRLKRTARRRYTRRKNRICYLQEIFSEEMSKVDDSFFHRLEES | |
| | FLVEEDKKGERHPIFGNIVDEVAYHEKYPTIYHLRKKLADSPEKADLRLIYL | |
| | ALAHMIKFRGHFLIEGDLNPENTDVDKLFIQLVQTYNQLFEENAIDASGVDA | |
| | KDILSAKLSKSRRLENLIAQFPGEKKNGLFGNLIALSLGLTPNFKSNEDLAE | |
| | DAKLQLSKDTYDEDLDNLLAQIGDQYADLFLAAKNLSDAILLSGILTVNDES | |
| | TKAPLSASMIKRYDEHHQDLTLLKALVREQLPEKYKEIFFDESKNGYAGYID | |
| | GGASQEEFYKYIKPILEKMDGTEELLAKLNREDFLRKQRTEDNGSIPHQIHL | |
| | GELHAILRRQEDFYPFLKENREKIEKILTFRIPYYVGPLARGNSRFAWMTRK | |
| | SEETITPWNFEEVVDKGASAQAFIERMTNFDKNLPTEKVLPKHSLLYEYFTV | |
| | YNELTKVKYVTEGMRKPEFFSGNQKEAIVDLLFKTNRKVTVKQLKEDYFKKI | |
| | DCFDIVEISGVEDRFNASLGTYHDLLKIIKDKDFLDNEENEDILEDIVLTLT | |
| | LFEDREMIEQRLKKYAHLFDKKVLKQLKRRRYTGWGRLSRKLINGIRDKQSG | |
| | KTILDFLKADGFANRNFMQLIHDDSLTFKEEIQKAQVSGQTDSLHETIANLA | |
| | GSPAIKKGILQSVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTERDENDKLIREVKIITLKSKLVSDFRKDFQFYKVREINDYHHAHDAY | |
| | LNAVVGTALIKKYPKLESEFVYGDYKVYDVRKMISKSEQEIGKATAKYFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VIAKVEKGKAKKLKSVKELVGITIMERSSFEKDPVAFLEDKGYKNVQKDLII | |
| | KLPKYSLFELENGRKRLLASAGELQKGNELALPSKYVTLLYHAKKYEKLKEK | |
| | EEDNEKKQEYVEQHRYEFDEIFEQISEFSKRYILADKNLDKILELFSNERDS | |
| | SISELAENFIHLFTFTSLGAPAAFKFFDKTIDRKRYTSTKEILDATLIHQSI | |
| | TGLYETRIDLSQLGGD | |
| CasEnd-282 | MKKKYSIGLDIGTNSVGWAVITDEYKVPSKKFKVLGNTDRQSIKKNLIGALL | 242 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLVDSTEKADLRLIYL | |
| | ALAHMIKFRGHFLIEGDLNSDNSDVQKLFIQLVQTYNQLFEENPINESGVDA | |
| | KAILSARLSKSRRLENLIAQFPNEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEEFYKFIKPILEKMDGTEELLAKLNREDLLRKQRTFDNGSIPHQIHL | |
| | GELHAILRRQEEFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMGKPEFLSGEQKKEIVDLLFKKNRKVTVKQLKEDYFKEI | |
| | ECFDSVEISGVEDRFNASLGTYHDLLKIIKDKDFLDNEENEDILEDIVLTLT | |
| | LFEDREMIEERLKKYANLFDKKVMKQLKRRRYTGWGRLSRKLINGIRDKQSG | |
| | KTILDFLKSDGFANRNFMQLIHDDSLTFKEAIKKAQVSGQGESLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTGQELDINR | |
| | LSDYDVDHIVPQSFLKDDSIDNKVLTRSEKARGKSDNVPSEEVVKKMKNYWR | |
| | QLLNAKLITQRKFDNLTKAERGGLSELDKAGFIKRQLVETRQITKHVAQILD | |
| | SRMNTKRDENDKLIRDVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEIKLANGEIRKRPVIETNEETGEIVWDKERDFATVRKVLSMPQV | |
| | NIVKKTEVQTGGFSKESILPKRESDKLIARKKDWDPKKYGGFDSPTVAYSVL | |
| | VVAKVEKGKTKKLKTVKELLGITIMERSSFEKDPVDFLEDKGYKEVKKDLII | |
| | KLPKYSLFELENGRKRMLASAGELQKGNELALPSKYVNFLYLASHYEKLKGS | |
| | PEDEEQKRLYVEQHKDYLDEIIDQISEFSERVILADKNLEKVLSAYNEFRDK | |
| | SINEQAENIIHLFTLTNLGAPAAFKYFDTTIDRKRYTSTKEVLNATLIHQSI | |
| | TGLYETRIDLSQLGGD | |
| CasEnd-283 | MDKKYSIGLDIGTNSVGWAVVTDDYKVPSKKFKVLGNTDRKSIKKNLLGALL | 243 |
| | FDSGETAEATRLKRTARRRYTRRKNRICYLQEIFSNEMAKVDESFFHRLEES | |
| | FLVEEDKRGERHPIFGNIVEEVAYHEKFPTIYHLRKKLADSPEKADLRLVYL | |
| | ALAHIIKFRGHFLIEGDLKSENTDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVSDES | |
| | TKAPLSASMVKRYEEHHKDLTLLKQFIREQLPEKYKEIFFDASKNGYAGYID | |
| | GGASQEEFYKYLKKILEKIDGSEEFLDKIEREDFLRKQRTFDNGSIPHQIHL | |
| | QELHAILRRQEEYYPFLKENKEKIEQILTFRIPYYVGPLARGNSRFAWLSRK | |
| | SDETITPWNFDEVVDKEASAEAFIERMTNFDKNLPNEKVLPKHSLLYEKFTV | |
| | YNELTKVKYVTEGGKKPEFFDANQKQEIFDLLFKKYRKVTKKQLKDFLFKEF | |
| | DEFRIVDISGVEDRFNASLGTYHDLLKIIKDKDFLDNEENEEILEDIILTLT | |
| | LFEDREMIKKRLEKYADLFDKKQLKKLERRRYTGWGRLSAKLINGIRDKQTG | |
| | KTILDYLIDDGFANRNFMQLIHDDNLSFKEEIAKAQVIGQTESLHETIANLA | |
| | GSPAIKKGILQSLKIVDELVKVMGRYEPENIVVEMARENQTTAKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLTEDDKAGFIKRQLVETRQITKHVAQILD | |
| | SRFNTEYDDNGKLIRDVKIITLKSKLVSQFRKDFELYKVREINDYHHAHDAY | |
| | LNAVVGTALIKKYPKLESEFVYGDYPKYNLRKMIAKSRKEIGKATAKMFFYS | |
| | NIMNFFKTDIKLADGTVRERPLIEVNEETGEIVWDKEKHIATVKKVLSMPQV | |
| | NIVKKTEVQTGGFSKESILPKRDSDKLIARKKDWDTSKYGGEDSPTVAYSVL | |
| | VIADVEKGKAKKLKTVKELVGITIMERSAFEKDPIAFLEDKGYQNIQKDNLI | |
| | KLPKYSLFELENGRKRLLASAGELQKGNELALPNHLVILLYHAKHIEKLKGS | |
| | PEDNEESLNYVEEHREEFDEILDQISEFSKRYILADANLEKLKELYEKNKEA | |
| | SIEELASSFINLLTFTALGAPAAFKFFGKTIDRKRYTSTKEILNATLIHQSI | |
| | TGLYETRIDLSKLGGD | |
| CasEnd-284 | MDKPYSIGLDIGTNSVGWAVVTDEYKVPSKKFKVLGNTDRKSIKKNLLGALL | 244 |
| | FDSGETAEATRLKRTARRRYTRRKNRILYLQEIFAEEMAKVDDNFFHRLEES | |
| | FLVEEDKRGERHPIFGNIVEEVAYHEKYPTIYHLRKHLADSPEKADLRLVYL | |
| | ALAHIIKFRGHFLIEGDLDAENTDVQELFQELLEVYDRTFEESHLQEEKVDA | |
| | EEILTEKISKSRRLENLLALFPGEKKNGLFGELLKLIVGLTPNFKSNFGLEE | |
| | DAKLQFSKDTYDEDLEELLGQIGDEYADLFVAAKKLYDAILLSGILTVKDSS | |
| | TKAPLSASMVQRYDEHHQDLTLLKKFIRKNLPEKYKEIFFDQSKNGYAGYID | |
| | GGASQEDFYKYLKKLLEKIDGSEYFLDKIEREDFLRKQRTFDNGSIPHQIHL | |
| | QELHAIIRRQEKYYPFLKENQEKIEKILTFRIPYYVGPLARGNSRFAWLSRK | |
| | SDETITPWNFDEVVDKEASAEAFIERMTNFDKNLPDEKVLPKHSLLYEKFTV | |
| | YNELTKVKYVTEGGKKPEFFDANMKQEIFDGVFKKYRKVTKKQLLDFLEKEF | |
| | DEFRIVEISGVEDRFNASLGTYHDLKKILGDKDFLDNPDNEEILEDIILTLT | |
| | LFEDREMIKKRLEKYEDLLDKEQIKKLERRRYTGWGRLSAKLINGIRDKQTG | |
| | KTILDYLIDDGYANRNFMQLIHDDSLSFKEEIAKAQVIGETESLHEVIANLA | |
| | GSPAIKKGILQSLKIVDELVKVMGRHEPENIVVEMARENQTTQKGQKNSRER | |
| | MKRLEESIKELGSEILKEHPVENTKLQNDKLYLYYLQNGRDMYTGEPLDIDN | |
| | LSDYDVDHIVPQSFLKDDSIDNRVLVSSAKARGKSDDVPSEEVVRKMKSFWK | |
| | KLLDAKLITQRKYDNLTKAERGGLTELDKAGFIKRQLVETRQITKHVAQILD | |
| | ERFNTEYDENGKLIRKVKIVTLKSKLVSQFRKEFELYKVREINNYHHAHDAY | |
| | LNAVVGKALIKKYPKLESEFVYGDYPVYDVYKFVAKSDREIGKATAKMFFYS | |
| | NIMNFFKSDVKLADGEIVERPDIEVNEETGEIAWDKDKHIATVKKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKDPVAFLESKGYQNIQKENII | |
| | KLPKYSLFELENGRKRLLASAGELQKGNELALPNHYVTLLYHAKHYEKLKGK | |
| | PEDIEKHLIYVEEHRDEFDELLDQISEFSKRYILADANLEKIKKLYEKNKEA | |
| | SIEELASSFINLLTFTALGAPAAFKFFGKNIDRKRYTSTKEILNATLIHQSI | |
| | TGLYETRIDLSKLGGD | |
| CasEnd-285 | MKKPYSIGLDIGTNSVGWAVVTDDYKVPSKKMKVLGNTDRSSIKKNLLGALL | 245 |
| | FDSGETAEATRLKRTARRRYTRRKNRILYLQEIFAEEMNKVDDSFFHRLEDS | |
| | FLVEEDKRGERHPIFGNIVEEVKYHEEFPTIYHLRKELADSPEKADLRLVYL | |
| | ALAHIIKFRGHFLIEGDLDTENTDVQQLFQEFLEVYDKTFEDSHLSEQNVQV | |
| | EEILTDKISKSAKKERVLKLFPNEKSNGFFAEFLKLIVGNQADFKKHENLEE | |
| | KAKLQFSKDTYEEDLETLLGQIGDEYADVFVAAKKLYDSILLSGILTVTDVS | |
| | TKAPLSASMVQRYEEHHEDLTKLKQFIRKKLPEKYKEFFFDTSKNGYAGYID | |
| | GGTSQEEFYKYLKKLLNKIAGSEYFLEKIEREDFLRKQRTFDNGSIPHQIHL | |
| | QELKAIIRRQAEYYPFLAENQDKIEKILTFRIPYYVGPLARGNSRFAWASRK | |
| | SDETITPWNFDEIIDKEKSAEAFINRMTNNDLYLPEEKVLPKHSLLYEKFTV | |
| | YNELTKVKYITEQMGETKFFDANMKQEIFDGVFKKYRKVTKKKLLNFLDKEF | |
| | DEFRIVDLSGVEKAFNASLGTYHDLKKILGDKEFLDDPDNEDILEDIIQTLT | |
| | LFEDREMIRKRLSKYSDLFTKEQLKKLERRHYTGWGRLSAKLINGIRDKETR | |
| | KTILDYLIDDGRSNRNFMQLIHDDGLSFKEEIAKAQVIGETDSLHQVVADLA | |
| | GSPAIKKGILQSLKIVDELVKVMGRYNPENIVVEMARENQTTNKGQRNSRER | |
| | LKGLTDAIKNLGSKILKEYPVDNQQLQNDRLYLYYLQNGKDMYTGEELDIDN | |
| | LSQYDVDHIIPQSFIKDDSIDNRVLVSSAKARGKSDDVPSIEVVRKMKSFWS | |
| | KLLDAKLISQRKFDNLTKAERGGLTEDDKAGFIKRQLVETRQITKHVAQILD | |
| | ERFNTEKDENGKLIRKVKIVTLKSKLVSNFRKEFELYKVREINDYHHAHDAY | |
| | LNAVVAKALIKKYPKLEPEFVYGDYPKYNSYKLVGETKNERGKATAKMFFYS | |
| | NIMNFFKSDVKLADGTEVERPMIEVNEETGEIIWDKKKHISIVKKVLSMPQV | |
| | NIVKKTEVQTGGFSKESILPKRNSDKLIARKKKWDTKKYGGFDSPIVAYSVL | |
| | VIADIEKGKAQKLKTVKELVGITIMERSRFEKDPVAFLENKGYQNIREENII | |
| | KLPKYSLFELENGRKRLLASAGELQKGNELALPNHLVTLLYHAKNIEKLDEK | |
| | EEEKPKHKNYVEKHRSEFKELLDQVSEFSKRYILADKNLEKIEELYAQNEEA | |
| | SIEELASSFINLLTFTALGAPADFKFFGKNIDRKRYTSTTEILNATLIHQSI | |
| | TGLYETRIDLSKLGED | |
| CasEnd-286 | MDKPYSIGLDIGTNSVGWAVVTDEYKVPSKKFKVLGNTDRHSIKKNLLGALL | 246 |
| | FDSGETAEATRLKRTARRRYTRRKNRIRYLQEIFAEEMNKVDDSFFHRLEES | |
| | FLVEEDKRGERHPIFGNIVEEVAYHEEYPTIYHLRKKLADSTEKADLRLVYL | |
| | ALAHIIKFRGHFLIEGDLNSENTDVQKLFKEFVQTYDNTFEESHLQEETVDA | |
| | KSILTAKISKSRRLENLIKQFPGEKKNGLFGNLIALSLGLQPNFKINFELSE | |
| | DAKLQFSKDTYDEDLENLLAQIGDEYADLFVAAKNLSDAILLSGILTVNTEI | |
| | TKAPLSASMIKRYDEHHQDLTLLKKFIRQQLPEKYKEIFFDASKNGYAGYID | |
| | GGASQEEFYKYIKPILSKIDGSEYFLDKIEREDFLRKQRTFDNGSIPHQIHL | |
| | QELHAILRRQEEFYPFLKENQEKIEKILTFRIPYYVGPLARGNSRFAWASRK | |
| | SDETITPWNFEEVVDKEASAQAFIERMTNNDKNLPEEKVLPKHSLLYEKFTV | |
| | YNELTKVKYVTEGMRKPAFFDSEQKQEIVDLLFKTNRKVTKKKLKEYLFKEI | |
| | ECFDSVEISGVEDRFNASLGTYHDLLKIIKDKDFLDNEENEEILEDIVLTLT | |
| | LFEDREMIKERLSKYADLFDKKVLKKLERRRYTGWGRLSAKLINGIRDKQSG | |
| | KTILDYLIDDGFANRNFMQLIHDDSLSFKEEIKKAQVIGDTDSLHEVIANLA | |
| | GSPAIKKGILQSIKIVDELVKVMGRHNPENIVIEMARENQTTQKGQRNSRER | |
| | LKRLEEAIKELGSNILKEHPVENTQLQNDKLYLYYLQNGKDMYTGEELDIDR | |
| | LSDYDVDHIIPQSFIKDDSIDNRVLTSSAKARGKSDNVPSEEVVKKMKSFWR | |
| | QLLDSKLISQRKFDNLTKAERGGLTELDKAGFIKRQLVETRQITKHVAQILD | |
| | SRFNTKYDENDKLIREVKIITLKSKLVSDFRKDFQLYKVREINDYHHAHDAY | |
| | LNAVVGTALIKKYPKLESEFVYGDYKVYNSYKMIAKSEQEIGKATAKYFFYS | |
| | NIMNFFKTDITLANGEIRKRPLIETNEETGEIVWDKDKDFATVRKVLSMPQV | |
| | NIVKKTEVQTGGFSKESILPKRNSDKLIARKKDWDTKKYGGFDSPTVAYSVL | |
| | VIADIEKGKAKKLKTVKELVGITIMERSAFEKNPVAFLEDKGYQNIQKENII | |
| | KLPKYSLFELENGRKRLLASAGELQKGNELALPNKYVTLLYHAKHYEKLKES | |
| | PEDNPEKLEYVEQHRDEFDEIFDQISEFSERYILADKNLEKIQEAYAKNEDA | |
| | SIEELAESFINLLTFTALGAPAAFKFFGKTIDRKRYTSTKEILNATLIHQSI | |
| | TGLYETRIDLSQLGGD | |
| CasEnd-287 | MDKPYSIGLDIGTNSVGWAVVTDEYKVPSKKFKVLGNTDRHSIKKNLIGALL | 247 |
| | FDSGETAEATRLKRTARRRYTRRKNRIRYLQEIFAEEMSKVDDSFFHRLEES | |
| | FLVEEDKRGERHPIFGNIVDEVAYHEKYPTIYHLRKHLADSSEKADLRLIYL | |
| | ALAHIIKFRGHFLIEGDLNAENTDVQKLFKQLVDVYDQTFEESHLSEETVDA | |
| | KSILTEKVSKSRRLENLIKCFPNEKRNGLFGNLIALSLGLTPNFKSNFELAE | |
| | DAKLQFSKDTYDEDLENLLAQIGDQYADLFVAAKNLYDAILLSGILTVNTEI | |
| | TKAPLSASMIKRYDEHHQDLTLLKHFVRTQLPEKYKEIFFDVSKNGYAGYID | |
| | GGASQEEFYKYLKPILSKIDGTEYLLDKIEREDFLRKQRTEDNGSIPHQIHL | |
| | QELKAILRRQEDYYPFLKENQEKIEKILTFRIPYYVGPLARGNSRFAWMSRK | |
| | SDETITPWNFEEVIDKEASAQAFIERMTNFDKNLPEEKVLPKHSLLYEKFTV | |
| | YNELTKVKYVTEGMRKPAFFSANQKQEIVDLLFKKNRKVTKKKLKNFLFKKF | |
| | DCFDIVEISGVEDRFNASLGTYHDLLKIIKDKDFLDNSENEEIFEEIILTLT | |
| | LFEDREMIEERLKKYAHLFDKKVLKKLKRRRYTGWGRLSKKLINGIRDKQSG | |
| | KTILDYLIDDGFSNRNFMQLIHDDSLTFKEEIAKAQVIGQSESLHETIANLA | |
| | GSPAIKKGILQSLKIVDELVKVMGRHNPENIVIEMARENQTTQKGQRNSRER | |
| | MKRLEEAIKELGSQILKEHPVENTQLQNDKLYLYYLQNGKDMYTGEELDIDR | |
| | LSDYDVDHIIPQSFIKDDSIDNRVLTSSAEARGKSDNVPSEEVVKKMKSFWR | |
| | RLLDSKLISQRKFDNLTKAERGGLSELDKAGFIKRQLVETRQITKHVAQILD | |
| | SRFNTKYDENDKLIRNVKIITLKSKLVSDFRKDFQFYKVREINDYHHAHDAY | |
| | LNAVVGTALIKKYPKLESEFVYGDYKVYDSRKMIAKSEQEIGKATAKYFFYS | |
| | NIMNFFKSEITLANGEIRKRPLIETNEETGEIVWDKKKDIATVRKVLSMPQV | |
| | NIVKKTEVQTGGFSKESILPKRNSDKLIARKKDWDTKKYGGFDSPTVAYSVL | |
| | VVADIEKGKSKKLKTVKELVGITIMERSAFEKDPIAFLEKKGYKNIQKDNII | |
| | KLPKYSLFELENGRKRLLASAGELQKGNELALPNHYVTLLYHAKRYEKLKGS | |
| | PEDNEKHLEYVEQHRAEFDEILSQISEFSERYILADKNLEKIQELYAKNRDE | |
| | DIKELASSFINLFTFTALGAPAAFKFFDKTIDRKRYTSTTEVLNATLIHQSI | |
| | TGLYETRIDLSQLGGD | |
| CasEnd-288 | MDKKYSIGLDIGTNSVGWAVITDEYKVPSKKFKVLGNTDRHSIKKNLIGALL | 248 |
| | FDSGETAEATRLKRTARRRYTRRKNRICYLQEIFSNEMAKVDESFFHRLEES | |
| | FLVEEDKRGERHPIFGNIVDEVAYHEKYPTIYHLRKHLADSTEKADLRLIYL | |
| | ALAHMIKFRGHFLIEGDLNSENTDVQKLFKQFVEAYDQTFEESHLEEITVDA | |
| | KAILTEKLSKSRRLENLIAQFPGEKKNGLFGNLIALSLGLTPNFKSNFKLSE | |
| | DAKLQFSKDTYDEDLENLLGQIGDEYADLFVAAKNLYDAILLSGILTVNDES | |
| | TKAPLSASMIKRYDEHHQDLTLLKAFVREQLPEKYKEIFFDESKNGYAGYID | |
| | GGASQEEFYKYIKPILEKVDGSEYFLDKIEREDFLRKQRTFDNGSIPHQIHL | |
| | QELHAILRRQEEYYPFLKENQEKIEKILTFRIPYYVGPLARGNSRFAWMTRK | |
| | SDETITPWNFEEVVDKEASAQAFIERMTNNDKNLPEEKVLPKHSLLYEKFTV | |
| | YNELTKVKYVTEGMGKAEFFDANQKQEIFDGLFKKNRKVTKKKLLDFLFKEF | |
| | EEFRIVDISGVEDAFNASLGTYHDLLKIIKDKEFLDNEENEDILEDIVLTLT | |
| | LFEDREMIEKRLKKYADLFDKKVLKKLERRRYTGWGRLSAKLINGIRDKQSG | |
| | KTILDYLIDDGFANRNFMQLIHDDSLTFKEEIKKAQVIGESDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRKSDKLIARKKDWDTKKYGGFDSPTVAYSVL | |
| | VVADVEKGKAKKLKTVKELVGITIMERSAFEKNPVAFLEDKGYQNIQEDNII | |
| | KLPKYSLFELENGRKRLLASAGELQKGNELALPNKYVTLLYHAKNYEKLKGK | |
| | SEDEEEHLEYVSKHNDEFKEILDQISEFSERYILADKNLEKIKELYEQNEDY | |
| | SISELASSFINLLTFTALGAPAAFKFFGTTIDRKRYTSTKEILNSTLIHQSI | |
| | TGLYETRIDLSKLGGD | |
| CasEnd-289 | MDKKYSIGLDIGTNSVGWAVITDEYKVPSKKFKVLGNTDRHSIKKNLIGALL | 249 |
| | FDSGETAEATRLKRTARRRYTRRKNRIRYLQEIFSEEMSKVDDSFFHRLEES | |
| | FLVEEDKRHERHPIFGNIVDEVAYHENYPTIYHLRKKLADSTEKADLRLIYL | |
| | ALAHIIKFRGHFLIEGDLNPDNTDVDKLFIQLVQTYNQLFEENPIHEENVDA | |
| | KAILTAKLSKSRRLENLIAQIPGEKKNGLFGNLIALSLGLTPNFKSNEDLAE | |
| | DAKLQLSKDTYDEDLDNLLAQIGDQYADLFLAAKNLSDAILLSDILTVNDEI | |
| | TKAPLSASMIKRYDEHHQDLTLLKALVREQLPEKYKEIFFDQSKNGYAGYID | |
| | GGASQEEFYKYIKPILSKLDGTEELLAKLNREDLLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPNEKVLPKHSLLYEYFTV | |
| | YNELTKVKYVTEGMRKPEFLSAEQKEEIVDLLFKTNRKVTVKQLKEFLFKKI | |
| | DCFDSVEISGVEDRFNASLGTYHDLLKIIKDKDFLDNEENEAILEDIVLTLT | |
| | LFEDKEMIEERLSKYAHLFDKKVLKQLKRRRYTGWGRLSRKLINGIKDKQSG | |
| | KTILDFLKSDGFANRNFMQLIHDDSLTFKEDIQKAQVEGQGESLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIREVKIITLKSKLVSDFRKEFQFYKVREINDYHHAHDAY | |
| | LNAVVGKALIKKYPKLESEFVYGDYKVYDVRKMIAKSEQEIGKATAKYFFYS | |
| | NIMNFFKSEITLANGEIRKRPLIETNEETGEIVWDKGKDFATVKKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLEAKGYKNVQKHLII | |
| | KLPKYSLFELENGRKRLLASAGELQKGNELALPSKYVTLLYHASHYEKLKGK | |
| | PEDEEKKLEYVEQHRYYFDEILEQIVEFSKRYILADKNLEKIQELYSENESY | |
| | PIEELAENFIHLFTFTALGAPAAFKFFDTDIDRKRYTSTTEILNATLIHQSI | |
| | TGLYETRIDLSQLGGD | |
| CasEnd-290 | MDKPYSIGLDIGTNSVGWAVVTDEYKVPSKKFKVLGNTDRHSIKKNLLGALL | 250 |
| | FDSGETAEATRLKRTARRRYTRRKNRIRYLQEIFAEEMNKVDDSFFHRLEES | |
| | FLVEEDKRGERHPIFGNIVDEVAYHEKYPTIYHLRKKLADSTEKADLRLVYL | |
| | ALAHMIKFRGHFLIEGDLDAENTDVQKLFEEFVQVYDNTFEESHLSEETVDA | |
| | SSILTAKLSKSRRLENLIKLYPNEKKNGLFGNLIALSLGLQPNFKTNFNLAE | |
| | DAKLQFSKDTYEEDLENLLGQIGDEYADLFVAAKNLYDAILLSGILTVNTEI | |
| | TKAPLSASMIKRYDEHHQDLTLLKEFIRANLPEKYKEIFFDETKNGYAGYID | |
| | GGAKQEEFYKYLKPILSKIDGSEYFLDKIEREDFLRKQRTFDNGSIPHQIHL | |
| | QELHAILRRQGEFYPFLKENQEKIEKILTFRIPYYVGPLARGNSRFAWASRK | |
| | SDETITPWNFDEVVDKESSAEAFIERMTNNDKNLPEEKVLPKHSLLYEKFTV | |
| | YNELTKVKYITEQMRKPAFFDANVKKEIFDLVFKKNRKVTKKKLLDYLFKEF | |
| | DEFRIVDISGVEKSFNASLGTYHDLLKIIKDKEFLDNEENEKILEDIVLTLT | |
| | LFEDREMIDKRLEKYADLFDKKVLKKLERRRYTGWGRLSAKLINGIRDKQSG | |
| | KTILDYLIDDGFANRNFMQLIHDDSLSFKEEIQKAQVIGQTDSLHEVIADLA | |
| | GSPAIKKGILQSLKIVDELVKVMGRHKPENIVIEMARENQTTQKGQRNSRER | |
| | LKRLEEAIKELGSKILKEHPVENTQLQNDKLYLYYLQNGKDMYTGEELDIDR | |
| | LSDYDVDHIIPQSFIKDDSIDNRVLTSSKEARGKSDDVPSEEVVKKMKSFWN | |
| | RLLKAKLISQRKFDNLTKAERGGLTELDKAGFIKRQLVETRQITKHVAQILD | |
| | SRFNTKYDENDKLIRKVKIVTLKSKLVSQFRKEFQLYKVREINDYHHAHDAY | |
| | LNAVVGKALIKKYPKLESEFVYGDYKVYNSRKMIAKSEQEIGKATAKYFFYS | |
| | NIMNFFKTDVTLANGEIRKRPLIETNKETGEIVWDKEKDFATVRKVLSMPQV | |
| | NIVKKTEVQTGGFSKESILPKRDSDKLIARKKDWDTKKYGGFDSPTVAYSVL | |
| | VVADIEKGKVKKLKTVKELVGITIMERSSFEKNPIAFLEDKGYKNVQKENII | |
| | KLPKYSLFELENGRKRLLASAGELQKGNELALPQKYVTLLYHAKHYEKLKES | |
| | PEDNEKHLLYVEQHRSYFDEILDQISEFSKRYILADKNLDKIKELYAENEGA | |
| | DVEELASSFINLLTFTALGAPAAFKFFDADIDRKRYTSTTEILDATLIHQSI | |
| | TGLYETRIDLSQLGGD | |
| CasEnd-291 | MKKDYTIGLDIGTNSVGYAVVYAEYKVVSKKFKVLGNGQRKSIKKNFWGVRL | 251 |
| | FDSGETAEATRLKRTARRRYTRRKNRIRYLQEIFNEEMNAVDQNFFHRLEES | |
| | FLVEEDKRNERHPIFATIVEEVAYHEEYPTIYHLRKHLADCKEQSDIRLVYL | |
| | ALAHIIKFRGHFLIEGKLSTENTSIRENFKKFLQIYNQTFSVQEDGSETSGV | |
| | EELLQEKASRQKKAENVLKLFPTEKANGTFMQFLKLIVGNQGNFKKTENLSE | |
| | DVKLQFSKDTYEEQLEELLANVGDDYAEVFVAAKNVYDAIELSGILTVKDFT | |
| | TKAKLSASMVKRYDEHHQDLTKLKKFIRDKLPEKYKDIFFNEKKNGYAGYID | |
| | GGAKQDDFYKYLKKVLNRAEGADYFLDKIDKEEFLRKQRTFDNGSIPHQIHL | |
| | EELRAIIGKQAKYYPFLAENKAKIEQILTFRIPYYVGPLARGNSRFAWLSRK | |
| | KQETITPWNYGELIDEGKTATDFIERMTNYDKNLPQEKVLPKHSMLYEKFTV | |
| | FNELTKVKYIDDRMGETQFFSSLEKREIFEELFKKSRKVKLTDLENFLKNQF | |
| | YMIEVSKISGVEKSFNASYGTYHDFRKIGIEREVLDAPENEEMFEEIIKILT | |
| | VFEDRKMIREQLSKYGDFFEPKILKKLERRRYTGWGRLSAKLINGIKDKHTK | |
| | KTILDYLMRDDAKNRNFMQLIHDDSLSFKEEIAKEQADEQTDSLHEIIANLA | |
| | GSPAIKKGILQSLKIVDEIVKVMGRYAPKNIVVEMARENQTTQKGQDNSRER | |
| | LKNLEDAIKELGSNILKEYPLDNTDLQRDKLYLYYLQNGKDMYTGLDLDIDQ | |
| | LSDYDVDHIIPQSFIKDDSIDNLVLVSSSKARGKSDDVPSIEIVEKMKPEWE | |
| | RLKNANLISQRKYDNLTKAERGGLTELDKAGFIKRQLVETRQITKHVAQILH | |
| | QRFNSEKTSEGKLERRTKIITLKSKLTSQFRKIYGLYKVREINDYHHGHDAY | |
| | LNGVVANALIKVYPNLESEFVYGDYRVFNSFKLVRETDEKIGKATAKKEFYS | |
| | NLMRFFKSDQKLADDSVIEKPRVEVDDENGEILWGQKKDISTVKKVMSMPQV | |
| | NIVKKTEVQTGGFSKESILPKRDSDKLIARKKDWDTRKYGGFDSPTVAYSVV | |
| | ISYEKGKKKKQKIKTVKDIVGITIMERSKFEENEVQFLIDKGFVNPKEIVEV | |
| | KLPKYTLYEVENGRKRLLASAGELQKGNELALPNHYVTLLYHAKHYEKIKEK | |
| | EKEEKNSYNYLVDHRKEFDELFEQVKEFAERYTLADKNLEKITTLFEENHEA | |
| | DIKLIAQSFLNLMQFNAMGAPAAFKFFGQVIDRKRYTSIKELLNATIIHQSI | |
| | TGLYETRIKLGKLGEE | |
| CasEnd-292 | MDKPYSIGLDIGTNSVGWAVITDEYKVPSKKFKVLGNTDRHSIKKNLLGALL | 252 |
| | FDSGETAEATRLKRTARRRYTRRKNRIRYLQEIFAEEMSKVDDSFFHRLEES | |
| | FLVEEDKRGERHPIFGNIVEEVAYHEEYPTIYHLRKHLADSTEKADLRLVYL | |
| | ALAHMIKFRGHFLIEGDLDSENTDVQKLFEQFVQVYDNTFEESHLSEETVDA | |
| | ESILTAKISKSRRLENLIKLFPGEKKNGLFGNLIALILGLQPNFKTNFELSE | |
| | DAKLQFSKDTYEEDLENLLGQIGDDYADLFVAAKNLYDAILLSGILTVNTEI | |
| | TKAPLSASMIKRYDEHHQDLTLLKKFIRENLPEKYKEIFFDESKNGYAGYID | |
| | GGASQEEFYKYIKNLLSKIDGSEYFLDKIEREDFLRKQRTFDNGSIPHQIHL | |
| | QELHAILRRQGEYYPFLKENQEKIEKILTFRIPYYVGPLARGNSRFAWASRK | |
| | SDETITPWNFEEVVDKESSAEAFIERMTNNDKNLPEEKVLPKHSLLYEKFTV | |
| | YNELTKVKYITEGMRKPAFFDANQKQEIFDGLFKKNRKVTKKKLLEFLFKEF | |
| | DEFRIVDISGVEKAFNASLGTYHDLLKIIKDKDELDNEENEDILEDIVLTLT | |
| | LFEDREMIEKRLSKYADLFDKKVLKKLERRRYTGWGRLSAKLINGIRDKQSG | |
| | KTILDYLIDDGFANRNFMQLIHDDSLSFKEEIQKAQVIGQTDSLHETIADLA | |
| | GSPAIKKGILQSLKIVDELVKVMGRHAPENIVIEMARENQTTQKGQRNSRER | |
| | LKRLEESIKELGSKILKEHPVDNTQLQNDKLYLYYLQNGRDMYTGEELDIDR | |
| | LSDYDVDHIIPQSFIKDDSIDNRVLTSSAKARGKSDDVPSEEVVKKMKSFWQ | |
| | QLLDSKLISQRKFDNLTKAERGGLTELDKAGFIKRQLVETRQITKHVAQILD | |
| | SRFNTKYDENDKLIRDVKIITLKSKLVSQFRKEFGFYKVREINDYHHAHDAY | |
| | LNAVVGKALIKKYPKLESEFVYGDYPVYNSYKMIAKSEQEIGKATAKYFFYS | |
| | NIMNFFKKDVTLANGEIRKRPLIETNEETGEIVWDKEKHIATVKKVLSMPQV | |
| | NIVKKTEVQTGGFSKESILPKRNSDKLIARKKDWDTKKYGGFDSPTVAYSVL | |
| | VVADIEKGKAKKLKTVKELVGITIMERSSFEKNPVAFLENKGYQNIQKENII | |
| | KLPKYSLFELENGRKRLLASAGELQKGNELALPNKYVTLLYHAKHYEKLKES | |
| | PEDNPEHLEYVEEHRDEFDELFDQISEFSKRYILADKNLEKIKELYNENEEA | |
| | SIEELAESFINLLTFTALGAPAAFKFFGVDIDRKRYTSTTEILNATLIHQSI | |
| | TGLYETRIDLSQLGGD | |
| CasEnd-293 | MDKKYSIGLDIGTNSVGWAVITDEYKVPSKKFKVLGNTDRHSIKKNLIGALL | 253 |
| | FDSGETAEATRLKRTARRRYTRRKNRICYLQEIFSNEMAKVDDSFFHRLEES | |
| | FLVEEDKRGERHPIFGNIVEEVAYHEKYPTIYHLRKHLADSDEKADLRLVYL | |
| | ALAHIIKFRGHFLIEGDLKSENSDVQKLFKDLVEVYDQTFEESHLSEETVDA | |
| | ESILTEKISKSRRLENLIKQFPNEKKNGLFGNLIALSLGLQPNFKTNEDLSE | |
| | DAKLQFSKDTYDEDLENLLGQIGDEYADLFLAAKNLYDAILLSGILTVNDES | |
| | TKAPLSASMIKRYDEHHQDLTLLKKFIREQLPEKYKEIFFDETKNGYAGYID | |
| | GGASQEEFYKYIKKILEKVDGSEYFLEKIEREDFLRKQRTFDNGSIPHQIHL | |
| | QELHAILRRQEEYYPFLKENQEKIEKILTFRIPYYVGPLARGNSRFAWMSRK | |
| | SDETITPWNFDEVVDKEASAEAFIERMTNFDKNLPEEKVLPKHSLLYEKFTV | |
| | YNELTKVKYVTEGMGKPKFFDANMKQEIFDGLFKKNRKVTKKKLLDFLDKEF | |
| | DEFRIVDISGVEKAFNASLGTYHDLLKIIKDKDFLDNEENEDILEDIVLTLT | |
| | LFEDREMIEKRLEKYADLFDKKQLKKLERRRYTGWGRLSAKLINGIRDKQSG | |
| | KTILDYLIDDGFANRNFMQLIHDDALSFKEEIQKAQVIGEGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDTKKYGGFDSPTVAYSVL | |
| | VVADIEKGKKKKLKTVKELVGITIMERSRFEKNPVAFLEDKGYKNIQEEKII | |
| | KLPKYSLFELENGRKRLLASAGELQKGNELALPAHYVTLLYHAKNYEKLKEK | |
| | PEDEEKHLEYVDKHRDEFKEILDQISEFSERYILADGNLEKIKELYKKNEDA | |
| | SISELASSFINLLTFTALGAPAAFKFLGSTIDRKRYTSTTEILNATLIHQSI | |
| | TGLYETRIDLSKLGGD | |
| CasEnd-294 | MKKPYSIGLDIGTNSVGWAVVTDDYKVPSKKMKVLGNTDRQSIKKNLLGALL | 254 |
| | FDSGETAEATRLKRTARRRYTRRKNRILYLQEIFAEEMNKVDDSFFHRLEDS | |
| | FLVEEDKRGERHPIFGNIVEEVAYHEKFPTIYHLRKHLADSTEKADLRLVYL | |
| | ALAHIIKFRGHFLIEGDLNTENTDVQKLFKKFVEVYDRTFEESHLSEETVDA | |
| | EEILTEKVSKSRKLENLLKQFPNEKKNGLFGNLIALSLGLQPNFKINFELSE | |
| | DAKLQFSKDTYDEDLENLLGQIGDEYADLFVAAKNLYDAILLSGILTVNDLS | |
| | TKAPLSASMIKRYEEHHEDLILLKKFIRKNLPEKYKEIFFDESKNGYAGYID | |
| | GGTSQEEFYKYIKNLLSKIDGSEYFLDKIEREDFLRKQRTFDNGSIPHQIHL | |
| | QELKAILRRQGEFYPFLKENQEKIEKILTFRIPYYVGPLARGNSRFAWASRK | |
| | SDETITPWNFDEVIDKEKSAEAFIERMTNNDLYLPEEKVLPKHSLLYEKFTV | |
| | YNELTKVKYVTEGMGEAEFFDANLKQEIFDGLFKKERKVTKKKLLEFLDKEF | |
| | DEFRIVDISGVEKAFNASLGTYHDLLKIIKDKEFLDNEENEDILEDIVLTLT | |
| | LFEDREMIKQRLSKYADLFDKKVLKKLERRHYTGWGRLSAKLINGIRDKQSG | |
| | KTILDYLIDDGFANRNFMQLIHDDNLSFKEEIAKAQVIGESDSLHEVVAELA | |
| | GSPAIKKGILQSLKIVDELVKVMGRYAPENIVVEMARENQTTAKGQRNSRER | |
| | LKRLEEAIKELGSKILKEHPVENQQLQNDRLYLYYLQNGKDMYTGEELDIDR | |
| | LSQYDVDHIIPQSFIKDDSIDNRVLTSSAKARGKSDDVPSEEVVKKMKSEWS | |
| | KLLNAKLISQRKFDNLTKAERGGLTEDDKAGFIKRQLVETRQITKHVAQILD | |
| | ERFNTEFDENNKLIRKVKIITLKSKLVSNFRKEFEFYKVREINDYHHAHDAY | |
| | LNAVVGKALIKKYPKLEPEFVYGDYPKYNSYKLVAKSEQERGKATAKMFFYS | |
| | NIMNFFKSDIKLADGTIVERPMIEVNEETGEIAWDKTKHIATVKKVLSMPQV | |
| | NIVKKTEVQTGGFSKESILPKRNSDKLIARKKKWDTKKYGGFDSPTVAYSVL | |
| | VVADIEKGKSKKLKTVKELVGITIMERSRFEKNPVAFLEDKGYQNIQEENII | |
| | KLPKYSLFELENGRKRLLASAGELQKGNELALPNKLVTLLYHAKHIEKLDEK | |
| | PEDIEKHLEYVEKHRDEFKEILDQISEFSKRYILADKNLEKIEELYAKNEDA | |
| | SIEELASSFINLLTFTALGAPADFKFFGKTIDRKRYTSTTEILNATLIHQSI | |
| | TGLYETRIDLSKLGGD | |
| CasEnd-295 | MDKPYSIGLDIGTNSVGWAVVTDEYKVPSKKFKVLGNTDRHSIKKNLLGALL | 255 |
| | FDSGETAEATRLKRTARRRYTRRKNRILYLQEIFAEEMNKVDDSFFHRLEES | |
| | FLVEEDKRNERHPIFGNIVDEVAYHENYPTIYHLRKKLADSPEKADLRLIYL | |
| | ALAHIIKFRGHFLIEGDLNVENTDVQKLFKDFVETYDQTFEESHLSEISVDA | |
| | KEILTAKISKSRKLENLIKQFPNEKKNGLFGNLIKLSLGLQPNFKSNFKLSE | |
| | DAKLQFSKDTYEEDLENLLAQIGDEYADLFLAAKNLYDAILLSGILTVNTEI | |
| | TKAPLSASMVKRYDEHHQDLTLLKKFIREQLPEKYKEIFFDKSKNGYAGYID | |
| | GGASQEEFYKYIKKILSKIEGSEYFLDKIEREDFLRKQRTFDNGSIPHQIHL | |
| | EELKAILRRQEEYYPFLKENREKIEKILTFRIPYYVGPLARGNSRFAWASRK | |
| | SDETITPWNFEEVVDKEKSAEDFIERMTNNDKNLPEEKVLPKHSLLYEKFTV | |
| | YNELTKVKYVTEGMRKPAFFDAGQKKEIVDLLFKTNRKVTKKKLLEFLFKEF | |
| | DEFDIVDISGVEKAFNASLGTYHDLLKIIKDKDFLDNEENEKILEDIILTLT | |
| | LFEDREMIKKRLSKYANLFDKKVLKKLERRRYTGWGRLSAKLINGIRDKQSG | |
| | KTILDYLIDDGFSNRNFMQLIHDDSLSFKEEIQKAQVIGQTDSLHQTIADLA | |
| | GSPAIKKGILQSIKIVDELVKVMGRHAPENIVIEMARENQTTQKGQRNSRER | |
| | LKKLEESIKELGSQILKEHPVDNTQLQNDKLYLYYLQNGKDMYTGEELDIDR | |
| | LSDYDVDHIIPQSFIKDDSIDNRVLTSSAKARGKSDDVPSEEVVKKMKSFWR | |
| | QLLDAKLISQRKFDNLTKAERGGLTELDKAGFIKRQLVETRQITKHVAQILD | |
| | SRFNTKYDENDKLIRDVKIITLKSKLVSQFRKEFGFYKVREINDYHHAHDAY | |
| | LNAVVGKALIKKYPKLESEFVYGDYKVYNSYKMIAKSEQEIGKATAKYFFYS | |
| | NIMNFFKEEITLANGEIRKRPLIETNEETGEIVWDKDKHIATVKKVLSMPQV | |
| | NIVKKTEVQTGGFSKESILPKRESDKLIARKKDWDTKKYGGFDSPTVAYSVL | |
| | VVADIEKGKAKKLKTVKELVGITIMERSSFEKNPVAFLEKKGYQNIQKENII | |
| | KLPKYSLFELENGRKRLLASAGELQKGNELALPNKYVTLLYHAKNYEKLKES | |
| | PEDNEKHLEYVEEHRDEFDEIFDQISEFSKRYILADKNLEKILELYDENRDA | |
| | PIKELAESFINLLTFTALGAPAAFKFFDKTIDRKRYTSTTEILDATLIHQSI | |
| | TGLYETRIDLSQLGGD | |
| CasEnd-296 | MDKPYSIGLDIGTNSVGWAVITDEYKVPSKKFKVLGNTDRHSIKKNLLGALL | 256 |
| | FDSGETAEATRLKRTARRRYTRRKNRIRYLQEIFAEEMSKVDDSFFHRLEES | |
| | FLVEEDKRGERHPIFGNIVDEVAYHEKYPTIYHLRKHLADSPEKADLRLVYL | |
| | ALAHMIKFRGHFLIEGDLKAENTDVQKLFINFVETYDNTFEESHLSEITVDA | |
| | SSILTEKVSKSRRLENLIKQFPTEKKNGLFGNLIALSLGLQPNFKSNFELSE | |
| | DAKLQFSKDTYEEDLENLLAQIGDQYADLFVAAKNLYDAILLSGILTVKTEI | |
| | TKAPLSASMIKRYDEHHQDLTLLKALIRENLPEKYKEIFFDESKNGYAGYID | |
| | GGASQEEFYKYIKPILLKMEGSEYFLDKIEREDFLRKQRTFDNGSIPHQIHL | |
| | QELHAILRRQEEYYPFLKENQEKIEKILTFRIPYYVGPLARGNSRFAWASRK | |
| | SDETITPWNFEEVVDKEASAEAFIERMTNNDKNLPEEKVLPKHSLLYEKFTV | |
| | YNELTKVKYVTERMRKPAFFDAEMKQEIVDLLFKENRKVTVKQLLEYLFKEF | |
| | DEFRSVDISGVEDRFNASLGTYHDLLKIIKDKAFLDNEENEDILEDIILTLT | |
| | LFEDREMIKKRLSKYADLFDKKVLKKLERRRYTGWGRLSAKLINGIRDKQTG | |
| | KTILDYLIDDGFANRNFMQLIHDDSLSFKEEIAKAQVIGQGDSLHETIADLA | |
| | GSPAIKKGILQSIKIVDELVKVMGRHAPENIVIEMARENQTTQKGQRNSRER | |
| | LKRLEESIKKLGSKILKEHPVENTQLQNDKLYLYYLQNGKDMYTGEELDIDN | |
| | LSDYDVDHIIPQSFIKDDSIDNRVLTSSAKARGKSDDVPSEEVVKKMKSFWR | |
| | QLLKAKLISQRKFDNLTKAERGGLTELDKAGFIKRQLVETRQITKHVAQILD | |
| | SRFNTKYDENDKLIRKVKIITLKSKLVSQFRKDFGFYKVREINDYHHAHDAY | |
| | LNAVVGTALIKKYPKLESEFVYGDYPVYNSRKMIAKSEQEIGKATAKYFFYS | |
| | NIMNFFKSDVTLANGEIRKRPLIETNEETGEIAWDKEKDFATVRKVLSMPQV | |
| | NIVKKTEVQTGGFSKESILPKRDSDKLIARKKDWDTKKYGGFDSPTVAYSVL | |
| | VVADIEKGKSKKLKTVKELVGITIMERSAFEKNPIAFLEKKGYQNIQKDNII | |
| | KLPKYSLFELENGRKRLLASAGELQKGNELALPNKYVTLLYHAARYEKSKES | |
| | PEDNPNHLLYVEKHKEEFDEILDQISEFSKRYILADSNLEKIEELYANNNKK | |
| | DISELASSFINLFTFTALGAPAAFKFFGATIDRKRYTSTTEILNATLIHQSI | |
| | TGLYETRIDLSQLGGD | |
| CasEnd-297 | MKKPYSIGLDIGTNSVGWAVVTDEYKVPSKKFKVLGNTDRQSIKKNLLGALL | 257 |
| | FDSGETAEATRLKRTARRRYTRRKNRILYLQEIFAEEMNKVDDSFFHRLEES | |
| | FLVEEDKRGERHPIFGNIVEEVAYHEKYPTIYHLRKHLADSTEKADLRLVYL | |
| | ALAHIIKFRGHFLIEGDLNTENTDVQKLFKQFLEVYDQTFEESHLSEETVDA | |
| | EAILTEKISKSRKLENLIKQFPNEKKNGLFGNLIALSLGLQPNFKSNFELSE | |
| | DAKLQFSKDTYEEDLENLLGQIGDEYADLFVAAKNLYDAILLSGILTVNDNS | |
| | TKAPLSASMIKRYDEHHQDLTLLKAFIRENLPEKYKEIFFDKSKNGYAGYID | |
| | GGAKQEEFYKYLKKLLSKIDGSEYFLEKIEREDFLRKQRTFDNGSIPHQIHL | |
| | QELHAILRRQEEFYPFLKENQEKIEKILTFRIPYYVGPLARGNSRFAWASRK | |
| | SDETITPWNFDEVIDKEASAQAFIERMTNNDKNLPEEKVLPKHSLLYEKFTV | |
| | YNELTKVKYVTEGMGEAQFFDANLKQEIFDGLFKKERKVTKKKLLEFLFKEF | |
| | DEFRIVDISGVEKRFNASLGTYHDLLKIIKDKDFLDNEENEKILEDIVLTLT | |
| | LFEDREMIKKRLSKYADLFDKKQLKKLERRRYTGWGRLSAKLINGIRDKQSG | |
| | KTILDYLIDDGFANRNFMQLIHDDNLTFKEEIAKAQVIGESDSLHEVIADLA | |
| | GSPAIKKGILQSLKIVDELVKVMGRYNPENIVVEMARENQTTQKGQRNSRER | |
| | LKRLEESIKNLGSNILKEHPVENTQLQNDKLYLYYLQNGKDMYTGEELDIDR | |
| | LSDYDVDHIIPQSFIKDDSIDNRVLTSSEKARGKSDDVPSEEVVKKMKSFWS | |
| | KLLNAKLISQRKFDNLTKAERGGLTELDKAGFIKRQLVETRQITKHVAQILD | |
| | ERFNTEFDENNKLIRKVKIITLKSKLVSNFRKEFELYKVREINDYHHAHDAY | |
| | LNAVVGKALIKKYPKLESEFVYGDYPVYNSYKMVAKSDKEIGKATAKMFFYS | |
| | NIMNFFKTDIKLADGRIVERPQIETNEETGEIVWDKEKHIATVKKVLSMPQV | |
| | NIVKKTEVQTGGFSKESILPKRNSDKLIARKKDWDTKKYGGFDSPTVAYSVL | |
| | VVADIEKGKAKKLKTVKELVGITIMERSRFEKNPVAFLEDKGYQNIQKEKII | |
| | KLPKYSLFELENGRKRLLASAGELQKGNELALPQKYVTLLYHAKHYEKLKEK | |
| | PEDIEKHKEYVEKHRSEFDEILDQISEFSKRYILADKNLEKIEELYEKNEDA | |
| | SIEELASSFINLLTFTALGAPAAFKFFGKTIDRKRYTSTTEILNATLIHQSI | |
| | TGLYETRIDLSKLGGD | |
| CasEnd-298 | MDKPYSIGLDIGTNSVGWAVVTDEYKVPSKKFKVLGNTDRHSIKKNLLGALL | 258 |
| | FDSGETAEATRLKRTARRRYTRRKNRIRYLQEIFAEEMSKVDDSFFHRLEES | |
| | FLVEEDKRNERHPIFGNIVDEVAYHEKYPTIYHLRKHLADSTEKADLRLIYL | |
| | ALAHIIKFRGHFLIEGDLNAENTDVQKLFEQFVQTYDQTFEESHLSEETVDA | |
| | KAILTDKLSKSRRLENLIAQFPTEKKNGLFGNLLALSLGLQPNFKSNFELAE | |
| | DAKLQFSKDTYDEDLENLLAQIGDQYADLFVAAKNLYDAILLSGILTVNTEI | |
| | TKAPLSASMIKRYDEHHQDLTLLKKFIRQNLPEKYKEIFFDDTKNGYAGYID | |
| | GGASQEEFYKYIKPILEKIDGSEYFLEKIEREDFLRKQRTFDNGSIPHQIHL | |
| | QELKAILRRQEEFYPFLKENQEKIEKILTFRIPYYVGPLARGNSRFAWASRK | |
| | SDETITPWNFEEVVDKEASAQAFIERMTNFDKNLPDEKVLPKHSLLYEKFTV | |
| | YNELTKVKYVTEGMRKPAFFDANQKQEIVDLLFKTNRKVTKKKLKEFLFKEF | |
| | ECFRIVEISGVEDRFNASLGTYHDLLKIIKDKDFLDNEENEDILEDIVLTLT | |
| | LFEDREMIKQRLSKYADLFDKKVLKQLSRRRYTGWGRLSAKLINGIRDKQTG | |
| | KTILDYLIDDGFANRNFMQLIHDDSLTFKEEITKAQVIGEGDSLHEVIADLA | |
| | GSPAIKKGILQSLKIVDELVKVMGRYAPENIVIEMARENQTTQKGQKNSRER | |
| | LKRLEEAIKELGSKILKEHPVENTQLQNDKLYLYYLQNGKDMYTGEELDIDR | |
| | LSDYDVDHIIPQSFIKDDSIDNRVLTSSKKARGKSDDVPSEEVVKKMKSFWR | |
| | QLLNAKLISQRKFDNLTKAERGGLTELDKAGFIKRQLVETRQITKHVAQILD | |
| | SRFNTKYDENDKLIREVKIITLKSKLVSDFRKDFGFYKVREINDYHHAHDAY | |
| | LNAVVGKALIKKYPKLESEFVYGDYPVYNSYKMIAKSEQEIGKATAKYFFYS | |
| | NIMNFFKTDITLANGEIRKRPLIETNEETGEIVWDKTKDFATVKKVLSMPQV | |
| | NIVKKTEVQTGGFSKESILPKRDSDKLIARKKDWDTKKYGGFDSPTVAYSVL | |
| | VIADVEKGKAKKLKTVKELVGITIMERSSFEKNPIAFLEDKGYQNIQKENII | |
| | KLPKYSLFELENGRKRLLASAGELQKGNELALPNKYVTLLYHAKHYEKLKES | |
| | PEDNEEKLEYVEQHRDEFDEILEQISEFSKRYILADKNLEKIKELYKKNEDA | |
| | SIEELAESFINLLTFTALGAPAAFKFFGKTIDRKRYTSTTEVLDATLIHQSI | |
| | TGLYETRIDLSQLGGD | |
| CasEnd-299 | MDKKYSIGLDIGTNSVGWAVITDEYKVPSKKFKVLGNTDRHSIKKNLIGALL | 259 |
| | FDSGETAEATRLKRTARRRYTRRKNRICYLQEIFSNEMAKVDDSFFHRLEES | |
| | FLVEEDKRGERHPIFGNIVDEVAYHEKYPTIYHLRKHLADSTEKADLRLIYL | |
| | ALAHIIKFRGHFLIEGDLNSENSDVQKLFEQLVEVYDQTFEESHLSEETVDA | |
| | KAILTEKLSKSRRLENLIAQFPNEKKNGLFGNLIALSLGLQPNFKSNFELSE | |
| | DAKLQFSKDTYDEDLENLLGQIGDQYADLFLAAKNLYDAILLSGILTVNDEI | |
| | TKAPLSASMIKRYDEHHQDLTLLKAFIRQQLPEKYKEIFFDESKNGYAGYID | |
| | GGASQEEFYKYIKKILEKLDGSEYFLDKIEREDFLRKQRTFDNGSIPHQIHL | |
| | QELHAILRRQEEFYPFLKENQEKIEKILTFRIPYYVGPLARGNSRFAWMSRK | |
| | SDETITPWNFEEVVDKEASAQAFIERMTNFDKNLPEEKVLPKHSLLYEKFTV | |
| | YNELTKVKYVTEGMGKPEFFDAEQKQEIVDGVFKKNRKVTKKQLLDFLFKEF | |
| | DEFRIVDISGVEDAFNASLGTYHDLLKIIKDKDELDNEENEDILEDIILTLT | |
| | LFEDREMIEERLQKYADLFDKKVLKKLERRRYTGWGRLSRKLINGIRDKQSG | |
| | KTILDYLISDGFANRNFMQLIHDDSLSFKEEIAKAQVIGETDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKSKKLKTVKELVGITIMERSAFEKNPVAFLEDKGYQNIQEDLII | |
| | KLPKYSLFELENGRKRLLASAGELQKGNELALPQKYVTLLYHAKHYEKLKEK | |
| | EEDEEKHLEYVEKHRDEFKEIVDQISEFSERYILADKNLEKIKELYSENEEA | |
| | SIEELASSFINLLTFTALGAPAAFKFLGATIDRKRYTSTKEILNATLIHQSI | |
| | TGLYETRIDLSKLGGD | |
| CasEnd-300 | MDKKYSIGLDIGTNSVGWAVITDEYKVPSKKFKVLGNTDRHSIKKNLIGALL | 260 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVQKTFKELLDTYNQLFEESPLDEEEVDA | |
| | KAILTEKISKSRRLENLIAEFPGEKKNGKFGNLLALSLGLTPNFKSNEDLSE | |
| | DAKLQFSKDTYDEDLEELLGQIGDQYADLFVAAKKLYDAILLSGILTVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDELDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRLEEAIKELGSKILKEYPVDNTKLQNEKLYLYYLQNGKDMYTGEPLDIDN | |
| | LSDYDVDHIVPQSFLKDDSIDNKVLVSSEEARGKSDDVPSEAVVRKMKGFWS | |
| | KLLEAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-301 | MKKKYSIGLDIGTNSVGWAVITDEYKVPSKKFKVLGNTDRQSIKKNLLGALL | 261 |
| | FDSGETAEATRLKRTARRRYTRRKNRIRYLQEIFAEEMSKVDDSFFQRLEES | |
| | FLVEEDKRYERHPIFGNIVDEVAYHEKYPTIYHLRKKLADSTDKADLRLIYL | |
| | ALAHIIKFRGHFLIEGDLNSENSDVQKLFIQLVQTYNQLFEENHISEEGVDA | |
| | KAILTDKLSKSRRLENLIALLPNEKKNGLFGNLIALSLGLTPNFKSNFDLAE | |
| | DAKLQFSKDTYDEDLDNLLAQIGDQYADLFLAAKNLSDAILLSDILTVNDEI | |
| | TKAPLSASMIKRYDEHHQDLTLLKKLVRQQLPEKYKEIFFDDSKNGYAGYID | |
| | GGASQEEFYKYIKPILEKMDGTEELLAKINREDLLRKQRTFDNGSIPHQIHL | |
| | KELHAILRRQEDFYPFLKENQEKIEKILTFRIPYYVGPLARGNSRFAWMKRK | |
| | SEETITPWNFEEVVDKEASAQAFIERMTNFDKNLPNEKVLPKHSLLYEYFTV | |
| | YNELTKVKYVTEGMRKPEFFSGEQKKEIVDLLFKTNRKVTVKQLKEDYFKEI | |
| | ECFDSVEISGVEDRFNASLGTYHDLLKIIKDKDFLDNEENEDILEDIVLTLT | |
| | LFEDREMIEERLEKYADLFDKKVLKQLKRRRYTGWGRLSRKLINGIRDKQSG | |
| | KTILDFLKDDGFANRNFMQLIHDDSLTFKEEIQKAQVIGQKDSLHETIANLA | |
| | GSPAIKKGILQSVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKIDENDKLIRDVKIITLKSKLVSDFRKDFQLYKVREINDYHHAHDAY | |
| | LNAVVGTALIKKYPKLESEFVYGDYKVYDVRKMIAKSEQEIGKATAKYFFYS | |
| | NIMNFFKSEITLANGEIRKRPDIETNEETGEIVWDKVKDFATVKKVLSMPQV | |
| | NIVKKTEVQTGGFSKESILPKRNSDKLIARKKDWDTKKYGGFDSPTVAYSVL | |
| | VVADVEKGKSKKLKTVKELVGITIMERSSFEKDPIAFLEAKGYQNIQEDLII | |
| | KLPKYSLFELENGRKRLLASAGELQKGNELALPQHYVTFLYHASNYEKLKGS | |
| | SEDNPQHLEYVEQHRHYFDEILDQISEFSERYILADKNLEKILELYAENEDK | |
| | SINELAENFIHLFTFTSLGAPAAFKFFGTTIDRKRYTSTTEVLDATLIHQSI | |
| | TGLYETRIDLSQLGGD | |
| CasEnd-302 | MDKKYSIGLDIGTNSVGWAVITDEYKVPSKKFKVLGNTDRHSIKKNLIGALL | 262 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVQKLFLQLIQAYDQTFEESPLDEEEIDA | |
| | EAILTEKLSKSRRLENLLAKFPGEKKNGLFGNILKLSVGLTPNFKSNEDLEE | |
| | DAKLQFSKDTYDEDLENLLGQIGDEYADLFVAAKKLYDAILLSGILTVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDELDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRLEENIKELGSNILKEHPVDNTQLQNDKLYLYYLQNGKDMYTGEELDIDN | |
| | LSDYDVDHIVPQSFLKDDSIDNRVLVSSKEARGKSDDVPSEAVVSKMKPFWS | |
| | KLLEAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLINLGAPAAFKYFDTTIDRKRYTSTKEVLDATLIHQSI | |
| | TGLYETRIDLSQLGGD | |
| CasEnd-303 | MKKKYSIGLDIGTNSVGWAVITDEYKVPSKKFKVLGNTDRHSIKKNLLGALL | 263 |
| | FDSGETAEATRLKRTARRRYTRRKNRIRYLQEIFAEEMSKVDDSFFHRLEES | |
| | FLVEEDKRFERHPIFGNIVDEVAYHEKYPTIYHLRKKLADSDEKADLRLIYL | |
| | ALAHIIKFRGHFLIEGDLNAENSDVQKLFIQLVQTYNQLFEESPIDEEGVDA | |
| | KAILTAKLSKSRRLENLIAQLPGEKKNGLFGNLIALSLGLTPNFKSNFDLAE | |
| | DAKLQLSKDTYDEDLDNLLAQIGDQYADLFLAAKNLSDAILLSDILTVNSES | |
| | TKAPLSASMIKRYDEHHQDLTLLKELVREQLPEKYKEIFFDESKNGYAGYID | |
| | GGASQEEFYKYIKPILEKLDGSEELLDKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKEASAQAFIERMTNFDKNLPEEKVLPKHSLLYEYFTV | |
| | YNELTKVKYVTEGMRKPEFLSGEQKEAIVDLLFKKNRKVTVKQLKEDYFKEI | |
| | ECFDSVEISGVEDRFNASLGTYHDLLKIIKDKDFLDNEENEDILEDIVLTLT | |
| | LFEDREMIEERLKKYEHLFDKKVLKQLKRRRYTGWGRLSAKLINGIRDKQSG | |
| | KTILDYLKDDGFANRNFMQLIHDDSLTFKELIQKAQVIGKGDSLHEVIANLA | |
| | GSPAIKKGILQSLKIVDELVKVMGRHNPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIREVKIITLKSKLVSDFRKDFQFYKVREINDYHHAHDAY | |
| | LNAVVGTALIKKYPKLESEFVYGDYKVYDVRKMIAKSDSEIGKATAKYFFYS | |
| | NIMNFFKTEITLANGTIRKRPLIEVNEETGEIVWNKEKDFATVRKVLSMPQV | |
| | NIVKKTEVQTGGFSKESILPKRNSDKLIARKKDWDPKKYGGFDSPTVAYSVL | |
| | VVAKVEKGKAKKLKTVKELVGITIMERSKFEKDPIAFLESKGYKNIQKDLII | |
| | KLPKYSLFELENGRKRLLASAGELQKGNELALPNHYVTLLYLAKNYEKLKGK | |
| | PEDNEQKLEYVEQHKHEFKEIFDQISEFSERYILADKNLEKLKSLYNENEDS | |
| | DISELAENFIHLFTFTSLGAPAAFKFFDKDIDRKRYTSTTEVLNATLIHQSI | |
| | TGLYETRIDLSQLGGD | |
| CasEnd-304 | MKKKYSIGLDIGTNSVGWAVITDEYKVPSKKFKVLGNTDRQSIKKNLLGALL | 264 |
| | FDSGETAEATRLKRTARRRYTRRKNRICYLQEIFAEEMNKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSENTDVQKLFIQLVQTYNQLFEENPINEEGIDA | |
| | KAILSAKLSKSRRLENLIAQIPGEKKNGLFGNLIALSLGLTPNFKSNEDLSE | |
| | DAKLQLSKDTYDEDLDNLLAQIGDQYADLFLAAKNLSDAILLSDILTVNSEI | |
| | TKAPLSASMIKRYDEHHQDLTLLKALVREQLPEKYKEIFFDKSKNGYAGYID | |
| | GGASQEEFYKYIKPILEKMDGTEELLAKINREDFLRKQRTFDNGSIPHQIHL | |
| | GELHAILRRQEKFYPFLKENREKIEKILTFRIPYYVGPLARGNSRFAWMTRK | |
| | SDETITPWNFEEVVDKGASAQAFIERMTNFDKNLPNEKVLPKHSLLYEYFTV | |
| | YNELTKVKYVTEGMGKPEFLSAEQKEEIVDLLFKTNRKVTVKQLKEFYFKEI | |
| | ECFDSVEISGVEDRFNASLGTYHDLLKIIKDKDFLDNEENEDILEDIILTLT | |
| | LFEDREMIEERLKKYADLFDKKVLKKLKRRRYTGWGRLSKKLINGIRDKQSG | |
| | KTILDFLKDDGFANRNFMQLIHDDSLTFKEEIKKAQVIGQTDSLHETIANLA | |
| | GSPAIKKGILQTVKIVDELVKVMGRHKPENIVIEMARENQTTQKGQKNSRER | |
| | MKRLEEGVKELGSQILKEHPVENTQLQNEKLYLYYLQNGKDMYTGQELDIDR | |
| | LSDYDVDHIIPQSFIKDDSIDNKVLTSSEEARGKSDNVPSEEVVKKMKSYWQ | |
| | QLLKAKLISQRKFDNLTKAERGGLSELDKAGFIKRQLVETRQITKHVAQILD | |
| | SRFNTKFDENDKLIRDVKIITLKSKLVSDFRKDFGFYKVREINDYHHAHDAY | |
| | LNAVVGTALIKKYPKLESEFVYGDYKVYDLRKMIGKSEKEIGKATAKMFFYS | |
| | NIMNFFKSEIKLANGEIRKRPVIEVNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKSKKLKTVKELVGITIMERSSFEKNPVKFLEAKGYKNIQKDLII | |
| | KLPKYSLFELENGRKRLLASAGELQKGNELALPSKYVTFLYHASHYEKLKGK | |
| | PEDNEKKREYVEQHLHYFDEIFDQISEFSKRYILADKNLEKIKSTYNKNRNY | |
| | SIREQAESIINLFTFTALGAPAAFKFFDTTIDRKRYTSTKEVLDSTLIHQSI | |
| | TGLYETRIDLSQLGGD | |
| CasEnd-305 | MDKKYSIGLDIGTNSVGWAVITDDYKVPSKKMKVLGNTNKQSIKKNLLGALL | 265 |
| | FDSGETAEATRLKRTARRRYTRRKNRIRYLQEIFSSEMEKVDDSFFHRLKES | |
| | FLVTEDKKNERHPIFGNIVDEVAYHENYPTIYHLRKKLADSTEKADLRLIYL | |
| | AVAHMIKFRGHFLIQGDLNSDNSDVDKLFEQLVETYNELFGESPINTSGVDA | |
| | KTILSARLSKSRRLENLIAQYPGEKKNGLFGNLIALSLGLTPNFKSNEDLAE | |
| | DAKLQFSKDTYDDDLDGLLGQIGDQYADLFLAAKNLSDAILLSDILRVDSVV | |
| | TKAPLSASMIKRYNEHHQDLALLKKLVREQFPEKYKEIFSDQSKNGYAGYID | |
| | GGASQEEFYKFIKPILEKMDETEYFLAKLNREDLLRKQRTFDNGSIPHQIHL | |
| | GELHAILRRQSEHYPFLKENREKIEKILTFRIPYYVGPLARGNSRFAWMKRK | |
| | SNETITPWNFSQVVDKGESAESFITRMTNFDKYLPTEKVLPKHSLLYEYYTV | |
| | YNELTKVKYVTEQRKPKFFSGNVKQRIFDLLFKANRKVTVKQLLEDYKQEFY | |
| | SCDSVEISGLENRFNASLGTYHDLLKIIKDKDFLDNEENQDILEDIVLTLTL | |
| | FEDKEMIRERLKKYAHLEDDKVMKQLERRHYTGWGRLSKKLINGIRDKQSGK | |
| | TILDYLKSDGLSNRNFMQLIHDKSLTFKERIAKANESAQTDSLEEQIAALAG | |
| | SPAIKKGILQTVKVVDELVKVMGHKPENIVIEMARENQTTQEGQKNSRERMK | |
| | RILTGLKELGSDILKKHPVENTQLQNDKLYLYYLQNGRDMYTGQPLDINRLS | |
| | DYDVDHIVPQSFIKDNSFDNKVLTRSDEARGKSDNVPSSEVVKKMKSFWRQL | |
| | LEAKLITQRKYDNLTKAERGGLSELDKAGFIKRQLVETRQITKHVAQILDSR | |
| | MNTKRDKNDKPIRDVKIITLKSKLVSDFRKDFQFYKVREINNYHHAHDAYLN | |
| | AVVGTALIKKYPKLESEFVYGDYKVYDVRKMIAKSEQEIGKATQKRFFYSNI | |
| | MNFFKTDITLANGTIRKRPLIETNTETGEIVWDKGKDLATVRKVLSMPQVNI | |
| | VKKTEVQTGGLYKESILPKREFAKLISRKKRFDSSKYGGEDSPTVAYSVLVI | |
| | AKVEKGKTKKLKTVKTLVGITIMERLSFEKDPVSFLNDKGYKEVKKDKIIKL | |
| | PKYSLFEFENGRRRLLASNGELQKANELVLPAKFVNFLYHAQRISTSKESEN | |
| | DNEKEQEYVDEHRYELQSLFSYIERFAERVILAEKNLEKLKSLFENFESKPI | |
| | RSQCESFIHLFTFTNLGAPAAFKYLNTTIERKRYTSTKSILDSTLIHQSITG | |
| | LYETRIDLSQLGGD | |
| CasEnd-306 | MDKKYSIGLDIGTNSVGWAVITDEYKVPSKKFKVLGNTDRHSIKKNLIGALL | 266 |
| | FDSGETAEATRLKRTARRRYTRRKNRICYLQEIFSNEMAKVDENFFHRLEES | |
| | FLVEEDKRGERHPIFGNIVEEVAYHEKYPTIYHLRKHLADSPEKADLRLVYL | |
| | ALAHIIKFRGHFLIEGDLDAENTDVQKLFKELLEVYDRTFEESHLEEETVDI | |
| | EAILTEKLSKSRRLENLIANFPNEKKNGLFGELLKLIVGLTPNFKSNEDLEE | |
| | DAKLQFSKDTYDEDLEELLGQIGDEYADLFESAKKLYDAILLSGILTVDDNS | |
| | TKAPLSASMVKRYDEHHQDLTLLKQFIRKQLPDKYKEIFFDQSKNGYAGYID | |
| | GGASQEEFYKYLKKLLEKIEGSEYFLEKIEREDFLRKQRTFDNGSIPHQIHL | |
| | QELKAIIRRQGEYYPFLKENQEKIEKILTFRIPYYVGPLARGNSRFAWLSRK | |
| | SDETITPWNFDEIVDKEKSAEAFITRMTNFDKNLPDEKVLPKHSLLYEKFTV | |
| | YNELTKVKYVTEQGKKPEFFDANMKQEIFEGVFKKYRKVTKKQLLDYLKKEF | |
| | DEFRIVDISGVEDRFNASLGTYHDLKKILFDKEFLDDPANEKILEDIILTLT | |
| | LFEDREMIKKRLEKYSDLLTKEQLKKLERRRYTGWGRLSAKLINGIRDKETG | |
| | KTILDYLIDDGYANRNFMQLIHDDNLSFKEEIAKAQVIGETDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VIADIEKGKAKKLKTVKELVGITIMERSKFEKDPVAFLEDKGYQNIQEDNLI | |
| | KLPKYSLFELENGRKRLLASAGELQKGNELALPNHYVTLLYHAKNYEKVKGK | |
| | EEDIEEHLIYVEEHRDEFKELLDQVKEFSERYILADANIEKLKKLYEKNDSA | |
| | SIEELAENFIHLLTFTALGAPAAFKFFGKSIDRKRYTSTKEILNATLIHQSI | |
| | TGLYETRIDLSKLGGD | |
| CasEnd-307 | MKKPYSIGLDIGTNSVGWAVVTDDYKVPSKKMKVLGNTDRSSIKKNLLGALL | 267 |
| | FDSGETAEATRLKRTARRRYTRRKNRILYLQEIFAEEMNKVDDSFFHRLEDS | |
| | FLVEEDKRGERHPIFGNIVEEVAYHEKFPTIYHLRKHLADSTEKADLRLVYL | |
| | ALAHIIKFRGHFLIEGDLNIENTDVQKLFKKFVEVYDRTFEESHLSEETVDA | |
| | ESILTEKVSKSRRLENLIKLFPNEKKNGLFGNLIALSLGLQPNFKTNFKLSE | |
| | DAKLQFSKDTYEEDLENLLGQIGDEYADLFIAAKNLYDAILLSGILTVNDSS | |
| | TKAPLSASMIKRYEEHHEDLTKLKAFIRKQLPEKYKEIFFDETKNGYAGYID | |
| | GGTKQEEFYKYLKKLLSKIDGSEYFLDKIEREDFLRKQRTFDNGSIPHQIHL | |
| | QELHAILRRQAEYYPFLKENQEKIEKILTFRIPYYVGPLARGNSRFAWASRK | |
| | SDETITPWNFDEVIDKEASAEAFIERMTNNDLYLPEEKVLPKHSLLYEKFTV | |
| | YNELTKVKYVTEQMGKAKFFDANMKQEIFDGLFKKYRKVTKKKLLDFLDKEF | |
| | DEFRIVDISGVEKAFNASLGTYHDLLKIIKDKDFLDNEENEDILEDIVLTLT | |
| | LFEDREMIRKRLSKYEDLFTKKQLKKLERRHYTGWGRLSAKLINGIRDKQTG | |
| | KTILDYLIDDGHANRNFMQLIHDDNLSFKEEIAKAQVIGETDSLHEVVAELA | |
| | GSPAIKKGILQSLKIVDELVKVMGRYNPENIVVEMARENQTTNKGQRNSRER | |
| | LKRLEEAIKELGSKILKEHPVENQQLQNDRLYLYYLQNGKDMYTGEELDIDK | |
| | LSQYDVDHIIPQSFIKDDSIDNRVLTSSAKARGKSDDVPSIEVVRKMKSFWS | |
| | KLLNAKLISQRKFDNLTKAERGGLTEDDKAGFIKRQLVETRQITKHVAQILD | |
| | ERFNTEFDENNKLIRDVKIITLKSKLVSQFRKEFELYKVREINDYHHAHDAY | |
| | LNAVVGTALIKKYPKLEPEFVYGDYPKYNSYKLIAKSDKERGKATAKMFFYS | |
| | NIMNFFKTKVKLADGQVIERPVIEVNEETGEIVWDKTKHIATVKKVLSMPQV | |
| | NIVKKTEVQTGGFSKESILPKRDSDKLIARKKKWDTKKYGGFDSPTVAYSVL | |
| | VVADIEKGKAKKLKTVKELVGITIMERSAFEKNPVAFLEDKGYQNIQEEKII | |
| | KLPKYSLFELENGRKRLLASAGELQKGNELALPNKLVTLLYHAKRIEKLDEK | |
| | PEDIEKHLEYVEAHKDEFKELLNQISEFSERYILADKNLEKIEELYEKNDEA | |
| | SIEELASSFINLLTFTALGAPADFKFFGKNIDRKRYTSTKEILNATLIHQSI | |
| | TGLYETRIDLSKLGGD | |
| CasEnd-308 | MKKPYSIGLDIGTNSVGWAVITDDYKVPAKKMKVLGNTDKSHIKKNLLGALL | 268 |
| | FDSGNTAEDRRLKRTARRRYTRRRNRILYLQEIFSEEMAKVDESFFHRLEDS | |
| | FLVPEDKRGERHPIFGNIAEEVAYHKQFPTIYHLRKHLADSSEKADLRLVYL | |
| | ALAHIIKFRGHFLIEGKLDSENTDVQHLFKAFVEVYDNTFEESHLSEQTVDA | |
| | EEILTEKISKSRRLERLLKLFPNEKKNGLFGNFLALIVGLQPNFKSNFELSE | |
| | DAKLQFSKDTYEEDLEGLLGQIGDEYADLFVAAKNLYDAILLSGILTVKDVS | |
| | TKAPLSASMVKRYEEHQADLALLKKFIKQNLPDKYKEVFSDVSKNGYAGYID | |
| | GKTSQEDFYKYLKNLLSKVEGSDYFLDKIEREDFLRKQRTFDNGSIPHQIHL | |
| | QEMHAILRRQGEYYPFLKENQEKIEKILTFRIPYYVGPLARGNSRFAWASRK | |
| | SDEKITPWNFDEIVDKESSAEAFIERMTNYDLYLPNEKVLPKHSLLYEKFTV | |
| | YNELTKVKYITEQFGKYEFFDANMKQEIFDGVFKEERKVTKDKLKEFLDKEF | |
| | DEFRIVDLTGLDKAFNASLGTYHDLLKIIKDKDFLDNSENEKILEDIVLTLT | |
| | LFEDREMIRKRLQKYSDLFTKEQLKKLERRHYTGWGRLSAKLINGIRDKQSN | |
| | KTILDYLIDDGKSNRNFMQLINDDSLSFKEEIAKAQVIGETDNLHQVVSDLA | |
| | GSPAIKKGILQSLKIVDELVKVMGRYNPENIVVEMARENQTTNKGRRNSQQR | |
| | LKRLTDSIKELGSKILKEHPVDNSQLQNDRLFLYYLQNGRDMYTGEELDIDR | |
| | LSQYDIDHIIPQAFIKDDSIDNRVLTSSAKARGKSDDVPSEEVVKKMKSFWS | |
| | KLLSAKLISQRKFDNLTKAERGGLTDDDKAGFIKRQLVETRQITKHVARILD | |
| | ERFNTEFDENNKKIRKVKIVTLKSNLVSQFRKEFELYKVREINDYHHAHDAY | |
| | LNAVVGKALLKKYPKLEPEFVYGEYPKYNSYKLNGKSANERNKATAKMFFYS | |
| | NIMNFFKSDIKLADGEIVERPQIEANDETGEIAWDKTKHFATVRKVLSYPQV | |
| | NIVKKVEEQTGGFSKESILPKGDSDKLIPRKTKWDTKKYGGFDSPIVAYSVL | |
| | VIADIEKGKSKKLKTVKELVGITIMEKHPFEKNPVAFLERKGYRNIQEENII | |
| | KLPKYSLFELENGRRRLLASARELQKGNEAVLPNHLVTLLYHAKNIHKIDEK | |
| | EEPFPKHLEYVEKHRDEFLELLDIIESFSKKYVLAEKNLEKIEELYEKNNEK | |
| | DIEELASSFINLLTFTALGAPAAFKFFDKNIDRKRYTSTAECLNATLIHQSI | |
| | TGLYETRIDLSKLGGD | |
| CasEnd-309 | MDKKYSIGLDIGTNSVGWAVITDEYKVPSKKFKVLGNTDRHSIKKNLLGALL | 269 |
| | FDSGETAEATRLKRTARRRYTRRKNRIRYLQEIFAEEMSKVDDSFFQRLEES | |
| | FLVEEDKRFERHPIFGNIVDEVAYHEEYPTIYHLRKHLADSDEKADLRLIYL | |
| | ALAHIIKFRGHFLIEGPLNSENSDVQKLFIQFVETYNQLFEESPLEEEGVDI | |
| | KAILTAKLSKSRRLENLIANLPNEKKNGLFGNLLALSLGLTPNFKSNFELSE | |
| | DAKLQFSKDTYDEDLENLLAQIGDQYADLFIAAKNLSDAILLSGILTVKTEI | |
| | TKAPLSASMIKRYDEHHQDLTLLKALVREQLPEKYKEIFFDQTKNGYAGYID | |
| | GGASQEDFYKYIKNILEKLDGSEYFLDKINREDFLRKQRTFDNGSIPHQIHL | |
| | QELRAILRRQEEFYPFLKENQEKIEKILTFRIPYYVGPLARGNSRFAWMKRK | |
| | SEETITPWNFEEVVDKEASAQLFIERMTNFDKNLPNEKVLPKHSLLYEKFTV | |
| | YNELTKVKYVTEGMRKPAFFSAGQKEEIVDLLFKKNRKVTVKQLKEYLFKKI | |
| | ECFDSVEISGVEDKFNASLGTYHDLLKIIKDKDFLDNEENEDILEDIVLTLT | |
| | LFEDREMIEKRLAKYAHLFDKKVLKKLKRRRYTGWGRLSAKLINGIRDKQTG | |
| | KTILDYLIDDGFANRNFMQLIHDDSLTFKEEIKKAQVIGQGDSLHEVIANLA | |
| | GSPAIKKGILQSLKIVDELVKVMGRHSPENIVIEMARENQTTQKGQKNSRER | |
| | LKRLEEVIKKLGSKILKEHPVDNTQLQNDKLYLYYLQNGRDMYTGQELDIDR | |
| | LSDYDVDHIIPQSFIKDDSIDNRVLTSSGKARGKSDDVPSEEVVKKMKNFWR | |
| | QLLNSKLISQRKFDNLTKAERGGLTELDKAGFIKRQLVETRQITKHVAQILD | |
| | SRFNTKYDENDKLIREVKIITLKSKLVSDFRKDFGFYKVREINDYHHAHDAY | |
| | LNAVVGTALIKKYPKLESEFVYGDYKVYNVRKMIAKSEQEIGKATAKYFFYS | |
| | NIMNFFKSDVTLANGEIRKRPLIETNEETGEIVWDKTKDIATVKKVLSMPQV | |
| | NIVKKTEVQTGGFSKESILPKRNSDKLIARKKDWDTKKYGGFDSPTVAYSVL | |
| | VVADVEKGKAKKLKTVKELVGITIMERSAFEKDPVGFLEDKGYQNIQKDLII | |
| | KLPKYSLFELENGRKRLLASAGELQKGNELALPQKYVILLYHAKNYEKLKGS | |
| | PEDNEKHLEYVEQHRHEFDEILNQIIEFSERYILADKNLEKIEELYKENNDS | |
| | PIEELASSFLNLFTFTSLGAPAAFKFFGTDIDRKRYTSTTEILNATLIHQSI | |
| | TGLYETRIDLSQLGGD | |
| CasEnd-310 | MDKPYSIGLDIGTNSVGWAVITDEYKVPSKKFKVLGNTDRHSIKKNLIGALL | 270 |
| | FDSGETAEATRLKRTARRRYTRRKNRIRYLQEIFAEEMNKVDDSFFHRLEES | |
| | FLVEEDKRGERHPIFGNIVDEVAYHEKYPTIYHLRKHLADSPEKADLRLIYL | |
| | ALAHIIKFRGHFLIEGDLKAENTDVQKLFEDLVQTYNNTFEESALSEELVDA | |
| | FAILTAKVSKSRRLENLIKDYPNEKKNGLFGNLIALSLGLTPNFKTNFELSE | |
| | DAKLQFSKDTYDEDLENLLGQIGDQYADLFLAAKNLYDAILLSGILTVKTEI | |
| | TKAPLSASMVKRYDEHHQDLTLLKQFIRQNLPEKYKEIFFDKSKNGYAGYID | |
| | GGASQEEFYKYIKKILEKIDGSEYFLDKIEREDFLRKQRTFDNGSIPHQIHL | |
| | QELKAILRRQEKFYPFLKENQEKIEKILTFRIPYYVGPLARGNSRFAWLSRK | |
| | SDETITPWNFEEIVDKEASAQAFIERMTNYDKNLPEEKVLPKHSLLYEKFTV | |
| | YNELTKVKYVTEGMRKPAFLDAGQKQEIVDLLFKKNRKVTVKQLKEFLFKEI | |
| | DCFDIVEISGVEDRFNASLGTYHDLLKIIKDKDFLDNEENEEILEDIVLTLT | |
| | LFEDREMIEERLSKYADLFDKKVLKKLERRRYTGWGRLSAKLINGIRDKQSG | |
| | KTILDYLIDDGFANRNFMQLIHDDSLSFKEEIQKAQVIGQTDSLHEVIANLA | |
| | GSPAIKKGILQSIKIVDELVKVMGRHEPENIVVEMARENQTTQKGQKNSRER | |
| | LKRLEEAHKKLGSNILKEHPVENTQLQNDKLYLYYLQNGKDMYTGEELDIDR | |
| | LSDYDVDHIIPQSFIKDDSIDNRVLTSSKKARGKSDNVPSEDVVKKMKNFWE | |
| | KLLNAKLISQRKFDNLTKAERGGLTELDKAGFIKRQLVETRQITKHVAQILD | |
| | SRFNTKYDENDKLIRNVKIITLKSKLVSDFRKEFGFYKVREINDYHHAHDAY | |
| | LNAVVGTALIKKYPKLESEFVYGDYKVYNLYKMIAKSEQEIGKATAKYFFYS | |
| | NIMNFFKQEITLANGEIRKRPLIETNEETGEIVWDKAKDFATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSSFEKDPIAFLEDKGYKNIQKENII | |
| | KLPKYSLFELENGRKRLLASAGELQKGNELALPQKYVTLLYHAHHYEKLKGS | |
| | PEDNEKHLEYVEQHRHEFDEILEQIIEFSERYILADKNLEKIQELYTKNSNA | |
| | DINELAESFINLLTFTALGAPAAFKFFGKDIDRKRYTSTTEILNATLIHQSI | |
| | TGLYETRIDLSQLGGD | |
| CasEnd-311 | MKKKYSIGLDIGTNSVGWAVITDEYKVPSKKFKVLGNTDRQSIKKNLIGALL | 271 |
| | FDSGETAEATRLKRTARRRYTRRKNRIRYLQEIFAEEMNKVDDSFFHRLEES | |
| | FLVEEDKRGERHPIFGNIVDEVAYHEKYPTIYHLRKKLVDSTEKADLRLIYL | |
| | ALAHMIKFRGHFLIEGDLNSENSDVQKLFIDLVQTYNQIFEESHLSESGVDA | |
| | KAILTEKLSKSRRLENLIAQFPNEKKNGLFGNLIALSLGLTPNFKSNENLSE | |
| | DAKLQFSKDTYDEDLENLLGQIGDEYADLFLAAKNLSDAILLSGILRVDDES | |
| | TKAPLSASMIKRYDEHHQDLTLLKALVRKQLPEKYKEIFFDKSKNGYAGYID | |
| | GGASQEEFYKYIKPILEKMDGTEYLLEKLEREDLLRKQRTFDNGSIPHQIHL | |
| | QELHAILRRQEEYYPFLKENQEKIEKILTFRIPYYVGPLARGNSRFAWMTRK | |
| | SEETITPWNFEEVVDKGASAQAFIERMTNFDKNLPEEKVLPKHSLLYEYFTV | |
| | YNELTKVKYVTEGMGKAEFLDANVKKEIVDGLFKKNRKVTVKKLKDFYFKEF | |
| | DEFRIVDISGVEDRFNASLGTYHDLLKIIKDKEFLDNEENEDILEDIVLTLT | |
| | LFEDREMIEKRLEKYANLFDKKQMKKLKRRRYTGWGRLSKKLINGIRDKQSG | |
| | KTILDFLIDDGFANRNFMQLIHDDSLTFKDEIKKAQVIGQSDSLHEQIADLA | |
| | GSPAIKKGILQTVKVVDELVKVMGRHKPENIVIEMARENQTTQKGQRNSRER | |
| | LKRIEEGIKELGSQILKEHPVENTQLQNDKLYLYYLQNGRDMYTGEELDIDR | |
| | LSDYDVDHIVPQSFIKDDSIDNKVLTRSKKARGKSDDVPSEEVVKKMKSYWR | |
| | QLLKAKLITQRKFDNLTKAERGGLTELDKAGFIKRQLVETRQITKHVAQILD | |
| | SRMNTERDENDKLIRDVKVITLKSKLVSDFRKDFQFYKVREINDYHHAHDAY | |
| | LNAVVGTALIKKYPKLESEFVYGDYPVYNSRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-312 | MKKPYSIGLDIGTNSVGWAVVTDDYKVPAKKMKVLGNTDKSHIKKNLLGALL | 272 |
| | FDSGNTAEDRRLKRTARRRYTRRRNRILYLQEIFSEEMGKVDDSFFHRLEDS | |
| | FLVTEDKRGERHPIFGNLEEEVAYHENFPTIYHLRKYLADNPEKADLRLVYL | |
| | ALAHIIKFRGHFLIEGKLDTRNNDVQRLFQEFLAVYDNTFENSSLQEQNVQV | |
| | EEILTDKISKSAKKDRVLKLFPNEKSNGRFAEFLKLIVGNQADFKKHFELEE | |
| | KAPLQFSKDTYEEDLEVLLAQIGDEYAELFLSAKKLYDSILLSGILTVTDVS | |
| | TKAPLSASMIKRYNEHQMDLAQLKQFIRQKLPDKYNEVESDVSKDGYAGYID | |
| | GKTNQEAFYKYLKKLLNKIEGSGYFLDKIEREDFLRKQRTFDNGSIPHQIHL | |
| | QEMRAIIRRQAEFYPFLKENQDKIEKILTFRIPYYVGPLARGKSDFAWLSRK | |
| | SADKITPWNFDEIVDKESSAEAFINRMTNYDLYLPNEKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASYGTYHDLRKILKDKDFLDNSKNEKILEDIVLTLT | |
| | LFEDREMIRKRLENYSDLLTKEQLKKLERRHYTGWGRLSAKLIHGIRNKESR | |
| | KTILDYLIDDGNSNRNFMQLINDDALSFKEEIAKAQVIGETDNLNQVVSDIA | |
| | GSPAIKKGILQSLKIVDELVKIMGGHQPENIVVEMARENQFTNQGRRNSQQR | |
| | LKGLTDSIKELGSQILKEHPVENSQLQNDRLFLYYLQNGRDMYTGEELDIDY | |
| | LSQYDIDHIIPQAFIKDDSIDNRVLTSSAEARGKSDDVPSKDVVKKMKSYWS | |
| | KLLSAKLITQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLKKYPKLEPEFVYGDYPKFNSHKIVSESKEEENKATAKKFFYS | |
| | NIMNFFKKDVKLADGQIVERPMIERNDENGEIVWDKDKHISNVKKVLSYPQV | |
| | NIVKKVEEQTGGFSKESILPKGNSDKLIPRKTKWDTKKYGGFDSPIVAYSVL | |
| | VIADIEKGKSKKLKTVKELVGITIMEKMTFEKNPVAFLERKGYQNIQEENII | |
| | KLPKYSLFELENGRKRLLASAKELQKGNEIVLPNHLVTLLYHAKNIHKIDEK | |
| | TEDIPKHLEYVEKHKDEFKELLDVVSNFSKKYTLAEGNLEKILELYAQNNSA | |
| | DIEELASSFINLLTFTALGAPATFKFFDKNIDRKRYTSTTEILNATLIHQSI | |
| | TGLYETRIDLSKLGGD | |
| CasEnd-313 | MKKPYSIGLDIGTNSVGWAVVTDDYKVPAKKMKVLGNTDKSHIKKNLLGALL | 273 |
| | FDSGNTAEDRRLKRTARRRYTRRRNRILYLQEIFSEEMGKVDDSFFHRLEDS | |
| | FLVPEDKRGERHPIFGNLEEEVKYHENFPTIYHLRKYLADNPEKADLRLVYL | |
| | ALAHIIKFRGHFLIEGKFDTRNNDVQRLFQEFLAVYDNTFENSSLQEQNVQV | |
| | EEILTDKISKSAKKDRVLKLFPNEKSNGRFAEFLKLIVGNQADFKKHFELEE | |
| | KAPLQFSKDTYEEDLEVLLGQIGDDYAELFLSAKKLYDSILLSGILTVTDVS | |
| | TKAPLSASMIKRYNEHQMDLTQLKQFIRQKLSDKYNEVFSDVSKDGYAGYID | |
| | GKTSQEAFYKYLKKLLNKIEGSEYFLDKIEREDFLRKQRTFDNGSIPHQIHL | |
| | QEMRAIIRRQAEFYPFLAENQDKIEKILTFRIPYYVGPLARGKSDFAWLSRK | |
| | SAEKITPWNFDEIVDKESSAEAFINRMTNYDLYLPNEKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASLGTYHDLLKILNDKDFLDNSKNEKILEDIVLTLT | |
| | LFEDREMIRKRLENYSDLLTKEQLKKLERRHYTGWGRLSAKLIHGIRNKESR | |
| | KTILDYLIDDGNSNRNFMQLINDDALSFKEEIAKAQVIGETDNLNQVVSDLA | |
| | GSPAIKKGILQSLKIVDELVKIMGTHQPENIVVEMARENQFTNQGRRNSQQR | |
| | LKGLTDSIKELGSQILKEHPVENSQLQNDRLFLYYLQNGRDMYTGEELDIDY | |
| | LSQYDIDHIIPQAFIKDNSIDNRVLVSSKEARGKSDDVPSKDVVRKMKSYWS | |
| | KLLSAKLISQRKFDNLTKAERGGLTDDDKAGFIKRQLVETRQITKHVARILD | |
| | ERFNTETDENNKKIRQVKIVTLKSNLVSNFRKEFELYKVREINDYHHAHDAY | |
| | LNAVVGKALLGKYPQLEPEFVYGEYPKFNSHKFVRKSDKEENKATAKKFFYS | |
| | NIMNFFKKDIKLADGSIVERPVVERNDETGEIIWDKDKHISNVKKVLSYPQV | |
| | NIVKKVEEQTGGFSKESILPKGNSDKLIPRKTKWDTKKYGGFDSPIVAYSIL | |
| | VIADIEKGKSKKLKTVKELVGITIMEKMTFERDPVAFLERKGYRNIQEENII | |
| | KLPKYSLFELENGRKRLLASARELQKGNEIVLPNHLGTLLYHAKNIHKVDEK | |
| | EEEIPKHLEYVDKHRDEFKELLDVVSNFSKKYILAEGNLEKIKELYAQNNSE | |
| | SIEELASSFINLLTFTAIGAPAAFKFFDKNIDRKRYTSTTEILNATLIHQSI | |
| | TGLYETRIDLSKLGGD | |
| CasEnd-314 | MKKKYSIGLDIGTNSVGWAVITDEYKVPSKKFKVLGNTDRKSIKKNLLGALL | 274 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVQKLFIQLVQTYNQLFEENPINEEEVDA | |
| | KAILSAKLSKSRRLENLIAQFPNEKKNGLFGNLIALSLGLTPNFKSNFDLAE | |
| | DAKLQLSKDTYDEDLENLLAQIGDQYADLFLAAKNLSDAILLSDILTVNDES | |
| | TKAPLSASMIKRYDEHHQDLTLLKNLVRQQLPEKYKEIFFDKSKNGYAGYID | |
| | GGASQEEFYKYIKPILEKLDGTEEFLAKINREDFLRKQRTFDNGSIPHQIHL | |
| | NELHAILRRQEDFYPFLKENQEKIEKILTFRIPYYVGPLARGNSRFAWLTRK | |
| | SDETITPWNFEEVVDKGASAQAFIERMTNFDKNLPEEKVLPKHSLLYEYFTV | |
| | YNELTKVKYITEGMRKPEFLSSEQKEAIVDLLFKKNRKVTVKQLKEDYFKKI | |
| | ECFDSVEISGVEDRFNASLGTYHDLLKIIKDKDFLDNEENEDILEDIILTLT | |
| | LFEDREMIEERLKKYAHLFDKKVLKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQSLKIVDELVKVMGRHNPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENNKLIRDVKIITLKSKLVSDFRKDFGLYKVREINDYHHAHDAY | |
| | LNAVVGTALIKKYPKLESEFVYGDYKVYDVRKMIAKSEQEIGKATAKYFFYS | |
| | NIMNFFKSEVTLANGTIRKRPKIETNEETGEIVWDKEKDFATVKKVLSMPQV | |
| | NIVKKTEVQTGGFSKESILPKRNSDKLIARKKDWDTKKYGGFDSPTVAYSVL | |
| | VVAKVEKGKAKKLKTVKELVGITIMERSSFEKDPIAFLEAKGYQDIREELII | |
| | KLPKYSLFELENGRKRMLASAGELQKGNELALPNHYVTLLYHASHYEKLKGK | |
| | SEDIEHKREYVEQHRHEFDEIFEQISEFSERYILADKNLEKIKSLFDENTDK | |
| | DIRELAENFIHLFTFTALGAPAAFKFFDTTIDRKRYTSTTEILDATLIHQSI | |
| | TGLYETRIDLSKLGGD | |
| CasEnd-315 | MKKPYSIGLDIGTNSVGWAVITDEYKVPSKKFKVLGNTDRQSIKKNLLGALL | 275 |
| | FDSGETAEATRLKRTARRRYTRRKNRIRYLQEIFAEEMSKVDDSFFHRLEES | |
| | FLVEEDKDDERHPIFGNIVDEVAYHENYPTIYHLRKHLADSTEKADLRLIYL | |
| | ALAHIIKFRGHFLIEGDLNSENTDVQKLFIKLVQTYNQTFEENPLSEAEIDA | |
| | KAILTAKLSKSRRLENLLAKFPNEKRNGLFGNLLALSLGLTPNFKSNFELSE | |
| | DAKLQISKDTYDEDLENLLAQIGDQYADLFVAAKNLYDAILLSGILTVNDES | |
| | TKAPLSASMIKRYDEHHQDLTLLKKLVRKQLPEKYKEIFFDESKNGYAGYID | |
| | GGASQEEFYKYIKPILSKLDGSEYFLDKIEREDFLRKQRTFDNGSIPHQIHL | |
| | NELHAILRRQEDYYPFLKENREKIEKILTFRIPYYVGPLARGNSRFAWAKRK | |
| | SDETITPWNFEEVVDKEASAQAFIERMTNYDKNLPEEKVLPKHSLLYEKFTV | |
| | YNELTKVKYITEGMKKPEFFSAEQKKEIVDLLFKKNRKVTVKKLKEFLFKKV | |
| | ECFDSVELSGVEDAFNASLGTYHDLLKILKDKDFLDNEANEDILEDIVLTLT | |
| | LFEDREMIEQRLLKYADLFDKKVLKKLKRRRYTGWGRLSAKLINGIRDKQSG | |
| | KTILDYLIDDGFANRNFMQLIHDDSLSFKEEIKKAQVIGQTDSLHEVIANLA | |
| | GSPAIKKGILQSLKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEFDENDKLIREVKIITLKSKLVSDFRKDFGFYKVREINDYHHAHDAY | |
| | LNAVVGTALIKKYPKLESEFVYGDYKVYNVRKLIGKSDKEIGKATAKYFFYS | |
| | NIMNFFKTEITLANGTIRKRPLIETNEETGEIVWDKEKDFATVKKVLSMPQV | |
| | NIVKKTEVQTGGFSKESILPKRDSDKLIARKKDWDTKKYGGFDSPTVAYSVL | |
| | VVADVEKGKAKKLKTVKELVGITIMERSSFEKDPIAFLEDKGYHNIRKDNMI | |
| | KLPKYSLFELENGRKRLLASAGELQKGNELALPQKYVTLLYLASHYEKGKGK | |
| | SEDKSNKLEFVKQHRHEFDEIFDQIEEFSKRYILADKNLEKILEAYKENEEF | |
| | SISELAENFIHLFTFTSLGAPAAFKFFGKDIDRKRYTSTTEILNATLIHQSI | |
| | TGLYETRIDLSKLGGD | |
| CasEnd-316 | MDKPYSIGLDIGTNSVGWAVITDEYKVPSKKFKVLGNTDRHSIKKNLLGALL | 276 |
| | FDSGETAEATRLKRTARRRYTRRKNRILYLQEIFAEEMSKVDDSFFHRLEES | |
| | FLVEEDKRHDRHPIFGNIVEEVAYHENYPTIYHLRKKLADSTEKADLRLVYL | |
| | ALAHIIKFRGHFLIEGDLNSENTDVQKLFEKLVQTYDQTFEESHLSEETVDA | |
| | KEILTDKVSKSRRLENLIKQFPNEKKNGLFGNLIALSLGLQPNFKTNEDLSE | |
| | DAKLQFSKDTYDEDLENLLGQIGDDYADLFAAAKNLYDAILLSGILTVDTEI | |
| | TKAPLSASMIKRYDEHHQDLTLLKKFIRQNLPEKYKEIFFDESKNGYAGYID | |
| | GGAKQEEFYKYIKNILNKIDGSEYFLAKINREDFLRKQRTFDNGSIPHQIHL | |
| | QELKAILRRQGDYYPFLKENREKIEKILTFRIPYYVGPLARGNSRFAWLKRK | |
| | SDETITPWNFEEVVDKEASAQAFIERMTNYDKNLPEEKVLPKHSLLYEKFTV | |
| | YNELTKVKYVTEGMRKPAFFDANQKQEIVDLLFKTNRKVTVKKLKEFLFKEF | |
| | EEFDIVEISGVEKSFNASLGTYHDLLKIIKDKDFLDNPENEEILEDIVLTLT | |
| | LFEDREMIEERLSKYAHLFDKKVLKKLERRRYTGWGRLSAKLINGIRDKQSG | |
| | KTILDYLIDDGFANRNFMQLIHDDSLDFKEEIAKAQVIGETDSLHETIANLA | |
| | GSPAIKKGILQSLKIVDELVKVMGRHAPENIVIEMARENQTTQKGQRNSRER | |
| | LKRLEEAIKNLGSKILKEHPVENTQLQNDKLYLYYLQNGKDMYTGEELDIDR | |
| | LSDYDVDHIIPQSFIKDDSIDNRVLTSSAEARGKSDDVPSEEVVKKMKSFWH | |
| | KLLKSKLISQRKFDNLTKAERGGLSELDKAGFIKRQLVETRQITKHVAQILD | |
| | SRFNTKYDENDKLIRDVKIITLKSKLVSDFRKDFQLYKVREINDYHHAHDAY | |
| | LNAVVGTALIKKYPKLESEFVYGDYPVYNSYKMIAKSEQEIGKATAKYFFYS | |
| | NIMNFFKKDVTLANGEIRKRPLIETNEETGEIVWDKEKDFATVRKVMSMPQV | |
| | NIVKKTEVQTGGFSKESILPKRNSDKLIARKKDWDTKKYGGFDSPTVAYSVL | |
| | VVADIEKGKVKKLKTVKELVGITIMERSSFEKDPVAFLENKGYQNIQKENII | |
| | KLPKYSLFELENGRKRLLASAGELQKGNELALPNHYVTLLYHAKNYEKLKES | |
| | PEDNEKHLEYVEQHRDEFDELLDQISEFSERYILADKNLEKILELYSQNENS | |
| | DIEELASSFINLLTFTALGAPAAFKFFGKEIDRKRYTSTTEILNATLIHQSI | |
| | TGLYETRIDLSQLGGD | |
| CasEnd-317 | MDKKYSIGLDIGTNSVGWAVITDEYKVPSKKFKVLGNTDRHSIKKNLIGALL | 277 |
| | FDSGETAEATRLKRTARRRYTRRKNRIRYLQEIFSNEMAKVDDSFFHRLEES | |
| | FLVEEDKKHERHPIFGNIVDEVAYHEKYPTIYHLRKKLADSDEKADLRLIYL | |
| | ALAHMIKFRGHFLIEGDLNSDNSDVDKLFKQLVQTYNQLFEENPINEEGVDA | |
| | KAILSARLSKSRRLENLIAQFPGEKKNGLFGNLIALSLGLTPNFKSNEDLAE | |
| | DAKLQLSKDTYDEDLENLLAQIGDQYADLFLAAKNLSDAILLSDILTVNSEI | |
| | TKAPLSASMVKRYDEHHQDLTLLKALVRQQLPEKYKEIFFDQSKNGYAGYID | |
| | GGASQEEFYKYIKPILEKLDGTEELLDKIEREDFLRKQRTFDNGSIPHQIHL | |
| | GELHAILRRQEKYYPFLKENREKIEKILTFRIPYYVGPLARGNSRFAWMTRK | |
| | SDETITPWNFEEVVDKGASAQSFIERMTNFDKNLPNEKVLPKHSLLYEKFTV | |
| | YNELTKVKYVTEGMRKPEFLSGEQKQEIVDLLFKKNRKVTVKQLKEYYFKKI | |
| | ECFDSVEISGVEDRFNASLGTYHDLLKIIKDKDFLDNEENEDILEDIVLTLT | |
| | LFEDREMIEERLKKYADLFDKKVLKQLKRRRYTGWGRLSRKLINGIRDKQSG | |
| | KTILDFLKSDGFANRNFMQLIHDDSLTFKEEIEKAQVSGQGDSLHEQIANLA | |
| | GSPAIKKGILQSLKIVDELVKVMGRHKPENIVIEMARENQTTQKGQKNSRER | |
| | MKRLEEAIKELGSNILKEHPVENTQLQNEKLYLYYLQNGKDMYTGEELDIDR | |
| | LSDYDVDHIVPQSFLKDDSIDNKVLTSSAKARGKSDNVPSEEVVKKMKNYWK | |
| | QLLDAKLITQRKFDNLTKAERGGLTELDKAGFIKRQLVETRQITKHVAQILD | |
| | SRFNTKYDENDKLIRDVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKEKDFATVRKVLSMPQV | |
| | NIVKKTEVQTGGFSKESILPKRDSDKLIARKKDWDTKKYGGFDSPTVAYSVL | |
| | VVAKVEKGKAKKLKTVKELVGITIMERSSFEKNPIDFLEAKGYKEVQKDLII | |
| | KLPKYSLFELENGRKRMLASAGELQKGNELALPSKYVEFLYLASHYEKLKGS | |
| | PEDNEQKQLFVEQHKEYFDEIIEQISEFSKRYILADANLEKIKSLYEKNRDK | |
| | PIEEQAESFINLLTFTALGAPAAFKFFDTTIDRKRYTSTKEILNATLIHQSI | |
| | TGLYETRIDLSQLGGD | |
| CasEnd-318 | MKKKYSIGLDIGTNSVGWAVITDEYKVPSKKFKVLGNTDRQSIKKNLIGALL | 278 |
| | FDSGETAEATRLKRTARRRYTRRKNRIRYLQEIFAEEMSKVDDSFFHRLEES | |
| | FLVEEDKKNERHPIFGNIVDEVAYHEKYPTIYHLRKKLADSTEKADLRLIYL | |
| | ALAHIIKFRGHFLIEGDLNSENSDVQKLFKQLVQTYNQLFEENPLNESGVDA | |
| | KAILTAKLSKSRRLENLIAQFPGEKKNGLFGNLIALSLGLTPNFKSNFDLAE | |
| | DAKLQLSKDTYDDDLENLLAQIGDQYADLFLAAKNLSDAILLSDILTVNDES | |
| | TKAPLSASMIKRYDEHHQDLTLLKALVRQQLPEKYKEIFFDDSKNGYAGYID | |
| | GGASQEDFYKFIKPILEKMDGSEDFLAKLNREDFLRKQRTFDNGSIPHQIHL | |
| | DELHAILRRQEEFYPFLKENREKIEKILTFRIPYYVGPLARGNSRFAWMTRK | |
| | SEETITPWNFEEVVDKEASAQAFIERMTNFDKNLPNEKVLPKHSLLYEYFTV | |
| | YNELTKVKYVTEGMRKPEFLSGEQKEAIVDLLFKKNRKVTVKQLKEDYFKKI | |
| | ECFDSVEISGVEDRFNASLGTYHDLLKIIKDKDFLDNEENEDILEDIVLTLT | |
| | LFEDKEMIEKRLSKYAHLFDKKVLKQLKRRRYTGWGRLSRKLINGIKDKQSG | |
| | KTILDFLKSDGFANRNFMQLIHDDSLTFKEEITKAQVKGQGDSLHEQIANLA | |
| | GSPAIKKGILQSVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRENTEYDENNKLIREVKIITLKSKLVSDFRKDFQFYKVREINNYHHAHDAY | |
| | LNAVVGTALIKKYPKLESEFVYGDYKVYNVRKMIIKSEQEIGKATAKYFFYS | |
| | NIMNFFKSDITLANGEIRKRPLIETNEETGEIVWDKTKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKDPVAFLETKGYKNIRKELII | |
| | KLPKYSLFELENGRKRLLASAGELQKGNELALPNKYVTLLYHAHHYEKLKGS | |
| | EEDKEKKLSFVEQHRDYFDEIFDQIIEFSKRYILADKNLEKIKELYSNKEVK | |
| | SISELAENFIHLLTFTSLGAPAAFKFFDTTIDRKRYTSTTEVLNATLIHQSI | |
| | TGLYETRIDLSQLGGD | |
| CasEnd-319 | MDKKYSIGLDIGTNSVGWAVITDEYKVPSKKFKVLGNTDRHSIKKNLIGALL | 279 |
| | FDSGETAEATRLKRTARRRYTRRKNRICYLQEIFSNEMAKVDESFFHRLEES | |
| | FLVEEDKRNERHPIFGNIVDEVAYHEKYPTIYHLRKKLVDSDEKADLRLIYL | |
| | ALAHMIKFRGHFLIEGDLNSDNSDVQKLFVQLVQTYNQLFEESPIEAEGVDA | |
| | KAILSEKLSKSRRLENLIAQLPGEKKNGLFGNLIALSLGLTPNFKSNFDLSE | |
| | DAKLQLSKDTYDDDLEELLGQIGDQYADLFLAAKNLSDAILLSGILRVNTES | |
| | TKAPLSASMIKRYDEHHQDLTLLKELVRKQLPEKYKEIFFDQSKNGYAGYID | |
| | GGASQEEFYKYIKKILEKMDGTEELLAKLEREDLLRKQRTFDNGSIPHQIHL | |
| | GELHAILRRQEEYYPFLKENREKIEKILTFRIPYYVGPLARGNSRFAWMSRK | |
| | SDETITPWNFDEVVDKGASAEKFIERMTNFDKNLPDEKVLPKHSLLYEYFTV | |
| | YNELTKVKYVTEGGRKPEFLDGEQKKEIVDLLFKKNRKVTVKQLKEYYFKEF | |
| | DCFDIVEISGVEDRFNASLGTYHDLLKIIKDKEFLDNEENEEILEDIVLTLT | |
| | LFEDREMIKERLEKYADLFDKKVMKQLKRRRYTGWGRLSKKLINGIRDKQSG | |
| | KTILDFLKEDGFTNRNFMQLIHDDNLTFKEEIDKAQVTGKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-320 | MDKPYSIGLDIGTNSVGWAVVTDEYKVPSKKFKVLGNTDRKSIKKNLLGALL | 280 |
| | FDSGETAEATRLKRTARRRYTRRKNRILYLQEIFAEEMNKVDESFFHRLEES | |
| | FLVEEDKRGERHPIFGNIVEEVAYHEKYPTIYHLRKHLADSDEKADLRLVYL | |
| | ALAHIIKFRGHFLIEGDLDSENTDVQALFKELLEIYDRTFEESPLEEETVDA | |
| | ESILTEKISKSRRLENLLAEFPGEKKNGFFGNFLKLIVGLTPNFKSNFGLEE | |
| | DAKLQFSKDTYDEDLEELLGQIGDEYADLFVAAKNLYDAILLSGILTVKDNS | |
| | TKAPLSASMVKRYDEHHQDLTLLKQFIRKNLPEKYKEIFFDQSKNGYAGYID | |
| | GGASQEDFYKYLKKLLEKIDGSEYFLDKIEREDFLRKQRTFDNGSIPHQIHL | |
| | QELKAIIRRQGEYYPFLKENQEKIEKILTFRIPYYVGPLARGNSRFAWLSRK | |
| | SDETITPWNFDEVVDKEKSAEAFIERMTNFDKNLPDEKVLPKHSLLYEKFTV | |
| | YNELTKVKYVTEGGKKPNFFDANLKQEIFDNVFKKYRKVTKKQLLDFLKKEF | |
| | DEFRIVDISGVEDRFNASLGTYHDLLKILDGKDFLDDPENEEILEDIIKTLT | |
| | LFEDREMIKKRLEKYSDLFDKEQLKKLERRRYTGWGRLSAKLINGIRDKETG | |
| | KTILDYLIDDGNANRNFMQLIHDDSLSFKEEIAKAQVIGDSESLHEVIANLA | |
| | GSPAIKKGILQSLKIVDELVKVMGRYEPENIVVEMARENQTTQKGQKNSRER | |
| | MKRLEESIKELGSKILKEHPTENTKLQNDKLYLYYLQNGKDMYTGEPLDIDN | |
| | LSDYDVDHIVPQSFLKDDSIDNRVLVSSAKARGKSDDVPSEEIVKKMKPFWK | |
| | KLLEAKLITQRKYDNLTKAERGGLTELDKAGFIKRQLVETRQITKHVAQILD | |
| | ERFNTEVDENGKLIRDVKIVTLKSKLVSQFRKEFELYKVREINNYHHAHDAY | |
| | LNAVVGKALIKKYPKLESEFVYGDYPVYDVKKLIRKSSREIGKATAKMFFYS | |
| | NIMNFFKSDVKLADGDVRERPDIEVNEETGEIAWDKEKHIATVKKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSRFEKNPVAFLEDKGYKNIQKENII | |
| | KLPKYSLFELENGRKRLLASAGELQKGNELALPNHYVTLLYHAKHYEKLKGK | |
| | PEDIEESRNYVEEHRDEFDELLDQISEFSKRYILADANLEKIKKLYEKNEDA | |
| | SIEELASSFINLLTFTALGAPAAFKFFGKNIDRKRYTSTKEILNATLIHQSI | |
| | TGLYETRIDLSKLGED | |
| CasEnd-321 | MDKSYSIGLDIGTNSVGWAVITDEYKVPSKKFKVLGNTDRHSIKKNLLGALL | 281 |
| | FDSGETAEATRLKRTARRRYTRRKNRIRYLQEIFAEEMSKVDDSFFHRLEES | |
| | FLVEEDKRGERHPIFGNIVDEVAYHEEYPTIYHLRKYLADSTEKADLRLIYL | |
| | ALAHIIKFRGHFLIEGDLNPENTDVQKLFIQFVETYDNTFEESHLSEETVDA | |
| | KAILTDKLSKSRRLENLIKQFPGEKKNGLFGNLIALSLGLTPNFKINFELSE | |
| | DAKLQFSKDTYDEDLENLLAQIGDDYADLFVAAKNLYDAILLSGILTVNTEI | |
| | TKAPLSASMIKRYDEHHQDLTLLKAFVREQLPEKYKEIFFDESKNGYAGYID | |
| | GGASQEEFYKYIKPILEKLDGTEYFLDKIEREDFLRKQRTFDNGSIPHQIHL | |
| | QELHAILRRQEEFYPFLKENQEKIEKILTFRIPYYVGPLARGNSRFAWLSRK | |
| | SDETITPWNFEEVVDKEASAQAFIERMTNFDKNLPEEKVLPKHSLLYEKFTV | |
| | YNELTKVKYITEGMRKPAFLSANQKEEIVDELFKKNRKVTVKKLKEFLFKEI | |
| | ECFDIVEISGVEDRFNASLGTYHDLLKIIKDKDFLDNEENEDILEDIVLTLT | |
| | LFEDREMIEQRLEKYADLFDKKVLKKLKRRRYTGWGRLSAKLINGIRDKQSG | |
| | KTILDYLIDDGFANRNFMQLIHDDSLTFKEEIKKAQVIGQGDSLHETIANLA | |
| | GSPAIKKGILQSLKIVDELVKVMGRHNPENIVIEMARENQTTQKGQKNSRER | |
| | LKRLEESIKKLGSNILKEHPVENTQLQNDKLYLYYLQNGKDMYTGEELDIDR | |
| | LSDYDVDHIIPQSFIKDDSIDNRVLTSSAKARGKSDNVPSEEVVKKMKSFWR | |
| | QLLNAKLISQRKFDNLTKAERGGLSELDKAGFIKRQLVETRQITKHVAQILD | |
| | SRFNTKYDENDKLIRKVKIITLKSKLVSQFRKDFGFYKVREINDYHHAHDAY | |
| | LNAVVGKALIKKYPKLESEFVYGDYKVYDSRKMIAKSEQEIGKATAKYFFYS | |
| | NIMNFFKSEITLANGEIRKRPLIETNEETGEIVWDKEKDFATVKKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSSFEKNPIAFLEDKGYKNIQKDSII | |
| | KLPKYSLFELENGRKRLLASAGELQKGNELALPQHYVILLYHAKRYEKLKES | |
| | PEDNEKHLEYVEQHRSEFDEILDQISEFSERYILADKNLEKIEELYEKNEDK | |
| | DISELASSFINLLTFTALGAPAAFKFFGKTIDRKRYTSTTEILNATLIHQSI | |
| | TGLYETRIDLSQLGGD | |
| CasEnd-322 | MDKPYSIGLDIGTNSVGWAVVTDEYKVPSKKFKVLGNTDRHSIKKNLLGALL | 282 |
| | FDSGETAEATRLKRTARRRYTRRKNRILYLQEIFAEEMNKVDDSFFHRLEES | |
| | FLVEEDKRFERHPIFGNIVDEVAYHEKYPTIYHLRKHLADSDEKADLRLVYL | |
| | ALAHIIKFRGHFLIEGDLNSENTDVQKLFEDFVQVYDKTFEESHLSEETVDA | |
| | SSILTAKISKSRKLENLIKQFPTEKKNGLFGNLIALSLGLQPNFKTNEDLSE | |
| | DAKLQFSKDTYEEDLENLLGQIGDDYADLFVAAKNLYDAILLSGILTVDTEI | |
| | TKAPLSASMIKRYDEHHQDLTLLKKFIRKNLPEKYKEIFFDESKNGYAGYID | |
| | GGASQEEFYKYLKPLLSKIDGSEYFLDKIEREDFLRKQRTFDNGSIPHQIHL | |
| | QELHAILRRQGEYYPFLKENQEKIEKILTFRIPYYVGPLARGNSRFAWASRK | |
| | SDETITPWNFDEVVDKEKSAEAFIERMTNNDKNLPEEKVLPKHSLLYEKFTV | |
| | YNELTKVKYVTEGMRKPAFFDANQKQEIFDGLFKKNRKVTVKKLLNFLFKEF | |
| | EEFRIVDISGVEKKFNASLGTYHDLLKIIKDKDFLDNPENEDILEDIVLTLT | |
| | LFEDREMIKKRLSKYADLFDKKQLKKLERRRYTGWGRLSAKLINGIRDKQSG | |
| | KTILDYLIDDGFANRNFMQLIHDDSLSFKEEIKKAQVIGKLDSLHEVIANLA | |
| | GSPAIKKGILQSLKIVDELVKVMGRYNPENIVIEMARENQTTQKGQRNSRER | |
| | LKRLEESLKELGSDILKEHPVENTQLQNDKLYLYYLQNGKDMYTGEELDIDR | |
| | LSDYDVDHIIPQSFIKDDSIDNRVLTSSAKARGKSDDVPSIEVVKKMKSFWS | |
| | QLLSAKLISQRKFDNLTKAERGGLTELDKAGFIKRQLVETRQITKHVAQILD | |
| | SRFNTKYDENDKLIRKVKIITLKSKLVSQFRKEFELYKVREINDYHHAHDAY | |
| | LNAVVGKALIKKYPKLESEFVYGDYKVYNSYKMIAKSEQEIGKATAKYFFYS | |
| | NIMNFFKTDITLANGEIRKRPLIETNEETGEIVWNKEKDFATVRKVLSMPQV | |
| | NIVKKTEVQTGGFSKESILPKRDSDKLIARKKDWDTKKYGGFDSPTVAYSVL | |
| | VVADIEKGKSKKLKTVKELVGITIMERSAFEKNPVAFLEDKGYKNIQKDNII | |
| | KLPKYSLFELENGRKRLLASAGELQKGNELALPQKYVILLYHAHRYEKLKES | |
| | PEDNPKHLEYVENHKSEFDEILDQISEFSKRYILADKNLEKIEELYAKNNDA | |
| | SVEELASSFINLLTFTALGAPAAFKFFGKTIDRKRYTSTTEILNATLIHQSI | |
| | TGLYETRIDLSQLGGD | |
| CasEnd-323 | MKKKYSIGLDIGTNSVGWAVITDEYKVPSKKFKVLGNTDRHSIKKNLIGALL | 283 |
| | FDSGETAEATRLKRTARRRYTRRKNRIRYLQEIFAEEMSKVDDSFFHRLEES | |
| | FLVEEDKKHERHPIFGNIVDEVAYHEKYPTIYHLRKKLADSTEKADLRLIYL | |
| | ALAHMIKFRGHFLIEGQLNPDNSDVQELFIQLLQTYNQLFEENPLKESRVDA | |
| | KAILSAKLSKSRRLENLIAQFPGEKKNGLFGNLIALSLGLTPNFKSNFDLAE | |
| | DAKLQLSKDTYDEDLDNLLAQIGDQYADLFLAAKNLSDAILLSDILTVNSES | |
| | TKAPLSASMVKRYDEHHQDLTLLKALVRQQLPEKYKEIFFDDSKNGYAGYID | |
| | GGASQEEFYKFIKPILEKMDGTEELLAKINREDFLRKQRTFDNGSIPHQIHL | |
| | GELHAILRRQEEFYPFLKENQEKIEKILTFRIPYYVGPLARGNSRFAWMTRK | |
| | SEETITPWNFEEVVDKEASAQAFIERMTNFDKNLPNEKVLPKHSLLYEYFTV | |
| | YNELTKVKYVTEGMGKPEFFSGNQKEEIVDLLFKKNRKVTVKQLKEDYFKKI | |
| | ECFDGVEISGVEDRFNASLGTYHDLLKIIKDKDFLDNEENEDILEDIVLTLT | |
| | LFEDREMIEERLEKYAHLFDKKVLKQLKRRRYTGWGRLSRKLINGIRDKQSG | |
| | KTILDFLKSDGFANRNFMQLIHDDSLTFKEEIQKAQVIGQGESLHEQIADLA | |
| | GSPAIKKGILQSVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTKRDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKSEITLANGEIRKRPLIETNEETGEIAWDKGKDFATVRKVLSMPQV | |
| | NIVKKTEVQTGGFSKESILPKRNSDKLIARKKDWDTKKYGGFDSPTVAYSVL | |
| | VVAKVEKGKVKKLKSVKELVGITIMERSSFEKNPIAFLEDKGYKEIKKDLII | |
| | KLPKYSLFELENGRKRMLASAGELQKGNELALPSHYVTLLYHASNYEKLKGK | |
| | SEDIEKKLEYVEQHRHEFDEIFEQIIEFSKRVILADANLSKVKSLFNENRDK | |
| | SIEELAENFIHLLTLTSLGAPAAFKFFDKTIDRKRYTSTTEVLNATLIHQSI | |
| | TGLYETRIDLSQLGGD | |
| CasEnd-324 | MKKPYSIGLDIGTNSVGWAVVTDDYKVPSKKMKVLGNTDRSSIKKNLLGALL | 284 |
| | FDSGETAEATRLKRTARRRYTRRKNRILYLQEIFAEEMNKVDESFFHRLEDS | |
| | FLVEEDKRGERHPIFGTIVEEVKYHEEFPTIYHLRKHLADSKEKADLRLVYL | |
| | ALAHIIKFRGHFLIEGKLDTENTDVQELFKEFLEVYDNTFERSALSEETVQV | |
| | EEILTDKISKSAKKERVLKLFPNEKSNGRFAEFLKLIVGNQADFKKHFELEE | |
| | KAKLQFSKDTYEEDLEGLLGQIGDEYADLFVSAKKLYDSILLSGILTVTDNS | |
| | TKAPLSASMVQRYEEHHEDLTKLKKFIRKKLSEKYKEVFFDKSKNGYAGYID | |
| | GGTKQEDFYKYLKKLLNKIEGSEYFLDKIEREDFLRKQRTFDNGSIPHQIHL | |
| | QEMRAIIRRQAEYYPFLAENQDKIEKILTFRIPYYVGPLARGNSRFAWASRK | |
| | SDETITPWNFDEIIDKEKSAEAFINRMTNYDLYLPDEKVLPKHSLLYEKFTV | |
| | YNELTKVKYITEQMGKTEFFDANMKQEIFDGVFKKYRKVTKDKLLNFLEKEF | |
| | DEFRIVDLSGVEKAFNASLGTYHDLKKILNDKDFLDDSENEKILEDIILTLT | |
| | LFEDREMIRKRLSKYSDLFTKEQLKKLERRHYTGWGRLSAKLINGIRDKETR | |
| | KTILDYLIDDGNSNRNFMQLIHDDALSFKEEIAKAQVIGETDSLHQVVADLA | |
| | GSPAIKKGILQSLKIVDELVKVMGRHNPENIVVEMARENQTTNKGQRNSRER | |
| | LKGLTDSIKELGSDILKEHPVDNSQLQNDRLYLYYLQNGKDMYTGEELDIDN | |
| | LSQYDVDHIIPQSFIKDDSIDNRVLVSSAKARGKSDDVPSIEVVRKMKSFWS | |
| | KLLSAKLISQRKFDNLTKAERGGLTDDDKAGFIKRQLVETRQITKHVAQILD | |
| | ERFNTETDENNKLIRKVKIVTLKSKLVSQFRKEFELYKVREINDYHHAHDAY | |
| | LNAVVGKALIGKYPQLEPEFVYGDYPKFNSFKLVRKSAKEEGKATAKKFFYS | |
| | NIMNFFKKDVKLADGTVIERPQVEVNDETGEIVWDKNKHISIVKKVLSMPQV | |
| | NIVKKTEVQTGGFSKESILPKRNSDKLIARKKKWDTKKYGGFDSPIVAYSVL | |
| | VIADIEKGKSKKLKTVKELVGITIMERSTFEKNPVAFLENKGYQNIQEENII | |
| | KLPKYSLFELEDGRKRLLASAGELQKGNELALPNHLVTLLYHAKNIEKIDEK | |
| | EEEEPEHLNYVQKHRDEFKELLDQVSEFSKRYILADKNLEKIEELYAQNNSA | |
| | DIEELASSFINLLTFTAIGAPADFKFFGKNIDRKRYTSTTEILNATLIHQSI | |
| | TGLYETRIDLSKLGED | |
| CasEnd-325 | MDKKYSIGLDIGTNSVGWAVITDEYKVPSKKFKVLGNTDRTSIKKNLLGALL | 28.5 |
| | FDSGETAEATRLKRTARRRYTRRKNRIRYLQEIFAEEMAKVDDSFFHRLEES | |
| | FLVENDKKGERHPIFGNIVDEVAYHEKYPTIYHLRKKLADSTEKADLRLIYL | |
| | ALAHMIKFRGHFLIEGDLNPENSDVQKLFIQLVQTYNQLFEESPIEEITVDA | |
| | KAILSARLSKSRRLENLIAQFPGQKKNGLFGNLIALSLGLTPNFKSNEDLSE | |
| | DAKLQLSKDTYDEDLDNLLAQIGDQYADLFLAAKNLSDAILLSDILTVNSEI | |
| | TKAPLSASMVKRYDEHHQDLTLLKALVRQQLPEKYKEIFFDDSKNGYAGYID | |
| | GGASQEEFYKYIKPILEKMDGTEELLAKLNREDFLRKQRTFDNGSIPHQIHL | |
| | GELHAILRRQEEFYPFLKENREKIEKILTFRIPYYVGPLARGNSRFAWMTRK | |
| | SEETITPWNFEEVVDKEASAQAFIERMTNFDKNLPEEKVLPKHSLLYEYFTV | |
| | YNELTKVKYVTEGMRKPEFLSGEQKEEIVDLLFKTNRKVTVKQLKEDYFKKI | |
| | ECFDSVEISGVEDRFNASLGTYHDLLKIIKDKDELDNEENEDILEDIVLTLT | |
| | LFEDREMIEQRLEKYAHLFDKKVLKQLKRRRYTGWGRLSRKLINGIRDKQSG | |
| | KTILDFLKSDGFANRNFMQLIHDDSLTFKEEIKKAQVIGQGDSLHEQIANLA | |
| | GSPAIKKGILQTLKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIRDVKIITLKSKLVSDFRKDFQFYKVREINNYHHAHDAY | |
| | LNAVVGTALIKKYPKLESEFVYGDYKVYDVRKLIAKSEQEIGKATAKYFFYS | |
| | NIMNFFKTEITLANGEIIKRPLIETNEETGEIVWNKQKDFATVRKVLSMPQV | |
| | NIVKKTEVQTGGFSKESILPKRNSDKLIARKKDWDTKKYGGFDSPTVAYSVL | |
| | VIAKIEKGKSKKLKTVKELVGITIMERSSFEKDPIGFLEKKGYKDIQKDLII | |
| | KLPKYSLFELENGRKRLLASAGELQKGNELALPSKYVTFLYLASNYEKLKGS | |
| | PEDNEQKRPYVEQHMDEFKEILDQISEFSKRYILADKNLDKIISLYNQNNDS | |
| | DIEELAENFIHLFTFTSLGAPAAFKFFDKTIDRKRYTSTTEVLNATLIHQSI | |
| | TGLYETRIDLSQLGGD | |
| CasEnd-326 | MDKKYSIGLDIGTNSVGWAVITDEYKVPSKKFKVLGNTDRHSIKKNLIGALL | 286 |
| | FDSGETAEATRLKRTARRRYTRRKNRICYLQEIFSNEMAKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVQKLFKELIDAYNQTFEESPLDEESVDA | |
| | EAILTEKLSKSRRLENLLALFPGEKKNGLFGNILALSVGLTPNFKSNEDLAE | |
| | DAKLQFSKDTYDEDLEELLGQIGDEYADLFLAAKNVYDAILLSGILTVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRLLEAIKEFKSEILKEHPVENTKLQNDKLYLYYLQNGKDMYTGEPLDIDR | |
| | LSDYDVDHIVPQSFLKDDSIDNRVLVSSEEARGKSDDVPSEAVVRKMKSYWK | |
| | KLLDAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-327 | MKKPYSIGLDIGTNSVGWAVVTDEYKVPSKKFKVLGNTDRQSIKKNLLGALL | 287 |
| | FDSGETAEATRLKRTARRRYTRRKNRILYLQEIFAEEMNKVDDSFFHRLEES | |
| | FLVEEDKRGERHPIFGNIVEEVAYHEKYPTIYHLRKHLADSTEKADLRLVYL | |
| | ALAHIIKFRGHFLIEGDLNAENTDVQKLFKKFVEVYDNTFEESHLSEETVDA | |
| | EAILTEKISKSRRLENLIAQFPNEKKNGLFGNLLALSLGLQPNFKTNFGLSE | |
| | DAKLQFSKDTYDEDLENLLGQIGDEYADLFVAAKNLYDAILLSGILTVNDSS | |
| | TKAPLSASMIKRYDEHHQDLTLLKKFIRENLPEKYKEIFFDESKNGYAGYID | |
| | GGASQEEFYKYLKKILSKVDGSEYFLEKIEREDFLRKQRTFDNGSIPHQIHL | |
| | QELHAILRRQEEYYPFLKENQEKIEKILTFRIPYYVGPLARGNSRFAWASRK | |
| | SDETITPWNFDEVVDKEASAEAFIERMTNNDKNLPEEKVLPKHSLLYEKFTV | |
| | YNELTKVKYVTEGMGKAKFFDANLKQEIFDGLFKKYRKVTKKKLLEFLFKEF | |
| | DEFRIVEISGVEKAFNASLGTYHDLLKIIKDKEFLDNPENEDILEDIVLTLT | |
| | LFEDREMIKKRLQKYADLFDKKQLKKLERRRYTGWGRLSAKLINGIRDKQSG | |
| | KTILDYLIDDGFANRNFMQLIHDDNLSFKEEIAKAQVIGESDSLHEVIADLA | |
| | GSPAIKKGILQSLKIVDELVKVMGRYAPENIVVEMARENQTTQKGQRNSRER | |
| | LKRLEEAIKELGSKILKEHPVENTQLQNDKLYLYYLQNGKDMYTGEELDIDR | |
| | LSDYDVDHIIPQSFIKDDSIDNRVLTSSAKARGKSDDVPSIEVVKKMKSEWS | |
| | KLLSAKLISQRKFDNLTKAERGGLTELDKAGFIKRQLVETRQITKHVAQILD | |
| | ERFNTEFDENDKLIRDVKIITLKSKLVSNFRKEFELYKVREINDYHHAHDAY | |
| | LNAVVGKALIKKYPKLESEFVYGDYPVYNSYKMIAKSEKEIGKATAKMFFYS | |
| | NIMNFFKTEIKLADGTVVERPVIEVNEETGEIVWDKTKHIATVKKVLSMPQV | |
| | NIVKKTEVQTGGFSKESILPKRDSDKLIARKKDWDTKKYGGFDSPTVAYSVL | |
| | VVADIEKGKSKKLKTVKELVGITIMERSRFEKNPVAFLEDKGYQNIQEEKII | |
| | KLPKYSLFELENGRKRLLASAGELQKGNELALPNKYVTLLYHAKHYEKLKEK | |
| | PEDNEKHREYVEKHRDEFDEILDQISEFSKRYILADKNLEKIKELYSKNESA | |
| | SIEELASSFINLLTFTALGAPAAFKFFGKTIDRKRYTSTKEILNATLIHQSI | |
| | TGLYETRIDLSKLGGD | |
| CasEnd-328 | MDKKYSIGLDIGTNSVGWAVITDEYKVPSKKFKVLGNTDRKSIKKNLIGALL | 288 |
| | FDSGETAEATRLKRTARRRYTRRKNRIRYLQEIFAEEMAKVDDSFFHRLEES | |
| | FLVEEDKENERHPIFGNIVDEVAYHEKYPTIYHLRKKLADSDEKADLRLVYL | |
| | ALAHMIKFRGHFLIEGDLNSDNSDVQKLFEQLVQTYNQLFEESPINEEEVDA | |
| | KAILTAKLSKSRRLENLIALFPGEKKNGLFGNLIALSLGLTPNFKSNFDLAE | |
| | DAKLQLSKDTYDEDLENLLAQIGDQYADLFLAAKNLSDAILLSGILTVKDES | |
| | TKAPLSASMVKRYDEHHQDLTLLKKLVRQQLPEKYKEIFFDQSKNGYAGYID | |
| | GGASQEEFYKYLKKILEKMDGSEEFLDKINREDFLRKQRTFDNGSIPHQIHL | |
| | QELHAILRRQEKYYPFLKDNREKIEKILTFRIPYYVGPLARGNSRFAWMSRK | |
| | SDETITPWNFEEVVDKEASAQKFIERMTNFDKNLPNEKVLPKHSLLYEYFTV | |
| | YNELTKVKYVTEGMRKPEFFSGEQKQEIVDLLFKKNRKVTVKQLKEYLFKNI | |
| | ECFDSVEISGVEDRFNASLGTYHDLLKIIKDKDELDNEENEDILEDIVLTLT | |
| | LFEDREMIEERLKKYANLFDDKVIKQLKRRRYTGWGRLSKKLINGIRDKQSG | |
| | KTILDYLKSDGFANRNFMQLIHDDSLTFKEEIEKAQVSGQGDSLHELIANLA | |
| | GSPAIKKGILQTIKIVDELVKVMGRHAPENIVIEMARENQTTQKGQKNSRER | |
| | MKRLEEAIKELGSQILKEHPVENTQLQNEKLYLYYLQNGKDMYTGEELDIDR | |
| | LSDYDVDHIVPQSFLKDDSIDNKVLVSSKKARGKSDNVPSEEVVKKMKNYWK | |
| | KLLDAKLITQRKFDNLTKAERGGLTELDKAGFIKRQLVETRQITKHVAQILD | |
| | SRFNTKYDENDKLIRDVKIITLKSKLVSQFRKDFQFYKVREINNYHHAHDAY | |
| | LNAVVGTALIKKYPKLESEFVYGDYKVYDVRKMIAKSEREIGKATAKMFFYS | |
| | NIMNFFKSEVTLANGEIRKRPLIEVNEETGEIVWDKEKDFATVRKVLSMPQV | |
| | NIVKKTEVQTGGFSKESILPKRNSDKLIARKKDWDPKKYGGFDSPTVAYSVL | |
| | VVAKVEKGKTKKLKTVKELVGITIMERSSFEKNPIAFLEAKGYKEVQEDLII | |
| | KLPKYSLFELENGRKRMLASAGELQKGNELALPSKYVTLLYLASHYEKLKGS | |
| | PEDNEEKQNYVEQHKEYFDEIIEQISEFSKRYILADANLEKIKSLYNKKRDK | |
| | SIEEQAESFINLLTFTNLGAPAAFKFFDTTIDRKRYTSTKEVLNATLIHQSI | |
| | TGLYETRIDLSKLGGD | |
| CasEnd-329 | MDKKYSIGLDIGTNSVGWAVITDEYKVPSKKFKVLGNTDRHSIKKNLIGALL | 289 |
| | FDSGETAEATRLKRTARRRYTRRKNRIRYLQEIFSEEMSKVDDSFFHRLEES | |
| | FLVEEDKKHERHPIFGNIVDEVAYHEKYPTIYHLRKKLADSTDKADLRLIYL | |
| | ALAHMIKFRGHFLIEGDLNPDNSDVDKLFIQLVQTYNQLFEENPINEEGVDA | |
| | KAILSAKLSKSRRLENLIAQLPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDPSKNGYAGYID | |
| | GGASQEEFYKFIKPILEKMDGAEELLAKLEREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKEAIVDLLFKKNRKVTVKQLKEDYFKKI | |
| | ECFDSVEISGVEDRFNASLGTYHDLLKIIKDKDFLDNEENEDILEDIVLTLT | |
| | LFEDREMIEERLKKYAHLFDDKVLKQLKRRRYTGWGRLSRKLINGIRDKQSG | |
| | KTILDFLKSDGFANRNFMQLIHDDSLTFKEEIQKAQVSGQGDSLHETIANLA | |
| | GSPAIKKGILQTIKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTEYDENDKLIREVKIITLKSKLVSDFRKDFQFYKVREINDYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKSVKELVGITIMERSSFEKDPISFLEDKGYKNVQKDLII | |
| | KLPKYSLFELENGRKRMLASAGELQKGNELALPQKYVNFLYLASHYEKLKGK | |
| | PEDNEQKLLYVEQHKHYFDEIFDQISEFSERYILADANLEKILELYNKHRDK | |
| | PISELAENFIHLFTFTSLGAPAAFKFFDTTIDRKRYTSTTEVLDATLIHQSI | |
| | TGLYETRIDLSQLGGD | |
| CasEnd-330 | MDKKYSIGLDIGTNSVGWAVITDEYKVPSKKFKVLGNTDRQSIKKNLIGALL | 290 |
| | FDSGETAEATRLKRTARRRYTRRKNRICYLQEIFSEEMSKVDDSFFHRLEES | |
| | FLVEEDKKHERHPIFGNIVDEVAYHEKYPTIYHLRKKLVDSTDKADLRLIYL | |
| | ALAHMIKFRGHFLIEGDLNPDNSDVDKLFIQLVQTYNQLFEENPINAEGVDA | |
| | KAILSARLSKSRRLENLIAQLPGEKKNGLFGNLIALSLGLTPNFKSNEDLSE | |
| | DAKLQLSKDTYDEDLDNLLAQIGDQYADLFLAAKNLSDAILLSDILRVNTEI | |
| | TKAPLSASMIKRYDEHHQDLTLLKALVRQQLPEKYKEIFFDKSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SDETITPWNFEEVVDKGASAQSFIERMTNFDKNLPNEKVLPKHSLLYEYFTV | |
| | YNELTKVKYVTEGMRKPAFLSGEQKKEIVDLLFKTNRKVTVKQLKEDLFKEI | |
| | ECFDSVEISGVEDRFNASLGTYHDLLKIIKDKDFLDNEENEDILEDIVLTLT | |
| | LFEDREMIEERLKTYANLFDKKVMKQLKRRRYTGWGRLSRKLINGIRDKQSG | |
| | KTILDFLKSDGFANRNFMQLIHDDSLTFKEDIQKAQVSGQGDSLHEQIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYTDQELDINR | |
| | LSDYDVDHIVPQSFLKDDSIDNKVLTSSKKARGKSDDVPSEEVVKKMKSYWR | |
| | QLLNAKLITQRKFDNLTKAERGGLSELDKAGFIKRQLVETRQITKHVAQILD | |
| | SRMNTKYDENDKLIRDVKVITLKSKLVSDFRKDFQFYKVREINNYHHAHDAY | |
| | LNAVVGTALIKKYPKLESEFVYGDYKVYDSRKMIAKSEQEIGKATAKYFFYS | |
| | NIMNFFKSEIKLANGEIRKRPLIETNEETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKAKKLKSVKELLGITIMERSSFEKNPVDFLEAKGYKNIQKDLII | |
| | KLPKYSLFELENGRKRLLASAGELQKGNELALPQKYVTFLYLASHYEKLKGK | |
| | PEDEEKKQLFVEQHRHYFDEILEQISEFSERYILADKNLEKILELYSEHEDY | |
| | SIREQAENIINLFTFTNLGAPAAFKYFDTTIDRKRYTSTTEILNATLIHQSI | |
| | TGLYETRIDLSQLGGD | |
| CasEnd-331 | MKKPYSIGLDIGTNSVGWAVVTDEYKVPSKKFKVLGNTDRQSIKKNLLGALL | 291 |
| | FDSGETAEATRLKRTARRRYTRRKNRILYLQEIFAEEMNKVDESFFHRLEES | |
| | FLVEEDKRGERHPIFGNIVDEVAYHEKYPTIYHLRKHLADSTEKADLRLVYL | |
| | ALAHIIKFRGHFLIEGDLNTENTDVQKLFKQFLEVYDKTFEESHLSEETVDA | |
| | EAILTEKVSKSRRLENLIAQFPNEKKNGFFGNLIALSLGLQPNFKINFELSE | |
| | DAKLQFSKDTYDEDLENLLGQIGDEYADLFVAAKNLYDAILLSGILTVNDES | |
| | TKAPLSASMIKRYDEHHQDLTLLKAFIRKNLPEKYKEIFFDKSKNGYAGYID | |
| | GGASQEEFYKYIKKLLSKIDGSEYFLEKIEREDFLRKQRTFDNGSIPHQIHL | |
| | QELKAILRRQGEYYPFLKENQEKIEKILTFRIPYYVGPLARGNSRFAWASRK | |
| | SDETITPWNFDEVVDKEASAEAFIERMTNNDKNLPNEKVLPKHSLLYEKFTV | |
| | YNELTKVKYVTEQMGKAEFFDANMKQEIFDGLFKKERKVTKKKLLDFLKKEF | |
| | DEFRIVDISGVEKAFNASLGTYHDLLKIIKDKDFLDNEENEDILEDIILTLT | |
| | LFEDREMIEKRLSKYADLFDKKVLKKLERRRYTGWGRLSAKLINGIRDKQTG | |
| | KTILDYLIDDGFANRNFMQLIHDDSLSFKEEIAKAQVIGEGDSLHEVIADLA | |
| | GSPAIKKGILQSLKIVDELVKVMGRYNPENIVVEMARENQTTQKGQRNSRER | |
| | LKRLEEAIKELGSKILKEHPVENTQLQNDKLYLYYLQNGKDMYTGEELDIDR | |
| | LSDYDVDHIIPQSFIKDDSIDNRVLTSSAKARGKSDDVPSEEVVKKMKSFWN | |
| | KLLNAKLISQRKFDNLTKAERGGLTELDKAGFIKRQLVETRQITKHVAQILD | |
| | ERFNTEFDENNKLIRDVKIVTLKSKLVSNFRKEFELYKVREINDYHHAHDAY | |
| | LNAVVGTALIKKYPKLESEFVYGDYPVYNSYKMVAKSEQEIGKATAKMFFYS | |
| | NIMNFFKTDIKLADGTIIERPVIEVNEETGEIVWDKDKHIATVKKVLSMPQV | |
| | NIVKKTEVQTGGFSKESILPKRDSDKLIARKKDWDTKKYGGFDSPTVAYSVL | |
| | VVADIEKGKSKKLKTVKELVGITIMERSRFEKNPVAFLEDKGYQNIQKDKII | |
| | KLPKYSLFELENGRKRLLASAGELQKGNELALPNKYVTLLYHAKHYEKLKEK | |
| | PEDNEKHKEYVEQHRDEFKEILDQISEFSKRYILADKNLEKIEELYSKNRNA | |
| | SIEELASSFINLLTFTALGAPAAFKFFGTTIDRKRYTSTTEILNATLIHQSI | |
| | TGLYETRIDLSKLGGD | |
| CasEnd-332 | MKKPYSIGLDIGTNSVGWAVITDEYKVPSKKFKVLGNTDRSSIKKNLLGALL | 292 |
| | FDSGETAEATRLKRTARRRYTRRKNRILYLQEIFAEEMNKVDDSFFHRLEES | |
| | FLVEEDKRGERHPIFGNIVEEVAYHEKYPTIYHLRKHLADSTEKADLRLVYL | |
| | ALAHIIKFRGHFLIEGDLNSENTDVQALFKQFLETYDSTFEESHLSEETVDA | |
| | EAILTDKVSKSRKLENLIAQFPNEKKNGFFGNLIALSLGLQPNFKTNFGLSE | |
| | DAKLQFSKDTYDEDLENLLGQIGDEYADLFVAAKNLYDAILLSGILTVNDES | |
| | TKAPLSASMIKRYDEHHQDLTLLKAFVRKQLPEKYKEIFFDKSKNGYAGYID | |
| | GGASQEEFYKYIKPLLSKVDGSEYFLEKIEREDFLRKQRTFDNGSIPHQIHL | |
| | QELKAILRRQGEFYPFLKENQEKIEKILTFRIPYYVGPLARGNSRFAWASRK | |
| | SDETITPWNFDEVVDKEASAEAFIERMTNNDKNLPEEKVLPKHSLLYEKFTV | |
| | YNELTKVKYVTEQMGKAEFFDANLKQEIFDGLFKKERKVTKKKLLEFLFKEF | |
| | DEFRIVDISGVEKAFNASLGTYHDLLKIIKDKEFLDNEENEDILEDIVLTLT | |
| | LFEDREMIKQRLSKYADLFDKKQLKKLERRRYTGWGRLSAKLINGIRDKQSG | |
| | KTILDYLIDDGFANRNFMQLIHDDSLSFKEEIAKAQVIGDSDSLHEVIADLA | |
| | GSPAIKKGILQSLKIVDELVKVMGRYNPENIVVEMARENQTTQKGQRNSRER | |
| | LKRLEEAIKELGSDILKEHPVENTQLQNDKLYLYYLQNGKDMYTGEELDIDR | |
| | LSDYDVDHIIPQSFIKDDSIDNRVLTSSAKARGKSDDVPSEEVVKKMKSFWK | |
| | KLLNAKLISQRKFDNLTKAERGGLTELDKAGFIKRQLVETRQITKHVAQILD | |
| | ERFNTEFDENNKLIRKVKIITLKSKLVSNFRKEFELYKVREINDYHHAHDAY | |
| | LNAVVGKALIKKYPKLESEFVYGDYPVYNSYKLIAKSEKEIGKATAKMFFYS | |
| | NIMNFFKTEIKLADGTVIERPQIEVNEETGEIVWDKTKHIATVRKVLSMPQV | |
| | NIVKKTEVQTGGFSKESILPKRNSDKLIARKKDWDTKKYGGFDSPTVAYSVL | |
| | VVADIEKGKKKKLKTVKELVGITIMERSRFEKNPVAFLEDKGYQNIQEENII | |
| | KLPKYSLFELENGRKRLLASAGELQKGNELALPNKYVTLLYHAKHYEKLKGK | |
| | PEDEEKHREYVEKHRSEFDEILDQISEFSKRYILADKNLEKIEELYDKNEDK | |
| | SIEELASSFINLFTFTALGAPAAFKFFGTNIDRKRYTSTKEILNATLIHQSI | |
| | TGLYETRIDLSKLGGD | |
| CasEnd-333 | MKKKYSIGLDIGTNSVGWAVITDEYKVPSKKFKVLGNTDRHSIKKNLIGALL | 293 |
| | FDSGETAEATRLKRTARRRYTRRKNRIRYLQEIFAEEMNKVDDSFFHRLEES | |
| | FLVEEDKKHERHPIFGNIVDEVAYHEKYPTIYHLRKHLVDSTEKADLRLIYL | |
| | ALAHMIKFRGHFLIEGDLNPENSDVQKLFIQLVQTYNNLFEEDPLNEEGVDA | |
| | EAILTAKLSKSRRLENLIAQFPGEKRNGLFGNLIALSLGLTPNFKSNFELSE | |
| | DAKLQLSKDTYDEDLEELLAQIGDQYADLFLAAKNLSDAILLSGILRVNDEI | |
| | TKAPLSASMIKRYDEHHQDLTLLKALVRAQLPEKYKEIFFDKTKNGYAGYID | |
| | GGASQEEFYKYIKPILEKMDGTEYLLAKLEREDLLRKQRTFDNGSIPHQIHL | |
| | GELHAILRRQEEFYPFLKENQEKIEKILTFRIPYYVGPLARGNSRFAWMKRK | |
| | SDETITPWNFEEVVDKGASAQAFIERMTNFDKNLPEEKVLPKHSLLYETFTV | |
| | YNELTKVKYVTEGMGKPEFLSAEQKKEIVDGLFKKNRKVTVKQLKEFYFKEF | |
| | DECRIVDISGVEDRFNASLGTYHDLLKIIKDKDFLDNVENEKILEDIVLTLT | |
| | LFEDREMIEKRLAKYANLFDKKVMKQLKRRRYTGWGRLSRKLINGIRDKQSG | |
| | KTILDFLKSDGFANRNFMQLIHDDSLTFKEEIDKAQVEGDGDSLHETIADLA | |
| | GSPAIKKGILQTVKVVDELVKVMGRHKPENIVIEMARENQTTQKGQKNSRER | |
| | LKRIEEGIKELGSDILKEHPVENTQLQNDKLYLYYLQNGRDMYTGEELDIDR | |
| | LSDYDVDHIVPQSFIKDDSIDNKVLTRSAEARGKSDDVPSIEVVRKMKSYWR | |
| | QLLKAGLITQRKFDNLTKAERGGLTELDKAGFIKRQLVETRQITKHVAQILD | |
| | SRMNTEHDENNKLIRDVKVITLKSKLVSDFRKDFQFYKVREINDYHHAHDAY | |
| | LNAVVGTALIKKYPKLESEFVYGDYKVYDSRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-334 | MKKPYSIGLDIGTNSVGWAVVTDDYKVPSKKMKVLGNTDRQSIKKNLLGALL | 294 |
| | FDSGETAEATRLKRTARRRYTRRKNRILYLQEIFAEEMNKVDDSFFHRLEDS | |
| | FLVEEDKRGERHPIFGNIVEEVAYHEKFPTIYHLRKHLADSTEKADLRLVYL | |
| | ALAHIIKFRGHFLIEGDLDTENTDVQKLFKQFVEVYDQTFEESHLSEETVDA | |
| | ESILTDKLSKSRRLENLLKLFPNEKKNGLFGNLIALSLGLQPNFKINFELSE | |
| | DAKLQFSKDTYDEDLENLLGQIGDEYADVFVAAKNLYDAILLSGILTVNDES | |
| | TKAPLSASMIKRYEEHHEDLTLLKKFIRKNLPEKYKEIFFDESKNGYAGYID | |
| | GGTSQEEFYKYIKKLLEKIDGSEYFLDKIEREDFLRKQRTFDNGSIPHQIHL | |
| | QELKAILRRQGEYYPFLKENQEKIEKILTFRIPYYVGPLARGNSRFAWASRK | |
| | SDETITPWNFDEVVDKEASAEAFIERMTNNDLYLPEEKVLPKHSLLYEKFTV | |
| | YNELTKVKYVTEGMGKAKFFDANMKQEIFDGLFKKNRKVTKKKLLEFLDKEF | |
| | DEFRIVDISGVEKAFNASLGTYHDLLKIIKDKEFLDNEENEDILEDIVLTLT | |
| | LFEDREMIKKRLEKYADLFDKKQLKKLERRHYTGWGRLSAKLINGIRDKQSG | |
| | KTILDYLIDDGFSNRNFMQLIHDDNLSFKEEIAKAQVIGDTDSLHEVVAELA | |
| | GSPAIKKGILQSLKIVDELVKVMGRHAPENIVVEMARENQTTAKGQRNSRER | |
| | LKRLEEAIKELGSQILKEHPVENQQLQNDRLYLYYLQNGKDMYTGEELDIDR | |
| | LSQYDVDHIIPQSFIKDDSIDNRVLTSSDKARGKSDDVPSEEVVKKMKSFWL | |
| | KLLKAKLISQRKFDNLTKAERGGLTEDDKAGFIKRQLVETRQITKHVAQILD | |
| | ERFNTEFDENNKLIRDVKIITLKSKLVSNFRKEFELYKVREINDYHHAHDAY | |
| | LNAVVGKALIKKYPKLEPEFVYGDYPKYNSYKLVAKSDKERGKATAKMFFYS | |
| | NIMNFFKTDIKLADGTIVERPVIEVNEETGEIAWDKNKHIATVKKVLSMPQV | |
| | NIVKKTEVQTGGFSKESILPKRDSDKLIARKKKWDTKKYGGFDSPTVAYSVL | |
| | VVADIEKGKAKKLKTVKELVGITIMERSRFEKNPIAFLEDKGYQNIQEENII | |
| | KLPKYSLFELENGRKRLLASAGELQKGNELALPNKLVTLLYHAKHIEKLDEK | |
| | DEDVPKHLEYVEEHRDEFKEILDQISEFSKRYILADKNLEKIEELYAKNEDA | |
| | SIEELASSFINLLTFTALGAPAAFKFFGKNIDRKRYTSTTEILNATLIHQSI | |
| | TGLYETRIDLSKLGGD | |
| CasEnd-335 | MKKPYSIGLDIGTNSVGWAVITDDYKVPSKKMKVLGNTDRKSIKKNLLGALL | 295 |
| | FDSGETAEATRLKRTARRRYTRRKNRILYLQEIFAEEMNKVDDSFFHRLEES | |
| | FLVEEDKRGERHPIFGNIVEEVAYHEKFPTIYHLRKHLADSTEKADLRLVYL | |
| | ALAHMIKFRGHFLIEGDLNTENSDVQKLFKQFVQEYNSTFEESHLEEETVDA | |
| | EEILTEKLSKSRRLENLIAQFPNEKKNGLFGNLIALMLGLQPNFKTNEDLSE | |
| | DAKLQFSKDTYDEDLENLLGQIGDEYADVFVAAKNLYDAILLSGILTVNDSS | |
| | TKAPLSASMIKRYDEHHEDLTLLKAFIRKNLPEKYKEIFFDKSKNGYAGYID | |
| | GGTSQEEFYKYIKKILEKMDGSEYFLDKIEREDFLRKQRTFDNGSIPHQIHL | |
| | QELHAILRRQGKYYPFLKENQEKIEKILTFRIPYYVGPLARGNSRFAWASRK | |
| | SDETITPWNFDEVIDKEKSAEAFIERMTNNDLYLPEEKVLPKHSLLYEKFTV | |
| | YNELTKVKYVTEGMGEAEFFDANQKQEIFDHVFKKNRKVTVKKLKNFLFKEF | |
| | DEFRIVDISGVEDAFNASLGTYHDLLKIIGDKEFLDNEENEDILEDIVLTLT | |
| | LFEDREMIEKRLSKYADLFSKKVLKKLKRRHYTGWGRLSAKLINGIRDKQSG | |
| | KTILDYLIDDGFANRNFMQLIHDDSLTFKEEIAKAQVIGNSDSLHETVADLA | |
| | GSPAIKKGILQSLKIVDELVKVMGRYNPENIVVEMARENQTTAKGQRNSRER | |
| | LKRLEEAMKELGSDILKEYPVENQQLQNDRLYLYYLQNGKDMYTGEELDIDR | |
| | LSDYDVDHIIPQSFIKDDSIDNRVLVSSAKARGKSDDVPSEEVVKKMKPEWS | |
| | KLLKAKLISQRKFDNLTKAERGGLTEDDKAGFIKRQLVETRQITKHVAQILD | |
| | SRFNTERDENDKLIRKVKIITLKSKLVSNFRKDFQFYKVREINDYHHAHDAY | |
| | LNAVVGTALIKKYPKLEPEFVYGDYPKYNSYKLIKKSEKERGKATAKMFFYS | |
| | NIMNFFKTKVKLADGTVVERPIIEVNDETGEIVWDKEKDFATVRKVLSMPQV | |
| | NIVKKTEVQTGGFSKESILPKRDSDKLIARKKKWDPKKYGGFDSPTVAYSVL | |
| | VVADIEKGKTKKLKTVKELVGITIMERSSFEKNPIAFLEAKGYQNIQENNII | |
| | KLPKYSLFELENGRKRLLASAGELQKGNELALPVKYVTLLYHAKHIEKLDGK | |
| | PEDKEKHLEYVMEHNEEFDEIWDQISEFSKRYILADKNLEKIEELYTKNNDK | |
| | PIRELASSFINLLTFTALGAPADFKFFGETIDRKRYTSTKEILNATLIHQSI | |
| | TGLYETRIDLSKLGGD | |
| CasEnd-336 | MDKPYSIGLDIGTNSVGWAVITDEYKVPSKKFKVLGNTDRHSIKKNLLGALL | 296 |
| | FDSGETAEATRLKRTARRRYTRRKNRILYLQEIFAEEMAKVDDSFFHRLEES | |
| | FLVEDDKRGERHPIFGNIVEEVAYHEKYPTIYHLRKHLADSTEKADLRLVYL | |
| | ALAHIIKFRGHFLIEGDLNTENSDVQKLFEQLLQTYDQTFEESHLSEITVDA | |
| | KAILTAKISKSRRLENLIAQIPNEKKNGLFGNLVALSLGLQPNFKSNFDLSE | |
| | DAKLQFSKDTYDEDLENLLGQIGDDYADLFVAAKNLYDAILLSGILTVNTEI | |
| | TKAPLSASMIKRYDEHHQDLTLLKDFVRENLPEKYKEIFFDKTKNGYAGYID | |
| | GGASQEDFYKYIKPILEKLDGSEYFLDKIEREDFLRKQRTFDNGSIPHQIHL | |
| | QELHAILRRQEEFYPFLKENQEKIEKILTFRIPYYVGPLARGNSRFAWMTRK | |
| | SDETITPWNFEEVVDKEASAEAFIERMTNFDKNLPDEKVLPKHSLLYEKFTV | |
| | YNELTKVKYITEQMRKPAFFDSEQKKEIVDLTFKKNRKVTKKKLKEFLDKEF | |
| | EEFRIVEISGVEDAFNASLGTYHDLLKIIKDKEFLDNEENEDILEDIVLTLT | |
| | LFEDREMIEKRLSKYADLFDKKVLKKLERRRYTGWGRLSAKLINGIRDKQSG | |
| | KTILDYLIDDGFANRNFMQLIHDDSLSFKEEIQKAQVIGKGDSLHEVIAELA | |
| | GSPAIKKGILQSLKIVDELVKVMGRYNPENIVIEMARENQTTQKGQRNSRER | |
| | LKRLEESLKKLGSKILKEHPVENTQLQNDKLYLYYLQNGKDMYTGEELDIDR | |
| | LSDYDVDHIIPQSFIKDDSIDNRVLTSSAKARGKSDDVPSEEVVKKMKSFWQ | |
| | QLLKAKLISQRKFDNLTKAERGGLTELDKAGFIKRQLVETRQITKHVAQILD | |
| | ERFNTKYDENDKLIRKVKIITLKSKLVSQFRKDFGFYKVREINDYHHAHDAY | |
| | LNAVVGKALIKKYPKLESEFVYGDYKVYNSRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VIADIEKGKNKKLKTVKELVGITIMERSSFEKDPVAFLEGKGYKNIQKDTII | |
| | KLPKYSLFELENGRKRLLASAGELQKGNELALPNKYVTLLYHASRYEKLKES | |
| | PEDNEKHLEYVEQHRSEFDEILDQISEFSKRYKLADKNLEKIQELYKDHDLF | |
| | SVEELASSFINLLTFTALGAPAAFKFFGVTIDRKRYTSTTEILDATLIHQSI | |
| | TGLYETRIDLSQLGGD | |
| CasEnd-337 | MDKKYSIGLDIGTNSVGWAVITDEYKVPSKKFKVLGNTDRHSIKKNLIGALL | 297 |
| | FDSGETAEATRLKRTARRRYTRRKNRICYLQEIFSNEMAKVDDSFFHRLEES | |
| | FLVEEDKRGERHPIFGNIVDEVAYHEKYPTIYHLRKHLADSTEKADLRLVYL | |
| | ALAHIIKFRGHFLIEGDLNSENSDVQKLFKQFVQTYDQTFEESHLSEETVDA | |
| | ESILTEKVSKSRRLENLIAQFPNEKKNGLFGNLIALSLGLQPNFKTNFELSE | |
| | DAKLQFSKDTYDEDLENLLGQIGDEYADLFLAAKNLYDAILLSGILTVNDES | |
| | TKAPLSASMIKRYDEHHQDLTLLKAFVRKQLPEKYKEIFFDESKNGYAGYID | |
| | GGASQEEFYKYIKKILEKIDGSEYFLDKIEREDFLRKQRTFDNGSIPHQIHL | |
| | QELHAILRRQEEYYPFLKENQEKIEKILTFRIPYYVGPLARGNSRFAWASRK | |
| | SDETITPWNFEEVVDKEASAEAFIERMTNFDKNLPEEKVLPKHSLLYEKFTV | |
| | YNELTKVKYVTEGMGKAEFFDANMKQEIFDGLFKKNRKVTKKKLLDFLFKEF | |
| | DEFRIVDISGVEDAFNASLGTYHDLLKIIKDKDFLDNEENEDILEDIVLTLT | |
| | LFEDREMIRKRLSKYADLFDKKQLKKLERRRYTGWGRLSAKLINGIRDKQSG | |
| | KTILDYLIDDGFANRNFMQLIHDDSLSFKEEIQKAQVIGESDSLHEHIANLA | |
| | GSPAIKKGILQTVKVVDELVKVMGRHKPENIVIEMARENQTTQKGQRNSRER | |
| | LKRLEEAIKELGSQILKEHPVENTQLQNDKLYLYYLQNGKDMYTGEELDIDR | |
| | LSDYDVDHIIPQSFIKDDSIDNRVLTSSAKARGKSDDVPSEEVVKKMKSFWS | |
| | KLLKAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKNPVAFLEDKGYQNIQEDNII | |
| | KLPKYSLFELENGRKRLLASAGELQKGNELALPAKYVTLLYHAKHYEKLKEK | |
| | PEDEEKHLEYVEKHRDEFKEILDQISEFSKRYILADKNLEKIEELYSKNENL | |
| | SIEELASSFINLLTFTALGAPAAFKFFGTTIDRKRYTSTKEILNATLIHQSI | |
| | TGLYETRIDLSKLGGD | |
| CasEnd-338 | MKKPYSIGLDIGTNSVGWAVVTDEYKVPSKKFKVLGNTDRKSIKKNLLGALL | 298 |
| | FDSGETAEATRLKRTARRRYTRRKNRIRYLQEIFAEEMNKVDESFFHRLEES | |
| | FLVEEDKRGERHPIFGNIVEEVAYHEKYPTIYHLRKHLADSPEKADLRLVYL | |
| | ALAHMIKFRGHFLIEGKLDTENTDVQKLFEHFLEVYDKTFEESRLSEITVNV | |
| | SEILTEKISKSRKLENLIKQFPTEKSNSFFGNLLALILGLQPNFKTNFSLSE | |
| | DAKLQFSKDTYDEDLEELLGQIGDDYADLFLAAKNLYDAILLSGILTVNDVS | |
| | TKAPLSASMVKRYDEHHQDLTKLKMFIREKAPAKYKEIFFDQSKNGYAGYID | |
| | GGAKQEDFYKYLKGILSKIEGSEYFLEKIEREDFLRKQRTFDNGSIPHQIHL | |
| | QELKAILRRQGVYYPFLKENQEKIEKILTFRIPYYVGPLARGNSRFAWASRK | |
| | SDETITPWNFDEVIDKEKSAEDFIERMTNNDKNLPEEKVLPKHSLLYEKFTV | |
| | YNELTKVKYVTEGMGKEEFFDANMKQEIFDGVFKKERKVTKDKLLNFLDKEF | |
| | EEFRIVDISGVEKNFNASLGTYHDLLKILNDKAFLDDKENENILEDIVLTLT | |
| | LFEDREMIRQRLQKYSDVFDKKQLKKLERRRYTGWGRLSAKLINGIRDKQSN | |
| | KTILDYLIDDGAANRNFMQLIHDDNLSFKEEIEKAQVIGESDSLHQIIADLA | |
| | GSPAIKKGILQSIKIVDELVKVMGRYNPENIVIEMARENQTTQKGQRNSRER | |
| | LKRLTESIKNLGSKILKEHPVDNTQLQNDKLYLYYLQNGRDMYTGEELDIDN | |
| | LSDYDVDHIIPQSFIKDDSIDNRVLVSSAKARGKSDDVPSIDVVRKMKSFWS | |
| | KLLKAKLISQRKFDNLTKAERGGLTELDKAGFIKRQLVETRQITKHVAQILD | |
| | ERFNTEFDENNKLIRDVKIITLKSKLVSQFRKEFELYKVREINDYHHAHDAY | |
| | LNAVVGKALIKKYPKLESEFVYGDYPVYNSYKLFKESNKEIGKATAKKFFYS | |
| | NIMNFFKSDDKLADGTIIERPQIEVNDETGEIAWKKVKHISTVKKVLSMPQV | |
| | NIVKKTEVQTGGFSKESILPKRNSDKLIARKKDWDPKKYGGFDSPTVAYSVL | |
| | VVADIEKGKAKKLKTVKELLGITIMERSAFEKNPVAFLEDKGYQNIQEDKII | |
| | KLPKYSLFELENGRKRLLASAGELQKGNELALPAKYVTLLYHAKHYEKFKEK | |
| | PEDIPKHLEYVNKHKLEFKELLNQILEFSKRYVLADKNLEKIEELYKNNKQA | |
| | SIKELATSFINLLTFTALGAPAAFKFFGNNIDRKRYTSTTEILNATLIHQSI | |
| | TGLYETRIDLSKLGED | |
| CasEnd-339 | MKKPYSIGLDIGTNSVGWAVLTDEYKVPSKKFKVLGNTDRQSIKKNLLGALL | 299 |
| | FDSGETAEATRLKRTARRRYTRRKNRILYLQEIFAEEMNKVDDSFFHRLEES | |
| | FLVEEDKRGERHPIFGNIVEEVAYHEEYPTIYHLRKHLADSTEKADLRLVYL | |
| | ALAHIIKFRGHFLIEGDLSAENTDVQKLFKKFLEVYDNTFEESHLSEETVDV | |
| | SVILTDKISKSRKLENLLAQYPNEKSNGFFGNLLKLSLGLQPNFKINFELSE | |
| | DAKLQFSKDTYEEDLENLLGQIGDDYADLFVAAKNLYDAILLSGILTVTDVS | |
| | TKAPLSASMIKRYDEHHQDLTKLKDFIRKNLPEKYKEIFFDDSKNGYAGYID | |
| | GGASQEEFYKYLKGLLSKLDGSEYFLDKIEREDFLRKQRTFDNGSIPHQIHL | |
| | QELKAILRRQGEYYPFLKENQEKIEKILTFRIPYYVGPLARGNSRFAWASRK | |
| | SDETITPWNFDEVIDKEASAEAFITRMTNYDKNLPEEKVLPKHSLLYEKFTV | |
| | YNELTKVKYVTEGMGKSEFFDANMKQEIFDGVFKKNRKVTKDKLLDFLDKEF | |
| | EEFRIVDLSGVEKRFNASLGTYHDLLKIIKDKEFLDDPENEEILEDIVLTLT | |
| | LFEDREMIRQRLSKYADLFDKKVIKKLERRRYTGWGRLSAKLINGIRDKQSG | |
| | KTILDYLIDDGRSNRNFMQLIHDDSLSFKEEIAKAQVIGETDSLHQVIADLA | |
| | GSPAIKKGILQSLKIVDELVKVMGRHNPENIVVEMARENQTTQKGQRNSRER | |
| | LKRLEDAIKELGSKILKEHPVENTQLQNDKLYLYYLQNGRDMYTGEELDIDR | |
| | LSDYDVDHIIPQSFIKDDSIDNRVLTSSAKARGKSDNVPSIEVVKKMKSFWY | |
| | KLLKAKLISQRKFDNLTKAERGGLTELDKAGFIKRQLVETRQITKHVAQILD | |
| | ERFNTEFDENNKLIRKVKIVTLKSKLVSQFRKEFEFYKVREINDYHHAHDAY | |
| | LNAVVGKALIKKYPKLESEFVYGDYPVYNSYKLVAKSDSEIGKATAKMFFYS | |
| | NIMNFFKSEIKLADGRIIERPVIERNDETGEIAWDKEKHIAIVKKVLSMPQV | |
| | NIVKKTEVQTGGFSKESILPKRNSDKLIARKKDWDTKKYGGFDSPTVAYSIL | |
| | VVADIEKGKSKKLKTVKELVGITIMERSKFEKNPVAFLERKGYQNIQEENII | |
| | KLPKYSLFELENGRKRLLASAGELQKGNELALPNHYVTLLYHAKRYEKDKEK | |
| | PEDIPKHLEYVDQHRDEFKEIFDQISEFSKRYILADKNLEKIKELYADNNEA | |
| | SIKELASSFINLLTFTALGAPAAFKFFGKNIDRKRYTSTTEILNATLIHQSI | |
| | TGLYETRIDLSKLGGD | |
| CasEnd-340 | MKKSYSIGLDIGTNSVGWAVITDEYKVPSKKFKVLGNTDRHSIKKNLIGALL | 300 |
| | FDSGETAEATRLKRTARRRYTRRKNRIRYLQEIFSSEMSKVDDSFFHRLEES | |
| | FLVEEDKRFERHPIFGNIVDEVAYHEKYPTIYHLRKKLADSPEKADLRLIYL | |
| | ALAHIIKFRGHFLIEGDLNTENTDVQKLFIQLVQTYNQLFEESHIDEEEVDA | |
| | KAILSAKLSKSRRLENLIAQLPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDDSKNGYAGYID | |
| | GGASQEEFYKYIKPILEKMDGSEYFLAKIEREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQAFIERMTNFDKNLPNEKVLPKHSLLYEKFTV | |
| | YNELTKVKYVTEGMKKPEFLSGEQKKEIVDLLFKKNRKVTVKQLKEFYFKKI | |
| | ECFDSVDISGVEDRFNASLGTYHDLLKIIKDKEFLDNEENEDILEDIVLTLT | |
| | LFEDREMIEERLRKYAHLFDKKVLKQLKRRRYTGWGRLSRKLINGIRDKQSG | |
| | KTILDFLKSDGFANRNFMQLIHDDSLTFKEEIQKAQVEGQGDSLHEQIAELA | |
| | GSPAIKKGILQSIKIVDELVKVMGRHNPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTEYDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPQIETNEETGEIVWDKEKDFATVKKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKNPVAFLEAKGYKNIQKDSII | |
| | KLPKYSLFELENGRKRLLASAGELQKGNELALPNKYVTLLYLASHYEKLKGS | |
| | PEDIELHLEYVKQHNYYFDDILDQISEFSERYILADKNLDKINSLYNENRDK | |
| | DINELAENFIHLFTFTSLGAPAAFKFFDTTIDRKRYTSTTEILNATLIHQSI | |
| | TGLYETRIDLSQLGGD | |
| CasEnd-341 | MKKPYSIGLDIGTNSVGWAVITDDYKVPAKKMKVLGNTDKSHIKKNLLGALL | 301 |
| | FDSGNTAEDRRLKRTARRRYTRRRNRLLYLQEIFSEEMSKVDESFFHRLDDS | |
| | FLVPEDKRGERHPIFGNLAEEVKYHKNFPTIYHLRKHLADSTEKADLRLVYL | |
| | ALAHIIKFRGHFLIEGDLDTENTDVQALFKDFLEVYDNTFEASHLSEQTVDA | |
| | SSILTDKISKSRKLENLLKHFPNEKKNSLFGNFLALSLGLQPNFKTNFQLSE | |
| | DAKLQFSKDTYEEDLENLLGQIGDDYADLFVAAKNLYDAILLSGILTVNDSS | |
| | TKAPLSASMVKRYEEHQKDLKELKQFIKQNLPDDYHEIFSDKTKNGYAGYID | |
| | GKTSQEEFYKYLKNILSKVEGSEYFLDKIEREDFLRKQRTFDNGSIPHQIHL | |
| | QEMHAILRRQGEYYPFLKENQEKIEKILTFRIPYYVGPLARGNSRFAWASRK | |
| | SDEKITPWNFDEVVDKESSAEAFITRMTNFDLYLPNEKVLPKHSLLYEKFTV | |
| | YNELTKVKYVTEQMGKAKFFDANMKKEIFDGLFKKNRKVTKKKLLNYLDKEF | |
| | DEFRIVDLTGLDKKFNASYGTYHDLLKILKDKEFLDDPENEDILEDIVLTLT | |
| | LFEDREMIRKRLSKYSDLFTKKQLKKLERRHYTGWGRLSAKLINGIRDKQSG | |
| | KTILDYLIDDGRSNRNFMQLINDDALSFKEEIAKAQVIGETDDLHQVVQDLA | |
| | GSPAIKKGILQSLKIVDELVKVMGNHEPENIVVEMARENQTTARGRRNSQQR | |
| | LKRLEDSIKNFGSKILKEHPVDNQQLQNDRLFLYYLQNGKDMYTGEELDINR | |
| | LSQYDIDHIIPQAFIKDDSIDNRVLTSSAKARGKSDDVPSEEVVKKMKSFWE | |
| | KLLRSGLISQRKFDNLTKAERGGLTEDDKAGFIKRQLVETRQITKHVARILD | |
| | ERFNTERDENNKRIRKVKIVTLKSNLVSQFRKEFELYKVREINDYHHAHDAY | |
| | LNAVVGKALLKKYPKLEPEFVYGEYPKYNSYKIDVRTNKEENKATAKYFFYS | |
| | NIMNMFKSTVKLADGSIIERPVIEANDETGEIAWDKTKHISTVKKVLSYPQV | |
| | NIVKKVEEQTGGFSKESILPKGDSDKLIARKTKWDTKKYGGFDSPTVAYSIL | |
| | VIADIEKGKSKKLKTVKELVGITIMEKNTFEKNPVAFLERKGYQNIQEENII | |
| | KLPKYSLFELENGRKRLLASAKELQKGNEMVLPNHLVTLLYHAKNINKSDEK | |
| | EEENPWHLSYVDKHRDEFKELLYYISNFSKKYTLAEKNLEKIEELYEQNNQE | |
| | DIKELASSFINLLTFTALGAPAAFKFFDKNIDRKRYTSTTEILNATLIHQSI | |
| | TGLYETRIDLSKLGGD | |
| CasEnd-342 | MDKKYSIGLDIGTNSVGWAVITDEYKVPSKKFKVLGNTDRQSIKKNLIGALL | 302 |
| | FDSGETAEATRLKRTARRRYTRRKNRIRYLQEIFAEEMAKVDDSFFHRLEES | |
| | FLVEEDKKHERHPIFGNIVDEVAYHEKYPTIYHLRKKLADSTDKADLRLIYL | |
| | ALAHMIKFRGHFLIEGDLNSENSDVDKLFIQLVQTYNQLFEENPINEEGVDA | |
| | KAILSAKLSKSRRLENLIAQFPGEKKNGLFGNLIALSLGLTPNFKSNFDLAE | |
| | DAKLQLSKDTYDEDLDNLLAQIGDQYADLFLAAKNLSDAILLSDILTVNSEI | |
| | TKAPLSASMIKRYDEHHQDLTLLKALVRQQLPEKYKEIFFDQSKNGYAGYID | |
| | GGASQEEFYKFIKPILEKMDGTEELLAKLNREDFLRKQRTFDNGSIPHQIHL | |
| | GELHAILRRQEDFYPFLKENREKIEKILTFRIPYYVGPLARGNSRFAWMTRK | |
| | SDETITPWNFEEVVDKEASAQAFIERMTNFDKNLPTEKVLPKHSLLYEYFTV | |
| | YNELTKVKYVTEGMRKPEFLSGEQKEAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKKYANLFDKKVLKQLKRRRYTGWGRLSRKLINGIRDKQSG | |
| | KTILDFLKSDGFANRNFMQLIHDDSLTFKEEIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNEETGEIVWDKEKDFATVRKVLSMPQV | |
| | NIVKKTEVQTGGFSKESILPKRNSDKLIARKKDWDTKKYGGFDSPTVAYSVL | |
| | VVAKVEKGKSKKLKTVKELVGITIMERSSFEKNPVDFLEAKGYKNVRKDLII | |
| | KLPKYSLFELENGRKRMLASAGELQKGNELALPQKYVTFLYLASHYEKLKGK | |
| | PEDNEQKQEYVEQHRDYFDEILEQISEFSERYILADKNLSKILELYNENEDS | |
| | SINEQAENFIHLFTFTALGAPAAFKFFDTTIDRKRYTSTTEVLDATLIHQSI | |
| | TGLYETRIDLSQLGGD | |
| CasEnd-343 | MKKKYSIGLDIGTNSVGWAVITDEYKVPSKKFKVLGNTDRQSIKKNLIGALL | 303 |
| | FDSGETAEATRLKRTARRRYTRRKNRIRYLQEIFAEEMSKVDDSFFQRLEES | |
| | FLVEEDKKHERHPIFGNIVDEVAYHEKYPTIYHLRKHLVDSTEKADLRLIYL | |
| | ALAHMIKFRGHFLIEGDLNSENSDVQKLFKQLVQTYNQLFEESAINEETVDA | |
| | SAILTAKLSKSRRLENLIAQFPGEKKNGLFGNLIALSLGLQPNFKSNENLAE | |
| | DAKLQFSKDTYEEDLENLLGQIGDQYADLFLAAKNLSDAILLSGILRANDES | |
| | TKAPLSASMIKRYDEHHQDLTLLKALVRKQLPEKYKEIFFDKTKNGYAGYID | |
| | GGASQEEFYKYIKPILEKMDGTEYLLAKLNREDLLRKQRTFDNGSIPHQIHL | |
| | GELHAILRRQEDYYPFLKENREKIEKILTFRIPYYVGPLARGNSRFAWMKRK | |
| | SNETITPWNFEEVVDKGASAQAFIERMTNFDKNLPSEKVLPKHSLLYEKFTV | |
| | YNELTKVKYVTEGMGKPEFLSSNMKKEIVDGLFKKNRKVTVKKLKEFYFKEI | |
| | ECFRIVDISGVEDRFNASLGTYHDLLKIIKDKDFLDNPENEDILEDIVLTLT | |
| | LFEDREMIEKRLKKYANLFDKEVMKKLKRRRYTGWGRLSRKLINGIRDKQSG | |
| | KTILDFLKSDGFANRNFMQLIHDDSLTFKEEIKKAQESGQGDSLHEQIANLA | |
| | GSPAIKKGILQTVKVVDELVKVMGRHKPENIVIEMARENQTTQKGQRNSRER | |
| | LKRIEEGIKELGSKILKEHPVENTQLQSDKLYLYYLQNGRDMYTGDELDIDR | |
| | LSDYDVDHIVPQSFIKDDSIDNKVLTRSKEARGKSDDVPSEEVVKKMKSYWR | |
| | QLLKAKLITQRKFDNLTKAERGGLTELDKAGFIKRQLVETRQITKHVAQILD | |
| | SRMNTERDENDKLIRDVKVITLKSKLVSDFRKDFQFYKVREINDYHHAHDAY | |
| | LNAVVGTALIKKYPKLESEFVYGDYPVYDSYKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRNSDKLIARKKDWDPKKYGGFDSPTVAYSVL | |
| | VVAKVEKGKSKKLKSVKELLGITIMERSSFEKNPIDFLEAKGYKEVKKDLII | |
| | KLPKYSLFELENGRKRMLASAGELQKGNELALPSKYVNFLYLASHYEKLKGS | |
| | PEDNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQSI | |
| | TGLYETRIDLSQLGGD | |
| CasEnd-344 | MDKKYSIGLDIGTNSVGWAVITDEYKVPSKKFKVLGNTDRHSIKKNLLGALL | 304 |
| | FDSGETAEATRLKRTARRRYTRRKNRIRYLQEIFAEEMAKVDDSFFHRLEES | |
| | FLVEEDKRGERHPIFGNIVDEVAYHENYPTIYHLRKKLADSPQKADLRLIYL | |
| | ALAHIIKFRGHFLIEGDLNAENTDVQKLFKQLVEIYDKLFEESHLSEETVDA | |
| | KSILTAKSSKSRRLENLIKQFPNEKKNGLFGNLLALSLGLQPNFKINFELAE | |
| | DAKLQFSKDTYEEDLENLLAQIGDQYADLFLAAKNLYDAILLSGILTVNTEI | |
| | TKAPLSASMVKRYDEHHQDLTLLKKLIREQLPEKYKEIFFDKSKNGYAGYID | |
| | GGASQEDFYKYLKPILSKLDGSEYFLDKIEREDFLRKQRTFDNGSIPHQIHL | |
| | GELHAILRRQEDFYPFLKENKEKIEKILTFRIPYYVGPLARGNSRFAWASRK | |
| | SDETITPWNFEEVVDQEASAEVFIERMTNYDKNLPEEKVLPKHSLLYEKFTV | |
| | YNELTKVKYITEGMRKPAFFDANQKEEIVDLLFKKNRKVTVKKLKEFLFKEI | |
| | EEFDGVDISGVEKAFNASLGTYHDLLKIIKDKDFLDNEENEDILEDIVLTLT | |
| | LFEDREMIEERLQKYAHLFDKKVLKKLKRRRYTGWGRLSAKLINGIRDKQTG | |
| | KTILDYLIDDGFANRNFMQLIHDDSLSFKEEIQKAQVIGKGDSLHEVIADLA | |
| | GSPAIKKGILQSLKIVDELVKVMGRHAPENIVIEMARENQTTQKGQKNSRER | |
| | LKRLEESIKNLGSKILKEHPVENTQLQNDKLYLYYLQNGKDMYTGEELDIDN | |
| | LSDYDVDHIIPQSFIKDDSIDNRVLTSSKKARGKSDNVPSEEVVKKMKNFWM | |
| | RLLKAKLISQRKFDNLTKAERGGLSELDKAGFIKRQLVETRQITKHVAQILD | |
| | SRFNTKYDENDKLIRKVKIITLKSKLVSDFRKDFGLYKVREINDYHHAHDAY | |
| | LNAVVGTALIKKYPKLESEFVYGDYKVYNSYRMIAKSEQEIGKATAKYFFYS | |
| | NIMNFFKKKITLANGEIRKRPLIETNDETGEIAWDKVKDIATVRKVLSMPQV | |
| | NIVKKTEVQTGGFSKESILPKRDSDKLIARKKDWDTKKYGGFDSPTVAYSVL | |
| | VVADVEKGKAKKLKTVKELVGITIMERSSFEKDPIAFLEAKGYQNIQKDTII | |
| | KLPKYSLFELENGRKRLLASAGELQKGNELALPQKYVTLLYHAKHYEKLKES | |
| | PEDNSEHKEYVEQHKDEFDEILDQVSEFSERYILADKNLEKIQELYKQNRDF | |
| | DIEELASSFINLLTFTALGAPAAFKFFDTKIDRKRYTSTTEILNATLIHQSI | |
| | TGLYETRIDLSQLGGD | |
| CasEnd-345 | MDKPYSIGLDIGTNSVGWAVVTDEYKVPSKKFKVLGNTDRKSIKKNMLGALL | 305 |
| | FDSGETAEATRLKRTARRRYTRRKNRICYLQEIFSNEMAKVDENFFHRLEES | |
| | FLVEEDKRNERHPIFGNIVEEVAYHEKYPTIYHLRKHLADSPEKADLRLVYL | |
| | ALAHIIKFRGHFLIEGDLNSENTDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEQIANLA | |
| | GSPAIKKGILQTLKIVDEIVKVMGRYAPENIVVEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | ERFNAEVDDSDKLIRDTKIITLKSKLVSDFRKDFGLYKVREINNYHHAHDAY | |
| | LNAVVGKALIKKYPKLESEFVYGDYKVFDVRKLIRKSGKEIGKATAKYFFYS | |
| | NIMNFFKSDVTLANGKLRKRPNIEVNEETGEIIWDKEKDIATIKKVLSMPQV | |
| | NIVKKTEVQTGGFSKESILPKRDSDKLIARKKDWDTSKYGGEDSPTVAYSVL | |
| | VIAKIEKGKAKKLKTVKELVGITIMERSAFEKDPVAFLEDKGYQDIQEELLI | |
| | KLPKYSLFELENGRKRLLASAGELQKGNELALPNHYVTLLYHAKHYEKLKGN | |
| | SEDNKESLNYIEEHREEFDELFDQVIEFAERYILADANIEKIKTLYEQNSEA | |
| | SLEELSENFLHLLKFTALGAPAAFKFFGADIDRKRYTSTKEILNATLIHQSI | |
| | TGLYETRIDLSKLGGD | |
| CasEnd-346 | MDKKYSIGLDIGTNSVGWAVITDEYKVPSKKFKVLGNTDRHSIKKNLIGALL | 306 |
| | FDSGETAEATRLKRTARRRYTRRKNRIRYLQEIFSEEMAKVDDSFFHRLEES | |
| | FLVEEDKKHERHPIFGNIVDEVAYHEKYPTIYHLRKKLADSTEKADLRLIYL | |
| | ALAHIIKFRGHFLIEGDLNSDNSDVDKLFIQLVQTYNQLFEENPINESGVDA | |
| | KAILSARLSKSRRLENLIAQFPGEKKNGLFGNLIALSLGLTPNFKSNEDLAE | |
| | DAKLQLSKDTYDDDLDNLLAQIGDQYADLFLAAKNLSDAILLSDILTVNSEI | |
| | TKAPLSASMIKRYDEHHQDLTLLKALVRQQLPEKYKEIFFDDSKNGYAGYID | |
| | GGASQEEFYKFIKPILEKMDGTEELLAKLNREDLLRKQRTFDNGSIPHQIHL | |
| | GELHAILRRQEDFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPEFLSGEQKEAIVDLLFKTNRKVTVKQLKEDYFKKI | |
| | ECFDSVEISGVEDRFNASLGTYHDLLKIIKDKDFLDNEENEDILEDIVLTLT | |
| | LFEDKEMIEERLEKYAHLFDKKVLKKLKRRRYTGWGRLSAKLINGIRDKQSG | |
| | KTILDFLKSDGFANRNFMQLIHDDSLTFKEAIQKAQVSGQGDSLHEQIANLA | |
| | GSPAIKKGILQTVKIVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKRDENDKLIREVKIITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEVTLANGEIRKRPLIETNEETGEIVWDKGKDFATVRKVLSMPQV | |
| | NIVKKTEVQTGGFSKESILPKRNSDKLIARKKDWDTKKYGGFDSPTVAYSVL | |
| | VVAKVEKGKSKKLKSVKELVGITIMERSSFEKDPVDFLEAKGYKNVQKDLII | |
| | KLPKYSLFELENGRKRLLASAGELQKGNELALPQKYVTLLYLASHYEKLKGS | |
| | PEDNEKKQYYVEQHRHYFDEIIEQISEFSERYILADKNLDKIKSLYKEHEDY | |
| | SISELAENFIHLFTFTALGAPAAFKFFDTTIDRKRYTSTTEILDATLIHQSI | |
| | TGLYETRIDLSQLGGD | |
| CasEnd-347 | MDKKYSIGLDIGTNSVGWAVITDEYKVPSKKFKVLGNTDRHSIKKNLIGALL | 307 |
| | FDSGETAEATRLKRTARRRYTRRKNRICYLQEIFSNEMAKVDPNFFHRLEES | |
| | FLVEEDKKHERHPIFGNIVDEVAYHEKYPTIYHLRKKLVDSTDKADLRLIYL | |
| | ALSDLIKNRGNFLIKGELPPGPLSVEELMKKLFAKYAELNPDNPVELNGVDL | |
| | SSILLARESPSSRLGRFVSQFPGVSKTSLLGQLFALILGLTPSFKSAFNLEE | |
| | DFKLSLKDDSFDDDLDYLVDLLGDKYKELFELARELHAAILYSKFYRDNPDI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTFDNGRIPYWINL | |
| | REIKAILENQEKFYPFLKENKEKILKILTFRIPYYVGELSKGDSPDSVAVRK | |
| | TNNTITPWNFEEDVDLKKSAKLYEESMRNTDPYLPGEKVLPKHSLTYQEFLL | |
| | YNELSSVKLLTPDGKEPKPLTGEEREQIINHLFLKYRKVTVEQLKEEFFKEV | |
| | YKWPEATILGVKGRFKANLETYHDLLKIIKNEEFILNEKNREILDEIVEILT | |
| | LFKDRELVEEALKKYSHLFSEKEMKRLKRRRFTGWGRYSRKLIDGLKHKKTG | |
| | KTVLDFLKDNGKNPLTFMQILHSEELDFKKILKKKTVPDKGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVVGKYKVEDVRKMFAKSEDEIGKATAKYFFYS | |
| | NIMNFFKTEITNENGGIEKRDPTSTNGETGEISWDKGRDFATVRKVLSMPQV | |
| | NIVKKTEVQTGGFSKESILPKRNSDKLIARKKDWDPKKYGGFDSPTVAYSVL | |
| | VVAKVEKGKSKELKSVKELLGITIMERSSFEKNPLDFLKAKGYTDVDKDKLI | |
| | YLPKYSLFELGNGRKVLLASAGELQKGNELALPFKYQEFLYLAAHLDDLKGK | |
| | PEEQEQKQLFVEQNKHYLDEIMEQISEFSKRVVNAGAQLDKVKAAWEKHKDK | |
| | PIREQAENIIHLFTLTNLGAPAAFKYFDTTIDRKRYTSTKEVLDATLIHQSI | |
| | TGLYETRIDLKQLGGD | |
| CasEnd-348 | MKKKYSIGLDIGTNSVGWAVITDEYKVPSKKFKVLGNTDRHSIKKNLIGALL | 308 |
| | FDSGETAEATRLKRTARRRYTRRKNRICYLQEIFSSEMSKVDDSFFHRLEES | |
| | FLVEEDKRNERHPIFGNIVDEVAYHEKYPTIYHLRKKLADSTEKADLRLIYL | |
| | ALAHMIKFRGHFLIEGDLNAENSDVDKLFIQLVQTYNQLFEENPIEEELVDA | |
| | KAILSAKLSKSRRLENLIAQLPGEKKNGLFGNLLALSLGLTPNFKSNEDLSE | |
| | DAKLQLSKDTYDDDLDNLLAQIGDQYADLFLAAKNLSDAILLSDILTVNGES | |
| | TKAPLSASMIKRYDEHHQDLTLLKTLVRQQLPEKYKEIFFDDSKNGYAGYID | |
| | GGASQEEFYKYIKPILEKMDGTEEFLAKLEREDFLRKQRTFDNGSIPHQIHL | |
| | GELHAILRRQEDFYPFLKENREKIEKILTFRIPYYVGPLARGNSRFAWMTRK | |
| | SEETITPWNFEEVVDKEASAQSFIERMTNFDKNLPKEKVLPKHSLLYEYFTV | |
| | YNELTKVKYVTEGMRKPEFLSAGQKKAIVDLLFKTNRKVTVKQLKEDYFKKI | |
| | ECFDTVEISGVEDRFNASLGTYHDLLKIIKDKDFLDNEENEAILEDIVLTLT | |
| | LFEDREMIEERLAKYADLFDKKVLKQLKRRRYTGWGRLSRKLINGIRDKQSG | |
| | KTILDFLKDDGFANRNFMQLIHDDSLTFKEEIQKAQVIGKGDSLHEQIANLA | |
| | GSPAIKKGILQSIKIVDELVKVMGRHAPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRFNTERDENDKLIRRVKIITLKSKLVSDFRKDFQFYKVREINDYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNTETGEIVWDKGKDFATVRKVLSMPQV | |
| | NIVKKTEVQTGGFSKESILPKRESDKLIARKKDWDTKKYGGFDSPTVAYSVL | |
| | VVAKVEKGKTKKLKTVKELVGITIMERSSFEKDPVSFLEAKGYQNIQKDLII | |
| | KLPKYSLFELENGRKRLLASAGELQKGNELALPQKYVILLYHASHYEKLKGK | |
| | EEDNSQHREYVEQHRYEFDEILDQIIEFSERYILADKNLEKILELYNENEAA | |
| | DIEELAENFIHLFTFTALGAPAAFKFFDTTIDRKRYTSTTEILDATLIHQSI | |
| | TGLYETRIDLSQLGGD | |
| CasEnd-349 | MDKPYSIGLDIGTNSVGWAVVTDEYKVPSKKFKVLGNTDRHSIKKNLLGALL | 309 |
| | FDSGETAEATRLKRTARRRYTRRKNRIRYLQEIFAEEMNKVDDSFFHRLEES | |
| | FLVEEDKRGERHPIFGNIVEEVAYHEEYPTIYHLRKHLADSPEKADLRLVYL | |
| | ALAHIIKFRGHFLIEGELDTENTDIQRLFKEFLAVYDNTFEESHLSEQNVQA | |
| | EEILTDKISKSAKKERVLKLFPNEKSNGFFAEFLKLIVGNQADFKKHFELSE | |
| | KAPLQFSKDTYEEDLENLLGQIGDDYADLFVSAKKLYDSILLSGILTVTTEI | |
| | TKAPLSASMVKRYDEHHQDLTKLKQFIRENLPDKYKEIFFDKSKNGYAGYID | |
| | GGATQEDFYKYLKGLLNKIEGSEYFLDKIEREDFLRKQRTFDNGSIPHQIHL | |
| | QELRAIIRRQGEYYPFLKENQDKIEKILTFRIPYYVGPLARGNSRFAWLSRK | |
| | SDETITPWNFDEIVDKESSAEAFINRMTNYDKNLPEEKVLPKHSLLYEKFTV | |
| | YNELTKVKYITEQMRKPAFFDANMKQEIFDGVFKVYRKVTKDKLLDFLEKEF | |
| | DEFRIVDLSGVEKAFNASLGTYHDLKKILKDKDFLDNSKNEKILEDIVLTLT | |
| | LFEDREMIRKRLSKYADLLTKEQLKKLERRRYTGWGRLSAKLINGIRDKETG | |
| | KTILDYLIDDGFSNRNFMQLIHDDSLSFKEEIAKAQVIGETDSLHQVIADLA | |
| | GSPAIKKGILQSLKIVDELVKVMGRHAPENIVVEMARENQTTQKGQRNSRER | |
| | LKRLTDSIKELGSNILKEHPVDNTQLQNDKLYLYYLQNGKDMYTGEELDIDK | |
| | LSDYDVDHIIPQSFIKDDSIDNRVLVSSAKARGKSDDVPSIEVVRKMKSFWS | |
| | QLLDAKLISQRKFDNLTKAERGGLTELDKAGFIKRQLVETRQITKHVAQILD | |
| | ERFNTKYDENDKLIRDVKIVTLKSKLVSNFRKEFELYKVREINDYHHAHDAY | |
| | LNAVVGKALIKKYPKLESEFVYGDYPVFNSYKMIAKSEQEIGKATAKYFFYS | |
| | NLMNFFKSDVTLANGEIRKRPLVETNDENGEIIWDKTKHISTVKKVLSMPQV | |
| | NIVKKTEVQTGGFSKESILPKRNSDKLIARKKDWDTKKYGGFDSPTVAYSVL | |
| | VIADIEKGKAKKLKTVKELVGITIMERSAFERDPVAFLENKGYQNIRKENII | |
| | KLPKYSLFELENGRKRLLASAGELQKGNELALPAHYVTLLYHAKNYEKIKES | |
| | PEDNPKHLEYVVKHRDEFKELLDQISEFSKRYILADKNLEKIEELYAQNEEA | |
| | DIEELASSFINLLTFTALGAPAAFKFFGKKIDRKRYTSTTEILNATLIHQSI | |
| | TGLYETRIDLSQLGGD | |
| CasEnd-350 | MDKKYSIGLDIGTNSVGWAVITDEYKVPSKKFKVLGNTDRHSIKKNLIGALL | 310 |
| | FDSGETAEATRLKRTARRRYTRRKNRICYLQEIFSNEMAKVDESFFHRLEES | |
| | FLVEEDKQGERHPIFGNIVDEVAYHEKYPTIYHLRKHLADSTEKADLRLVYL | |
| | ALAHIIKFRGHFLIEGDLNSENTDVQKLFKQFVQVYNRLFEESHLNEETVDA | |
| | ESILTEKISKSRRLENLIAQFPNEKKNGLFGNLIALSLGLQPNFKSNFELSE | |
| | DAKLQLSKDTYEEDLEELLGQIGDEYADLFVAAKNLYDAILLSGILTVNDES | |
| | TKAPLSASMIKRYDEHHQDLTLLKEFVRQNLPEKYKEIFFDKTKNGYAGYID | |
| | GGASQEEFYKYIKPILEKIDGSEYFLEKIEREDFLRKQRTFDNGSIPHQIHL | |
| | QELHAILRRQEKYYPFLKENQEKIEKILTFRIPYYVGPLARGNSRFAWVTRK | |
| | SDETITPWNFEEVVDKEKSAERFIERMTNNDKNLPDEKVLPKHSLLYEKFTV | |
| | YNELTKVKYVTEGMGKAKFFDANMKQEIFDGLFKKHRKVTKKKLLDELDKEF | |
| | EEFRIVDISGVEDAFNASLGTYHDLLKIIKDKEFLDNPENEDILEDIVLTLT | |
| | LFEDREMIEKRLQKYADLFTKKQLKKLKRRRYTGWGRLSKKLINGIRDKQSG | |
| | KTILDYLIDDGYSNRNFMQLIHDDGLSFKEEIKKAQVTGDSDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVAKIEKGKTSKLKTVKELVGITIMERSRFEKNPVKFLEAKGYQNIRKDKII | |
| | KLPKYSLFELENGRKRLLASAGELQKGNELALPKKYVTLLYHAKHYEKLKEK | |
| | SEDEEKHLNYVQKHLSEFDEIFDQISEFSKRYVLADKNLEKIEELYSQIESK | |
| | SISELAESFINLLTFTALGAPAAFKFLGLTIDRKRYTSTTEILSATLIHQSI | |
| | TGLYETRIDLSKLGGD | |
| CasEnd-351 | MDKPYSIGLDIGTNSVGWAVITDEYKVPSKKFKVLGNTDRHSIKKNLWGALL | 311 |
| | FDSGETAEATRLKRTARRRYTRRKNRIRYLQEIFSEEMSKVDDSFFQRLEES | |
| | FLVEEDKRHERHPIFGNIVDEVAYHEKYPTIYHLRKHLADSTEKADLRLIYL | |
| | ALAHIIKFRGHFLIEGDLNAENTDVQKLFIQLVQTYNQTFEEDHISEQGVDA | |
| | EAILTAKTSKSRRLENLIKQFPGEKKNGLFGNLIALSLGLQPNFKTNFDLPE | |
| | DAKLQFSKDTYDEDLENLLAQIGDQYADLFLAAKNLYDAILLSGILTVKTEI | |
| | TKAPLSASMIKRYDEHHQDLTLLKAFIREQLPEKYKEIFFDKSKNGYAGYID | |
| | GGASQEEFYKYIKPILSKIEGAEYFLDKIEREDFLRKQRTFDNGSIPHQIHL | |
| | KELKAILRRQGEFYPFLKENKEKIEKILTFRIPYYVGPLARGNSRFAWMSRK | |
| | SEETITPWNFEEVVDKEASAEAFIERMTNFDKNLPEEKVLPKHSLLYEKFTV | |
| | YNELTKVKYVTEGMRKPAFFSGEQKKEIVDELFKKNRKVTVKQLLEHLFKEF | |
| | DEFDSVEISGVEDQFNASLGTYHDLLKIIKDKEFLDNEENEDILEDIVLTLT | |
| | LFEDREMIKQRLSKYADLFDKKVLKKLKRRRYTGWGRLSAKLINGIRDKQTG | |
| | KTILDYLIDDGFANRNFMQLIHDDSLSFKEEIAKAQVIGKTDSLHEVIANLA | |
| | GSPAIKKGILQSIKIVDELVKVMGRHNPENIVIEMARENQTTQKGQRNSRER | |
| | LKRLEEVIKKLGSQILKEHPVENTQLQNDKLYLYYLQNGKDMYTGEELDIDR | |
| | LSDYDVDHIIPQSFIKDDSIDNRVLTSSDKARGKSDNVPSIEVVKKMKSYWQ | |
| | QLLNSKLISQRKFDNLTKAERGGLSELDKAGFIKRQLVETRQITKHVAQILD | |
| | SRFNTKYDENDKLIRRVKIITLKSKLVSDFRKDFGFYKVREINDYHHAHDAY | |
| | LNAVVGTALIKKYPKLESEFVYGDYKVYDSRKMIAKSEQEIGKATAKYFFYS | |
| | NIMNFFKKEITLANGEIRKRPLIETNEETGEIVWDKTKDFATVRKVLSMPQV | |
| | NIVKKTEVQTGGFSKESILPKRNSDKLIARKKDWDTKKYGGFDSPTVAYSVL | |
| | VVADVEKGKAKKLKTVKELVGITIMERSSFEKDPVLFLESKGYKNIQKDKII | |
| | KLPKYSLFELENGRKRLLASAGELQKGNELALPQKYVTLLYHAKKYEKLKGS | |
| | PEDNPKHLEYVEEHRDEFDEILDQISEFSKRYILADANLEKIKELYRKNADS | |
| | SISELASSFINLFTFTALGAPAAFKFFDEDIDRKRYTSTTEILNATLIHQSI | |
| | TGLYETRIDLSQLGGD | |
| CasEnd-352 | MDKKYSIGLDIGTNSVGWAVITDEYKVPSKKFKVLGNTDRHSIKKNLIGALL | 312 |
| | FDSGETAEATRLKRTARRRYTRRKNRICYLQEIFSNEMAKVDDSFFHRLEES | |
| | FLVEEDKRGERHPIFGNIVDEVAYHEKYPTIYHLRKHLADSTEKADLRLIYL | |
| | ALAHMIKFRGHFLIEGDLNSENTDVQKLFKKFVQVYNQTFEESALSEIGVDA | |
| | KSILTAKVSKSRRLENLIKLYPNEKKNGLFGNLIALSLGLQPNFKKNENLSE | |
| | DAKLQFSKDTYDEDLENLLGQIGDEYADLFVAAKNLYDAILLSGILTVNDES | |
| | TKAPLSASMVKRYDEHHQDLTLLKHFVRKQLPEKYKEIFFDKSKNGYAGYID | |
| | GGASQEDFYKYIKPILEKQDGTEYLLDKIEREDFLRKQRTFDNGSIPHQIHL | |
| | QELHAILRRQEEYYPFLKENQEKIEKILTFRIPYYVGPLARGNSRFAWMSRK | |
| | SDETITPWNFEEVVDKEASAEAFIERMTNFDKNLPEEKVLPKHSLLYEKFTV | |
| | YNELTKVKYVTEQMGKAEFFDSNQKEEIFDGLFKKERKVTKKKLLDFLFKEF | |
| | EEFRIVDLSGVEDAFNASLGTYHDLLKIIKDKDFLDDEENEDILEDIILTLT | |
| | LFEDREMIEKRLQKYADLFTKDQLKKLERRRYTGWGRLSKKLINGIRDKQSG | |
| | KTILDYLIDDGFTNRNFMQLIHDDSLSFKEEIAKAQVKGDEDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDTKKYGGFDSPTVAYSVL | |
| | VVADIEKGKNKKLKTVKELVGITIMERSSFEKDPVDFLEKKGYQNIQEELII | |
| | KLPKYSLFELENGRKRLLASAGELQKGNELALPNKYVTLLYHAHHYEKSKEK | |
| | PEDNEKHLKYVEKHKNEFDEILDQIEEFSKRYVLADKNLEKIVALYSKNENA | |
| | SIEELASSFINLLTFTALGAPAAFKFFGLKIDRKRYTSTTEILNSTLIHQSI | |
| | TGLYETRIDLSKLGGD | |
| CasEnd-353 | MDKKYSIGLDIGTNSVGWAVVTDDYKVPSKKFKVLGNTDRKSIKKNLLGALL | 313 |
| | FDSGETAEATRLKRTARRRYTRRKNRICYLQEIFSNEMAKVDESFFHRLEES | |
| | FLVEEDKRNERHPIFGNIVEEVAYHEKFPTIYHLRKKLADSDEKADLRLVYL | |
| | ALAHIIKFRGHFLIEGDLNAENTDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVKDES | |
| | TKAPLSASMVKRYEEHHKDLTLLKNFIRKQLPEKYKEIFFDESKNGYAGYID | |
| | GGASQEEFYKYLKKILEKIDGSEEFLDKIEREDFLRKQRTFDNGSIPHQIHL | |
| | QELHAILRRQEKYYPFLKENQEKIEQILTFRIPYYVGPLARGNSRFAWLSRK | |
| | SDETITPWNFDEVVDKEASAEAFIERMTNFDKNLPDEKVLPKHSLLYEKFTV | |
| | YNELTKVKYVTEGGKKPEFFSANQKQEIFDNVFKKNRKVTKKQLLDFLKKEF | |
| | DEFRIVDISGVEDRFNASLGTYHDLLKIIKDKDFLDNEENEEILEDIILTLT | |
| | LFEDREMIKERLEKYADLFDKEQLKKLERRRYTGWGRLSAKLINGIRDKQTG | |
| | KTILDYLIDDGFANRNFMQLIHDDSLTFKEEIAKAQVIGETDSLHELIANLA | |
| | GSPAIKKGILQSLKIVDELVKVMGRYAPENIVVEMARENQTTAKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLTDDDKAGFIKRQLVETRQITKHVAQILD | |
| | SRFNTEYDENGKLIRDVKIITLKSKLVSQFRKDFELYKVREINDYHHAHDAY | |
| | LNAVVGTALIKKYPKLESEFVYGDYKKYNLRKLIAKSDKEIGKATAKMFFYS | |
| | NIMNFFKTDVKLADGEIRKRPLIEVNEETGEIAWDKEKHIATVKKVLSMPQV | |
| | NIVKKTEVQTGGFSKESILPKRDSDKLIARKKDWDTKKYGGFDSPTVAYSVL | |
| | VVADIEKGKAKKLKTVKELVGITIMERSAFEKNPVAFLEDKGYQNIQEDNLI | |
| | KLPKYSLFELENGRKRLLASAGELQKGNELALPNHLVTLLYHAKHIEKLDGK | |
| | PEDNKEKLNYVEEHREEFDEILDQVIEFAKRYILADANIEKIKKLYEKNRSA | |
| | DIEELASSFINLLTFTALGAPAAFKFFGKTIDRKRYTSTTEILNATLIHQSI | |
| | TGLYETRIDLSKLGGD | |
| CasEnd-354 | MDKKYSIGLDIGTNSVGWAVITDEYKVPSKKFKVLGNTDRHSIKKNLIGALL | 314 |
| | FDSGETAEATRLKRTARRRYTRRKNRICYLQEIFSNEMAKVDPSFFKRLEES | |
| | FLVEEDKSTSRHPIFGNIVEEVAYHEKYPTIYHLRKKLVDSDEKADLRLIYL | |
| | ALAHMIKFRGHFLIEGDLNPDNSDVDKLFIQLVQTYNQLFEENPINASGVDA | |
| | KAILSARLSKSRRLENLIAQLPGEKKNGLFGNLIALSLGLTPNFKSNFDLAE | |
| | DAKLQLSKDTYDDDLDNLLAQIGDQYADLFLAAKNLSDAILLSDILRVDTTD | |
| | TRAPLSASMIKRYDDHHQDLTLLKELVRKYLPEKYKEIFFNQNANGYAGYID | |
| | GGATQEEFYKYIKPILESMPGTKELLEKLENKDLLRKQRTFDNGSIPHQIHL | |
| | GELRAILERQEKFYPFLKENREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDFLDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRDSDKLIARKKDWDPKKYGGFDSPTVAYSVL | |
| | VVAKKEVGKNKELKEVKDLLGITIMERSEFEKDPIGFLKKKGYVDVKEDEII | |
| | KLPKYSLFELGNGRKRMLASAGELQKGNELALPSEYVNFLYLASDYEKLKGK | |
| | EEEKKEKQKYVEENKEYLDKIIEQISEFSRRVIGADANLEKVLEAYKKHKDK | |
| | PIKEQAENIIHLFTLTALGAPAAFKYFDETIDRKRYTSTKEVLDATLIHQSI | |
| | TGLYETRIDLKFLGGD | |
| CasEnd-355 | MDKKYSIGLDIGTNSVGWAVITDEYKVPSKKFKVLGNTDRHSIKKNLIGALL | 315 |
| | FDSGETAEATRLKRTARRRYTRRKNRICYLQEIFSNEMAKVDDSFFHRLEES | |
| | FLVEEDKRGERHPIFGNIVEEVAYHEKYPTIYHLRKHLADSTEKADLRLVYL | |
| | ALAHMIKFRGHFLIEGDLNSENSDVQKLFEQFVETYDQLFEESPLSEETVDA | |
| | KAILTAKLSKSRRLENLIKQFPNEKKNGLFGNLIALSLGLQPNFKSNFELSE | |
| | DAKLQFSKDTYDEDLENLLGQIGDEYADLFLAAKNLYDAILLSGILTVNDES | |
| | TKAPLSASMIKRYDEHHQDLTLLKAFIRKQLPEKYKEIFFDESKNGYAGYID | |
| | GGASQEEFYKYIKPILSKMDGSEYFLEKIEREDFLRKQRTFDNGSIPHQIHL | |
| | QELHAILRRQEEYYPFLKENQEKIEKILTFRIPYYVGPLARGNSRFAWASRK | |
| | SDETITPWNFDEVVDKEASAQAFIERMTNFDKNLPEEKVLPKHSLLYEKFTV | |
| | YNELTKVKYVTEGMGKPQFFDANQKQEIVDLLFKKNRKVTKKKLLEFLFKEF | |
| | EEFRIVDISGVEDRFNASLGTYHDLLKIIKDKDFLDNEENEDILEDIVLTLT | |
| | LFEDREMIEKRLKKYANLFDKKQLKKLKRRRYTGWGRLSKKLINGIRDKQSG | |
| | KTILDYLIDDGFANRNFMQLIHDDSLTFKEEIKKAQVIGDSDSLHEHIANLA | |
| | GSPAIKKGILQTVKVVDELVKVMGRHKPENIVIEMARENQTTQKGQRNSRER | |
| | LKRLEEAIKELGSQILKEHPVENTQLQNDKLYLYYLQNGKDMYTGEELDIDR | |
| | LSDYDVDHIIPQSFIKDDSIDNRVLTSSAKARGKSDNVPSEEVVKKMKSYWR | |
| | RLLNAKLISQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRKSDKLIARKKDWDTKKYGGFDSPTVAYSVL | |
| | VVADVEKGKAKKLKTVKELVGITIMERSSFEKNPIAFLEKKGYQNIQEDNII | |
| | KLPKYSLFELENGRKRLLASAGELQKGNELALPAKYVILLYHAKNYEKLKEK | |
| | PEDEEKHLEYVEQHRDEFDEILDQIVEFSERYILADKNLEKIEELYSKNESK | |
| | SIEELASSFINLLTLTALGAPAAFKFLGTDIDRKRYTSTKEILNATLIHQSI | |
| | TGLYETRIDLSKLGGD | |
| CasEnd-356 | MKKPYSIGLDIGTNSVGWAVVTDDYKVPSKKMKVLGNTDRQSIKKNLLGALL | 316 |
| | FDSGETAEATRLKRTARRRYTRRKNRIRYLQEIFAEEMNKVDDSFFHRLEDS | |
| | FLVEEDKRGERHPIFGNIVEEVAYHEKFPTIYHLRKHLADSTEKADLRLVYL | |
| | ALAHIIKFRGHFLIEGDLNTENTDVQKLFKDFLQVYDQTFEDSHLSEETVDA | |
| | ESILTEKISKSRRLENLIKQFPNEKKNGLFGNLIALSLGLQPNFKINFELSE | |
| | DAKLQFSKDTYEEDLENLLGQIGDEYADLFLAAKNLYDAILLSGILTVDDSS | |
| | TKAPLSASMIKRYEEHHEDLTKLKKFIRQNLPEKYKEIFFDESKNGYAGYID | |
| | GGTKQEEFYKYLKNLLSKIDGSEYFLDKIEREDFLRKQRTFDNGSIPHQIHL | |
| | QELHAILRRQEKFYPFLKENQEKIEKILTFRIPYYVGPLARGNSRFAWMSRK | |
| | SDETITPWNFDEVVDKEASAEAFIERMTNNDLYLPNEKVLPKHSLLYEKFTV | |
| | YNELTKVKYVTEGMGKAEFFDANMKQEIFDGLFKKYRKVTKKKLLNFLFKEF | |
| | DEFRIVDISGVEKTFNASLGTYHDLLKILKDKDFLDNEENEKILEDIVLTLT | |
| | LFEDREMIKKRLEKYADLFDKKQLKKLERRHYTGWGRLSAKLINGIRDKQSG | |
| | KTILDYLIDDGNANRNFMQLIHDDNLSFKEEIAKAQVIGETDSLHEIVADLA | |
| | GSPAIKKGILQSLKIVDELVKVMGRHNPENIVVEMARENQTTAKGQRNSRER | |
| | LKRLEEAIKELGSQILKEHPVENSQLQNDRLYLYYLQNGKDMYTGEELDIDK | |
| | LSQYDVDHIIPQSFIKDDSIDNRVLTSSAKARGKSDDVPSEEVVKKMKSFWS | |
| | KLLSAKLISQRKFDNLTKAERGGLTEDDKAGFIKRQLVETRQITKHVAQILD | |
| | ERFNTEFDENNKLIRDVKIITLKSKLVSQFRKEFELYKVREINDYHHAHDAY | |
| | LNAVVGKALIKKYPKLEPEFVYGDYPKYNSYKMIAKSDQERGKATAKMFFYS | |
| | NIMNFFKSDVKLADGTIVVRPQIEVNEETGEIVWDKTKHIATVRKVLSMPQV | |
| | NIVKKTEVQTGGFSKESILPKRESDKLIARKKKWDTKKYGGFDSPTVAYSVL | |
| | VVADIEKGKAKKLKTVKELVGITIMERSRFEKNPVAFLEDKGYQNIQKENII | |
| | KLPKYSLFELENGRKRLLASAGELQKGNELALPAKLVTLLYHAKHIEKLKEK | |
| | PEDKPKHLEYVEEHRDEFKELLDQISEFSKRYILADKNLEKIEELYAKNENA | |
| | SIEELASSFINLLTFTALGAPADFKFFGETIDRKRYTSTKEILNATLIHQSI | |
| | TGLYETRIDLSKLGGD | |
| CasEnd-357 | MDKPYSIGLDIGTNSVGWAVVTDEYKVPSKKFKVLGNTDRKSIKKNLLGALL | 317 |
| | FDSGETAEATRLKRTARRRYTRRKNRICYLQEIFSNEMAKVDDNFFHRLEES | |
| | FLVEEDKRGERHPIFGNIVDEVAYHEKYPTIYHLRKELADSDEKADLRLVYL | |
| | ALAHIIKFRGHFLIEGDLNSENTDVDKLFIQLVQTYNQLFEENPINASGVDA | |
| | KAILSARLSKSRRLENLIAQLPGEKKNGLFGNLIALSLGLTPNFKSNEDLAE | |
| | DAKLQLSKDTYDDDLDNLLAQIGDQYADLFLAAKNLSDAILLSDILRVNTEI | |
| | TKAPLSASMIKRYDEHHQDLTLLKALVRQQLPEKYKEIFFDQSKNGYAGYID | |
| | GGASQEEFYKFIKPILEKMDGTEELLVKLNREDLLRKQRTEDNGSIPHQIHL | |
| | GELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTV | |
| | YNELTKVKYVTEGMRKPAFLSGEQKKAIVDLLFKTNRKVTVKQLKEDYFKKI | |
| | ECFDSVEISGVEDRFNASLGTYHDLLKIIKDKDELDNEENEDILEDIVLTLT | |
| | LFEDREMIEERLKTYAHLFDDKVMKQLKRRRYTGWGRLSRKLINGIRDKQSG | |
| | KTILDFLKSDGFANRNFMQLIHDDSLTFKEDIQKAQVSGQGESLHEQIANLA | |
| | GSPAIKKGILQSLKIVDEIVKVMGRYAPENIVVEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | ERFNAEVDDNGKLIRDVKIVTLKSKLVSDFRKDFELYKVREINNYHHAHDAY | |
| | LNAVVGKALIKKYPKLESEFVYGDYKVFDVRKLIGKSDKEIGKATAKYFFYS | |
| | NIMNFFKSDVTLANGTVRKRPIIEVNEETGEIVWDKEKHIATVKKVLSMPQV | |
| | NIVKKTEVQTGGFSKESILPKRDSDKLIARKKDWDTSKYGGFDSPTVAYSVL | |
| | VIADVEKGKAKKLKTVKELVGITIMERSAFEKDPVAFLEDKGYQDIQEILLI | |
| | KLPKYSLFELENGRKRLLASAGELQKGNELALPNHYVTLLYHAKNYEKIKGS | |
| | EEDEKESEIYIEKHREEFDEIFDQIIEFAERYILADANIEKLKELFEKNENA | |
| | SLEELSENFLHLLTFTAFGAPAAFKFFGKDIDRKRYTSPKEILNSTLIHQSI | |
| | TGLYETRIDLSKLGGD | |
| CasEnd-358 | MDKKYSIGLDIGTNSVGWAVITDEYKVPSKKFKVLGNTDRHSIKKNLIGALL | 318 |
| | FDSGETAEATRLKRTARRRYTRRKNRICYLQEIFSNEMAKVDDSFFHRLEES | |
| | FLVEEDKRGERHPIFGNIVDEVAYHEEYPTIYHLRKKLADSTEKADLRLIYL | |
| | ALAHMIKFRGHFLIEGDLNAENTDVQKLFKQLVQVYNKTFEESPLSEITVDA | |
| | KAILTEKLSKSRRLENLIKLFPNEKKNGLFGNLIALSLGLQPNFKKNFELSE | |
| | DAKLQFSKDTYDEDLENLLGQIGDQYADLFLAAKNLYDAILLSGILTVNDES | |
| | TKAPLSASMIKRYDEHHQDLTLLKNFVRQQLPEKYKEIFFDESKNGYAGYID | |
| | GGASQEEFYKYIKPILEKIDGSEYFLDKIEREDFLRKQRTFDNGSIPHQIHL | |
| | GELHAILRRQEEYYPFLKENQEKIEKILTFRIPYYVGPLARGNSRFAWATRK | |
| | SDETITPWNFEEVVDKEASAQAFIERMTNFDKNLPEEKVLPKHSLLYEKFTV | |
| | YNELTKVKYVTEGMGKPEFFDAEQKQEIFDLLFKKYRKVTVKKLLDFLFKEF | |
| | DEFRIVDISGVEDAFNASLGTYHDLLKIIKDKAFLDNEENEKILEDIILTLT | |
| | LFEDREMIEERLSKYADLFDKKVLKKLKRRRYTGWGRLSKKLINGIRDKQSG | |
| | KTILDFLIDDGFANRNFMQLIHDDSLTFKEEIKKAQVIGNTDSLHEHIANLA | |
| | GSPAIKKGILQTVKVVDELVKVMGRHKPENIVIEMARENQTTQKGQKNSRER | |
| | MKRIEEGIKELGSQILKEHPVENTQLQNEKLYLYYLQNGRDMYVDQELDINR | |
| | LSDYDVDHIVPQSFLKDDSIDNKVLTRSDKARGKSDNVPSEEVVKKMKNYWR | |
| | QLLNAKLITQRKFDNLTKAERGGLSELDKAGFIKRQLVETRQITKHVAQILD | |
| | SRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYS | |
| | NIMNFFKTEITLANGEIRKRPLIETNGETGEIVWDKGRDFATVRKVLSMPQV | |
| | NIVKKTEVQTGGFSKESILPKRESDKLIARKKDWDTKKYGGFDSPTVAYSVL | |
| | VVADVEKGKAKKLKTVKELVGITIMERSAFEKNPVAFLEDKGYQNIQEDKII | |
| | KLPKYSLFELENGRKRLLASAGELQKGNELALPQKYVTLLYHAKHYEKLKES | |
| | EEDEEKHLEYVTNHRDEFDEIFDQISEFSERYVLADKNLEKIEELYSKNESY | |
| | SIEELASSFINLLTFTALGAPAAFKFLGKTIDRKRYTSTKEILNSTLIHQSI | |
| | TGLYETRIDLSKLGGD | |
| CasEnd-359 | MDKPYSIGLDIGTNSVGWAVVTDEYKVPSKKFKVLGNTDRKSIKKNLWGVLL | 319 |
| | FDSGETAEATRLKRTARRRYTRRKNRILYLQEIFAEEINKVDENFFHRLEES | |
| | FLVEEDKRGDRHPIFANIVEEVAYHEQYPTIYHLRKHLADNPEKADLRLVYL | |
| | ALAHIIKFRGHFLIEGKEDVENTDIQETFKEFLEIYDNTFEDSELGEEDIDV | |
| | EEILTDKISKSRRVEKVLKLFPTEKKNSIFAEFLKLIVGLTPNFKSHENLEE | |
| | DAKLQFSKDTYEEDLEELLGQIGDEYAEIFVSAKKVYDSILLSGILTVKDSS | |
| | TKAPLSASMVERYDKHHQDLTKLKKFIRKKLPDKYKDIFFDQSKNGYAGYID | |
| | GGAKQEDFYKYLKKLLNKIEGSDYFLEKIEREDFLRKQRTFDNGSIPHQVHL | |
| | QELRAIIRNQAKYYPFLKENQDKIESILTFRIPYYVGPLARGNSRFAWLSRK | |
| | SDETITPWNFDKIIDKEKSAEAFIQRMTNFDKNLPDEKVLPKHSLLYEKFTV | |
| | YNELTKVKYIDERGEEEQFFDANLKQEIFNGVFKKYRKVTKKQLLDYLLKEF | |
| | DELRIVDISGVEDRFNASYGTYHDLKKILGGEEFLDDPKNQEMLEEIIKTLT | |
| | LFEDRKMIKKRLEKYSDILTKEQIKKLSRRRYTGWGRLSAKLLNGIRDKETN | |
| | KTILDYLIDDDNSNRNFMQLIHDDNLSFKDEIAKAQVIDDSESLHEVIANLA | |
| | GSPAIKKGILQSLKIVDEIVKVMGRYAPKNIVVEMARENQTTQKGQKNSRER | |
| | MKRLQEAMKEFGSDLLKEYPTDNTALQNDKLYLYYIQNGKDMYTGEALDIDN | |
| | LSDYDVDHIVPQSFLKDDSIDNRVLVSSKEARGKSDDVPSIDIVRKMKPFWK | |
| | KLLEAKLITQRKYDNLTKVERGSLTELDKAGFIKRQLVETRQITKHVAQILD | |
| | ERFNEEVNDDGKLIRDTKIVTLKSKLVSQFRKEFELYKVREINNYHHAHDAY | |
| | LNAVVAKALIKVYPKLESEFVYGDYPVFDVKKLFKRTDREIGKATQKKFFYS | |
| | NLMNMFKSDVKLADGKVVEKPIVDVNEETGEIAWDKQKHIATIKKVLSMPQV | |
| | NIVKKTEVQTGGFSKESILPKRDSDKLIARKKDWDTEKYGGFDSPTVAYSIL | |
| | VIADIKKGKAKKIKTTKKIIGVTIMERSAFEEDEVAFLESKGYQNIQENNLV | |
| | KIPKYTLFEIENGRKRLLASAGELQKGNELALPQHYITLLYHAKNYEKIKKE | |
| | NSHIAYSLNYVNEHREEFSKLLDQVKEFAQRYTLKDANVEKLKELFEQNEEA | |
| | DLEELAKSFINLLIFTAMGAPAAFKFIGKSIDRKRYTSTKELLNATIIHQSI | |
| | TGLYETRIDLSKLGED | |
| CasEnd-360 | MKKPYSIGLDIGTNSVGWAVVTDDYKVPAKKMKVLGNTDKSHIKKNLLGALL | 320 |
| | FDSGNTAEDRRLKRTARRRYTRRRNRILYLQEIFSEEMGKVDDSFFHRLEDS | |
| | FLVTEDKRGERHPIFGNLEEEVKYHENFPTIYHLRKYLADSPEKADLRLVYL | |
| | ALAHIIKFRGHFLIEGELDTRNNDVQRLFQEFLAVYDNTFENSSLQEQNVQV | |
| | EEILTDKISKSAKKDRVLKLFPNEKSNGRFAEFLKLIVGNQADFKKHFELEE | |
| | KAPLQFSKDTYEEELEVLLAQIGDEYAELFLSAKKLYDSILLSGILTVTDVS | |
| | TKAPLSASMIQRYNEHQMDLTQLKQFIRQKLSDKYNEVESDVSKDGYAGYID | |
| | GKTNQEAFYKYLKKLLNKIEGSGYFLDKIEREDFLRKQRTFDNGSIPHQIHL | |
| | QEMRAIIRRQAEFYPFLAENQDKIEKILTFRIPYYVGPLARGKSDFAWLSRK | |
| | SADKITPWNFDEIVDKESSAEAFINRMTNYDLYLPNQKVLPKHSLLYEKFTV | |
| | YNELTKVKYKTEQMGKTAFFDANMKQEIFDGVFKVYRKVTKDKLMDFLEKEF | |
| | DEFRIVDLTGLDKAFNASLGTYHDLRKILKDKDELDNSKNEKILEDIVLTLT | |
| | LFEDREMIRKRLENYSDLLTKEQVKKLERRHYTGWGRLSAKLIHGIRNKESR | |
| | KTILDYLIDDGNSNRNFMQLINDDALSFKEEIAKAQVIGETDNLNQVVSDIA | |
| | GSPAIKKGILQSLKIVDELVKIMGGHQPENIVVEMARENQFTNQGRRNSQQR | |
| | LKGLTDSIKEFGSQILKEHPVENSQLQNDRLFLYYLQNGRDMYTGEELDIDY | |
| | LSQYDIDHIIPQAFIKDNSIDNRVLTSSKEARGKSDDVPSKDVVRKMKSYWS | |
| | KLLSAKLITQRKFDNLTKAERGGLIDDDKAGFIKRQLVETRQITKHVARILD | |
| | ERFNTETDENNKKIRQVKIVILKSNLVSNFRKEFELYKVREINDYHHAHDAY | |
| | LNAVVGKALLGKYPQLEPEFVYGDYPHENSYKYVRKSDFEENKATAKKFFYS | |
| | NIMNFFKKDVKLADGTIVERPQVERNDENGEIIWDKDKHISNVKKVLSYPQV | |
| | NIVKKVEEQTGGFSKESILPKGNSDKLIPRKTKWDTKKYGGFDSPIVAYSVL | |
| | VIADIEKGKSKKLKTVKALVGITIMEKMTFEKDPVAFLERKGYQNIQEENII | |
| | KLPKYSLFELENGRKRLLASARELQKGNEIVLPNHLVTLLYHAKNIHKVDEK | |
| | QEDQPKHLDYVDKHKDEFKELLDVVSNFSKKYTLAEGNLEKIKELYAQNNSE | |
| | DIKELASSFINLLTFTAIGAPATFKFFDKNIDRKRYTSTTEILNATLIHQSI | |
| | TGLYETRIDLSKLGGD | |

In some embodiments, the amino acid sequence of the Cas endonuclease (or the functional fragment, functional variant, or domain thereof) comprises or consists of an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any polypeptide set forth in Table 1 or set forth in any one of SEQ ID NOS: 1-320. In some embodiments, the amino acid sequence of the Cas endonuclease (or the functional fragment, functional variant, or domain thereof) comprises or consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any polypeptide set forth in Table 1 or set forth in any one of SEQ ID NOS: 1-320. In some embodiments, the amino acid sequence of the Cas endonuclease (or the functional fragment, functional variant, or domain thereof) comprises or consists of an amino acid sequence at least about 80%, 90%, 91%, 92%, 93%, 94%, 95%, 96%, 97%, 98%, 99%, or 100% identical to the amino acid sequence of any polypeptide set forth in Table 1 or set forth in any one of SEQ ID NOS: 1-320.

In some embodiments, the amino acid sequence of the Cas endonuclease (or the functional fragment, functional variant, or domain thereof) comprises or consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of a polypeptide set forth in Table 1. In some embodiments, the amino acid sequence of the Cas endonuclease (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 1. In some embodiments, the amino acid sequence of the Cas endonuclease (or the functional fragment, functional variant, or domain thereof) comprises or consists of an amino acid sequence at least 90%, 91%, 92%, 93%, 94%, 95%, 96%, 97%, 98%, 99%, or 100% identical to the amino acid sequence of a polypeptide set forth in Table 1.

In some embodiments, the amino acid sequence of the Cas endonuclease (or the functional fragment, functional variant, or domain thereof) comprises or consists of the amino acid sequence of a polypeptide set forth in Table 1, and further comprises 1 or more but less than 20% (e.g., less than 15%, less than 12%, less than 10%, less than 8%) amino acid variations (e.g., substitutions, additions, deletions, etc.). In some embodiments, the amino acid sequence of the Cas endonuclease (or the functional fragment, functional variant, or domain thereof) comprises or consists of the amino acid sequence of a polypeptide set forth in Table 1, and further comprises or consists of at least about 1, 2, 3, 4, 5, 6, 7, 8, 9, 10, 20, 30, 40, 50, 60, 70, 80, 90, 100, 150, or 200 amino acid variations (e.g., substitutions, additions, deletions, etc.). In some embodiments, the amino acid sequence of the Cas endonuclease (or the functional fragment, functional variant, or domain thereof) comprises or consists of the amino acid sequence of a polypeptide set forth in Table 1, and further comprises or consists of about 1, 2, 3, 4, 5, 6, 7, 8, 9, 10, 20, 30, 40, 50, 60, 70, 80, 90, 100, 150, or 200 amino acid variations (e.g., substitutions, additions, deletions, etc.). In some embodiments, the amino acid sequence of the Cas endonuclease (or the functional fragment, functional variant, or domain thereof) comprises or consists of the amino acid sequence of a polypeptide set forth in Table 1, and further comprises or consists of no more than about 1, 2, 3, 4, 5, 6, 7, 8, 9, 10, 20, 30, 40, 50, 60, 70, 80, 90, 100, 150, or 200 amino acid variations (e.g., substitutions, additions, deletions, etc.). In some embodiments, the amino acid sequence of the Cas endonuclease (or the functional fragment, functional variant, or domain thereof) comprises or consists of the amino acid sequence of a polypeptide set forth in Table 1, and further comprises or consists of from about 1-200, 1-150, 1-100, 1-90, 1-80, 1-70, 1-60, 1-50, 1-320, 1-30, 1-20, 1-10, 1-5, 10-200, 10-150, 10-100, 10-90, 10-80, 10-70, 10-60, 10-50, 1-040, 10-30, 10-20, 50-200, 50-150, 50-100, 50-90, 50-80, 50-70, or 50-60 amino acid variations (e.g., substitutions, additions, deletions, etc.).

In some embodiments, the amino acid sequence of the Cas endonuclease (or the functional fragment, functional variant, or domain thereof) comprises or consists of the amino acid sequence of a polypeptide set forth in Table 1, and further comprises 1 or more but less than 20% (e.g., less than 15%, less than 12%, less than 10%, less than 8%) amino acid substitutions. In some embodiments, the amino acid sequence of the Cas endonuclease (or the functional fragment, functional variant, or domain thereof) comprises or consists of the amino acid sequence of a polypeptide set forth in Table 1, and further comprises or consists of at least about 1, 2, 3, 4, 5, 6, 7, 8, 9, 10, 20, 30, 40, 50, 60, 70, 80, 90, 100, 150, or 200 amino acid substitutions. In some embodiments, the amino acid sequence of the Cas endonuclease (or the functional fragment, functional variant, or domain thereof) comprises or consists of the amino acid sequence of a polypeptide set forth in Table 1, and further comprises or consists of about 1, 2, 3, 4, 5, 6, 7, 8, 9, 10, 20, 30, 40, 50, 60, 70, 80, 90, 100, 150, or 200 amino acid substitutions. In some embodiments, the amino acid sequence of the Cas endonuclease (or the functional fragment, functional variant, or domain thereof) comprises or consists of the amino acid sequence of a polypeptide set forth in Table 1, and further comprises or consists of no more than about 1, 2, 3, 4, 5, 6, 7, 8, 9, 10, 20, 30, 40, 50, 60, 70, 80, 90, 100, 150, or 200 amino acid substitutions. In some embodiments, the amino acid sequence of the Cas endonuclease (or the functional fragment, functional variant, or domain thereof) comprises or consists of the amino acid sequence of a polypeptide set forth in Table 1, and further comprises or consists of from about 1-200, 1-150, 1-100, 1-90, 1-80, 1-70, 1-60, 1-50, 1-320, 1-30, 1-20, 1-10, 1-5, 10-200, 10-150, 10-100, 10-90, 10-80, 10-70, 10-60, 10-50, 1-040, 10-30, 10-20, 50-200, 50-150, 50-100, 50-90, 50-80, 50-70, or 50-60 amino acid substitutions.

In some embodiments, the amino acid sequence of the Cas endonuclease (or the functional fragment, functional variant, or domain thereof) comprises or consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-320. In some embodiments, the amino acid sequence of Cas endonuclease (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-320. In some embodiments, the amino acid sequence of Cas endonuclease (or the functional fragment, functional variant, or domain thereof) comprises or consists of an amino acid sequence at least 90%, 91%, 92%, 93%, 94%, 95%, 96%, 97%, 98%, 99%, or 100% identical to the amino acid sequence of any one of SEQ ID NOS: 1-320.

In some embodiments, the amino acid sequence of the Cas endonuclease (or the functional fragment, functional variant, or domain thereof) comprises or consists of the amino acid sequence of any one of SEQ ID NOS: 1-320, and further comprises 1 or more but less than 20% (e.g., less than 15%, less than 12%, less than 10%, less than 8%) amino acid variations (e.g., substitutions, additions, deletions, etc.). In some embodiments, the amino acid sequence of the Cas endonuclease (or the functional fragment, functional variant, or domain thereof) comprises or consists of the amino acid sequence of any one of SEQ ID NOS: 1-320, and further comprises or consists of at least about 1, 2, 3, 4, 5, 6, 7, 8, 9, 10, 20, 30, 40, 50, 60, 70, 80, 90, 100, 150, or 200 amino acid variations (e.g., substitutions, additions, deletions, etc.). In some embodiments, the amino acid sequence of the Cas endonuclease (or the functional fragment, functional variant, or domain thereof) comprises or consists of the amino acid sequence of any one of SEQ ID NOS: 1-320, and further comprises or consists of about 1, 2, 3, 4, 5, 6, 7, 8, 9, 10, 20, 30, 40, 50, 60, 70, 80, 90, 100, 150, or 200 amino acid variations (e.g., substitutions, additions, deletions, etc.). In some embodiments, the amino acid sequence of the Cas endonuclease (or the functional fragment, functional variant, or domain thereof) comprises or consists of the amino acid sequence of any one of SEQ ID NOS: 1-320, and further comprises or consists of no more than about 1, 2, 3, 4, 5, 6, 7, 8, 9, 10, 20, 30, 40, 50, 60, 70, 80, 90, 100, 150, or 200 amino acid variations (e.g., substitutions, additions, deletions, etc.). In some embodiments, the amino acid sequence of the Cas endonuclease (or the functional fragment, functional variant, or domain thereof) comprises or consists of the amino acid sequence of any one of SEQ ID NOS: 1-320, and further comprises or consists of from about 1-200, 1-150, 1-100, 1-90, 1-80, 1-70, 1-60, 1-50, 1-320, 1-30, 1-20, 1-10, 1-5, 10-200, 10-150, 10-100, 10-90, 10-80, 10-70, 10-60, 10-50, 1-040, 10-30, 10-20, 50-200, 50-150, 50-100, 50-90, 50-80, 50-70, or 50-60 amino acid variations (e.g., substitutions, additions, deletions, etc.).

In some embodiments, the amino acid sequence of the Cas endonuclease (or the functional fragment, functional variant, or domain thereof) comprises or consists of the amino acid sequence of any one of SEQ ID NOS: 1-320, and further comprises 1 or more but less than 15% (less than 12%, less than 10%, less than 8%), amino acid substitutions. In some embodiments, the amino acid sequence of the Cas endonuclease (or the functional fragment, functional variant, or domain thereof) comprises or consists of the amino acid sequence of any one of SEQ ID NOS: 1-320, and further comprises or consists of at least about 1, 2, 3, 4, 5, 6, 7, 8, 9, 10, 20, 30, 40, 50, 60, 70, 80, 90, 100, 150, or 200 amino acid substitutions. In some embodiments, the amino acid sequence of the Cas endonuclease (or the functional fragment, functional variant, or domain thereof) comprises or consists of the amino acid sequence of any one of SEQ ID NOS: 1-320, and further comprises or consists of about 1, 2, 3, 4, 5, 6, 7, 8, 9, 10, 20, 30, 40, 50, 60, 70, 80, 90, 100, 150, or 200 amino acid substitutions. In some embodiments, the amino acid sequence of the Cas endonuclease (or the functional fragment, functional variant, or domain thereof) comprises or consists of the amino acid sequence of any one of SEQ ID NOS: 1-320, and further comprises or consists of no more than about 1, 2, 3, 4, 5, 6, 7, 8, 9, 10, 20, 30, 40, 50, 60, 70, 80, 90, 100, 150, or 200 amino acid substitutions. In some embodiments, the amino acid sequence of the Cas endonuclease (or the functional fragment, functional variant, or domain thereof) comprises or consists of the amino acid sequence of any one of SEQ ID NOS: 1-320, and further comprises or consists of from about 1-200, 1-150, 1-100, 1-90, 1-80, 1-70, 1-60, 1-50, 1-320, 1-30, 1-20, 1-10, 1-5, 10-200, 10-150, 10-100, 10-90, 10-80, 10-70, 10-60, 10-50, 1-040, 10-30, 10-20, 50-200, 50-150, 50-100, 50-90, 50-80, 50-70, or 50-60 amino acid substitutions.

In some embodiments, the amino acid sequence of the Cas endonuclease is less than about 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50% identical to the amino acid sequence of a reference Cas endonuclease (e.g., a reference naturally occurring Cas endonuclease). In some embodiments, the amino acid sequence of the Cas endonuclease is less than 90% (e.g., less than 89%, 88%, 87%, 86%, 85%, 84%, 83%, 82%, 81%, 80%, 79%, 78%, 77%, 76%, 75%) and greater than 50% 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% identical to the amino acid sequence of a reference Cas endonuclease (e.g., a reference naturally occurring Cas endonuclease). In some embodiments, the amino acid sequence of the Cas endonuclease is less than about 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50% identical to the amino acid sequence of a reference Cas9 endonuclease. In some embodiments, the amino acid sequence of the Cas endonuclease is less than 90% (e.g., less than 89%, 88%, 87%, 86%, 85%, 84%, 83%, 82%, 81%, 80%, 79%, 78%, 77%, 76%, 75%) and greater than 50% 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% identical to the amino acid sequence of a reference Cas9 endonuclease. In some embodiments, the amino acid sequence of the Cas endonuclease is less than about 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, or 50% identical to the amino acid sequence of a reference Cas9 endonuclease comprising the amino acid sequence set forth in SEQ ID NO: 321. In some embodiments, the amino acid sequence of the Cas endonuclease is less than 90% (e.g., less than 89%, 88%, 87%, 86%, 85%, 84%, 83%, 82%, 81%, 80%, 79%, 78%, 77%, 76%, 75%) and greater than 50% 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% identical to the amino acid sequence of a reference Cas9 endonuclease comprising the amino acid sequence set forth in SEQ ID NO: 321.

### 4.2.1 Activity of Cas Endonucleases

The Cas endonucleases described herein can have multiple functions, have domains of different function, etc. In some embodiments, the Cas endonuclease exhibits (or is engineered to exhibit) more than one (e.g., two, there, four, five, or more) different functions (e.g., described herein). In some embodiments, the Cas endonuclease does not exhibit (or is engineered to not exhibit) one or more (e.g., two, there, four, five, or more) different functions (e.g., described herein). Exemplary functions, include, but are not limited to, endonuclease activity (e.g., introduction of double and/or single strand breaks in nucleic acid sequences), RNA (e.g., gRNA) binding activity, target nucleic acid (e.g., DNA) molecule binding activity, and target nucleic acid molecule editing activity (e.g., when provided as part of a suitable system (e.g., a system described herein).

### 4.2.1.1 Endonuclease Activity

In some embodiments, the Cas endonuclease (or a functional fragment, functional variant, or domain thereof) (or a conjugate or fusion protein comprising any of the foregoing) comprises any one or more (e.g., 1, 2, 3, 4, 5, 6, or more) of the following properties (or is engineered to have one or more of the following properties): (a) DNA endonuclease activity; (b) RNA endonuclease activity; (c) DNA/RNA hybrid endonuclease activity; (d) RNA guided DNA endonuclease activity; (e) DNA guided DNA endonuclease activity; (f) RNA guided RNA endonuclease activity; (g) DNA guided RNA endonuclease activity; (h) the ability to mediate double strand breaks in a target double stranded nucleic acid (e.g., DNA) molecule; (i) the ability to mediate single strand breaks in a target double stranded nucleic acid (e.g., DNA) molecule; (j) the inability to mediate double strand breaks in a target double stranded nucleic acid (e.g., DNA) molecule; and/or (k) the ability to mediate single strand breaks in a target double stranded nucleic acid (e.g., DNA) molecule and the inability to mediate double strand breaks in a target double stranded nucleic acid (e.g., DNA) molecule (i.e., nickase activity).

In some embodiments, the Cas endonuclease (or a functional fragment, functional variant, or domain thereof) (or a conjugate or fusion protein comprising any of the foregoing) exhibits (or is engineered to exhibit) the ability to mediate double strand breaks in a target double stranded nucleic acid (e.g., DNA) molecule. In some embodiments, the Cas endonuclease (or a functional fragment, functional variant, or domain thereof) (or a conjugate or fusion protein comprising any of the foregoing) exhibits (or is engineered to exhibit) the ability to mediate single strand breaks in a target double stranded nucleic acid (e.g., DNA) molecule.

In some embodiments, the Cas endonuclease (or a functional fragment, functional variant, or domain thereof) (or a conjugate or fusion protein comprising any of the foregoing) exhibits (or is engineered to exhibit) the ability to mediate single strand breaks in a target double stranded nucleic acid (e.g., DNA) molecule and the inability to mediate double strand breaks in a target double stranded nucleic acid (e.g., DNA) molecule (i.e., nickase activity). In some embodiments, the Cas endonuclease (or a functional fragment, functional variant, or domain thereof) (or a conjugate or fusion protein comprising any of the foregoing) is capable of (or is engineered to be capable of) mediating single strand breaks at a higher frequency than double stranded breaks in a target double stranded nucleic acid (e.g., DNA) molecule. In some embodiments, the Cas endonuclease (or a functional fragment, functional variant, or domain thereof) (or a conjugate or fusion protein comprising any of the foregoing) is capable of (or is engineered to be capable of) mediating single strand breaks at a higher frequency than double stranded breaks in a target double stranded nucleic acid (e.g., DNA) molecule (e.g., at least 90%, 95%, 96%, 97%, 98%, or 99% of the breaks in a target double stranded nucleic acid (e.g., DNA) molecule are single stranded breaks; or less than 10%, 5%, 4%, 3%, 2%, or 1% of the breaks in a target double stranded nucleic acid (e.g., DNA) molecule are double stranded breaks). In some embodiments, the Cas endonuclease (or a functional fragment, functional variant, or domain thereof) (or a conjugate or fusion protein comprising any of the foregoing) mediates (or is engineered to mediate) substantially no double strand breaks in target double stranded nucleic acid (e.g., DNA) molecules. In some embodiments, the Cas endonuclease (or a functional fragment, functional variant, or domain thereof) (or a conjugate or fusion protein comprising any of the foregoing) mediates (or is engineered to mediate) no detectable double strand breaks in target double stranded nucleic acid (e.g., DNA) molecules.

4.2.1.2 gRNA Binding Activity

In some embodiments, the Cas endonuclease comprises a nucleic acid molecule binding domain. In some embodiments, the Cas endonuclease comprises a DNA binding domain. In some embodiments, the Cas endonuclease comprises an RNA binding domain. In some embodiments, the Cas endonuclease comprises a gRNA binding domain. In some embodiments, the Cas endonuclease is capable of binding a gRNA described herein. In some embodiments, the endonuclease is capable of binding a crRNA. In some embodiments, the Cas endonuclease is capable of binding a crRNA that is part of a template RNA or a sgRNA. Without wishing to be bound by theory, it is thought that the binding of the Cas endonuclease to the crRNA (e.g., a crRNA of a template RNA or a sgRNA) facilitates targeting of the Cas endonuclease to the target nucleic acid molecule (through coordination with a tracrRNA (e.g., the tracr RNA of a template RNA or a sgRNA)).

### 4.2.1.3 Target Nucleic Acid Molecule Binding Activity

In some embodiments, the Cas endonuclease comprises a domain that is capable of binding a target nucleic acid molecule (e.g., a target double stranded nucleic acid molecule (e.g., a target dsDNA molecule)). In some embodiments, the Cas endonuclease recognizes a PAM in the target nucleic acid molecule (e.g., a target double stranded nucleic acid molecule (e.g., a target dsDNA molecule)). In some embodiments, the Cas endonuclease requires a PAM to be present in or adjacent to a target site in a target nucleic acid molecule (e.g., a target double stranded nucleic acid molecule (e.g., a target dsDNA molecule)) in order to mediate cleavage of the nucleic acid molecule. In some embodiments, the PAM sequence comprises or consists of NGG.

### 4.2.1.4 Target Nucleic Acid Editing Activity

In some embodiments, when provided within a suitable system (e.g., a system described herein (see, e.g., § 4.5)), the Cas endonuclease can mediate editing (e.g., the addition, deletion, substitution, etc.) of the nucleotide sequence of a target nucleic acid molecule. In some embodiments, the Cas endonuclease exhibits increased editing efficiency relative to the editing efficiency of a reference Cas endonuclease (e.g., when provided in a suitable system (e.g., a system described herein). In some embodiments, the Cas endonuclease exhibits at least about a 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more increase in editing efficiency relative to the editing efficiency of a reference Cas endonuclease (e.g., when provided in a suitable system (e.g., a system described herein). In some embodiments, the Cas endonuclease exhibits at least about a 50%, 55%, 60%, 65%, 70%, 75%, 80%, 85%, 90%, 95%, 100%, or more increase in editing efficiency relative to the editing efficiency of a reference Cas endonuclease (e.g., when provided in a suitable system (e.g., a system described herein). In some embodiments, the Cas endonuclease exhibits an increase from about 30%-200%, 40%-200%, 50%-200%, 60%-200%, 70%-200%, 80%-200%, 90%-200%, 100%-200%, 150%-200%, 30%-150%, 40%-150%, 50%-150%, 60%-150%, 70%-150%, 80%-150%, 90%-150%, 100%-150%, 30%-100%, 40%-100%, 50%-100%, 60%-100%, 70%-100%, 80%-100%, or 90%-100%, or more increase in editing efficiency relative to the editing efficiency of a reference Cas endonuclease (e.g., when provided in a suitable system (e.g., a system described herein).

In some embodiments, the Cas endonuclease exhibits increased editing efficiency relative to the editing efficiency of a reference Cas endonuclease set forth in SEQ ID NO: 321 (e.g., when provided in a suitable system (e.g., a system described herein). In some embodiments, the Cas endonuclease exhibits at least about a 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more increase in editing efficiency relative to the editing efficiency of the reference Cas endonuclease set forth in SEQ ID NO: 321 (e.g., when provided in a suitable system (e.g., a system described herein). In some embodiments, the Cas endonuclease exhibits at least about a 50%, 55%, 60%, 65%, 70%, 75%, 80%, 85%, 90%, 95%, 100%, or more increase in editing efficiency relative to the editing efficiency of the reference Cas endonuclease set forth in SEQ ID NO: 321 (e.g., when provided in a suitable system (e.g., a system described herein). In some embodiments, the Cas endonuclease exhibits an increase from about 30%-200%, 40%-200%, 50%-200%, 60%-200%, 70%-200%, 80%-200%, 90%-200%, 100%-200%, 150%-200%, 30%-150%, 40%-150%, 50%-150%, 60%-150%, 70%-150%, 80%-150%, 90%-150%, 100%-150%, 30%-100%, 40%-100%, 50%-100%, 60%-100%, 70%-100%, 80%-100%, or 90%-100%, or more increase in editing efficiency relative to the editing efficiency of the reference Cas endonuclease set forth in SEQ ID NO: 321 (e.g., when provided in a suitable system (e.g., a system described herein).

### 4.2.1.5 Alteration of Activity

In some embodiments, the amino acid sequence of the Cas endonuclease (or a functional fragment, functional variant, or domain thereof) comprises or consists of the amino acid sequence of any Cas endonuclease set forth in Table 1 or set forth in any one of SEQ ID NOS: 1-320, and further comprises 1 or more amino acid variation (e.g., substitution, deletion, addition), wherein the one or more amino acid variation (e.g., substitution, deletion, addition) alters an activity of the Cas endonuclease (e.g., an activity described herein (e.g., induction of double strand breaks, nickase activity, gRNA binding activity, target nucleic acid binding activity, PAM recognition, etc.)). In some embodiments, the amino acid sequence of the Cas endonuclease (or a functional fragment, functional variant, or domain thereof) comprises or consists of the amino acid sequence of any Cas endonuclease set forth in Table 1 or set forth in any one of SEQ ID NOS: 1-320, and further comprises 1 or more amino acid variation (e.g., substitution, deletion, addition) but not more than 20%, not more than 15%, not more than 12%, no more than 10%, no more than 8% amino acid variation (e.g., substitution, deletion, addition), wherein the one or more amino acid variation (e.g., substitution, deletion, addition) alters an activity of the Cas endonuclease (e.g., an activity described herein (e.g., induction of double strand breaks, nickase activity, gRNA binding activity, target nucleic acid binding activity, PAM recognition, etc.)).

In some embodiments, the one or more amino acid variation (e.g., substitution, deletion, addition) reduces or eliminates the ability of the Cas endonuclease to mediate double strand breaks in a target double stranded nucleic acid (e.g., DNA) molecule. In some embodiments, a Cas endonuclease comprising the one or more amino acid variation (e.g., substitution, deletion, addition) has the ability to mediate single strand breaks in a target double stranded nucleic acid (e.g., DNA) molecule) and does not have the ability to mediate double strand breaks in a target double stranded nucleic acid (e.g., DNA) molecule. In some embodiments, the one or more amino acid variation (e.g., substitution, deletion, addition) alters the PAM nucleotide sequence recognized by the Cas endonuclease. In some embodiments, the one or more amino acid variation (e.g., substitution, deletion, addition) reduces the endonuclease activity of the Cas endonuclease by at least 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% relative to the endonuclease lacking the one or more amino acid variation (e.g., substitution, deletion, addition). In some embodiments, the one or more amino acid variation (e.g., substitution, deletion, addition) enhances the Cas endonuclease activity of the endonuclease by at least 1-fold, 2-fold, 5-fold, 10-fold, or 100-fold relative to the Cas endonuclease lacking the one or more amino acid variation (e.g., substitution, deletion, addition).

### 4.3 Cas Endonuclease Fusion Proteins & Conjugates

In some embodiments, a Cas endonuclease (or a functional fragment, functional variant, or domain thereof) described herein (or a nucleic acid molecule encoding a Cas endonuclease (or a functional fragment, functional variant, or domain thereof) described herein) is operably connected to a heterologous moiety (e.g., a heterologous protein (e.g., or a functional fragment, functional variant, or domain thereof)). As such, further provided herein are, inter alia, fusion proteins comprising a Cas endonuclease (e.g., described herein) (or a functional fragment, functional variant, or domain thereof) and one or more heterologous protein (or a functional fragment, functional variant, or domain thereof). Further provided herein are, inter alia, conjugates comprising a Cas endonuclease (e.g., described herein) (or a functional fragment, functional variant, or domain thereof) (or a nucleic acid molecule encoding a Cas endonuclease (or a functional fragment, functional variant, or domain thereof) described herein) and one or more heterologous moiety.

Heterologous moieties include, but are not limited to, proteins, peptides, small molecules, nucleic acid molecules (e.g., DNA, RNA, DNA/RNA hybrid molecules), carbohydrates, lipids, and polymers (e.g., synthetic polymers).

In some embodiments, the endonuclease (or a functional fragment, functional variant, or domain thereof) is operably connected to at least 1, 2, 3, 4, 5, 6, 7, 8, 9, or 10, or more heterologous moieties. In some embodiments, the endonuclease (or a functional fragment, functional variant, or domain thereof) is operably connected to 1, 2, 3, 4, 5, 6, 7, 8, 9, or 10, but no more than 10 heterologous moieties. In some embodiments, the endonuclease (or a functional fragment, functional variant, or domain thereof) is operably connected to no more than 1, 2, 3, 4, 5, 6, 7, 8, 9, or 10, heterologous moieties. In some embodiments, the endonuclease (or the functional fragment or functional variant thereof) is operably connected to from about 1-10, 1-9, 1-8, 1-7, 1-6, 1-5, 1-4, 1-3, or 1-2 heterologous moieties. In some embodiments, the endonuclease (or a functional fragment, functional variant, or domain thereof) is operably connected to about 1, 2, 3, 4, 5, 6, 7, 8, 9, or 10, heterologous moieties.

### 4.3.1 Heterologous Proteins

In some embodiments, the heterologous moiety is a protein. As such, as described above, provided herein are fusion proteins comprising a Cas endonuclease (e.g., described herein) (or a functional fragment, functional variant, or domain thereof) and one or more heterologous protein. It is clear from the disclosure, but for the sake of clarity, it is to be understood that the use of the term “heterologous protein” (e.g., any heterologous protein described herein) includes a full-length protein, as well as e.g., functional fragments, functional variants, and domains of the full-length protein.

In some embodiments, the fusion protein comprises more than one heterologous protein. In some embodiments, the fusion protein comprises a plurality of heterologous proteins. In some embodiments, the Cas endonuclease (or a functional fragment, functional variant, or domain thereof) is operably connected to at least 1, 2, 3, 4, 5, 6, 7, 8, 9, or 10, or more heterologous proteins. In some embodiments, the Cas endonuclease (or a functional fragment, functional variant, or domain thereof) is operably connected to at least 1, 2, 3, 4, 5, 6, 7, 8, 9, or 10, but no more than 10 heterologous proteins. In some embodiments, the Cas endonuclease (or a functional fragment, functional variant, or domain thereof) is operably connected to no more than 1, 2, 3, 4, 5, 6, 7, 8, 9, or 10, heterologous proteins. In some embodiments, the Cas endonuclease (or a functional fragment, functional variant, or domain thereof) is operably connected to from about 1-10, 1-9, 1-8, 1-7, 1-6, 1-5, 1-4, 1-3, or 1-2 heterologous proteins (or a functional fragment, functional variant, or domain thereof). In some embodiments, the Cas endonuclease (or a functional fragment, functional variant, or domain thereof) is operably connected to about 1, 2, 3, 4, 5, 6, 7, 8, 9, or 10, heterologous proteins.

Exemplary heterologous proteins include, but are not limited to, cellular localization signals (e.g., nuclear localization signal peptides, nuclear export signal peptides); detectable proteins (e.g., fluorescent proteins, protein tags (e.g., FLAG tags, HIS tags, HA tags), reporter genes); and enzymes. In some embodiments, the heterologous protein is an enzyme. In some embodiments, the heterologous protein exhibits enzymatic activity.

In some embodiments, the heterologous protein exhibits one or more of polymerase activity (e.g., reverse transcriptase activity), nucleobase editing activity (e.g., deaminase activity), enzymatic activity, epigenetic modifying activity, nucleic acid cleavage activity, nucleic acid binding activity, transcription modulation activity, methyltransferase activity, demethylase activity (e.g., histone demethylase activity), acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, ligase activity, helicase activity, or nuclease activity.

In some embodiments, the heterologous protein exhibits polymerase (e.g., reverse transcriptase) activity, nucleobase modifying activity (e.g., deaminase activity), methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, or double-strand DNA cleavage activity and nucleic acid binding activity, or any combination of the foregoing.

In some embodiments, the heterologous protein is a polymerase (e.g., a reverse transcriptase), a nucleobase editor (e.g., a deaminase), a methyltransferase, a demethylase (e.g., a histone demethylase), an acetyltransferase, a deacetylase, a kinase, a phosphatase, a ubiquitin ligase, a deubiquitinase, an adenylase, a deadenylase, a SUMOylase, a deSUMOylase, a ribosylase, a deribosylase, a myristoylase, a demyristoylase, an integrase, a transposase, a recombinase, a ligase, a helicase, or a nuclease, or a functional fragment, functional variant, or domain of the any of the foregoing.

### 4.3.1.1 Polymerases (e.g., Reverse Transcriptases (RTs))

In some embodiments, the heterologous protein exhibits polymerase (e.g., reverse transcriptase) activity. In some embodiments, the heterologous protein exhibits RNA-dependent DNA polymerase activity. In some embodiments, the heterologous protein exhibits reverse transcriptase activity.

In some embodiments, the heterologous protein is a polymerase (or a functional fragment, functional variant, or domain thereof). In some embodiments, the polymerase comprises or consists of the catalytic (e.g., polymerase (e.g., reverse transcriptase)) domain of a polymerase (e.g., a polymerase described herein (e.g., a reverse transcriptase (RT) (e.g., described herein))). In some embodiments, the polymerase comprises or consists of the catalytic (e.g., polymerase (e.g., reverse transcriptase)) domain of a polymerase (e.g., a polymerase described herein (e.g., a RT (e.g., described herein))) and the nucleic acid (e.g., RNA, DNA) binding domain of the polymerase. In some embodiments, the polymerase comprises or consists of the catalytic (e.g., polymerase (e.g., reverse transcriptase)) domain of a RT (e.g., described herein). In some embodiments, the polymerase comprises or consists of the catalytic (e.g., polymerase (e.g., reverse transcriptase)) domain of a RT (e.g., described herein) and the RNA binding domain of the RT.

In some embodiments, the polymerase comprises an RNase H domain of a RT (e.g., a RT described herein). In some embodiments, the polymerase does not contain an RNase H domain of a RT (e.g., a RT described herein). In some embodiments, the polymerase comprises a DNA dependent DNA polymerase domain of a RT (e.g., a RT described herein). In some embodiments, the polymerase does not contain a DNA dependent DNA polymerase domain of a RT (e.g., a RT described herein). In some embodiments, the DNA dependent DNA polymerase domain is the same domain as the reverse transcriptase domain (i.e., the domain has both reverse transcriptase and DNA dependent DNA polymerase activity). In some embodiments, the DNA dependent DNA polymerase domain is not the same domain as the reverse transcriptase domain.

In some embodiments, the polymerase comprises or consists of the reverse transcriptase domain of a RT (e.g., described herein), the RNA binding domain of the RT, and the RNase H domain of the RT. In some embodiments, the polymerase comprises or consists of the reverse transcriptase domain of a RT (e.g., described herein) and the RNA binding domain of the RT, and does not contain an RNase H domain of the RT. In some embodiments, the polymerase comprises or consists of the reverse transcriptase domain of a RT (e.g., described herein), the RNA binding domain of the RT, the RNase H domain of the RT, and DNA dependent DNA polymerase domain of a RT. In some embodiments, the polymerase comprises or consists of the reverse transcriptase domain of the RT (e.g., described herein), the RNA binding domain of the RT, and the RNase H domain of the RT, and does not contain a DNA dependent DNA polymerase domain of a RT.

In some embodiments, the polymerase is a RT (or a functional fragment, functional variant, or domain thereof). In some embodiments, the RT comprises or consists of the reverse transcriptase domain of a RT (e.g., described herein). In some embodiments, the RT comprises the RNA binding domain of the RT. In some embodiments, the RT comprises or consists of an RNase domain of a RT (e.g., described herein). In some embodiments, the RT does not contain an RNase domain of a RT (e.g., described herein). In some embodiments, the RT comprises a DNA dependent DNA polymerase domain of a RT (e.g., described herein). In some embodiments, the RT does not contain a DNA dependent DNA polymerase domain of a RT (e.g., described herein). In some embodiments, the DNA dependent DNA polymerase domain is the same domain as the reverse transcriptase domain (i.e., the domain has both reverse transcriptase and DNA dependent DNA polymerase activity). In some embodiments, the DNA dependent DNA polymerase domain is not the same domain as the reverse transcriptase domain.

In some embodiments, the RT comprises or consists of the reverse transcriptase domain of a RT (e.g., described herein) and the RNA binding domain of the RT. In some embodiments, the RT comprises the reverse transcriptase domain of a RT (e.g., described herein), the RNA binding domain of the RT, and the RNase domain of the RT. In some embodiments, the RT comprises the reverse transcriptase domain of a RT (e.g., described herein) and the RNA binding domain of the RT, and does not contain the RNase domain of the RT. In some embodiments, the RT comprises the reverse transcriptase domain of a RT (e.g., described herein), the RNA binding domain of the RT, the RNase domain of the RT, and the DNA dependent DNA polymerase domain of the RT. In some embodiments, the RT comprises the reverse transcriptase domain of a RT (e.g., described herein), the RNA binding domain of the RT, the RNase domain of the RT, and does not contain the DNA dependent DNA polymerase domain of the RT. In some embodiments, the RT comprises the reverse transcriptase domain of a RT (e.g., described herein) and the RNA binding domain of the RT, and does not contain the RNase domain of the RT and the DNA dependent DNA polymerase domain of the RT.

Any of the foregoing domains (e.g., reverse transcriptase domain, RNA binding domain, RNase domain, DNA dependent DNA polymerase domain) may be derived from the same or different polymerase (e.g., reverse transcriptase). Any of the foregoing domains (e.g., reverse transcriptase domain, RNA binding domain, RNase domain, DNA dependent DNA polymerase domain) may be derived from a naturally occurring reverse polymerase (e.g., reverse transcriptase) or varied (e.g., as defined herein) (e.g., comprising one or more amino acid variation) from a naturally occurring polymerase (e.g., reverse transcriptase). In some embodiments, the RT comprises a domain from more than one RT.

In some embodiments, the RT (or the functional fragment, functional variant, or domain thereof (e.g., the reverse transcriptase domain)) comprises a region that specifically recognizes a substrate RNA. For example, in some embodiments, the RT (or the functional fragment, functional variant, or domain thereof (e.g., the reverse transcriptase domain)) comprises a UTR (e.g., a 3′ UTR) that specifically recognizes a substrate RNA (e.g., a 3′ UTR from a retrotransposon (e.g., a 3′ UTR from a non-LTR retrotransposon (e.g., an RLE-type e.g., a R2 retrotransposon)). See, e.g., Luan and Eickbush, Mol Cell Biol 15, 3882-91 (1995)), the entire contents of which are incorporated herein by reference for all purposes. Exemplary 3′ UTRs from retrotransposons are described in WO2021178720 (see, e.g., Table 3), the entire contents of which are incorporated herein by reference for all purposes. In some embodiments, the RT is dimeric (e.g., homodimeric, heterodimeric). In some embodiments, the RT is monomeric.

In some embodiments, the RT comprises or consists of a full-length RT. In some embodiments, the RT comprises or consists of a functional fragment of a RT. In some embodiments, the RT comprises or consists of a functional variant of a RT. In some embodiments, the RT comprises or consists of a functional fragment and functional variant of a RT. In some embodiments, the RT comprises or consists of one or more domains of a RT. In some embodiments, the RT comprises or consists of a functional fragment of one or more domains of a RT. In some embodiments the RT comprises or consists of a functional variant of one or more domains of a RT. In some embodiments, the RT comprises or consists of a functional fragment and functional variant of one or more domains of a RT.

In some embodiments, the RT (or a functional fragment, functional variant, or domain thereof) is a naturally occurring RT. In some embodiments, the RT comprises or consists of a functional fragment of a naturally occurring RT. In some embodiments, the RT comprises or consists of a functional variant of a naturally occurring RT. In some embodiments, the RT comprises or consists of a functional fragment and functional variant of a naturally occurring RT. In some embodiments, the RT comprises or consists of one or more domains of a naturally occurring RT. In some embodiments, the RT comprises or consists of a functional fragment of one or more domains of a naturally occurring RT. In some embodiments the RT comprises or consists of a functional variant of one or more domains of a naturally occurring RT. In some embodiments, the RT comprises or consists of a functional fragment and functional variant of one or more domains of a naturally occurring RT.

In some embodiments, the RT (or a functional fragment, functional variant, or domain thereof) comprises the amino acid sequence of a naturally occurring RT. In some embodiments, the RT (or the functional fragment, functional variant, or domain thereof) comprises an amino acid sequence that comprises at least 1 amino acid variation relative to the amino acid sequence of the naturally occurring RT. In some embodiments, the amino acid sequence of the RT (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a naturally occurring RT. In some embodiments, the amino acid sequence of the RT (or the functional fragment, functional variant, or domain thereof) comprises or consists of the amino acid sequence of a naturally occurring RT, and further comprises 1 or more but less than 15% (e.g., less than 12%, less than 10%, less than 8%) amino acid variations (e.g., substitutions, additions, deletions, etc.). In some embodiments, the amino acid sequence of the RT (or the functional fragment, functional variant, or domain thereof) comprises or consists of the amino acid sequence of a naturally occurring RT, and further comprises or consists of at least about 1, 2, 3, 4, 5, 6, 7, 8, 9, or 10 amino acid variations (e.g., substitutions, additions, deletions, etc.). In some embodiments, the amino acid sequence of the RT (or the functional fragment, functional variant, or domain thereof) comprises or consists of the amino acid sequence of a naturally occurring RT, and further comprises or consists of about 1, 2, 3, 4, 5, 6, 7, 8, 9, or 10 amino acid variations (e.g., substitutions, additions, deletions, etc.). In some embodiments, the amino acid sequence of the RT (or the functional fragment, functional variant, or domain thereof) comprises or consists of the amino acid sequence of a naturally occurring RT, and further comprises or consists of no more than about 1, 2, 3, 4, 5, 6, 7, 8, 9, or 10 amino acid variations (e.g., substitutions, additions, deletions, etc.). In some embodiments, the amino acid sequence of the RT (or the functional fragment, functional variant, or domain thereof) comprises or consists of the amino acid sequence of a naturally occurring RT, and further comprises 1 or more but less than 15% (less than 12%, less than 10%, less than 8%), amino acid substitutions. In some embodiments, the amino acid sequence of the RT (or the functional fragment, functional variant, or domain thereof) comprises or consists of the amino acid sequence of a naturally occurring RT, and further comprises or consists of at least about 1, 2, 3, 4, 5, 6, 7, 8, 9, or 10 amino acid substitutions. In some embodiments, the amino acid sequence of the RT (or the functional fragment, functional variant, or domain thereof) comprises or consists of the amino acid sequence of a naturally occurring RT, and further comprises or consists of about 1, 2, 3, 4, 5, 6, 7, 8, 9, or 10 amino acid substitutions. In some embodiments, the amino acid sequence of the RT (or the functional fragment, functional variant, or domain thereof) comprises or consists of the amino acid sequence of a naturally occurring RT, and further comprises or consists of no more than about 1, 2, 3, 4, 5, 6, 7, 8, 9, or 10 amino acid substitutions.

In some embodiments, the amino acid sequence of the RT (or a functional fragment, functional variant, or domain thereof) comprises one or more amino acid variations (e.g., relative to the amino acid sequence of a naturally occurring RT) that provide one or more improved properties e.g., relative to the amino acid sequence of a naturally occurring RT), including, e.g., lower error rates, thermostability, increased processivity, increased tolerance to inhibitors, increased reverse transcriptase speed, increased tolerance of modified nucleotides, mediate addition of modified DNA nucleotides, proof reading ability, DNA dependent DNA polymerase activity, or any combination of the foregoing. See, e.g., WO2001068895 and WO2018089860, the entire contents of each of which are incorporated herein by reference for all purposes.

Naturally occurring RTs are known in the art and described herein (see, e.g., Table 2). Naturally occurring RTs include, for example, but are not limited to, viral (e.g., retroviral) reverse transcriptases, non-LTR retrotransposon reverse transcriptases (e.g., APE-type, RLE-type), LTR retrotransposon reverse transcriptases, group II intron reverse transcriptases, diversity-generating retroelement reverse transcriptases, retron reverse transcriptases, telomerases, and retroplasmids reverse transcriptases. In some embodiments, the RT (or the functional fragment, functional variant, or domain thereof) is a eukaryotic RT or a prokaryotic RT. In some embodiments, the RT (or the functional fragment, functional variant, or domain thereof) is a viral RT or a bacterial RT.

In some embodiments, the RT (or the functional fragment, functional variant, or domain thereof) is a retroviral RT. In some embodiments, the RT (or the functional fragment, functional variant, or domain thereof) is a oncoretroviris RT or a spumavirus RT. In some embodiments, the RT (or the functional fragment, functional variant, or domain thereof) is an alpharetrovirus RT, betaretrovirus RT, deltaretrovirus RT, epsilonretrovirus RT, gammaretrovirus RT, lentivirus RT, bovispumavirus RT, equispumavirus RT, felispumavirus RT, prosimiispumavirus RT, or simiispumavirus RT. In some embodiments, the RT (or the functional fragment, functional variant, or domain thereof) is a murine leukemia virus (MLV) RT, a Moloney murine leukemia virus (M-MLV) RT, a Rous sarcoma virus (RSV) RT, an avian myeloblastosis virus (AMV) RT, a human immunodeficiency virus (HIV) RT (e.g., an HIV-1 RT, an HIV-2 RT), an avian leukosis virus RT, a mouse mammary tumor virus, a feline leukemia virus, a bovine leukemia virus (ALV) RT, a human t-lymphotropic virus (HTLV) RT (e.g., an HTLV-1 RT), a simian immunodeficiency virus (SIV) RT, or a feline immunodeficiency virus (FIV) RT.

In some embodiments, the RT (or the functional fragment, functional variant, or domain thereof) is a non-LTR retrotransposon. In some embodiments, the RT (or the functional fragment, functional variant, or domain thereof) is an APE-type non-LTR retrotransposon. In some embodiments, the RT (or the functional fragment, functional variant, or domain thereof) is an APE-type non-LTR retrotransposon from the R1, or Txl clade. In some embodiments, the RT (or the functional fragment, functional variant, or domain thereof) is an RLE-type non-LTR retrotransposon. In some embodiments, the RT (or the functional fragment, functional variant, or domain thereof) is an RLE-type non-LTR retrotransposon from the R2, NeSL, HERO, R4, or CRE clade. In some embodiments, the RT (or the functional fragment, functional variant, or domain thereof) is an R2 RLE-type non-LTR retrotransposon. In some embodiments, the RT (or the functional fragment, functional variant, or domain thereof) is a RT from R2Bm non-LTR retrotransposon, a RT from R2Tg non-LTR retrotransposon, a RT from LINE-1 non-LTR retrotransposon, or RT from Penelope or a Penelope-like element (PLE) non-LTR retrotransposon.

In some embodiments, the RT (or the functional fragment, functional variant, or domain thereof) is an LTR retrotransposon (e.g., a RT from the Tyl LTR retrotransposon). In some embodiments, the RT (or the functional fragment, functional variant, or domain thereof) is a group II intron. In some embodiments, the RT (or the functional fragment or functional variant thereof) is a group II intron maturase RT from Eubacterium rectale (Marathon RT) (see, e.g., Zhao et al. RNA 24:2 2018, the entire contents of which is incorporated herein by reference for all purposes); a group II intron LtrA RT; or thermostable group II intron RT (TGIRT). In some embodiments, the RT (or the functional fragment, functional variant, or domain thereof) is a diversity-generating retroelement (e.g., from the Bordetella bacteriophage BPP-1 diversity-generating retroelement). In some embodiments, the RT (or the functional fragment, functional variant, or domain thereof) is retron reverse transcriptase (e.g., a reverse transcriptase from Ec86 (RT86)). In some embodiments, the RT (or the functional fragment, functional variant, or domain thereof) is a telomerase (e.g., a RT from a TERT telomerase). In some embodiments, the RT (or the functional fragment, functional variant, or domain thereof) is retroplasmid reverse transcriptase (e.g., the RT from a Mauriceville plasmid).

The amino acid sequence of exemplary RTs is provided in Table 2 and in SEQ ID NOS: 324-476. The accession number of each exemplary RT is also provided in Table 2.

TABLE 2
Amino Acid Sequence of Exemplary Reverse Transcriptases.

| | | SEQ |
|---|---|---|
| Description | Amino Acid Sequence | ID NO |
| MMLV p80 RT | TLNIEDEHRLHETSKEPDVSLGSTWLSDFPQAWAETGGMGLAVRQAPL | 324 |
| ADS42990.1 | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFDEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGTAGFCRLWIPGFAEMAAPLYPLTKTGTLFNW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLLI | |
| RSV RT | TVALHLAIPLKWKPDHTPVWIDQWPLPEGKLVALTQLVEKELQLGHIE | 325 |
| AAC82561.1 | PSLSCWNTPVFVIRKASGSYRLLHDLRAVNAKLVPFGAVQQGAPVLSA | |
| | LPRGWPLMVLDLKDCFFSIPLAEQDREAFAFTLPSVNNQAPARRFQWK | |
| | VLPQGMTCSPTICQLVVGQVLEPLRLKHPSLCMLHYMDDLLLAASSHD | |
| | GLEAAGEEVISTLERAGFTISPDKVQREPGVQYLGYKLGSTYVAPVGL | |
| | VAEPRIATLWDVQKLVGSLQWLRPALGIPPRLMGPFYEQLRGSDPNEA | |
| | REWNLDMKMAWREIVRLSTTAALERWDPALPLEGAVARCEQGAIGVLG | |
| | QGLSTHPRPCLWLFSTQPTKAFTAWLEVLTLLITKLRASAVRTFGKEV | |
| | DILLLPACFREDLPLPEGILLALKGFAGKIRSSDTPSIFDIARPLHVS | |
| | LKVRVTDHPVPGPTVFTDASSSTHKGVVVWREGPRWEIKEIADLGASV | |
| | QQLEARAVAMALLLWPTTPTNVVTDSAFVAKMLLKMGQEGVPSTAAAF | |
| | ILEDALSQRSAMAAVLHVRSHSEVPGFFTEGNDVADSQATFQAYPLRE | |
| | AKDLHTALHIGPRALSKACNISMQQAREVVQTCPHCNSAPALEAGVNP | |
| | RGLGPLQIWQTDFTLEPRMAPRSWLAVTVDTASSAIVVTQHGRVTSVA | |
| | VQHHWATAIAVLGRPKAIKTDNGSCFTSKSTREWLARWGIAHTTGIPG | |
| | NSQGQAMVERANRLLKDRIRVLAEGDGFMKRIPTSKQGELLAKAMYAL | |
| | NHFERGENTKTPIQKHWRPTVLTEGPPVKIRIETGEWEKGWNVLVWGR | |
| | GYAAVKNRDTDKVIWVPSRKVKPDITQKDEVTKKDEASPLFAG | |
| AMV RT | TVALHLAIPLKWKPNHTPVWIDQWPLPEGKLVALTQLVEKELQLGHIE | 326 |
| HW606680.1 | PSLSCWNTPVFVIRKASGSYRLLHDLRAVNAKLVPFGAVQQGAPVLSA | |
| | LPRGWPLMVLDLKDCFFSIPLAEQDREAFAFTLPSVNNQAPARRFQWK | |
| | VLPQGMTCSPTICQLIVGQILEPLRLKHPSLRMLHYMDDLLLAASSHD | |
| | GLEAAGEEVISTLERAGFTISPDKVQREPGVQYLGYKLGSTYVAPVGL | |
| | VAEPRIATLWDVQKLVGSLQWLRPALGIPPRLMGPFYEQLRGSDPNEA | |
| | REWNLDMKMAWREIVQLSTTAALERWDPALPLEGAVARCEQGAIGVLG | |
| | QGLSTHPRPCLWLFSTQPTKAFTAWLEVLTLLITKLRASAVRTFGKEV | |
| | DILLLPACFREDLPLPEGILLALRGFAGKIRSSDTPSIFDIARPLHVS | |
| | LKVRVTDHPVPGPTVFTDASSSTHKGVVVWREGPRWEIKEIADLGASV | |
| | QQLEARAVAMALLLWPTTPTNVVTDSAFVAKMLLKMGQEGVPSTAAAF | |
| | ILEDALSQRSAMAAVLHVRSHSEVPGFFTEGNDVADSQATFQAY | |
| HIV RT | PISPIETVPVKLKPGMDGPKVKQWPLTEEKIKALVEICTEMEKEGKIS | 327 |
| P04585 (588- | KIGPENPYNTPVFAIKKKDSTKWRKLVDFRELNKRTQDFWEVQLGIPH | |
| 1147) | PAGLKKKKSVTVLDVGDAYFSVPLDEDERKYTAFTIPSINNETPGIRY | |
| | QYNVLPQGWKGSPAIFQSSMTKILEPFRKQNPDIVIYQYMDDLYVGSD | |
| | LEIGQHRTKIEELRQHLLRWGLTTPDKKHQKEPPFLWMGYELHPDKWT | |
| | VQPIVLPEKDSWTVNDIQKLVGKLNWASQIYPGIKVRQLCKLLRGTKA | |
| | LTEVIPLTEEAELELAENREILKEPVHGVYYDPSKDLIAEIQKQGQGQ | |
| | WTYQIYQEPFKNLKTGKYARMRGAHTNDVKQLTEAVQKITTESIVIWG | |
| | KTPKFKLPIQKETWETWWTEYWQATWIPEWEFVNTPPLVKLWYQLEKE | |
| | PIVGAETFYVDGAANRETKLGKAGYVTNRGRQKVVTLTDTTNQKTELQ | |
| | AIYLALQDSGLEVNIVTDSQYALGIIQAQPDQSESELVNQIIEQLIKK | |
| | EKVYLAWVPAHKGIGGNEQVDKLVSAGIRKVL | |
| AVIRE_P03360 | TAPLEEEYRLFLEAPIQNVILLEQWKREIPKVWAEINPPGLASTQAPI | 328 |
| | HVQLLSTALPVRVRQYPITLEAKRSLRETIRKFRAAGILRPVHSPWNT | |
| | PLLPVRKSGTSEYRMVQDLREVNKRVETIHPTVPNPYTLLSLLPPDRI | |
| | WYSVLDLKDAFFCIPLAPESQLIFAFEWADAEEGESGQLTWTRLPQGF | |
| | KNSPTLFDEALNRDLQGFRLDHPSVSLLQYVDDLLIAADTQAACLSAT | |
| | RDLLMTLAELGYRVSGKKAQLCQEEVTYLGFKIHKGSRSLSNSRTQAI | |
| | LQIPVPKTKRQVREFLGTIGYCRLWIPGFAELAQPLYAATRGGNDPLV | |
| | WGEKEEEAFQSLKLALTQPPALALPSLDKPFQLFVEETSGAAKGVLTQ | |
| | ALGPWKRPVAYLSKRLDPVAAGWPRCLRAIAAAALLTREASKLTFGQD | |
| | IEITSSHNLESLLRSPPDKWLTNARITQYQVLLLDPPRVRFKQTAALN | |
| | PATLLPETDDTLPIHHCLDTLDSLTSTRPDLTDQPLAQAEATLFTDGS | |
| | SYIRDGKRYAGAAVVTLDSVIWAEPLPIGTSAQKAELIALTKALEWSK | |
| | DKSVNIYTDSRYAFATLHVHGMIYRERGLLTAGGKAIKNAPEILALLT | |
| | AVWLPKRVAVMHCKGHQKDDAPTSTGNRRADEVAREVAIRPLSTQATI | |
| | S | |
| AVIRE_P03360_ | TAPLEEEYRLFLEAPIQNVTLLEQWKREIPKVWAEINPPGLASTQAPI | 329 |
| 3mut | HVQLLSTALPVRVRQYPITLEAKRSLRETIRKFRAAGILRPVHSPWNT | |
| | PLLPVRKSGTSEYRMVQDLREVNKRVETIHPTVPNPYTLLSLLPPDRI | |
| | WYSVLDLKDAFFCIPLAPESQLIFAFEWADAEEGESGQLTWTRLPQGF | |
| | KNSPTLFNEALNRDLQGFRLDHPSVSLLQYVDDLLIAADTQAACLSAT | |
| | RDLLMTLAELGYRVSGKKAQLCQEEVTYLGFKIHKGSRSLSNSRTQAI | |
| | LQIPVPKTKRQVREFLGTIGYCRLWIPGFAELAQPLYAATRPGNDPLV | |
| | WGEKEEEAFQSLKLALTQPPALALPSLDKPFQLFVEETSGAAKGVLTQ | |
| | ALGPWKRPVAYLSKRLDPVAAGWPRCLRAIAAAALLTREASKLTFGQD | |
| | IEITSSHNLESLLRSPPDKWLTNARITQYQVLLLDPPRVRFKQTAALN | |
| | PATLLPETDDTLPIHHCLDTLDSLTSTRPDLTDQPLAQAEATLFTDGS | |
| | SYIRDGKRYAGAAVVTLDSVIWAEPLPIGTSAQKAELIALTKALEWSK | |
| | DKSVNIYTDSRYAFATLHVHGMIYRERGWLTAGGKAIKNAPEILALLT | |
| | AVWLPKRVAVMHCKGHQKDDAPTSTGNRRADEVAREVAIRPLSTQATI | |
| | S | |
| AVIRE_P03360_ | TAPLEEEYRLFLEAPIQNVTLLEQWKREIPKVWAEINPPGLASTQAPI | 330 |
| 3mutA | HVQLLSTALPVRVRQYPITLEAKRSLRETIRKFRAAGILRPVHSPWNT | |
| | PLLPVRKSGTSEYRMVQDLREVNKRVETIHPTVPNPYTLLSLLPPDRI | |
| | WYSVLDLKDAFFCIPLAPESQLIFAFEWADAEEGESGQLTWTRLPQGF | |
| | KNSPTLFNEALNRDLQGFRLDHPSVSLLQYVDDLLIAADTQAACLSAT | |
| | RDLLMTLAELGYRVSGKKAQLCQEEVTYLGFKIHKGSRSLSNSRTQAI | |
| | LQIPVPKTKRQVREFLGKIGYCRLFIPGFAELAQPLYAATRPGNDPLV | |
| | WGEKEEEAFQSLKLALTQPPALALPSLDKPFQLFVEETSGAAKGVLTQ | |
| | ALGPWKRPVAYLSKRLDPVAAGWPRCLRAIAAAALLTREASKLTFGQD | |
| | IEITSSHNLESLLRSPPDKWLTNARITQYQVLLLDPPRVRFKQTAALN | |
| | PATLLPETDDTLPIHHCLDTLDSLTSTRPDLTDQPLAQAEATLFTDGS | |
| | SYIRDGKRYAGAAVVTLDSVIWAEPLPIGTSAQKAELIALTKALEWSK | |
| | DKSVNIYTDSRYAFATLHVHGMIYRERGWLTAGGKAIKNAPEILALLT | |
| | AVWLPKRVAVMHCKGHQKDDAPTSTGNRRADEVAREVAIRPLSTQATI | |
| | S | |
| BAEVM_P10272 | TVSLQDEHRLFDIPVTTSLPDVWLQDFPQAWAETGGLGRAKCQAPIII | 331 |
| | DLKPTAVPVSIKQYPMSLEAHMGIRQHIIKFLELGVLRPCRSPWNTPL | |
| | LPVKKPGTQDYRPVQDLREINKRTVDIHPTVPNPYNLLSTLKPDYSWY | |
| | TVLDLKDAFFCLPLAPQSQELFAFEWKDPERGISGQLTWTRLPQGFKN | |
| | SPTLFDEALHRDLTDFRTQHPEVTLLQYVDDLLLAAPTKKACTQGTRH | |
| | LLQELGEKGYRASAKKAQICQTKVTYLGYILSEGKRWLTPGRIETVAR | |
| | IPPPRNPREVREFLGTAGFCRLWIPGFAELAAPLYALTKESTPFTWQT | |
| | EHQLAFEALKKALLSAPALGLPDTSKPFTLFLDERQGIAKGVLTQKLG | |
| | PWKRPVAYLSKKLDPVAAGWPPCLRIMAATAMLVKDSAKLTLGQPLTV | |
| | ITPHTLEAIVRQPPDRWITNARLTHYQALLLDTDRVQFGPPVTLNPAT | |
| | LLPVPENQPSPHDCRQVLAETHGTREDLKDQELPDADHTWYTDGSSYL | |
| | DSGTRRAGAAVVDGHNTIWAQSLPPGTSAQKAELIALTKALELSKGKK | |
| | ANIYTDSRYAFATAHTHGSIYERRGLLTSEGKEIKNKAEIIALLKALF | |
| | LPQEVAIIHCPGHQKGQDPVAVGNRQADRVARQAAMAEVLTLATEPDN | |
| | TSHIT | |
| BAEVM_P10272_ | TVSLQDEHRLFDIPVTTSLPDVWLQDFPQAWAETGGLGRAKCQAPIII | 332 |
| 3mut | DLKPTAVPVSIKQYPMSLEAHMGIRQHIIKFLELGVLRPCRSPWNTPL | |
| | LPVKKPGTQDYRPVQDLREINKRTVDIHPTVPNPYNLLSTLKPDYSWY | |
| | TVLDLKDAFFCLPLAPQSQELFAFEWKDPERGISGQLTWTRLPQGFKN | |
| | SPTLFNEALHRDLTDFRTQHPEVTLLQYVDDLLLAAPTKKACTQGTRH | |
| | LLQELGEKGYRASAKKAQICQTKVTYLGYILSEGKRWLTPGRIETVAR | |
| | IPPPRNPREVREFLGTAGFCRLWIPGFAELAAPLYALTKPSTPFTWQT | |
| | EHQLAFEALKKALLSAPALGLPDTSKPFTLFLDERQGIAKGVLTQKLG | |
| | PWKRPVAYLSKKLDPVAAGWPPCLRIMAATAMLVKDSAKLTLGQPLTV | |
| | ITPHTLEAIVRQPPDRWITNARLTHYQALLLDTDRVQFGPPVTLNPAT | |
| | LLPVPENQPSPHDCRQVLAETHGTREDLKDQELPDADHTWYTDGSSYL | |
| | DSGTRRAGAAVVDGHNTIWAQSLPPGTSAQKAELIALTKALELSKGKK | |
| | ANIYTDSRYAFATAHTHGSIYERRGWLTSEGKEIKNKAEIIALLKALF | |
| | LPQEVAIIHCPGHQKGQDPVAVGNRQADRVARQAAMAEVLTLATEPDN | |
| | TSHIT | |
| BAEVM_P10272_ | TVSLQDEHRLFDIPVTTSLPDVWLQDFPQAWAETGGLGRAKCQAPIII | 333 |
| 3mutA | DLKPTAVPVSIKQYPMSLEAHMGIRQHIIKFLELGVLRPCRSPWNTPL | |
| | LPVKKPGTQDYRPVQDLREINKRTVDIHPTVPNPYNLLSTLKPDYSWY | |
| | TVLDLKDAFFCLPLAPQSQELFAFEWKDPERGISGQLTWTRLPQGFKN | |
| | SPTLFNEALHRDLTDFRTQHPEVTLLQYVDDLLLAAPTKKACTQGTRH | |
| | LLQELGEKGYRASAKKAQICQTKVTYLGYILSEGKRWLTPGRIETVAR | |
| | IPPPRNPREVREFLGKAGFCRLFIPGFAELAAPLYALTKPSTPFTWQT | |
| | EHQLAFEALKKALLSAPALGLPDTSKPFTLFLDERQGIAKGVLTQKLG | |
| | PWKRPVAYLSKKLDPVAAGWPPCLRIMAATAMLVKDSAKLTLGQPLTV | |
| | ITPHTLEAIVRQPPDRWITNARLTHYQALLLDTDRVQFGPPVTLNPAT | |
| | LLPVPENQPSPHDCRQVLAETHGTREDLKDQELPDADHTWYTDGSSYL | |
| | DSGTRRAGAAVVDGHNTIWAQSLPPGTSAQKAELIALTKALELSKGKK | |
| | ANIYTDSRYAFATAHTHGSIYERRGWLTSEGKEIKNKAEIIALLKALF | |
| | LPQEVAIIHCPGHQKGQDPVAVGNRQADRVARQAAMAEVLTLATEPDN | |
| | TSHIT | |
| BLVAU_P25059 | GVLDAPPSHIGLEHLPPPPEVPQFPLNLERLQALQDLVHRSLEAGYIS | 334 |
| | PWDGPGNNPVFPVRKPNGAWRFVHDLRVTNALTKPIPALSPGPPDLTA | |
| | IPTHLPHIICLDLKDAFFQIPVEDRFRSYFAFTLPTPGGLQPHRRFAW | |
| | RVLPQGFINSPALFERALQEPLRQVSAAFSQSLLVSYMDDILYVSPTE | |
| | EQRLQCYQTMAAHLRDLGFQVASEKTRQTPSPVPFLGQMVHERMVTYQ | |
| | SLPTLQISSPISLHQLQTVLGDLQWVSRGTPTTRRPLQLLYSSLKGID | |
| | DPRAIIHLSPEQQQGIAELRQALSHNARSRYNEQEPLLAYVHLTRAGS | |
| | TLVLFQKGAQFPLAYFQTPLTDNQASPWGLLLLLGCQYLQAQALSSYA | |
| | KTILKYYHNLPKTSLDNWIQSSEDPRVQELLQLWPQISSQGIQPPGPW | |
| | KTLVTRAEVFLTPQFSPEPIPAALCLESDGAARRGAYCLWKDHLLDFQ | |
| | AVPAPESAQKGELAGLLAGLAAAPPEPLNIWVDSKYLYSLLRTLVLGA | |
| | WLQPDPVPSYALLYKSLLRHPAIFVGHVRSHSSASHPIASLNNYVDQL | |
| BLVAU_P25059_ | GVLDAPPSHIGLEHLPPPPEVPQFPLNLERLQALQDLVHRSLEAGYIS | 335 |
| 2mut | PWDGPGNNPVFPVRKPNGAWRFVHDLRVTNALTKPIPALSPGPPDLTA | |
| | IPTHLPHIICLDLKDAFFQIPVEDRFRSYFAFTLPTPGGLQPHRRFAW | |
| | RVLPQGFINSPALFQRALQEPLRQVSAAFSQSLLVSYMDDILYVSPTE | |
| | EQRLQCYQTMAAHLRDLGFQVASEKTRQTPSPVPFLGQMVHERMVTYQ | |
| | SLPTLQISSPISLHQLQTVLGDLQWVSRGTPTTRRPLQLLYSSLKPID | |
| | DPRAIIHLSPEQQQGIAELRQALSHNARSRYNEQEPLLAYVHLTRAGS | |
| | TLVLFQKGAQFPLAYFQTPLTDNQASPWGLLLLLGCQYLQAQALSSYA | |
| | KTILKYYHNLPKTSLDNWIQSSEDPRVQELLQLWPQISSQGIQPPGPW | |
| | KTLVTRAEVFLTPQFSPEPIPAALCLESDGAARRGAYCLWKDHLLDFQ | |
| | AVPAPESAQKGELAGLLAGLAAAPPEPLNIWVDSKYLYSLLRTLVLGA | |
| | WLQPDPVPSYALLYKSLLRHPAIFVGHVRSHSSASHPIASLNNYVDQL | |
| BLVJ_P03361 | GVLDTPPSHIGLEHLPPPPEVPQFPLNLERLQALQDLVHRSLEAGYIS | 336 |
| | PWDGPGNNPVFPVRKPNGAWRFVHDLRATNALTKPIPALSPGPPDLTA | |
| | IPTHPPHIICLDLKDAFFQIPVEDRFRFYLSFTLPSPGGLQPHRRFAW | |
| | RVLPQGFINSPALFERALQEPLRQVSAAFSQSLLVSYMDDILYASPTE | |
| | EQRSQCYQALAARLRDLGFQVASEKTSQTPSPVPFLGQMVHEQIVTYQ | |
| | SLPTLQISSPISLHQLQAVLGDLQWVSRGTPTTRRPLQLLYSSLKRHH | |
| | DPRAIIQLSPEQLQGIAELRQALSHNARSRYNEQEPLLAYVHLTRAGS | |
| | TLVLFQKGAQFPLAYFQTPLTDNQASPWGLLLLLGCQYLQTQALSSYA | |
| | KPILKYYHNLPKTSLDNWIQSSEDPRVQELLQLWPQISSQGIQPPGPW | |
| | KTLITRAEVFLTPQFSPDPIPAALCLFSDGATGRGAYCLWKDHLLDFQ | |
| | AVPAPESAQKGELAGLLAGLAAAPPEPVNIWVDSKYLYSLLRTLVLGA | |
| | WLQPDPVPSYALLYKSLLRHPAIVVGHVRSHSSASHPIASLNNYVDQL | |
| BLVJ_P03361_ | GVLDTPPSHIGLEHLPPPPEVPQFPLNLERLQALQDLVHRSLEAGYIS | 337 |
| 2mut | PWDGPGNNPVFPVRKPNGAWRFVHDLRATNALTKPIPALSPGPPDLTA | |
| | IPTHPPHIICLDLKDAFFQIPVEDRFRFYLSFTLPSPGGLQPHRRFAW | |
| | RVLPQGFINSPALFNRALQEPLRQVSAAFSQSLLVSYMDDILYASPTE | |
| | EQRSQCYQALAARLRDLGFQVASEKTSQTPSPVPFLGQMVHEQIVTYQ | |
| | SLPTLQISSPISLHQLQAVLGDLQWVSRGTPTTRRPLQLLYSSLKRHH | |
| | DPRAIIQLSPEQLQGIAELRQALSHNARSRYNEQEPLLAYVHLTRAGS | |
| | TLVLFQKGAQFPLAYFQTPLTDNQASPWGLLLLLGCQYLQTQALSSYA | |
| | KPILKYYHNLPKTSLDNWIQSSEDPRVQELLQLWPQISSQGIQPPGPW | |
| | KTLITRAEVFLTPQFSPDPIPAALCLFSDGATGRGAYCLWKDHLLDFQ | |
| | AVPAPESAQKGELAGLLAGLAAAPPEPVNIWVDSKYLYSLLRTWVLGA | |
| | WLQPDPVPSYALLYKSLLRHPAIVVGHVRSHSSASHPIASLNNYVDQL | |
| BLVJ_P03361_ | GVLDTPPSHIGLEHLPPPPEVPQFPLNLERLQALQDLVHRSLEAGYIS | 338 |
| 2mutB | PWDGPGNNPVFPVRKPNGAWRFVHDLRATNALTKPIPALSPGPPDLTA | |
| | PPTHPPHIICLDLKDAFFQIPVEDRFRFYLSFTLPSPGGLQPHRRFAW | |
| | RVLPQGFINSPALFQRALQEPLRQVSAAFSQSLLVSYMDDILYASPTE | |
| | EQRSQCYQALAARLRDLGFQVASEKTSQTPSPVPFLGQMVHEQIVTYQ | |
| | SLPTLQISSPISLHQLQAVLGDLQWVSRGTPTTRRPLQLLYSSLKRHH | |
| | DPRAIIQLSPEQLQGIAELRQALSHNARSRYNEQEPLLAYVHLTRAGS | |
| | TLVLFQKGAQFPLAYFQTPLTDNQASPWGLLLLLGCQYLQTQALSSYA | |
| | KPILKYYHNLPKTSLDNWIQSSEDPRVQELLQLWPQISSQGIQPPGPW | |
| | KTLITRAEVFLTPQFSPDPIPAALCLFSDGATGRGAYCLWKDHLLDFQ | |
| | AVPAPESAQKGELAGLLAGLAAAPPEPVNIWVDSKYLYSLLRTWVLGA | |
| | WLQPDPVPSYALLYKSLLRHPAIVVGHVRSHSSASHPIASLNNYVDQL | |
| FFV_O93209 | MDLLKPLTVERKGVKIKGYWNSQADITCVPKDLLQGEEPVRQQNVTTI | 339 |
| | HGTQEGDVYYVNLKIDGRRINTEVIGTTLDYAIITPGDVPWILKKPLE | |
| | LTIKLDLEEQQGTLLNNSILSKKGKEELKQLFEKYSALWQSWENQVGH | |
| | RRIRPHKIATGTVKPTPQKQYHINPKAKPDIQIVINDLLKQGVLIQKE | |
| | STMNTPVYPVPKPNGRWRMVLDYRAVNKVTPLIAVQNQHSYGILGSLF | |
| | KGRYKTTIDLSNGFWAHPIVPEDYWITAFTWQGKQYCWTVLPQGELNS | |
| | PGLFTGDVVDLLQGIPNVEVYVDDVYISHDSEKEHLEYLDILFNRLKE | |
| | AGYIISLKKSNIANSIVDFLGFQITNEGRGLTDTFKEKLENITAPTTL | |
| | KQLQSILGLLNFARNFIPDFTELIAPLYALIPKSTKNYVPWQIEHSTT | |
| | LETLITKLNGAEYLQGRKGDKTLIMKVNASYTTGYIRYYNEGEKKPIS | |
| | YVSIVFSKTELKFTELEKLLTTVHKGLLKALDLSMGQNIHVYSPIVSM | |
| | QNIQKTPQTAKKALASRWLSWLSYLEDPRIRFFYDPQMPALKDLPAVD | |
| | TGKDNKKHPSNFQHIFYTDGSAITSPTKEGHLNAGMGIVYFINKDGNL | |
| | QKQQEWSISLGNHTAQFAEIAAFEFALKKCLPLGGNILVVTDSNYVAK | |
| | AYNEELDVWASNGFVNNRKKPLKHISKWKSVADLKRLRPDVVVTHEPG | |
| | HQKLDSSPHAYGNNLADQLATQASFKVH | |
| FFV_O93209_ | MDLLKPLTVERKGVKIKGYWNSQADITCVPKDLLQGEEPVRQQNVTTI | 340 |
| 2mut | HGTQEGDVYYVNLKIDGRRINTEVIGTTLDYAIITPGDVPWILKKPLE | |
| | LTIKLDLEEQQGTLLNNSILSKKGKEELKQLFEKYSALWQSWENQVGH | |
| | RRIRPHKIATGTVKPTPQKQYHINPKAKPDIQIVINDLLKQGVLIQKE | |
| | STMNTPVYPVPKPNGRWRMVLDYRAVNKVTPLIAVQNQHSYGILGSLF | |
| | KGRYKTTIDLSNGFWAHPIVPEDYWITAFTWQGKQYCWTVLPQGELNS | |
| | PGLFNGDVVDLLQGIPNVEVYVDDVYISHDSEKEHLEYLDILFNRLKE | |
| | AGYIISLKKSNIANSIVDFLGFQITNEGRGLTDTFKEKLENITAPTTL | |
| | KQLQSILGLLNFARNFIPDFTELIAPLYALIPKSPKNYVPWQIEHSTT | |
| | LETLITKLNGAEYLQGRKGDKTLIMKVNASYTTGYIRYYNEGEKKPIS | |
| | YVSIVFSKTELKFTELEKLLTTVHKGLLKALDLSMGQNIHVYSPIVSM | |
| | QNIQKTPQTAKKALASRWLSWLSYLEDPRIRFFYDPQMPALKDLPAVD | |
| | TGKDNKKHPSNFQHIFYTDGSAITSPTKEGHLNAGMGIVYFINKDGNL | |
| | QKQQEWSISLGNHTAQFAEIAAFEFALKKCLPLGGNILVVTDSNYVAK | |
| | AYNEELDVWASNGFVNNRKKPLKHISKWKSVADLKRLRPDVVVTHEPG | |
| | HQKLDSSPHAYGNNLADQLATQASFKVH | |
| FFV_O93209_ | MDLLKPLTVERKGVKIKGYWNSQADITCVPKDLLQGEEPVRQQNVTTI | 341 |
| 2mutA | HGTQEGDVYYVNLKIDGRRINTEVIGTTLDYAIITPGDVPWILKKPLE | |
| | LTIKLDLEEQQGTLLNNSILSKKGKEELKQLFEKYSALWQSWENQVGH | |
| | RRIRPHKIATGTVKPTPQKQYHINPKAKPDIQIVINDLLKQGVLIQKE | |
| | STMNTPVYPVPKPNGRWRMVLDYRAVNKVTPLIAVQNQHSYGILGSLF | |
| | KGRYKTTIDLSNGFWAHPIVPEDYWITAFTWQGKQYCWTVLPQGELNS | |
| | PGLFNGDVVDLLQGIPNVEVYVDDVYISHDSEKEHLEYLDILFNRLKE | |
| | AGYIISLKKSNIANSIVDFLGFQITNEGRGLTDTFKEKLENITAPTTL | |
| | KQLQSILGKLNFARNFIPDFTELIAPLYALIPKSPKNYVPWQIEHSTT | |
| | LETLITKLNGAEYLQGRKGDKTLIMKVNASYTTGYIRYYNEGEKKPIS | |
| | YVSIVFSKTELKFTELEKLLTTVHKGLLKALDLSMGQNIHVYSPIVSM | |
| | QNIQKTPQTAKKALASRWLSWLSYLEDPRIRFFYDPQMPALKDLPAVD | |
| | TGKDNKKHPSNFQHIFYTDGSAITSPTKEGHLNAGMGIVYFINKDGNL | |
| | QKQQEWSISLGNHTAQFAEIAAFEFALKKCLPLGGNILVVTDSNYVAK | |
| | AYNEELDVWASNGFVNNRKKPLKHISKWKSVADLKRLRPDVVVTHEPG | |
| | HQKLDSSPHAYGNNLADQLATQASFKVH | |
| FFV_O93209- | VPWILKKPLELTIKLDLEEQQGTLLNNSILSKKGKEELKQLFEKYSAL | 342 |
| Pro | WQSWENQVGHRRIRPHKIATGTVKPTPQKQYHINPKAKPDIQIVINDL | |
| | LKQGVLIQKESTMNTPVYPVPKPNGRWRMVLDYRAVNKVTPLIAVQNQ | |
| | HSYGILGSLFKGRYKTTIDLSNGFWAHPIVPEDYWITAFTWQGKQYCW | |
| | TVLPQGFLNSPGLFTGDVVDLLQGIPNVEVYVDDVYISHDSEKEHLEY | |
| | LDILFNRLKEAGYIISLKKSNIANSIVDFLGFQITNEGRGLTDTFKEK | |
| | LENITAPTTLKQLQSILGLLNFARNFIPDFTELIAPLYALIPKSTKNY | |
| | VPWQIEHSTTLETLITKLNGAEYLQGRKGDKTLIMKVNASYTTGYIRY | |
| | YNEGEKKPISYVSIVFSKTELKFTELEKLLTTVHKGLLKALDLSMGQN | |
| | IHVYSPIVSMQNIQKTPQTAKKALASRWLSWLSYLEDPRIRFFYDPQM | |
| | PALKDLPAVDTGKDNKKHPSNFQHIFYTDGSAITSPTKEGHLNAGMGI | |
| | VYFINKDGNLQKQQEWSISLGNHTAQFAEIAAFEFALKKCLPLGGNIL | |
| | VVTDSNYVAKAYNEELDVWASNGFVNNRKKPLKHISKWKSVADLKRLR | |
| | PDVVVTHEPGHQKLDSSPHAYGNNLADQLATQASFKVH | |
| FFV_O93209- | VPWILKKPLELTIKLDLEEQQGTLLNNSILSKKGKEELKQLFEKYSAL | 343 |
| Pro_2mut | WQSWENQVGHRRIRPHKIATGTVKPTPQKQYHINPKAKPDIQIVINDL | |
| | LKQGVLIQKESTMNTPVYPVPKPNGRWRMVLDYRAVNKVTPLIAVQNQ | |
| | HSYGILGSLFKGRYKTTIDLSNGFWAHPIVPEDYWITAFTWQGKQYCW | |
| | TVLPQGFLNSPGLFNGDVVDLLQGIPNVEVYVDDVYISHDSEKEHLEY | |
| | LDILFNRLKEAGYIISLKKSNIANSIVDFLGFQITNEGRGLTDTFKEK | |
| | LENITAPTTLKQLQSILGLLNFARNFIPDFTELIAPLYALIPKSPKNY | |
| | VPWQIEHSTTLETLITKLNGAEYLQGRKGDKTLIMKVNASYTTGYIRY | |
| | YNEGEKKPISYVSIVFSKTELKFTELEKLLTTVHKGLLKALDLSMGQN | |
| | IHVYSPIVSMQNIQKTPQTAKKALASRWLSWLSYLEDPRIRFFYDPQM | |
| | PALKDLPAVDTGKDNKKHPSNFQHIFYTDGSAITSPTKEGHLNAGMGI | |
| | VYFINKDGNLQKQQEWSISLGNHTAQFAEIAAFEFALKKCLPLGGNIL | |
| | VVTDSNYVAKAYNEELDVWASNGFVNNRKKPLKHISKWKSVADLKRLR | |
| | PDVVVTHEPGHQKLDSSPHAYGNNLADQLATQASFKVH | |
| FFV_O93209- | VPWILKKPLELTIKLDLEEQQGTLLNNSILSKKGKEELKQLFEKYSAL | 344 |
| Pro_2mutA | WQSWENQVGHRRIRPHKIATGTVKPTPQKQYHINPKAKPDIQIVINDL | |
| | LKQGVLIQKESTMNTPVYPVPKPNGRWRMVLDYRAVNKVTPLIAVQNQ | |
| | HSYGILGSLFKGRYKTTIDLSNGFWAHPIVPEDYWITAFTWQGKQYCW | |
| | TVLPQGFLNSPGLFNGDVVDLLQGIPNVEVYVDDVYISHDSEKEHLEY | |
| | LDILFNRLKEAGYIISLKKSNIANSIVDFLGFQITNEGRGLTDTFKEK | |
| | LENITAPTTLKQLQSILGKLNFARNFIPDFTELIAPLYALIPKSPKNY | |
| | VPWQIEHSTTLETLITKLNGAEYLQGRKGDKTLIMKVNASYTTGYIRY | |
| | YNEGEKKPISYVSIVFSKTELKFTELEKLLTTVHKGLLKALDLSMGQN | |
| | IHVYSPIVSMQNIQKTPQTAKKALASRWLSWLSYLEDPRIRFFYDPQM | |
| | PALKDLPAVDTGKDNKKHPSNFQHIFYTDGSAITSPTKEGHLNAGMGI | |
| | VYFINKDGNLQKQQEWSISLGNHTAQFAEIAAFEFALKKCLPLGGNIL | |
| | VVTDSNYVAKAYNEELDVWASNGFVNNRKKPLKHISKWKSVADLKRLR | |
| | PDVVVTHEPGHQKLDSSPHAYGNNLADQLATQASFKVH | |
| FLV_P10273 | TLQLEEEYRLFEPESTQKQEMDIWLKNFPQAWAETGGMGTAHCQAPVL | 345 |
| | IQLKATATPISIRQYPMPHEAYQGIKPHIRRMLDQGILKPCQSPWNTP | |
| | LLPVKKPGTEDYRPVQDLREVNKRVEDIHPTVPNPYNLLSTLPPSHPW | |
| | YTVLDLKDAFFCLRLHSESQLLFAFEWRDPEIGLSGQLTWTRLPQGFK | |
| | NSPTLFDEALHSDLADFRVRYPALVLLQYVDDLLLAAATRTECLEGTK | |
| | ALLETLGNKGYRASAKKAQICLQEVTYLGYSLKDGQRWLTKARKEAIL | |
| | SIPVPKNSRQVREFLGTAGYCRLWIPGFAELAAPLYPLTRPGTLFQWG | |
| | TEQQLAFEDIKKALLSSPALGLPDITKPFELFIDENSGFAKGVLVQKL | |
| | GPWKRPVAYLSKKLDTVASGWPPCLRMVAAIAILVKDAGKLTLGQPLT | |
| | ILTSHPVEALVRQPPNKWLSNARMTHYQAMLLDAERVHFGPTVSLNPA | |
| | TLLPLPSGGNHHDCLQILAETHGTRPDLTDQPLPDADLTWYTDGSSFI | |
| | RNGEREAGAAVTTESEVIWAAPLPPGTSAQRAELIALTQALKMAEGKK | |
| | LTVYTDSRYAFATTHVHGEIYRRRGLLTSEGKEIKNKNEILALLEALF | |
| | LPKRLSIIHCPGHQKGDSPQAKGNRLADDTAKKAATETHSSLTVLP | |
| FLV_P10273_ | TLQLEEEYRLFEPESTQKQEMDIWLKNFPQAWAETGGMGTAHCQAPVL | 346 |
| 3mut | IQLKATATPISIRQYPMPHEAYQGIKPHIRRMLDQGILKPCQSPWNTP | |
| | LLPVKKPGTEDYRPVQDLREVNKRVEDIHPTVPNPYNLLSTLPPSHPW | |
| | YTVLDLKDAFFCLRLHSESQLLFAFEWRDPEIGLSGQLTWTRLPQGFK | |
| | NSPTLFNEALHSDLADFRVRYPALVLLQYVDDLLLAAATRTECLEGTK | |
| | ALLETLGNKGYRASAKKAQICLQEVTYLGYSLKDGQRWLTKARKEAIL | |
| | SIPVPKNSRQVREFLGTAGYCRLWIPGFAELAAPLYPLTRPGTLFQWG | |
| | TEQQLAFEDIKKALLSSPALGLPDITKPFELFIDENSGFAKGVLVQKL | |
| | GPWKRPVAYLSKKLDTVASGWPPCLRMVAAIAILVKDAGKLTLGQPLT | |
| | ILTSHPVEALVRQPPNKWLSNARMTHYQAMLLDAERVHFGPTVSLNPA | |
| | TLLPLPSGGNHHDCLQILAETHGTRPDLTDQPLPDADLTWYTDGSSFI | |
| | RNGEREAGAAVTTESEVIWAAPLPPGTSAQRAELIALTQALKMAEGKK | |
| | LTVYTDSRYAFATTHVHGEIYRRRGWLTSEGKEIKNKNEILALLEALF | |
| | LPKRLSIIHCPGHQKGDSPQAKGNRLADDTAKKAATETHSSLTVLP | |
| FLV_P10273_ | TLQLEEEYRLFEPESTQKQEMDIWLKNFPQAWAETGGMGTAHCQAPVL | 347 |
| 3mutA | IQLKATATPISIRQYPMPHEAYQGIKPHIRRMLDQGILKPCQSPWNTP | |
| | LLPVKKPGTEDYRPVQDLREVNKRVEDIHPTVPNPYNLLSTLPPSHPW | |
| | YTVLDLKDAFFCLRLHSESQLLFAFEWRDPEIGLSGQLTWTRLPQGFK | |
| | NSPTLFNEALHSDLADFRVRYPALVLLQYVDDLLLAAATRTECLEGTK | |
| | ALLETLGNKGYRASAKKAQICLQEVTYLGYSLKDGQRWLTKARKEAIL | |
| | SIPVPKNSRQVREFLGKAGYCRLFIPGFAELAAPLYPLTRPGTLFQWG | |
| | TEQQLAFEDIKKALLSSPALGLPDITKPFELFIDENSGFAKGVLVQKL | |
| | GPWKRPVAYLSKKLDTVASGWPPCLRMVAAIAILVKDAGKLTLGQPLT | |
| | ILTSHPVEALVRQPPNKWLSNARMTHYQAMLLDAERVHFGPTVSLNPA | |
| | TLLPLPSGGNHHDCLQILAETHGTRPDLTDQPLPDADLTWYTDGSSFI | |
| | RNGEREAGAAVTTESEVIWAAPLPPGTSAQRAELIALTQALKMAEGKK | |
| | LTVYTDSRYAFATTHVHGEIYRRRGWLTSEGKEIKNKNEILALLEALF | |
| | LPKRLSIIHCPGHQKGDSPQAKGNRLADDTAKKAATETHSSLTVLP | |
| FOAMV_P14350 | MNPLQLLQPLPAEIKGTKLLAHWNSGATITCIPESFLEDEQPIKKTLI | 348 |
| | KTIHGEKQQNVYYVTFKVKGRKVEAEVIASPYEYILLSPTDVPWLTQQ | |
| | PLQLTILVPLQEYQEKILSKTALPEDQKQQLKTLFVKYDNLWQHWENQ | |
| | VGHRKIRPHNIATGDYPPRPQKQYPINPKAKPSIQIVIDDLLKQGVLT | |
| | PQNSTMNTPVYPVPKPDGRWRMVLDYREVNKTIPLTAAQNQHSAGILA | |
| | TIVRQKYKTTLDLANGFWAHPITPESYWLTAFTWQGKQYCWTRLPQGF | |
| | LNSPALFTADVVDLLKEIPNVQVYVDDIYLSHDDPKEHVQQLEKVFQI | |
| | LLQAGYVVSLKKSEIGQKTVEFLGFNITKEGRGLTDTFKTKLLNITPP | |
| | KDLKQLQSILGLLNFARNFIPNFAELVQPLYNLIASAKGKYIEWSEEN | |
| | TKQLNMVIEALNTASNLEERLPEQRLVIKVNTSPSAGYVRYYNETGKK | |
| | PIMYLNYVFSKAELKFSMLEKLLTTMHKALIKAMDLAMGQEILVYSPI | |
| | VSMTKIQKTPLPERKALPIRWITWMTYLEDPRIQFHYDKTLPELKHIP | |
| | DVYTSSQSPVKHPSQYEGVFYTDGSAIKSPDPTKSNNAGMGIVHATYK | |
| | PEYQVLNQWSIPLGNHTAQMAEIAAVEFACKKALKIPGPVLVITDSFY | |
| | VAESANKELPYWKSNGFVNNKKKPLKHISKWKSIAECLSMKPDITIQH | |
| | EKGISLQIPVFILKGNALADKLATQGSYVVN | |
| FOAMV_P14350_ | MNPLQLLQPLPAEIKGTKLLAHWNSGATITCIPESFLEDEQPIKKTLI | 349 |
| 2mut | KTIHGEKQQNVYYVTFKVKGRKVEAEVIASPYEYILLSPTDVPWLTQQ | |
| | PLQLTILVPLQEYQEKILSKTALPEDQKQQLKTLFVKYDNLWQHWENQ | |
| | VGHRKIRPHNIATGDYPPRPQKQYPINPKAKPSIQIVIDDLLKQGVLT | |
| | PQNSTMNTPVYPVPKPDGRWRMVLDYREVNKTIPLTAAQNQHSAGILA | |
| | TIVRQKYKTTLDLANGFWAHPITPESYWLTAFTWQGKQYCWTRLPQGF | |
| | LNSPALFNADVVDLLKEIPNVQVYVDDIYLSHDDPKEHVQQLEKVFQI | |
| | LLQAGYVVSLKKSEIGQKTVEFLGFNITKEGRGLTDTFKTKLLNITPP | |
| | KDLKQLQSILGLLNFARNFIPNFAELVQPLYNLIAPAKGKYIEWSEEN | |
| | TKQLNMVIEALNTASNLEERLPEQRLVIKVNTSPSAGYVRYYNETGKK | |
| | PIMYLNYVFSKAELKFSMLEKLLTTMHKALIKAMDLAMGQEILVYSPI | |
| | VSMTKIQKTPLPERKALPIRWITWMTYLEDPRIQFHYDKTLPELKHIP | |
| | DVYTSSQSPVKHPSQYEGVFYTDGSAIKSPDPTKSNNAGMGIVHATYK | |
| | PEYQVLNQWSIPLGNHTAQMAEIAAVEFACKKALKIPGPVLVITDSFY | |
| | VAESANKELPYWKSNGFVNNKKKPLKHISKWKSIAECLSMKPDITIQH | |
| | EKGISLQIPVFILKGNALADKLATQGSYVVN | |
| FOAMV_P14350_ | MNPLQLLQPLPAEIKGTKLLAHWNSGATITCIPESFLEDEQPIKKTLI | 350 |
| 2mutA | KTIHGEKQQNVYYVTFKVKGRKVEAEVIASPYEYILLSPTDVPWLTQQ | |
| | PLQLTILVPLQEYQEKILSKTALPEDQKQQLKTLFVKYDNLWQHWENQ | |
| | VGHRKIRPHNIATGDYPPRPQKQYPINPKAKPSIQIVIDDLLKQGVLT | |
| | PQNSTMNTPVYPVPKPDGRWRMVLDYREVNKTIPLTAAQNQHSAGILA | |
| | TIVRQKYKTTLDLANGFWAHPITPESYWLTAFTWQGKQYCWTRLPQGF | |
| | LNSPALFNADVVDLLKEIPNVQVYVDDIYLSHDDPKEHVQQLEKVFQI | |
| | LLQAGYVVSLKKSEIGQKTVEFLGFNITKEGRGLTDTFKTKLLNITPP | |
| | KDLKQLQSILGKLNFARNFIPNFAELVQPLYNLIAPAKGKYIEWSEEN | |
| | TKQLNMVIEALNTASNLEERLPEQRLVIKVNTSPSAGYVRYYNETGKK | |
| | PIMYLNYVFSKAELKFSMLEKLLTTMHKALIKAMDLAMGQEILVYSPI | |
| | VSMTKIQKTPLPERKALPIRWITWMTYLEDPRIQFHYDKTLPELKHIP | |
| | DVYTSSQSPVKHPSQYEGVFYTDGSAIKSPDPTKSNNAGMGIVHATYK | |
| | PEYQVLNQWSIPLGNHTAQMAEIAAVEFACKKALKIPGPVLVITDSFY | |
| | VAESANKELPYWKSNGFVNNKKKPLKHISKWKSIAECLSMKPDITIQH | |
| | EKGISLQIPVFILKGNALADKLATQGSYVVN | |
| FOAMV_P14350- | VPWLTQQPLQLTILVPLQEYQEKILSKTALPEDQKQQLKTLFVKYDNL | 351 |
| Pro | WQHWENQVGHRKIRPHNIATGDYPPRPQKQYPINPKAKPSIQIVIDDL | |
| | LKQGVLTPQNSTMNTPVYPVPKPDGRWRMVLDYREVNKTIPLTAAQNQ | |
| | HSAGILATIVRQKYKTTLDLANGFWAHPITPESYWLTAFTWQGKQYCW | |
| | TRLPQGELNSPALFTADVVDLLKEIPNVQVYVDDIYLSHDDPKEHVQQ | |
| | LEKVFQILLQAGYVVSLKKSEIGQKTVEFLGFNITKEGRGLTDTFKTK | |
| | LLNITPPKDLKQLQSILGLLNFARNFIPNFAELVQPLYNLIASAKGKY | |
| | IEWSEENTKQLNMVIEALNTASNLEERLPEQRLVIKVNTSPSAGYVRY | |
| | YNETGKKPIMYLNYVFSKAELKFSMLEKLLTTMHKALIKAMDLAMGQE | |
| | ILVYSPIVSMTKIQKTPLPERKALPIRWITWMTYLEDPRIQFHYDKTL | |
| | PELKHIPDVYTSSQSPVKHPSQYEGVFYTDGSAIKSPDPTKSNNAGMG | |
| | IVHATYKPEYQVLNQWSIPLGNHTAQMAEIAAVEFACKKALKIPGPVL | |
| | VITDSFYVAESANKELPYWKSNGFVNNKKKPLKHISKWKSIAECLSMK | |
| | PDITIQHEKGISLQIPVFILKGNALADKLATQGSYVVN | |
| FOAMV_P14350- | VPWLTQQPLQLTILVPLQEYQEKILSKTALPEDQKQQLKTLFVKYDNL | 352 |
| Pro_2mut | WQHWENQVGHRKIRPHNIATGDYPPRPQKQYPINPKAKPSIQIVIDDL | |
| | LKQGVLTPQNSTMNTPVYPVPKPDGRWRMVLDYREVNKTIPLTAAQNQ | |
| | HSAGILATIVRQKYKTTLDLANGFWAHPITPESYWLTAFTWQGKQYCW | |
| | TRLPQGELNSPALFNADVVDLLKEIPNVQVYVDDIYLSHDDPKEHVQQ | |
| | LEKVFQILLQAGYVVSLKKSEIGQKTVEFLGFNITKEGRGLTDTFKTK | |
| | LLNITPPKDLKQLQSILGLLNFARNFIPNFAELVQPLYNLIAPAKGKY | |
| | IEWSEENTKQLNMVIEALNTASNLEERLPEQRLVIKVNTSPSAGYVRY | |
| | YNETGKKPIMYLNYVFSKAELKFSMLEKLLTTMHKALIKAMDLAMGQE | |
| | ILVYSPIVSMTKIQKTPLPERKALPIRWITWMTYLEDPRIQFHYDKTL | |
| | PELKHIPDVYTSSQSPVKHPSQYEGVFYTDGSAIKSPDPTKSNNAGMG | |
| | IVHATYKPEYQVLNQWSIPLGNHTAQMAEIAAVEFACKKALKIPGPVL | |
| | VITDSFYVAESANKELPYWKSNGFVNNKKKPLKHISKWKSIAECLSMK | |
| | PDITIQHEKGISLQIPVFILKGNALADKLATQGSYVVN | |
| FOAMV_P14350- | VPWLTQQPLQLTILVPLQEYQEKILSKTALPEDQKQQLKTLFVKYDNL | 353 |
| Pro_2mutA | WQHWENQVGHRKIRPHNIATGDYPPRPQKQYPINPKAKPSIQIVIDDL | |
| | LKQGVLTPQNSTMNTPVYPVPKPDGRWRMVLDYREVNKTIPLTAAQNQ | |
| | HSAGILATIVRQKYKTTLDLANGFWAHPITPESYWLTAFTWQGKQYCW | |
| | TRLPQGFLNSPALFNADVVDLLKEIPNVQVYVDDIYLSHDDPKEHVQQ | |
| | LEKVFQILLQAGYVVSLKKSEIGQKTVEFLGFNITKEGRGLTDTFKTK | |
| | LLNITPPKDLKQLQSILGKLNFARNFIPNFAELVQPLYNLIAPAKGKY | |
| | IEWSEENTKQLNMVIEALNTASNLEERLPEQRLVIKVNTSPSAGYVRY | |
| | YNETGKKPIMYLNYVFSKAELKFSMLEKLLTTMHKALIKAMDLAMGQE | |
| | ILVYSPIVSMTKIQKTPLPERKALPIRWITWMTYLEDPRIQFHYDKTL | |
| | PELKHIPDVYTSSQSPVKHPSQYEGVFYTDGSAIKSPDPTKSNNAGMG | |
| | IVHATYKPEYQVLNQWSIPLGNHTAQMAEIAAVEFACKKALKIPGPVL | |
| | VITDSFYVAESANKELPYWKSNGFVNNKKKPLKHISKWKSIAECLSMK | |
| | PDITIQHEKGISLQIPVFILKGNALADKLATQGSYVVN | |
| GALV_P21414 | VLNLEEEYRLHEKPVPSSIDPSWLQLFPTVWAERAGMGLANQVPPVVV | 354 |
| | ELRSGASPVAVRQYPMSKEAREGIRPHIQKFLDLGVLVPCRSPWNTPL | |
| | LPVKKPGTNDYRPVQDLREINKRVQDIHPTVPNPYNLLSSLPPSYTWY | |
| | SVLDLKDAFFCLRLHPNSQPLFAFEWKDPEKGNTGQLTWTRLPQGFKN | |
| | SPTLFDEALHRDLAPFRALNPQVVLLQYVDDLLVAAPTYEDCKKGTQK | |
| | LLQELSKLGYRVSAKKAQLCQREVTYLGYLLKEGKRWLTPARKATVMK | |
| | IPVPTTPRQVREFLGTAGFCRLWIPGFASLAAPLYPLTKESIPFIWTE | |
| | EHQQAFDHIKKALLSAPALALPDLTKPFTLYIDERAGVARGVLTQTLG | |
| | PWRRPVAYLSKKLDPVASGWPTCLKAVAAVALLLKDADKLTLGQNVTV | |
| | IASHSLESIVRQPPDRWMTNARMTHYQSLLLNERVSFAPPAVLNPATL | |
| | LPVESEATPVHRCSEILAEETGTRRDLEDQPLPGVPTWYTDGSSFITE | |
| | GKRRAGAPIVDGKRTVWASSLPEGTSAQKAELVALTQALRLAEGKNIN | |
| | IYTDSRYAFATAHIHGAIYKQRGLLTSAGKDIKNKEEILALLEAIHLP | |
| | RRVAIIHCPGHQRGSNPVATGNRRADEAAKQAALSTRVLAGTTKP | |
| GALV_P21414_ | VLNLEEEYRLHEKPVPSSIDPSWLQLFPTVWAERAGMGLANQVPPVVV | 355 |
| 3mut | ELRSGASPVAVRQYPMSKEAREGIRPHIQKFLDLGVLVPCRSPWNTPL | |
| | LPVKKPGTNDYRPVQDLREINKRVQDIHPTVPNPYNLLSSLPPSYTWY | |
| | SVLDLKDAFFCLRLHPNSQPLFAFEWKDPEKGNTGQLTWTRLPQGFKN | |
| | SPTLFNEALHRDLAPFRALNPQVVLLQYVDDLLVAAPTYEDCKKGTQK | |
| | LLQELSKLGYRVSAKKAQLCQREVTYLGYLLKEGKRWLTPARKATVMK | |
| | IPVPTTPRQVREFLGTAGFCRLWIPGFASLAAPLYPLTKPSIPFIWTE | |
| | EHQQAFDHIKKALLSAPALALPDLTKPFTLYIDERAGVARGVLTQTLG | |
| | PWRRPVAYLSKKLDPVASGWPTCLKAVAAVALLLKDADKLTLGQNVTV | |
| | IASHSLESIVRQPPDRWMTNARMTHYQSLLLNERVSFAPPAVLNPATL | |
| | LPVESEATPVHRCSEILAEETGTRRDLEDQPLPGVPTWYTDGSSFITE | |
| | GKRRAGAPIVDGKRTVWASSLPEGTSAQKAELVALTQALRLAEGKNIN | |
| | IYTDSRYAFATAHIHGAIYKQRGWLTSAGKDIKNKEEILALLEAIHLP | |
| | RRVAIIHCPGHQRGSNPVATGNRRADEAAKQAALSTRVLAGTTKP | |
| GALV_P21414_ | VLNLEEEYRLHEKPVPSSIDPSWLQLFPTVWAERAGMGLANQVPPVVV | 356 |
| 3mutA | ELRSGASPVAVRQYPMSKEAREGIRPHIQKFLDLGVLVPCRSPWNTPL | |
| | LPVKKPGTNDYRPVQDLREINKRVQDIHPTVPNPYNLLSSLPPSYTWY | |
| | SVLDLKDAFFCLRLHPNSQPLFAFEWKDPEKGNTGQLTWTRLPQGFKN | |
| | SPTLFNEALHRDLAPFRALNPQVVLLQYVDDLLVAAPTYEDCKKGTQK | |
| | LLQELSKLGYRVSAKKAQLCQREVTYLGYLLKEGKRWLTPARKATVMK | |
| | IPVPTTPRQVREFLGKAGFCRLFIPGFASLAAPLYPLTKPSIPFIWTE | |
| | EHQQAFDHIKKALLSAPALALPDLTKPFTLYIDERAGVARGVLTQTLG | |
| | PWRRPVAYLSKKLDPVASGWPTCLKAVAAVALLLKDADKLTLGQNVTV | |
| | IASHSLESIVRQPPDRWMTNARMTHYQSLLLNERVSFAPPAVLNPATL | |
| | LPVESEATPVHRCSEILAEETGTRRDLEDQPLPGVPTWYTDGSSFITE | |
| | GKRRAGAPIVDGKRTVWASSLPEGTSAQKAELVALTQALRLAEGKNIN | |
| | IYTDSRYAFATAHIHGAIYKQRGWLTSAGKDIKNKEEILALLEAIHLP | |
| | RRVAIIHCPGHQRGSNPVATGNRRADEAAKQAALSTRVLAGTTKP | |
| HTL1A_P03362 | AVLGLEHLPRPPQISQFPLNPERLQALQHLVRKALEAGHIEPYTGPGN | 357 |
| | NPVFPVKKANGTWRFIHDLRATNSLTIDLSSSSPGPPDLSSLPTTLAH | |
| | LQTIDLRDAFFQIPLPKQFQPYFAFTVPQQCNYGPGTRYAWKVLPQGF | |
| | KNSPTLFEMQLAHILQPIRQAFPQCTILQYMDDILLASPSHEDLLLLS | |
| | EATMASLISHGLPVSENKTQQTPGTIKFLGQIISPNHLTYDAVPTVPI | |
| | RSRWALPELQALLGEIQWVSKGTPTLRQPLHSLYCALQRHTDPRDQIY | |
| | LNPSQVQSLVQLRQALSQNCRSRLVQTLPLLGAIMLTLTGTTTVVFQS | |
| | KEQWPLVWLHAPLPHTSQCPWGQLLASAVLLLDKYTLQSYGLLCQTIH | |
| | HNISTQTFNQFIQTSDHPSVPILLHHSHRFKNLGAQTGELWNTFLKTA | |
| | APLAPVKALMPVFTLSPVIINTAPCLFSDGSTSRAAYILWDKQILSQR | |
| | SFPLPPPHKSAQRAELLGLLHGLSSARSWRCLNIFLDSKYLYHYLRTL | |
| | ALGTFQGRSSQAPFQALLPRLLSRKVVYLHHVRSHTNLPDPISRLNAL | |
| | TDALLITPVLQL | |
| HTL1A_P03362_ | AVLGLEHLPRPPQISQFPLNPERLQALQHLVRKALEAGHIEPYTGPGN | 358 |
| 2mut | NPVFPVKKANGTWRFIHDLRATNSLTIDLSSSSPGPPDLSSLPTTLAH | |
| | LQTIDLRDAFFQIPLPKQFQPYFAFTVPQQCNYGPGTRYAWKVLPQGF | |
| | KNSPTLFQMQLAHILQPIRQAFPQCTILQYMDDILLASPSHEDLLLLS | |
| | EATMASLISHGLPVSENKTQQTPGTIKFLGQIISPNHLTYDAVPTVPI | |
| | RSRWALPELQALLGEIQWVSKGTPTLRQPLHSLYCALQPHTDPRDQIY | |
| | LNPSQVQSLVQLRQALSQNCRSRLVQTLPLLGAIMLTLTGTTTVVFQS | |
| | KEQWPLVWLHAPLPHTSQCPWGQLLASAVLLLDKYTLQSYGLLCQTIH | |
| | HNISTQTFNQFIQTSDHPSVPILLHHSHRFKNLGAQTGELWNTFLKTA | |
| | APLAPVKALMPVFTLSPVIINTAPCLFSDGSTSRAAYILWDKQILSQR | |
| | SFPLPPPHKSAQRAELLGLLHGLSSARSWRCLNIFLDSKYLYHYLRTL | |
| | ALGTFQGRSSQAPFQALLPRLLSRKVVYLHHVRSHTNLPDPISRLNAL | |
| | TDALLITPVLQL | |
| HTL1A_P03362_ | AVLGLEHLPRPPQISQFPLNPERLQALQHLVRKALEAGHIEPYTGPGN | 359 |
| 2mutB | NPVFPVKKANGTWRFIHDLRATNSLTIDLSSSSPGPPDLSSPPTTLAH | |
| | LQTIDLRDAFFQIPLPKQFQPYFAFTVPQQCNYGPGTRYAWKVLPQGF | |
| | KNSPTLFQMQLAHILQPIRQAFPQCTILQYMDDILLASPSHEDLLLLS | |
| | EATMASLISHGLPVSENKTQQTPGTIKFLGQIISPNHLTYDAVPTVPI | |
| | RSRWALPELQALLGEIQWVSKGTPTLRQPLHSLYCALQPHTDPRDQIY | |
| | LNPSQVQSLVQLRQALSQNCRSRLVQTLPLLGAIMLTLTGTTTVVFQS | |
| | KEQWPLVWLHAPLPHTSQCPWGQLLASAVLLLDKYTLQSYGLLCQTIH | |
| | HNISTQTFNQFIQTSDHPSVPILLHHSHRFKNLGAQTGELWNTFLKTA | |
| | APLAPVKALMPVFTLSPVIINTAPCLFSDGSTSRAAYILWDKQILSQR | |
| | SFPLPPPHKSAQRAELLGLLHGLSSARSWRCLNIFLDSKYLYHYLRTL | |
| | ALGTFQGRSSQAPFQALLPRLLSRKVVYLHHVRSHTNLPDPISRLNAL | |
| | TDALLITPVLQL | |
| HTL1C_P14078 | AVLGLEHLPRPPEISQFPLNPERLQALQHLVRKALEAGHIEPYTGPGN | 360 |
| | NPVFPVKKANGTWRFIHDLRATNSLTIDLSSSSPGPPDLSSLPTTLAH | |
| | LQTIDLKDAFFQIPLPKQFQPYFAFTVPQQCNYGPGTRYAWRVLPQGF | |
| | KNSPTLFEMQLAHILQPIRQAFPQCTILQYMDDILLASPSHADLQLLS | |
| | EATMASLISHGLPVSENKTQQTPGTIKFLGQIISPNHLTYDAVPKVPI | |
| | RSRWALPELQALLGEIQWVSKGTPTLRQPLHSLYCALQRHTDPRDQIY | |
| | LNPSQVQSLVQLRQALSQNCRSRLVQTLPLLGAIMLTLTGTTTVVFQS | |
| | KQQWPLVWLHAPLPHTSQCPWGQLLASAVLLLDKYTLQSYGLLCQTIH | |
| | HNISTQTFNQFIQTSDHPSVPILLHHSHREKNLGAQTGELWNTFLKTT | |
| | APLAPVKALMPVFTLSPVIINTAPCLFSDGSTSQAAYILWDKHILSQR | |
| | SFPLPPPHKSAQRAELLGLLHGLSSARSWRCLNIFLDSKYLYHYLRTL | |
| | ALGTFQGRSSQAPFQALLPRLLSRKVVYLHHVRSHTNLPDPISRLNAL | |
| | TDALLITPVLQL | |
| HTL1C_P14078_ | AVLGLEHLPRPPEISQFPLNPERLQALQHLVRKALEAGHIEPYTGPGN | 361 |
| 2mut | NPVFPVKKANGTWRFIHDLRATNSLTIDLSSSSPGPPDLSSLPTTLAH | |
| | LQTIDLKDAFFQIPLPKQFQPYFAFTVPQQCNYGPGTRYAWRVLPQGF | |
| | KNSPTLFQMQLAHILQPIRQAFPQCTILQYMDDILLASPSHADLQLLS | |
| | EATMASLISHGLPVSENKTQQTPGTIKFLGQIISPNHLTYDAVPKVPI | |
| | RSRWALPELQALLGEIQWVSKGTPTLRQPLHSLYCALQPHTDPRDQIY | |
| | LNPSQVQSLVQLRQALSQNCRSRLVQTLPLLGAIMLTLTGTTTVVFQS | |
| | KQQWPLVWLHAPLPHTSQCPWGQLLASAVLLLDKYTLQSYGLLCQTIH | |
| | HNISTQTFNQFIQTSDHPSVPILLHHSHREKNLGAQTGELWNTFLKTT | |
| | APLAPVKALMPVFTLSPVIINTAPCLFSDGSTSQAAYILWDKHILSQR | |
| | SFPLPPPHKSAQRAELLGLLHGLSSARSWRCLNIFLDSKYLYHYLRTL | |
| | ALGTFQGRSSQAPFQALLPRLLSRKVVYLHHVRSHTNLPDPISRLNAL | |
| | TDALLITPVLQL | |
| HTL1L_P0C211 | GLEHLPRPPEISQFPLNPERLQALQHLVRKALEAGHIEPYTGPGNNPV | 362 |
| | FPVKKANGTWRFIHDLRATNSLTVDLSSSSPGPPDLSSLPTTLAHLQT | |
| | IDLKDAFFQIPLPKQFQPYFAFTVPQQCNYGPGTRYAWKVLPQGFKNS | |
| | PTLFEMQLASILQPIRQAFPQCVILQYMDDILLASPSPEDLQQLSEAT | |
| | MASLISHGLPVSQDKTQQTPGTIKFLGQIISPNHITYDAVPTVPIRSR | |
| | WALPELQALLGEIQWVSKGTPTLRQPLHSLYCALQGHTDPRDQIYLNP | |
| | SQVQSLMQLQQALSQNCRSRLAQTLPLLGAIMLTLTGTTTVVFQSKQQ | |
| | WPLVWLHAPLPHTSQCPWGQLLASAVLLLDKYTLQSYGLLCQTIHHNI | |
| | SIQTFNQFIQTSDHPSVPILLHHSHREKNLGAQTGELWNTFLKTAAPL | |
| | APVKALTPVFTLSPIIINTAPCLFSDGSTSQAAYILWDKHILSQRSFP | |
| | LPPPHKSAQQAELLGLLHGLSSARSWHCLNIFLDSKYLYHYLRTLALG | |
| | TFQGKSSQAPFQALLPRLLAHKVIYLHHVRSHINLPDPISKLNALTDA | |
| | LLITPIL | |
| HTL1L_P0C211_ | GLEHLPRPPEISQFPLNPERLQALQHLVRKALEAGHIEPYTGPGNNPV | 363 |
| 2mut | FPVKKANGTWRFIHDLRATNSLTVDLSSSSPGPPDLSSLPTTLAHLQT | |
| | IDLKDAFFQIPLPKQFQPYFAFTVPQQCNYGPGTRYAWKVLPQGFKNS | |
| | PTLFQMQLASILQPIRQAFPQCVILQYMDDILLASPSPEDLQQLSEAT | |
| | MASLISHGLPVSQDKTQQTPGTIKFLGQIISPNHITYDAVPTVPIRSR | |
| | WALPELQALLGEIQWVSKGTPTLRQPLHSLYCALQGHTDPRDQIYLNP | |
| | SQVQSLMQLQQALSQNCRSRLAQTLPLLGAIMLTLTGTTTVVFQSKQQ | |
| | WPLVWLHAPLPHTSQCPWGQLLASAVLLLDKYTLQSYGLLCQTIHHNI | |
| | SIQTFNQFIQTSDHPSVPILLHHSHRFKNLGAQTGELWNTFLKTAAPL | |
| | APVKALTPVFTLSPIIINTAPCLFSDGSTSQAAYILWDKHILSQRSFP | |
| | LPPPHKSAQQAELLGLLHGLSSARSWHCLNIFLDSKYLYHYLRTLAWG | |
| | TFQGKSSQAPFQALLPRLLAHKVIYLHHVRSHTNLPDPISKLNALTDA | |
| | LLITPIL | |
| HTL1L_P0C211_ | GLEHLPRPPEISQFPLNPERLQALQHLVRKALEAGHIEPYTGPGNNPV | 364 |
| 2mutB | FPVKKANGTWRFIHDLRATNSLTVDLSSSSPGPPDLSSPPTTLAHLQT | |
| | IDLKDAFFQIPLPKQFQPYFAFTVPQQCNYGPGTRYAWKVLPQGFKNS | |
| | PTLFQMQLASILQPIRQAFPQCVILQYMDDILLASPSPEDLQQLSEAT | |
| | MASLISHGLPVSQDKTQQTPGTIKFLGQIISPNHITYDAVPTVPIRSR | |
| | WALPELQALLGEIQWVSKGTPTLRQPLHSLYCALQGHTDPRDQIYLNP | |
| | SQVQSLMQLQQALSQNCRSRLAQTLPLLGAIMLTLTGTTTVVFQSKQQ | |
| | WPLVWLHAPLPHTSQCPWGQLLASAVLLLDKYTLQSYGLLCQTIHHNI | |
| | SIQTFNQFIQTSDHPSVPILLHHSHRFKNLGAQTGELWNTFLKTAAPL | |
| | APVKALTPVFTLSPIIINTAPCLFSDGSTSQAAYILWDKHILSQRSFP | |
| | LPPPHKSAQQAELLGLLHGLSSARSWHCLNIFLDSKYLYHYLRTLAWG | |
| | TFQGKSSQAPFQALLPRLLAHKVIYLHHVRSHTNLPDPISKLNALTDA | |
| | LLITPIL | |
| HTL32_Q0R5R2_ | GLEHLPPPPEVSQFPLNPERLQALTDLVSRALEAKHIEPYQGPGNNPI | 365 |
| | FPVKKPNGKWRFIHDLRATNSVTRDLASPSPGPPDLTSLPQGLPHLRT | |
| | IDLTDAFFQIPLPTIFQPYFAFTLPQPNNYGPGTRYSWRVLPQGFKNS | |
| | PTLFEQQLSHILTPVRKTFPNSLIIQYMDDILLASPAPGELAALTDKV | |
| | TNALTKEGLPLSPEKTQATPGPIHFLGQVISQDCITYETLPSINVKST | |
| | WSLAELQSMLGELQWVSKGTPVLRSSLHQLYLALRGHRDPRDTIKLTS | |
| | IQVQALRTIQKALTLNCRSRLVNQLPILALIMLRPTGTTAVLFQTKQK | |
| | WPLVWLHTPHPATSLRPWGQLLANAVIILDKYSLQHYGQVCKSFHHNI | |
| | SNQALTYYLHTSDQSSVAILLQHSHRFHNLGAQPSGPWRSLLQMPQIF | |
| | QNIDVLRPPFTISPVVINHAPCLFSDGSASKAAFIIWDRQVIHQQVLS | |
| | LPSTCSAQAGELFGLLAGLQKSQPWVALNIFLDSKFLIGHLRRMALGA | |
| | FPGPSTQCELHTQLLPLLQGKTVYVHHVRSHTLLQDPISRLNEATDAL | |
| | MLAPLLPL | |
| HTL32_Q0R5R2_ | GLEHLPPPPEVSQFPLNPERLQALTDLVSRALEAKHIEPYQGPGNNPI | 366 |
| 2mut | FPVKKPNGKWRFIHDLRATNSVTRDLASPSPGPPDLTSLPQGLPHLRT | |
| | IDLTDAFFQIPLPTIFQPYFAFTLPQPNNYGPGTRYSWRVLPQGFKNS | |
| | PTLFQQQLSHILTPVRKTFPNSLIIQYMDDILLASPAPGELAALTDKV | |
| | TNALTKEGLPLSPEKTQATPGPIHFLGQVISQDCITYETLPSINVKST | |
| | WSLAELQSMLGELQWVSKGTPVLRSSLHQLYLALRGHRDPRDTIKLTS | |
| | IQVQALRTIQKALTLNCRSRLVNQLPILALIMLRPTGTTAVLFQTKQK | |
| | WPLVWLHTPHPATSLRPWGQLLANAVIILDKYSLQHYGQVCKSFHHNI | |
| | SNQALTYYLHTSDQSSVAILLQHSHRFHNLGAQPSGPWRSLLQMPQIF | |
| | QNIDVLRPPFTISPVVINHAPCLFSDGSASKAAFIIWDRQVIHQQVLS | |
| | LPSTCSAQAGELFGLLAGLQKSQPWVALNIFLDSKFLIGHLRRMAWGA | |
| | FPGPSTQCELHTQLLPLLQGKTVYVHHVRSHTLLQDPISRLNEATDAL | |
| | MLAPLLPL | |
| HTL32_Q0R5R2_ | GLEHLPPPPEVSQFPLNPERLQALTDLVSRALEAKHIEPYQGPGNNPI | 367 |
| 2mutB | FPVKKPNGKWRFIHDLRATNSVTRDLASPSPGPPDLTSPPQGLPHLRT | |
| | IDLTDAFFQIPLPTIFQPYFAFTLPQPNNYGPGTRYSWRVLPQGFKNS | |
| | PTLFQQQLSHILTPVRKTFPNSLIIQYMDDILLASPAPGELAALTDKV | |
| | TNALTKEGLPLSPEKTQATPGPIHFLGQVISQDCITYETLPSINVKST | |
| | WSLAELQSMLGELQWVSKGTPVLRSSLHQLYLALRGHRDPRDTIKLTS | |
| | IQVQALRTIQKALTLNCRSRLVNQLPILALIMLRPTGTTAVLFQTKQK | |
| | WPLVWLHTPHPATSLRPWGQLLANAVIILDKYSLQHYGQVCKSFHHNI | |
| | SNQALTYYLHTSDQSSVAILLQHSHRFHNLGAQPSGPWRSLLQMPQIF | |
| | QNIDVLRPPFTISPVVINHAPCLFSDGSASKAAFIIWDRQVIHQQVLS | |
| | LPSTCSAQAGELFGLLAGLQKSQPWVALNIFLDSKFLIGHLRRMAWGA | |
| | FPGPSTQCELHTQLLPLLQGKTVYVHHVRSHTLLQDPISRLNEATDAL | |
| | MLAPLLPL | |
| HTL3P_Q4U0X6 | GLEHLPPPPEVSQFPLNPERLQALTDLVSRALEAKHIEPYQGPGNNPI | 368 |
| | FPVKKPNGKWRFIHDLRATNSLTRDLASPSPGPPDLTSLPQDLPHLRT | |
| | IDLTDAFFQIPLPAVFQPYFAFTLPQPNNHGPGTRYSWRVLPQGFKNS | |
| | PTLFEQQLSHILAPVRKAFPNSLIIQYMDDILLASPALRELTALTDKV | |
| | TNALTKEGLPMSLEKTQATPGSIHFLGQVISPDCITYETLPSIHVKSI | |
| | WSLAELQSMLGELQWVSKGTPVLRSSLHQLYLALRGHRDPRDTIELTS | |
| | TQVQALKTIQKALALNCRSRLVSQLPILALIILRPTGTTAVLFQTKQK | |
| | WPLVWLHTPHPATSLRPWGQLLANAIITLDKYSLQHYGQICKSFHHNI | |
| | SNQALTYYLHTSDQSSVAILLQHSHRFHNLGAQPSGPWRSLLQVPQIF | |
| | QNIDVLRPPFIISPVVIDHAPCLFSDGATSKAAFILWDKQVIHQQVLP | |
| | LPSTCSAQAGELFGLLAGLQKSKPWPALNIFLDSKFLIGHLRRMALGA | |
| | FLGPSTQCDLHARLFPLLQGKTVYVHHVRSHTLLQDPISRLNEATDAL | |
| | MLAPLLPL | |
| HTL3P_Q4U0X6_ | GLEHLPPPPEVSQFPLNPERLQALTDLVSRALEAKHIEPYQGPGNNPI | 369 |
| 2mut | FPVKKPNGKWRFIHDLRATNSLTRDLASPSPGPPDLTSLPQDLPHLRT | |
| | IDLTDAFFQIPLPAVFQPYFAFTLPQPNNHGPGTRYSWRVLPQGFKNS | |
| | PTLFQQQLSHILAPVRKAFPNSLIIQYMDDILLASPALRELTALTDKV | |
| | TNALTKEGLPMSLEKTQATPGSIHFLGQVISPDCITYETLPSIHVKSI | |
| | WSLAELQSMLGELQWVSKGTPVLRSSLHQLYLALRGHRDPRDTIELTS | |
| | TQVQALKTIQKALALNCRSRLVSQLPILALIILRPTGTTAVLFQTKQK | |
| | WPLVWLHTPHPATSLRPWGQLLANAIITLDKYSLQHYGQICKSFHHNI | |
| | SNQALTYYLHTSDQSSVAILLQHSHRFHNLGAQPSGPWRSLLQVPQIF | |
| | QNIDVLRPPFIISPVVIDHAPCLFSDGATSKAAFILWDKQVIHQQVLP | |
| | LPSTCSAQAGELFGLLAGLQKSKPWPALNIFLDSKFLIGHLRRMAWGA | |
| | FLGPSTQCDLHARLFPLLQGKTVYVHHVRSHTLLQDPISRLNEATDAL | |
| | MLAPLLPL | |
| HTL3P_Q4U0X6_ | GLEHLPPPPEVSQFPLNPERLQALTDLVSRALEAKHIEPYQGPGNNPI | 370 |
| 2mutB | FPVKKPNGKWRFIHDLRATNSLTRDLASPSPGPPDLTSPPQDLPHLRT | |
| | IDLTDAFFQIPLPAVFQPYFAFTLPQPNNHGPGTRYSWRVLPQGFKNS | |
| | PTLFQQQLSHILAPVRKAFPNSLIIQYMDDILLASPALRELTALTDKV | |
| | TNALTKEGLPMSLEKTQATPGSIHFLGQVISPDCITYETLPSIHVKSI | |
| | WSLAELQSMLGELQWVSKGTPVLRSSLHQLYLALRGHRDPRDTIELTS | |
| | TQVQALKTIQKALALNCRSRLVSQLPILALIILRPTGTTAVLFQTKQK | |
| | WPLVWLHTPHPATSLRPWGQLLANAIITLDKYSLQHYGQICKSFHHNI | |
| | SNQALTYYLHTSDQSSVAILLQHSHRFHNLGAQPSGPWRSLLQVPQIF | |
| | QNIDVLRPPFIISPVVIDHAPCLFSDGATSKAAFILWDKQVIHQQVLP | |
| | LPSTCSAQAGELFGLLAGLQKSKPWPALNIFLDSKFLIGHLRRMAWGA | |
| | FLGPSTQCDLHARLFPLLQGKTVYVHHVRSHTLLQDPISRLNEATDAL | |
| | MLAPLLPL | |
| HTLV2_P03363_ | HLPPPPQVDQFPLNLPERLQALNDLVSKALEAGHIEPYSGPGNNPVFP | 371 |
| 2mut | VKKPNGKWRFIHDLRATNAITTTLTSPSPGPPDLTSLPTALPHLQTID | |
| | LTDAFFQIPLPKQYQPYFAFTIPQPCNYGPGTRYAWTVLPQGFKNSPT | |
| | LFQQQLAAVLNPMRKMFPTSTIVQYMDDILLASPTNEELQQLSQLTLQ | |
| | ALTTHGLPISQEKTQQTPGQIRFLGQVISPNHITYESTPTIPIKSQWT | |
| | LTELQVILGEIQWVSKGTPILRKHLQSLYSALHPYRDPRACITLTPQQ | |
| | LHALHAIQQALQHNCRGRLNPALPLLGLISLSTSGTTSVIFQPKQNWP | |
| | LAWLHTPHPPTSLCPWGHLLACTILTLDKYTLQHYGQLCQSFHHNMSK | |
| | QALCDFLRNSPHPSVGILIHHMGRFHNLGSQPSGPWKTLLHLPTLLQE | |
| | PRLLRPIFTLSPVVLDTAPCLFSDGSPQKAAYVLWDQTILQQDITPLP | |
| | SHETHSAQKGELLALICGLRAAKPWPSLNIFLDSKYLIKYLHSLAIGA | |
| | FLGTSAHQTLQAALPPLLQGKTIYLHHVRSHTNLPDPISTFNEYTDSL | |
| | ILAPLVPL | |
| JSRV_P31623 | PLGTSDSPVTHADPIDWKSEEPVWVDQWPLTQEKLSAAQQLVQEQLRL | 372 |
| | GHIEPSTSAWNSPIFVIKKKSGKWRLLQDLRKVNETMMHMGALQPGLP | |
| | TPSAIPDKSYIIVIDLKDCFYTIPLAPQDCKRFAFSLPSVNFKEPMQR | |
| | YQWRVLPQGMTNSPTLCQKFVATAIAPVRQRFPQLYLVHYMDDILLAH | |
| | TDEHLLYQAFSILKQHLSLNGLVIADEKIQTHFPYNYLGFSLYPRVYN | |
| | TQLVKLQTDHLKTLNDFQKLLGDINWIRPYLKLPTYTLQPLFDILKGD | |
| | SDPASPRTLSLEGRTALQSIEEAIRQQQITYCDYQRSWGLYILPTPRA | |
| | PTGVLYQDKPLRWIYLSATPTKHLLPYYELVAKIIAKGRHEAIQYFGM | |
| | EPPFICVPYALEQQDWLFQFSDNWSIAFANYPGQITHHYPSDKLLQFA | |
| | SSHAFIFPKIVRRQPIPEATLIFTDGSSNGTAALIINHQTYYAQTSFS | |
| | SAQVVELFAVHQALLTVPTSFNLFTDSSYVVGALQMIETVPIIGTTSP | |
| | EVLNLFTLIQQVLHCRQHPCFFGHIRAHSTLPGALVQGNHTADVLTKQ | |
| | VFFQS | |
| JSRV_P31623_ | PLGTSDSPVTHADPIDWKSEEPVWVDQWPLTQEKLSAAQQLVQEQLRL | 373 |
| 2mutB | GHIEPSTSAWNSPIFVIKKKSGKWRLLQDLRKVNETMMHMGALQPGLP | |
| | TPSPIPDKSYIIVIDLKDCFYTIPLAPQDCKRFAFSLPSVNFKEPMQR | |
| | YQWRVLPQGMTNSPTLCQKFVATAIAPVRQRFPQLYLVHYMDDILLAH | |
| | TDEHLLYQAFSILKQHLSLNGLVIADEKIQTHEPYNYLGFSLYPRVYN | |
| | TQLVKLQTDHLKTLNDFQKLLGDINWIRPYLKLPTYTLQPLFDILKGD | |
| | SDPASPRTLSLEGRTALQSIEEAIRQQQITYCDYQRSWGLYILPTPRA | |
| | PTGVLYQDKPLRWIYLSATPTKHLLPYYELVAKIIAKGRHEAIQYFGM | |
| | EPPFICVPYALEQQDWLFQFSDNWSIAFANYPGQITHHYPSDKLLQFA | |
| | SSHAFIFPKIVRRQPIPEATLIFTDGSSNGTAALIINHQTYYAQTSFS | |
| | SAQVVELFAVHQALLTVPTSFNLFTDSSYVVGALQMIETVPIIGTTSP | |
| | EVLNLFTLIQQVLHCRQHPCFFGHIRAHSTLPGALVQGNHTADVLTKQ | |
| | VFFQS | |
| KORV_Q9TTC1 | TLGDQGSRGSDPLPEPRVTLTVEGIPTEFLVNTGAEHSVLTKPMGKMG | 374 |
| | SKRTVVAGATGSKVYPWTTKRLLKIGQKQVTHSFLVIPECPAPLLGRD | |
| | LLTKLKAQIQFSTEGPQVTWEDRPAMCLVLNLEEEYRLHEKPVPPSID | |
| | PSWLQLFPMVWAEKAGMGLANQVPPVVVELKSDASPVAVRQYPMSKEA | |
| | REGIRPHIQRFLDLGILVPCQSPWNTPLLPVKKPGTNDYRPVQDLREV | |
| | NKRVQDIHPTVPNPYNLLSSLPPSHTWYSVLDLKDAFFCLKLHPNSQP | |
| | LFAFEWRDPEKGNTGQLTWTRLPQGFKNSPTLFDEALHRDLASFRALN | |
| | PQVVMLQYVDDLLVAAPTYRDCKEGTRRLLQELSKLGYRVSAKKAQLC | |
| | REEVTYLGYLLKGGKRWLTPARKATVMKIPTPTTPRQVREFLGTAGFC | |
| | RLWIPGFASLAAPLYPLTREKVPFTWTEAHQEAFGRIKEALLSAPALA | |
| | LPDLTKPFALYVDEKEGVARGVLTQTLGPWRRPVAYLSKKLDPVASGW | |
| | PTCLKAIAAVALLLKDADKLTLGQNVLVIAPHNLESIVRQPPDRWMTN | |
| | ARMTHYQSLLLNERVSFAPPAILNPATLLPVESDDTPIHICSEILAEE | |
| | TGTRPDLRDQPLPGVPAWYTDGSSFIMDGRRQAGAAIVDNKRTVWASN | |
| | LPEGTSAQKAELIALTQALRLAEGKSINIYTDSRYAFATAHVHGAIYK | |
| | QRGLLTSAGKDIKNKEEILALLEAIHLPKRVAIIHCPGHQRGTDPVAT | |
| | GNRKADEAAKQAAQSTRILTETTKN | |
| KORV_Q9TTC1_ | TLGDQGSRGSDPLPEPRVTLTVEGIPTEFLVNTGAEHSVLTKPMGKMG | 375 |
| 3mut | SKRTVVAGATGSKVYPWTTKRLLKIGQKQVTHSFLVIPECPAPLLGRD | |
| | LLTKLKAQIQFSTEGPQVTWEDRPAMCLVLNLEEEYRLHEKPVPPSID | |
| | PSWLQLFPMVWAEKAGMGLANQVPPVVVELKSDASPVAVRQYPMSKEA | |
| | REGIRPHIQRFLDLGILVPCQSPWNTPLLPVKKPGTNDYRPVQDLREV | |
| | NKRVQDIHPTVPNPYNLLSSLPPSHTWYSVLDLKDAFFCLKLHPNSQP | |
| | LFAFEWRDPEKGNTGQLTWTRLPQGFKNSPTLFNEALHRDLASFRALN | |
| | PQVVMLQYVDDLLVAAPTYRDCKEGTRRLLQELSKLGYRVSAKKAQLC | |
| | REEVTYLGYLLKGGKRWLTPARKATVMKIPTPTTPRQVREFLGTAGFC | |
| | RLWIPGFASLAAPLYPLTRPKVPFTWTEAHQEAFGRIKEALLSAPALA | |
| | LPDLTKPFALYVDEKEGVARGVLTQTLGPWRRPVAYLSKKLDPVASGW | |
| | PTCLKAIAAVALLLKDADKLTLGQNVLVIAPHNLESIVRQPPDRWMIN | |
| | ARMTHYQSLLLNERVSFAPPAILNPATLLPVESDDTPIHICSEILAEE | |
| | TGTRPDLRDQPLPGVPAWYTDGSSFIMDGRRQAGAAIVDNKRTVWASN | |
| | LPEGTSAQKAELIALTQALRLAEGKSINIYTDSRYAFATAHVHGAIYK | |
| | QRGWLTSAGKDIKNKEEILALLEAIHLPKRVAIIHCPGHQRGTDPVAT | |
| | GNRKADEAAKQAAQSTRILTETTKN | |
| KORV_Q9TTC1_ | TLGDQGSRGSDPLPEPRVTLTVEGIPTEFLVNTGAEHSVLTKPMGKMG | 376 |
| 3mutA | SKRTVVAGATGSKVYPWTTKRLLKIGQKQVTHSFLVIPECPAPLLGRD | |
| | LLTKLKAQIQFSTEGPQVTWEDRPAMCLVLNLEEEYRLHEKPVPPSID | |
| | PSWLQLFPMVWAEKAGMGLANQVPPVVVELKSDASPVAVRQYPMSKEA | |
| | REGIRPHIQRFLDLGILVPCQSPWNTPLLPVKKPGTNDYRPVQDLREV | |
| | NKRVQDIHPTVPNPYNLLSSLPPSHTWYSVLDLKDAFFCLKLHPNSQP | |
| | LFAFEWRDPEKGNTGQLTWTRLPQGFKNSPTLFNEALHRDLASFRALN | |
| | PQVVMLQYVDDLLVAAPTYRDCKEGTRRLLQELSKLGYRVSAKKAQLC | |
| | REEVTYLGYLLKGGKRWLTPARKATVMKIPTPTTPRQVREFLGKAGFC | |
| | RLFIPGFASLAAPLYPLTRPKVPFTWTEAHQEAFGRIKEALLSAPALA | |
| | LPDLTKPFALYVDEKEGVARGVLTQTLGPWRRPVAYLSKKLDPVASGW | |
| | PTCLKAIAAVALLLKDADKLTLGQNVLVIAPHNLESIVRQPPDRWMTN | |
| | ARMTHYQSLLLNERVSFAPPAILNPATLLPVESDDTPIHICSEILAEE | |
| | TGTRPDLRDQPLPGVPAWYTDGSSFIMDGRRQAGAAIVDNKRTVWASN | |
| | LPEGTSAQKAELIALTQALRLAEGKSINIYTDSRYAFATAHVHGAIYK | |
| | QRGWLTSAGKDIKNKEEILALLEAIHLPKRVAIIHCPGHQRGTDPVAT | |
| | GNRKADEAAKQAAQSTRILTETTKN | |
| KORV_Q9TTC1- | LLGRDLLTKLKAQIQFSTEGPQVTWEDRPAMCLVLNLEEEYRLHEKPV | 377 |
| Pro | PPSIDPSWLQLFPMVWAEKAGMGLANQVPPVVVELKSDASPVAVRQYP | |
| | MSKEAREGIRPHIQRFLDLGILVPCQSPWNTPLLPVKKPGTNDYRPVQ | |
| | DLREVNKRVQDIHPTVPNPYNLLSSLPPSHTWYSVLDLKDAFFCLKLH | |
| | PNSQPLFAFEWRDPEKGNTGQLTWTRLPQGFKNSPTLFDEALHRDLAS | |
| | FRALNPQVVMLQYVDDLLVAAPTYRDCKEGTRRLLQELSKLGYRVSAK | |
| | KAQLCREEVTYLGYLLKGGKRWLTPARKATVMKIPTPTTPRQVREFLG | |
| | TAGFCRLWIPGFASLAAPLYPLTREKVPFTWTEAHQEAFGRIKEALLS | |
| | APALALPDLTKPFALYVDEKEGVARGVLTQTLGPWRRPVAYLSKKLDP | |
| | VASGWPTCLKAIAAVALLLKDADKLTLGQNVLVIAPHNLESIVRQPPD | |
| | RWMTNARMTHYQSLLLNERVSFAPPAILNPATLLPVESDDTPIHICSE | |
| | ILAEETGTRPDLRDQPLPGVPAWYTDGSSFIMDGRRQAGAAIVDNKRT | |
| | VWASNLPEGTSAQKAELIALTQALRLAEGKSINIYTDSRYAFATAHVH | |
| | GAIYKQRGLLTSAGKDIKNKEEILALLEAIHLPKRVAIIHCPGHQRGT | |
| | DPVATGNRKADEAAKQAAQSTRILTETTKN | |
| KORV_Q9TTC1- | LLGRDLLTKLKAQIQFSTEGPQVTWEDRPAMCLVLNLEEEYRLHEKPV | 378 |
| Pro_3mut | PPSIDPSWLQLFPMVWAEKAGMGLANQVPPVVVELKSDASPVAVRQYP | |
| | MSKEAREGIRPHIQRFLDLGILVPCQSPWNTPLLPVKKPGTNDYRPVQ | |
| | DLREVNKRVQDIHPTVPNPYNLLSSLPPSHTWYSVLDLKDAFFCLKLH | |
| | PNSQPLFAFEWRDPEKGNTGQLTWTRLPQGFKNSPTLFNEALHRDLAS | |
| | FRALNPQVVMLQYVDDLLVAAPTYRDCKEGTRRLLQELSKLGYRVSAK | |
| | KAQLCREEVTYLGYLLKGGKRWLTPARKATVMKIPTPTTPRQVREFLG | |
| | TAGFCRLWIPGFASLAAPLYPLTRPKVPFTWTEAHQEAFGRIKEALLS | |
| | APALALPDLTKPFALYVDEKEGVARGVLTQTLGPWRRPVAYLSKKLDP | |
| | VASGWPTCLKAIAAVALLLKDADKLTLGQNVLVIAPHNLESIVRQPPD | |
| | RWMTNARMTHYQSLLLNERVSFAPPAILNPATLLPVESDDTPIHICSE | |
| | ILAEETGTRPDLRDQPLPGVPAWYTDGSSFIMDGRRQAGAAIVDNKRT | |
| | VWASNLPEGTSAQKAELIALTQALRLAEGKSINIYTDSRYAFATAHVH | |
| | GAIYKQRGWLTSAGKDIKNKEEILALLEAIHLPKRVAIIHCPGHQRGT | |
| | DPVATGNRKADEAAKQAAQSTRILTETTKN | |
| KORV_Q9TTC1- | LLGRDLLTKLKAQIQFSTEGPQVTWEDRPAMCLVLNLEEEYRLHEKPV | 379 |
| Pro_3mutA | PPSIDPSWLQLFPMVWAEKAGMGLANQVPPVVVELKSDASPVAVRQYP | |
| | MSKEAREGIRPHIQRFLDLGILVPCQSPWNTPLLPVKKPGTNDYRPVQ | |
| | DLREVNKRVQDIHPTVPNPYNLLSSLPPSHTWYSVLDLKDAFFCLKLH | |
| | PNSQPLFAFEWRDPEKGNTGQLTWTRLPQGFKNSPTLFNEALHRDLAS | |
| | FRALNPQVVMLQYVDDLLVAAPTYRDCKEGTRRLLQELSKLGYRVSAK | |
| | KAQLCREEVTYLGYLLKGGKRWLTPARKATVMKIPTPTTPRQVREFLG | |
| | KAGFCRLFIPGFASLAAPLYPLTRPKVPFTWTEAHQEAFGRIKEALLS | |
| | APALALPDLTKPFALYVDEKEGVARGVLTQTLGPWRRPVAYLSKKLDP | |
| | VASGWPTCLKAIAAVALLLKDADKLTLGQNVLVIAPHNLESIVRQPPD | |
| | RWMTNARMTHYQSLLLNERVSFAPPAILNPATLLPVESDDTPIHICSE | |
| | ILAEETGTRPDLRDQPLPGVPAWYTDGSSFIMDGRRQAGAAIVDNKRT | |
| | VWASNLPEGTSAQKAELIALTQALRLAEGKSINIYTDSRYAFATAHVH | |
| | GAIYKQRGWLTSAGKDIKNKEEILALLEAIHLPKRVAIIHCPGHQRGT | |
| | DPVATGNRKADEAAKQAAQSTRILTETTKN | |
| MLVAV_P03356 | TLNLEDEYRLYETSAEPEVSPGSTWLSDFPQAWAETGGMGLAVRQAPL | 380 |
| | IIPLKATSTPVSIKQYPMSQEAKLGIKPHIQRLLDQGILVPCQSPWNT | |
| | PLLPVKKPGTNDYRPVQDLREVNKRVEDIHPTVPNPYNLLSGLPPSHR | |
| | WYTVLDLKDAFFCLRLHPTSQPLFAFEWRDPGMGISGQLTWTRLPQGF | |
| | KNSPTLFDEALHRDLADFRIQHPDLILLQYVDDILLAATSELDCQQGT | |
| | RALLLTLGNLGYRASAKKAQLCQKQVKYLGYLLKEGQRWLTEARKETV | |
| | MGQPTPKTPRQLREFLGTAGFCRLWIPGFAEMAAPLYPLTKTGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLRKDAGKLTMGQPL | |
| | VILAPHAVEALVKQPPDRWLSNARMTHYQAMLLDTDRVQFGPVVALNP | |
| | ATLLPLPEEGAPHDCLEILAETHGTRPDLTDQPIPDADHTWYTDGSSF | |
| | LQEGQRKAGAAVTTETEVIWARALPAGTSAQRAELIALTQALKMAEGK | |
| | RLNVYTDSRYAFATAHIHGEIYRRRGLLTSEGREIKNKSEILALLKAL | |
| | FLPKRLSIIHCLGHQKGDSAEARGNRLADQAAREAAIKTPPDTSTLL | |
| MLVAV_P03356_ | TLNLEDEYRLYETSAEPEVSPGSTWLSDFPQAWAETGGMGLAVRQAPL | 381 |
| 3mut | IIPLKATSTPVSIKQYPMSQEAKLGIKPHIQRLLDQGILVPCQSPWNT | |
| | PLLPVKKPGTNDYRPVQDLREVNKRVEDIHPTVPNPYNLLSGLPPSHR | |
| | WYTVLDLKDAFFCLRLHPTSQPLFAFEWRDPGMGISGQLTWTRLPQGF | |
| | KNSPTLFNEALHRDLADFRIQHPDLILLQYVDDILLAATSELDCQQGT | |
| | RALLLTLGNLGYRASAKKAQLCQKQVKYLGYLLKEGQRWLTEARKETV | |
| | MGQPTPKTPRQLREFLGTAGFCRLWIPGFAEMAAPLYPLTKPGTLFNW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLRKDAGKLTMGQPL | |
| | VILAPHAVEALVKQPPDRWLSNARMTHYQAMLLDTDRVQFGPVVALNP | |
| | ATLLPLPEEGAPHDCLEILAETHGTRPDLTDQPIPDADHTWYTDGSSF | |
| | LQEGQRKAGAAVTTETEVIWARALPAGTSAQRAELIALTQALKMAEGK | |
| | RLNVYTDSRYAFATAHIHGEIYRRRGWLTSEGREIKNKSEILALLKAL | |
| | FLPKRLSIIHCLGHQKGDSAEARGNRLADQAAREAAIKTPPDTSTLL | |
| MLVAV_P03356_ | TLNLEDEYRLYETSAEPEVSPGSTWLSDFPQAWAETGGMGLAVRQAPL | 382 |
| 3mutA | IIPLKATSTPVSIKQYPMSQEAKLGIKPHIQRLLDQGILVPCQSPWNT | |
| | PLLPVKKPGTNDYRPVQDLREVNKRVEDIHPTVPNPYNLLSGLPPSHR | |
| | WYTVLDLKDAFFCLRLHPTSQPLFAFEWRDPGMGISGQLTWTRLPQGF | |
| | KNSPTLFNEALHRDLADFRIQHPDLILLQYVDDILLAATSELDCQQGT | |
| | RALLLTLGNLGYRASAKKAQLCQKQVKYLGYLLKEGQRWLTEARKETV | |
| | MGQPTPKTPRQLREFLGKAGFCRLFIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLRKDAGKLTMGQPL | |
| | VILAPHAVEALVKQPPDRWLSNARMTHYQAMLLDTDRVQFGPVVALNP | |
| | ATLLPLPEEGAPHDCLEILAETHGTRPDLTDQPIPDADHTWYTDGSSF | |
| | LQEGQRKAGAAVTTETEVIWARALPAGTSAQRAELIALTQALKMAEGK | |
| | RLNVYTDSRYAFATAHIHGEIYRRRGWLTSEGREIKNKSEILALLKAL | |
| | FLPKRLSIIHCLGHQKGDSAEARGNRLADQAAREAAIKTPPDTSTLL | |
| MLVBM_Q7SVK7 | TLGIEDEYRLHETSTEPDVSLGSTWLSDFPQAWAETGGMGLAVRQAPL | 383 |
| | IIPLKATSTPVSIQQYPMSHEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPGMGISGQLTWTRLPQGF | |
| | KNSPTLFDEALHRDLADFRIQHPDLILLQYVDDILLAATSELDCQQGT | |
| | RALLQTLGDLGYRASAKKAQICQKQVKYLGYLLREGQRWLTEARKETV | |
| | MGQPVPKTPRQLREFLGTAGFCRLWIPGFAEMAAPLYPLTKTGTLFSW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEEGAPHDCLEILAETHGTRPDLTDQPIPDADHTWYTDGSSF | |
| | LQEGQRKAGAAVTTETEVIWAGALPAGTSAQRAELIALTQALKMAEGK | |
| | RLNVYTDSRYAFATAHIHGEIYRRRGLLTSEGREIKNKSEILALLKAL | |
| | FLPKRLSIIHCLGHQKGDSAEARGNRLADQAAREAAIKTPPDTSTLL | |
| MLVBM_Q7SVK7 | TLGIEDEYRLHETSTEPDVSLGSTWLSDFPQAWAETGGMGLAVRQAPL | 384 |
| | IIPLKATSTPVSIQQYPMSHEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPGMGISGQLTWTRLPQGF | |
| | KNSPTLFDEALHRDLADFRIQHPDLILLQYVDDILLAATSELDCQQGT | |
| | RALLQTLGDLGYRASAKKAQICQKQVKYLGYLLREGQRWLTEARKETV | |
| | MGQPVPKTPRQLREFLGTAGFCRLWIPGFAEMAAPLYPLTKTGTLFSW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEEGAPHDCLEILAETHGTRPDLTDQPIPDADHTWYTDGSSF | |
| | LQEGQRKAGAAVTTETEVIWAGALPAGTSAQRAELIALTQALKMAEGK | |
| | RLNVYTDSRYAFATAHIHGEIYRRRGLLTSEGREIKNKSEILALLKAL | |
| | FLPKRLSIIHCLGHQKGDSAEARGNRLADQAAREAAIKTPPDTSTLL | |
| MLVBM_Q7SVK7_ | TLGIEDEYRLHETSTEPDVSLGSTWLSDFPQAWAETGGMGLAVRQAPL | 385 |
| 3mut | IIPLKATSTPVSIQQYPMSHEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPGMGISGQLTWTRLPQGF | |
| | KNSPTLFNEALHRDLADFRIQHPDLILLQYVDDILLAATSELDCQQGT | |
| | RALLQTLGDLGYRASAKKAQICQKQVKYLGYLLREGQRWLTEARKETV | |
| | MGQPVPKTPRQLREFLGTAGFCRLWIPGFAEMAAPLYPLTKPGTLESW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEEGAPHDCLEILAETHGTRPDLTDQPIPDADHTWYTDGSSF | |
| | LQEGQRKAGAAVTTETEVIWAGALPAGTSAQRAELIALTQALKMAEGK | |
| | RLNVYTDSRYAFATAHIHGEIYRRRGWLTSEGREIKNKSEILALLKAL | |
| | FLPKRLSIIHCLGHQKGDSAEARGNRLADQAAREAAIKTPPDTSTLL | |
| MLVBM_Q7SVK7_ | TLGIEDEYRLHETSTEPDVSLGSTWLSDFPQAWAETGGMGLAVRQAPL | 386 |
| 3mut | IIPLKATSTPVSIQQYPMSHEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPGMGISGQLTWTRLPQGF | |
| | KNSPTLFNEALHRDLADFRIQHPDLILLQYVDDILLAATSELDCQQGT | |
| | RALLQTLGDLGYRASAKKAQICQKQVKYLGYLLREGQRWLTEARKETV | |
| | MGQPVPKTPRQLREFLGTAGFCRLWIPGFAEMAAPLYPLTKPGTLFSW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEEGAPHDCLEILAETHGTRPDLTDQPIPDADHTWYTDGSSF | |
| | LQEGQRKAGAAVTTETEVIWAGALPAGTSAQRAELIALTQALKMAEGK | |
| | RLNVYTDSRYAFATAHIHGEIYRRRGWLTSEGREIKNKSEILALLKAL | |
| | FLPKRLSIIHCLGHQKGDSAEARGNRLADQAAREAAIKTPPDTSTLL | |
| MLVBM_Q7SVK7_ | LGIEDEYRLHETSTEPDVSLGSTWLSDFPQAWAETGGMGLAVRQAPLI | 387 |
| 3mutA_WS | IPLKATSTPVSIQQYPMSHEARLGIKPHIQRLLDQGILVPCQSPWNTP | |
| | LLPVKKPGTNDYRPVQDLREVNKRVEDIHPTVPNPYNLLSGLPPSHQW | |
| | YTVLDLKDAFFCLRLHPTSQPLFAFEWRDPGMGISGQLTWTRLPQGFK | |
| | NSPTLFNEALHRDLADFRIQHPDLILLQYVDDILLAATSELDCQQGTR | |
| | ALLQTLGDLGYRASAKKAQICQKQVKYLGYLLREGQRWLTEARKETVM | |
| | GQPVPKTPRQLREFLGKAGFCRLFIPGFAEMAAPLYPLTKPGTLFSWG | |
| | PDQQKAYQEIKQALLTAPALGLPDLTKPFELFVDEKQGYAKGVLTQKL | |
| | GPWRRPVAYLSKKLDPVAAGWPPCLRMVAAIAVLTKDAGKLTMGQPLV | |
| | ILAPHAVEALVKQPPDRWLSNARMTHYQAMLLDTDRVQFGPVVALNPA | |
| | TLLPLPEEGAPHDCLEILAETHGTRPDLTDQPIPDADHTWYTDGSSFL | |
| | QEGQRKAGAAVTTETEVIWAGALPAGTSAQRAELIALTQALKMAEGKR | |
| | LNVYTDSRYAFATAHIHGEIYRRRGWLTSEGREIKNKSEILALLKALF | |
| | LPKRLSIIHCLGHQKGDSAEARGNRLADQAAREAAIKTPPDTSTLLI | |
| MLVBM_Q7SVK7_ | LGIEDEYRLHETSTEPDVSLGSTWLSDFPQAWAETGGMGLAVRQAPLI | 388 |
| 3mutA_WS | IPLKATSTPVSIQQYPMSHEARLGIKPHIQRLLDQGILVPCQSPWNTP | |
| | LLPVKKPGTNDYRPVQDLREVNKRVEDIHPTVPNPYNLLSGLPPSHQW | |
| | YTVLDLKDAFFCLRLHPTSQPLFAFEWRDPGMGISGQLTWTRLPQGFK | |
| | NSPTLFNEALHRDLADFRIQHPDLILLQYVDDILLAATSELDCQQGTR | |
| | ALLQTLGDLGYRASAKKAQICQKQVKYLGYLLREGQRWLTEARKETVM | |
| | GQPVPKTPRQLREFLGKAGFCRLFIPGFAEMAAPLYPLTKPGTLFSWG | |
| | PDQQKAYQEIKQALLTAPALGLPDLTKPFELFVDEKQGYAKGVLTQKL | |
| | GPWRRPVAYLSKKLDPVAAGWPPCLRMVAAIAVLTKDAGKLTMGQPLV | |
| | ILAPHAVEALVKQPPDRWLSNARMTHYQAMLLDTDRVQFGPVVALNPA | |
| | TLLPLPEEGAPHDCLEILAETHGTRPDLTDQPIPDADHTWYTDGSSEL | |
| | QEGQRKAGAAVTTETEVIWAGALPAGTSAQRAELIALTQALKMAEGKR | |
| | LNVYTDSRYAFATAHIHGEIYRRRGWLISEGREIKNKSEILALLKALF | |
| | LPKRLSIIHCLGHQKGDSAEARGNRLADQAAREAAIKTPPDTSTLLI | |
| MLVCB_P08361 | TLNIEDEYRLHETSKEPDVSLGSTWLSDFPQAWAETGGMGLAVRQAPL | 389 |
| | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFDEALHRDLAGFRIQHPDLILLQYVDDLLLAATSELDCQQGT | |
| | RALLQTLGDLGYRASAKKAQICQKQVKYLGYLLKEGQRWLTEARKETV | |
| | MGQPIPKTPRQLREFLGTAGFCRLWIPGFAEMAAPLYPLTKTGTLFNW | |
| | GPDQQKAFQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHDCLDILAEAHGTRSDLMDQPLPDADHTWYTDGSSF | |
| | LQEGQRKAGAAVTTETEVIWARALPAGTSAQRAELIALTQALKMAEGK | |
| | KLNVYTDSRYAFATAHIHGEIYRRRGLLTSEGKEIKNKDEILALLKAL | |
| | FLPKRLSIIHCPGHQKGNSAEARGNRMADQAAREVATRETPETSTLL | |
| MLVCB_P08361_ | TLNIEDEYRLHETSKEPDVSLGSTWLSDFPQAWAETGGMGLAVRQAPL | 390 |
| 3mut | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLAGFRIQHPDLILLQYVDDLLLAATSELDCQQGT | |
| | RALLQTLGDLGYRASAKKAQICQKQVKYLGYLLKEGQRWLTEARKETV | |
| | MGQPIPKTPRQLREFLGTAGFCRLWIPGFAEMAAPLYPLTKPGTLENW | |
| | GPDQQKAFQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHDCLDILAEAHGTRSDLMDQPLPDADHTWYTDGSSF | |
| | LQEGQRKAGAAVTTETEVIWARALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGNSAEARGNRMADQAAREVATRETPETSTLL | |
| MLVCB_P08361_ | TLNIEDEYRLHETSKEPDVSLGSTWLSDFPQAWAETGGMGLAVRQAPL | 391 |
| 3mutA | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLAGFRIQHPDLILLQYVDDLLLAATSELDCQQGT | |
| | RALLQTLGDLGYRASAKKAQICQKQVKYLGYLLKEGQRWLTEARKETV | |
| | MGQPIPKTPRQLREFLGKAGFCRLFIPGFAEMAAPLYPLTKPGTLFNW | |
| | GPDQQKAFQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHDCLDILAEAHGTRSDLMDQPLPDADHTWYTDGSSF | |
| | LQEGQRKAGAAVTTETEVIWARALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGNSAEARGNRMADQAAREVATRETPETSTLL | |
| MLVF5_P26810 | TLNIEDEYRLHETSKGPDVPLGSTWLSDFPQAWAETGGMGLAFRQAPL | 392 |
| | IISLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQSLFAFEWKDPEMGISGQLTWTRLPQGF | |
| | KNSPTLFDEALHRDLADFRIQHPDLILLQYVDDLLLAATSELDCQQGT | |
| | RALLQTLGDLGYRASAKKAQICQKQVKYLGYLLKEGQRWLTEARKETV | |
| | MGQPTPKTPRQLREFLGTAGLCRLWIPGFAEMAAPLYPLTKTGTLFKW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDVGKLTMGQPL | |
| | VILAPHAVEALVKQPPDRWLSNARMTHYQALLLDTDRVQFGPIVALNP | |
| | ATLLPLPEEGLQHDCLDILAEAHGTRPDLTDQPLPDADHTWYTDGSSF | |
| | LQEGQRRAGAAVTTETEVIWAKALPAGTSAQRAELIALTQALKMAAGK | |
| | KLNVYTDSRYAFATAHIHGEIYRRRGLLTSEGKEIKNKDEILALLKAL | |
| | FLPKRLSIIHCPGHQKGNHAEARGNRMADQAAREVATRETPETSTLL | |
| MLVF5_P26810_ | TLNIEDEYRLHETSKGPDVPLGSTWLSDFPQAWAETGGMGLAFRQAPL | 393 |
| 3mut | IISLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQSLFAFEWKDPEMGISGQLTWTRLPQGF | |
| | KNSPTLFNEALHRDLADFRIQHPDLILLQYVDDLLLAATSELDCQQGT | |
| | RALLQTLGDLGYRASAKKAQICQKQVKYLGYLLKEGQRWLTEARKETV | |
| | MGQPTPKTPRQLREFLGTAGLCRLWIPGFAEMAAPLYPLTKPGTLFKW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDVGKLTMGQPL | |
| | VILAPHAVEALVKQPPDRWLSNARMTHYQALLLDTDRVQFGPIVALNP | |
| | ATLLPLPEEGLQHDCLDILAEAHGTRPDLTDQPLPDADHTWYTDGSSF | |
| | LQEGQRRAGAAVTTETEVIWAKALPAGTSAQRAELIALTQALKMAAGK | |
| | KLNVYTDSRYAFATAHIHGEIYRRRGWLTSEGKEIKNKDEILALLKAL | |
| | FLPKRLSIIHCPGHQKGNHAEARGNRMADQAAREVATRETPETSTLL | |
| MLVF5_P26810_ | TLNIEDEYRLHETSKGPDVPLGSTWLSDFPQAWAETGGMGLAFRQAPL | 394 |
| 3mutA | IISLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQSLFAFEWKDPEMGISGQLTWIRLPQGF | |
| | KNSPTLFNEALHRDLADFRIQHPDLILLQYVDDLLLAATSELDCQQGT | |
| | RALLQTLGDLGYRASAKKAQICQKQVKYLGYLLKEGQRWLTEARKETV | |
| | MGQPTPKTPRQLREFLGKAGLCRLFIPGFAEMAAPLYPLTKPGTLFKW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDVGKLTMGQPL | |
| | VILAPHAVEALVKQPPDRWLSNARMTHYQALLLDTDRVQFGPIVALNP | |
| | ATLLPLPEEGLQHDCLDILAEAHGTRPDLTDQPLPDADHTWYTDGSSF | |
| | LQEGQRRAGAAVTTETEVIWAKALPAGTSAQRAELIALTQALKMAAGK | |
| | KLNVYTDSRYAFATAHIHGEIYRRRGWLTSEGKEIKNKDEILALLKAL | |
| | FLPKRLSIIHCPGHQKGNHAEARGNRMADQAAREVATRETPETSTLL | |
| MLVFF_P26809_ | TLNIEDEYRLHETSKGPDVPLGSTWLSDFPQAWAETGGMGLAVRQAPL | 395 |
| 3mut | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQSLFAFEWRDPEMGISGQLTWTRLPQGF | |
| | KNSPTLFNEALHRDLADFRIQHPDLILLQYVDDLLLAATSELDCQQGT | |
| | RALLQTLGDLGYRASAKKAQICQKQVKYLGYLLKEGQRWLTEARKETV | |
| | MGQPTPKTPRQLREFLGTAGFCRLWIPGFAEMAAPLYPLTKPGTLFEW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPIVALNP | |
| | ATLLPLPEEGLQHDCLDILAEAHGTRPDLTDQPLPDADHTWYTDGSSF | |
| | LQEGQRKAGAAVTTETEVVWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGNRAEARGNRMADQAAREVATRETPETSTLL | |
| MLVFF_P26809_ | TLNIEDEYRLHETSKGPDVPLGSTWLSDFPQAWAETGGMGLAVRQAPL | 396 |
| 3mutA | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQSLFAFEWRDPEMGISGQLTWTRLPQGF | |
| | KNSPTLFNEALHRDLADFRIQHPDLILLQYVDDLLLAATSELDCQQGT | |
| | RALLQTLGDLGYRASAKKAQICQKQVKYLGYLLKEGQRWLTEARKETV | |
| | MGQPTPKTPRQLREFLGKAGFCRLFIPGFAEMAAPLYPLTKPGTLFEW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPIVALNP | |
| | ATLLPLPEEGLQHDCLDILAEAHGTRPDLTDQPLPDADHTWYTDGSSF | |
| | LQEGQRKAGAAVTTETEVVWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGNRAEARGNRMADQAAREVATRETPETSTLL | |
| MLVMS_P03355 | TLNIEDEHRLHETSKEPDVSLGSTWLSDFPQAWAETGGMGLAVRQAPL | 397 |
| | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFDEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGTAGFCRLWIPGFAEMAAPLYPLTKTGTLFNW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLL | |
| MLVMS_reference | TLNIEDEYRLHETSKEPDVSLGSTWLSDFPQAWAETGGMGLAVRQAPL | 398 |
| | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGKAGFCRLFIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGHSAEARGNRMADQAARKAAITETPDTSTLLI | |
| | ENSSP | |
| MLVMS_P03355 | TLNIEDEHRLHETSKEPDVSLGSTWLSDFPQAWAETGGMGLAVRQAPL | 399 |
| | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFDEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGTAGFCRLWIPGFAEMAAPLYPLTKTGTLFNW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGLLTSEGKEIKNKDEILALLKAL | |
| | FLPKRLSIIHCPGHQKGHSAEARGNRMADQAARKAAITETPDTSTLL | |
| MLVMS_P03355_ | TLNIEDEHRLHETSKEPDVSLGSTWLSDFPQAWAETGGMGLAVRQAPL | 400 |
| 3mut | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGTAGFCRLWIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGHSAEARGNRMADQAARKAAITETPDTSTLL | |
| MLVMS_P03355_ | TLNIEDEHRLHETSKEPDVSLGSTWLSDFPQAWAETGGMGLAVRQAPL | 401 |
| 3mut | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGTAGFCRLWIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGHSAEARGNRMADQAARKAAITETPDTSTLL | |
| MLVMS_P03355_ | TLNIEDEHRLHETSKEPDVSLGSTWLSDFPQAWAETGGMGLAVRQAPL | 402 |
| 3mutA_WS | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGKAGFCRLFIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGHSAEARGNRMADQAARKAAITETPDTSTLL | |
| MLVMS_P03355_ | TLNIEDEHRLHETSKEPDVSLGSTWLSDFPQAWAETGGMGLAVRQAPL | 403 |
| 3mutA_WS | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWIRLPQGF | |
| | KNSPTLFNEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGKAGFCRLFIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGHSAEARGNRMADQAARKAAITETPDTSTLL | |
| MLVMS_P03355_ | TLNIEDEYRLHETSKEPDVSLGSTWLSDFPQAWAETGGMGLAVRQAPL | 404 |
| PLV919 | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWIRLPQGF | |
| | KNSPTLFNEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGKAGFCRLFIPGFAEMAAPLYPLTKPGTLFNW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGHSAEARGNRMADQAARKAAITETPDTSTLLI | |
| | ENSSPSGGSKRTADGSEFE | |
| MLVMS_P03355_ | TLNIEDEYRLHETSKEPDVSLGSTWLSDFPQAWAETGGMGLAVRQAPL | 405 |
| PLV919 | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLADFRIQHPDLILLQYVDDLLLAATSELDCQQGT | |
| | RALLQTLGNLGYRASAKKAQICQKQVKYLGYLLKEGQRWLTEARKETV | |
| | MGQPTPKTPRQLREFLGKAGFCRLFIPGFAEMAAPLYPLTKPGTLFNW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQALLLDTDRVQFGPVVALNP | |
| | ATLLPLPEEGLQHNCLDILAEAHGTRPDLTDQPLPDADHTWYTDGSSL | |
| | LQEGQRKAGAAVTTETEVIWAKALPAGTSAQRAELIALTQALKMAEGK | |
| | KLNVYTDSRYAFATAHIHGEIYRRRGWLTSEGKEIKNKDEILALLKAL | |
| | FLPKRLSIIHCPGHQKGHSAEARGNRMADQAARKAAITETPDTSTLLI | |
| | ENSSPSGGSKRTADGSEFE | |
| MLVRD_P11227 | TLNIEDEYRLHEISTEPDVSPGSTWLSDFPQAWAETGGMGLAVRQAPL | 406 |
| | IIPLKATSTPVSIKQYPMSQEAKLGIKPHIQRLLDQGILVPCQSPWNT | |
| | PLLPVKKPGTNDYRPVQGLREVNKRVEDIHPTVPNPYNLLSGLPTSHR | |
| | WYTVLDLKDAFFCLRLHPTSQPLFASEWRDPGMGISGQLTWTRLPQGF | |
| | KNSPTLFDEALHRGLADFRIQHPDLILLQYVDDLLLAATSELDCQQGT | |
| | RALLKTLGNLGYRASAKKAQICQKQVKYLGYLLREGQRWLTEARKETV | |
| | MGQPTPKTPRQLREFLGTAGFCRLWIPRFAEMAAPLYPLTKTGTLFNW | |
| | GPDQQKAYHEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEEGAPHDCLEILAETHGTEPDLTDQPIPDADHTWYTDGSSF | |
| | LQEGQRKAGAAVTTETEVIWARALPAGTSAQRAELIALTQALKMAEGK | |
| | RLNVYTDSRYAFATAHIHGEIYKRRGLLTSEGREIKNKSEILALLKAL | |
| | FLPKRLSIIHCLGHQKGDSAEARGNRLADQAAREAAIKTPPDTSTLL | |
| MLVRD_P11227_ | TLNIEDEYRLHEISTEPDVSPGSTWLSDFPQAWAETGGMGLAVRQAPL | 407 |
| 3mut | IIPLKATSTPVSIKQYPMSQEAKLGIKPHIQRLLDQGILVPCQSPWNT | |
| | PLLPVKKPGTNDYRPVQGLREVNKRVEDIHPTVPNPYNLLSGLPTSHR | |
| | WYTVLDLKDAFFCLRLHPTSQPLFASEWRDPGMGISGQLTWTRLPQGF | |
| | KNSPTLFNEALHRGLADFRIQHPDLILLQYVDDLLLAATSELDCQQGT | |
| | RALLKTLGNLGYRASAKKAQICQKQVKYLGYLLREGQRWLTEARKETV | |
| | MGQPTPKTPRQLREFLGTAGFCRLWIPRFAEMAAPLYPLTKPGTLENW | |
| | GPDQQKAYHEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEEGAPHDCLEILAETHGTEPDLTDQPIPDADHTWYTDGSSF | |
| | LQEGQRKAGAAVTTETEVIWARALPAGTSAQRAELIALTQALKMAEGK | |
| | RLNVYTDSRYAFATAHIHGEIYKRRGWLTSEGREIKNKSEILALLKAL | |
| | FLPKRLSIIHCLGHQKGDSAEARGNRLADQAAREAAIKTPPDTSTLL | |
| MMTVB_P03365 | WVQEISDSRPMLHIYLNGRRFLGLLNTGADKTCIAGRDWPANWPIHQT | 408 |
| | ESSLQGLGMACGVARSSQPLRWQHEDKSGIIHPFVIPTLPFTLWGRDI | |
| | MKDIKVRLMTDSPDDSQDLMIGAIESNLFADQISWKSDQPVWLNQWPL | |
| | KQEKLQALQQLVTEQLQLGHLEESNSPWNTPVFVIKKKSGKWRLLQDL | |
| | RAVNATMHDMGALQPGLPSPVAVPKGWEIIIIDLQDCFFNIKLHPEDC | |
| | KRFAFSVPSPNFKRPYQRFQWKVLPQGMKNSPTLCQKFVDKAILTVRD | |
| | KYQDSYIVHYMDDILLAHPSRSIVDEILTSMIQALNKHGLVVSTEKIQ | |
| | KYDNLKYLGTHIQGDSVSYQKLQIRTDKLRTLNDFQKLLGNINWIRPF | |
| | LKLTTGELKPLFEILNGDSNPISTRKLTPEACKALQLMNERLSTARVK | |
| | RLDLSQPWSLCILKTEYTPTACLWQDGVVEWIHLPHISPKVITPYDIF | |
| | CTQLIIKGRHRSKELFSKDPDYIVVPYTKVQFDLLLQEKEDWPISLLG | |
| | FLGEVHFHLPKDPLLTFTLQTAIIFPHMTSTTPLEKGIVIFTDGSANG | |
| | RSVTYIQGREPIIKENTQNTAQQAEIVAVITAFEEVSQPENLYTDSKY | |
| | VTGLFPEIETATLSPRTKIYTELKHLQRLIHKRQEKFYIGHIRGHTGL | |
| | PGPLAQGNAYADSLTRILT | |
| MMTVB_P03365 | WVQEISDSRPMLHIYLNGRRFLGLLNTGADKTCIAGRDWPANWPIHQT | 409 |
| | ESSLQGLGMACGVARSSQPLRWQHEDKSGIIHPFVIPTLPFTLWGRDI | |
| | MKDIKVRLMTDSPDDSQDLMIGAIESNLFADQISWKSDQPVWLNQWPL | |
| | KQEKLQALQQLVTEQLQLGHLEESNSPWNTPVFVIKKKSGKWRLLQDL | |
| | RAVNATMHDMGALQPGLPSPVAVPKGWEIIIIDLQDCFFNIKLHPEDC | |
| | KRFAFSVPSPNFKRPYQRFQWKVLPQGMKNSPTLCQKFVDKAILTVRD | |
| | KYQDSYIVHYMDDILLAHPSRSIVDEILTSMIQALNKHGLVVSTEKIQ | |
| | KYDNLKYLGTHIQGDSVSYQKLQIRTDKLRTLNDFQKLLGNINWIRPF | |
| | LKLTTGELKPLFEILNGDSNPISTRKLTPEACKALQLMNERLSTARVK | |
| | RLDLSQPWSLCILKTEYTPTACLWQDGVVEWIHLPHISPKVITPYDIF | |
| | CTQLIIKGRHRSKELFSKDPDYIVVPYTKVQFDLLLQEKEDWPISLLG | |
| | FLGEVHFHLPKDPLLTFTLQTAIIFPHMTSTTPLEKGIVIFTDGSANG | |
| | RSVTYIQGREPIIKENTQNTAQQAEIVAVITAFEEVSQPENLYTDSKY | |
| | VTGLFPEIETATLSPRTKIYTELKHLQRLIHKRQEKFYIGHIRGHTGL | |
| | PGPLAQGNAYADSLTRILT | |
| MMTVB_P03365_ | WVQEISDSRPMLHIYLNGRRFLGLLNTGADKTCIAGRDWPANWPIHQT | 410 |
| 2mut | ESSLQGLGMACGVARSSQPLRWQHEDKSGIIHPFVIPTLPFTLWGRDI | |
| | MKDIKVRLMTDSPDDSQDLMIGAIESNLFADQISWKSDQPVWLNQWPL | |
| | KQEKLQALQQLVTEQLQLGHLEESNSPWNTPVFVIKKKSGKWRLLQDL | |
| | RAVNATMHDMGALQPGLPSPVAVPKGWEIIIIDLQDCFFNIKLHPEDC | |
| | KRFAFSVPSPNFKRPYQRFQWKVLPQGMKNSPTLCQKFVDKAILTVRD | |
| | KYQDSYIVHYMDDILLAHPSRSIVDEILTSMIQALNKHGLVVSTEKIQ | |
| | KYDNLKYLGTHIQGDSVSYQKLQIRTDKLRTLNDFQKLLGNINWIRPF | |
| | LKLTTGELKPLFEILNPDSNPISTRKLTPEACKALQLMNERLSTARVK | |
| | RLDLSQPWSLCILKTEYTPTACLWQDGVVEWIHLPHISPKVITPYDIF | |
| | CTQLIIKGRHRSKELFSKDPDYIVVPYTKVQFDLLLQEKEDWPISLLG | |
| | FLGEVHFHLPKDPLLTFTLQTAIIFPHMTSTTPLEKGIVIFTDGSANG | |
| | RSVTYIQGREPIIKENTQNTAQQAEIVAVITAFEEVSQPFNLYTDSKY | |
| | VTGLFPEIETATLSPRTKIYTELKHLQRLIHKRQEKFYIGHIRGHTGL | |
| | PGPLAQGNAYADSLTRILT | |
| MMTVB_P03365_ | VQEISDSRPMLHIYLNGRRFLGLLDTGADKTCIAGRDWPANWPIHQTE | 411 |
| 2mut_WS | SSLQGLGMACGVARSSQPLRWQHEDKSGIIHPFVIPTLPFTLWGRDIM | |
| | KDIKVRLMTDSPDDSQDLMIGAIESNLFADQISWKSDQPVWLNQWPLK | |
| | QEKLQALQQLVTEQLQLGHLEESNSPWNTPVFVIKKKSGKWRLLQDLR | |
| | AVNATMHDMGALQPGLPSPVAVPKGWEIIIIDLQDCFFNIKLHPEDCK | |
| | RFAFSVPSPNFKRPYQRFQWKVLPQGMKNSPTLCQKFVDKAILTVRDK | |
| | YQDSYIVHYMDDILLAHPSRSIVDEILTSMIQALNKHGLVVSTEKIQK | |
| | YDNLKYLGTHIQGDSVSYQKLQIRTDKLRTLNDFQKLLGNINWIRPFL | |
| | KLTTGELKPLFEILNPDSNPISTRKLTPEACKALQLMNERLSTARVKR | |
| | LDLSQPWSLCILKTEYTPTACLWQDGVVEWIHLPHISPKVITPYDIFC | |
| | TQLIIKGRHRSKELFSKDPDYIVVPYTKVQFDLLLQEKEDWPISLLGF | |
| | LGEVHFHLPKDPLLTFTLQTAIIFPHMTSTTPLEKGIVIFTDGSANGR | |
| | SVTYIQGREPIIKENTQNTAQQAEIVAVITAFEEVSQPENLYTDSKYV | |
| | TGLFPEIETATLSPRTKIYTELKHLQRLIHKRQEKFYIGHIRGHTGLP | |
| | GPLAQGNAYADSLTRILTA | |
| MMTVB_P03365_ | VQEISDSRPMLHIYLNGRRFLGLLDTGADKTCIAGRDWPANWPIHQTE | 412 |
| 2mut_WS | SSLQGLGMACGVARSSQPLRWQHEDKSGIIHPFVIPTLPFTLWGRDIM | |
| | KDIKVRLMTDSPDDSQDLMIGAIESNLFADQISWKSDQPVWLNQWPLK | |
| | QEKLQALQQLVTEQLQLGHLEESNSPWNTPVFVIKKKSGKWRLLQDLR | |
| | AVNATMHDMGALQPGLPSPVAVPKGWEIIIIDLQDCFFNIKLHPEDCK | |
| | RFAFSVPSPNFKRPYQRFQWKVLPQGMKNSPTLCQKFVDKAILTVRDK | |
| | YQDSYIVHYMDDILLAHPSRSIVDEILTSMIQALNKHGLVVSTEKIQK | |
| | YDNLKYLGTHIQGDSVSYQKLQIRTDKLRTLNDFQKLLGNINWIRPFL | |
| | KLTTGELKPLFEILNPDSNPISTRKLTPEACKALQLMNERLSTARVKR | |
| | LDLSQPWSLCILKTEYTPTACLWQDGVVEWIHLPHISPKVITPYDIFC | |
| | TQLIIKGRHRSKELFSKDPDYIVVPYTKVQFDLLLQEKEDWPISLLGF | |
| | LGEVHFHLPKDPLLTFTLQTAIIFPHMTSTTPLEKGIVIFTDGSANGR | |
| | SVTYIQGREPIIKENTQNTAQQAEIVAVITAFEEVSQPENLYTDSKYV | |
| | TGLFPEIETATLSPRTKIYTELKHLQRLIHKRQEKFYIGHIRGHTGLP | |
| | GPLAQGNAYADSLTRILTA | |
| MMTVB_P03365_ | WVQEISDSRPMLHIYLNGRRFLGLLNTGADKTCIAGRDWPANWPIHQT | 413 |
| 2mutB | ESSLQGLGMACGVARSSQPLRWQHEDKSGIIHPFVIPTLPFTLWGRDI | |
| | MKDIKVRLMTDSPDDSQDLMIGAIESNLFADQISWKSDQPVWLNQWPL | |
| | KQEKLQALQQLVTEQLQLGHLEESNSPWNTPVFVIKKKSGKWRLLQDL | |
| | RAVNATMHDMGALQPGLPSPVAPPKGWEIIIIDLQDCFFNIKLHPEDC | |
| | KRFAFSVPSPNFKRPYQRFQWKVLPQGMKNSPTLCQKFVDKAILTVRD | |
| | KYQDSYIVHYMDDILLAHPSRSIVDEILTSMIQALNKHGLVVSTEKIQ | |
| | KYDNLKYLGTHIQGDSVSYQKLQIRTDKLRTLNDFQKLLGNINWIRPF | |
| | LKLTTGELKPLFEILNPDSNPISTRKLTPEACKALQLMNERLSTARVK | |
| | RLDLSQPWSLCILKTEYTPTACLWQDGVVEWIHLPHISPKVITPYDIF | |
| | CTQLIIKGRHRSKELFSKDPDYIVVPYTKVQFDLLLQEKEDWPISLLG | |
| | FLGEVHFHLPKDPLLTFTLQTAIIFPHMTSTTPLEKGIVIFTDGSANG | |
| | RSVTYIQGREPIIKENTQNTAQQAEIVAVITAFEEVSQPFNLYTDSKY | |
| | VTGLFPEIETATLSPRTKIYTELKHLQRLIHKRQEKFYIGHIRGHTGL | |
| | PGPLAQGNAYADSLTRILT | |
| MMTVB_P03365_ | WVQEISDSRPMLHIYLNGRRFLGLLNTGADKTCIAGRDWPANWPIHQT | 414 |
| 2mutB | ESSLQGLGMACGVARSSQPLRWQHEDKSGIIHPFVIPTLPFTLWGRDI | |
| | MKDIKVRLMTDSPDDSQDLMIGAIESNLFADQISWKSDQPVWLNQWPL | |
| | KQEKLQALQQLVTEQLQLGHLEESNSPWNTPVFVIKKKSGKWRLLQDL | |
| | RAVNATMHDMGALQPGLPSPVAPPKGWEIIIIDLQDCFFNIKLHPEDC | |
| | KRFAFSVPSPNFKRPYQRFQWKVLPQGMKNSPTLCQKFVDKAILTVRD | |
| | KYQDSYIVHYMDDILLAHPSRSIVDEILTSMIQALNKHGLVVSTEKIQ | |
| | KYDNLKYLGTHIQGDSVSYQKLQIRTDKLRTLNDFQKLLGNINWIRPF | |
| | LKLTTGELKPLFEILNPDSNPISTRKLTPEACKALQLMNERLSTARVK | |
| | RLDLSQPWSLCILKTEYTPTACLWQDGVVEWIHLPHISPKVITPYDIF | |
| | CTQLIIKGRHRSKELFSKDPDYIVVPYTKVQFDLLLQEKEDWPISLLG | |
| | FLGEVHFHLPKDPLLTFTLQTAIIFPHMTSTTPLEKGIVIFTDGSANG | |
| | RSVTYIQGREPIIKENTQNTAQQAEIVAVITAFEEVSQPFNLYTDSKY | |
| | VTGLFPEIETATLSPRTKIYTELKHLQRLIHKRQEKFYIGHIRGHTGL | |
| | PGPLAQGNAYADSLTRILT | |
| MMTVB_P03365_ | VQEISDSRPMLHIYLNGRRFLGLLDTGADKTCIAGRDWPANWPIHQTE | 415 |
| 2mutB_WS | SSLQGLGMACGVARSSQPLRWQHEDKSGIIHPFVIPTLPFTLWGRDIM | |
| | KDIKVRLMTDSPDDSQDLMIGAIESNLFADQISWKSDQPVWLNQWPLK | |
| | QEKLQALQQLVTEQLQLGHLEESNSPWNTPVFVIKKKSGKWRLLQDLR | |
| | AVNATMHDMGALQPGLPSPPAVPKGWEIIIIDLQDCFFNIKLHPEDCK | |
| | RFAFSVPSPNFKRPYQRFQWKVLPQGMKNSPTLCQKFVDKAILTVRDK | |
| | YQDSYIVHYMDDILLAHPSRSIVDEILTSMIQALNKHGLVVSTEKIQK | |
| | YDNLKYLGTHIQGDSVSYQKLQIRTDKLRTLNDFQKLLGNINWIRPFL | |
| | KLTTGELKPLFEILNPDSNPISTRKLTPEACKALQLMNERLSTARVKR | |
| | LDLSQPWSLCILKTEYTPTACLWQDGVVEWIHLPHISPKVITPYDIFC | |
| | TQLIIKGRHRSKELFSKDPDYIVVPYTKVQFDLLLQEKEDWPISLLGF | |
| | LGEVHFHLPKDPLLTFTLQTAIIFPHMTSTTPLEKGIVIFTDGSANGR | |
| | SVTYIQGREPIIKENTQNTAQQAEIVAVITAFEEVSQPFNLYTDSKYV | |
| | TGLFPEIETATLSPRTKIYTELKHLQRLIHKRQEKFYIGHIRGHTGLP | |
| | GPLAQGNAYADSLTRILTA | |
| MMTVB_P03365_ | VQEISDSRPMLHIYLNGRRFLGLLDTGADKTCIAGRDWPANWPIHQTE | 416 |
| 2mutB_WS | SSLQGLGMACGVARSSQPLRWQHEDKSGIIHPFVIPTLPFTLWGRDIM | |
| | KDIKVRLMTDSPDDSQDLMIGAIESNLFADQISWKSDQPVWLNQWPLK | |
| | QEKLQALQQLVTEQLQLGHLEESNSPWNTPVFVIKKKSGKWRLLQDLR | |
| | AVNATMHDMGALQPGLPSPPAVPKGWEIIIIDLQDCFFNIKLHPEDCK | |
| | RFAFSVPSPNFKRPYQRFQWKVLPQGMKNSPTLCQKFVDKAILTVRDK | |
| | YQDSYIVHYMDDILLAHPSRSIVDEILTSMIQALNKHGLVVSTEKIQK | |
| | YDNLKYLGTHIQGDSVSYQKLQIRTDKLRTLNDFQKLLGNINWIRPFL | |
| | KLTTGELKPLFEILNPDSNPISTRKLTPEACKALQLMNERLSTARVKR | |
| | LDLSQPWSLCILKTEYTPTACLWQDGVVEWIHLPHISPKVITPYDIFC | |
| | TQLIIKGRHRSKELFSKDPDYIVVPYTKVQFDLLLQEKEDWPISLLGF | |
| | LGEVHFHLPKDPLLTFTLQTAIIFPHMTSTTPLEKGIVIFTDGSANGR | |
| | SVTYIQGREPIIKENTQNTAQQAEIVAVITAFEEVSQPFNLYTDSKYV | |
| | TGLFPEIETATLSPRTKIYTELKHLQRLIHKRQEKFYIGHIRGHTGLP | |
| | GPLAQGNAYADSLTRILTA | |
| MMTVB_P03365_ | VQEISDSRPMLHIYLNGRRFLGLLDTGADKTCIAGRDWPANWPIHQTE | 417 |
| WS | SSLQGLGMACGVARSSQPLRWQHEDKSGIIHPFVIPTLPFTLWGRDIM | |
| | KDIKVRLMTDSPDDSQDLMIGAIESNLFADQISWKSDQPVWLNQWPLK | |
| | QEKLQALQQLVTEQLQLGHLEESNSPWNTPVFVIKKKSGKWRLLQDLR | |
| | AVNATMHDMGALQPGLPSPVAVPKGWEIIIIDLQDCFFNIKLHPEDCK | |
| | RFAFSVPSPNFKRPYQRFQWKVLPQGMKNSPTLCQKFVDKAILTVRDK | |
| | YQDSYIVHYMDDILLAHPSRSIVDEILTSMIQALNKHGLVVSTEKIQK | |
| | YDNLKYLGTHIQGDSVSYQKLQIRTDKLRTLNDFQKLLGNINWIRPFL | |
| | KLTTGELKPLFEILNGDSNPISTRKLTPEACKALQLMNERLSTARVKR | |
| | LDLSQPWSLCILKTEYTPTACLWQDGVVEWIHLPHISPKVITPYDIFC | |
| | TQLIIKGRHRSKELFSKDPDYIVVPYTKVQFDLLLQEKEDWPISLLGF | |
| | LGEVHFHLPKDPLLTFTLQTAIIFPHMTSTTPLEKGIVIFTDGSANGR | |
| | SVTYIQGREPIIKENTQNTAQQAEIVAVITAFEEVSQPFNLYTDSKYV | |
| | TGLFPEIETATLSPRTKIYTELKHLQRLIHKRQEKFYIGHIRGHTGLP | |
| | GPLAQGNAYADSLTRILTA | |
| MMTVB_P03365_ | VQEISDSRPMLHIYLNGRRFLGLLDTGADKTCIAGRDWPANWPIHQTE | 418 |
| WS | SSLQGLGMACGVARSSQPLRWQHEDKSGIIHPFVIPTLPFTLWGRDIM | |
| | KDIKVRLMTDSPDDSQDLMIGAIESNLFADQISWKSDQPVWLNQWPLK | |
| | QEKLQALQQLVTEQLQLGHLEESNSPWNTPVFVIKKKSGKWRLLQDLR | |
| | AVNATMHDMGALQPGLPSPVAVPKGWEIIIIDLQDCFFNIKLHPEDCK | |
| | RFAFSVPSPNFKRPYQRFQWKVLPQGMKNSPTLCQKFVDKAILTVRDK | |
| | YQDSYIVHYMDDILLAHPSRSIVDEILTSMIQALNKHGLVVSTEKIQK | |
| | YDNLKYLGTHIQGDSVSYQKLQIRTDKLRTLNDFQKLLGNINWIRPFL | |
| | KLTTGELKPLFEILNGDSNPISTRKLTPEACKALQLMNERLSTARVKR | |
| | LDLSQPWSLCILKTEYTPTACLWQDGVVEWIHLPHISPKVITPYDIFC | |
| | TQLIIKGRHRSKELFSKDPDYIVVPYTKVQFDLLLQEKEDWPISLLGE | |
| | LGEVHFHLPKDPLLTFTLQTAIIFPHMTSTTPLEKGIVIFTDGSANGR | |
| | SVTYIQGREPIIKENTQNTAQQAEIVAVITAFEEVSQPFNLYTDSKYV | |
| | TGLFPEIETATLSPRTKIYTELKHLQRLIHKRQEKFYIGHIRGHTGLP | |
| | GPLAQGNAYADSLTRILTA | |
| MMTVB_P03365- | GRDIMKDIKVRLMTDSPDDSQDLMIGAIESNLFADQISWKSDQPVWLN | 419 |
| Pro | QWPLKQEKLQALQQLVTEQLQLGHLEESNSPWNTPVFVIKKKSGKWRL | |
| | LQDLRAVNATMHDMGALQPGLPSPVAVPKGWEIIIIDLQDCFFNIKLH | |
| | PEDCKRFAFSVPSPNFKRPYQRFQWKVLPQGMKNSPTLCQKFVDKAIL | |
| | TVRDKYQDSYIVHYMDDILLAHPSRSIVDEILTSMIQALNKHGLVVST | |
| | EKIQKYDNLKYLGTHIQGDSVSYQKLQIRTDKLRTLNDFQKLLGNINW | |
| | IRPFLKLTTGELKPLFEILNGDSNPISTRKLTPEACKALQLMNERLST | |
| | ARVKRLDLSQPWSLCILKTEYTPTACLWQDGVVEWIHLPHISPKVITP | |
| | YDIFCTQLIIKGRHRSKELFSKDPDYIVVPYTKVQFDLLLQEKEDWPI | |
| | SLLGFLGEVHFHLPKDPLLTFTLQTAIIFPHMTSTTPLEKGIVIFTDG | |
| | SANGRSVTYIQGREPIIKENTQNTAQQAEIVAVITAFEEVSQPENLYT | |
| | DSKYVTGLFPEIETATLSPRTKIYTELKHLQRLIHKRQEKFYIGHIRG | |
| | HTGLPGPLAQGNAYADSLTRILT | |
| MMTVB_P03365- | GRDIMKDIKVRLMTDSPDDSQDLMIGAIESNLFADQISWKSDQPVWLN | 420 |
| Pro | QWPLKQEKLQALQQLVTEQLQLGHLEESNSPWNTPVFVIKKKSGKWRL | |
| | LQDLRAVNATMHDMGALQPGLPSPVAVPKGWEIIIIDLQDCFFNIKLH | |
| | PEDCKRFAFSVPSPNFKRPYQRFQWKVLPQGMKNSPTLCQKFVDKAIL | |
| | TVRDKYQDSYIVHYMDDILLAHPSRSIVDEILTSMIQALNKHGLVVST | |
| | EKIQKYDNLKYLGTHIQGDSVSYQKLQIRTDKLRTLNDFQKLLGNINW | |
| | IRPFLKLTTGELKPLFEILNGDSNPISTRKLTPEACKALQLMNERLST | |
| | ARVKRLDLSQPWSLCILKTEYTPTACLWQDGVVEWIHLPHISPKVITP | |
| | YDIFCTQLIIKGRHRSKELFSKDPDYIVVPYTKVQFDLLLQEKEDWPI | |
| | SLLGFLGEVHFHLPKDPLLTFTLQTAIIFPHMTSTTPLEKGIVIFTDG | |
| | SANGRSVTYIQGREPIIKENTQNTAQQAEIVAVITAFEEVSQPENLYT | |
| | DSKYVTGLFPEIETATLSPRTKIYTELKHLQRLIHKRQEKFYIGHIRG | |
| | HTGLPGPLAQGNAYADSLTRILT | |
| MMTVB_P03365- | GRDIMKDIKVRLMTDSPDDSQDLMIGAIESNLFADQISWKSDQPVWLN | 421 |
| Pro_2mut | QWPLKQEKLQALQQLVTEQLQLGHLEESNSPWNTPVFVIKKKSGKWRL | |
| | LQDLRAVNATMHDMGALQPGLPSPVAVPKGWEIIIIDLQDCFFNIKLH | |
| | PEDCKRFAFSVPSPNFKRPYQRFQWKVLPQGMKNSPTLCQKFVDKAIL | |
| | TVRDKYQDSYIVHYMDDILLAHPSRSIVDEILTSMIQALNKHGLVVST | |
| | EKIQKYDNLKYLGTHIQGDSVSYQKLQIRTDKLRTLNDFQKLLGNINW | |
| | IRPFLKLTTGELKPLFEILNPDSNPISTRKLTPEACKALQLMNERLST | |
| | ARVKRLDLSQPWSLCILKTEYTPTACLWQDGVVEWIHLPHISPKVITP | |
| | YDIFCTQLIIKGRHRSKELFSKDPDYIVVPYTKVQFDLLLQEKEDWPI | |
| | SLLGFLGEVHFHLPKDPLLTFTLQTAIIFPHMTSTTPLEKGIVIFTDG | |
| | SANGRSVTYIQGREPIIKENTQNTAQQAEIVAVITAFEEVSQPFNLYT | |
| | DSKYVTGLFPEIETATLSPRTKIYTELKHLQRLIHKRQEKFYIGHIRG | |
| | HTGLPGPLAQGNAYADSLTRILT | |
| MMTVB_P03365- | GRDIMKDIKVRLMTDSPDDSQDLMIGAIESNLFADQISWKSDQPVWLN | 422 |
| Pro_2mut | QWPLKQEKLQALQQLVTEQLQLGHLEESNSPWNTPVFVIKKKSGKWRL | |
| | LQDLRAVNATMHDMGALQPGLPSPVAVPKGWEIIIIDLQDCFFNIKLH | |
| | PEDCKRFAFSVPSPNFKRPYQRFQWKVLPQGMKNSPTLCQKFVDKAIL | |
| | TVRDKYQDSYIVHYMDDILLAHPSRSIVDEILTSMIQALNKHGLVVST | |
| | EKIQKYDNLKYLGTHIQGDSVSYQKLQIRTDKLRTLNDFQKLLGNINW | |
| | IRPFLKLTTGELKPLFEILNPDSNPISTRKLTPEACKALQLMNERLST | |
| | ARVKRLDLSQPWSLCILKTEYTPTACLWQDGVVEWIHLPHISPKVITP | |
| | YDIFCTQLIIKGRHRSKELFSKDPDYIVVPYTKVQFDLLLQEKEDWPI | |
| | SLLGFLGEVHFHLPKDPLLTFTLQTAIIFPHMTSTTPLEKGIVIFTDG | |
| | SANGRSVTYIQGREPIIKENTQNTAQQAEIVAVITAFEEVSQPENLYT | |
| | DSKYVTGLFPEIETATLSPRTKIYTELKHLQRLIHKRQEKFYIGHIRG | |
| | HTGLPGPLAQGNAYADSLTRILT | |
| MMTVB_P03365- | GRDIMKDIKVRLMTDSPDDSQDLMIGAIESNLFADQISWKSDQPVWLN | 423 |
| Pro_2mutB | QWPLKQEKLQALQQLVTEQLQLGHLEESNSPWNTPVFVIKKKSGKWRL | |
| | LQDLRAVNATMHDMGALQPGLPSPVAPPKGWEIIIIDLQDCFFNIKLH | |
| | PEDCKRFAFSVPSPNFKRPYQRFQWKVLPQGMKNSPTLCQKFVDKAIL | |
| | TVRDKYQDSYIVHYMDDILLAHPSRSIVDEILTSMIQALNKHGLVVST | |
| | EKIQKYDNLKYLGTHIQGDSVSYQKLQIRTDKLRTLNDFQKLLGNINW | |
| | IRPFLKLTTGELKPLFEILNPDSNPISTRKLTPEACKALQLMNERLST | |
| | ARVKRLDLSQPWSLCILKTEYTPTACLWQDGVVEWIHLPHISPKVITP | |
| | YDIFCTQLIIKGRHRSKELFSKDPDYIVVPYTKVQFDLLLQEKEDWPI | |
| | SLLGFLGEVHFHLPKDPLLTFTLQTAIIFPHMTSTTPLEKGIVIFTDG | |
| | SANGRSVTYIQGREPIIKENTQNTAQQAEIVAVITAFEEVSQPENLYT | |
| | DSKYVTGLFPEIETATLSPRTKIYTELKHLQRLIHKRQEKFYIGHIRG | |
| | HTGLPGPLAQGNAYADSLTRILT | |
| MMTVB_P03365- | GRDIMKDIKVRLMTDSPDDSQDLMIGAIESNLFADQISWKSDQPVWLN | 424 |
| Pro_2mutB | QWPLKQEKLQALQQLVTEQLQLGHLEESNSPWNTPVFVIKKKSGKWRL | |
| | LQDLRAVNATMHDMGALQPGLPSPVAPPKGWEIIIIDLQDCFFNIKLH | |
| | PEDCKRFAFSVPSPNFKRPYQRFQWKVLPQGMKNSPTLCQKFVDKAIL | |
| | TVRDKYQDSYIVHYMDDILLAHPSRSIVDEILTSMIQALNKHGLVVST | |
| | EKIQKYDNLKYLGTHIQGDSVSYQKLQIRTDKLRTLNDFQKLLGNINW | |
| | IRPFLKLTTGELKPLFEILNPDSNPISTRKLTPEACKALQLMNERLST | |
| | ARVKRLDLSQPWSLCILKTEYTPTACLWQDGVVEWIHLPHISPKVITP | |
| | YDIFCTQLIIKGRHRSKELFSKDPDYIVVPYTKVQFDLLLQEKEDWPI | |
| | SLLGFLGEVHFHLPKDPLLTFTLQTAIIFPHMTSTTPLEKGIVIFTDG | |
| | SANGRSVTYIQGREPIIKENTQNTAQQAEIVAVITAFEEVSQPFNLYT | |
| | DSKYVTGLFPEIETATLSPRTKIYTELKHLQRLIHKRQEKFYIGHIRG | |
| | HTGLPGPLAQGNAYADSLTRILT | |
| MPMV_P07572 | LTAAIDILAPQQCAEPITWKSDEPVWVDQWPLINDKLAAAQQLVQEQL | 425 |
| | EAGHITESSSPWNTPIFVIKKKSGKWRLLQDLRAVNATMVLMGALQPG | |
| | LPSPVAIPQGYLKIIIDLKDCFFSIPLHPSDQKRFAFSLPSTNFKEPM | |
| | QRFQWKVLPQGMANSPTLCQKYVATAIHKVRHAWKQMYIIHYMDDILI | |
| | AGKDGQQVLQCFDQLKQELTAAGLHIAPEKVQLQDPYTYLGFELNGPK | |
| | ITNQKAVIRKDKLQTLNDFQKLLGDINWLRPYLKLTTGDLKPLFDTLK | |
| | GDSDPNSHRSLSKEALASLEKVETAIAEQFVTHINYSLPLIFLIENTA | |
| | LTPTGLFWQDNPIMWIHLPASPKKVLLPYYDAIADLIILGRDHSKKYF | |
| | GIEPSTIIQPYSKSQIDWLMQNTEMWPIACASEVGILDNHYPPNKLIQ | |
| | FCKLHTFVFPQIISKTPLNNALLVFTDGSSTGMAAYTLTDTTIKFQTN | |
| | LNSAQLVELQALIAVLSAFPNQPLNIYTDSAYLAHSIPLLETVAQIKH | |
| | ISETAKLFLQCQQLIYNRSIPFYIGHVRAHSGLPGPIAQGNQRADLAT | |
| | KIVASNINT | |
| MPMV_P07572_ | LTAAIDILAPQQCAEPITWKSDEPVWVDQWPLTNDKLAAAQQLVQEQL | 426 |
| 2mutB | EAGHITESSSPWNTPIFVIKKKSGKWRLLQDLRAVNATMVLMGALQPG | |
| | LPSPVAPPQGYLKIIIDLKDCFFSIPLHPSDQKRFAFSLPSTNFKEPM | |
| | QRFQWKVLPQGMANSPTLCQKYVATAIHKVRHAWKQMYIIHYMDDILI | |
| | AGKDGQQVLQCFDQLKQELTAAGLHIAPEKVQLQDPYTYLGFELNGPK | |
| | ITNQKAVIRKDKLQTLNDFQKLLGDINWLRPYLKLTTGDLKPLEDTLK | |
| | PDSDPNSHRSLSKEALASLEKVETAIAEQFVTHINYSLPLIFLIENTA | |
| | LTPTGLFWQDNPIMWIHLPASPKKVLLPYYDAIADLIILGRDHSKKYF | |
| | GIEPSTIIQPYSKSQIDWLMQNTEMWPIACASEVGILDNHYPPNKLIQ | |
| | FCKLHTFVFPQIISKTPLNNALLVFTDGSSTGMAAYTLTDTTIKFQTN | |
| | LNSAQLVELQALIAVLSAFPNQPLNIYTDSAYLAHSIPLLETVAQIKH | |
| | ISETAKLFLQCQQLIYNRSIPFYIGHVRAHSGLPGPIAQGNQRADLAT | |
| | KIVASNINT | |
| PERV_Q4VFZ2 | TLQLDDEYRLYSPLVKPDQNIQFWLEQFPQAWAETAGMGLAKQVPPQV | 427 |
| | IQLKASATPVSVRQYPLSKEAQEGIRPHVQRLIQQGILVPVQSPWNTP | |
| | LLPVRKPGTNDYRPVQDLREVNKRVQDIHPTVPNPYNLLCALPPQRSW | |
| | YTVLDLKDAFFCLRLHPTSQPLFAFEWRDPGTGRTGQLTWTRLPQGFK | |
| | NSPTIFDEALHRDLANFRIQHPQVTLLQYVDDLLLAGATKQDCLEGTK | |
| | ALLLELSDLGYRASAKKAQICRREVTYLGYSLRDGQRWLTEARKKTVV | |
| | QIPAPTTAKQVREFLGTAGFCRLWIPGFATLAAPLYPLTKEKGEFSWA | |
| | PEHQKAFDAIKKALLSAPALALPDVTKPFTLYVDERKGVARGVLTQTL | |
| | GPWRRPVAYLSKKLDPVASGWPVCLKAIAAVAILVKDADKLTLGQNIT | |
| | VIAPHALENIVRQPPDRWMTNARMTHYQSLLLTERVTFAPPAALNPAT | |
| | LLPEETDEPVTHDCHQLLIEETGVRKDLTDIPLTGEVLTWFTDGSSYV | |
| | VEGKRMAGAAVVDGTRTIWASSLPEGTSAQKAELMALTQALRLAEGKS | |
| | INIYTDSRYAFATAHVHGAIYKQRGLLTSAGREIKNKEEILSLLEALH | |
| | LPKRLAIIHCPGHQKAKDPISRGNQMADRVAKQAAQGVNLL | |
| PERV_Q4VFZ2 | TLQLDDEYRLYSPLVKPDQNIQFWLEQFPQAWAETAGMGLAKQVPPQV | 428 |
| | IQLKASATPVSVRQYPLSKEAQEGIRPHVQRLIQQGILVPVQSPWNTP | |
| | LLPVRKPGTNDYRPVQDLREVNKRVQDIHPTVPNPYNLLCALPPQRSW | |
| | YTVLDLKDAFFCLRLHPTSQPLFAFEWRDPGTGRTGQLTWTRLPQGFK | |
| | NSPTIFDEALHRDLANFRIQHPQVTLLQYVDDLLLAGATKQDCLEGTK | |
| | ALLLELSDLGYRASAKKAQICRREVTYLGYSLRDGQRWLTEARKKTVV | |
| | QIPAPTTAKQVREFLGTAGFCRLWIPGFATLAAPLYPLTKEKGEFSWA | |
| | PEHQKAFDAIKKALLSAPALALPDVTKPFTLYVDERKGVARGVLTQTL | |
| | GPWRRPVAYLSKKLDPVASGWPVCLKAIAAVAILVKDADKLTLGQNIT | |
| | VIAPHALENIVRQPPDRWMTNARMTHYQSLLLTERVTFAPPAALNPAT | |
| | LLPEETDEPVTHDCHQLLIEETGVRKDLTDIPLTGEVLTWFTDGSSYV | |
| | VEGKRMAGAAVVDGTRTIWASSLPEGTSAQKAELMALTQALRLAEGKS | |
| | INIYTDSRYAFATAHVHGAIYKQRGLLTSAGREIKNKEEILSLLEALH | |
| | LPKRLAIIHCPGHQKAKDPISRGNQMADRVAKQAAQGVNLL | |
| PERV_Q4VFZ2_ | TLQLDDEYRLYSPLVKPDQNIQFWLEQFPQAWAETAGMGLAKQVPPQV | 429 |
| 3mut | IQLKASATPVSVRQYPLSKEAQEGIRPHVQRLIQQGILVPVQSPWNTP | |
| | LLPVRKPGTNDYRPVQDLREVNKRVQDIHPTVPNPYNLLCALPPQRSW | |
| | YTVLDLKDAFFCLRLHPTSQPLFAFEWRDPGTGRTGQLTWTRLPQGFK | |
| | NSPTIFNEALHRDLANFRIQHPQVTLLQYVDDLLLAGATKQDCLEGTK | |
| | ALLLELSDLGYRASAKKAQICRREVTYLGYSLRDGQRWLTEARKKTVV | |
| | QIPAPTTAKQVREFLGTAGFCRLWIPGFATLAAPLYPLTKPKGEFSWA | |
| | PEHQKAFDAIKKALLSAPALALPDVTKPFTLYVDERKGVARGVLTQTL | |
| | GPWRRPVAYLSKKLDPVASGWPVCLKAIAAVAILVKDADKLTLGQNIT | |
| | VIAPHALENIVRQPPDRWMTNARMTHYQSLLLTERVTFAPPAALNPAT | |
| | LLPEETDEPVTHDCHQLLIEETGVRKDLTDIPLTGEVLTWFTDGSSYV | |
| | VEGKRMAGAAVVDGTRTIWASSLPEGTSAQKAELMALTQALRLAEGKS | |
| | INIYTDSRYAFATAHVHGAIYKQRGWLTSAGREIKNKEEILSLLEALH | |
| | LPKRLAIIHCPGHQKAKDPISRGNQMADRVAKQAAQGVNLL | |
| PERV_Q4VFZ2_ | TLQLDDEYRLYSPLVKPDQNIQFWLEQFPQAWAETAGMGLAKQVPPQV | 430 |
| 3mut | IQLKASATPVSVRQYPLSKEAQEGIRPHVQRLIQQGILVPVQSPWNTP | |
| | LLPVRKPGTNDYRPVQDLREVNKRVQDIHPTVPNPYNLLCALPPQRSW | |
| | YTVLDLKDAFFCLRLHPTSQPLFAFEWRDPGTGRTGQLTWTRLPQGFK | |
| | NSPTIFNEALHRDLANFRIQHPQVTLLQYVDDLLLAGATKQDCLEGTK | |
| | ALLLELSDLGYRASAKKAQICRREVTYLGYSLRDGQRWLTEARKKTVV | |
| | QIPAPTTAKQVREFLGTAGFCRLWIPGFATLAAPLYPLTKPKGEFSWA | |
| | PEHQKAFDAIKKALLSAPALALPDVTKPFTLYVDERKGVARGVLTQTL | |
| | GPWRRPVAYLSKKLDPVASGWPVCLKAIAAVAILVKDADKLTLGQNIT | |
| | VIAPHALENIVRQPPDRWMTNARMTHYQSLLLTERVTFAPPAALNPAT | |
| | LLPEETDEPVTHDCHQLLIEETGVRKDLTDIPLTGEVLTWFTDGSSYV | |
| | VEGKRMAGAAVVDGTRTIWASSLPEGTSAQKAELMALTQALRLAEGKS | |
| | INIYTDSRYAFATAHVHGAIYKQRGWLTSAGREIKNKEEILSLLEALH | |
| | LPKRLAIIHCPGHQKAKDPISRGNQMADRVAKQAAQGVNLL | |
| PERV_Q4VFZ2_ | LDDEYRLYSPLVKPDQNIQFWLEQFPQAWAETAGMGLAKQVPPQVIQL | 431 |
| 3mutA_WS | KASATPVSVRQYPLSKEAQEGIRPHVQRLIQQGILVPVQSPWNTPLLP | |
| | VRKPGTNDYRPVQDLREVNKRVQDIHPTVPNPYNLLCALPPQRSWYTV | |
| | LDLKDAFFCLRLHPTSQPLFAFEWRDPGTGRTGQLTWTRLPQGFKNSP | |
| | TIFNEALHRDLANFRIQHPQVTLLQYVDDLLLAGATKQDCLEGTKALL | |
| | LELSDLGYRASAKKAQICRREVTYLGYSLRDGQRWLTEARKKTVVQIP | |
| | APTTAKQVREFLGKAGFCRLFIPGFATLAAPLYPLTKPKGEFSWAPEH | |
| | QKAFDAIKKALLSAPALALPDVTKPFTLYVDERKGVARGVLTQTLGPW | |
| | RRPVAYLSKKLDPVASGWPVCLKAIAAVAILVKDADKLTLGQNITVIA | |
| | PHALENIVRQPPDRWMTNARMTHYQSLLLTERVTFAPPAALNPATLLP | |
| | EETDEPVTHDCHQLLIEETGVRKDLTDIPLTGEVLTWFTDGSSYVVEG | |
| | KRMAGAAVVDGTRTIWASSLPEGTSAQKAELMALTQALRLAEGKSINI | |
| | YTDSRYAFATAHVHGAIYKQRGWLTSAGREIKNKEEILSLLEALHLPK | |
| | RLAIIHCPGHQKAKDPISRGNQMADRVAKQAAQGVNLLP | |
| PERV_Q4VFZ2_ | LDDEYRLYSPLVKPDQNIQFWLEQFPQAWAETAGMGLAKQVPPQVIQL | 432 |
| 3mutA_WS | KASATPVSVRQYPLSKEAQEGIRPHVQRLIQQGILVPVQSPWNTPLLP | |
| | VRKPGTNDYRPVQDLREVNKRVQDIHPTVPNPYNLLCALPPQRSWYTV | |
| | LDLKDAFFCLRLHPTSQPLFAFEWRDPGTGRTGQLTWTRLPQGFKNSP | |
| | TIFNEALHRDLANFRIQHPQVTLLQYVDDLLLAGATKQDCLEGTKALL | |
| | LELSDLGYRASAKKAQICRREVTYLGYSLRDGQRWLTEARKKTVVQIP | |
| | APTTAKQVREFLGKAGFCRLFIPGFATLAAPLYPLTKPKGEFSWAPEH | |
| | QKAFDAIKKALLSAPALALPDVTKPFTLYVDERKGVARGVLTQTLGPW | |
| | RRPVAYLSKKLDPVASGWPVCLKAIAAVAILVKDADKLTLGQNITVIA | |
| | PHALENIVRQPPDRWMTNARMTHYQSLLLTERVTFAPPAALNPATLLP | |
| | EETDEPVTHDCHQLLIEETGVRKDLTDIPLTGEVLTWFTDGSSYVVEG | |
| | KRMAGAAVVDGTRTIWASSLPEGTSAQKAELMALTQALRLAEGKSINI | |
| | YTDSRYAFATAHVHGAIYKQRGWLTSAGREIKNKEEILSLLEALHLPK | |
| | RLAIIHCPGHQKAKDPISRGNQMADRVAKQAAQGVNLLP | |
| SFV1_P23074 | MDPLQLLQPLEAEIKGTKLKAHWNSGATITCVPEAFLEDERPIQTMLI | 433 |
| | KTIHGEKQQDVYYLTFKVQGRKVEAEVLASPYDYILLNPSDVPWLMKK | |
| | PLQLTVLVPLHEYQERLLQQTALPKEQKELLQKLFLKYDALWQHWENQ | |
| | VGHRRIKPHNIATGTLAPRPQKQYPINPKAKPSIQIVIDDLLKQGVLI | |
| | QQNSTMNTPVYPVPKPDGKWRMVLDYREVNKTIPLIAAQNQHSAGILS | |
| | SIYRGKYKTTLDLINGFWAHPITPESYWLTAFTWQGKQYCWTRLPQGF | |
| | LNSPALFTADVVDLLKEIPNVQAYVDDIYISHDDPQEHLEQLEKIFSI | |
| | LLNAGYVVSLKKSEIAQREVEFLGFNITKEGRGLTDTFKQKLLNITPP | |
| | KDLKQLQSILGLLNFARNFIPNYSELVKPLYTIVANANGKFISWTEDN | |
| | SNQLQHIISVLNQADNLEERNPETRLIIKVNSSPSAGYIRYYNEGSKR | |
| | PIMYVNYIFSKAEAKFTQTEKLLTTMHKGLIKAMDLAMGQEILVYSPI | |
| | VSMTKIQRTPLPERKALPVRWITWMTYLEDPRIQFHYDKSLPELQQIP | |
| | NVTEDVIAKTKHPSEFAMVFYTDGSAIKHPDVNKSHSAGMGIAQVQFI | |
| | PEYKIVHQWSIPLGDHTAQLAEIAAVEFACKKALKISGPVLIVTDSFY | |
| | VAESANKELPYWKSNGFLNNKKKPLRHVSKWKSIAECLQLKPDIIIMH | |
| | EKGHQQPMTTLHTEGNNLADKLATQGSYVVH | |
| SFV1_P23074_ | MDPLQLLQPLEAEIKGTKLKAHWNSGATITCVPEAFLEDERPIQTMLI | 434 |
| 2mut | KTIHGEKQQDVYYLTFKVQGRKVEAEVLASPYDYILLNPSDVPWLMKK | |
| | PLQLTVLVPLHEYQERLLQQTALPKEQKELLQKLFLKYDALWQHWENQ | |
| | VGHRRIKPHNIATGTLAPRPQKQYPINPKAKPSIQIVIDDLLKQGVLI | |
| | QQNSTMNTPVYPVPKPDGKWRMVLDYREVNKTIPLIAAQNQHSAGILS | |
| | SIYRGKYKTTLDLINGFWAHPITPESYWLTAFTWQGKQYCWTRLPQGF | |
| | LNSPALFNADVVDLLKEIPNVQAYVDDIYISHDDPQEHLEQLEKIFSI | |
| | LLNAGYVVSLKKSEIAQREVEFLGFNITKEGRGLTDTFKQKLLNITPP | |
| | KDLKQLQSILGLLNFARNFIPNYSELVKPLYTIVAPANGKFISWTEDN | |
| | SNQLQHIISVLNQADNLEERNPETRLIIKVNSSPSAGYIRYYNEGSKR | |
| | PIMYVNYIFSKAEAKFTQTEKLLTTMHKGLIKAMDLAMGQEILVYSPI | |
| | VSMTKIQRTPLPERKALPVRWITWMTYLEDPRIQFHYDKSLPELQQIP | |
| | NVTEDVIAKTKHPSEFAMVFYTDGSAIKHPDVNKSHSAGMGIAQVQFI | |
| | PEYKIVHQWSIPLGDHTAQLAEIAAVEFACKKALKISGPVLIVTDSFY | |
| | VAESANKELPYWKSNGFLNNKKKPLRHVSKWKSIAECLQLKPDIIIMH | |
| | EKGHQQPMTTLHTEGNNLADKLATQGSYVVH | |
| SFV1_P23074_ | MDPLQLLQPLEAEIKGTKLKAHWNSGATITCVPEAFLEDERPIQTMLI | 435 |
| 2mutA | KTIHGEKQQDVYYLTFKVQGRKVEAEVLASPYDYILLNPSDVPWLMKK | |
| | PLQLTVLVPLHEYQERLLQQTALPKEQKELLQKLFLKYDALWQHWENQ | |
| | VGHRRIKPHNIATGTLAPRPQKQYPINPKAKPSIQIVIDDLLKQGVLI | |
| | QQNSTMNTPVYPVPKPDGKWRMVLDYREVNKTIPLIAAQNQHSAGILS | |
| | SIYRGKYKTTLDLINGFWAHPITPESYWLTAFTWQGKQYCWTRLPQGF | |
| | LNSPALFNADVVDLLKEIPNVQAYVDDIYISHDDPQEHLEQLEKIFSI | |
| | LLNAGYVVSLKKSEIAQREVEFLGFNITKEGRGLTDTFKQKLLNITPP | |
| | KDLKQLQSILGKLNFARNFIPNYSELVKPLYTIVAPANGKFISWTEDN | |
| | SNQLQHIISVLNQADNLEERNPETRLIIKVNSSPSAGYIRYYNEGSKR | |
| | PIMYVNYIFSKAEAKFTQTEKLLTTMHKGLIKAMDLAMGQEILVYSPI | |
| | VSMTKIQRTPLPERKALPVRWITWMTYLEDPRIQFHYDKSLPELQQIP | |
| | NVTEDVIAKTKHPSEFAMVFYTDGSAIKHPDVNKSHSAGMGIAQVQFI | |
| | PEYKIVHQWSIPLGDHTAQLAEIAAVEFACKKALKISGPVLIVTDSFY | |
| | VAESANKELPYWKSNGFLNNKKKPLRHVSKWKSIAECLQLKPDIIIMH | |
| | EKGHQQPMTTLHTEGNNLADKLATQGSYVVH | |
| SFV1_P23074- | VPWLMKKPLQLTVLVPLHEYQERLLQQTALPKEQKELLQKLFLKYDAL | 436 |
| Pro | WQHWENQVGHRRIKPHNIATGTLAPRPQKQYPINPKAKPSIQIVIDDL | |
| | LKQGVLIQQNSTMNTPVYPVPKPDGKWRMVLDYREVNKTIPLIAAQNQ | |
| | HSAGILSSIYRGKYKTTLDLINGFWAHPITPESYWLTAFTWQGKQYCW | |
| | TRLPQGFLNSPALFTADVVDLLKEIPNVQAYVDDIYISHDDPQEHLEQ | |
| | LEKIFSILLNAGYVVSLKKSEIAQREVEFLGFNITKEGRGLTDTFKQK | |
| | LLNITPPKDLKQLQSILGLLNFARNFIPNYSELVKPLYTIVANANGKF | |
| | ISWTEDNSNQLQHIISVLNQADNLEERNPETRLIIKVNSSPSAGYIRY | |
| | YNEGSKRPIMYVNYIFSKAEAKFTQTEKLLTTMHKGLIKAMDLAMGQE | |
| | ILVYSPIVSMTKIQRTPLPERKALPVRWITWMTYLEDPRIQFHYDKSL | |
| | PELQQIPNVTEDVIAKTKHPSEFAMVFYTDGSAIKHPDVNKSHSAGMG | |
| | IAQVQFIPEYKIVHQWSIPLGDHTAQLAEIAAVEFACKKALKISGPVL | |
| | IVTDSFYVAESANKELPYWKSNGFLNNKKKPLRHVSKWKSIAECLQLK | |
| | PDIIIMHEKGHQQPMTTLHTEGNNLADKLATQGSYVVH | |
| SFV1_P23074- | VPWLMKKPLQLTVLVPLHEYQERLLQQTALPKEQKELLQKLFLKYDAL | 437 |
| Pro_2mut | WQHWENQVGHRRIKPHNIATGTLAPRPQKQYPINPKAKPSIQIVIDDL | |
| | LKQGVLIQQNSTMNTPVYPVPKPDGKWRMVLDYREVNKTIPLIAAQNQ | |
| | HSAGILSSIYRGKYKTTLDLINGFWAHPITPESYWLTAFTWQGKQYCW | |
| | TRLPQGFLNSPALFNADVVDLLKEIPNVQAYVDDIYISHDDPQEHLEQ | |
| | LEKIFSILLNAGYVVSLKKSEIAQREVEFLGFNITKEGRGLTDTFKQK | |
| | LLNITPPKDLKQLQSILGLLNFARNFIPNYSELVKPLYTIVAPANGKF | |
| | ISWTEDNSNQLQHIISVLNQADNLEERNPETRLIIKVNSSPSAGYIRY | |
| | YNEGSKRPIMYVNYIFSKAEAKFTQTEKLLTTMHKGLIKAMDLAMGQE | |
| | ILVYSPIVSMTKIQRTPLPERKALPVRWITWMTYLEDPRIQFHYDKSL | |
| | PELQQIPNVTEDVIAKTKHPSEFAMVFYTDGSAIKHPDVNKSHSAGMG | |
| | IAQVQFIPEYKIVHQWSIPLGDHTAQLAEIAAVEFACKKALKISGPVL | |
| | IVTDSFYVAESANKELPYWKSNGFLNNKKKPLRHVSKWKSIAECLQLK | |
| | PDIIIMHEKGHQQPMTTLHTEGNNLADKLATQGSYVVH | |
| SFV1_P23074- | VPWLMKKPLQLTVLVPLHEYQERLLQQTALPKEQKELLQKLFLKYDAL | 438 |
| Pro_2mutA | WQHWENQVGHRRIKPHNIATGTLAPRPQKQYPINPKAKPSIQIVIDDL | |
| | LKQGVLIQQNSTMNTPVYPVPKPDGKWRMVLDYREVNKTIPLIAAQNQ | |
| | HSAGILSSIYRGKYKTTLDLINGFWAHPITPESYWLTAFTWQGKQYCW | |
| | TRLPQGELNSPALFNADVVDLLKEIPNVQAYVDDIYISHDDPQEHLEQ | |
| | LEKIFSILLNAGYVVSLKKSEIAQREVEFLGENITKEGRGLTDTFKQK | |
| | LLNITPPKDLKQLQSILGKLNFARNFIPNYSELVKPLYTIVAPANGKF | |
| | ISWTEDNSNQLQHIISVLNQADNLEERNPETRLIIKVNSSPSAGYIRY | |
| | YNEGSKRPIMYVNYIFSKAEAKFTQTEKLLTTMHKGLIKAMDLAMGQE | |
| | ILVYSPIVSMTKIQRTPLPERKALPVRWITWMTYLEDPRIQFHYDKSL | |
| | PELQQIPNVTEDVIAKTKHPSEFAMVFYTDGSAIKHPDVNKSHSAGMG | |
| | IAQVQFIPEYKIVHQWSIPLGDHTAQLAEIAAVEFACKKALKISGPVL | |
| | IVTDSFYVAESANKELPYWKSNGFLNNKKKPLRHVSKWKSIAECLQLK | |
| | PDIIIMHEKGHQQPMTTLHTEGNNLADKLATQGSYVVH | |
| SFV3L_P27401 | MDPLQLLQPLEAEIKGTKLKAHWNSGATITCVPQAFLEEEVPIKNIWI | 439 |
| | KTIHGEKEQPVYYLTFKIQGRKVEAEVISSPYDYILVSPSDIPWLMKK | |
| | PLQLTTLVPLQEYEERLLKQTMLTGSYKEKLQSLFLKYDALWQHWENQ | |
| | VGHRRIKPHHIATGTVNPRPQKQYPINPKAKASIQTVINDLLKQGVLI | |
| | QQNSIMNTPVYPVPKPDGKWRMVLDYREVNKTIPLIAAQNQHSAGILS | |
| | SIFRGKYKTTLDLSNGFWAHSITPESYWLTAFTWLGQQYCWTRLPQGE | |
| | LNSPALFTADVVDLLKEVPNVQVYVDDIYISHDDPREHLEQLEKVESL | |
| | LLNAGYVVSLKKSEIAQHEVEFLGFNITKEGRGLTETFKQKLLNITPP | |
| | RDLKQLQSILGLLNFARNFIPNFSELVKPLYNIIATANGKYITWTTDN | |
| | SQQLQNIISMLNSAENLEERNPEVRLIMKVNTSPSAGYIRFYNEFAKR | |
| | PIMYLNYVYTKAEVKFTNTEKLLTTIHKGLIKALDLGMGQEILVYSPI | |
| | VSMTKIQKTPLPERKALPIRWITWMSYLEDPRIQFHYDKTLPELQQVP | |
| | TVTDDIIAKIKHPSEFSMVFYTDGSAIKHPNVNKSHNAGMGIAQVQFK | |
| | PEFTVINTWSIPLGDHTAQLAEVAAVEFACKKALKIDGPVLIVTDSFY | |
| | VAESVNKELPYWQSNGFFNNKKKPLKHVSKWKSIADCIQLKPDIIIIH | |
| | EKGHQPTASTFHTEGNNLADKLATQGSYVVN | |
| SFV3L_P27401_ | MDPLQLLQPLEAEIKGTKLKAHWNSGATITCVPQAFLEEEVPIKNIWI | 440 |
| 2mut | KTIHGEKEQPVYYLTFKIQGRKVEAEVISSPYDYILVSPSDIPWLMKK | |
| | PLQLTTLVPLQEYEERLLKQTMLTGSYKEKLQSLFLKYDALWQHWENQ | |
| | VGHRRIKPHHIATGTVNPRPQKQYPINPKAKASIQTVINDLLKQGVLI | |
| | QQNSIMNTPVYPVPKPDGKWRMVLDYREVNKTIPLIAAQNQHSAGILS | |
| | SIFRGKYKTTLDLSNGFWAHSITPESYWLTAFTWLGQQYCWTRLPQGF | |
| | LNSPALFNADVVDLLKEVPNVQVYVDDIYISHDDPREHLEQLEKVESL | |
| | LLNAGYVVSLKKSEIAQHEVEFLGFNITKEGRGLTETFKQKLLNITPP | |
| | RDLKQLQSILGLLNFARNFIPNFSELVKPLYNIIATAPGKYITWTTDN | |
| | SQQLQNIISMLNSAENLEERNPEVRLIMKVNTSPSAGYIRFYNEFAKR | |
| | PIMYLNYVYTKAEVKFTNTEKLLTTIHKGLIKALDLGMGQEILVYSPI | |
| | VSMTKIQKTPLPERKALPIRWITWMSYLEDPRIQFHYDKTLPELQQVP | |
| | TVTDDIIAKIKHPSEFSMVFYTDGSAIKHPNVNKSHNAGMGIAQVQFK | |
| | PEFTVINTWSIPLGDHTAQLAEVAAVEFACKKALKIDGPVLIVTDSFY | |
| | VAESVNKELPYWQSNGFFNNKKKPLKHVSKWKSIADCIQLKPDIIIIH | |
| | EKGHQPTASTFHTEGNNLADKLATQGSYVVN | |
| SFV3L_P27401_ | MDPLQLLQPLEAEIKGTKLKAHWNSGATITCVPQAFLEEEVPIKNIWI | 441 |
| 2mutA | KTIHGEKEQPVYYLTFKIQGRKVEAEVISSPYDYILVSPSDIPWLMKK | |
| | PLQLTTLVPLQEYEERLLKQTMLTGSYKEKLQSLFLKYDALWQHWENQ | |
| | VGHRRIKPHHIATGTVNPRPQKQYPINPKAKASIQTVINDLLKQGVLI | |
| | QQNSIMNTPVYPVPKPDGKWRMVLDYREVNKTIPLIAAQNQHSAGILS | |
| | SIFRGKYKTTLDLSNGFWAHSITPESYWLTAFTWLGQQYCWTRLPQGF | |
| | LNSPALFNADVVDLLKEVPNVQVYVDDIYISHDDPREHLEQLEKVESL | |
| | LLNAGYVVSLKKSEIAQHEVEFLGFNITKEGRGLTETFKQKLLNITPP | |
| | RDLKQLQSILGKLNFARNFIPNFSELVKPLYNIIATAPGKYITWTTDN | |
| | SQQLQNIISMLNSAENLEERNPEVRLIMKVNTSPSAGYIRFYNEFAKR | |
| | PIMYLNYVYTKAEVKFTNTEKLLTTIHKGLIKALDLGMGQEILVYSPI | |
| | VSMTKIQKTPLPERKALPIRWITWMSYLEDPRIQFHYDKTLPELQQVP | |
| | TVTDDIIAKIKHPSEFSMVFYTDGSAIKHPNVNKSHNAGMGIAQVQFK | |
| | PEFTVINTWSIPLGDHTAQLAEVAAVEFACKKALKIDGPVLIVTDSFY | |
| | VAESVNKELPYWQSNGFFNNKKKPLKHVSKWKSIADCIQLKPDIIIIH | |
| | EKGHQPTASTFHTEGNNLADKLATQGSYVVN | |
| SFV3L_P27401- | IPWLMKKPLQLTTLVPLQEYEERLLKQTMLTGSYKEKLQSLFLKYDAL | 442 |
| Pro | WQHWENQVGHRRIKPHHIATGTVNPRPQKQYPINPKAKASIQTVINDL | |
| | LKQGVLIQQNSIMNTPVYPVPKPDGKWRMVLDYREVNKTIPLIAAQNQ | |
| | HSAGILSSIFRGKYKTTLDLSNGFWAHSITPESYWLTAFTWLGQQYCW | |
| | TRLPQGFLNSPALFTADVVDLLKEVPNVQVYVDDIYISHDDPREHLEQ | |
| | LEKVFSLLLNAGYVVSLKKSEIAQHEVEFLGFNITKEGRGLTETFKQK | |
| | LLNITPPRDLKQLQSILGLLNFARNFIPNFSELVKPLYNIIATANGKY | |
| | ITWTTDNSQQLQNIISMLNSAENLEERNPEVRLIMKVNTSPSAGYIRF | |
| | YNEFAKRPIMYLNYVYTKAEVKFTNTEKLLTTIHKGLIKALDLGMGQE | |
| | ILVYSPIVSMTKIQKTPLPERKALPIRWITWMSYLEDPRIQFHYDKTL | |
| | PELQQVPTVTDDIIAKIKHPSEFSMVFYTDGSAIKHPNVNKSHNAGMG | |
| | IAQVQFKPEFTVINTWSIPLGDHTAQLAEVAAVEFACKKALKIDGPVL | |
| | IVTDSFYVAESVNKELPYWQSNGFFNNKKKPLKHVSKWKSIADCIQLK | |
| | PDIIIIHEKGHQPTASTFHTEGNNLADKLATQGSYVVN | |
| SFV3L_P27401- | IPWLMKKPLQLTTLVPLQEYEERLLKQTMLTGSYKEKLQSLFLKYDAL | 443 |
| Pro_2mut | WQHWENQVGHRRIKPHHIATGTVNPRPQKQYPINPKAKASIQTVINDL | |
| | LKQGVLIQQNSIMNTPVYPVPKPDGKWRMVLDYREVNKTIPLIAAQNQ | |
| | HSAGILSSIFRGKYKTTLDLSNGFWAHSITPESYWLTAFTWLGQQYCW | |
| | TRLPQGFLNSPALFNADVVDLLKEVPNVQVYVDDIYISHDDPREHLEQ | |
| | LEKVFSLLLNAGYVVSLKKSEIAQHEVEFLGFNITKEGRGLTETFKQK | |
| | LLNITPPRDLKQLQSILGLLNFARNFIPNFSELVKPLYNIIATAPGKY | |
| | ITWTTDNSQQLQNIISMLNSAENLEERNPEVRLIMKVNTSPSAGYIRF | |
| | YNEFAKRPIMYLNYVYTKAEVKFTNTEKLLTTIHKGLIKALDLGMGQE | |
| | ILVYSPIVSMTKIQKTPLPERKALPIRWITWMSYLEDPRIQFHYDKTL | |
| | PELQQVPTVTDDIIAKIKHPSEFSMVFYTDGSAIKHPNVNKSHNAGMG | |
| | IAQVQFKPEFTVINTWSIPLGDHTAQLAEVAAVEFACKKALKIDGPVL | |
| | IVTDSFYVAESVNKELPYWQSNGFFNNKKKPLKHVSKWKSIADCIQLK | |
| | PDIIIIHEKGHQPTASTFHTEGNNLADKLATQGSYVVN | |
| SFV3L_P27401- | IPWLMKKPLQLTTLVPLQEYEERLLKQTMLTGSYKEKLQSLFLKYDAL | 444 |
| Pro_2mutA | WQHWENQVGHRRIKPHHIATGTVNPRPQKQYPINPKAKASIQTVINDL | |
| | LKQGVLIQQNSIMNTPVYPVPKPDGKWRMVLDYREVNKTIPLIAAQNQ | |
| | HSAGILSSIFRGKYKTTLDLSNGFWAHSITPESYWLTAFTWLGQQYCW | |
| | TRLPQGFLNSPALFNADVVDLLKEVPNVQVYVDDIYISHDDPREHLEQ | |
| | LEKVFSLLLNAGYVVSLKKSEIAQHEVEFLGFNITKEGRGLTETFKQK | |
| | LLNITPPRDLKQLQSILGKLNFARNFIPNFSELVKPLYNIIATAPGKY | |
| | ITWTTDNSQQLQNIISMLNSAENLEERNPEVRLIMKVNTSPSAGYIRF | |
| | YNEFAKRPIMYLNYVYTKAEVKFTNTEKLLTTIHKGLIKALDLGMGQE | |
| | ILVYSPIVSMTKIQKTPLPERKALPIRWITWMSYLEDPRIQFHYDKTL | |
| | PELQQVPTVTDDIIAKIKHPSEFSMVFYTDGSAIKHPNVNKSHNAGMG | |
| | IAQVQFKPEFTVINTWSIPLGDHTAQLAEVAAVEFACKKALKIDGPVL | |
| | IVTDSFYVAESVNKELPYWQSNGFFNNKKKPLKHVSKWKSIADCIQLK | |
| | PDIIIIHEKGHQPTASTFHTEGNNLADKLATQGSYVVN | |
| SFVCP_Q87040 | MNPLQLLQPLPAEVKGTKLLAHWNSGATITCIPESFLEDEQPIKQTLI | 445 |
| | KTIHGEKQQNVYYLTFKVKGRKVEAEVIASPYEYILLSPTDVPWLTQQ | |
| | PLQLTILVPLQEYQDRILNKTALPEEQKQQLKALFTKYDNLWQHWENQ | |
| | VGHRKIRPHNIATGDYPPRPQKQYPINPKAKPSIQIVIDDLLKQGVLT | |
| | PQNSTMNTPVYPVPKPDGRWRMVLDYREVNKTIPLTAAQNQHSAGILA | |
| | TIVRQKYKTTLDLANGFWAHPITPDSYWLTAFTWQGKQYCWTRLPQGF | |
| | LNSPALFTADAVDLLKEVPNVQVYVDDIYLSHDNPHEHIQQLEKVFQI | |
| | LLQAGYVVSLKKSEIGQRTVEFLGFNITKEGRGLTDTFKTKLLNVTPP | |
| | KDLKQLQSILGLLNFARNFIPNFAELVQTLYNLIASSKGKYIEWTEDN | |
| | TKQLNKVIEALNTASNLEERLPDQRLVIKVNTSPSAGYVRYYNESGKK | |
| | PIMYLNYVFSKAELKFSMLEKLLTTMHKALIKAMDLAMGQEILVYSPI | |
| | VSMTKIQKTPLPERKALPIRWITWMTYLEDPRIQFHYDKTLPELKHIP | |
| | DVYTSSIPPLKHPSQYEGVFCTDGSAIKSPDPTKSNNAGMGIVHAIYN | |
| | PEYKILNQWSIPLGHHTAQMAEIAAVEFACKKALKVPGPVLVITDSFY | |
| | VAESANKELPYWKSNGFVNNKKEPLKHISKWKSIAECLSIKPDITIQH | |
| | EKGHQPINTSIHTEGNALADKLATQGSYVVN | |
| SFVCP_Q87040_ | MNPLQLLQPLPAEVKGTKLLAHWNSGATITCIPESFLEDEQPIKQTLI | 446 |
| 2mut | KTIHGEKQQNVYYLTFKVKGRKVEAEVIASPYEYILLSPTDVPWLTQQ | |
| | PLQLTILVPLQEYQDRILNKTALPEEQKQQLKALFTKYDNLWQHWENQ | |
| | VGHRKIRPHNIATGDYPPRPQKQYPINPKAKPSIQIVIDDLLKQGVLT | |
| | PQNSTMNTPVYPVPKPDGRWRMVLDYREVNKTIPLTAAQNQHSAGILA | |
| | TIVRQKYKTTLDLANGFWAHPITPDSYWLTAFTWQGKQYCWTRLPQGF | |
| | LNSPALFNADAVDLLKEVPNVQVYVDDIYLSHDNPHEHIQQLEKVFQI | |
| | LLQAGYVVSLKKSEIGQRTVEFLGFNITKEGRGLTDTFKTKLLNVTPP | |
| | KDLKQLQSILGLLNFARNFIPNFAELVQTLYNLIASSPGKYIEWTEDN | |
| | TKQLNKVIEALNTASNLEERLPDQRLVIKVNTSPSAGYVRYYNESGKK | |
| | PIMYLNYVFSKAELKFSMLEKLLTTMHKALIKAMDLAMGQEILVYSPI | |
| | VSMTKIQKTPLPERKALPIRWITWMTYLEDPRIQFHYDKTLPELKHIP | |
| | DVYTSSIPPLKHPSQYEGVFCTDGSAIKSPDPTKSNNAGMGIVHAIYN | |
| | PEYKILNQWSIPLGHHTAQMAEIAAVEFACKKALKVPGPVLVITDSFY | |
| | VAESANKELPYWKSNGFVNNKKEPLKHISKWKSIAECLSIKPDITIQH | |
| | EKGHQPINTSIHTEGNALADKLATQGSYVVN | |
| SFVCP_Q87040_ | MNPLQLLQPLPAEVKGTKLLAHWNSGATITCIPESFLEDEQPIKQTLI | 447 |
| 2mutA | KTIHGEKQQNVYYLTFKVKGRKVEAEVIASPYEYILLSPTDVPWLTQQ | |
| | PLQLTILVPLQEYQDRILNKTALPEEQKQQLKALFTKYDNLWQHWENQ | |
| | VGHRKIRPHNIATGDYPPRPQKQYPINPKAKPSIQIVIDDLLKQGVLT | |
| | PQNSTMNTPVYPVPKPDGRWRMVLDYREVNKTIPLTAAQNQHSAGILA | |
| | TIVRQKYKTTLDLANGFWAHPITPDSYWLTAFTWQGKQYCWTRLPQGF | |
| | LNSPALFNADAVDLLKEVPNVQVYVDDIYLSHDNPHEHIQQLEKVFQI | |
| | LLQAGYVVSLKKSEIGQRTVEFLGFNITKEGRGLTDTFKTKLLNVTPP | |
| | KDLKQLQSILGKLNFARNFIPNFAELVQTLYNLIASSPGKYIEWTEDN | |
| | TKQLNKVIEALNTASNLEERLPDQRLVIKVNTSPSAGYVRYYNESGKK | |
| | PIMYLNYVFSKAELKFSMLEKLLTTMHKALIKAMDLAMGQEILVYSPI | |
| | VSMTKIQKTPLPERKALPIRWITWMTYLEDPRIQFHYDKTLPELKHIP | |
| | DVYTSSIPPLKHPSQYEGVFCTDGSAIKSPDPTKSNNAGMGIVHAIYN | |
| | PEYKILNQWSIPLGHHTAQMAEIAAVEFACKKALKVPGPVLVITDSFY | |
| | VAESANKELPYWKSNGFVNNKKEPLKHISKWKSIAECLSIKPDITIQH | |
| | EKGHQPINTSIHTEGNALADKLATQGSYVVN | |
| SFVCP_Q87040- | VPWLTQQPLQLTILVPLQEYQDRILNKTALPEEQKQQLKALFTKYDNL | 448 |
| Pro | WQHWENQVGHRKIRPHNIATGDYPPRPQKQYPINPKAKPSIQIVIDDL | |
| | LKQGVLTPQNSTMNTPVYPVPKPDGRWRMVLDYREVNKTIPLTAAQNQ | |
| | HSAGILATIVRQKYKTTLDLANGFWAHPITPDSYWLTAFTWQGKQYCW | |
| | TRLPQGFLNSPALFTADAVDLLKEVPNVQVYVDDIYLSHDNPHEHIQQ | |
| | LEKVFQILLQAGYVVSLKKSEIGQRTVEFLGFNITKEGRGLTDTFKTK | |
| | LLNVTPPKDLKQLQSILGLLNFARNFIPNFAELVQTLYNLIASSKGKY | |
| | IEWTEDNTKQLNKVIEALNTASNLEERLPDQRLVIKVNTSPSAGYVRY | |
| | YNESGKKPIMYLNYVFSKAELKFSMLEKLLTTMHKALIKAMDLAMGQE | |
| | ILVYSPIVSMTKIQKTPLPERKALPIRWITWMTYLEDPRIQFHYDKTL | |
| | PELKHIPDVYTSSIPPLKHPSQYEGVFCTDGSAIKSPDPTKSNNAGMG | |
| | IVHAIYNPEYKILNQWSIPLGHHTAQMAEIAAVEFACKKALKVPGPVL | |
| | VITDSFYVAESANKELPYWKSNGFVNNKKEPLKHISKWKSIAECLSIK | |
| | PDITIQHEKGHQPINTSIHTEGNALADKLATQGSYVVN | |
| SFVCP_Q87040- | VPWLTQQPLQLTILVPLQEYQDRILNKTALPEEQKQQLKALFTKYDNL | 449 |
| Pro_2mut | WQHWENQVGHRKIRPHNIATGDYPPRPQKQYPINPKAKPSIQIVIDDL | |
| | LKQGVLTPQNSTMNTPVYPVPKPDGRWRMVLDYREVNKTIPLTAAQNQ | |
| | HSAGILATIVRQKYKTTLDLANGFWAHPITPDSYWLTAFTWQGKQYCW | |
| | TRLPQGFLNSPALFNADAVDLLKEVPNVQVYVDDIYLSHDNPHEHIQQ | |
| | LEKVFQILLQAGYVVSLKKSEIGQRTVEFLGFNITKEGRGLTDTFKTK | |
| | LLNVTPPKDLKQLQSILGLLNFARNFIPNFAELVQTLYNLIASSPGKY | |
| | IEWTEDNTKQLNKVIEALNTASNLEERLPDQRLVIKVNTSPSAGYVRY | |
| | YNESGKKPIMYLNYVFSKAELKFSMLEKLLTTMHKALIKAMDLAMGQE | |
| | ILVYSPIVSMTKIQKTPLPERKALPIRWITWMTYLEDPRIQFHYDKTL | |
| | PELKHIPDVYTSSIPPLKHPSQYEGVFCTDGSAIKSPDPTKSNNAGMG | |
| | IVHAIYNPEYKILNQWSIPLGHHTAQMAEIAAVEFACKKALKVPGPVL | |
| | VITDSFYVAESANKELPYWKSNGFVNNKKEPLKHISKWKSIAECLSIK | |
| | PDITIQHEKGHQPINTSIHTEGNALADKLATQGSYVVN | |
| SFVCP_Q87040- | VPWLTQQPLQLTILVPLQEYQDRILNKTALPEEQKQQLKALFTKYDNL | 450 |
| Pro_2mutA | WQHWENQVGHRKIRPHNIATGDYPPRPQKQYPINPKAKPSIQIVIDDL | |
| | LKQGVLTPQNSTMNTPVYPVPKPDGRWRMVLDYREVNKTIPLTAAQNQ | |
| | HSAGILATIVRQKYKTTLDLANGFWAHPITPDSYWLTAFTWQGKQYCW | |
| | TRLPQGFLNSPALFNADAVDLLKEVPNVQVYVDDIYLSHDNPHEHIQQ | |
| | LEKVFQILLQAGYVVSLKKSEIGQRTVEFLGFNITKEGRGLTDTFKTK | |
| | LLNVTPPKDLKQLQSILGKLNFARNFIPNFAELVQTLYNLIASSPGKY | |
| | IEWTEDNTKQLNKVIEALNTASNLEERLPDQRLVIKVNTSPSAGYVRY | |
| | YNESGKKPIMYLNYVFSKAELKFSMLEKLLTTMHKALIKAMDLAMGQE | |
| | ILVYSPIVSMTKIQKTPLPERKALPIRWITWMTYLEDPRIQFHYDKTL | |
| | PELKHIPDVYTSSIPPLKHPSQYEGVFCTDGSAIKSPDPTKSNNAGMG | |
| | IVHAIYNPEYKILNQWSIPLGHHTAQMAEIAAVEFACKKALKVPGPVL | |
| | VITDSFYVAESANKELPYWKSNGFVNNKKEPLKHISKWKSIAECLSIK | |
| | PDITIQHEKGHQPINTSIHTEGNALADKLATQGSYVVN | |
| SMRVH_P03364 | PRSRAIDIPVPHADKISWKITDPVWVDQWPLTYEKTLAAIALVQEQLA | 451 |
| | AGHIEPTNSPWNTPIFIIKKKSGSWRLLQDLRAVNKVMVPMGALQPGL | |
| | PSPVAIPLNYHKIVIDLKDCFFTIPLHPEDRPYFAFSVPQINFQSPMP | |
| | RYQWKVLPQGMANSPTLCQKFVAAAIAPVRSQWPEAYILHYMDDILLA | |
| | CDSAEAAKACYAHIISCLTSYGLKIAPDKVQVSEPFSYLGFELHHQQV | |
| | FTPRVCLKTDHLKTLNDFQKLLGDIQWLRPYLKLPTSALVPLNNILKG | |
| | DPNPLSVRALTPEAKQSLALINKAIQNQSVQQISYNLPLVLLLLPTPH | |
| | TPTAVFWQPNGTDPTKNGSPLLWLHLPASPSKVLLTYPSLLAMLIIKG | |
| | RYTGRQLFGRDPHSIIIPYTQDQLTWLLQTSDEWAIALSSFTGDIDNH | |
| | YPSDPVIQFAKLHQFIFPKITKCAPIPQATLVFTDGSSNGIAAYVIDN | |
| | QPISIKSPYLSAQLVELYAILQVFTVLAHQPENLYTDSAYIAQSVPLL | |
| | ETVPFIKSSTNATPLFSKLQQLILNRQHPFFIGHLRAHLNLPGPLAEG | |
| | NALADAATQIFPIISD | |
| SMRVH_P03364_ | PRSRAIDIPVPHADKISWKITDPVWVDQWPLTYEKTLAAIALVQEQLA | 452 |
| 2mut | AGHIEPTNSPWNTPIFIIKKKSGSWRLLQDLRAVNKVMVPMGALQPGL | |
| | PSPVAIPLNYHKIVIDLKDCFFTIPLHPEDRPYFAFSVPQINFQSPMP | |
| | RYQWKVLPQGMANSPTLCQKFVAAAIAPVRSQWPEAYILHYMDDILLA | |
| | CDSAEAAKACYAHIISCLTSYGLKIAPDKVQVSEPFSYLGFELHHQQV | |
| | FTPRVCLKTDHLKTLNDFQKLLGDIQWLRPYLKLPTSALVPLNNILKP | |
| | DPNPLSVRALTPEAKQSLALINKAIQNQSVQQISYNLPLVLLLLPTPH | |
| | TPTAVFWQPNGTDPTKNGSPLLWLHLPASPSKVLLTYPSLLAMLIIKG | |
| | RYTGRQLFGRDPHSIIIPYTQDQLTWLLQTSDEWAIALSSFTGDIDNH | |
| | YPSDPVIQFAKLHQFIFPKITKCAPIPQATLVFTDGSSNGIAAYVIDN | |
| | QPISIKSPYLSAQLVELYAILQVFTVLAHQPENLYTDSAYIAQSVPLL | |
| | ETVPFIKSSTNATPLFSKLQQLILNRQHPFFIGHLRAHLNLPGPLAEG | |
| | NALADAATQIFPIISD | |
| SMRVH_P03364_ | PRSRAIDIPVPHADKISWKITDPVWVDQWPLTYEKTLAAIALVQEQLA | 453 |
| 2mutB | AGHIEPTNSPWNTPIFIIKKKSGSWRLLQDLRAVNKVMVPMGALQPGL | |
| | PSPVAPPLNYHKIVIDLKDCFFTIPLHPEDRPYFAFSVPQINFQSPMP | |
| | RYQWKVLPQGMANSPTLCQKFVAAAIAPVRSQWPEAYILHYMDDILLA | |
| | CDSAEAAKACYAHIISCLTSYGLKIAPDKVQVSEPFSYLGFELHHQQV | |
| | FTPRVCLKTDHLKTLNDFQKLLGDIQWLRPYLKLPTSALVPLNNILKP | |
| | DPNPLSVRALTPEAKQSLALINKAIQNQSVQQISYNLPLVLLLLPTPH | |
| | TPTAVFWQPNGTDPTKNGSPLLWLHLPASPSKVLLTYPSLLAMLIIKG | |
| | RYTGRQLFGRDPHSIIIPYTQDQLTWLLQTSDEWAIALSSFTGDIDNH | |
| | YPSDPVIQFAKLHQFIFPKITKCAPIPQATLVFTDGSSNGIAAYVIDN | |
| | QPISIKSPYLSAQLVELYAILQVFTVLAHQPENLYTDSAYIAQSVPLL | |
| | ETVPFIKSSTNATPLFSKLQQLILNRQHPFFIGHLRAHLNLPGPLAEG | |
| | NALADAATQIFPIISD | |
| SRV2_P51517 | LATAVDILAPQRYADPITWKSDEPVWVDQWPLTQEKLAAAQQLVQEQL | 454 |
| | QAGHIIESNSPWNTPIFVIKKKSGKWRLLQDLRAVNATMVLMGALQPG | |
| | LPSPVAIPQGYFKIVIDLKDCFFTIPLQPVDQKRFAFSLPSTNFKQPM | |
| | KRYQWKVLPQGMANSPTLCQKYVAAAIEPVRKSWAQMYIIHYMDDILI | |
| | AGKLGEQVLQCFAQLKQALTTTGLQIAPEKVQLQDPYTYLGFQINGPK | |
| | ITNQKAVIRRDKLQTLNDFQKLLGDINWLRPYLHLTTGDLKPLFDILK | |
| | GDSNPNSPRSLSEAALASLQKVETAIAEQFVTQIDYTQPLTFLIFNTT | |
| | LTPTGLFWQNNPVMWVHLPASPKKVLLPYYDAIADLIILGRDNSKKYF | |
| | GLEPSTIIQPYSKSQIHWLMQNTETWPIACASYAGNIDNHYPPNKLIQ | |
| | FCKLHAVVFPRIISKTPLDNALLVFTDGSSTGIAAYTFEKTTVRFKTS | |
| | HTSAQLVELQALIAVLSAFPHRALNVYTDSAYLAHSIPLLETVSHIKH | |
| | ISDTAKFFLQCQQLIYNRSIPFYLGHIRAHSGLPGPLSQGNHITDLAT | |
| | KVVATTLTT | |
| SRV2_P51517_ | LATAVDILAPQRYADPITWKSDEPVWVDQWPLTQEKLAAAQQLVQEQL | 455 |
| 2mutB | QAGHIIESNSPWNTPIFVIKKKSGKWRLLQDLRAVNATMVLMGALQPG | |
| | LPSPVAPPQGYFKIVIDLKDCFFTIPLQPVDQKRFAFSLPSTNFKQPM | |
| | KRYQWKVLPQGMANSPTLCQKYVAAAIEPVRKSWAQMYIIHYMDDILI | |
| | AGKLGEQVLQCFAQLKQALTTTGLQIAPEKVQLQDPYTYLGFQINGPK | |
| | ITNQKAVIRRDKLQTLNDFQKLLGDINWLRPYLHLTTGDLKPLFDILK | |
| | GDSNPNSPRSLSEAALASLQKVETAIAEQFVTQIDYTQPLTFLIFNTT | |
| | LTPTGLFWQNNPVMWVHLPASPKKVLLPYYDAIADLIILGRDNSKKYF | |
| | GLEPSTIIQPYSKSQIHWLMQNTETWPIACASYAGNIDNHYPPNKLIQ | |
| | FCKLHAVVFPRIISKTPLDNALLVFTDGSSTGIAAYTFEKTTVRFKTS | |
| | HTSAQLVELQALIAVLSAFPHRALNVYTDSAYLAHSIPLLETVSHIKH | |
| | ISDTAKFFLQCQQLIYNRSIPFYLGHIRAHSGLPGPLSQGNHITDLAT | |
| | KVVATTLTT | |
| WDSV_O92815 | SCQTKNTLNIDEYLLQFPDQLWASLPTDIGRMLVPPITIKIKDNASLP | 456 |
| | SIRQYPLPKDKTEGLRPLISSLENQGILIKCHSPCNTPIFPIKKAGRD | |
| | EYRMIHDLRAINNIVAPLTAVVASPTTVLSNLAPSLHWFTVIDLSNAF | |
| | FSVPIHKDSQYLFAFTFEGHQYTWTVLPQGFIHSPTLFSQALYQSLHK | |
| | IKFKISSEICIYMDDVLIASKDRDTNLKDTAVMLQHLASEGHKVSKKK | |
| | LQLCQQEVVYLGQLLTPEGRKILPDRKVTVSQFQQPTTIRQIRAFLGL | |
| | VGYCRHWIPEFSIHSKFLEKQLKKDTAEPFQLDDQQVEAFNKLKHAIT | |
| | TAPVLVVPDPAKPFQLYTSHSEHASIAVLTQKHAGRTRPIAFLSSKFD | |
| | AIESGLPPCLKACASIHRSLTQADSFILGAPLIIYTTHAICTLLQRDR | |
| | SQLVTASRFSKWEADLLRPELTFVACSAVSPAHLYMQSCENNIPPHDC | |
| | VLLTHTISRPRPDLSDLPIPDPDMTLFSDGSYTTGRGGAAVVMHRPVT | |
| | DDFIIIHQQPGGASAQTAELLALAAACHLATDKTVNIYTDSRYAYGVV | |
| | HDFGHLWMHRGFVTSAGTPIKNHKEIEYLLKQIMKPKQVSVIKIEAHT | |
| | KGVSMEVRGNAAADEAAKNAVFLVQR | |
| WDSV_O92815_ | SCQTKNTLNIDEYLLQFPDQLWASLPTDIGRMLVPPITIKIKDNASLP | 457 |
| 2mut | SIRQYPLPKDKTEGLRPLISSLENQGILIKCHSPCNTPIFPIKKAGRD | |
| | EYRMIHDLRAINNIVAPLTAVVASPTTVLSNLAPSLHWFTVIDLSNAF | |
| | FSVPIHKDSQYLFAFTFEGHQYTWTVLPQGFIHSPTLFNQALYQSLHK | |
| | IKFKISSEICIYMDDVLIASKDRDTNLKDTAVMLQHLASEGHKVSKKK | |
| | LQLCQQEVVYLGQLLTPEGRKILPDRKVTVSQFQQPTTIRQIRAFLGL | |
| | VGYCRHWIPEFSIHSKFLEKQLKPDTAEPFQLDDQQVEAFNKLKHAIT | |
| | TAPVLVVPDPAKPFQLYTSHSEHASIAVLTQKHAGRTRPIAFLSSKED | |
| | AIESGLPPCLKACASIHRSLTQADSFILGAPLIIYTTHAICTLLQRDR | |
| | SQLVTASRFSKWEADLLRPELTFVACSAVSPAHLYMQSCENNIPPHDC | |
| | VLLTHTISRPRPDLSDLPIPDPDMTLFSDGSYTTGRGGAAVVMHRPVT | |
| | DDFIIIHQQPGGASAQTAELLALAAACHLATDKTVNIYTDSRYAYGVV | |
| | HDFGHLWMHRGFVTSAGTPIKNHKEIEYLLKQIMKPKQVSVIKIEAHT | |
| | KGVSMEVRGNAAADEAAKNAVELVQR | |
| WDSV_O92815_ | SCQTKNTLNIDEYLLQFPDQLWASLPTDIGRMLVPPITIKIKDNASLP | 458 |
| 2mutA | SIRQYPLPKDKTEGLRPLISSLENQGILIKCHSPCNTPIFPIKKAGRD | |
| | EYRMIHDLRAINNIVAPLTAVVASPTTVLSNLAPSLHWFTVIDLSNAF | |
| | FSVPIHKDSQYLFAFTFEGHQYTWTVLPQGFIHSPTLFNQALYQSLHK | |
| | IKFKISSEICIYMDDVLIASKDRDTNLKDTAVMLQHLASEGHKVSKKK | |
| | LQLCQQEVVYLGQLLTPEGRKILPDRKVTVSQFQQPTTIRQIRAFLGK | |
| | VGYCRHFIPEFSIHSKFLEKQLKPDTAEPFQLDDQQVEAFNKLKHAIT | |
| | TAPVLVVPDPAKPFQLYTSHSEHASIAVLTQKHAGRTRPIAFLSSKED | |
| | AIESGLPPCLKACASIHRSLTQADSFILGAPLIIYTTHAICTLLQRDR | |
| | SQLVTASRFSKWEADLLRPELTFVACSAVSPAHLYMQSCENNIPPHDC | |
| | VLLTHTISRPRPDLSDLPIPDPDMTLFSDGSYTTGRGGAAVVMHRPVT | |
| | DDFIIIHQQPGGASAQTAELLALAAACHLATDKTVNIYTDSRYAYGVV | |
| | HDFGHLWMHRGFVTSAGTPIKNHKEIEYLLKQIMKPKQVSVIKIEAHT | |
| | KGVSMEVRGNAAADEAAKNAVFLVQR | |
| WMSV_P03359 | VLNLEEEYRLHEKPVPSSIDPSWLQLFPTVWAERAGMGLANQVPPVVV | 459 |
| | ELRSGASPVAVRQYPMSKEAREGIRPHIQRFLDLGVLVPCQSPWNTPL | |
| | LPVKKPGTNDYRPVQDLREINKRVQDIHPTVPNPYNLLSSLPPSHTWY | |
| | SVLDLKDAFFCLKLHPNSQPLFAFEWRDPEKGNTGQLTWTRLPQGFKN | |
| | SPTLFDEALHRDLAPFRALNPQVVLLQYVDDLLVAAPTYRDCKEGTQK | |
| | LLQELSKLGYRVSAKKAQLCQKEVTYLGYLLKEGKRWLTPARKATVMK | |
| | IPPPTTPRQVREFLGTAGFCRLWIPGFASLAAPLYPLTKESIPFIWTE | |
| | EHQKAFDRIKEALLSAPALALPDLTKPFTLYVDERAGVARGVLTQTLG | |
| | PWRRPVAYLSKKLDPVASGWPTCLKAVAAVALLLKDADKLTLGQNVTV | |
| | IASHSLESIVRQPPDRWMTNARMTHYQSLLLNERVSFAPPAVLNPATL | |
| | LPVESEATPVHRCSEILAEETGTRRDLKDQPLPGVPAWYTDGSSFIAE | |
| | GKRRAGAAIVDGKRTVWASSLPEGTSAQKAELVALTQALRLAEGKDIN | |
| | IYTDSRYAFATAHIHGAIYKQRGLLTSAGKDIKNKEEILALLEAIHLP | |
| | KRVAIIHCPGHQKGNDPVATGNRRADEAAKQAALSTRVLAETTKP | |
| WMSV_P03359_ | VLNLEEEYRLHEKPVPSSIDPSWLQLFPTVWAERAGMGLANQVPPVVV | 460 |
| 3mut | ELRSGASPVAVRQYPMSKEAREGIRPHIQRFLDLGVLVPCQSPWNTPL | |
| | LPVKKPGTNDYRPVQDLREINKRVQDIHPTVPNPYNLLSSLPPSHTWY | |
| | SVLDLKDAFFCLKLHPNSQPLFAFEWRDPEKGNTGQLTWTRLPQGFKN | |
| | SPTLFNEALHRDLAPFRALNPQVVLLQYVDDLLVAAPTYRDCKEGTQK | |
| | LLQELSKLGYRVSAKKAQLCQKEVTYLGYLLKEGKRWLTPARKATVMK | |
| | IPPPTTPRQVREFLGTAGFCRLWIPGFASLAAPLYPLTKPSIPFIWTE | |
| | EHQKAFDRIKEALLSAPALALPDLTKPFTLYVDERAGVARGVLTQTLG | |
| | PWRRPVAYLSKKLDPVASGWPTCLKAVAAVALLLKDADKLTLGQNVTV | |
| | IASHSLESIVRQPPDRWMTNARMTHYQSLLLNERVSFAPPAVLNPATL | |
| | LPVESEATPVHRCSEILAEETGTRRDLKDQPLPGVPAWYTDGSSFIAE | |
| | GKRRAGAAIVDGKRTVWASSLPEGTSAQKAELVALTQALRLAEGKDIN | |
| | IYTDSRYAFATAHIHGAIYKQRGWLTSAGKDIKNKEEILALLEAIHLP | |
| | KRVAIIHCPGHQKGNDPVATGNRRADEAAKQAALSTRVLAETTKP | |
| WMSV_P03359_ | VLNLEEEYRLHEKPVPSSIDPSWLQLFPTVWAERAGMGLANQVPPVVV | 461 |
| 3mutA | ELRSGASPVAVRQYPMSKEAREGIRPHIQRFLDLGVLVPCQSPWNTPL | |
| | LPVKKPGTNDYRPVQDLREINKRVQDIHPTVPNPYNLLSSLPPSHTWY | |
| | SVLDLKDAFFCLKLHPNSQPLFAFEWRDPEKGNTGQLTWTRLPQGFKN | |
| | SPTLFNEALHRDLAPFRALNPQVVLLQYVDDLLVAAPTYRDCKEGTQK | |
| | LLQELSKLGYRVSAKKAQLCQKEVTYLGYLLKEGKRWLTPARKATVMK | |
| | IPPPTTPRQVREFLGKAGFCRLFIPGFASLAAPLYPLTKPSIPFIWTE | |
| | EHQKAFDRIKEALLSAPALALPDLTKPFTLYVDERAGVARGVLTQTLG | |
| | PWRRPVAYLSKKLDPVASGWPTCLKAVAAVALLLKDADKLTLGQNVTV | |
| | IASHSLESIVRQPPDRWMTNARMTHYQSLLLNERVSFAPPAVLNPATL | |
| | LPVESEATPVHRCSEILAEETGTRRDLKDQPLPGVPAWYTDGSSFIAE | |
| | GKRRAGAAIVDGKRTVWASSLPEGTSAQKAELVALTQALRLAEGKDIN | |
| | IYTDSRYAFATAHIHGAIYKQRGWLTSAGKDIKNKEEILALLEAIHLP | |
| | KRVAIIHCPGHQKGNDPVATGNRRADEAAKQAALSTRVLAETTKP | |
| XMRV6_A1Z651 | TLNIEDEYRLHETSKEPDVPLGSTWLSDFPQAWAETGGMGLAVRQAPL | 462 |
| | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFDEALHRDLADFRIQHPDLILLQYVDDLLLAATSEQDCQRGT | |
| | RALLQTLGNLGYRASAKKAQICQKQVKYLGYLLKEGQRWLTEARKETV | |
| | MGQPTPKTPRQLREFLGTAGFCRLWIPGFAEMAAPLYPLTKTGTLFNW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEKEAPHDCLEILAETHGTRPDLTDQPIPDADYTWYTDGSSF | |
| | LQEGQRRAGAAVTTETEVIWARALPAGTSAQRAELIALTQALKMAEGK | |
| | KLNVYTDSRYAFATAHVHGEIYRRRGLLTSEGREIKNKNEILALLKAL | |
| | FLPKRLSIIHCPGHQKGNSAEARGNRMADQAAREAAMKAVLETSTLL | |
| XMRV6_A1Z651_ | TLNIEDEYRLHETSKEPDVPLGSTWLSDFPQAWAETGGMGLAVRQAPL | 463 |
| 3mut | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLADFRIQHPDLILLQYVDDLLLAATSEQDCQRGT | |
| | RALLQTLGNLGYRASAKKAQICQKQVKYLGYLLKEGQRWLTEARKETV | |
| | MGQPTPKTPRQLREFLGTAGFCRLWIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEKEAPHDCLEILAETHGTRPDLTDQPIPDADYTWYTDGSSF | |
| | LQEGQRRAGAAVTTETEVIWARALPAGTSAQRAELIALTQALKMAEGK | |
| | KLNVYTDSRYAFATAHVHGEIYRRRGWLTSEGREIKNKNEILALLKAL | |
| | FLPKRLSIIHCPGHQKGNSAEARGNRMADQAAREAAMKAVLETSTLL | |
| XMRV6_A1Z651_ | TLNIEDEYRLHETSKEPDVPLGSTWLSDFPQAWAETGGMGLAVRQAPL | 464 |
| 3mutA | IIPLKATSTPVSIKQYPMSQEARLGIKPHIQRLLDQGILVPCQSPWNT | |
| | PLLPVKKPGTNDYRPVQDLREVNKRVEDIHPTVPNPYNLLSGLPPSHQ | |
| | WYTVLDLKDAFFCLRLHPTSQPLFAFEWRDPEMGISGQLTWTRLPQGF | |
| | KNSPTLFNEALHRDLADFRIQHPDLILLQYVDDLLLAATSEQDCQRGT | |
| | RALLQTLGNLGYRASAKKAQICQKQVKYLGYLLKEGQRWLTEARKETV | |
| | MGQPTPKTPRQLREFLGKAGFCRLFIPGFAEMAAPLYPLTKPGTLENW | |
| | GPDQQKAYQEIKQALLTAPALGLPDLTKPFELFVDEKQGYAKGVLTQK | |
| | LGPWRRPVAYLSKKLDPVAAGWPPCLRMVAAIAVLTKDAGKLTMGQPL | |
| | VILAPHAVEALVKQPPDRWLSNARMTHYQAMLLDTDRVQFGPVVALNP | |
| | ATLLPLPEKEAPHDCLEILAETHGTRPDLTDQPIPDADYTWYTDGSSF | |
| | LQEGQRRAGAAVTTETEVIWARALPAGTSAQRAELIALTQALKMAEGK | |
| | KLNVYTDSRYAFATAHVHGEIYRRRGWLTSEGREIKNKNEILALLKAL | |
| | FLPKRLSIIHCPGHQKGNSAEARGNRMADQAAREAAMKAVLETSTLL | |
| Marathon RT | MDTSNLMEQILSSDNLNRAYLQVVRNKGAEGVDGMKYTELKEHLAKNG | 464 |
| Group II Intron | ETIKGQLRTRKYKPQPARRVEIPKPDGGVRNLGVPTVTDRFIQQAIAQ | |
| CBK9229 | VLTPIYEEQFHDHSYGFRPNRCAQQAILTALNIMNDGNDWIVDIDLEK | |
| | FFDTVNHDKLMTLIGRTIKDGDVISIVRKYLVSGIMIDDEYEDSIVGT | |
| | PQGGNLSPLLANIMLNELDKEMEKRGLNFVRYADDCIIMVGSEMSANR | |
| | VMRNISRFIEEKLGLKVNMTKSKVDRPSGLKYLGFGFYFDPRAHQFKA | |
| | KPHAKSVAKFKKRMKELTCRSWGVSNSYKVEKLNQLIRGWINYFKIGS | |
| | MKTLCKELDSRIRYRLRMCIWKQWKTPQNQEKNLVKLGIDRNTARRVA | |
| | YTGKRIAYVCNKGAVNVAISNKRLASFGLISMLDYYIEKCVTC | |
| TGIRT, trt | MALLERILADRNLITALKRVEANQGAPGIGDVSTDQLRDIYRAHWSTI | 466 |
| Group II Intron | RAQLLAGTYRPAPVRRVGIPKGPGGTRQLGITPVVDRLIQQIALQELT | |
| AAAT7232 | PIFDPDFSPSSFGFRPGRNAHDAVRQAQGYIQEYGRYVVDMDLKEFFD | |
| | RVNHDLIMSRVARKVDKKRVLKLIRYALQAGVMIEGVKVQTEEGTQPG | |
| | GPLSPLLANILLDDLDKELEKRGLKFCYRADDCNIYVSKLRAGQRVKQ | |
| | SIQRFLEKTLKLKVNEEKSVADRPWKRAFGLFSFTPERKARIRLAPRS | |
| | IQRLKQRIRQLTNPNWSISMPREIHRVNQYVGMWIGYFRLVTEPSVLQ | |
| | TIEGWIRRRLRLCWQLQWKRVRTRIRELRALGLKETAVMEIANRTKGA | |
| | WRTTKPQTLHQALGKYTWTAQGLKTSLQRYFELRQG | |
| LtrA | MKPTMAILERISKNSQENIDEVFTRLYRYLLRPDIYYVAYQNLYSNKG | 467 |
| Group II Intron | ASTKGILDDTADGFSEEKIKKIIQSLKDGTYYPQPVRRMYIAKKNSKK | |
| AAB0650 | MRPLGIPTFTDKLIQEAVRIILESIYEPVFEDVSHGFRPQRSCHTALK | |
| | TIKREFGGARWFVEGDIKGCFDNIDHVTLIGLINLKIKDMKMSQLIYK | |
| | FLKAGYLENWQYHKTYSGTPQGGILSPLLANIYLHELDKFVLQLKMKF | |
| | DRESPERITPEYRELHNEIKRISHRLKKLEGEEKAKVLLEYQEKRKRL | |
| | PTLPCTSQTNKVLKYVRYADDFIISVKGSKEDCQWIKEQLKLFIHNKL | |
| | KMELSEEKTLITHSSQPARFLGYDIRVRRSGTIKRSGKVKKRTLNGSV | |
| | ELLIPLQDKIRQFIFDKKIAIQKKDSSWFPVHRKYLIRSTDLEIITIY | |
| | NSELRGICNYYGLASNFNQLNYFAYLMEYSCLKTIASKHKGTLSKTIS | |
| | MFKDGSGSWGIPYEIKQGKQRRYFANFSECKSPYQFTDEISQAPVLYG | |
| | YARNTLENRLKAKCCELCGTSDENTSYEIHHVNKVKNLKGKEKWEMAM | |
| | IAKQRKTLVVCFHCHRHVIHKHK | |
| R2Bm | MMASTALSLMGRCNPDGCTRGKHVTAAPMDGPRGPSSLAGTFGWGLAI | 468 |
| Non-LTR | PAGEPCGRVCSPATVGFFPVAKKSNKENRPEASGLPLESERTGDNPTV | |
| Retrotransposon | RGSAGADPVGQDAPGWTCQFCERTFSTNRGLGVHKRRAHPVETNTDAA | |
| AAB59214.1 | PMMVKRRWHGEEIDLLARTEARLLAERGQCSGGDLFGALPGFGRTLEA | |
| | IKGQRRREPYRALVQAHLARFGSQPGPSSGGCSAEPDFRRASGAEEAG | |
| | EERCAEDAAAYDPSAVGQMSPDAARVLSELLEGAGRRRACRAMRPKTA | |
| | GRRNDLHDDRTASAHKTSRQKRRAEYARVQELYKKCRSRAAAEVIDGA | |
| | CGGVGHSLEEMETYWRPILERVSDAPGPTPEALHALGRAEWHGGNRDY | |
| | TQLWKPISVEEIKASRFDWRTSPGPDGIRSGQWRAVPVHLKAEMFNAW | |
| | MARGEIPEILRQCRTVFVPKVERPGGPGEYRPISIASIPLRHFHSILA | |
| | RRLLACCPPDARQRGFICADGTLENSAVLDAVLGDSRKKLRECHVAVL | |
| | DFAKAFDTVSHEALVELLRLRGMPEQFCGYIAHLYDTASTTLAVNNEM | |
| | SSPVKVGRGVRQGDPLSPILFNVVMDLILASLPERVGYRLEMELVSAL | |
| | AYADDLVLLAGSKVGMQESISAVDCVGRQMGLRLNCRKSAVLSMIPDG | |
| | HRKKHHYLTERTFNIGGKPLRQVSCVERWRYLGVDFEASGCVTLEHSI | |
| | SSALNNISRAPLKPQQRLEILRAHLIPRFQHGFVLGNISDDRLRMLDV | |
| | QIRKAVGQWLRLPADVPKAYYHAAVQDGGLAIPSVRATIPDLIVRRFG | |
| | GLDSSPWSVARAAAKSDKIRKKLRWAWKQLRRFSRVDSTTQRPSVRLF | |
| | WREHLHASVDGRELRESTRTPTSTKWIRERCAQITGRDFVQFVHTHIN | |
| | ALPSRIRGSRGRRGGGESSLTCRAGCKVRETTAHILQQCHRTHGGRIL | |
| | RHNKIVSFVAKAMEENKWTVELEPRLRTSVGLRKPDIIASRDGVGVIV | |
| | DVQVVSGQRSLDELHREKRNKYGNHGELVELVAGRLGLPKAECVRATS | |
| | CTISWRGVWSLTSYKELRSIIGLREPTLQIVPILALRGSHMNWTRENQ | |
| | MTSVMGGGVG | |
| LINE-1 | MTGSNSHITILTLNVNGLNSPIKRHRLASWIKSQDPSVCCIQETHLTC | 469 |
| Non-LTR | RDTHRLKIKGWRKIYQANGKQKKAGVAILVSDKTDFKPTKIKRDKEGH | |
| Retrotransposon | YIMVKGSIQQEELTILNIYAPNTGAPRFIKQVLSDLQRDLDSHTLIMG | |
| AAC5127 | DFNTPLSILDRSTRQKVNKDTQELNSALHQTDLIDIYRTLHPKSTEYT | |
| | FFSAPHHTYSKIDHIVGSKALLSKCKRTEIITNYLSDHSAIKLELRIK | |
| | NLTQSRSTTWKLNNLLLNDYWVHNEMKAEIKMFFETNENKDTTYQNLW | |
| | DAFKAVCRGKFIALNAYKRKQERSKIDTLTSQLKELEKQEQTHSKASR | |
| | RQEITKIRAELKEIETQKTLQKINESRSWFFERINKIDRPLARLIKKK | |
| | REKNQIDTIKNDKGDITTDPTEIQTTIREYYKHLYANKLENLEEMDTF | |
| | LDTYTLPRLNQEEVESLNRPITGSEIVAIINSLPTKKSPGPDGFTAEF | |
| | YQRYKEELVPFLLKLFQSIEKEGILPNSFYEASIILIPKPGRDTTKKE | |
| | NFRPISLMNIDAKILNKILANRIQQHIKKLIHHDQVGFIPGMQGWENI | |
| | RKSINVIQHINRAKDKNHVIISIDAEKAFDKIQQPFMLKTLNKLGIDG | |
| | MYLKIIRAIYDKPTANIILNGQKLEAFPLKTGTRQGCPLSPLLFNIVL | |
| | EVLARAIRQEKEIKGIQLGKEEVKLSLFADDMIVYLENPIVSAQNLLK | |
| | LISNFSKVSGYKINVQKSQAFLYNNNRQTESQIMGELPFTIASKRIKY | |
| | LGIQLTRDVKDLFKENYKPLLKEIKEDTNKWKNIPCSWVGRINIVKMA | |
| | ILPKVIYRFNAIPIKLPMTFFTELEKTTLKFIWNQKRARIAKSILSQK | |
| | NKAGGITLPDFKLYYKATVTKTAWYWYQNRDIDQWNRTEPSEIMPHIY | |
| | NYLIFDKPEKNKQWGKDSLLNKWCWENWLAICRKLKLDPFLTPYTKIN | |
| | SRWIKDLNVKPKTIKTLEENLGITIQDIGVGKDFMSKTPKAMATKDKI | |
| | DKWDLIKLKSFCTAKETTIRVNRQPTTWEKIFATYSSDKGLISRIYNE | |
| | LKQIYKKKTNNPIKKWAKDMNRHFSKEDIYAAKKHMKKCSSSLAIREM | |
| | QIKTTMRYHLTPVRMAIIKKSGNNRCWRGCGEIGTLVHCWWDCKLVQP | |
| | LWKSVWRFLRDLELEIPFDPAIPLLGIYPKDYKSCCYKDTCTRMFIAA | |
| | LFTIAKTWNQPNCPTMIDWIKKMWHIYTMEYYAAIKNDEFISFVGTWM | |
| | KLETIILSKLSQEQKTKHRIFSLIGGN | |
| Penelope | MTGSNSHITILTLNVNGLNSPIKRHRLASWIKSQDPSVCCIQETHLTC | 470 |
| Non-LTR | RDTHRLKIKGWRKIYQANGKQKKAGVAILVSDKTDFKPTKIKRDKEGH | |
| Retrotransposon | YIMVKGSIQQEELTILNIYAPNTGAPRFIKQVLSDLQRDLDSHTLIMG | |
| AAL14979.1 | DENTPLSILDRSTRQKVNKDTQELNSALHQTDLIDIYRTLHPKSTEYT | |
| | FFSAPHHTYSKIDHIVGSKALLSKCKRTEIITNYLSDHSAIKLELRIK | |
| | NLTQSRSTTWKLNNLLLNDYWVHNEMKAEIKMFFETNENKDTTYQNLW | |
| | DAFKAVCRGKFIALNAYKRKQERSKIDTLTSQLKELEKQEQTHSKASR | |
| | RQEITKIRAELKEIETQKTLQKINESRSWFFERINKIDRPLARLIKKK | |
| | REKNQIDTIKNDKGDITTDPTEIQTTIREYYKHLYANKLENLEEMDTF | |
| | LDTYTLPRLNQEEVESLNRPITGSEIVAIINSLPTKKSPGPDGFTAEF | |
| | YQRYKEELVPFLLKLFQSIEKEGILPNSFYEASIILIPKPGRDTTKKE | |
| | NFRPISLMNIDAKILNKILANRIQQHIKKLIHHDQVGFIPGMQGWENI | |
| | RKSINVIQHINRAKDKNHVIISIDAEKAFDKIQQPFMLKTLNKLGIDG | |
| | MYLKIIRAIYDKPTANIILNGQKLEAFPLKTGTRQGCPLSPLLFNIVL | |
| | EVLARAIRQEKEIKGIQLGKEEVKLSLFADDMIVYLENPIVSAQNLLK | |
| | LISNFSKVSGYKINVQKSQAFLYNNNRQTESQIMGELPFTIASKRIKY | |
| | LGIQLTRDVKDLFKENYKPLLKEIKEDINKWKNIPCSWVGRINIVKMA | |
| | ILPKVIYRFNAIPIKLPMTFFTELEKTTLKFIWNQKRARIAKSILSQK | |
| | NKAGGITLPDFKLYYKATVTKTAWYWYQNRDIDQWNRTEPSEIMPHIY | |
| | NYLIFDKPEKNKQWGKDSLLNKWCWENWLAICRKLKLDPFLTPYTKIN | |
| | SRWIKDLNVKPKTIKTLEENLGITIQDIGVGKDFMSKTPKAMATKDKI | |
| | DKWDLIKLKSFCTAKETTIRVNRQPTTWEKIFATYSSDKGLISRIYNE | |
| | LKQIYKKKTNNPIKKWAKDMNRHFSKEDIYAAKKHMKKCSSSLAIREM | |
| | QIKTTMRYHLTPVRMAIIKKSGNNRCWRGCGEIGTLVHCWWDCKLVQP | |
| | LWKSVWRFLRDLELEIPFDPAIPLLGIYPKDYKSCCYKDTCTRMFIAA | |
| | LFTIAKTWNQPNCPTMIDWIKKMWHIYTMEYYAAIKNDEFISFVGTWM | |
| | KLETIILSKLSQEQKTKHRIFSLIGGN | |
| Ty1 | AVKAVKSIKPIRTTLRYDEAITYNKDIKEKEKYIEAYHKEVNQLLKMK | 471 |
| LTR | TWDTDEYYDRKEIDPKRVINSMFIFNKKRDGTHKARFVARGDIQHPDT | |
| Retrotransposon | YDSGMQSNTVHHYALMTSLSLALDNNYYITQLDISSAYLYADIKEELY | |
| AAA6693 | IRPPPHLGMNDKLIRLKKSLYGLKQSGANWYETIKSYLIQQCGMEEVR | |
| | GWSCVFKNSQVTICLFVDDMVLFSKNLNSNKRIIEKLKMQYDTKIINL | |
| | GESDEEIQYDILGLEIKYQRGKYMKLGMENSLTEKIPKLNVPLNPKGR | |
| | KLSAPGQPGLYIDQDELEIDEDEYKEKVHEMQKLIGLASYVGYKFRED | |
| | LLYYINTLAQHILFPSRQVLDMTYELIQFMWDTRDKQLIWHKNKPTEP | |
| | DNKLVAISDASYGNQPYYKSQIGNIYLLNGKVIGGKSTKASLTCTSTT | |
| | EAEIHAISESVPLLNNLSYLIQELNKKPIIKGLLTDSRSTISIIKSTN | |
| | EEKFRNRFFGTKAMRLRDEVSGNNLYVYYIETKKNIADVMTKPLPIKT | |
| | FKLLTNKWIH | |
| Brt | MGKRHRNLIDQITTWENLLDAYRKTSHGKRRTWGYLEFKEYDLANLLA | 472 |
| Q775D8 | LQAELKAGNYERGPYREFLVYEPKPRLISALEFKDRLVQHALCNIVAP | |
| | IFEAGLLPYTYACRPDKGTHAGVCHVQAELRRTRATHFLKSDFSKFFP | |
| | SIDRAALYAMIDKKIHCAATRRLLRVVLPDEGVGIPIGSLTSQLFANV | |
| | YGGAVDRLLHDELKQRHWARYMDDIVVLGDDPEELRAVFYRLRDFASE | |
| | RLGLKISHWQVAPVSRGINFLGYRIWPTHKLLRKSSVKRAKRKVANFI | |
| | KHGEDESLQRFLASWSGHAQWADTHNLFTWMEEQYGIACH | |
| RT86 | MKSAEYLNTFRLRNLGLPVMNNLHDMSKATRISVETLRLLIYTADFRY | 473 |
| P23070 | RIYTVEKKGPEKRMRTIYQPSRELKALQGWVLRNILDKLSSSPFSIGF | |
| | EKHQSILNNATPHIGANFILNIDLEDFFPSLTANKVFGVFHSLGYNRL | |
| | ISSVLTKICCYKNLLPQGAPSSPKLANLICSKLDYRIQGYAGSRGLIY | |
| | TRYADDLTLSAQSMKKVVKARDFLFSIIPSEGLVINSKKTCISGPRSQ | |
| | RKVTGLVISQEKVGIGREKYKEIRAKIHHIFCGKSSEIEHVRGWLSFI | |
| | LSVDSKSHRRLITYISKLEKKYGKNPLNKAKT | |
| TERT | MPRAPRCRAVRSLLRSHYREVLPLATFVRRLGPQGWRLVQRGDPAAFR | 474 |
| O14746 | ALVAQCLVCVPWDARPPPAAPSFRQVSCLKELVARVLQRLCERGAKNV | |
| | LAFGFALLDGARGGPPEAFTTSVRSYLPNTVTDALRGSGAWGLLLRRV | |
| | GDDVLVHLLARCALFVLVAPSCAYQVCGPPLYQLGAATQARPPPHASG | |
| | PRRRLGCERAWNHSVREAGVPLGLPAPGARRRGGSASRSLPLPKRPRR | |
| | GAAPEPERTPVGQGSWAHPGRTRGPSDRGFCVVSPARPAEEATSLEGA | |
| | LSGTRHSHPSVGRQHHAGPPSTSRPPRPWDTPCPPVYAETKHFLYSSG | |
| | DKEQLRPSFLLSSLRPSLTGARRLVETIFLGSRPWMPGTPRRLPRLPQ | |
| | RYWQMRPLFLELLGNHAQCPYGVLLKTHCPLRAAVTPAAGVCAREKPQ | |
| | GSVAAPEEEDTDPRRLVQLLRQHSSPWQVYGFVRACLRRLVPPGLWGS | |
| | RHNERRFLRNTKKFISLGKHAKLSLQELTWKMSVRDCAWLRRSPGVGC | |
| | VPAAEHRLREEILAKFLHWLMSVYVVELLRSFFYVTETTFQKNRLFFY | |
| | RKSVWSKLQSIGIRQHLKRVQLRELSEAEVRQHREARPALLTSRLRFI | |
| | PKPDGLRPIVNMDYVVGARTFRREKRAERLTSRVKALFSVLNYERARR | |
| | PGLLGASVLGLDDIHRAWRTFVLRVRAQDPPPELYFVKVDVTGAYDTI | |
| | PQDRLTEVIASIIKPQNTYCVRRYAVVQKAAHGHVRKAFKSHVSTLTD | |
| | LQPYMRQFVAHLQETSPLRDAVVIEQSSSLNEASSGLFDVFLREMCHH | |
| | AVRIRGKSYVQCQGIPQGSILSTLLCSLCYGDMENKLFAGIRRDGLLL | |
| | RLVDDFLLVTPHLTHAKTFLRTLVRGVPEYGCVVNLRKTVVNFPVEDE | |
| | ALGGTAFVQMPAHGLFPWCGLLLDTRTLEVQSDYSSYARTSIRASLTF | |
| | NRGFKAGRNMRRKLFGVLRLKCHSLFLDLQVNSLQTVCTNIYKILLLQ | |
| | AYRFHACVLQLPFHQQVWKNPTFFLRVISDTASLCYSILKAKNAGMSL | |
| | GAKGAAGPLPSEAVQWLCHQAFLLKLTRHRVTYVPLLGSLRTAQTQLS | |
| | RKLPGTTLTALEAAANPALPSDFKTILD | |
| Mauriceville | MPNHRLPNCVSYLGENHELSWLHGMFGLLKRSNPQTGGILGWLNTGPN | 475 |
| Q36578 | GFVKYMMNLMGHARDKGDAKEYWRLGRSLMKNEAFQVQAFNHVCKHWY | |
| | LDYKPHKIAKLLKEVREMVEIQPVCIDYKRVYIPKANGKQRPLGVPTV | |
| | PWRVYLHMWNVLLVWYRIPEQDNQHAYFPKRGVFTAWRALWPKLDSQN | |
| | IYEFDLKNFFPSVDLAYLKDKLMESGIPQDISEYLTVLNRSLVVLTSE | |
| | DKIPEPHRDVIFNSDGTPNPNLPKDVQGRILKDPDFVEILRRRGFTDI | |
| | ATNGVPQGASTSCGLATYNVKELFKRYDELIMYADDGILCRQDPSTPD | |
| | FSVEEAGVVQEPAKSGWIKQNGEFKKSVKFLGLEFIPANIPPLGEGEV | |
| | KDYPRLRGATRNGSKMELSTELQFLCYLSYKLRIKVLRDLYIQVLGYL | |
| | PSVPLLRYRSLAEAINELSPKRITIGQFITSSFEEFTAWSPLKRMGFF | |
| | FSSPAGPTILSSIFNNSTNLQEPSDSRLLYRKGSWVNIRFAAYLYSKL | |
| | SEEKHGLVPKFLEKLREINFALDKVDVTEIDSKLSRLMKFSVSAAYDE | |
| | VGTLALKSLFKFRNSERESIKASFKQLRENGKIAEFSEARRLWFEILK | |
| | LIRLDLFNASSLACDDLLSHLQDRRSIKKWGSSDVLYLKSQRLMRINK | |
| | KQLQLDFEKKKNSLKKKLIKRRAKELRDTFKGKENKEA | |
| RTX | MILDTDYITEDGKPVIRIFKKENGEFKIEYDRTFEPYLYALLKDDSAI | 476 |
| QFN49000.1 | EEVKKITAERHGTVVTVKRVEKVQKKFLGRPVEVWKLYFTHPQDVPAI | |
| | MDKIREHPAVIDIYEYDIPFAIRYLIDKGLVPMEGDEELKLLAFDIET | |
| | LYHEGEEFAEGPILMISYADEEGARVITWKNVDLPYVDVVSTEREMIK | |
| | RFLRVVKEKDPDVLITYNGDNFDFAYLKKRCEKLGINFALGRDGSEPK | |
| | IQRMGDRFAVEVKGRIHFDLYPVIRRTINLPTYTLEAVYEAVFGQPKE | |
| | KVYAEEITTAWETGENLERVARYSMEDAKVTYELGKEFLPMEAQLSRL | |
| | IGQSLWDVSRSSTGNLVEWELLRKAYERNELAPNKPDEKELARRHQSH | |
| | EGGYIKEPERGLWENIVYLDERSLYPSIIITHNVSPDTLNREGCKEYD | |
| | VAPQVGHRFCKDFPGFIPSLLGDLLEERQKIKKRMKATIDPIERKLLD | |
| | YRQRAIKILANSLYGYYGYARARWYCKECAESVIAWGREYLTMTIKEI | |
| | EEKYGFKVIYSDTDGFFATIPGADAETVKKKAMEFLKYINAKLPGALE | |
| | LEYEGFYKRGLFVTKKKYAVIDEEGKITTRGLEIVRRDWSEIAKETQA | |
| | RVLEALLKDGDVEKAVRIVKEVTEKLSKYEVPPEKLVIHKQITRDLKD | |
| | YKATGPHVAVAKRLAARGVKIRPGTVISYIVLKGSGRIVDRAIPFDEF | |
| | DPTKHKYDAEYYIEKQVLPAVERILRAFGYRKEDLRYQKTRQVGLSAR | |
| | LKPKGTLEGSSHHHHHH | |

In some embodiments, the amino acid sequence of the reverse transcriptase (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 2.

In some embodiments, the amino acid sequence of the reverse transcriptase (or the functional fragment, functional variant, or domain thereof) comprises or consists of the amino acid sequence of a polypeptide set forth in Table 2, and further comprises 1 or more but less than 15% (e.g., less than 12%, less than 10%, less than 8%), amino acid variations (e.g., substitutions, additions, deletions, etc.). In some embodiments, the amino acid sequence of the reverse transcriptase (or the functional fragment, functional variant, or domain thereof) comprises or consists of the amino acid sequence of a polypeptide set forth in Table 2, and further comprises or consists of at least about 1, 2, 3, 4, 5, 6, 7, 8, 9, or 10 amino acid variations (e.g., substitutions, additions, deletions, etc.). In some embodiments, the amino acid sequence of the reverse transcriptase ((or the functional fragment, functional variant, or domain thereof) comprises or consists of the amino acid sequence of a polypeptide set forth in Table 2, and further comprises or consists of about 1, 2, 3, 4, 5, 6, 7, 8, 9, or 10 amino acid variations (e.g., substitutions, additions, deletions, etc.). In some embodiments, the amino acid sequence of the reverse transcriptase (or the functional fragment, functional variant, or domain thereof) comprises or consists of the amino acid sequence of a polypeptide set forth in Table 2, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the reverse transcriptase (or the functional fragment, functional variant, or domain thereof) comprises or consists of the amino acid sequence of a polypeptide set forth in Table 2, and further comprises 1 or more but less than 15% (e.g., less than 12%, less than 10%, less than 8%), amino acid substitutions. In some embodiments, the amino acid sequence of the reverse transcriptase (or the functional fragment, functional variant, or domain thereof) comprises or consists of the amino acid sequence of a polypeptide set forth in Table 2, and further comprises or consists of at least about 1, 2, 3, 4, 5, 6, 7, 8, 9, or 10 amino acid substitutions. In some embodiments, the amino acid sequence of the reverse transcriptase (or the functional fragment or variant thereof) comprises or consists of the amino acid sequence of a polypeptide set forth in Table 2, and further comprises or consists of about 1, 2, 3, 4, 5, 6, 7, 8, 9, or 10 amino acid substitutions. In some embodiments, the amino acid sequence of the reverse transcriptase (or the functional fragment, functional variant, or domain thereof) comprises or consists of the amino acid sequence of a polypeptide set forth in Table 2, and further comprises or consists of no more than about 1, 2, 3, 4, 5, 6, 7, 8, 9, or 10 amino acid substitutions.

In some embodiments, the amino acid sequence of reverse transcriptase (or the functional fragment or variant thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 324-476.

In some embodiments, the amino acid sequence of the reverse transcriptase (or the functional fragment, functional variant, or domain thereof) comprises or consists of the amino acid sequence of any one of SEQ ID NOS: 324-476, and further comprises 1 or more but less than 15% (less than 12%, less than 10%, less than 8%), amino acid variations (e.g., substitutions, additions, deletions, etc.). In some embodiments, the amino acid sequence of the reverse transcriptase (or the functional fragment, functional variant, or domain thereof) comprises or consists of the amino acid sequence of any one of SEQ ID NOS: 324-476, and further comprises or consists of at least about 1, 2, 3, 4, 5, 6, 7, 8, 9, or 10 amino acid variations (e.g., substitutions, additions, deletions, etc.). In some embodiments, the amino acid sequence of the reverse transcriptase (or the functional fragment or variant thereof) comprises or consists of the amino acid sequence of any one of SEQ ID NOS: 324-476, and further comprises or consists of about 1, 2, 3, 4, 5, 6, 7, 8, 9, or 10 amino acid variations (e.g., substitutions, additions, deletions, etc.). In some embodiments, the amino acid sequence of the reverse transcriptase (or the functional fragment, functional variant, or domain thereof) comprises or consists of the amino acid sequence of any one of SEQ ID NOS: 324-476, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the reverse transcriptase (or the functional fragment, functional variant, or domain thereof) comprises or consists of the amino acid sequence of any one of SEQ ID NOS: 324-476, and further comprises 1 or more but less than 15% (e.g., less than 12%, less than 10%, less than 8%), amino acid substitutions. In some embodiments, the amino acid sequence of the reverse transcriptase (or the functional fragment, functional variant, or domain thereof) comprises or consists of the amino acid sequence of any one of SEQ ID NOS: 324-476, and further comprises or consists of at least about 1, 2, 3, 4, 5, 6, 7, 8, 9, or 10 amino acid substitutions. In some embodiments, the amino acid sequence of the reverse transcriptase (or the functional fragment, functional variant, or domain thereof) comprises or consists of the amino acid sequence of any one of SEQ ID NOS: 324-476, and further comprises or consists of about 1, 2, 3, 4, 5, 6, 7, 8, 9, or 10 amino acid substitutions. In some embodiments, the amino acid sequence of the reverse transcriptase (or the functional fragment, functional variant, or domain thereof) comprises or consists of the amino acid sequence of any one of SEQ ID NOS: 324-476, and further comprises or consists of no more than about 1, 2, 3, 4, 5, 6, 7, 8, 9, or 10 amino acid substitutions.

In some embodiments, the RT is a RT (or a functional fragment, functional variant, or domain thereof) described in WO2021178720 (see, e.g., Table 1, Table 2, Table 3, Table 30, Table 41, Table 44) and WO2023039424 (see, e.g., Table 6), the entire contents of which are incorporated herein by reference for all purposes.

In some embodiments, the amino acid sequence of the reverse transcriptase (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide described in WO2021178720 (see, e.g., Table 1, Table 2, Table 3, Table 30, Table 41, Table 44) and WO2023039424 (see, e.g., Table 6).

In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises 1 or more but less than 15% (e.g., less than 12%, less than 10%, less than 8%), amino acid variations (e.g., substitutions, additions, deletions, etc.). In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises or consists of at least about 1, 2, 3, 4, 5, 6, 7, 8, 9, or 10 amino acid variations (e.g., substitutions, additions, deletions, etc.). In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises or consists of about 1, 2, 3, 4, 5, 6, 7, 8, 9, or 10 amino acid variations (e.g., substitutions, additions, deletions, etc.). In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises or consists of no more than about 1, 2, 3, 4, 5, 6, 7, 8, 9, or 10 amino acid variations (e.g., substitutions, additions, deletions, etc.).

In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises 1 or more but less than 15% (e.g., less than 12%, less than 10%, less than 8%), amino acid substitutions. In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises or consists of at least about 1, 2, 3, 4, 5, 6, 7, 8, 9, or 10 amino acid substitutions. In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises or consists of about 1, 2, 3, 4, 5, 6, 7, 8, 9, or 10 amino acid substitutions. In some embodiments, the amino acid sequence of the reverse transcriptase (or the functional fragment, functional variant, or domain thereof) comprises or consists of the amino acid sequence of a polypeptide described in WO2021178720 (see, e.g., Table 1, Table 2, Table 3, Table 30, Table 41, Table 44), and further comprises or consists of no more than about 1, 2, 3, 4, 5, 6, 7, 8, 9, or 10 amino acid substitutions.

### 4.3.1.2 Nucleobase Editors

In some embodiments, the heterologous protein (or a functional fragment, functional variant, or domain thereof) exhibits nucleobase editing activity. In some embodiments, the heterologous protein (or a functional fragment, functional variant, or domain thereof) comprises or consists of the nucleobase editing domain (e.g., a domain capable of modifying a nucleobase (e.g., A, T, C, G, or U) within a nucleic acid molecule (e.g., DNA)) of a nucleobase editor (e.g., a nucleobase editor described herein).

In some embodiments, the heterologous protein is a nucleobase editor (or a functional fragment, functional variant, or domain thereof). In some embodiments, the nucleobase editor (or the functional fragment, functional variant, or domain thereof) comprises or consists of the nucleobase editing domain (e.g., a domain capable of modifying a base (e.g., A, T, C, G, or U) within a nucleic acid molecule (e.g., DNA)) of a nucleobase editor (e.g., a nucleobase editor described herein). In some embodiments, the nucleobase editor is a deaminase (or a functional fragment, functional variant, or domain thereof). In some embodiments, the deaminase is a cytidine deaminase (or a functional fragment, functional variant, or domain thereof). In some embodiments, the deaminase is an adenosine deaminase (or a functional fragment, functional variant, or domain thereof).

In some embodiments, the nucleobase editor comprises a naturally occurring nucleobase editor (e.g., deaminase) (or the functional fragment, functional variant, or domain thereof). In some embodiments, the nucleobase editor (e.g., deaminase) comprises a functional fragment of a naturally occurring nucleobase editor. In some embodiments, the nucleobase editor (e.g., deaminase) comprises a functional variant of a naturally occurring nucleobase editor. In some embodiments, the nucleobase editor (e.g., deaminase) comprises a functional fragment and variant of a naturally occurring nucleobase editor. In some embodiments, the nucleobase editor (e.g., deaminase) comprises one or more domain of a naturally occurring nucleobase editor. In some embodiments, the nucleobase editor (e.g., deaminase) comprises a functional fragment of one or more domain of a naturally occurring nucleobase editor. In some embodiments, the nucleobase editor (e.g., deaminase) comprises a functional variant of one or more domain of a naturally occurring nucleobase editor. In some embodiments, the nucleobase editor (e.g., deaminase) comprises a functional fragment and functional variant of one or more domain of a naturally occurring nucleobase editor.

In some embodiments, the nucleobase editor (e.g., deaminase) is a eukaryotic nucleobase editor (or the functional fragment, functional variant, or domain thereof). In some embodiments, the nucleobase editor (e.g., deaminase) is a prokaryotic nucleobase editor (or the functional fragment, functional variant, or domain thereof). In some embodiments, the nucleobase editor (e.g., deaminase) is a viral nucleobase editor (or the functional fragment, functional variant, or domain thereof). In some embodiments, the nucleobase editor (e.g., deaminase) is a bacterial nucleobase editor (or the functional fragment, functional variant, or domain thereof).

Naturally occurring nucleobase editors, e.g., deaminases (e.g., cytidine deaminases, adenosine deaminases), are known in the art and described herein (see, e.g., Table 3).

For example, naturally occurring cytidine deaminases include, but are not limited to, the apolipoprotein B mRNA editing complex (APOBEC) family deaminases and cytidine deaminase 1 (CDA1). The APOBEC family includes, for example, but are not limited to, APOBEC1, APOBEC2, APOBEC3A, APOBEC3B, APOBEC3C, APOBEC3D (now typically referred to as “APOBEC3E”), APOBEC3F, APOBEC3G, APOBEC3H, APOBEC4, and activation-induced (cytidine or cytosine) deaminase (AID). The cytidine deaminase can be derived from any suitable organism, including, e.g., human, chimpanzee, gorilla, monkey, cow, dog, rat, or mouse. Exemplary cytidine deaminases are described in WO2022/204268, the entire contents of which is incorporated herein by reference for all purposes.

Naturally occurring adenosine deaminases include, for example, but are not limited to, adenosine deaminase ADAR (e.g., ADAR1, ADAR2), adenosine deaminase ADAT, TadA (e.g., from Escherichia coli (ecTadA)). TadA and variants thereof are known in the art and described in, e.g., WO2018/027078 and WO2022/204268, the entire contents of each of which are incorporated herein by reference for all purposes. The adenosine deaminase can be derived from any suitable organism (e.g., Escherichia coli). In some embodiments, the adenosine deaminase is derived from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is derived from Escherichia coli. In some embodiments, the adenosine deaminase is an ecTadA. In some embodiments, the ecTadA is a variant as described in WO2018/027078 or WO2022/204268, the entire contents of each of which are incorporated herein by reference for all purposes.

In some embodiments, the adenosine deaminase is a variant TadA deaminase. In some embodiments, the variant TadA deaminase is one described in WO2022/204268 (see, e.g., Table 3, pages 91-93), the entire contents of which are incorporated herein by reference for all purposes. In some embodiments, the TadA is provided as a monomer or dimer (e.g., a heterodimer of wild-type E. coli TadA and an engineered TadA variant). In some embodiments, the adenosine deaminase is an eighth generation TadA* 8 variant as described in WO2022/204268 (see, e.g., Table 4). In some embodiments, the adenosine deaminase is an eighth generation TadA* 8 variant as shown in WO2022/204268 (see, e.g., pages 91-92), the entire contents of which are incorporated herein by reference for all purposes.

Exemplary nucleobase editors are described in, e.g., WO2022/204268, WO2018/027078, WO2017/070632, Komor, A. C., et al., “Programmable editing of a target base in genomic DNA without double-stranded DNA cleavage” Nature 533, 420-424 (2016); Gaudelli, N. M., et al., “Programmable base editing of A·T to G»C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of each of which are hereby incorporated herein by reference for all purposes.

The amino acid sequence of exemplary nucleobase editors is provided in Table 3.

TABLE 3
Amino Acid Sequence of Exemplary Nucleobase Editors.

| | | SEQ |
|---|---|---|
| Description | Amino Acid Sequence | ID NO |
| Petromyzon | MTDAEYVRIHEKLDIYTFKKQFFNNKKSVSHRCYVLFELKRRGERRACFW | 477 |
| marinus CDA1 | GYAVNKPQSGTERGIHAEIFSIRKVEEYLRDNPGQFTINWYSSWSPCADC | |
| | AEKILEWYNQELRGNGHTLKIWACKLYYEKNARNQIGLWNLRDNGVGLNV | |
| | MVSEHYQCCRKIFIQSSHNQLNENRWLEKTLKRAEKRRSELSIMIQVKIL | |
| | HTTKSPAV | |
| Human AID | MDSLLMNRRKFLYQFKNVRWAKGRRETYLCYVVKRRDSATSFSLDFGYLR | 478 |
| | NKNGCHVELLFLRYISDWDLDPGRCYRVTWFTSWSPCYDCARHVADFLRG | |
| | NPNLSLRIFTARLYFCEDRKAEPEGLRRLHRAGVQIAIMTFKDYFYCWNT | |
| | FVENHERTFKAWEGLHENSVRLSRQLRRILLPLYEVDDLRDAFRTLGL | |
| Murine AID | MDSLLMKQKKFLYHFKNVRWAKGRHETYLCYVVKRRDSATSCSLDFGHLR | 479 |
| | NKSGCHVELLFLRYISDWDLDPGRCYRVTWFTSWSPCYDCARHVAEFLRW | |
| | NPNLSLRIFTARLYFCEDRKAEPEGLRRLHRAGVQIGIMTFKDYFYCWNT | |
| | FVENRERTFKAWEGLHENSVRLTRQLRRILLPLYEVDDLRDAFRMLGF | |
| Canine AID | MDSLLMKQRKFLYHFKNVRWAKGRHETYLCYVVKRRDSATSFSLDFGHLR | 480 |
| | NKSGCHVELLFLRYISDWDLDPGRCYRVTWFTSWSPCYDCARHVADFLRG | |
| | YPNLSLRIFAARLYFCEDRKAEPEGLRRLHRAGVQIAIMTFKDYFYCWNT | |
| | FVENREKTFKAWEGLHENSVRLSRQLRRILLPLYEVDDLRDAFRTLGL | |
| Bovine AID | MDSLLKKQRQFLYQFKNVRWAKGRHETYLCYVVKRRDSPTSFSLDFGHLR | 481 |
| | NKAGCHVELLFLRYISDWDLDPGRCYRVTWFTSWSPCYDCARHVADFLRG | |
| | YPNLSLRIFTARLYFCDKERKAEPEGLRRLHRAGVQIAIMTFKDYFYCWN | |
| | TFVENHERTFKAWEGLHENSVRLSRQLRRILLPLYEVDDLRDAFRTLGL | |
| Rat AID | MAVGSKPKAALVGPHWERERIWCFLCSTGLGTQQTGQTSRWLRPAATQDP | 482 |
| | VSPPRSLLMKQRKFLYHFKNVRWAKGRHETYLCYVVKRRDSATSFSLDFG | |
| | YLRNKSGCHVELLFLRYISDWDLDPGRCYRVTWFTSWSPCYDCARHVADF | |
| | LRGNPNLSLRIFTARLTGWGALPAGLMSPARPSDYFYCWNTEVENHERTE | |
| | KAWEGLHENSVRLSRRLRRILLPLYEVDDLRDAFRTLGL | |
| Canis lupus | MDSLLMKQRKFLYHFKNVRWAKGRHETYLCYVVKRRDSATSFSLDFGHLR | 483 |
| familiaris AID | NKSGCHVELLFLRYISDWDLDPGRCYRVTWFTSWSPCYDCARHVADFLRG | |
| | YPNLSLRIFAARLYFCEDRKAEPEGLRRLHRAGVQIAIMTFKDYFYCWNT | |
| | FVENREKTFKAWEGLHENSVRLSRQLRRILLPLYEVDDLRDAFRTLGL | |
| Bos taurus AID | MDSLLKKQRQFLYQFKNVRWAKGRHETYLCYVVKRRDSPTSFSLDFGHLR | 484 |
| | NKAGCHVELLFLRYISDWDLDPGRCYRVTWFTSWSPCYDCARHVADFLRG | |
| | YPNLSLRIFTARLYFCDKERKAEPEGLRRLHRAGVQIAIMTFKDYFYCWN | |
| | TFVENHERTFKAWEGLHENSVRLSRQLRRILLPLYEVDDLRDAFRTLGL | |
| Mus musculus | MDSLLMNRRKFLYQFKNVRWAKGRRETYLCYVVKRRDSATSFSLDFGYLR | 485 |
| AID | NKNGCHVELLFLRYISDWDLDPGRCYRVTWFTSWSPCYDCARHVADFLRG | |
| | NPNLSLRIFTARLYFCEDRKAEPEGLRRLHRAGVQIAIMTFKDYFYCWNT | |
| | FVENHERTFKAWEGLHENSVRLSRQLRRILLPLYEVDDLRDAFRTLGL | |
| Rattus | MSSETGPVAVDPTLRRRIEPHEFEVFFDPRELRKETCLLYEINWGGRHSI | 486 |
| norvegicus | WRHTSQNTNKHVEVNFIEKFTTERYFCPNTRCSITWFLSWSPCGECSRAI | |
| APOBEC-1 | TEFLSRYPHVTLFIYIARLYHHADPRNRQGLRDLISSGVTIQIMTEQESG | |
| | YCWRNFVNYSPSNEAHWPRYPHLWVRLYVLELYCIILGLPPCLNILRRKQ | |
| | PQLTFFTIALQSCHYQRLPPHILWATGLK | |
| Mesocricetus | MSSETGPVVVDPTLRRRIEPHEFDAFFDQGELRKETCLLYEIRWGGRHNI | 487 |
| auratus | WRHTGQNTSRHVEINFIEKFTSERYFYPSTRCSIVWFLSWSPCGECSKAI | |
| APOBEC-1 | TEFLSGHPNVTLFIYAARLYHHTDQRNRQGLRDLISRGVTIRIMTEQEYC | |
| | YCWRNFVNYPPSNEVYWPRYPNLWMRLYALELYCIHLGLPPCLKIKRRHQ | |
| | YPLTFFRLNLQSCHYQRIPPHILWATGFI | |
| Pongo | MTSEKGPSTGDPTLRRRIESWEFDVFYDPRELRKETCLLYEIKWGMSRKI | 488 |
| pygmaeus | WRSSGKNTINHVEVNFIKKFTSERRFHSSISCSITWFLSWSPCWECSQAI | |
| APOBEC-1 | REFLSQHPGVTLVIYVARLFWHMDQRNRQGLRDLVNSGVTIQIMRASEYY | |
| | HCWRNFVNYPPGDEAHWPQYPPLWMMLYALELHCIILSLPPCLKISRRWQ | |
| | NHLAFFRLHLQNCHYQTIPPHILLATGLIHPSVTWR | |
| Oryctolagus | MASEKGPSNKDYTLRRRIEPWEFEVFFDPQELRKEACLLYEIKWGASSKT | 489 |
| cuniculus | WRSSGKNTTNHVEVNFLEKLTSEGRLGPSTCCSITWFLSWSPCWECSMAI | |
| APOBECI | REFLSQHPGVTLIIFVARLFQHMDRRNRQGLKDLVTSGVTVRVMSVSEYC | |
| | YCWENFVNYPPGKAAQWPRYPPRWMLMYALELYCIILGLPPCLKISRRHQ | |
| | KQLTFFSLTPQYCHYKMIPPYILLATGLLQPSVPWR | |
| Monodelphis | MNSKTGPSVGDATLRRRIKPWEFVAFFNPQELRKETCLLYEIKWGNQNIW | 490 |
| domestica | RHSNQNTSQHAEINFMEKFTAERHFNSSVRCSITWFLSWSPCWECSKAIR | |
| APOBEC-1 | KFLDHYPNVTLAIFISRLYWHMDQQHRQGLKELVHSGVTIQIMSYSEYHY | |
| | CWRNFVDYPQGEEDYWPKYPYLWIMLYVLELHCIILGLPPCLKISGSHSN | |
| | QLALFSLDLQDCHYQKIPYNVLVATGLVQPFVTWR | |
| Pongo | MAQKEEAAAATEAASQNGEDLENLDDPEKLKELIELPPFEIVTGERLPAN | 491 |
| pygmaeus | FFKFQFRNVEYSSGRNKTFLCYVVEAQGKGGQVQASRGYLEDEHAAAHAE | |
| APOBEC-2 | EAFFNTILPAFDPALRYNVTWYVSSSPCAACADRIIKTLSKTKNLRLLIL | |
| | VGRLFMWEELEIQDALKKLKEAGCKLRIMKPQDFEYVWQNFVEQEEGESK | |
| | AFQPWEDIQENFLYYEEKLADILK | |
| Bos taurus | MAQKEEAAAAAEPASQNGEEVENLEDPEKLKELIELPPFEIVTGERLPAH | 492 |
| APOBEC-2 | YFKFQFRNVEYSSGRNKTFLCYVVEAQSKGGQVQASRGYLEDEHATNHAE | |
| | EAFFNSIMPTFDPALRYMVTWYVSSSPCAACADRIVKTLNKTKNLRLLIL | |
| | VGRLFMWEEPEIQAALRKLKEAGCRLRIMKPQDFEYIWQNFVEQEEGESK | |
| | AFEPWEDIQENFLYYEEKLADILK | |
| Mus musculus | MQPQRLGPRAGMGPFCLGCSHRKCYSPIRNLISQETFKFHFKNLGYAKGR | 493 |
| mAPOBEC-3 | KDTFLCYEVTRKDCDSPVSLHHGVFKNKDNIHAEICFLYWFHDKVLKVLS | |
| | PREEFKITWYMSWSPCFECAEQIVRFLATHHNLSLDIFSSRLYNVQDPET | |
| | QQNLCRLVQEGAQVAAMDLYEFKKCWKKFVDNGGRRFRPWKRLLINFRYQ | |
| | DSKLQEILRPCYISVPSSSSSTLSNICLTKGLPETRFWVEGRRMDPLSEE | |
| | EFYSQFYNQRVKHLCYYHRMKPYLCYQLEQFNGQAPLKGCLLSEKGKQHA | |
| | EILFLDKIRSMELSQVTITCYLTWSPCPNCAWQLAAFKRDRPDLILHIYT | |
| | SRLYFHWKRPFQKGLCSLWQSGILVDVMDLPQFTDCWTNFVNPKRPFWPW | |
| | KGLEIISRRTQRRLRRIKESWGLQDLVNDFGNLQLGPPMS | |
| Mouse | MGPFCLGCSHRKCYSPIRNLISQETFKFHFKNLGYAKGRKDTFLCYEVTR | 494 |
| APOBEC-3 | KDCDSPVSLHHGVFKNKDNIHAEICFLYWFHDKVLKVLSPREEFKITWYM | |
| | SWSPCFECAEQIVRFLATHHNLSLDIFSSRLYNVQDPETQQNLCRLVQEG | |
| | AQVAAMDLYEFKKCWKKFVDNGGRRFRPWKRLLINFRYQDSKLQEILRPC | |
| | YIPVPSSSSSTLSNICLTKGLPETRFCVEGRRMDPLSEEEFYSQFYNQRV | |
| | KHLCYYHRMKPYLCYQLEQFNGQAPLKGCLLSEKGKQHAEILFLDKIRSM | |
| | ELSQVTITCYLTWSPCPNCAWQLAAFKRDRPDLILHIYTSRLYFHWKRPF | |
| | QKGLCSLWQSGILVDVMDLPQFTDCWTNFVNPKRPFWPWKGLEIISRRTQ | |
| | RRLRRIKESWGLQDLVNDFGNLQLGPPMS | |
| Rat | MGPFCLGCSHRKCYSPIRNLISQETFKFHFKNRLRYAIDRKDTFLCYEVT | 495 |
| APOBEC-3 | RKDCDSPVSLHHGVFKNKDNIHAEICFLYWFHDKVLKVLSPREEFKITWY | |
| | MSWSPCFECAEQVLRFLATHHNLSLDIFSSRLYNIRDPENQQNLCRLVQE | |
| | GAQVAAMDLYEFKKCWKKFVDNGGRRFRPWKKLLTNFRYQDSKLQEILRP | |
| | CYIPVPSSSSSTLSNICLTKGLPETRFCVERRRVHLLSEEEFYSQFYNQR | |
| | VKHLCYYHGVKPYLCYQLEQFNGQAPLKGCLLSEKGKQHAEILFLDKIRS | |
| | MELSQVIITCYLTWSPCPNCAWQLAAFKRDRPDLILHIYTSRLYFHWKRP | |
| | FQKGLCSLWQSGILVDVMDLPQFTDCWTNFVNPKRPFWPWKGLEIISRRT | |
| | QRRLHRIKESWGLQDLVNDFGNLQLGPPMS | |
| Human | MEASPASGPRHLMDPHIFTSNFNNGIGRHKTYLCYEVERLDNGTSVKMDQ | 496 |
| APOBEC-3A | HRGFLHNQAKNLLCGFYGRHAELRFLDLVPSLQLDPAQIYRVTWFISWSP | |
| | CFSWGCAGEVRAFLQENTHVRLRIFAARIYDYDPLYKEALQMLRDAGAQV | |
| | SIMTYDEFKHCWDTFVDHQGCPFQPWDGLDEHSQALSGRLRAILQNQGN | |
| Human | MKPHFRNTVERMYRDTFSYNFYNRPILSRRNTVWLCYEVKTKGPSRPRLD | 497 |
| APOBEC-3F | AKIFRGQVYSQPEHHAEMCFLSWFCGNQLPAYKCFQITWFVSWTPCPDCV | |
| | AKLAEFLAEHPNVTLTISAARLYYYWERDYRRALCRLSQAGARVKIMDDE | |
| | EFAYCWENFVYSEGQPFMPWYKFDDNYAFLHRTLKEILRNPMEAMYPHIF | |
| | YFHFKNLRKAYGRNESWLCFTMEVVKHHSPVSWKRGVFRNQVDPETHCHA | |
| | ERCFLSWFCDDILSPNTNYEVTWYTSWSPCPECAGEVAEFLARHSNVNLT | |
| | IFTARLYYFWDTDYQEGLRSLSQEGASVEIMGYKDFKYCWENFVYNDDEP | |
| | FKPWKGLKYNFLFLDSKLQEILE | |
| Rhesus | MVEPMDPRTFVSNFNNRPILSGLNTVWLCCEVKTKDPSGPPLDAKIFQGK | 498 |
| macaque | VYSKAKYHPEMRFLRWFHKWRQLHHDQEYKVTWYVSWSPCTRCANSVATF | |
| APOBEC-3G | LAKDPKVTLTIFVARLYYFWKPDYQQALRILCQKRGGPHATMKIMNYNEF | |
| | QDCWNKFVDGRGKPFKPRNNLPKHYTLLQATLGELLRHLMDPGIFTSNEN | |
| | NKPWVSGQHETYLCYKVERLHNDTWVPLNQHRGFLRNQAPNIHGFPKGRH | |
| | AELCFLDLIPFWKLDGQQYRVTCFTSWSPCFSCAQEMAKFISNNEHVSLC | |
| | IFAARIYDDQGRYQEGLRALHRDGAKIAMMNYSEFEYCWDTFVDRQGRPF | |
| | QPWDGLDEHSQALSGRLRAI | |
| Chimpanzee | MKPHFRNPVERMYQDTFSDNFYNRPILSHRNTVWLCYEVKTKGPSRPPLD | 499 |
| APOBEC-3G | AKIFRGQVYSKLKYHPEMRFFHWFSKWRKLHRDQEYEVTWYISWSPCTKC | |
| | TRDVATFLAEDPKVTLTIFVARLYYFWDPDYQEALRSLCQKRDGPRATMK | |
| | IMNYDEFQHCWSKFVYSQRELFEPWNNLPKYYILLHIMLGEILRHSMDPP | |
| | TFTSNFNNELWVRGRHETYLCYEVERLHNDTWVLLNQRRGFLCNQAPHKH | |
| | GFLEGRHAELCFLDVIPFWKLDLHQDYRVTCFTSWSPCFSCAQEMAKFIS | |
| | NNKHVSLCIFAARIYDDQGRCQEGLRTLAKAGAKISIMTYSEFKHCWDTF | |
| | VDHQGCPFQPWDGLEEHSQALSGRLRAILQNQGN | |
| Green monkey | MNPQIRNMVEQMEPDIFVYYFNNRPILSGRNTVWLCYEVKTKDPSGPPLD | 500 |
| APOBEC-3G | ANIFQGKLYPEAKDHPEMKFLHWFRKWRQLHRDQEYEVTWYVSWSPCTRC | |
| | ANSVATFLAEDPKVTLTIFVARLYYFWKPDYQQALRILCQERGGPHATMK | |
| | IMNYNEFQHCWNEFVDGQGKPFKPRKNLPKHYTLLHATLGELLRHVMDPG | |
| | TFTSNFNNKPWVSGQRETYLCYKVERSHNDTWVLLNQHRGFLRNQAPDRH | |
| | GFPKGRHAELCFLDLIPFWKLDDQQYRVTCFTSWSPCFSCAQKMAKFISN | |
| | NKHVSLCIFAARIYDDQGRCQEGLRTLHRDGAKIAVMNYSEFEYCWDTFV | |
| | DRQGRPFQPWDGLDEHSQALSGRLRAI | |
| Human | MKPHFRNTVERMYRDTFSYNFYNRPILSRRNTVWLCYEVKTKGPSRPPLD | 501 |
| APOBEC-3G | AKIFRGQVYSELKYHPEMRFFHWFSKWRKLHRDQEYEVTWYISWSPCTKC | |
| | TRDMATFLAEDPKVTLTIFVARLYYFWDPDYQEALRSLCQKRDGPRATMK | |
| | IMNYDEFQHCWSKFVYSQRELFEPWNNLPKYYILLHIMLGEILRHSMDPP | |
| | TFTFNFNNEPWVRGRHETYLCYEVERMHNDTWVLLNQRRGFLCNQAPHKH | |
| | GFLEGRHAELCFLDVIPFWKLDLDQDYRVTCFTSWSPCFSCAQEMAKFIS | |
| | KNKHVSLCIFTARIYDDQGRCQEGLRTLAEAGAKISIMTYSEFKHCWDTF | |
| | VDHQGCPFQPWDGLDEHSQDLSGRLRAILQNQEN | |
| Human | MKPHFRNTVERMYRDTFSYNFYNRPILSRRNTVWLCYEVKTKGPSRPRLD | 502 |
| APOBEC-3F | AKIFRGQVYSQPEHHAEMCFLSWFCGNQLPAYKCFQITWFVSWTPCPDCV | |
| | AKLAEFLAEHPNVTLTISAARLYYYWERDYRRALCRLSQAGARVKIMDDE | |
| | EFAYCWENFVYSEGQPFMPWYKFDDNYAFLHRILKEILRNPMEAMYPHIF | |
| | YFHFKNLRKAYGRNESWLCFTMEVVKHHSPVSWKRGVFRNQVDPETHCHA | |
| | ERCFLSWFCDDILSPNTNYEVTWYTSWSPCPECAGEVAEFLARHSNVNLT | |
| | IFTARLYYFWDTDYQEGLRSLSQEGASVEIMGYKDFKYCWENFVYNDDEP | |
| | FKPWKGLKYNFLFLDSKLQEILE | |
| Human | MNPQIRNPMERMYRDTFYDNFENEPILYGRSYTWLCYEVKIKRGRSNLLW | 503 |
| APOBEC-3B | DTGVFRGQVYFKPQYHAEMCFLSWFCGNQLPAYKCFQITWFVSWTPCPDC | |
| | VAKLAEFLSEHPNVTLTISAARLYYYWERDYRRALCRLSQAGARVTIMDY | |
| | EEFAYCWENFVYNEGQQFMPWYKFDENYAFLHRTLKEILRYLMDPDTFTF | |
| | NFNNDPLVLRRRQTYLCYEVERLDNGTWVLMDQHMGFLCNEAKNLLCGFY | |
| | GRHAELRFLDLVPSLQLDPAQIYRVTWFISWSPCFSWGCAGEVRAFLQEN | |
| | THVRLRIFAARIYDYDPLYKEALQMLRDAGAQVSIMTYDEFEYCWDTFVY | |
| | RQGCPFQPWDGLEEHSQALSGRLRAILQNQGN | |
| Rat | MQPQGLGPNAGMGPVCLGCSHRRPYSPIRNPLKKLYQQTFYFHFKNVRYA | 504 |
| APOBEC-3B | WGRKNNFLCYEVNGMDCALPVPLRQGVFRKQGHIHAELCFIYWFHDKVLR | |
| | VLSPMEEFKVTWYMSWSPCSKCAEQVARFLAAHRNLSLAIFSSRLYYYLR | |
| | NPNYQQKLCRLIQEGVHVAAMDLPEFKKCWNKFVDNDGQPFRPWMRLRIN | |
| | FSFYDCKLQEIFSRMNLLREDVFYLQFNNSHRVKPVQNRYYRRKSYLCYQ | |
| | LERANGQEPLKGYLLYKKGEQHVEILFLEKMRSMELSQVRITCYLTWSPC | |
| | PNCARQLAAFKKDHPDLILRIYTSRLYFWRKKFQKGLCTLWRSGIHVDVM | |
| | DLPQFADCWTNFVNPQRPFRPWNELEKNSWRIQRRLRRIKESWGL | |
| Bovine | MDGWEVAFRSGTVLKAGVLGVSMTEGWAGSGHPGQGACVWTPGTRNTMNL | 505 |
| APOBEC-3B | LREVLFKQQFGNQPRVPAPYYRRKTYLCYQLKQRNDLTLDRGCFRNKKQR | |
| | HAERFIDKINSLDLNPSQSYKIICYITWSPCPNCANELVNFITRNNHLKL | |
| | EIFASRLYFHWIKSFKMGLQDLQNAGISVAVMTHTEFEDCWEQFVDNQSR | |
| | PFQPWDKLEQYSASIRRRLQRILTAPI | |
| Chimpanzee | MNPQIRNPMEWMYQRTFYYNFENEPILYGRSYTWLCYEVKIRRGHSNLLW | 506 |
| APOBEC-3B | DTGVFRGQMYSQPEHHAEMCFLSWFCGNQLSAYKCFQITWFVSWTPCPDC | |
| | VAKLAKFLAEHPNVTLTISAARLYYYWERDYRRALCRLSQAGARVKIMDD | |
| | EEFAYCWENFVYNEGQPFMPWYKFDDNYAFLHRTLKEIIRHLMDPDTFTF | |
| | NFNNDPLVLRRHQTYLCYEVERLDNGTWVLMDQHMGFLCNEAKNLLCGFY | |
| | GRHAELRFLDLVPSLQLDPAQIYRVTWFISWSPCFSWGCAGQVRAFLQEN | |
| | THVRLRIFAARIYDYDPLYKEALQMLRDAGAQVSIMTYDEFEYCWDTFVY | |
| | RQGCPFQPWDGLEEHSQALSGRLRAILQVRASSLCMVPHRPPPPPQSPGP | |
| | CLPLCSEPPLGSLLPTGRPAPSLPFLLTASFSFPPPASLPPLPSLSLSPG | |
| | HLPVPSFHSLTSCSIQPPCSSRIRETEGWASVSKEGRDLG | |
| Human | MNPQIRNPMKAMYPGTFYFQFKNLWEANDRNETWLCFTVEGIKRRSVVSW | 507 |
| APOBEC-3C | KTGVFRNQVDSETHCHAERCFLSWFCDDILSPNTKYQVTWYTSWSPCPDC | |
| | AGEVAEFLARHSNVNLTIFTARLYYFQYPCYQEGLRSLSQEGVAVEIMDY | |
| | EDFKYCWENFVYNDNEPFKPWKGLKTNFRLLKRRLRESLQ | |
| Gorilla | MNPQIRNPMKAMYPGTFYFQFKNLWEANDRNETWLCFTVEGIKRRSVVSW | 508 |
| APOBEC-3C | KTGVFRNQVDSETHCHAERCFLSWECDDILSPNTNYQVTWYTSWSPCPEC | |
| | AGEVAEFLARHSNVNLTIFTARLYYFQDTDYQEGLRSLSQEGVAVKIMDY | |
| | KDFKYCWENFVYNDDEPFKPWKGLKYNFRFLKRRLQEILE | |
| Human | MEASPASGPRHLMDPHIFTSNFNNGIGRHKTYLCYEVERLDNGTSVKMDQ | 509 |
| APOBEC-3A | HRGFLHNQAKNLLCGFYGRHAELRFLDLVPSLQLDPAQIYRVTWFISWSP | |
| | CFSWGCAGEVRAFLQENTHVRLRIFAARIYDYDPLYKEALQMLRDAGAQV | |
| | SIMTYDEFKHCWDTFVDHQGCPFQPWDGLDEHSQALSGRLRAILQNQGN | |
| Rhesus | MDGSPASRPRHLMDPNTFTFNFNNDLSVRGRHQTYLCYEVERLDNGTWVP | 510 |
| macaque | MDERRGFLCNKAKNVPCGDYGCHVELRFLCEVPSWQLDPAQTYRVTWFIS | |
| APOBEC-3A | WSPCFRRGCAGQVRVFLQENKHVRLRIFAARIYDYDPLYQEALRTLRDAG | |
| | AQVSIMTYEEFKHCWDTFVDRQGRPFQPWDGLDEHSQALSGRLRAILQNQ | |
| | GN | |
| Bovine | MDEYTFTENFNNQGWPSKTYLCYEMERLDGDATIPLDEYKGFVRNKGLDQ | 511 |
| APOBEC-3A | PEKPCHAELYFLGKIHSWNLDRNQHYRLTCFISWSPCYDCAQKLTTFLKE | |
| | NHHISLHILASRIYTHNRFGCHQSGLCELQAAGARITIMTFEDFKHCWET | |
| | FVDHKGKPFQPWEGLNVKSQALCTELQAILKTQQN | |
| Human | MALLTAETFRLQFNNKRRLRRPYYPRKALLCYQLTPQNGSTPTRGYFENK | 512 |
| APOBEC-3H | KKCHAEICFINEIKSMGLDETQCYQVTCYLTWSPCSSCAWELVDFIKAHD | |
| | HLNLGIFASRLYYHWCKPQQKGLRLLCGSQVPVEVMGFPKFADCWENFVD | |
| | HEKPLSFNPYKMLEELDKNSRAIKRRLERIKIPGVRAQGRYMDILCDAEV | |
| Rhesus | MALLTAKTFSLQFNNKRRVNKPYYPRKALLCYQLTPQNGSTPTRGHLKNK | 513 |
| macaque | KKDHAEIRFINKIKSMGLDETQCYQVTCYLTWSPCPSCAGELVDFIKAHR | |
| APOBEC-3H | HLNLRIFASRLYYHWRPNYQEGLLLLCGSQVPVEVMGLPEFTDCWENFVD | |
| | HKEPPSFNPSEKLEELDKNSQAIKRRLERIKSRSVDVLENGLRSLQLGPV | |
| | TPSSSIRNSR | |
| Human | MNPQIRNPMERMYRDTFYDNFENEPILYGRSYTWLCYEVKIKRGRSNLLW | 514 |
| APOBEC-3D | DTGVFRGPVLPKRQSNHRQEVYFRFENHAEMCFLSWFCGNRLPANRRFQI | |
| | TWFVSWNPCLPCVVKVTKFLAEHPNVTLTISAARLYYYRDRDWRWVLLRL | |
| | HKAGARVKIMDYEDFAYCWENFVCNEGQPFMPWYKFDDNYASLHRTLKEI | |
| | LRNPMEAMYPHIFYFHFKNLLKACGRNESWLCFTMEVTKHHSAVFRKRGV | |
| | FRNQVDPETHCHAERCFLSWFCDDILSPNTNYEVTWYTSWSPCPECAGEV | |
| | AEFLARHSNVNLTIFTARLCYFWDTDYQEGLCSLSQEGASVKIMGYKDFV | |
| | SCWKNFVYSDDEPFKPWKGLQTNFRLLKRRLREILQ | |
| Human | MTSEKGPSTGDPTLRRRIEPWEFDVFYDPRELRKEACLLYEIKWGMSRKI | 515 |
| APOBEC-1 | WRSSGKNTINHVEVNFIKKFTSERDFHPSMSCSITWFLSWSPCWECSQAI | |
| | REFLSRHPGVTLVIYVARLFWHMDQQNRQGLRDLVNSGVTIQIMRASEYY | |
| | HCWRNFVNYPPGDEAHWPQYPPLWMMLYALELHCIILSLPPCLKISRRWQ | |
| | NHLTFFRLHLQNCHYQTIPPHILLATGLIHPSVAWR | |
| Mouse | MSSETGPVAVDPTLRRRIEPHEFEVFFDPRELRKETCLLYEINWGGRHSV | 516 |
| APOBEC-1 | WRHTSQNTSNHVEVNFLEKFTTERYFRPNTRCSITWFLSWSPCGECSRAI | |
| | TEFLSRHPYVTLFIYIARLYHHTDQRNRQGLRDLISSGVTIQIMTEQEYC | |
| | YCWRNFVNYPPSNEAYWPRYPHLWVKLYVLELYCIILGLPPCLKILRRKQ | |
| | PQLTFFTITLQTCHYQRIPPHLLWATGLK | |
| Rat | MSSETGPVAVDPTLRRRIEPHEFEVFFDPRELRKETCLLYEINWGGRHSI | 517 |
| APOBEC-1 | WRHTSQNTNKHVEVNFIEKFTTERYFCPNTRCSITWFLSWSPCGECSRAI | |
| | TEFLSRYPHVTLFIYIARLYHHADPRNRQGLRDLISSGVTIQIMTEQESG | |
| | YCWRNFVNYSPSNEAHWPRYPHLWVRLYVLELYCIILGLPPCLNILRRKQ | |
| | PQLTFFTIALQSCHYQRLPPHILWATGLK | |
| Human | MAQKEEAAVATEAASQNGEDLENLDDPEKLKELIELPPFEIVTGERLPAN | 518 |
| APOBEC-2 | FFKFQFRNVEYSSGRNKTFLCYVVEAQGKGGQVQASRGYLEDEHAAAHAE | |
| | EAFFNTILPAFDPALRYNVTWYVSSSPCAACADRIIKTLSKTKNLRLLIL | |
| | VGRLFMWEEPEIQAALKKLKEAGCKLRIMKPQDFEYVWQNFVEQEEGESK | |
| | AFQPWEDIQENFLYYEEKLADILK | |
| Mouse | MAQKEEAAEAAAPASQNGDDLENLEDPEKLKELIDLPPFEIVTGVRLPVN | 519 |
| APOBEC-2 | FFKFQFRNVEYSSGRNKTFLCYVVEVQSKGGQAQATQGYLEDEHAGAHAE | |
| | EAFFNTILPAFDPALKYNVTWYVSSSPCAACADRILKTLSKTKNLRLLIL | |
| | VSRLFMWEEPEVQAALKKLKEAGCKLRIMKPQDFEYIWQNFVEQEEGESK | |
| | AFEPWEDIQENFLYYEEKLADILK | |
| Rat | MAQKEEAAEAAAPASQNGDDLENLEDPEKLKELIDLPPFEIVTGVRLPVN | 520 |
| APOBEC-2 | FFKFQFRNVEYSSGRNKTFLCYVVEAQSKGGQVQATQGYLEDEHAGAHAE | |
| | EAFFNTILPAFDPALKYNVTWYVSSSPCAACADRILKTLSKTKNLRLLIL | |
| | VSRLFMWEEPEVQAALKKLKEAGCKLRIMKPQDFEYLWQNFVEQEEGESK | |
| | AFEPWEDIQENFLYYEEKLADILK | |
| Bovine | MAQKEEAAAAAEPASQNGEEVENLEDPEKLKELIELPPFEIVTGERLPAH | 521 |
| APOBEC-2 | YFKFQFRNVEYSSGRNKTFLCYVVEAQSKGGQVQASRGYLEDEHATNHAE | |
| | EAFFNSIMPTFDPALRYMVTWYVSSSPCAACADRIVKTLNKTKNLRLLIL | |
| | VGRLFMWEEPEIQAALRKLKEAGCRLRIMKPQDFEYIWQNFVEQEEGESK | |
| | AFEPWEDIQENFLYYEEKLADILK | |
| Petromyzon | MTDAEYVRIHEKLDIYTFKKQFFNNKKSVSHRCYVLFELKRRGERRACFW | 522 |
| marinus | GYAVNKPQSGTERGIHAEIFSIRKVEEYLRDNPGQFTINWYSSWSPCADC | |
| CDA1 | AEKILEWYNQELRGNGHTLKIWACKLYYEKNARNQIGLWNLRDNGVGLNV | |
| | MVSEHYQCCRKIFIQSSHNQLNENRWLEKTLKRAEKRRSELSFMIQVKIL | |
| | HTTKSPAV | |
| Human | MKPHFRNTVERMYRDTFSYNFYNRPILSRRNTVWLCYEVKTKGPSRPPLD | 523 |
| APOBEC3G | AKIFRGQVYSELKYHPEMRFFHWFSKWRKLHRDQEYEVTWYISWSPCTKC | |
| D316R D317R | TRDMATFLAEDPKVTLTIFVARLYYFWDPDYQEALRSLCQKRDGPRATMK | |
| | FNYDEFQHCWSKFVYSQRELFEPWNNLPKYYILLHFMLGEILRHSMDPPT | |
| | FTFNENNEPWVRGRHETYLCYEVERMHNDTWVLLNQRRGFLCNQAPHKHG | |
| | FLEGRHAELCFLDVIPFWKLDLDQDYRVTCFTSWSPCFSCAQEMAKFISK | |
| | KHVSLCIFTARIYRRQGRCQEGLRTLAEAGAKISFTYSEFKHCWDTFVDH | |
| | QGCPFQPWDGLDEHSQDLSGRLRAILQNQEN | |
| Human | MDPPTFTFNFNNEPWWGRHETYLCYEVERMHNDTWVLLNQRRGFLCNQAP | 524 |
| APOBEC3G | HKHGFLEGRHAELCFLDVIPFWKLDLDQDYRVTCFTSWSPCFSCAQEMAK | |
| chain A | FISKNKHVSLCIFTARIYDDQGRCQEGLRTLAEAGAKISFTYSEFKHCWD | |
| | TFVDHQGCPFQPWDGLDEHSQDLSGRLRAILQ | |
| Human | MDPPTFTFNFNNEPWVRGRHETYLCYEVERMHNDTWVLLNQRRGFLCNQA | 525 |
| APOBEC3G | PHKHGFLEGRHAELCFLDVIPFWKLDLDQDYRVTCFTSWSPCFSCAQEMA | |
| chain A D120R | KFISKNKHVSLCIFTARIYRRQGRCQEGLRTLAEAGAKISFMTYSEFKHC | |
| D121R | WDTFVDHQGCPFQPWDGLDEHSQDLSGRLRAILQ | |
| Human | MEPIYEEYLANHGTIVKPYYWLSFSLDCSNCPYHIRTGEEARVSLTEFCQ | 526 |
| APOBEC-4 | IFGFPYGTTFPQTKHLTFYELKTSSGSLVQKGHASSCTGNYIHPESMLFE | |
| | MNGYLDSAIYNNDSIRHIILYSNNSPCNEANHCCISKMYNFLITYPGITL | |
| | SIYFSQLYHTEMDFPASAWNREALRSLASLWPRVVLSPISGGIWHSVLHS | |
| | FISGVSGSHVFQPILTGRALADRHNAYEINAITGVKPYFTDVLLQTKRNP | |
| | NTKAQEALESYPLNNAFPGQFFQMPSGQLQPNLPPDLRAPVVFVLVPLRD | |
| | LPPMHMGQNPNKPRNIVRHLNMPQMSFQETKDLGRLPTGRSVEIVEITEQ | |
| | FASSKEADEKKKKKGKK | |
| Mus musculus | MDSLLMKQKKFLYHFKNVRWAKGRHETYLCYVVKRRDSATSCSLDFGHLR | 527 |
| APOBEC-4 | NKSGCHVELLFLRYISDWDLDPGRCYRVTWFTSWSPCYDCARHVAEFLRW | |
| | NPNLSLRIFTARLYFCEDRKAEPEGLRRLHRAGVQIGIMTFKDYFYCWNT | |
| | FVENRERTFKAWEGLHENSVRLTRQLRRILLPLYEVDDLRDAFRMLGF | |
| Rattus | MEPLYEEYLTHSGTIVKPYYWLSVSLNCTNCPYHIRTGEEARVPYTEFHQ | 528 |
| norvegicus | TFGFPWSTYPQTKHLTFYELRSSSGNLIQKGLASNCTGSHTHPESMLFER | |
| APOBEC-4 | DGYLDSLIFHDSNIRHIILYSNNSPCDEANHCCISKMYNFLMNYPEVTLS | |
| | VFFSQLYHTENQFPTSAWNREALRGLASLWPQVTLSAISGGIWQSILETF | |
| | VSGISEGLTAVRPFTAGRTLTDRYNAYEINCITEVKPYFTDALHSWQKEN | |
| | QDQKVWAASENQPLHNTTPAQWQPDMSQDCRTPAVFMLVPYRDLPPIHVN | |
| | PSPQKPRTVVRHLNTLQLSASKVKALRKSPSGRPVKKEEARKGSTRSQEA | |
| | NETNKSKWKKQTLFIKSNICHLLEREQKKIGILSSWSV | |
| Macaca | MEPTYEEYLANHGTIVKPYYWLSFSLDCSNCPYHIRTGEEARVSLTEFCQ | 529 |
| fascicularis | IFGFPYGTTYPQTKHLTFYELKTSSGSLVQKGHASSCTGNYIHPESMLFE | |
| APOBEC-4 | MNGYLDSAIYNNDSIRHIILYCNNSPCNEANHCCISKVYNFLITYPGITL | |
| | SIYFSQLYHTEMDFPASAWNREALRSLASLWPRVVLSPISGGIWHSVLHS | |
| | FVSGVSGSHVFQPILTGRALTDRYNAYEINAITGVKPFFTDVLLHTKRNP | |
| | NTKAQMALESYPLNNAFPGQSFQMTSGIPPDLRAPVVFVLLPLRDLPPMH | |
| | MGQDPNKPRNIIRHLNMPQMSFQETKDLERLPTRRSVETVEITERFASSK | |
| | QAEEKTKKKKGKK | |
| Petromyzon | MAGYECVRVSEKLDFDTFEFQFENLHYATERHRTYVIFDVKPQSAGGRSR | 530 |
| marinus | RLWGYIINNPNVCHAELILMSMIDRHLESNPGVYAMTWYMSWSPCANCSS | |
| CDA-1 | KLNPWLKNLLEEQGHTLTMHFSRIYDRDREGDHRGLRGLKHVSNSFRMGV | |
| | VGRAEVKECLAEYVEASRRTLTWLDTTESMAAKMRRKLFCILVRCAGMRE | |
| | SGIPLHLFTLQTPLLSGRVVWWRV | |
| Petromyzon | MELREVVDCALASCVRHEPLSRVAFLRCFAAPSQKPRGTVILFYVEGAGR | 531 |
| marinus | GVTGGHAVNYNKQGTSIHAEVLLLSAVRAALLRRRRCEDGEEATRGCTLH | |
| CDA-2 | CYSTYSPCRDCVEYIQEFGASTGVRVVIHCCRLYELDVNRRRSEAEGVLR | |
| | SLSRLGRDFRLMGPRDAIALLLGGRLANTADGESGASGNAWVTETNVVEP | |
| | LVDMTGFGDEDLHAQVQRNKQIREAYANYASAVSLMLGELHVDPDKFPFL | |
| | AEFLAQTSVEPSGTPRETRGRPRGASSRGPEIGRQRPADFERALGAYGLF | |
| | LHPRIVSREADREEIKRDLIVVMRKHNYQGP | |
| Petromyzon | MAGDENVRVSEKLDFDTFEFQFENLHYATERHRTYVIFDVKPQSAGGRSR | 532 |
| marinus | RLWGYIINNPNVCHAELILMSMIDRHLESNPGVYAMTWYMSWSPCANCSS | |
| CDA-5 | KLNPWLKNLLEEQGHTLMMHFSRIYDRDREGDHRGLRGLKHVSNSFRMGV | |
| | VGRAEVKECLAEYVEASRRTLTWLDTTESMAAKMRRKLFCILVRCAGMRE | |
| | SGMPLHLFT | |
| Saccharomyces | MVTGGMASKWDQKGMDIAYEEAALGYKEGGVPIGGCLINNKDGSVLGRGH | 533 |
| cerevisiae | NMRFQKGSATLHGEISTLENCGRLEGKVYKDTTLYTTLSPCDMCTGAIIM | |
| CD | YGIPRCVVGENVNFKSKGEKYLQTRGHEVVVVDDERCKKIMKQFIDERPQ | |
| | DWFEDIGE | |
| Rat | MSSETGPVAVDPTLRRRIEPHEFEVFFDPRELRKETCLLYEINWGGRHSI | 534 |
| APOBEC-1 | WRHTSQNTNKHVEVNFIEKFTTERYFCPNTRCSITWFLSWSPCGECSRAI | |
| (delta 177-186) | TEFLSRYPHVTLFIYIARLYHHADPRNRQGLRDLISSGVTIQIMTEQESG | |
| | YCWRNFVNYSPSNEAHWPRYPHLWVRGLPPCLNILRRKQPQLTFFTIALQ | |
| | SCHYQRLPPHILWATGLK | |
| Rat | MSSETGPVAVDPTLRRRIEPHEFEVFFDPRELRKETCLLYEINWGGRHSI | 535 |
| APOBEC-1 | WRHTSQNTNKHVEVNFIEKFTTERYFCPNTRCSITWFLSWSPCGECSRAI | |
| (delta 202-213) | TEFLSRYPHVTLFIYIARLYHHADPRNRQGLRDLISSGVTIQIMTEQESG | |
| | YCWRNFVNYSPSNEAHWPRYPHLWVRLYVLELYCIILGLPPCLNILRRKQ | |
| | PQHYQRLPPHILWATGLK | |
| Mouse | MGPFCLGCSHRKCYSPIRNLISQETFKFHFKNLGYAKGRKDTFLCYEVTR | 536 |
| APOBEC-3 | KDCDSPVSLHHGVFKNKDNIHAEICFLYWFHDKVLKVLSPREEFKITWYM | |
| | SWSPCFECAEQIVRFLATHHNLSLDIFSSRLYNVQDPETQQNLCRLVQEG | |
| | AQVAAMDLYEFKKCWKKFVDNGGRRFRPWKRLLINFRYQDSKLQEILRPC | |
| | YIPVPSSSSSTLSNICLTKGLPETRFCVEGRRMDPLSEEEFYSQFYNQRV | |
| | KHLCYYHRMKPYLCYQLEQFNGQAPLKGCLLSEKGKQHAEILFLDKIRSM | |
| | ELSQVTITCYLTWSPCPNCAWQLAAFKRDRPDLILHIYTSRLYFHWKRPF | |
| | QKGLCSLWQSGILVDVMDLPQFTDCWTNFVNPKRPFWPWKGLEIISRRTQ | |
| | RRLRRIKESWGLQDLVNDFGNLQLGPPMS | |

In some embodiments, the amino acid sequence of the nucleobase editor (or the functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 3.

In some embodiments, the amino acid sequence of the nucleobase editor (or a functional fragment, functional variant, or domain thereof) comprises or consists of the amino acid sequence of a polypeptide set forth in Table 3, and further comprises 1 or more but less than 15% (less than 12%, less than 10%, less than 8%), amino acid variations (e.g., substitutions, additions, deletions, etc.). In some embodiments, the amino acid sequence of the nucleobase editor (or a functional fragment, functional variant, or domain thereof) comprises or consists of the amino acid sequence of a polypeptide set forth in Table 3, and further comprises or consists of at least about 1, 2, 3, 4, 5, 6, 7, 8, 9, or 10 amino acid variations (e.g., substitutions, additions, deletions, etc.). In some embodiments, the amino acid sequence of the nucleobase editor (or a functional fragment, functional variant, or domain thereof) comprises or consists of the amino acid sequence of a polypeptide set forth in Table 3, and further comprises or consists of about 1, 2, 3, 4, 5, 6, 7, 8, 9, or 10 amino acid variations (e.g., substitutions, additions, deletions, etc.). In some embodiments, the amino acid sequence of the nucleobase editor (or a functional fragment, functional variant, or domain thereof) comprises or consists of the amino acid sequence of a polypeptide set forth in Table 3, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the nucleobase editor (or a functional fragment, functional variant, or domain thereof) comprises or consists of the amino acid sequence of a polypeptide set forth in Table 3, and further comprises 1 or more but less than 15% (less than 12%, less than 10%, less than 8%), amino acid substitutions. In some embodiments, the amino acid sequence of the nucleobase editor (or a functional fragment, functional variant, or domain thereof) comprises or consists of the amino acid sequence of a polypeptide set forth in Table 3, and further comprises or consists of at least about 1, 2, 3, 4, 5, 6, 7, 8, 9, or 10 amino acid substitutions. In some embodiments, the amino acid sequence of the nucleobase editor (or a functional fragment, functional variant, or domain thereof) comprises or consists of the amino acid sequence of a polypeptide set forth in Table 3, and further comprises or consists of about 1, 2, 3, 4, 5, 6, 7, 8, 9, or 10 amino acid substitutions. In some embodiments, the amino acid sequence of the nucleobase editor (or a functional fragment, functional variant, or domain thereof) comprises or consists of the amino acid sequence of a polypeptide set forth in Table 3, and further comprises or consists of no more than about 1, 2, 3, 4, 5, 6, 7, 8, 9, or 10 amino acid substitutions.

In some embodiments, the amino acid sequence of nucleobase editor (or a functional fragment, functional variant, or domain thereof) comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 477-536.

In some embodiments, the amino acid sequence of the nucleobase editor (or a functional fragment, functional variant, or domain thereof) comprises or consists of the amino acid sequence of any one of SEQ ID NOS: 477-536, and further comprises 1 or more but less than 15% (less than 12%, less than 10%, less than 8%), amino acid variations (e.g., substitutions, additions, deletions, etc.). In some embodiments, the amino acid sequence of the nucleobase editor (or a functional fragment, functional variant, or domain thereof) comprises or consists of the amino acid sequence of any one of SEQ ID NOS: 477-536, and further comprises or consists of at least about 1, 2, 3, 4, 5, 6, 7, 8, 9, or 10 amino acid variations (e.g., substitutions, additions, deletions, etc.). In some embodiments, the amino acid sequence of the nucleobase editor (or a functional fragment, functional variant, or domain thereof) comprises or consists of the amino acid sequence of any one of SEQ ID NOS: 477-536, and further comprises or consists of about 1, 2, 3, 4, 5, 6, 7, 8, 9, or 10 amino acid variations (e.g., substitutions, additions, deletions, etc.). In some embodiments, the amino acid sequence of the nucleobase editor (or a functional fragment, functional variant, or domain thereof) comprises or consists of the amino acid sequence of any one of SEQ ID NOS: 477-536, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the nucleobase editor (or a functional fragment, functional variant, or domain thereof) comprises or consists of the amino acid sequence of any one of SEQ ID NOS: 477-536, and further comprises 1 or more but less than 15% (less than 12%, less than 10%, less than 8%), amino acid substitutions. In some embodiments, the amino acid sequence of the nucleobase editor (or a functional fragment, functional variant, or domain thereof) comprises or consists of the amino acid sequence of any one of SEQ ID NOS: 477-536, and further comprises or consists of at least about 1, 2, 3, 4, 5, 6, 7, 8, 9, or 10 amino acid substitutions. In some embodiments, the amino acid sequence of the nucleobase editor (or a functional fragment, functional variant, or domain thereof) comprises or consists of the amino acid sequence of any one of SEQ ID NOS: 477-536, and further comprises or consists of about 1, 2, 3, 4, 5, 6, 7, 8, 9, or 10 amino acid substitutions. In some embodiments, the amino acid sequence of the nucleobase editor (or the functional fragment or variant thereof) comprises or consists of the amino acid sequence of any one of SEQ ID NOS: 477-536, and further comprises or consists of no more than about 1, 2, 3, 4, 5, 6, 7, 8, 9, or 10 amino acid substitutions.

A nucleobase editor described herein can be further operably connected (e.g., fused) to another heterologous moiety (e.g., heterologous protein). In some embodiments, nucleobase editor described herein can be further operably connected (e.g., fused) to another heterologous moiety (e.g., heterologous protein). In some embodiments, the nucleobase editor is fused to an inhibitor of base excision repair, for example, a glycosylase inhibitor (UGI) domain or a nuclease dead inosine specific nuclease (dISN) domain.

### 4.3.2 Linkers

As described herein, a heterologous moiety (e.g., heterologous protein (e.g., reverse transcriptase, nucleobase editor)) can be directly operably connected or indirectly operably connected to a Cas endonuclease (e.g., described herein). In some embodiments, the heterologous protein is directly operably connected to a Cas endonuclease (e.g., described herein). In some embodiments, a heterologous polypeptide is directly operably connected to a Cas endonuclease (e.g., described herein) via a peptide bond. In some embodiments, a heterologous protein is indirectly operably connected to a Cas endonuclease (e.g., described herein). In some embodiments, a heterologous protein is indirectly operably connected to a Cas endonuclease (e.g., described herein) via a linker.

In some embodiments, a heterologous protein is indirectly operably connected to a Cas endonuclease (e.g., described herein) via a peptide linker. In some embodiments, a peptide linker is one or any combination of a cleavable linker, a non-cleavable linker, a flexible linker, a rigid linker, a helical linker, and/or a non-helical linker. In some embodiments, a peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, a linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker comprises or consists of glycine, serine, or both glycine and serine amino acid residues. In some embodiments, an amino acid sequence of the peptide linker comprises or consists of glycine, serine, and proline amino acid residues.

The amino acid sequence of exemplary peptide linkers is provided in Table 4.

TABLE 4
The Amino Acid Sequence of Exemplary Peptide Linker.

| | | SEQ ID |
|---|---|---|
| Description | Amino Acid Sequence | NO |
| A | GGG | 537 |
| B | GGGG | 538 |
| C | GGGGG | 539 |
| D | GGGGGG | 540 |
| E | GGGGGGG | 541 |
| F | GGGGGGGG | 542 |
| G | GSS | 543 |
| H | GSSGSS | 544 |
| I | GSSGSSGSS | 545 |
| J | GSSGSSGSSGSS | 546 |
| K | GSSGSSGSSGSSGSS | 547 |
| L | GSSGSSGSSGSSGSSGSS | 548 |
| M | GGS | 549 |
| N | GGSGGS | 550 |
| O | GGSGGSGGS | 551 |
| P | GGSGGSGGSGGS | 552 |
| Q | GGSGGSGGSGGSGGS | 553 |
| R | GGSGGSGGSGGSGGSGGS | 554 |
| S | GGGGS | 555 |
| T | GGGGSGGGGS | 556 |
| U | GGGGSGGGGSGGGGS | 557 |
| V | GGGGSGGGGSGGGGSGGGGSGGGGS | 558 |
| W | GGGGSGGGGSGGGGSGGGGSGGGGSGGGGS | 559 |
| X | GGSGGG | 560 |
| Y | GGGGGS | 561 |
| Z | GGSGSS | 562 |
| A-1 | GSSGGS | 563 |
| B-1 | GSS | 564 |
| C-1 | GSSGSS | 565 |
| D-1 | GSSGSSGSS | 566 |
| E-1 | GSSGSSGSSGSS | 567 |
| F-1 | GSSGSSGSSGSSGSS | 568 |
| G-1 | GSSGSSGSSGSSGSSGSS | 569 |
| H-1 | GGGGSS | 570 |
| I-1 | GGSGGGGSS | 571 |
| J-1 | GGSGSSGGG | 572 |
| K-1 | GGGGGSGSS | 573 |
| L-1 | GGGGSSGGS | 574 |
| M-1 | GSSGGSGGG | 575 |
| N-1 | GSSGGGGGS | 576 |
| O-1 | EAAAK | 577 |
| P-1 | EAAAKEAAAK | 578 |
| Q-1 | EAAAKEAAAKEAAAK | 579 |
| R-1 | EAAAKEAAAKEAAAKEAAAK | 580 |
| S-1 | EAAAKEAAAKEAAAKEAAAKEAAAK | 581 |
| T-1 | EAAAKEAAAKEAAAKEAAAKEAAAKEAAAK | 582 |
| U-1 | GGSGGGEAAAK | 583 |
| V-1 | GGSEAAAKGGG | 584 |
| W-1 | GGGGGSEAAAK | 585 |
| X-1 | GGGEAAAKGGS | 586 |
| Y-1 | EAAAKGGSGGG | 587 |
| Z-1 | EAAAKGGGGGS | 588 |
| A-2 | PAP | 589 |
| B-2 | PAPAP | 590 |
| C-2 | PAPAPAP | 591 |
| D-2 | PAPAPAPAP | 592 |
| E-2 | PAPAPAPAPAP | 593 |
| F-2 | PAPAPAPAPAPAP | 594 |
| G-2 | GGGPAP | 595 |
| H-2 | PAPGSS | 596 |
| I-2 | GGSGGGPAP | 597 |
| J-2 | GGSPAPGGG | 598 |
| K-2 | GGGGGSPAP | 599 |
| L-2 | GGGPAPGGS | 600 |
| M-2 | PAPGGSGGG | 601 |
| N-2 | PAPGGGGGS | 602 |
| O-2 | GGSGSSPAP | 603 |
| P-2 | GGSPAPGSS | 604 |
| Q-2 | GSSGGSPAP | 605 |
| R-2 | GSSPAPGGS | 606 |
| S-2 | PAPGGSGSS | 607 |
| T-2 | PAPGSSGGS | 608 |
| U-2 | GGGGSSPAP | 609 |
| V-2 | GGGPAPGSS | 610 |
| W-2 | GSSGGGPAP | 611 |
| X-2 | GSSPAPGGG | 612 |
| Y-2 | PAPGGGGSS | 613 |
| Z-2 | PAPGSSGGG | 614 |
| A-3 | GGSEAAAK | 615 |
| B-3 | PAPGGS | 616 |
| C-3 | GGGEAAAK | 617 |
| D-3 | EAAAKGGG | 618 |
| E-3 | GSSEAAAK | 619 |
| F-3 | EAAAKGSS | 620 |
| G-3 | EAAAKPAP | 621 |
| H-3 | PAPEAAAK | 622 |
| I-3 | GGSGSSEAAAK | 623 |
| J-3 | GGSEAAAKGSS | 624 |
| K-3 | GSSGGSEAAAK | 625 |
| L-3 | GSSEAAAKGGS | 626 |
| M-3 | EAAAKGGSGSS | 627 |
| N-3 | EAAAKGSSGGS | 628 |
| O-3 | GGSEAAAKPAP | 629 |
| P-3 | GGSPAPEAAAK | 630 |
| Q-3 | EAAAKGGSPAP | 631 |
| R-3 | EAAAKPAPGGS | 632 |
| S-3 | PAPGGSEAAAK | 633 |
| T-3 | PAPEAAAKGGS | 634 |
| U-3 | GGGGSSEAAAK | 635 |
| V-3 | GGGEAAAKGSS | 636 |
| W-3 | GSSGGGEAAAK | 637 |
| X-3 | GSSEAAAKGGG | 638 |
| Y-3 | EAAAKGGGGSS | 639 |
| Z-3 | EAAAKGSSGGG | 640 |
| A-4 | GGGEAAAKPAP | 641 |
| B-4 | GGGPAPEAAAK | 642 |
| C-4 | EAAAKGGGPAP | 643 |
| D-4 | EAAAKPAPGGG | 644 |
| E-4 | PAPGGGEAAAK | 645 |
| F-4 | PAPEAAAKGGG | 646 |
| G-4 | GSSEAAAKPAP | 647 |
| H-4 | GSSPAPEAAAK | 648 |
| I-4 | EAAAKGSSPAP | 649 |
| J-4 | EAAAKPAPGSS | 650 |
| K-4 | PAPGSSEAAAK | 651 |
| L-4 | PAPEAAAKGSS | 652 |
| M-4 | GGGGSEAAAKGGGGS | 653 |
| N-4 | EAAAKGGGGSEAAAK | 654 |
| O-4 | SGSETPGTSESATPES | 655 |
| P-4 | GSAGSAAGSGEF | 656 |
| Q-4 | SGGSSGGSSGSETPGTSESATPESSGGSSGGSS | 657 |
| R-4 | AEAAAKEAAAKEAAAKEAAAKALEAEAAAKEAAAKEAAAKE | 658 |
| | AAAKA | |

In some embodiments, an amino acid sequence of the peptide linker comprises or consists of the amino acid sequence of any one of the linkers set forth in Table 4. In some embodiments, the amino acid sequence of the peptide linker comprises or consists of the amino acid sequence of any one of the linkers set forth in Table 4, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of the linkers set forth in Table 4, comprising 1, 2, or 3 amino acid variations (e.g., substitutions, deletions, additions). In some embodiments, the amino acid sequence of the peptide linker comprises or consists of the amino acid sequence of any one of the linkers set forth in Table 4, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 4, comprising 1, 2, or 3 amino acid substitutions.

In some embodiments, an amino acid sequence of the peptide linker comprises or consists of the amino acid sequence of any one of SEQ ID NOS: 537-658. In some embodiments, the amino acid sequence of the peptide linker comprises or consists of the amino acid sequence of any one of SEQ ID NOS: 537-658, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of SEQ ID NOS: 537-658, comprising 1, 2, or 3 amino acid variations (e.g., substitutions, deletions, additions). In some embodiments, the amino acid sequence of the peptide linker comprises or consists of the amino acid sequence of any one of SEQ ID NOS: 537-658, and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 537-658, comprising 1, 2, or 3 amino acid substitutions.

In some embodiments, the linker is a linker (or a functional fragment, functional variant, or domain thereof) described in WO2021178720 or WO2023039424, the entire contents of which are incorporated herein by reference for all purposes.

### 4.3.3 Orientation

The heterologous moiety (or moieties) (e.g., heterologous protein(s)) and the Cas endonuclease (e.g., described herein) (or a functional fragment, functional variant, or domain thereof) can be arranged in any configuration or order as long as the Cas endonuclease protein (e.g., described herein) (or a functional fragment, functional variant, or domain thereof) maintains the ability to mediate its function and in the embodiments wherein the heterologous moiety (e.g., heterologous protein) has a specific function, the heterologous moiety (e.g., heterologous protein) can mediate its function.

In some embodiments, the heterologous moiety (e.g., heterologous protein) is operably connected to the N-terminus, C-terminus, or internally between the N-terminus and the C-terminus of the Cas endonuclease (or a functional fragment, functional variant, or domain thereof). In some embodiments, a heterologous moiety (e.g., heterologous protein) is operably connected to the C-terminus of the Cas endonuclease (or the functional fragment, functional variant, or domain thereof). In some embodiments, a heterologous moiety (e.g., heterologous protein) is operably connected to the N-terminus of the endonuclease (or the functional fragment, functional variant, or domain thereof) and a heterologous moiety (e.g., heterologous protein) is operably connected to the C-terminus of the endonuclease (or the functional fragment, functional variant, or domain thereof).

In some embodiments, the heterologous moiety is a heterologous protein (e.g., a polymerase (e.g., a reverse transcriptase), a nucleobase editor (e.g., a deaminase) (e.g., described herein)) forming a fusion protein with a Cas endonuclease (or a functional fragment, functional variant, or domain thereof) (e.g., described herein). In some embodiments, the fusion protein comprises from N- to C-terminus: a Cas endonuclease (or a functional fragment, functional variant, or domain thereof) (e.g., described herein) and a heterologous protein (e.g., a polymerase (e.g., a reverse transcriptase), a nucleobase editor (e.g., a deaminase) (e.g., described herein)). In some embodiments, the fusion protein comprises from N- to C-terminus: a Cas endonuclease (or a functional fragment, functional variant, or domain thereof) (e.g., described herein), a peptide linker (e.g., described herein), and a heterologous protein (e.g., a polymerase (e.g., a reverse transcriptase), a nucleobase editor (e.g., a deaminase) (e.g., described herein)). In this specific orientation, the C-terminus of the endonuclease (or the functional fragment, functional variant, or domain thereof) (e.g., described herein) is operably connected to the N-terminus of the heterologous (e.g., a polymerase (e.g., a reverse transcriptase), a nucleobase editor (e.g., a deaminase) (e.g., described herein)) either directly or indirectly through the peptide linker (e.g., described herein).

In some embodiments, the heterologous moiety is a heterologous protein (e.g., a polymerase (e.g., a reverse transcriptase), a nucleobase editor (e.g., a deaminase) (e.g., described herein)) forming a fusion protein with a Cas endonuclease (or a functional fragment, functional variant, or domain thereof) (e.g., described herein). In some embodiments, the fusion protein comprises from N- to C-terminus: a heterologous protein (e.g., a polymerase (e.g., a reverse transcriptase), a nucleobase editor (e.g., a deaminase) (e.g., described herein)) and a Cas endonuclease (or a functional fragment, functional variant, or domain thereof) (e.g., described herein). In some embodiments, the fusion protein comprises from N- to C-terminus: a heterologous protein (e.g., a polymerase (e.g., a reverse transcriptase), a nucleobase editor (e.g., a deaminase) (e.g., described herein)), a peptide linker (e.g., described herein), and a Cas endonuclease (or a functional fragment, functional variant, or domain thereof) (e.g., described herein). In this specific orientation, the C-terminus of the heterologous (e.g., a polymerase (e.g., a reverse transcriptase), a nucleobase editor (e.g., a deaminase) (e.g., described herein)) is operably connected to the N-terminus of the endonuclease (or the functional fragment, functional variant, or domain thereof) (e.g., described herein) either directly or indirectly through the peptide linker (e.g., described herein).

### 4.4 Methods of Making Proteins

Proteins described herein (e.g., Cas endonucleases, fusion proteins, and conjugates) may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the protein (e.g., the endonuclease, fusion protein, etc.). Such general methods are common knowledge in the art. The expression vector typically contains an expression cassette that includes nucleic acid sequences capable of bringing about expression of the nucleic acid molecule encoding the protein of interest (e.g., the Cas endonuclease, fusion protein, etc.), such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the protein of interest (e.g., the endonuclease, fusion protein, etc.). Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018). See also U.S. Pat. No. 5,762,939, the entire contents of each of which is incorporated by reference herein for all purposes. Proteins described herein (e.g., Cas endonucleases, fusion proteins, and protein conjugates) may be produced synthetically.

The disclosure provides, inter alia, methods of making a protein described herein (e.g., a Cas endonuclease (or a functional fragment, functional variant, or domain thereof), a fusion protein, etc.) comprising (a) introducing a nucleic acid molecule encoding the protein (e.g., the endonuclease (or the functional fragment, functional variant, or domain thereof), the fusion protein etc.) into a host cell; (b) culturing the host cell (e.g., under conditions and for a period of time sufficient to allow expression of the protein (e.g., the Cas endonuclease (or the functional fragment, functional variant, or domain thereof), the fusion protein etc.); and optionally isolating the protein (e.g., the Cas endonuclease (or the functional fragment, functional variant, or domain thereof), the fusion protein etc.) from the culture medium.

The disclosure further provides methods of making a protein described herein (e.g., a Cas endonuclease (or a functional fragment, functional variant, or domain thereof), a fusion protein etc.) comprising (a) recombinantly expressing the protein (e.g., the Cas endonuclease (or a functional fragment, functional variant, or domain thereof), the fusion protein etc.); (b) enriching, e.g., purifying, the protein (e.g., the Cas endonuclease (or a functional fragment, functional variant, or domain thereof), the fusion protein etc.); (c) evaluating the protein (e.g., the Cas endonuclease (or a functional fragment, functional variant, or domain thereof), the fusion protein etc.) for the presence of a process impurity or contaminant, and (d) formulating the protein (e.g., the Cas endonuclease (or a functional fragment, functional variant, or domain thereof), the fusion protein etc.) as a pharmaceutical composition if the protein (e.g., the Cas endonuclease (or a functional fragment, functional variant, or domain thereof), the fusion protein etc.) meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 4.5 Systems

Further provided herein are, inter alia, systems comprising a Cas endonuclease (or a functional fragment, functional variant, or domain thereof) (e.g., described herein) (or a fusion protein or conjugate of the any of the foregoing (e.g., described herein)), useful in, inter alia, editing a nucleic acid molecule (e.g., DNA, genome, gene (e.g., within a cell, e.g., within a cell in a subject (e.g., a mammalian subject, e.g., a human subject))) (e.g., in vivo, ex vivo, or in vitro). In some embodiments, the systems are useful in mediating the addition, deletion, or substitution of one or more nucleotides (e.g., nucleic acid (DNA) molecules) into/from a target nucleic acid (e.g., DNA) molecule (e.g., a target double stranded DNA molecule) (e.g., within a cell, e.g., within a cell in a subject (e.g., a mammalian subject, e.g., a human subject))).

As such, provided herein are systems comprising (a) (i) a Cas endonuclease described herein (or a functional fragment, functional variant, or domain thereof); (ii) a fusion protein comprising a Cas endonuclease described herein (or a functional fragment or functional variant thereof) (e.g., described herein); (iii) a conjugate comprising a Cas endonuclease described herein (or a functional fragment or functional variant thereof) (e.g., described herein); (iv) a nucleic acid molecule encoding (a)(i), (a)(ii), and/or (a)(iii) (e.g., a nucleic acid molecule described herein); (v) a vector comprising (a)(iv) (e.g., a vector described herein); (vi) a carrier comprising any one of (a)(i)-(a)(v) (e.g., a carrier described herein); or (vii) a composition comprising any one of (a)(i)-(a)(vi) (e.g., a pharmaceutical composition described herein).

In some embodiments, the system comprises (a) (i) a Cas endonuclease described herein (or a functional fragment, functional variant, or domain thereof); (ii) a fusion protein comprising a Cas endonuclease described herein (or a functional fragment or functional variant thereof) (e.g., described herein); (iii) a conjugate comprising a Cas endonuclease described herein (or a functional fragment or functional variant thereof) (e.g., described herein); (iv) a nucleic acid molecule encoding (a)(i), (a)(ii), or (a)(iii) (e.g., a nucleic acid molecule described herein); (v) a vector comprising (a)(iv) (e.g., a vector described herein); (vi) a carrier comprising any one of (a)(i)-(a)(v) (e.g., a carrier described herein); or (vii) a composition (e.g., a pharmaceutical composition) comprising any one of (a)(i)-(a)(vi) (e.g., a composition (e.g., a pharmaceutical composition) described herein); and (b) (i) first gRNA (e.g., a crRNA and a tracrRNA; a sgRNA; a template RNA (e.g., as described herein)) or (ii) a nucleic acid (e.g., DNA) molecule encoding the first gRNA (e.g., a crRNA and a tracrRNA; a sgRNA; template RNA (e.g., as described herein)).

As described above, the systems provided herein are useful in, inter alia, editing a nucleic acid molecule (e.g., DNA, genome, gene (e.g., within a cell, e.g., within a cell in a subject (e.g., a mammalian subject, e.g., a human subject))) (e.g., in vivo, ex vivo, or in vitro). In some embodiments, the systems provided herein may comprise one or more (e.g., any combination thereof or all) of the following features: (a) the Cas endonuclease (or the functional fragment, functional variant, or domain thereof) of the system is capable of binding a gRNA (e.g., described herein); (b) the Cas endonuclease (or a functional fragment, functional variant, or domain thereof) of the system is capable of forming a break in a target nucleic acid (e.g., DNA (e.g., dsDNA)) molecule (e.g., described herein); (c) the Cas endonuclease (or a functional fragment, functional variant, or domain thereof) of the system is capable of forming a single strand break in the edited strand (as defined herein) of a target double stranded nucleic acid (e.g., DNA (e.g., dsDNA)) molecule (e.g., described herein); (d) the Cas endonuclease (or a functional fragment, functional variant, or domain thereof) of the system is capable of forming a single strand break in a target double stranded nucleic acid (e.g., DNA (e.g., dsDNA)) molecule (e.g., described herein); (e) the Cas endonuclease (or a functional fragment, functional variant, or domain thereof) of the system is capable of forming a double strand break in a target double stranded nucleic acid (e.g., DNA (e.g., dsDNA)) molecule (e.g., described herein); (f) the Cas endonuclease (or a functional fragment, functional variant, or domain thereof) of the system is incapable of forming a double strand break in a target double stranded nucleic acid (e.g., DNA (e.g., dsDNA)) molecule (e.g., described herein); (g) the Cas endonuclease (or a functional fragment, functional variant, or domain thereof) of the system is capable of forming a single strand break in a target double stranded nucleic acid (e.g., DNA (e.g., dsDNA)) molecule (e.g., described herein) and is incapable of forming a double strand break in a target double stranded nucleic acid (e.g., DNA (e.g., dsDNA)) molecule (e.g., described herein) (e.g., exhibits nickase activity); (h) the Cas endonuclease (or a functional fragment, functional variant, or domain thereof) of the system is capable of forming a single strand break in the edited strand (as defined herein) of a target double stranded nucleic acid (e.g., DNA (e.g., dsDNA)) molecule (e.g., described herein) and is incapable of forming a double strand break in a target double stranded nucleic acid (e.g., DNA (e.g., dsDNA)) molecule (e.g., described herein); and/or (i) the system is capable of mediating the addition, deletion, or substitution of one or more nucleotides into/from a target nucleic acid (e.g., DNA) molecule (e.g., a target double stranded DNA molecule) (e.g., described herein).

### 4.5.1 Target Nucleic Acid Molecules

As described above, in some embodiments, the system is capable of mediating any one of the foregoing effects (see, e.g., § 4.5) in a target nucleic acid molecule. In some embodiments, the target nucleic acid molecule is a DNA molecule. In some embodiments, the target nucleic acid molecule is a dsDNA molecule. In some embodiments, a portion of the nucleotide sequence of the non-edited strand (as defined herein) of the target dsDNA molecule is complementary to at least a portion of the nucleotide sequence of a gRNA of the system (e.g., a gRNA described herein (see, e.g., § 4.5.2)).

In some embodiments, the target nucleic acid molecule is within the genome of cell (e.g., a eukaryotic cell) (e.g., within a subject (e.g., a human subject)). In some embodiments, the target nucleic acid molecule is a gene (e.g., within a cell (e.g., a eukaryotic cell) (e.g., within a subject (e.g., a human subject)). In some embodiments, the target nucleic acid molecule is within the genome of a cell (e.g., a eukaryotic cell) in vitro, ex vivo, or in vivo. In some embodiments, the target nucleic acid molecule is within the genome of a cell (e.g., a eukaryotic cell) within a subject (e.g., a human subject).

4.5.2 gRNAs

In some embodiments, the system comprises a guide RNA (gRNA). gRNAs are generally known in the art and described herein. See, e.g., Nishimasu et al. Cell 156, P935-949 (2014), the entire contents of which are incorporated herein by reference for all purposes. As described above, gRNAs include RNAs comprising a crRNA and a tracrRNA; sgRNAs; and template RNAs (e.g., as described herein). In some embodiments, the system comprises a nucleic acid (e.g., DNA) molecule encoding any one or more of the foregoing gRNAs (e.g., a crRNA and a tracrRNA; a sgRNA; a template RNA (e.g., as described herein)). Where gRNAs are described herein, the disclosure further covers a nucleic acid (e.g., DNA) molecule encoding the gRNA.

In some embodiments, at least a portion of the nucleotide sequence of the gRNA is complementary to a portion of the nucleotide sequence of the target nucleic acid molecule (e.g., described herein). In some embodiments, at least a portion of the nucleotide sequence of the gRNA is complementary to a portion of the nucleotide sequence of the non-edited strand (as defined herein) of a double stranded nucleic acid (e.g., dsDNA) target nucleic acid molecule (e.g., described herein). In some embodiments, at least a portion of the nucleotide sequence of the gRNA binds to a portion of the nucleotide sequence of the edited strand (as defined herein) of a double stranded nucleic acid (e.g., dsDNA) target nucleic acid molecule (e.g., described herein).

In some embodiments, the system comprises a crRNA and a tracrRNA (or a plurality of different crRNAs and a plurality of different tracrRNAs), wherein the crRNA and the tracrRNA are on separate RNA molecules. In some embodiments, the system comprises a nucleic acid molecule encoding a crRNA and a separate nucleic acid molecule encoding a tracrRNA. In some embodiments, the system comprises a plurality of nucleic acid molecules each encoding a different crRNA; and a plurality of nucleic acid molecules each encoding a tracrRNA (wherein each encoded tracrRNA can be the same or different).

In some embodiments, the system comprises a sgRNA (or a plurality of different sgRNAs). In some embodiments, the system comprises a nucleic acid (e.g., DNA) molecule encoding a sgRNA. In some embodiments, the system comprises a plurality of nucleic acid molecules, each encoding a different sgRNA. In some embodiments, the crRNA of each of the sgRNAs of the plurality is different. In some embodiments, the tracrRNA of each of the sgRNAs of the plurality is different. In some embodiments, the tracrRNA of each of the sgRNAs of the plurality is the same. In some embodiments the crRNA of each of the sgRNAs of the plurality is different and the tracrRNA of each of the sgRNAs of the plurality is the same.

In some embodiments, the system comprises a template RNA (e.g., a single template RNA, a plurality of different template RNAs) or a nucleic acid (e.g., DNA) molecule encoding the template RNA (or a plurality of nucleic acid (e.g., DNA) molecules each encoding a different template RNA). In some embodiments, the template RNA comprises from 5′ to 3′ a crRNA, a tracrRNA, a heterologous object sequence, and a 3′ target homology domain. In some embodiments, the template RNA further comprises a sequence that binds a polymerase (e.g., a reverse transcriptase, e.g., of a fusion protein described herein). In some embodiments, the template RNA comprises a crRNA, a tracrRNA, a sequence that binds a polymerase (e.g., a reverse transcriptase, e.g., of a fusion protein described herein), a heterologous object sequence, and a 3′ target homology domain. In some embodiments, the template RNA comprises from 5′ to 3′ a crRNA, a tracrRNA, a sequence that binds a polymerase (e.g., a reverse transcriptase, e.g., of a fusion protein described herein), a heterologous object sequence, and a 3′ target homology domain.

In some embodiments, the gRNA (e.g., the template RNA) comprises a nucleic acid molecule comprising a toe-loop, hairpin, stem-loop, pseudoknot (e.g., a Mpknot1 moiety), aptamer, G-quadraplex, tRNA, riboswitch, or ribozyme. In some embodiments, the gRNA (e.g., the template RNA) comprises a nucleic acid molecule comprising a pseudoknot (e.g., a Mpknot1 moiety). In some embodiments, the gRNA one or more 3′hairpin elements may be removed, e.g., as described in WO2018106727, the entire contents of which is incorporated herein by reference for all purposes. In some embodiments, a gRNA may contain additional hairpin structures, e.g., as described in Kocak et al. Nat Biotechnol 37(6):657-666 (2019), the entire contents of which is incorporated herein by reference for all purposes. Secondary structures (e.g., hairpins) in a gRNA can be predicted in silico by software tools, e.g., the RNAstructure tool available at ma.urmc.rochester.edu/RNAstructureWeb (Bellaousov et al. Nucleic Acids Res 41: W471-W474 (2013); incorporated by reference herein in its entirety).

Custom gRNA generators and algorithms are available commercially for use in the design of gRNAs.

4.5.2.1 Multiple gRNAs

In some embodiments, the system comprises a plurality of gRNAs (e.g., a plurality of sgRNAs, a plurality of template RNAs). In some embodiments, the system comprises a plurality of nucleic acid molecules each encoding a gRNA (e.g., a sgRNA, a template RNA).

In some embodiments, the system comprises a first gRNA (e.g., a sgRNA, a template RNA) and a second gRNA (e.g., a sgRNA, a template RNA). In some embodiments, the first gRNA is a sgRNA and the second gRNA is a sgRNA. In some embodiments, the first gRNA is a sgRNA and the second gRNA is a sgRNA, wherein the nucleotide sequence of the crRNA of the first and second gRNAs is different. In some embodiments, the first gRNA is a template RNA and the second gRNA is a sgRNA. In some embodiments, the first gRNA is a template RNA and the second gRNA is a sgRNA, wherein the nucleotide sequence of the crRNA of the first and second gRNAs is different.

In some embodiments, the second gRNA (e.g., sgRNA) is capable of directing the endonuclease (e.g., described herein) of the system to form a single strand break in the non-edited strand of a target double stranded nucleic acid (e.g., dsDNA) molecule. In some embodiments, at least a portion of the nucleotide sequence of the second gRNA (e.g., sgRNA) is complementary to a portion of the nucleotide sequence of the edited strand (as defined herein) of a double stranded nucleic acid (e.g., dsDNA) molecule. In some embodiments, at least a portion of the nucleotide sequence of the second gRNA (e.g., sgRNA) binds to a portion of the nucleotide sequence of the edited strand (as defined herein) of a double stranded nucleic acid (e.g., dsDNA) molecule.

In some embodiments, the second gRNA (e.g., sgRNA) is present on the same nucleic acid molecule as the first gRNA (or the nucleic acid (e.g., DNA) molecule encoding the second gRNA is present on the same nucleic acid (e.g., DNA) molecule encoding the first gRNA). In some embodiments, the second gRNA (e.g., sgRNA) is present on a different nucleic acid molecule as the first gRNA (or the nucleic acid (e.g., DNA) molecule encoding the second gRNA is present on a different nucleic acid (e.g., DNA) molecule encoding the first gRNA).

4.5.2.2 Modified gRNAs

In some embodiments, a gRNA (e.g., of a system described herein) comprises one or more modified nucleotide(s) (as defined herein) (referred to as a modified gRNA). The modified gRNA may have one or more different (e.g., improved) properties relative to a corresponding unmodified gRNA (e.g., one or more improved properties in vivo). For example, in some embodiments, the modified gRNA (e.g., an end-modified gRNA) may exhibit increased stability in a cell (e.g., ex vivo, in vivo, in vitro) (e.g., relative to an unmodified gRNA). In some embodiments, the modified gRNA (e.g., an end-modified gRNA) may exhibit increased stability in vivo (e.g., relative to an unmodified gRNA). In some embodiments, a system described herein utilizing a modified gRNA exhibits increased nucleic acid (e.g., gene) editing efficiency (e.g., relative to system comprising an unmodified gRNA). In some embodiments, a system described herein utilizing a modified gRNA exhibits increased on target nucleic acid (e.g., gene) editing (e.g., relative to system comprising an unmodified gRNA). In some embodiments, a system described herein utilizing a modified gRNA exhibits decreased off target nucleic acid (e.g., gene) editing (e.g., relative to system comprising an unmodified gRNA). In some embodiments, a system described herein utilizing a modified gRNA exhibits increased affinity for DNA molecules (e.g., a gRNA of the system exhibits increased affinity for DNA molecules) editing (e.g., relative to system comprising an unmodified gRNA).

Methods known in the art can be utilized to select and test modified gRNAs. For example, structure-guided and systematic approaches (e.g., as described in Mir, A., Alterman, J. F., Hassler, M. R. et al. Heavily and fully modified RNAs guide efficient SpyCas9-mediated genome editing. Nat Commun 9, 2641 (2018). https://doi.org/10.1038/s41467-018-05073-z; the entire contents of which is incorporated herein by reference for all purposes) can be employed to find and select modifications for gRNAs.

gRNA modifications are known in the art and described herein. See, e.g., Allen Daniel, et al, Using Synthetically Engineered Guide RNAs to Enhance CRISPR Genome Editing Systems in Mammalian Cells, Frontiers in Genome Editing, Vol 2 (article 617910) (2021) DOI=10.3389/fgeed.2020.617910; and Hendel A, Bak R O, Clark J T, et al. Chemically modified guide RNAs enhance CRISPR-Cas genome editing in human primary cells. Nat Biotechnol. 2015; 33(9):985-989. doi:10.1038/nbt.3290; the entire contents of each of which are incorporated herein by reference for all purposes.

The exemplary modifications provided herein are mainly described in reference to a gRNA. It is to be understood that corresponding modifications could be made to a DNA molecule encoding a gRNA. Such corresponding DNA modifications are known in the art and readily determined by a person of ordinary skill in the art. As such, modifications made to a “gRNA” also include corresponding modifications made to a DNA molecule encoding the gRNA.

### (i) Nature of the Modifications

Nucleotide modifications can include modification to any one of more of the nucleoside and/or the internucleoside linkage. Nucleoside modifications include modification to the sugar (e.g., ribose) moiety and/or the nucleobase. In some embodiments, the modified gRNA comprises one or more nucleotides comprising a modified sugar (e.g., ribose) moiety. In some embodiments, the modified gRNA comprises one or more nucleotides comprising a modified nucleobase. In some embodiments, the modified gRNA comprises one or more nucleotides comprising a modified internucleoside linkage. In some embodiments, the modified gRNA comprises one or more nucleotides comprising one, two, or three of a modified sugar (e.g., ribose) moiety, a modified nucleobase, and/or a modified internucleoside linkage. In some embodiments, the modified gRNA comprises one or more nucleotides comprising a modified sugar (e.g., ribose) moiety and a modified internucleoside linkage.

Exemplary nucleoside modifications are described below and also known in the art, see, e.g., WO2018107028A1 (see, e.g., Table 4 (as identified therein by a SEQ ID NO)); US20190316121; Hendel A, Bak R O, Clark J T, et al. Chemically modified guide RNAs enhance CRISPR-Cas genome editing in human primary cells. Nat Biotechnol. 33(9):985-989 (2015) doi:10.1038/nbt.3290; Mir et al. Nat Commun 9:2641 (2018) (see, e.g., supplementary Table 1); Allen D, Rosenberg M and Hendel A (2021) Using Synthetically Engineered Guide RNAs to Enhance CRISPR Genome Editing Systems in Mammalian Cells. Front. Genome Ed. 2:617910. doi: 10.3389/fgeed.2020.617910; the entire contents of each of which are incorporated herein by reference for all purposes, the entire contents of each of which is incorporated by reference herein for all purposes.

### (a) Sugar Modifications

In some embodiments, the modified gRNA comprises one or more nucleosides comprising a modified sugar (e.g., ribose) moiety.

The modified ribose moiety can comprise, for example, a substituent at any one or more position of the sugar (e.g., ribose), including e.g., positions 2′, 4′, and/or 5′. In some embodiments, the modified sugar (e.g., ribose) comprises a substituent at 2′ position of the sugar (e.g., ribose). In some embodiments, the modified sugar (e.g., ribose) comprises a substituent at 4′ position of the sugar (e.g., ribose). In some embodiments, the modified sugar (e.g., ribose) comprises a substituent at 5′ position of the sugar (e.g., ribose).

In some embodiments, the gRNA comprises any one or more of the following substituents (e.g., at any position of the sugar (e.g., ribose) (e.g., at position 2′)): a group for improving the stability of the gRNA, a group for improving the pharmacokinetic properties of the gRNA, a group for improving the pharmacodynamic properties of the gRNA, an RNA cleaving group, a reporter group, an intercalator, or other substituents having similar properties.

Exemplary substituents include, for example, but are not limited to, substitution (e.g., at any position of the sugar (e.g., ribose) (e.g., at position 2′)) with any one of the following: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀alkyl or C₂ to C₁₀ alkenyl and alkynyl. Additional exemplary substitutions (e.g., at any position of the sugar (e.g., ribose) (e.g., at position 2′)) include, for example, but are not limited to, substitution with any one of the following: O[(CH₂)ₙO]m, CH₃, O(CH₂)ₙOCH₃, O(CH₂)ₙNH₂, O(CH₂)ₙCH₃, O(CH₂)ₙONH₂, and O(CH₂)ₙON[(CH₂)·CH₃)]₂, where n and m are from 1 to about 10.

In some embodiments, the modified ribose comprises any one or more of the following modifications: 2′-O-methyl (2′-OMe); 2′0-methoxyethyl (2′-O-MOE); 2′deoxy-2′-fluoro (2′-F); 2′-arabino-fluoro (2′-Ara-F); 2′-O-benzyl; 2′-O-methyl-4-pyridine (2-O-methyl-4-pyridine (2′-O—CH₂Py(4)); 2′F-4′-Cα-OMe; or 2′,4′-di-Cα-OMe.

In some embodiments, the gRNA comprises any of the following substituents at the 2′-position of the sugar (e.g., ribose): C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, or a substituted silyl. In some embodiments, the gRNA comprises a 2′-methoxyethoxy (2′-O—CH₂CH₂OCH₃, also known as 2′-O-(2-methoxyethyl) or 2′-MOE) (see, e.g., Martin et al., Helv. Chim. Acta, 1995, 78:486-504, the entire contents of which is incorporated by reference herein for all purposes) (i.e., an alkoxy-alkoxy group). In some embodiments, the gRNA comprises a 2′-dimethylaminooxyethoxy, i.e., a O(CH₂)₂ON(CH₃)₂ group, also known as 2′-DMAOE; a 2′-dimethylaminoethoxyethoxy (also known in the art as 2′-O-dimethylaminoethoxyethyl or 2′-DMAEOE), i.e., 2′-O—CH₂—O—CH₂—N(CH₃)₂; a 5′-Me-2′-F nucleotide, a 5′-Me-2′-OMe nucleotide, a 5′-Me-2′-deoxynucleotide, (both R and S isomers in these three families); a 2′-alkoxyalkyl; and 2′-NMA (N-methylacetamide).

### Non-Bicyclic Sugar Modifications

In some embodiments, the modified sugar (e.g., ribose) moiety comprises a non-bicyclic modified sugar (e.g., ribose) moiety. In some embodiments, the modified sugar (e.g., ribose) moiety comprises a furanosyl ring comprising one or more substituent groups none of which bridges two atoms of the furanosyl ring to form a bicyclic structure. In some embodiments one or more non-bridging substituent of a non-bicyclic modified ribose moiety is branched. Such non bridging substituents may be at any position of the furanosyl, including but not limited to substituents at the 2′, 4′, and/or 5′ positions.

In some embodiments, non-bicyclic modified sugar (e.g., ribose) moiety comprises a substituent group at the 2′-position of the sugar (e.g., ribose). Examples of 2′-substituent groups suitable for non-bicyclic modified ribose moieties include but are not limited to: 2′-O-methyl (2′-OMe), 2′0-methoxyethyl (2′-O-MOE), 2′deoxy-2′-fluoro (2′-F), 2′-arabino-fluoro (2′-Ara-F), 2′-O-benzyl, 2′-O-methyl-4-pyridine (2-O-methyl-4-pyridine (2′-O—CH₂Py(4)), and 2′-O—N-alkyl acetamide (e.g., 2′-O—N-methyl acetamide (“NMA”), 2′-O—N-dimethyl acetamide, 2′-O—N-ethyl acetamide, and 2′-O—N-propyl acetamide). For example, see, e.g., U.S. Pat. No. 6,147,200, Prakash et al., 2003, Org. Lett., 5, 403-6, the entire contents of which is incorporated by reference herein for all purposes.

In some embodiments, the 2′-substituent group is a halo, allyl, amino, azido, SH, CN, OCN, CF₃, OCF₃, O—C₁-C₁₀alkoxy, O—C₁-C₁₀ substituted alkoxy, O—C₁-C₁₀alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O— alkyl, alkynyl, alkaryl, aralkyl, O-alkaryl, O-aralkyl, O(CH2)2SCH3,0(CH2)2ON(Rm)(Rn) or OCH2C(═O)— N(Rm)(Rn), where each Rₘ and Rₙ is, independently, H, an amino protecting group, or substituted or unsubstituted C₁-C₁₀ alkyl, or a 2′-substituent group described in any one of the following: Cook et al., U.S. Pat. No. 6,531,584; Cook et al., U.S. Pat. No. 5,859,221; and Cook et al., U.S. Pat. No. 6,005,087, the entire contents of which are incorporated herein by reference for all purposes. In some embodiments, these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In some embodiments, a 2′-substituted non-bicyclic modified nucleoside comprises a sugar (e.g., ribose) moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl. In some embodiments, a 2′-substituted non-bicyclic modified nucleoside comprises a sugar (e.g., ribose) moiety comprising a non-bridging 2′-substituent group selected from: F, OCF, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”). In some embodiments, a 2′-substituted non-bicyclic modified nucleoside comprises a sugar (e.g., ribose) moiety comprising a non-bridging 2′-substituent group selected from: F, OCH₃, OCH₂CH₂OCH₃, and OCH₂C(═O)—N(H)CH₃.

In some embodiments, non-bicyclic modified sugar (e.g., ribose) moiety comprises a substituent group at the 3′-position of the sugar (e.g., ribose). Examples of substituent groups suitable for the 3′-position of modified sugar (e.g., ribose) moieties include but are not limited to alkoxy (e.g., methoxy), alkyl (e.g., methyl, ethyl).

In some embodiments, non-bicyclic modified sugar (e.g., ribose) moiety comprises a substituent group at the 4′-position of the sugar (e.g., ribose). Examples of 4′-substituent groups suitable for non-bicyclic modified sugar (e.g., ribose) moieties include but are not limited to alkoxy (e.g., methoxy), alkyl, and those described in Manoharan et al., WO 2015/106128.

In some embodiments, non-bicyclic modified sugar (e.g., ribose) moiety comprises a substituent group at the 5′-position of the sugar (e.g., ribose). Examples of substituent groups suitable for the 5′-position of modified sugar (e.g., ribose) moieties include, but are not limited to, vinyl (e.g., 5′-vinyl), alkoxy (e.g., methoxy (e.g., 5′-methoxy)), and alkyl (e.g., methyl (R or S) (e.g., 5′-methyl (R or S)), ethyl).

In some embodiments, non-bicyclic modified sugar (e.g., ribose) moieties comprise more than one non-bridging sugar substituent, for example, 2′-F-5′-methyl sugar (e.g., ribose) moieties and the modified sugar (e.g., ribose) moieties and modified nucleosides described in Migawa et al., WO 2008/101157 and Rajeev et al., US2013/0203836, the entire contents of each of which is incorporated herein by reference for all purposes.

In some embodiments, modified furanosyl sugar (e.g., ribose) moieties and nucleosides incorporating such modified furanosyl sugar (e.g., ribose) moieties are further defined by isomeric configuration. For example, a 2′-deoxyfuranosyl sugar (e.g., ribose) moiety may be in seven isomeric configurations other than the naturally occurring β-D-deoxyribosyl configuration. Such modified sugar (e.g., ribose) moieties are described in, e.g., WO 2019/157531, the entire contents of which are incorporated by reference herein for all purposes.

In some embodiments, the sugar (e.g., ribose) modification comprises an unlocked nucleotide (UNA). UNA is unlocked acyclic nucleic acid, wherein any of the bonds of the sugar has been removed, forming an unlocked sugar (e.g., ribose) residue. For example, in some embodiments, the bonds between C1′-C4′ have been removed (i.e., the covalent carbon-oxygen-carbon bond between the C1′ and C4′ carbons). In some embodiments, the C2′-C3′ bond (i.e., the covalent carbon-carbon bond between the C2′ and C3′ carbons) of the sugar (e.g., ribose) have been removed. See, e.g., Nuc. Acids Symp. Series, 52, 133-134 (2008) and Fluiter et al., Mol. Biosyst., 2009, 10, 1039, the entire contents of which are incorporated herein by reference. UNAs and methods of making are known in the art. See, e.g., U.S. Pat. No. 8,314,227; and US2013/0096289; US2013/0011922; and US2011/0313020, the entire contents of each of which are hereby incorporated herein by reference.

### Bicyclic Sugar Modifications

In some embodiments, the modified sugar (e.g., ribose) moiety comprises a substituent that bridges two atoms of the furanosyl ring to form a second ring, resulting in a bicyclic sugar (e.g., ribose) moiety. In some embodiments, the bicyclic sugar (e.g., ribose) moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O—₂′ (“LNA”), 4′-CH₂—S-2′, 4′-(CH₂)2-O-2′ (“ENA”), 4′-CH(CH₃)—O-2′ (referred to as “constrained ethyl” or “cEt”), 4′-CH₂— O—CH₂-2′, 4′-CH₂—N(R)-2′, 4′-CH(CH₂OCH₃)—O-2′(“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′(see, e.g., Zhou, et al., J. Org. Chem., 2QQ9, 74, 118-134), 4′-CH₂—C(═CH₂)-2′ and analogs thereof (see, e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672). The entire contents of all of the foregoing references is incorporated by reference herein for all purposes.

In some embodiments, such 4′ to 2′ bridges independently comprise from 1 to 4 linked groups independently selected from: —[C(Rₐ)(R_{b})]n-, —[C(Rₐ)(R_{b})]n-O—, —C(Rₐ)═C(R_{b})—, —C(Rₐ)═N—, —C(═NRₐ)—, —C(═O)—, —C(═S)—, —O—, —Si(Rₐ)₂—, —S(═O)X—, and —N(Rₐ)—; wherein: x is 0, 1, or 2; n is 1, 2, 3, or 4; each Rₐ and Rb is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ1, NJ1J2, SJ1, N3, COOJ1, acyl (C(═O)—H), substituted acyl, CN, sulfonyl (S(=0)2-J1), or sulfoxyl (S(═O)-J1); and each J1 and J2 is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et. al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727. The entire contents of all of the foregoing references is incorporated by reference herein for all purposes.

In some embodiments, the modified sugar (e.g., ribose) comprises a constrained ethyl nucleotide comprising a 4′-CH(CH₃)—O-2′ bridge. In some embodiments, the constrained ethyl nucleotide is in the S conformation (S-cEt). In some embodiments, the modified sugar (e.g., ribose) comprises a conformationally restricted nucleotide (CRN). CRNs are nucleotide analogs with a linker connecting the C2′ and C4′ carbons of ribose or the C3 and C5′ carbons of ribose. Representative publications that teach the preparation of certain of the above include, but are not limited to, US2013/0190383; and WO2013/036868, the entire contents of each of which are hereby incorporated herein by reference.

In some embodiments, bicyclic sugar moieties and nucleosides incorporating such bicyclic sugar moieties are further defined by isomeric configuration. For example, an LNA nucleoside (described herein) may be in the α-L configuration or in the 3-D configuration. Herein, general descriptions of bicyclic nucleosides include both isomeric configurations. Any of the foregoing bicyclic nucleosides can be prepared having one or more stereochemical sugar configurations including for example α-L-ribofuranose and β-D-ribofuranose (see, e.g., WO 99/14226, the entire contents of which are incorporated herein by reference for all purposes).

Additional representative U.S. patents and U.S. patenttent Publications that teach the preparation of bicyclic nucleosides (e.g., locked nucleic acid) include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

### (b) Nucleobase Modifications

In some embodiments, the modified gRNA comprises one or more nucleotides comprising a modified nucleobase.

As used herein, “unmodified” nucleobases refer to the purine bases adenine (A) and guanine (G), and the pyrimidine bases thymine (T), cytosine (C), and uracil (U). Modified nucleobases include other synthetic and natural nucleobases.

Modified nucleobases include, but are not limited to, 5-substituted pyrimidines, 6-azapyrimidines, alkyl or alkynyl substituted pyrimidines, alkyl substituted purines, and N-2, N-6 and 0-6 substituted purines. In certain some embodiments, modified nucleobases are selected from: 5-methylcytosine, 2-aminopropyladenine, 5-hydroxymethyl cytosine, xanthine, hypoxanthine, deoxythimidine (dT), 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-Nbenzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808; The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; the entire contents of each of which is incorporated herein by reference for all purposes.

In some embodiments, the modified nucleobase comprises a pseudouridine, 2′thiouridine (s2U), N6′-methyladenosine, 5′methylcytidine (m⁵C), 5′fluoro-2′deoxyuridine, N-ethylpiperidine 7-EAA triazole modified adenine, N-ethylpiperidine 6′triazole modified adenine, 6-phenylpyrrolo-cytosine (PhpC), 2′,4′-difluorotoluyl ribonucleoside (rF), or 5′nitroindole. In some embodiments, the modified nucleobase comprises a 5-substituted pyrimidine; 6-azapyrimidine; or N-2, N-6 and 0-6 substituted purines (including 2-aminopropyladenine, 5-propynyluracil and 5-propynylcytosine). 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents an published applications that teach the preparation of certain of the above noted modified nucleobases as well as other modified nucleobases include, but are not limited to, U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; 7,495,088; 5,130,302; 5,134,066; 5,175,273; 5,367,066; 5,432,272; 5,434,257; 5,457,187; 5,459,255; 5,484,908; 5,502,177; 5,525,711; 5,552,540; U.S. Pat. Nos. 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,166,199; and 6,005,096, the entire contents of each of which is hereby incorporated herein by reference for all purposes.

### (c) Internucleoside Linkage Modifications

In some embodiments, the modified gRNA comprises one or more modified internucleoside linkage. Modified internucleoside linkages, compared to naturally occurring phosphate linkages, can be used to alter, typically increase, nuclease resistance of an agent (e.g., described herein).

The naturally occurring internucleoside linkage of RNA and DNA is a 3′ to 5′ phosphodiester linkage. In some embodiments, the modified internucleoside linkage contains a normal 3′-5′ linkage. In some embodiments, the modified internucleoside linkage contains a 2′-5′ linkage. In some embodiments, the modified internucleoside linkage has an inverted polarity wherein the adjacent pairs of nucleoside units are linked e.g., 3′-5′ to 5′-3′ or 2′-5′ to 5′-2′.

The two main classes of modified internucleoside linking can be defined by the presence or absence of a phosphorous atom.

### Modified Phosphorous Containing Internucleoside Linkages

In some embodiments, the modified internucleoside linkage comprises a phosphorous atom. Representative modified phosphorus-containing internucleoside linkages include but are not limited to phosphorothioates (PS (Rp isomer or Sp isomer)) (e.g., 5′phosphorothioate) (e.g., a chiral phosphorothioate), phosphotriesters, phosphoramidates (e.g., 3′-amino phosphoramidate and aminoalkylphosphoramidates), chiral phosphorothioates, phosphorodithioates (PS2), aminoalkylphosphotriesters, methyl and other alkyl phosphonates (e.g., methylphosphonate (MP), 3′-alkylene phosphonates), methpxypropyl-phosphonates (MOP), 5′-(E)-vinylphosphonates, 5′methyl phosphonates, (S)-5′C-methyl with phosphates, phosphinates, thionophosphoramidates, thionoalkylphosphonates, thionoalkylphosphotriesters, boranophosphates, phosphinates, and peptide nucleic acids (PNAs).

Methods of preparing polynucleotides containing one or more modified phosphorus-containing internucleoside linkage are known in the art. See, e.g., U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference for all purposes.

### Modified Non-Phosphorous Containing Internucleoside Linkages

In some embodiments, the modified internucleoside linkage does not contain a phosphorous atom. Modified internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative non-phosphorous containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—).

Methods of preparing polynucleotides comprising modified internucleoside linkages do not contain a phosphorous atom are known in the art. See, e.g., U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

### (d) Exemplary Combinations of Modifications

As described above, the recited exemplary modifications can be used in any (non-mutually exclusive combinations). For example, exemplary combinations of modifications include, 2′-O-Me 3′-phosphorothioate (MS) nucleotides; 2′-O-MOE 3′-phosphorothioate nucleotides; 2′-F 3′-phosphorothioate nucleotides; 2′-O-Me 3′-thioPACE (MSP) nucleotides; and 2′-deoxy 3′-phosphorothioate nucleotides.

### (ii) Location of Modifications

The modified nucleotides can be located at any suitable position throughout the gRNA (e.g., the terminal (e.g., 5′ terminal, 3′ terminal, or 5′ and 3′ terminal residues) of the full-length gRNA; any domain of the gRNA (e.g., the crRNA or tracrRNA of a sgRNA or a template RNA); internal residues of the full-length gRNA; etc).

In some embodiments, the terminal (e.g., 5′ terminal, 3′ terminal, or 5′ and 3′ terminal residues) of the gRNA are modified. In some embodiments, modification of the terminal residues reduces degradation of the gRNAs (e.g., in a cell) by exonucleases. In some embodiments, modification of the terminal residues increases stability of the gRNA (e.g., in a cell (e.g., in vitro, ex vivo, in vivo). In some embodiments, the 5′ terminus of the gRNA comprises one or more modified nucleotides. In some embodiments, the 5′ terminal 1, 2, 3, 4, or 5 nucleotides are modified. In some embodiments, the 3′ terminus of the gRNA comprises one or more modified nucleotides. In some embodiments, the 3′ terminal 1, 2, 3, 4, or 5 nucleotides are modified. In some embodiments, the 3′ terminus and the 5′ terminus of the gRNA comprises one or more modified nucleotides. In some embodiments, the 3′terminal 1, 2, 3, 4, or 5 nucleotides are modified and the 5′ terminal 1, 2, 3, 4, or 5 nucleotides are modified.

In some embodiments, one or more internal (i.e., non-terminal) nucleotides of the gRNA are modified. In some embodiments, modification of the internal residues reduces degradation of the gRNAs (e.g., in a cell) by endonucleases. In some embodiments, modification of the internal residues increases stability of the gRNA (e.g., in a cell (e.g., in vitro, ex vivo, in vivo). In some embodiments, at least 1, 2, 3, 4, 5, 6, 7, 8, 9, or 10, or more of the internal nucleotides of the gRNA are modified.

In some embodiments, one or more nucleotides of the crRNA (e.g., of a sgRNA of a template RNA) are modified. In some embodiments, one or more of the nucleotides of the seed region, the PAM-distal region, and/or the tracrRNA binding region of the crRNA (e.g., of a sgRNA of a template RNA) are modified. In some embodiments, the 3′ terminal and/or 5′ terminal nucleotides of the crRNA are modified. In some embodiments, at least 1, 2, 3, 4, 5, 6, 7, 8, 9, or 10, or more nucleotides of the crRNA (e.g., of a sgRNA of a template RNA) are modified. In some embodiments, one or more nucleotides of the tracrRNA (e.g., of a sgRNA of a template RNA) are modified. In some embodiments, one or more of the nucleotides of the tracrRNA (e.g., of a sgRNA of a template RNA) that do not interact with a Cas endonuclease (e.g., a Cas endonuclease described herein) are modified.

4.5.2.3 Methods of Making gRNAs

gRNAs can be generated according to standard nucleic acid synthesis methods known in the are described herein (see, e.g., § 4.6).

The generation of multi-domain gRNAs (e.g., sgRNAs, template gRNAs) may be assembled by the connection of two or more (e.g., two, three, four, five, six, seven, eight, nine, ten, or more) RNA segments with each other. For example, these gRNAs can be generated by contacting two or more linear RNA segments with each other under conditions that allow for the 5′ terminus of a first RNA segment to be covalently linked with the 3′ terminus of a second RNA segment. The joined molecule could be contacted with a third RNA segment under conditions that allow for the 5′ terminus of the joined molecule to be covalently linked with the 3′ terminus of the third RNA segment. The method could further comprise joining a fourth, fifth, or additional RNA segments to the elongated molecule. This form of assembly may, in some instances, allow for rapid and efficient assembly of gRNA molecules (e.g., multi region gRNAs (e.g., sgRNAs, template gRNAs)). See, e.g., US20160102322A1 (e.g., FIG. 10) and WO2021178720, the entire contents of each of which are incorporated herein by reference for all purposes.

In some embodiments, RNA segments may be produced by chemical synthesis. In some embodiments, RNA segments may be produced by in vitro transcription of a nucleic acid template, e.g., by providing an RNA polymerase to act on a cognate promoter of a DNA template to produce an RNA transcript. In some embodiments, in vitro transcription is performed using, e.g., a T7, T3, or SP6 RNA polymerase, or a derivative thereof, acting on a DNA, e.g., dsDNA, ssDNA, linear DNA, plasmid DNA, linear DNA amplicon, linearized plasmid DNA, e.g., encoding the RNA segment, e.g., under transcriptional control of a cognate promoter, e.g., a T7, T3, or SP6 promoter. In some embodiments, a combination of chemical synthesis and in vitro transcription is used to generate the RNA segments for assembly. In some embodiments, in vitro transcription may be better suited for the production of longer RNA molecules (as compared to chemical synthesis). In some embodiments, reaction temperature for in vitro transcription may be lowered, e.g., be less than 37° C. (e.g., between 0-10° C., 10-20° C., or 20-30° C.), to result in a higher proportion of full-length transcripts (Krieg Nucleic Acids Res 18:6463 (1990)). In some embodiments, a protocol for improved synthesis of long transcripts is employed to synthesize a long template RNA, e.g., a template RNA greater than 5 kb, such as the use of e.g., T7 RiboMAX Express, which can generate 27 kb transcripts in vitro (see, e.g., Thiel et al. J Gen Virol 82(6):1273-1281 (2001), the entire contents of which are incorporated herein by reference for all purposes). In some embodiments, modifications to RNA molecules as described herein may be incorporated during synthesis of RNA segments (e.g., through the inclusion of modified nucleotides or alternative binding chemistries), following synthesis of RNA segments through chemical or enzymatic processes, following assembly of one or more RNA segments, or a combination thereof.

Additional exemplary methods that may be used to connect RNA segments is by click chemistry (e.g., as described in U.S. Pat. Nos. 7,375,234; 7,070,941; US20130046084; and US20160102322A the entire contents of each of which are incorporated herein by reference for all purposes. Any click reaction may potentially be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). In some embodiments, ligation of RNA molecules using a click chemistry reaction is advantageous because click chemistry reactions are fast, modular, efficient, often do not produce toxic waste products, can be done with water as a solvent, and/or can be set up to be stereospecific.

### 4.5.3 Nucleic Acid Editing Activity of Systems

As described above, the systems described herein are useful in, inter alia, editing (e.g., the addition, deletion, or substitution of one or more nucleotide) a target nucleic acid molecule (e.g., DNA, genome, gene (e.g., within a cell, e.g., within a cell in a subject (e.g., a mammalian subject, e.g., a human subject))) (e.g., in vivo, ex vivo, or in vitro).

In some embodiments, the system (e.g., a system described herein comprising a Cas endonuclease described herein) exhibits increased editing efficiency relative to the editing efficiency of a reference system comprising reference Cas endonuclease. In some embodiments, the system (e.g., a system described herein comprising a Cas endonuclease described herein) exhibits at least about a 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more increase in editing efficiency relative to the editing efficiency of a reference system comprising reference Cas endonuclease. In some embodiments, the system (e.g., a system described herein comprising a Cas endonuclease described herein) exhibits at least about a 50%, 55%, 60%, 65%, 70%, 75%, 80%, 85%, 90%, 95%, 100%, or more increase in editing efficiency relative to the editing efficiency of a reference system comprising reference Cas endonuclease. In some embodiments, the system (e.g., a system described herein comprising a Cas endonuclease described herein) described herein exhibits an increase from about 30%-200%, 40%-200%, 50%-200%, 60%-200%, 70%-200%, 80%-200%, 90%-200%, 100%-200%, 150%-200%, 30%-150%, 40%-150%, 50%-150%, 60%-150%, 70%-150%, 80%-150%, 90%-150%, 100%-150%, 30%-100%, 40%-100%, 50%-100%, 60%-100%, 70%-100%, 80%-100%, or 90%-100%, or more increase in editing efficiency relative to the editing efficiency of a reference system comprising reference Cas endonuclease.

In some embodiments, the system (e.g., a system described herein comprising a Cas endonuclease described herein) exhibits increased editing efficiency relative to the editing efficiency of a system comprising the reference Cas endonuclease set forth in SEQ ID NO: 321. In some embodiments, the system (e.g., a system described herein comprising a Cas endonuclease described herein) exhibits at least about a 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, or more increase in editing efficiency relative to the editing efficiency of a system comprising the reference Cas endonuclease set forth in SEQ ID NO: 321. In some embodiments, the system (e.g., a system described herein comprising a Cas endonuclease described herein) exhibits at least about a 50%, 55%, 60%, 65%, 70%, 75%, 80%, 85%, 90%, 95%, 100%, or more increase in editing efficiency relative to the editing efficiency of a system comprising the reference Cas endonuclease set forth in SEQ ID NO: 321. In some embodiments, the system (e.g., a system described herein comprising a Cas endonuclease described herein) exhibits an increase from about 30%-200%, 40%-200%, 50%-200%, 60%-200%, 70%-200%, 80%-200%, 90%-200%, 100%-200%, 150%-200%, 30%-150%, 40%-150%, 50%-150%, 60%-150%, 70%-150%, 80%-150%, 90%-150%, 100%-150%, 30%-100%, 40%-100%, 50%-100%, 60%-100%, 70%-100%, 80%-100%, or 90%-100%, or more increase in editing efficiency relative to the editing efficiency of a system comprising the reference Cas endonuclease set forth in SEQ ID NO: 321.

### 4.5.4 Methods of Assessing Nucleic Acid Editing Activity of Systems

Standard methods of assessing the editing of a target nucleic acid molecule (e.g., in a cell) by a system described herein are known in the art and described herein. See, e.g., Maja Gehre et. al. Efficient strategies to detect genome editing and integrity in CRISPR-Cas9 engineered ESCs, bioRxiv 635151; doi: https://doi.org/10.1101/635151 Glaser A, McColl B, Vadolas J. GFP to BFP Conversion: A Versatile Assay for the Quantification of CRISPR/Cas9-mediated Genome Editing [published correction appears in Mol Ther Nucleic Acids. 2016 Sep. 13; 5(9):e360]. Mol Ther Nucleic Acids. 2016; 5(7):e334. Published 2016 Jul. 12. doi:10.1038/mtna.2016.48, the entire contents of each of which are incorporated by reference herein for all purposes. For example, standard nucleic acid sequencing methods (e.g., next generation sequencing, Sanger sequencing), assessment of a phenotype associated with a specific target edit, a mismatch detection assay, or a restriction fragment length polymorphism assay.

For example, for monitoring gene editing of a target DNA, mammalian cells, e.g., HEK293T or U2OS cells, carrying a target DNA may be utilized. In other embodiments for monitoring gene editing of a target DNA, mammalian cells, e.g., HEK293T or U2OS cells, carrying a target DNA genomic landing pad may be utilized. In particular embodiments, the target DNA genomic landing pad may comprise a gene to be edited for treatment of a disease or disorder of interest. In other particular embodiments, the target DNA is a gene sequence that expresses a protein that exhibits detectable characteristics that may be monitored to determine whether gene editing has occurred. For example, in certain embodiments, a blue fluorescence protein (BFP)—or green fluorescence protein (GFP)-expressing genomic landing pad is utilized. In certain embodiments, mammalian cells, e.g., HEK293T or U2OS cells, comprising a target DNA, e.g., a target DNA genomic landing pad, are seeded in culture plates at 500×-3000× cells per editing system and transduced at a 0.2-0.3 multiplicity of infection (MOI) to minimize multiple infections per cell. Puromycin (2.5 ug/mL) may be added 48 hours post infection to allow for selection of infected cells. In such an embodiment, cells may be kept under puromycin selection for at least 7 days and then scaled up for gRNA (e.g., template RNA) introduction (e.g., electroporation, e.g., template RNA electroporation).

To ascertain whether gene editing occurs, mammalian cells containing a target DNA to be edited may be infected with a candidate endonuclease (or a fusion protein thereof (e.g., a reverse-transcriptase based fusion protein)) then transfected with guide RNA (e.g., template RNA) designed for use in editing of the target DNA. Subsequently, the cells may be analyzed to determine whether editing of the target DNA has occurred according to the designed outcome, or whether no editing or imperfect editing has occurred, e.g., by using cell sorting and sequence analysis.

In a particular embodiment, to ascertain whether gene editing occurs, BFP—or GFP-expressing mammalian cells, e.g., HEK293T or U2OS cells, may be infected with a candidate endonuclease (or a fusion protein thereof (e.g., a reverse-transcriptase based fusion protein)) and then transfected or electroporated with guide RNA plasmid or RNA (e.g., template RNA plasmid or RNA), e.g., by electroporation of ˜250,000 cells/well with 200 ng of a guide RNA plasmid or RNA (e.g., template RNA plasmid or RNA) designed to convert BFP-to-GFP or GFP-to-BFP, at a cell count ensuring >250×-1000× coverage per candidate. In such an embodiment, the gene-editing capacity of the various constructs in this assay may be assessed by sorting the cells by Fluorescence-Activated Cell Sorting (FACS) for expression of the color-converted fluorescent protein (FP) at 4-10 days post-electroporation. Cells are sorted and harvested as distinct populations of unedited cells (exhibiting original florescence protein signal), edited cells (exhibiting converted fluorescence protein signal), and imperfect edit (exhibiting no florescence protein signal) cells. A sample of unsorted cells may also be harvested as the input population to determine candidate enrichment during analysis. The site of targeted editing may also be analyzed by standard sequencing (e.g., next-generation sequencing methods).

### 4.5.5 Exemplary Systems

Exemplary systems are provided below that incorporate components described above. The exemplary systems include exemplary homology directed repair (HDR) based editing systems; reverse transcriptase-based editing systems; and nucleobase editor-based editing systems. The systems are exemplary and not intended to be limiting.

### 4.5.5.1 HDR Based Editing Systems

Provided herein are, inter alia, HDR based systems (e.g., for use in editing target nucleic acid molecules, e.g., in cells, e.g., within a subject). In some embodiments, the system comprises (a) (i) a Cas endonuclease described herein (or a functional fragment, functional variant, or domain thereof); (ii) a fusion protein comprising a Cas endonuclease described herein (or a functional fragment or functional variant thereof) (e.g., described herein); (iii) a conjugate comprising a Cas endonuclease described herein (or a functional fragment or functional variant thereof) (e.g., described herein); (iv) a nucleic acid molecule encoding (a)(i), (a)(ii), or (a)(iii) (e.g., a nucleic acid molecule described herein); (v) a vector comprising (a)(iv) (e.g., a vector described herein); (vi) a carrier comprising any one of (a)(i)-(a)(v) (e.g., a carrier described herein); or (vii) a composition comprising any one of (a)(i)-(a)(vi) (e.g., a composition (e.g., a pharmaceutical composition) described herein); (b) (i) a gRNA comprising (i-a) a crRNA and a tracrRNA, wherein the crRNA and a tracrRNA are on separate nucleic acid molecules or (i-b) a sgRNA; (ii) one or more DNA molecule encoding (b) (i); (iii) a vector comprising (b)(i) or (b)(ii) (e.g., a vector described herein); (iv) a carrier comprising any one of (b)(i)-(b)(iii) (e.g., a carrier described herein); or (v) a composition (e.g., a pharmaceutical composition) comprising any one of (b)(i)-(b)(iv) (e.g., a composition (e.g., a pharmaceutical composition) described herein); and (c) (i) a donor template nucleic acid (e.g., DNA) molecule (e.g., as defined herein) (ii) a vector comprising (c)(i) (e.g., a vector described herein); (iii) a carrier comprising any one of (c)(i)-(c)(ii) (e.g., a carrier described herein); or (iv) a composition (e.g., a pharmaceutical composition) comprising any one of (c)(i)-(c)(iii) (e.g., a composition (e.g., a pharmaceutical composition) described herein).

Without wishing to be bound by theory, the HDR system can be utilized e.g., in methods of editing a target nucleic acid molecule (e.g., methods described herein), wherein the molecular machinery of the cell (e.g., in a subject, ex vivo, or in vitro) will utilize the donor template nucleic acid molecule in repairing and/or resolving a cleavage site in a target nucleic acid molecule mediated by a Cas endonuclease (or functional fragment, functional variant, or domain thereof) (e.g., of the system), wherein donor sequence will be incorporated into the target nucleic acid molecule through e.g., HDR. See, e.g., U.S. Pat. No. 8,697,359, the entire contents of which is incorporated herein by reference for all purposes.

In some embodiments, the endonuclease (or the functional fragment, functional variant, or domain thereof) has the ability to mediate double strand breaks in a target double stranded nucleic acid (e.g., DNA) molecule.

In some embodiments, the donor template nucleic acid molecule comprises at least about 10, 20, 30, 40, 50, 60, 70, 80, 90, 100, 150, 200, 300, 400, or 500 or more nucleotides. In some embodiments, the donor template nucleic acid molecule comprises from about 10-500, 10-400, 10-300, 10-200, 10-100, 10-90, 10-80, 10-70, 10-60, 10-50, 10-40, 10-30, or 10-20 nucleotides. In some embodiments, the donor template nucleic acid molecule comprises about 10, 20, 30, 40, 50, 60, 70, 80, 90, 100, 150, 200, 300, 400, or 500 or more nucleotides. In some embodiments, the donor sequence of the donor template nucleic acid molecule comprises a substitution, addition, deletion, inversion, or another modification (e.g., relative to the nucleotide sequence of the target nucleic acid molecule).

In some embodiments, each homology arm of the donor template nucleic acid molecule comprises at least about 10, 15, 20, 30, 40, 50, 60, 70, 80, 90, 100, 200, or 300 nucleotides. In some embodiments, each homology arm of the donor template nucleic acid molecule comprises from about 10-300, 10-200, 10-100, 10-90, 10-80, 10-70, 10-60, 10-50, 10-40, 10-30, 10-20, or 10-15 nucleotides. In some embodiments, each homology arm of the donor template nucleic acid molecule comprises about 10, 15, 20, 30, 40, 50, 60, 70, 80, 90, 100, 200, or 300 nucleotides. In some embodiments, each homology arm shares at least about 70%, 80%, 85%, 90%, 91%, 92%, 93%, 94%, 95%, 96%, 97%, 98%, 99%, or 100% sequence homology to its target sequence. In some embodiments, the target sequence of the homology arms is immediately flanking the endonuclease cleavage site. In some embodiments, the target sequence of the homology arms is within about 1, 2, 3, 4, 5, 6, 7, 8, 9, 10, 15, 20, or 30 nucleotides of the endonuclease cleavage site.

In some embodiments, the donor template nucleic acid molecule is a ssDNA molecule, ssRNA molecule, dsDNA molecule, or dsRNA molecule. In some embodiments, the donor template nucleic acid molecule of the system is a linear nucleic acid molecule. In some embodiments, the donor template nucleic acid molecule of is a circular nucleic acid molecule. In some embodiments, the donor template nucleic acid molecule of comprised in a vector and/or carrier. In some embodiments, the donor template nucleic acid molecule of comprises one or more modified nucleotides. Nucleotide modifications are known in the art and described herein. For example, one or more nucleotides may be modified to increase stability, decrease degradation (e.g., by endonucleases and/or exonucleases). Exemplary modifications include, but are not limited to, 2′-O-methyl (2′-OMe); 2′O-methoxyethyl (2′-O-MOE); 2′deoxy-2′-fluoro (2′-F); 2′-arabino-fluoro (2′-Ara-F); 2′-O-benzyl; 2′-O-methyl-4-pyridine (2-O-methyl-4-pyridine (2′-O—CH₂Py(4)); 2′F-4′-Cα-OMe; or 2′,4′-di-Cα-OMe, deoxyribose, phosphorothioates (PS (Rp isomer or Sp isomer)) (e.g., 5′phosphorothioate) (e.g., a chiral phosphorothioate), phosphotriesters, phosphoramidates (e.g., 3′-amino phosphoramidate and aminoalkylphosphoramidates), chiral phosphorothioates, phosphorodithioates (PS2), aminoalkylphosphotriesters, methyl and other alkyl phosphonates (e.g., methylphosphonate (MP), 3′-alkylene phosphonates), methpxypropyl-phosphonates (MOP), 5′-(E)-vinylphosphonates, 5′methyl phosphonates, (S)-5′C-methyl with phosphates, phosphinates, thionophosphoramidates, thionoalkylphosphonates, thionoalkylphosphotriesters, boranophosphates, phosphinates, and peptide nucleic acids (PNAs), and any combination thereof. See, also, § 4.5.2.2 herein, which describes modified gRNAs. Any of the modifications described in § 4.5.2.2 may also be utilized in the context of a donor template nucleic acid molecule.

In some embodiments, the donor sequence of the donor template nucleic acid molecule comprises e.g., restriction sites, nucleotide polymorphisms, selectable markers (e.g., drug resistance genes, fluorescent proteins, enzymes etc.), etc., which may be used to assess for successful addition of the donor sequence of the donor template nucleic acid molecule at the cleavage site or in some cases may be used for other purposes (e.g., to signify expression at the target nucleic acid sequence (e.g., gene)). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

### 4.5.5.2 RT Based Editing Systems

Provided herein are, inter alia, RT based systems (e.g., for use in editing target nucleic acid molecules, e.g., in cells, e.g., within a subject). In some embodiments, the system comprises (a) (i) a fusion protein comprising a Cas endonuclease described herein (or a functional fragment, functional variant, or domain thereof) (e.g., described herein) and a reverse transcriptase (or a functional fragment, functional variant, or domain thereof) (e.g., described herein) (see, e.g., § 4.3.1.1); (ii) a nucleic acid molecule encoding (a)(i) (e.g., a nucleic acid molecule described herein); (iii) a vector comprising (a)(ii) (e.g., a vector described herein); (iv) a carrier comprising any one of (a)(i)-(a)(iii) (e.g., a carrier described herein); or (v) a composition comprising any one of (a)(i)-(a)(iv) (e.g., a composition (e.g., a pharmaceutical composition) described herein); and (b) (i) a template RNA (e.g., described herein) (see, e.g., § 4.5.2); (ii) a DNA molecule encoding (b)(i); (iii) a vector comprising (b)(i) or (b)(ii) (e.g., a vector described herein); (iv) a carrier comprising any one of (b)(i)-(b)(iii) (e.g., a carrier described herein); or (v) a composition comprising any one of (b)(i)-(b)(iv) (e.g., a composition (e.g., a pharmaceutical composition) described herein).

Without wishing to be bound by theory, the RT based editing system can be utilized e.g., in methods of editing a target nucleic acid molecule (e.g., methods described herein), wherein the template nucleic acid binds to a target nucleic acid molecule (e.g., a double stranded nucleic acid molecule (e.g., a dsDNA molecule)) and binds to the fusion protein to thereby localize the fusion protein to the target nucleic acid molecule. Subsequently the Cas endonuclease of the fusion protein cleaves the target nucleic acid molecule (e.g., a single strand of a target double stranded nucleic acid molecule (e.g., a dsDNA molecule)) allowing the 3′ homology domain to bind a sequence adjacent to the site to be edited on the target nucleic acid molecule (e.g., on the edited strand of a double stranded nucleic acid molecule (e.g., a dsDNA molecule)). It is thought that the reverse transcriptase domain of the fusion protein utilizes the 3′ target homology domain as a primer and the edit template as a template to, e.g., polymerize a sequence complementary to the edit template. Without wishing to be bound by theory, it is thought that selection of an appropriate edit template can result in editing of the nucleotide sequence of the target site (e.g., the substitution, deletion, or addition of one or more nucleotides at the target site), wherein a cell's endogenous DNA repair machinery resolves the mismatched double stranded nucleic acid molecule (e.g., dsDNA) to incorporate the desired edit. See, e.g., WO2021178720 and WO2023039424, the entire contents of each of which are incorporated herein by reference for all purposes.

In some embodiments, the Cas endonuclease (a) has the ability to mediate single strand breaks in a target double stranded nucleic acid (e.g., DNA) molecule; (b) is not able to mediate double strand breaks in a target double stranded nucleic acid (e.g., DNA) molecule; (c) has the ability to mediate single strand breaks in a target double stranded nucleic acid (e.g., DNA) molecule and is not able to mediate double strand breaks in a target double stranded nucleic acid (e.g., DNA) molecule (i.e., nickase activity); and/or (d) has RNA guided DNA endonuclease activity; or any combination of the foregoing.

In some embodiments, the target nucleic acid molecule of the system is a double stranded nucleic acid (e.g., dsDNA) molecule, wherein one strand of the double stranded nucleic acid (e.g., dsDNA) molecule is targeted for editing. In some embodiments, the system further comprises a gRNA (e.g., sgRNA) that is capable of directing the Cas endonuclease (e.g., described herein) of the system to form a single strand break (i.e., a nick) in the non-edited strand of a target double stranded nucleic acid (e.g., dsDNA) molecule. Without wishing to be bound by theory it is thought that the nicking of the non-edited strand of a target double stranded nucleic acid molecule (e.g., a target dsDNA molecule) induces preferential replacement of the edited strand. In some embodiments, at least a portion of the nucleotide sequence of the gRNA (e.g., sgRNA) is complementary to a portion of the nucleotide sequence of the edited strand (as defined herein) of the target double stranded nucleic acid (e.g., dsDNA) molecule. In some embodiments, at least a portion of the nucleotide sequence of the second gRNA (e.g., sgRNA) binds to a portion of the nucleotide sequence of the edited strand (as defined herein) of a double stranded nucleic acid (e.g., dsDNA) molecule. In some embodiments, the gRNA is a sgRNA. In some embodiments, the gRNA (e.g., sgRNA) is present on the same nucleic acid molecule as the template gRNA (or the nucleic acid (e.g., DNA) molecule encoding the gRNA is present on the same nucleic acid (e.g., DNA) molecule encoding the template gRNA). In some embodiments, the gRNA (e.g., sgRNA) is present on a different nucleic acid molecule as the template gRNA (or the nucleic acid (e.g., DNA) molecule encoding the gRNA is present on a different nucleic acid (e.g., DNA) molecule encoding the template gRNA).

In some embodiments, a Cas endonuclease described herein (or a functional fragment, functional variant, or domain thereof) is utilized in a system (e.g., a Gene Writer™ system) described in WO2021178720 or WO2023039424, the entire contents of each of which are incorporated herein by reference for all purposes.

### 4.5.5.3 Nucleobase Editor Editing Systems

Provided herein are, inter alia, nucleobase editor-based systems (e.g., for use in editing target nucleic acid molecules, e.g., in cells, e.g., within a subject). In some embodiments, the system comprises (a) (i) a fusion protein comprising a Cas endonuclease described herein (or a functional fragment or functional variant thereof) (e.g., described herein) and a nucleobase editor (or a functional fragment or functional variant thereof) (e.g., described herein) (see, e.g., § 4.3.1.2); (ii) a nucleic acid molecule encoding (a)(i) (e.g., a nucleic acid molecule described herein); (iii) a vector comprising (a)(ii) (e.g., a vector described herein); (iv) a carrier comprising any one of (a)(i)-(a)(iii) (e.g., a carrier described herein); or (v) a composition comprising any one of (a)(i)-(a)(iv) (e.g., a composition (e.g., a pharmaceutical composition) described herein); and (b) (i) a first gRNA comprising (i-a) a crRNA and a tracrRNA, wherein the crRNA and a tracrRNA are one separate nucleic acid molecules or (i-b) a sgRNA; (ii) one or more DNA molecule encoding (b) (i); (iii) a vector comprising (b)(i) or (b)(ii) (e.g., a vector described herein); (iv) a carrier comprising any one of (b)(i)-(b)(iii) (e.g., a carrier described herein); or (v) a composition comprising any one of (b)(i)-(b)(iv) (e.g., a composition (e.g., a pharmaceutical composition) described herein).

Without wishing to be bound by theory, the nucleobase editor based editing system can be utilized e.g., in methods of editing a target nucleic acid molecule (e.g., methods described herein), wherein the gRNA (e.g., sgRNA) nucleic acid binds to a target nucleic acid molecule (e.g., a double stranded nucleic acid molecule (e.g., a dsDNA molecule) and binds to the fusion protein to thereby localize the fusion protein to the target nucleic acid molecule. Subsequently the endonuclease (e.g., nickase) of the fusion protein cleaves the target nucleic acid molecule (e.g., a single strand of a target double stranded nucleic acid molecule (e.g., a dsDNA molecule)) allowing the nucleobase editor (e.g., deaminase) to edit one more nucleobase in the nucleotide sequence of the target nucleic acid molecule (e.g., in a single strand of a target double stranded nucleic acid molecule (e.g., a dsDNA molecule) (i.e., the edited strand)). See, e.g., WO2021050571A1; WO2022/204268; WO2019079347A1, the entire contents of each of which is incorporated herein by reference for all purposes.

In some embodiments, the Cas endonuclease (a) has the ability to mediate single strand breaks in a target double stranded nucleic acid (e.g., DNA) molecule; (b) is not able to mediate double strand breaks in a target double stranded nucleic acid (e.g., DNA) molecule; (c) has the ability to mediate single strand breaks in a target double stranded nucleic acid (e.g., DNA) molecule and is not able to mediate double strand breaks in a target double stranded nucleic acid (e.g., DNA) molecule (i.e., nickase activity); and/or (d) has RNA guided DNA endonuclease activity; or any combination of the foregoing.

In some embodiments, the target nucleic acid molecule of the system is a double stranded nucleic acid (e.g., dsDNA) molecule, wherein one strand of the double stranded nucleic acid (e.g., dsDNA) molecule is targeted for editing. In some embodiments, the system further comprises a gRNA (e.g., sgRNA) that is capable of directing the endonuclease (e.g., described herein) of the system to form a single strand break (i.e., a nick) in the non-edited strand of a target double stranded nucleic acid (e.g., dsDNA) molecule. Without wishing to be bound by theory it is thought that the nicking of the non-edited strand of a target double stranded nucleic acid molecule (e.g., a target dsDNA molecule) induces preferential replacement of the edited strand. In some embodiments, at least a portion of the nucleotide sequence of the gRNA (e.g., sgRNA) is complementary to a portion of the nucleotide sequence of the edited strand (as defined herein) of the target double stranded nucleic acid (e.g., dsDNA) molecule. In some embodiments, at least a portion of the nucleotide sequence of the second gRNA (e.g., sgRNA) binds to a portion of the nucleotide sequence of the edited strand (as defined herein) of a double stranded nucleic acid (e.g., dsDNA) molecule. In some embodiments, the gRNA is a sgRNA. In some embodiments, the gRNA (e.g., sgRNA) is present on the same nucleic acid molecule as the template gRNA (or the nucleic acid (e.g., DNA) molecule encoding the gRNA is present on the same nucleic acid (e.g., DNA) molecule encoding the template gRNA). In some embodiments, the gRNA (e.g., sgRNA) is present on a different nucleic acid molecule as the template gRNA (or the nucleic acid (e.g., DNA) molecule encoding the gRNA is present on a different nucleic acid (e.g., DNA) molecule encoding the template gRNA).

### 4.6 Nucleic Acid Molecules

Further provided herein are nucleic acid (e.g., DNA, RNA) molecules encoding any protein described herein (e.g., a Cas endonuclease (or a functional fragment, functional variant, or domain thereof), a heterologous protein (e.g., a reverse transcriptase, a nucleobase editor), a fusion protein, a conjugate, or any RNA molecule described herein (e.g., a gRNA (e.g., a sgRNA, a template RNA)). Nucleic acid molecules described herein can be generated using common methods known in the art (e.g., chemical synthesis).

In some embodiments, the nucleic acid molecule is DNA. In some embodiments, the nucleic acid molecule is RNA (e.g., mRNA or circular RNA). In some embodiments, the nucleic acid (e.g., RNA) molecule is a translatable RNA. In some embodiments, the nucleic acid molecule is single stranded. In some embodiments the nucleic acid molecule is double stranded. In some embodiments, the nucleic acid molecule is a single stranded RNA molecule. In some embodiments, the nucleic acid molecule is a single stranded DNA molecule. In some embodiments, the nucleic acid molecule is a double stranded RNA molecule. In some embodiments, the nucleic acid molecule is a double stranded DNA molecule.

In some embodiments, the nucleic acid molecule is a linear coding nucleic acid construct. In some embodiments, the nucleic acid molecule is contained within a vector (e.g., a plasmid, a viral vector). In some embodiments, the nucleic acid molecule is contained within a non-viral vector. In some embodiments, the nucleic acid molecule is contained within a plasmid. In some embodiments, the nucleic acid molecule is contained within a viral vector. A more detailed description of vectors (e.g., non-viral (e.g., plasmids) and viral) for both RNA and DNA nucleic acids is provided in § 4.7.

In some embodiments, the nucleic acid molecule may be modified (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc. Modifications are known in the art and described herein (see, e.g., § 4.5.2.2).

In some embodiments, the nucleotide sequence of the nucleic acid molecule is codon optimized, e.g., for expression. In some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g. glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 4.7 Vectors

In some embodiments, a nucleic acid (DNA, RNA) molecule described herein is contained in a vector (e.g., a non-viral vector (e.g., a plasmid), a viral vector). As such, provided herein are vectors (e.g., non-viral vectors (e.g., plasmids) viral vectors) comprising one or more nucleic acid molecule described herein (e.g., nucleic acid molecules encoding any protein described herein (e.g., a Cas endonuclease (or a functional fragment, functional variant, or domain thereof), a heterologous protein (e.g., a reverse transcriptase, a nucleobase editor), a fusion protein, a conjugate, etc.) or any RNA molecule described herein (e.g., a gRNA (e.g., a sgRNA, a template RNA)) (e.g., see, e.g., § 4.6) are provided. Such vectors can be easily manipulated by methods well known to the ordinary person of skill in the art. The vector used can be any vector that is suitable for cloning nucleic acid molecules that can be used for transcription of the nucleic acid molecule of interest.

In some embodiments, the vector is a plasmid. A person of ordinary skill in the art is aware of suitable plasmids for expression of the DNA of interest. For example, plasmid DNA may be generated to allow efficient production of the encoded endonucleases in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

In some embodiments, the vector is a viral vector. Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. Exemplary, simian adenovirus vectors include AdCh63 (see, e.g., WO2005071093, the entire contents of which is incorporated by reference herein for all purposes) or AdCh68.

Viral vectors can be generated with a packaging/producer cell line (e.g., a mammalian cell line) using standard methods known to the person of ordinary skill in the art. Generally, a nucleic acid construct (e.g., a plasmid) encoding the transgene (e.g., a Cas endonuclease described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™, pCR3.1™, pCMV™, pFRT™ pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation,). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

### 4.8 Carriers

In some embodiments, a Cas endonuclease (or a functional fragment, functional variant, or domain thereof) described herein (see, e.g., § 4.2), a fusion protein described herein (see, e.g., § 4.3); a conjugate described herein (see, e.g., § 4.3; a system described herein (see, e.g., § 4.5) (or any one or more component thereof); a nucleic acid molecule described herein (see, e.g., § 4.6); a vector described herein (see, e.g., § 4.7); a cell described herein (see, e.g., § 4.9); a reaction mixture described herein (see, e.g., § 4.10), or a pharmaceutical composition described herein (see, e.g., § 4.11) is formulated within one or more carrier.

As such, the disclosure provides, inter alia, carriers comprising any one or more of the following: a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a cell described herein (see, e.g., § 4.9); areaction mixture described herein (see, e.g., § 4.10), or a pharmaceutical composition described herein (see, e.g., § 4.11).

Any of the foregoing (e.g., proteins, nucleic acid molecules, vectors, etc.) can be encapsulated within a carrier, chemically conjugated to a carrier, associated with the carrier. In this context, the term “associated” refers to the essentially stable combination of any one of the foregoing, e.g., a protein, nucleic acid molecule, etc., with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of any one of the foregoing, e.g., a protein, a nucleic acid molecule, etc.) into a carrier (e.g., a lipid-based carrier, e.g., an LNP, liposome, lipoplex, and/or nanoliposome) wherein the molecule (e.g., the protein, nucleic acid molecule, etc.) is entirely contained within the interior space of the carrier (e.g., the lipid-based carrier, e.g., the LNP, liposome, lipoplex, and/or nanoliposome).

Exemplary carriers include, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 4.8.1.

### 4.8.1 Lipid Based Carriers

In some embodiments, a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a cell described herein (see, e.g., § 4.9); a reaction mixture described herein (see, e.g., § 4.10), or a pharmaceutical composition described herein (see, e.g., § 4.11) is encapsulated or associated with one or more lipids (e.g., cationic lipids and/or neutral lipids), thereby forming lipid-based carriers such as lipid nanoparticles (LNPs), liposomes, lipoplexes, or nanoliposomes.

In some embodiments, any of the foregoing molecules (e.g., proteins, nucleic acid molecules, vectors, systems, etc.) is encapsulated in one or more lipids (e.g., cationic lipids and/or neutral lipids), thereby forming lipid-based carriers such as lipid nanoparticles (LNPs), liposomes, lipoplexes, or nanoliposomes. In some embodiments, the molecule (e.g., the protein, nucleic acid molecule, vector, system, etc.) is associated with one or more lipids (e.g., cationic lipids and/or neutral lipids), thereby forming lipid-based carriers such as lipid nanoparticles (LNPs), liposomes, lipoplexes, or nanoliposomes. In some embodiments, the molecule (e.g., the protein, nucleic acid molecule, vector, system, etc.) is encapsulated in LNPs (e.g., as described herein). The use of LNPs for mRNA delivery is further detailed in e.g., Hou X et al. Lipid nanoparticles for mRNA delivery. Nat Rev Mater. 2021; 6(12):1078-1094. doi: 10.1038/s41578-021-00358-0. Epub 2021 Aug. 10. PMID: 34394960; PMCID: PMC8353930, the entire contents of each of which are incorporated by reference herein for all purposes.

The molecules (e.g., the proteins, nucleic acid molecules, vectors, systems, etc.) described herein may be completely or partially located in the interior space of the LNPs, liposomes, lipoplexes, and/or nanoliposomes, within the lipid layer/membrane, or associated with the exterior surface of the lipid layer/membrane. One purpose of incorporating the molecule (e.g., the protein, nucleic acid molecule, vector, system, etc.) into LNPs, liposomes, lipoplexes, and/or nanoliposomes is to protect the molecule (e.g., the protein, nucleic acid molecule, vector, system, etc.) from an environment which may contain enzymes or chemicals or conditions that degrade the molecule (e.g., the protein, nucleic acid molecule, vector, system, etc.) from molecules or conditions that cause the rapid excretion of the molecule (e.g., the protein, nucleic acid molecule, vector, system, etc.). Moreover, incorporating the molecules (e.g., the proteins, nucleic acid molecules, vectors, systems, etc.) into LNPs, liposomes, lipoplexes, and/or nanoliposomes may promote the uptake of the molecules (e.g., the proteins, nucleic acid molecules, vectors, systems, etc.), and hence, may enhance the therapeutic effect of the proteins or nucleic acid molecules (e.g., RNA, e.g., mRNA). Accordingly, incorporating a molecule (e.g., protein, nucleic acid molecule, vector, system, etc.), into LNPs, liposomes, lipoplexes, and/or nanoliposomes may be particularly suitable for a pharmaceutical composition described herein, e.g., for intramuscular and/or intradermal administration.

In some embodiments, molecules (e.g., the proteins, nucleic acid molecules, vectors, systems, etc.) described herein are formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more molecules described herein (e.g., a protein, a nucleic acid molecule, a vector, a system, etc. described herein), or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more molecules (e.g., the proteins, nucleic acid molecules, vectors, systems, etc.) described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### 4.8.1.1 Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, the entire contents of which are incorporated by reference herein for all purposes. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, the entire contents of which are incorporated by reference herein for all purposes.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

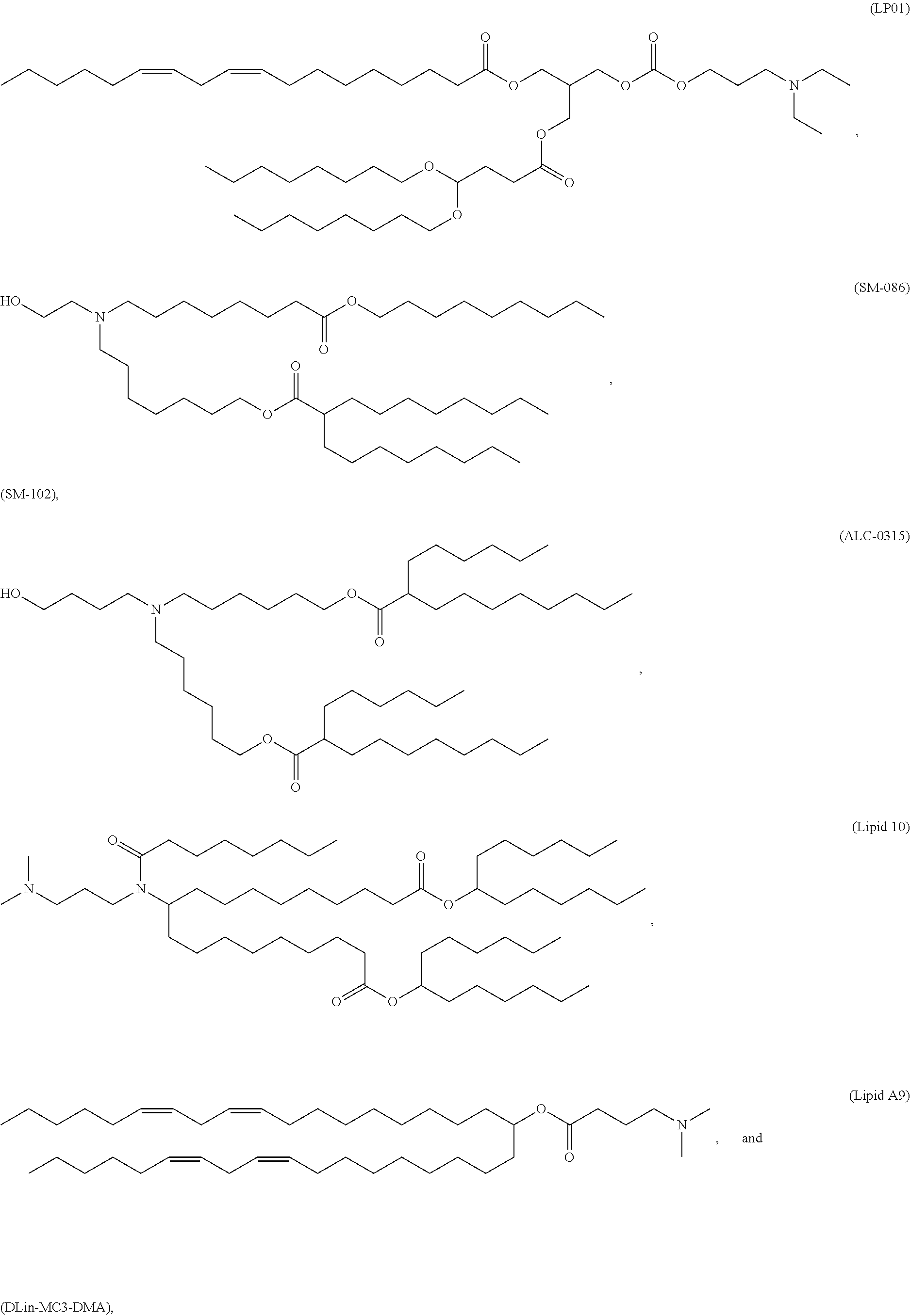

(see WO2017004143A1, the entire contents of which is incorporated herein by reference for all purposes).

In some embodiments, the lipid-based carrier (or lipid nanoformulation) further comprises one or more compounds described by WO 2021/113777 (e.g., a lipid of Formula (3) such as a lipid of Table 3 of WO 2021/113777), the entire contents of which are incorporated by reference herein for all purposes.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (the entire contents of which are incorporated by reference herein for all purposes).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y—Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, the entire contents of each of which are incorporated by reference herein for all purposes.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, the entire contents of each of which are incorporated by reference herein for all purposes.

### 4.8.1.2 Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, the entire contents of which are incorporated by reference herein for all purposes. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, the entire contents of each of which are incorporated by reference herein for all purposes.

In one embodiment, the lipid-based carrier (or lipid nanoformulation) further comprises one or more non-cationic lipid that is oleic acid or a compound of Formula I, II, or IV of US 2018/0028664, the entire contents of which are incorporated by reference herein for all purposes.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### 4.8.1.3 Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, the entire contents of each of which are incorporated by reference herein for all purposes. Additional exemplary sterols include phytosterols, including those described in Eygeris et al. (2020), Nano Lett. 2020; 20(6):4543-4549, the entire contents of which are incorporated by reference herein for all purposes.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, the entire contents of each of which are incorporated by reference herein for all purposes.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### 4.8.1.4 Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, the entire contents of which are incorporated by reference herein for all purposes). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (the entire contents of which are incorporated by reference herein for all purposes), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, the entire contents of each of which are incorporated by reference herein for all purposes.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, the entire contents of each of which are incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

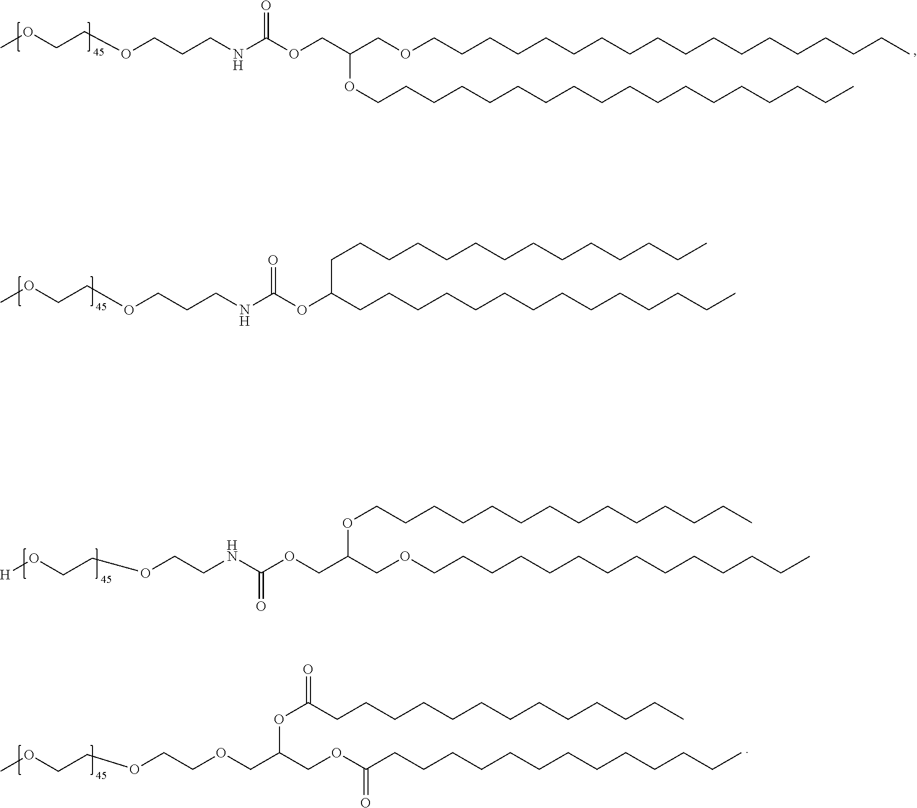

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, the entire contents of which are incorporated by reference herein for all purposes.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), the entire contents of each of which are incorporated by reference herein for all purposes.

### 4.8.1.5 Percentages of Lipid Nanoformulation Components

In some embodiments, the lipid-based carrier (or lipid nanoformulation) comprises one of more of the molecules described herein (e.g., a protein, a nucleic acid molecule, a vector, a system, etc. described herein), optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a payload (e.g., a molecule described herein (e.g., a protein, a nucleic acid molecule, a vector, a system, etc. described herein)). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a payload (e.g., a molecule described herein (e.g., a protein, a nucleic acid molecule, a vector, a system, etc. described herein)) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the payload may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a payload (e.g., a molecule described herein (e.g., a protein, a nucleic acid molecule, a vector, a system, etc. described herein)) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the payload may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a payload (e.g., a molecule described herein (e.g., a protein, a nucleic acid molecule, a vector, a system, etc. described herein)) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the payload may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a payload (e.g., a molecule described herein (e.g., a protein, a nucleic acid molecule, a vector, a system, etc. described herein)) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the payload may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a payload (e.g., a molecule described herein (e.g., a protein, a nucleic acid molecule, a vector, a system, etc. described herein)) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the payload may be at least 70%.

In some embodiments, the lipid-based carrier (or lipid nanoformulation) comprises (i) a molecule described herein (e.g., a protein, a nucleic acid molecule, a vector, a system, etc. described herein); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a molecule described herein (e.g., a protein, a nucleic acid molecule, a vector, a system, etc. described herein); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the payload (e.g., an encapsulated therapeutic agent such as a molecule described herein (e.g., a protein, a nucleic acid molecule, a vector, a system, etc. described herein) can be varied as desired. For example, the total lipid components to the payload (mass or weight) ratio can be from about 10:1 to about 30:1. In some embodiments, the total lipid components to the payload ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the payload can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of a payload such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%. 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### 4.9 Cells

The disclosure provides, inter alia, cells (e.g., host cells) comprising any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10), a carrier described herein (see, e.g., § 4.8); or a pharmaceutical composition described herein (see, e.g., § 4.11).

In some embodiments, the cell is a eukaryotic cell. In some embodiments, the cell is mammalian cell. In some embodiments, the cell is an animal cell. In some embodiments, the cell is a human cell. In some embodiments, the cell is in vitro. In some embodiments, the cell is in vivo. In some embodiments, the cell is ex vivo.

Standard methods known in the art can be utilized to deliver any one of the foregoing (e.g., endonuclease, fusion protein, system, vector, carrier, etc.) in a cell (e.g., a host cell). Standard methods known in the art can be utilized to culture cells (e.g., host cells) in vitro or ex vivo.

### 4.10 Reaction Mixtures

The disclosure provides, inter alia, reaction mixtures comprising any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a carrier described herein (see, e.g., § 4.8); or a pharmaceutical composition described herein (see, e.g., § 4.11).

In some embodiments, the reaction mixture comprises a target nucleic acid molecule (e.g., described herein). In some embodiments, the target nucleic acid molecule comprises a DNA molecule. In some embodiments, the target nucleic acid molecule comprises a dsDNA molecule. In some embodiments, the target nucleic acid molecule is a gene or genome. In some embodiments, the target nucleic acid molecule (e.g., a target DNA molecule (e.g., a target gene or genome)) is within a cell. In some embodiments, the cell is in vitro, ex vivo, or in vivo. In some embodiments the cells is a eukaryotic cell (e.g., a mammalian cell, an animal cell, a primate cell, a non-human primate cell, a human cell). In some embodiments, the cell is a human cell.

### 4.11 Pharmaceutical Compositions

The disclosure provides, inter alia, pharmaceutical compositions comprising any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and a pharmaceutically acceptable excipient (see, e.g., Remington's Pharmaceutical Sciences (1990) Mack Publishing Co., Easton, PA, the entire contents of which is incorporated by reference herein for all purposes).

The disclosure provides, inter alia, methods of making pharmaceutical compositions described herein comprising any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and formulating it into a pharmaceutically acceptable composition by the addition of one or more pharmaceutically acceptable excipient.

Also provided herein are pharmaceutical compositions comprising any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8), wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

A pharmaceutical composition described herein may be formulated for any route of administration to a subject. Non-limiting embodiments include parenteral administration, such as intramuscular, intradermal, subcutaneous, transcutaneous, or mucosal administration. In some embodiments, the pharmaceutical composition is formulated for administration by intramuscular, intradermal, or subcutaneous injection. In some embodiments, the pharmaceutical composition is formulated for administration by intramuscular injection. In some embodiments, the pharmaceutical composition is formulated for administration by intradermal injection. In some embodiments, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions is formulated as a multi-dose.

Acceptable excipients (e.g., carriers and stabilizers) compatible for inclusion in pharmaceutical compositions described herein are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG). Pharmaceutically acceptable excipients further include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

In some embodiments, a precise dose to be employed in a pharmaceutical composition (e.g., described herein) will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 4.12 Kits

The disclosure provides, inter alia, kits comprising any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or a pharmaceutical composition described herein (see, e.g., § 4.11).

In addition, a kit may comprise a liquid vehicle for solubilizing or diluting, and/or technical instructions. The technical instructions of the kit may contain information about administration and dosage and subject groups.

In some embodiments, the endonuclease (or a functional fragment, functional variant, or domain thereof) described herein, the fusion protein described herein; the conjugate described herein; the system described herein (or any one or more component thereof); the nucleic acid molecule described herein; the vector described herein; the reaction mixture described herein; the carrier described herein; and/or the pharmaceutical composition described herein is provided in a separate part of the kit. In some embodiments, the endonuclease (or a functional fragment, functional variant, or domain thereof) described herein, the fusion protein described herein; the conjugate described herein; the system described herein (or any one or more component thereof); the nucleic acid molecule described herein; the vector described herein; the reaction mixture described herein; the carrier described herein; and/or the pharmaceutical composition described herein is optionally lyophilized, spray-dried, or spray-freeze dried. The kit may further contain as a part a vehicle (e.g., buffer solution) for solubilizing the dried or lyophilized endonuclease (or a functional fragment, functional variant, or domain thereof) described herein, fusion protein described herein; conjugate described herein; system described herein (or any one or more component thereof); nucleic acid molecule described herein; vector described herein; reaction mixture described herein; carrier described herein; and/or pharmaceutical composition described herein.

In some embodiments, a kit comprises a single dose container. In some embodiments, the kit comprises a multi-dose container. In some embodiments, the kit comprises an administration device (e.g., an injector for intradermal injection or a syringe for intramuscular injection).

Any of the kits described herein may be used in any of the methods described herein (see, e.g., § 4.13).

### 4.13 Methods of Use

The disclosure provides, inter alia, various methods of utilizing any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); a pharmaceutical composition described herein (see, e.g., § 4.11); and/or a kit described herein (see, e.g., § 4.12).

In some embodiments, methods described herein comprise delivering, contacting, or introducing any one or more of the foregoing into a cell. Exemplary cells include, but are not limited to, e.g., eukaryotic cells, prokaryotic cells, animal cells, mammalian cells, primate cells, non-human primate cells, and human cells. In some embodiments, the cell is a eukaryotic cell, e.g., a cell of a multicellular organism, e.g., an animal, e.g., a mammal (e.g., human, swine, bovine) a bird (e.g., poultry, such as chicken, turkey, or duck), or a fish. In some embodiments, the cell is a non-human animal cell (e.g., a laboratory animal, a livestock animal, or a companion animal). In some embodiments, the cell is a stem cell (e.g., a hematopoietic stem cell), a fibroblast, or a T cell. In some embodiments, the cell is a non-dividing cell, e.g., a nondividing fibroblast or non-dividing T cell. In some embodiments, the cell is a eukaryotic cell (e.g., a mammalian cell, an animal cell, a primate cell, a non-human primate cell, a human cell). In some embodiments, the cell is a human cell. In some embodiments, the cell is a plant cell. In some embodiments, the cell is euploid, is not immortalized, is part of a tissue, is part of an organism, is a primary cell, is non-dividing, is haploid (e.g., a germline cell), is a non-cancerous polyploid cell, or is from a subject having a genetic disease. In some embodiments, the cell is in vitro, ex vivo, in vivo. In some embodiments, the cell is within a subject. In some embodiments, the subject described herein. In some embodiments, the subject is a mammal, animal, non-human primate, primate, human, or plant. In some embodiments, the subject is a human. In some embodiments, the cell is subsequently administered to a subject (e.g., for a therapeutic application (e.g., described herein (e.g., gene therapy))).

In some embodiments, methods described herein comprise administering any one or more of the foregoing to a subject. Exemplary subjects include, but are not limited to, e.g., mammals, e.g., humans, non-human mammals, e.g., non-human primates. In some embodiments, the subject is a human. In some embodiments, the subject is a vertebrate animal (e.g., mammal, bird, fish, reptile, or amphibian). In some embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

The dosage of any of the foregoing, to be administered to a subject in accordance with any of the methods described herein can be determined in accordance with standard techniques known to those of ordinary skill in the art, including the route of administration, the age and weight of the subject.

### 4.13.1 Methods of Delivery

In one aspect, provided herein are methods of delivering any one or more of a Cas endonuclease (or a functional fragment, functional variant, or domain thereof), a fusion protein; a conjugate; a system (or any one or more component thereof); a nucleic acid molecule; a vector; a reaction mixture; a carrier; and/or pharmaceutical composition to a cell, the method comprising contacting a cell or introducing into a cell a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11), to thereby deliver the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition to the cell. In some embodiments, the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition is contacted to the cell or introduced into the cell in an amount and for a period of time sufficient to deliver the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition to the cell.

In some embodiments, the cell is a eukaryotic cell, e.g., a cell of a multicellular organism, e.g., an animal, e.g., a mammal (e.g., human, swine, bovine) a bird (e.g., poultry, such as chicken, turkey, or duck), or a fish. In some embodiments, the cell is a non-human animal cell (e.g., a laboratory animal, a livestock animal, or a companion animal). In some embodiments, the cell is a stem cell (e.g., a hematopoietic stem cell), a fibroblast, or a T cell. In some embodiments, the cell is a non-dividing cell, e.g., a nondividing fibroblast or non-dividing T cell. In some embodiments, the cell is a eukaryotic cell (e.g., a mammalian cell, an animal cell, a primate cell, a non-human primate cell, a human cell). In some embodiments, the cell is a human cell. In some embodiments, the cell is a plant cell. In some embodiments, the cell is euploid, is not immortalized, is part of a tissue, is part of an organism, is a primary cell, is non-dividing, is haploid (e.g., a germline cell), is a non-cancerous polyploid cell, or is from a subject having a genetic disease.

In some embodiments, the cell is in vitro, ex vivo, in vivo. In some embodiments, the cell is within a subject. In some embodiments, the subject is a mammal, animal, non-human primate, primate, human, or plant. In some embodiments, the subject is a human. In some embodiments, the cell is subsequently administered to a subject (e.g., for a therapeutic application (e.g., described herein (e.g., gene therapy))).

In one aspect, provided herein are methods of delivering any one or more of a Cas endonuclease (or a functional fragment, functional variant, or domain thereof), a fusion protein; a conjugate; a system (or any one or more component thereof); a nucleic acid molecule; a vector; a reaction mixture; a carrier; and/or pharmaceutical composition to a subject, the method comprising administering to the subject a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11), to thereby deliver the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition to the cell. In some embodiments, the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition is administered to the subject in an amount and for a period of time sufficient to deliver the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition to the subject. In some embodiments, the subject is a mammal, animal, non-human primate, primate, human, or plant. In some embodiments, the subject is a human.

### 4.13.2 Methods of Cleaving a Target Nucleic Acid Molecule

In one aspect, provided herein are methods of cleaving a target site in a target nucleic acid (e.g., DNA) molecule (e.g., a double stranded target nucleic acid sequence (e.g., dsDNA, (e.g., genomic dsDNA))), the method comprising contacting the target nucleic acid (e.g., DNA) molecule (e.g., a double stranded target nucleic acid sequence (e.g., dsDNA, (e.g., genomic dsDNA)) with any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11), to thereby cleave the target site in the target nucleic acid (e.g., DNA) molecule (e.g., a double stranded target nucleic acid sequence (e.g., dsDNA, (e.g., genomic dsDNA)). In some embodiments, the method comprises contacting the target nucleic acid (e.g., DNA) molecule (e.g., a double stranded target nucleic acid sequence (e.g., dsDNA, (e.g., genomic dsDNA)) with the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition in an amount and for a period of time sufficient to cleave the target site in the target stranded target nucleic acid sequence (e.g., dsDNA, (e.g., genomic dsDNA))).

In some embodiments, the target nucleic acid molecule is a nucleic acid molecule described herein (see, e.g., § 4.5.1). In some embodiments, the target nucleic acid molecule is a DNA molecule. In some embodiments, the target nucleic acid molecule is a dsDNA molecule. In some embodiments, the target nucleic acid molecule is within a cell (e.g., within the genome (e.g., a gene) of a cell (e.g., a eukaryotic cell) in vitro, ex vivo, or in vivo). In some embodiments, the target nucleic acid molecule is a gene (e.g., within a cell (e.g., a eukaryotic cell) (e.g., within a subject (e.g., a human subject)). In some embodiments, the target nucleic acid molecule is a gene within a cell (e.g., a eukaryotic cell) (e.g., within a subject (e.g., a human subject)). In some embodiments, the target nucleic acid molecule is a gene within a cell (e.g., a eukaryotic cell) within a subject (e.g., a human subject). In some embodiments, the target nucleic acid molecule is genomic DNA or RNA. In some embodiments, the target nucleic acid molecule is within the genome of cell (e.g., a eukaryotic cell) (e.g., within a subject (e.g., a human subject)). In some embodiments, the target nucleic acid molecule is within a cell (e.g., within the genome (e.g., a gene) of a cell (e.g., a eukaryotic cell)) within a subject (e.g., a human subject).

In one aspect, provided herein are a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11) for use in cleaving a target site in a target nucleic acid (e.g., DNA) molecule (e.g., a double stranded target nucleic acid sequence (e.g., dsDNA, (e.g., genomic dsDNA))) in a subject.

In one aspect, provided herein are uses of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11) for cleaving a target site in a target nucleic acid (e.g., DNA) molecule (e.g., a double stranded target nucleic acid sequence (e.g., dsDNA, (e.g., genomic dsDNA))) in a subject.

### 4.13.3 Methods of Editing a Target Nucleic Acid Molecule

In one aspect, provided herein are methods of editing a target site in a target nucleic acid (e.g., DNA) molecule (e.g., a double stranded target nucleic acid sequence (e.g., dsDNA, (e.g., genomic dsDNA))), the method comprising contacting the target nucleic acid (e.g., DNA) molecule (e.g., a double stranded target nucleic acid sequence (e.g., dsDNA, (e.g., genomic dsDNA))) with any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11), to thereby edit the target nucleic acid (e.g., DNA) molecule (e.g., a double stranded target nucleic acid sequence (e.g., dsDNA, (e.g., genomic dsDNA))). In some embodiments, the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition is introduced in an amount and for a period of time sufficient to edit target nucleic acid (e.g., DNA) molecule (e.g., a double stranded target nucleic acid sequence (e.g., dsDNA, (e.g., genomic dsDNA))).

In some embodiments, the edit comprises a substitution, addition, deletion, or inversion of the target site in the target nucleic acid (e.g., DNA) molecule (e.g., a double stranded target nucleic acid sequence (e.g., dsDNA, (e.g., genomic dsDNA))). In some embodiments, the edit comprises an addition, a deletion, or a substitution of one or more nucleotides into/from the target site of the target site in the target nucleic acid (e.g., DNA) molecule (e.g., a double stranded target nucleic acid sequence (e.g., dsDNA, (e.g., genomic dsDNA))). In some embodiments, the edit comprises the addition of one or more nucleotides into the target site in the target nucleic acid (e.g., DNA) molecule (e.g., a double stranded target nucleic acid sequence (e.g., dsDNA, (e.g., genomic dsDNA))). In some embodiments, the addition comprises the addition of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the deletion of one or more nucleotides of the target site in the target nucleic acid (e.g., DNA) molecule (e.g., a double stranded target nucleic acid sequence (e.g., dsDNA, (e.g., genomic dsDNA))). In some embodiments, the deletion comprises the deletion of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the substitution of one or more nucleotides at the target site in the target nucleic acid (e.g., DNA) molecule (e.g., a double stranded target nucleic acid sequence (e.g., dsDNA, (e.g., genomic dsDNA))).

In some embodiments, the target nucleic acid molecule is a nucleic acid molecule described herein (see, e.g., § 4.5.1). In some embodiments, the target nucleic acid molecule is a DNA molecule. In some embodiments, the target nucleic acid molecule is a dsDNA molecule. In some embodiments, the target nucleic acid molecule is within a cell (e.g., within the genome (e.g., a gene) of a cell (e.g., a eukaryotic cell) in vitro, ex vivo, or in vivo). In some embodiments, the target nucleic acid molecule is a gene (e.g., within a cell (e.g., a eukaryotic cell) (e.g., within a subject (e.g., a human subject)). In some embodiments, the target nucleic acid molecule is a gene within a cell (e.g., a eukaryotic cell) (e.g., within a subject (e.g., a human subject)). In some embodiments, the target nucleic acid molecule is a gene within a cell (e.g., a eukaryotic cell) within a subject (e.g., a human subject). In some embodiments, the target nucleic acid molecule is genomic DNA or RNA. In some embodiments, the target nucleic acid molecule is within the genome of cell (e.g., a eukaryotic cell) (e.g., within a subject (e.g., a human subject)). In some embodiments, the target nucleic acid molecule is within a cell (e.g., within the genome (e.g., a gene) of a cell (e.g., a eukaryotic cell)) within a subject (e.g., a human subject).

In one aspect, provided herein are a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11) for use in cleaving a target site in editing target nucleic acid (e.g., DNA) molecule (e.g., a double stranded target nucleic acid sequence (e.g., dsDNA, (e.g., genomic dsDNA))) in a subject.

In one aspect, provided herein are uses of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11) for n cleaving a target site in editing target nucleic acid (e.g., DNA) molecule (e.g., a double stranded target nucleic acid sequence (e.g., dsDNA, (e.g., genomic dsDNA))) in a subject.

### 4.13.3.1 Methods of Editing a Target Nucleic Acid Molecule Utilizing an RT-Based System

In one aspect, provided herein are methods of editing a target site in a target nucleic acid (e.g., DNA) molecule (e.g., a double stranded target nucleic acid sequence (e.g., dsDNA, (e.g., genomic dsDNA))), the method comprising contacting target nucleic acid (e.g., DNA) molecule (e.g., a double stranded target nucleic acid sequence (e.g., dsDNA, (e.g., genomic dsDNA))) with a fusion protein comprising Cas endonuclease (or a functional fragment, functional variant, or domain thereof) described herein (see, e.g., § 4.2) and a reverse transcriptase (e.g., a reverse transcriptase described herein (see, e.g., § 4.3.1.1)) (or a nucleic acid molecule (e.g., a DNA, RNA, nucleic acid molecule) encoding the fusion protein) and a template RNA (e.g., a single template RNA, a plurality of different template RNAs (e.g., a template RNA described herein (see, e.g., § 4.5.2)) (or a nucleic acid molecule (e.g., a DNA nucleic acid molecule) encoding the template RNA); to thereby edit the target nucleic acid (e.g., DNA) molecule (e.g., a double stranded target nucleic acid sequence (e.g., dsDNA, (e.g., genomic dsDNA))). In some embodiments, the fusion protein and the template gRNA are introduced in an amount and for a period of time sufficient to edit the target site in the target nucleic acid (e.g., DNA) molecule (e.g., a double stranded target nucleic acid sequence (e.g., dsDNA, (e.g., genomic dsDNA))).

In some embodiments, the edit comprises a substitution, addition, deletion, or inversion of the target site in the target nucleic acid (e.g., DNA) molecule (e.g., a double stranded target nucleic acid sequence (e.g., dsDNA, (e.g., genomic dsDNA))). In some embodiments, the edit comprises an addition, a deletion, or a substitution of one or more nucleotides into/from the target site of the target site in the target nucleic acid (e.g., DNA) molecule (e.g., a double stranded target nucleic acid sequence (e.g., dsDNA, (e.g., genomic dsDNA))). In some embodiments, the edit comprises the addition of one or more nucleotides into the target site in the target nucleic acid (e.g., DNA) molecule (e.g., a double stranded target nucleic acid sequence (e.g., dsDNA, (e.g., genomic dsDNA))). In some embodiments, the addition comprises the addition of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the deletion of one or more nucleotides of the target site in the target nucleic acid (e.g., DNA) molecule (e.g., a double stranded target nucleic acid sequence (e.g., dsDNA, (e.g., genomic dsDNA))). In some embodiments, the deletion comprises the deletion of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the substitution of one or more nucleotides at the target site in the target nucleic acid (e.g., DNA) molecule (e.g., a double stranded target nucleic acid sequence (e.g., dsDNA, (e.g., genomic dsDNA))).

In some embodiments, the target nucleic acid molecule is a nucleic acid molecule described herein (see, e.g., § 4.5.1). In some embodiments, the target nucleic acid molecule is a DNA molecule. In some embodiments, the target nucleic acid molecule is a dsDNA molecule. In some embodiments, the target nucleic acid molecule is within a cell (e.g., within the genome (e.g., a gene) of a cell (e.g., a eukaryotic cell) in vitro, ex vivo, or in vivo). In some embodiments, the target nucleic acid molecule is a gene (e.g., within a cell (e.g., a eukaryotic cell) (e.g., within a subject (e.g., a human subject)). In some embodiments, the target nucleic acid molecule is a gene within a cell (e.g., a eukaryotic cell) (e.g., within a subject (e.g., a human subject)). In some embodiments, the target nucleic acid molecule is a gene within a cell (e.g., a eukaryotic cell) within a subject (e.g., a human subject). In some embodiments, the target nucleic acid molecule is genomic DNA or RNA. In some embodiments, the target nucleic acid molecule is within the genome of cell (e.g., a eukaryotic cell) (e.g., within a subject (e.g., a human subject)). In some embodiments, the target nucleic acid molecule is within a cell (e.g., within the genome (e.g., a gene) of a cell (e.g., a eukaryotic cell)) within a subject (e.g., a human subject).

In one aspect, provided herein are methods of editing a target site in a target nucleic acid (e.g., DNA) molecule (e.g., a double stranded target nucleic acid sequence (e.g., dsDNA, (e.g., genomic dsDNA))), the method comprising contacting the target nucleic acid (e.g., DNA) molecule (e.g., a double stranded target nucleic acid sequence (e.g., dsDNA, (e.g., genomic dsDNA))) with a system described in § 4.5.5.2, to thereby edit the target nucleic acid (e.g., DNA) molecule (e.g., a double stranded target nucleic acid sequence (e.g., dsDNA, (e.g., genomic dsDNA))). In some embodiments, the system is introduced in an amount and for a period of time sufficient to edit the target site in the target nucleic acid (e.g., DNA) molecule (e.g., a double stranded target nucleic acid sequence (e.g., dsDNA, (e.g., genomic dsDNA))).

In some embodiments, the edit comprises a substitution, addition, deletion, or inversion of the target site in the target nucleic acid (e.g., DNA) molecule (e.g., a double stranded target nucleic acid sequence (e.g., dsDNA, (e.g., genomic dsDNA))). In some embodiments, the edit comprises an addition, a deletion, or a substitution of one or more nucleotides into/from the target site of the target site in the target nucleic acid (e.g., DNA) molecule (e.g., a double stranded target nucleic acid sequence (e.g., dsDNA, (e.g., genomic dsDNA))). In some embodiments, the edit comprises the addition of one or more nucleotides into the target site in the target nucleic acid (e.g., DNA) molecule (e.g., a double stranded target nucleic acid sequence (e.g., dsDNA, (e.g., genomic dsDNA))). In some embodiments, the addition comprises the addition of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the deletion of one or more nucleotides of the target site in the target nucleic acid (e.g., DNA) molecule (e.g., a double stranded target nucleic acid sequence (e.g., dsDNA, (e.g., genomic dsDNA))). In some embodiments, the deletion comprises the deletion of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the substitution of one or more nucleotides at the target site in the target nucleic acid (e.g., DNA) molecule (e.g., a double stranded target nucleic acid sequence (e.g., dsDNA, (e.g., genomic dsDNA))).

In some embodiments, the target nucleic acid molecule is a nucleic acid molecule described herein (see, e.g., § 4.5.1). In some embodiments, the target nucleic acid molecule is a DNA molecule. In some embodiments, the target nucleic acid molecule is a dsDNA molecule. In some embodiments, the target nucleic acid molecule is within a cell (e.g., within the genome (e.g., a gene) of a cell (e.g., a eukaryotic cell) in vitro, ex vivo, or in vivo). In some embodiments, the target nucleic acid molecule is a gene (e.g., within a cell (e.g., a eukaryotic cell) (e.g., within a subject (e.g., a human subject)). In some embodiments, the target nucleic acid molecule is a gene within a cell (e.g., a eukaryotic cell) (e.g., within a subject (e.g., a human subject)). In some embodiments, the target nucleic acid molecule is a gene within a cell (e.g., a eukaryotic cell) within a subject (e.g., a human subject). In some embodiments, the target nucleic acid molecule is genomic DNA or RNA. In some embodiments, the target nucleic acid molecule is within the genome of cell (e.g., a eukaryotic cell) (e.g., within a subject (e.g., a human subject)). In some embodiments, the target nucleic acid molecule is within a cell (e.g., within the genome (e.g., a gene) of a cell (e.g., a eukaryotic cell)) within a subject (e.g., a human subject).

### 4.13.3.2 Methods of Editing a Target Nucleic Acid Molecule Utilizing an HDR-Based System

In one aspect, provided herein are methods of editing a target site in a target nucleic acid (e.g., DNA) molecule (e.g., a double stranded target nucleic acid sequence (e.g., dsDNA, (e.g., genomic dsDNA))), the method comprising contacting the target nucleic acid (e.g., DNA) molecule (e.g., a double stranded target nucleic acid sequence (e.g., dsDNA, (e.g., genomic dsDNA))) with a system described in § 4.5.5.1, to thereby edit target nucleic acid (e.g., DNA) molecule (e.g., a double stranded target nucleic acid sequence (e.g., dsDNA, (e.g., genomic dsDNA))). In some embodiments, the system is introduced in an amount and for a period of time sufficient to edit the target site in the target nucleic acid (e.g., DNA) molecule (e.g., a double stranded target nucleic acid sequence (e.g., dsDNA, (e.g., genomic dsDNA))).

In some embodiments, the edit comprises a substitution, addition, deletion, or inversion of the target site in the target nucleic acid (e.g., DNA) molecule (e.g., a double stranded target nucleic acid sequence (e.g., dsDNA, (e.g., genomic dsDNA))). In some embodiments, the edit comprises an addition, a deletion, or a substitution of one or more nucleotides into/from the target site of the target site in the target nucleic acid (e.g., DNA) molecule (e.g., a double stranded target nucleic acid sequence (e.g., dsDNA, (e.g., genomic dsDNA))). In some embodiments, the edit comprises the addition of one or more nucleotides into the target site in the target nucleic acid (e.g., DNA) molecule (e.g., a double stranded target nucleic acid sequence (e.g., dsDNA, (e.g., genomic dsDNA))). In some embodiments, the addition comprises the addition of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the deletion of one or more nucleotides of the target site in the target nucleic acid (e.g., DNA) molecule (e.g., a double stranded target nucleic acid sequence (e.g., dsDNA, (e.g., genomic dsDNA))). In some embodiments, the deletion comprises the deletion of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the substitution of one or more nucleotides at the target site in the target nucleic acid (e.g., DNA) molecule (e.g., a double stranded target nucleic acid sequence (e.g., dsDNA, (e.g., genomic dsDNA))).

In some embodiments, the target nucleic acid molecule is a nucleic acid molecule described herein (see, e.g., § 4.5.1). In some embodiments, the target nucleic acid molecule is a DNA molecule. In some embodiments, the target nucleic acid molecule is a dsDNA molecule. In some embodiments, the target nucleic acid molecule is within a cell (e.g., within the genome (e.g., a gene) of a cell (e.g., a eukaryotic cell) in vitro, ex vivo, or in vivo). In some embodiments, the target nucleic acid molecule is a gene (e.g., within a cell (e.g., a eukaryotic cell) (e.g., within a subject (e.g., a human subject)). In some embodiments, the target nucleic acid molecule is a gene within a cell (e.g., a eukaryotic cell) (e.g., within a subject (e.g., a human subject)). In some embodiments, the target nucleic acid molecule is a gene within a cell (e.g., a eukaryotic cell) within a subject (e.g., a human subject). In some embodiments, the target nucleic acid molecule is genomic DNA or RNA. In some embodiments, the target nucleic acid molecule is within the genome of cell (e.g., a eukaryotic cell) (e.g., within a subject (e.g., a human subject)). In some embodiments, the target nucleic acid molecule is within a cell (e.g., within the genome (e.g., a gene) of a cell (e.g., a eukaryotic cell)) within a subject (e.g., a human subject).

### 4.13.3.3 Methods of Editing a Target Nucleic Acid Molecule Utilizing a Nucleobase Editor-Based System

In one aspect, provided herein are methods of editing a target site in a target nucleic acid (e.g., DNA) molecule (e.g., a double stranded target nucleic acid sequence (e.g., dsDNA, (e.g., genomic dsDNA))), the method comprising contacting the target nucleic acid (e.g., DNA) molecule (e.g., a double stranded target nucleic acid sequence (e.g., dsDNA, (e.g., genomic dsDNA))) with a system described in § 4.5.5.3, to thereby edit the target nucleic acid (e.g., DNA) molecule (e.g., a double stranded target nucleic acid sequence (e.g., dsDNA, (e.g., genomic dsDNA))). In some embodiments, the system is introduced in an amount and for a period of time sufficient to edit the target site in the target nucleic acid (e.g., DNA) molecule (e.g., a double stranded target nucleic acid sequence (e.g., dsDNA, (e.g., genomic dsDNA))).

In some embodiments, the edit comprises a substitution, addition, deletion, or inversion of the target site in the target nucleic acid (e.g., DNA) molecule (e.g., a double stranded target nucleic acid sequence (e.g., dsDNA, (e.g., genomic dsDNA))). In some embodiments, the edit comprises an addition, a deletion, or a substitution of one or more nucleotides into/from the target site of the target site in the target nucleic acid (e.g., DNA) molecule (e.g., a double stranded target nucleic acid sequence (e.g., dsDNA, (e.g., genomic dsDNA))). In some embodiments, the edit comprises the addition of one or more nucleotides into the target site in the target nucleic acid (e.g., DNA) molecule (e.g., a double stranded target nucleic acid sequence (e.g., dsDNA, (e.g., genomic dsDNA))). In some embodiments, the addition comprises the addition of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the deletion of one or more nucleotides of the target site in the target nucleic acid (e.g., DNA) molecule (e.g., a double stranded target nucleic acid sequence (e.g., dsDNA, (e.g., genomic dsDNA))). In some embodiments, the deletion comprises the deletion of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the substitution of one or more nucleotides at the target site in the target nucleic acid (e.g., DNA) molecule (e.g., a double stranded target nucleic acid sequence (e.g., dsDNA, (e.g., genomic dsDNA))).

In some embodiments, the target nucleic acid molecule is a nucleic acid molecule described herein (see, e.g., § 4.5.1). In some embodiments, the target nucleic acid molecule is a DNA molecule. In some embodiments, the target nucleic acid molecule is a dsDNA molecule. In some embodiments, the target nucleic acid molecule is within a cell (e.g., within the genome (e.g., a gene) of a cell (e.g., a eukaryotic cell) in vitro, ex vivo, or in vivo). In some embodiments, the target nucleic acid molecule is a gene (e.g., within a cell (e.g., a eukaryotic cell) (e.g., within a subject (e.g., a human subject)). In some embodiments, the target nucleic acid molecule is a gene within a cell (e.g., a eukaryotic cell) (e.g., within a subject (e.g., a human subject)). In some embodiments, the target nucleic acid molecule is a gene within a cell (e.g., a eukaryotic cell) within a subject (e.g., a human subject). In some embodiments, the target nucleic acid molecule is genomic DNA or RNA. In some embodiments, the target nucleic acid molecule is within the genome of cell (e.g., a eukaryotic cell) (e.g., within a subject (e.g., a human subject)). In some embodiments, the target nucleic acid molecule is within a cell (e.g., within the genome (e.g., a gene) of a cell (e.g., a eukaryotic cell)) within a subject (e.g., a human subject).

Standard methods of assessing the editing of a target nucleic acid molecule (e.g., in a cell) are known in the art and described herein. See, e.g., §§ 4.5.4, 5.2. See also, e.g., Glaser A, McColl B, Vadolas J. GFP to BFP Conversion: A Versatile Assay for the Quantification of CRISPR/Cas9-mediated Genome Editing [published correction appears in Mol Ther Nucleic Acids. 2016 Sep. 13; 5(9):e360]. Mol Ther Nucleic Acids. 2016; 5(7):e334. Published 2016 Jul. 12. doi:10.1038/mtna.2016.48, the entire contents of which are incorporated by reference herein for all purposes.

### 4.13.4 Methods of Treating, Ameliorating, or Preventing a Disease

In one aspect, provided herein are methods of treating, ameliorating, or preventing a disease in a subject (e.g., a human subject) in need thereof, the method comprising administering to the subject any one or more of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11), to thereby treat, ameliorate, or prevent the disease in the subject (e.g., the human subject). In some embodiments, the endonuclease (or a functional fragment, functional variant, or domain thereof), the fusion protein; the conjugate; the system (or any one or more component thereof); the nucleic acid molecule; the vector; the reaction mixture; the carrier; and/or the pharmaceutical composition is introduced in an amount and for a period of time sufficient to treat, ameliorate, or prevent the disease in the subject (e.g., the human subject).

Exemplary diseases include, but are not limited to, e.g., genetic disorders; cancer (e.g., cancers associated with genetic variations (e.g., point mutations, alternatively splicing, gene duplications, etc.); diseases associated with overexpression of RNA, toxic RNA, and/or mutated RNA (e.g., splicing defects or truncations); and infections (e.g., a viral, bacterial, parasitic, or protozoal infection). In some embodiments, the disease is a genetic disorder.

In some embodiments, the subject is a mammal, animal, primate, non-human primate, or human. In some embodiments, the subject is a human.

In some embodiments, the disease is associated with a genetic defect. In some embodiments, wherein a gRNA and a Cas endonuclease (e.g., of a system described herein) are administered to the subject, the gRNA is capable of targeting the endonuclease to the site of the genetic defect. In some embodiments, the genetic defect comprises a duplication of a gene, deletion of a gene, or a mutation of a gene. In some embodiments, the administration results in the correction of the genetic defect. In some embodiments, the genetic defect comprises a mutation in a gene. In some embodiments, the mutation is a substitution, addition, deletion, or inversion. In some embodiments, the genetic defect comprises a mutation in a gene and the administration corrects the mutation (e.g., substitution, addition, deletion, or inversion) in the gene. In some embodiments, the administration results in the replacement of the mutated nucleotide sequence with the corresponding wild type nucleotide sequence. In some embodiments, the genetic defect is a deletion of a gene (or a portion thereof). In some embodiments, the genetic defect is a deletion of part or an entire gene and the administration inserts the deleted gene (or portion thereof). In some embodiments, the genetic defect is the duplication of a gene (or a portion thereof). In some embodiments, the genetic defect is the duplication of a gene (or a portion thereof), and the administration deletes the duplicated gene (or the portion thereof).

In some embodiments, the administration results in the editing of a target site in a target nucleic acid (e.g., DNA) molecule (e.g., a double stranded target nucleic acid sequence (e.g., dsDNA, (e.g., genomic dsDNA))). In some embodiments, the edit comprises a substitution, addition, deletion, or inversion of the target site in the target nucleic acid (e.g., DNA) molecule (e.g., a double stranded target nucleic acid sequence (e.g., dsDNA, (e.g., genomic dsDNA))). In some embodiments, the edit comprises an addition, a deletion, or a substitution of one or more nucleotides into/from the target site of the target site in the target nucleic acid (e.g., DNA) molecule (e.g., a double stranded target nucleic acid sequence (e.g., dsDNA, (e.g., genomic dsDNA))). In some embodiments, the edit comprises the addition of one or more nucleotides into the target site in the target nucleic acid (e.g., DNA) molecule (e.g., a double stranded target nucleic acid sequence (e.g., dsDNA, (e.g., genomic dsDNA))). In some embodiments, the addition comprises the addition of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the deletion of one or more nucleotides of the target site in the target nucleic acid (e.g., DNA) molecule (e.g., a double stranded target nucleic acid sequence (e.g., dsDNA, (e.g., genomic dsDNA))). In some embodiments, the deletion comprises the deletion of from about 1-500, 1-3200, 1-300, 1-200, 1-100, 1-90, 1-80, 1-70, 1-60, 1-50, 1-320, 1-30, 1-20, 1-10, 1-9, 1-8, 1-7, 1-6, 1-5, 1-4, 1-3, or 1-2 nucleotides. In some embodiments, the edit comprises the substitution of one or more nucleotides at the target site in the target nucleic acid (e.g., DNA) molecule (e.g., a double stranded target nucleic acid sequence (e.g., dsDNA, (e.g., genomic dsDNA))).

In one aspect, provided herein are a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11) for the manufacture of a medicament.

In one aspect, provided herein are uses of a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11) for the manufacture of a medicament for the treatment of a disease in a subject in need thereof (e.g., a disease is associated with a genetic defect).

In one aspect, provided herein are a Cas endonuclease (or a functional fragment, functional variant, or domain thereof) described herein (see, e.g., § 4.2), a fusion protein described herein (see, e.g., § 4.3); a conjugate described herein (see, e.g., § 4.3); a system described herein (see, e.g., § 4.5) (or any one or more component thereof); a nucleic acid molecule described herein (see, e.g., § 4.6); a vector described herein (see, e.g., § 4.7); a reaction mixture described herein (see, e.g., § 4.10); a carrier described herein (see, e.g., § 4.8); and/or pharmaceutical composition described herein (see, e.g., § 4.11) for the manufacture of a medicament for the treatment of a disease in a subject in need thereof (e.g., a disease is associated with a genetic defect).

### 5. EXAMPLES

### Table of Contents

5.1 Example 1. Cas Endonuclease Generation and Expression.
5.2 Example 2. Nucleic Acid Editing Activity of Cas Endonucleases.
5.3 Example 3. Nucleic Acid Editing Activity of Exemplary Cas Endonucleases.
5.4 Example 4. Nucleic Acid Editing Activity of Cas Endonucleases in HBB K562 cells.

### 5.1 Example 1. Cas Endonuclease Generation and Expression

Novel endonucleases 41-360 (CasEnds 41-360) (set forth in Table 1 and SEQ ID NOS: 1-320) were identified by the inventors through a process of rational design, computer-aided design, molecular modeling and binding and functional screening of over 690 candidate library sequences.

The endonucleases were expressed using standard methods known in the art. A reference Cas endonuclease (Cas9 Nickase) was also expressed according to the methods described above. The amino acid sequence of the reference Cas endonuclease is set forth in Table 5 and in SEQ ID NO: 321.

TABLE 5
The Amino Acid Sequence of Reference Cas Endonuclease.

| | | SEQ |
|---|---|---|
| Description | Amino Acid Sequence | ID NO |
| Reference Cas | MDKKYSIGLDIGTNSVGWAVITDEYKVPSKKFKVLGNTDRHSIKKNLIGA | 321 |
| Endonuclease | LLFDSGETAEATRLKRTARRRYTRRKNRICYLQEIFSNEMAKVDDSFFHR | |
| Cas9 Nickase | LEESFLVEEDKKHERHPIFGNIVDEVAYHEKYPTIYHLRKKLVDSTDKAD | |
| N863A | LRLIYLALAHMIKFRGHFLIEGDLNPDNSDVDKLFIQLVQTYNQLFEENP | |
| | INASGVDAKAILSARLSKSRRLENLIAQLPGEKKNGLFGNLIALSLGLTP | |
| | NFKSNFDLAEDAKLQLSKDTYDDDLDNLLAQIGDQYADLFLAAKNLSDAI | |
| | LLSDILRVNTEITKAPLSASMIKRYDEHHQDLTLLKALVRQQLPEKYKEI | |
| | FFDQSKNGYAGYIDGGASQEEFYKFIKPILEKMDGTEELLVKLNREDLLR | |
| | KQRTFDNGSIPHQIHLGELHAILRRQEDFYPFLKDNREKIEKILTFRIPY | |
| | YVGPLARGNSRFAWMTRKSEETITPWNFEEVVDKGASAQSFIERMINFDK | |
| | NLPNEKVLPKHSLLYEYFTVYNELTKVKYVTEGMRKPAFLSGEQKKAIVD | |
| | LLFKTNRKVTVKQLKEDYFKKIECFDSVEISGVEDRFNASLGTYHDLLKI | |
| | IKDKDFLDNEENEDILEDIVLTLTLFEDREMIEERLKTYAHLEDDKVMKQ | |
| | LKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKV | |
| | MGRHKPENIVIEMARENQTTQKGQKNSRERMKRIEEGIKELGSQILKEHP | |
| | VENTQLQNEKLYLYYLQNGRDMYVDQELDINRLSDYDVDHIVPQSFLKDD | |
| | SIDNKVLTRSDKARGKSDNVPSEEVVKKMKNYWRQLLNAKLITQRKFDNL | |
| | TKAERGGLSELDKAGFIKRQLVETRQITKHVAQILDSRMNTKYDENDKLI | |
| | REVKVITLKSKLVSDFRKDFQFYKVREINNYHHAHDAYLNAVVGTALIKK | |
| | YPKLESEFVYGDYKVYDVRKMIAKSEQEIGKATAKYFFYSNIMNFFKTEI | |
| | TLANGEIRKRPLIETNGETGEIVWDKGRDFATVRKVLSMPQVNIVKKTEV | |
| | QTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPK | |
| | YSLFELENGRKRMLASAGELQKGNELALPSKYVNFLYLASHYEKLKGSPE | |
| | DNEQKQLFVEQHKHYLDEIIEQISEFSKRVILADANLDKVLSAYNKHRDK | |
| | PIREQAENIIHLFTLTNLGAPAAFKYFDTTIDRKRYTSTKEVLDATLIHQ | |
| | SITGLYETRIDLSQLGGD | |

### 5.2 Example 2. Nucleic Acid Editing Activity of Cas Endonucleases

The ability of the candidate endonucleases, including endonucleases 41-360 (CasEnds 41-360) (set forth in Table 1 and SEQ ID NOS: 1-320), to mediate target nucleic acid editing was assessed utilizing a blue fluorescent protein (BFP) to green fluorescent protein (GFP) conversion assay, wherein programmed nucleotide editing of the BFP gene was measured by the expression of GFP (signifying the conversion of GFP to BFP via the programmed nucleotide edit in the BFP gene). The conversion assay was conducted utilizing a reverse transcriptase-based system (as described herein) comprising a template RNA (designed to convert BFP to GFP) and a fusion protein comprising a retroviral reverse transcriptase and the individual subject Cas endonuclease.

The nucleotide sequence of the template RNA is set forth in Table 6.

TABLE 6
The Nucleotide Sequence of Template RNA.

| | | SEQ |
|---|---|---|
| Description | Nucleotide Sequence | ID NO |
| Template | GCCGAAGCACTGCACGCCGTGTTTTAGAGCTAGAAATAGCAAGTTAAAAT | 322 |
| RNA | AAGGCTAGTCCGTTATCAACTTGAAAAAGTGGCACCGAGTCGGTGCACCC | |
| | TGACGTACGGCGTGCAGTGCTT | |

The amino acid sequence of the base portion of the fusion protein (without the individual subject Cas endonuclease) is set forth in Table 7.

TABLE 7
The Amino Acid Sequence of the Fusion Protein.

| | | SEQ |
|---|---|---|
| Description | Nucleotide Sequence | ID NO |
| Fusion | MPAAKRVKLDGGDKKYSIGLDIGTNSVGWAVITDEYKVPSKKFKVLGNTD | 323 |
| Protein | RHSIKKNLIGALLFDSGETAEATRLKRTARRRYTRRKNRICYLQEIFSNE | |
| | MAKVDDSFFHRLEESFLVEEDKKHERHPIFGNIVDEVAYHEKYPTIYHLR | |
| | KKLVDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGLFG | |
| | NLIALSLGLTPNFKSNFDLAEDAKLQLSKDTYDDDLDNLLAQIGDQYADL | |
| | FLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEEL | |
| | LVKLNREDLLRKQRTFDNGSIPHQIHLGELHAILRRQEDFYPFLKDNREK | |
| | IEKILTFRIPYYVGPLARGNSRFAWMTRKSEETITPWNFEEVVDKGASAQ | |
| | SFIERMTNFDKNLPNEKVLPKHSLLYEYFTVYNELTKVKYVTEGMRKPAF | |
| | LSGEQKKAIVDLLFKTNRKVTVKQLKEDYFKKIECFDSVEISGVEDRFNA | |
| | SLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFA | |
| | NRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVD | |
| | HIVPQSFLKDDSIDNKVLTRSDKARGKSDNVPSEEVVKKMKNYWRQLLNA | |
| | KLITQRKFDNLTKAERGGLSELDKAGFIKRQLVETRQITKHVAQILDSRM | |
| | NTKYDENDKLIREVKVITLKSKLVSDFRKDFQFYKVREINNYHHAHDAYL | |
| | NAVVGTALIKKYPKLESEFVYGDYKVYDVRKMIAKSEQEIGKATAKYFFY | |
| | SNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFDSPTV | |
| | AYSVLVVAKVEKGKSKKLKSVKELLGITIMERSSFEKNPIDFLEAKGYKE | |
| | VKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDK | |
| | VLSAYNKHRDKPIREQAENIIHLFTLTNLGAPAAFKYFDTTIDRKRYTST | |
| | KEVLDATLIHQSITGLYETRIDLSQLGGDGGAEAAAKEAAAKEAAAKEAA | |
| | AKALEAEAAAKEAAAKEAAAKEAAAKAGGTAPLEEEYRLFLEAPIQNVTL | |
| | LEQWKREIPKVWAEINPPGLASTQAPIHVQLLSTALPVRVRQYPITLEAK | |
| | RSLRETIRKFRAAGILRPVHSPWNTPLLPVRKSGTSEYRMVQDLREVNKR | |
| | VETIHPTVPNPYTLLSLLPPDRIWYSVLDLKDAFFCIPLAPESQLIFAFE | |
| | WADAEEGESGQLTWTRLPQGFKNSPTLFNEALNRDLQGFRLDHPSVSLLQ | |
| | YVDDLLIAADTQAACLSATRDLLMTLAELGYRVSGKKAQLCQEEVTYLGF | |
| | KIHKGSRSLSNSRTQAILQIPVPKTKRQVREFLGKIGYCRLFIPGFAELA | |
| | QPLYAATRPGNDPLVWGEKEEEAFQSLKLALTQPPALALPSLDKPFQLFV | |
| | EETSGAAKGVLTQALGPWKRPVAYLSKRLDPVAAGWPRCLRAIAAAALLT | |
| | REASKLIFGQDIEITSSHNLESLLRSPPDKWLTNARITQYQVLLLDPPRV | |
| | RFKQTAALNPATLLPETDDTLPIHHCLDTLDSLTSTRPDLTDQPLAQAEA | |
| | TLFTDGSSYIRDGKRYAGAAVVTLDSVIWAEPLPIGTSAQKAELIALTKA | |
| | LEWSKDKSVNIYTDSRYAFATLHVHGMIYRERGWLTAGGKAIKNAPEILA | |
| | LLTAVWLPKRVAVMHCKGHQKDDAPTSTGNRRADEVAREVAIRPLSTQAT | |
| | ISAGKRTADGSEFEKRTADGSEFESPKKKAKVE | |

Briefly, 200 ng of plasmid DNA encoding the subject fusion protein (containing one of the subject CasEnds) and 200 ng of template RNA (in plasmid format) were added to 25 μL SF buffer containing 250,000 HEK293T BFP-expressing cells. Nucleofection was mediated utilizing program DS-150. The day of nucleofection was marked as day 0. At day 4, the cells were harvested and analyzed by flow cytometry to assess the level of BFP and GFP expression. Cells having GFP signal were defined as having undergone a successful editing event, and the percent of cells that were GFP+ on day 4 was used to determine the performance of each Cas endonuclease.

Of the over 690 potential endonucleases generated, around half had editing activity, with some exhibiting even higher editing activity than the editing activity of a reference Cas endonuclease (SEQ ID NO: 321). See, Table 8 below.

TABLE 8
The Relative Editing Activity of Cas Endonucleases.

| Description | SEQ ID NO | Relative Editing Activity |
|---|---|---|
| CasEnd-41 | 1 | +++ |
| CasEnd-42 | 2 | +++ |
| CasEnd-43 | 3 | +++ |
| CasEnd-44 | 4 | +++ |
| CasEnd-45 | 5 | +++ |
| CasEnd-46 | 6 | +++ |
| CasEnd-47 | 7 | +++ |
| CasEnd-48 | 8 | +++ |
| CasEnd-49 | 9 | +++ |
| CasEnd-50 | 10 | +++ |
| CasEnd-51 | 11 | +++ |
| CasEnd-52 | 12 | +++ |
| CasEnd-53 | 13 | +++ |
| CasEnd-54 | 14 | +++ |
| CasEnd-55 | 15 | +++ |
| CasEnd-56 | 16 | +++ |
| CasEnd-57 | 17 | +++ |
| CasEnd-58 | 18 | +++ |
| CasEnd-59 | 19 | +++ |
| CasEnd-60 | 20 | +++ |
| CasEnd-61 | 21 | +++ |
| CasEnd-62 | 22 | ++ |
| CasEnd-63 | 23 | ++ |
| CasEnd-64 | 24 | ++ |
| CasEnd-65 | 25 | ++ |
| CasEnd-66 | 26 | ++ |
| CasEnd-67 | 27 | ++ |
| CasEnd-68 | 28 | ++ |
| CasEnd-69 | 29 | ++ |
| CasEnd-70 | 30 | ++ |
| CasEnd-71 | 31 | ++ |
| CasEnd-72 | 32 | ++ |
| CasEnd-73 | 33 | ++ |
| CasEnd-74 | 34 | ++ |
| CasEnd-75 | 35 | ++ |
| CasEnd-76 | 36 | ++ |
| CasEnd-77 | 37 | ++ |
| CasEnd-78 | 38 | ++ |
| CasEnd-79 | 39 | ++ |
| CasEnd-80 | 40 | ++ |
| CasEnd-81 | 41 | ++ |
| CasEnd-82 | 42 | ++ |
| CasEnd-83 | 43 | ++ |
| CasEnd-84 | 44 | ++ |
| CasEnd-85 | 45 | ++ |
| CasEnd-86 | 46 | ++ |
| CasEnd-87 | 47 | ++ |
| CasEnd-88 | 48 | ++ |
| CasEnd-89 | 49 | ++ |
| CasEnd-90 | 50 | ++ |
| CasEnd-91 | 51 | ++ |
| CasEnd-92 | 52 | ++ |
| CasEnd-93 | 53 | ++ |
| CasEnd-94 | 54 | ++ |
| CasEnd-95 | 55 | ++ |
| CasEnd-96 | 56 | ++ |
| CasEnd-97 | 57 | ++ |
| CasEnd-98 | 58 | ++ |
| CasEnd-99 | 59 | ++ |
| CasEnd-100 | 60 | ++ |
| CasEnd-101 | 61 | ++ |
| CasEnd-102 | 62 | ++ |
| CasEnd-103 | 63 | ++ |
| CasEnd-104 | 64 | ++ |
| CasEnd-105 | 65 | ++ |
| CasEnd-106 | 66 | ++ |
| CasEnd-107 | 67 | ++ |
| CasEnd-108 | 68 | ++ |
| CasEnd-109 | 69 | ++ |
| CasEnd-110 | 70 | ++ |
| CasEnd-111 | 71 | ++ |
| CasEnd-112 | 72 | ++ |
| CasEnd-113 | 73 | ++ |
| CasEnd-114 | 74 | ++ |
| CasEnd-115 | 75 | ++ |
| CasEnd-116 | 76 | ++ |
| CasEnd-117 | 77 | ++ |
| CasEnd-118 | 78 | ++ |
| CasEnd-119 | 79 | ++ |
| CasEnd-120 | 80 | ++ |
| CasEnd-121 | 81 | ++ |
| CasEnd-122 | 82 | ++ |
| CasEnd-123 | 83 | ++ |
| CasEnd-124 | 84 | ++ |
| CasEnd-125 | 85 | ++ |
| CasEnd-126 | 86 | ++ |
| CasEnd-127 | 87 | ++ |
| CasEnd-128 | 88 | ++ |
| CasEnd-129 | 89 | ++ |
| CasEnd-130 | 90 | ++ |
| CasEnd-131 | 91 | ++ |
| CasEnd-132 | 92 | ++ |
| CasEnd-133 | 93 | ++ |
| CasEnd-134 | 94 | ++ |
| CasEnd-135 | 95 | ++ |
| CasEnd-136 | 96 | ++ |
| CasEnd-137 | 97 | ++ |
| CasEnd-138 | 98 | ++ |
| CasEnd-139 | 99 | ++ |
| CasEnd-140 | 100 | ++ |
| CasEnd-141 | 101 | ++ |
| CasEnd-142 | 102 | ++ |
| CasEnd-143 | 103 | ++ |
| CasEnd-144 | 104 | ++ |
| CasEnd-145 | 105 | ++ |
| CasEnd-146 | 106 | ++ |
| CasEnd-147 | 107 | ++ |
| CasEnd-148 | 108 | ++ |
| CasEnd-149 | 109 | ++ |
| CasEnd-150 | 110 | ++ |
| CasEnd-151 | 111 | ++ |
| CasEnd-152 | 112 | ++ |
| CasEnd-153 | 113 | ++ |
| CasEnd-154 | 114 | ++ |
| CasEnd-155 | 115 | ++ |
| CasEnd-156 | 116 | ++ |
| CasEnd-157 | 117 | + |
| CasEnd-158 | 118 | + |
| CasEnd-159 | 119 | + |
| CasEnd-160 | 120 | + |
| CasEnd-161 | 121 | + |
| CasEnd-162 | 122 | + |
| CasEnd-163 | 123 | + |
| CasEnd-164 | 124 | + |
| CasEnd-165 | 125 | + |
| CasEnd-166 | 126 | + |
| CasEnd-167 | 127 | + |
| CasEnd-168 | 128 | + |
| CasEnd-169 | 129 | + |
| CasEnd-170 | 130 | + |
| CasEnd-171 | 131 | + |
| CasEnd-172 | 132 | + |
| CasEnd-173 | 133 | + |
| CasEnd-174 | 134 | + |
| CasEnd-175 | 135 | + |
| CasEnd-176 | 136 | + |
| CasEnd-177 | 137 | + |
| CasEnd-178 | 138 | + |
| CasEnd-179 | 139 | + |
| CasEnd-180 | 140 | + |
| CasEnd-181 | 141 | + |
| CasEnd-182 | 142 | + |
| CasEnd-183 | 143 | + |
| CasEnd-184 | 144 | + |
| CasEnd-185 | 145 | + |
| CasEnd-186 | 146 | + |
| CasEnd-187 | 147 | + |
| CasEnd-188 | 148 | + |
| CasEnd-189 | 149 | + |
| CasEnd-190 | 150 | + |
| CasEnd-191 | 151 | + |
| CasEnd-192 | 152 | + |
| CasEnd-193 | 153 | + |
| CasEnd-194 | 154 | + |
| CasEnd-195 | 155 | + |
| CasEnd-196 | 156 | + |
| CasEnd-197 | 157 | + |
| CasEnd-198 | 158 | + |
| CasEnd-199 | 159 | + |
| CasEnd-200 | 160 | + |
| CasEnd-201 | 161 | + |
| CasEnd-202 | 162 | + |
| CasEnd-203 | 163 | + |
| CasEnd-204 | 164 | + |
| CasEnd-205 | 165 | + |
| CasEnd-206 | 166 | + |
| CasEnd-207 | 167 | + |
| CasEnd-208 | 168 | + |
| CasEnd-209 | 169 | + |
| CasEnd-210 | 170 | + |
| CasEnd-211 | 171 | + |
| CasEnd-212 | 172 | + |
| CasEnd-213 | 173 | + |
| CasEnd-214 | 174 | + |
| CasEnd-215 | 175 | + |
| CasEnd-216 | 176 | + |
| CasEnd-217 | 177 | + |
| CasEnd-218 | 178 | + |
| CasEnd-219 | 179 | + |
| CasEnd-220 | 180 | + |
| CasEnd-221 | 181 | + |
| CasEnd-222 | 182 | + |
| CasEnd-223 | 183 | + |
| CasEnd-224 | 184 | + |
| CasEnd-225 | 185 | + |
| CasEnd-226 | 186 | + |
| CasEnd-227 | 187 | + |
| CasEnd-228 | 188 | + |
| CasEnd-229 | 189 | + |
| CasEnd-230 | 190 | + |
| CasEnd-231 | 191 | + |
| CasEnd-232 | 192 | + |
| CasEnd-233 | 193 | + |
| CasEnd-234 | 194 | + |
| CasEnd-235 | 195 | + |
| CasEnd-236 | 196 | + |
| CasEnd-237 | 197 | + |
| CasEnd-238 | 198 | + |
| CasEnd-239 | 199 | + |
| CasEnd-240 | 200 | + |
| CasEnd-241 | 201 | + |
| CasEnd-242 | 202 | + |
| CasEnd-243 | 203 | + |
| CasEnd-244 | 204 | + |
| CasEnd-245 | 205 | + |
| CasEnd-246 | 206 | + |
| CasEnd-247 | 207 | + |
| CasEnd-248 | 208 | + |
| CasEnd-249 | 209 | + |
| CasEnd-250 | 210 | + |
| CasEnd-251 | 211 | + |
| CasEnd-252 | 212 | + |
| CasEnd-253 | 213 | + |
| CasEnd-254 | 214 | + |
| CasEnd-255 | 215 | + |
| CasEnd-256 | 216 | + |
| CasEnd-257 | 217 | + |
| CasEnd-258 | 218 | + |
| CasEnd-259 | 219 | + |
| CasEnd-260 | 220 | + |
| CasEnd-261 | 221 | + |
| CasEnd-262 | 222 | + |
| CasEnd-263 | 223 | + |
| CasEnd-264 | 224 | + |
| CasEnd-265 | 225 | + |
| CasEnd-266 | 226 | + |
| CasEnd-267 | 227 | + |
| CasEnd-268 | 228 | + |
| CasEnd-269 | 229 | + |
| CasEnd-270 | 230 | + |
| CasEnd-271 | 231 | + |
| CasEnd-272 | 232 | + |
| CasEnd-273 | 233 | + |
| CasEnd-274 | 234 | + |
| CasEnd-275 | 235 | + |
| CasEnd-276 | 236 | + |
| CasEnd-277 | 237 | + |
| CasEnd-278 | 238 | + |
| CasEnd-279 | 239 | + |
| CasEnd-280 | 240 | + |
| CasEnd-281 | 241 | + |
| CasEnd-282 | 242 | + |
| CasEnd-283 | 243 | + |
| CasEnd-284 | 244 | + |
| CasEnd-285 | 245 | + |
| CasEnd-286 | 246 | + |
| CasEnd-287 | 247 | + |
| CasEnd-288 | 248 | + |
| CasEnd-289 | 249 | + |
| CasEnd-290 | 250 | + |
| CasEnd-291 | 251 | + |
| CasEnd-292 | 252 | + |
| CasEnd-293 | 253 | + |
| CasEnd-294 | 254 | + |
| CasEnd-295 | 255 | + |
| CasEnd-296 | 256 | + |
| CasEnd-297 | 257 | + |
| CasEnd-298 | 258 | + |
| CasEnd-299 | 259 | + |
| CasEnd-300 | 260 | + |
| CasEnd-301 | 261 | + |
| CasEnd-302 | 262 | + |
| CasEnd-303 | 263 | + |
| CasEnd-304 | 264 | + |
| CasEnd-305 | 265 | + |
| CasEnd-306 | 266 | + |
| CasEnd-307 | 267 | + |
| CasEnd-308 | 268 | + |
| CasEnd-309 | 269 | + |
| CasEnd-310 | 270 | + |
| CasEnd-311 | 271 | + |
| CasEnd-312 | 272 | + |
| CasEnd-313 | 273 | + |
| CasEnd-314 | 274 | + |
| CasEnd-315 | 275 | + |
| CasEnd-316 | 276 | + |
| CasEnd-317 | 277 | + |
| CasEnd-318 | 278 | + |
| CasEnd-319 | 279 | + |
| CasEnd-320 | 280 | + |
| CasEnd-321 | 281 | + |
| CasEnd-322 | 282 | + |
| CasEnd-323 | 283 | + |
| CasEnd-324 | 284 | + |
| CasEnd-325 | 285 | + |
| CasEnd-326 | 286 | + |
| CasEnd-327 | 287 | + |
| CasEnd-328 | 288 | + |
| CasEnd-329 | 289 | + |
| CasEnd-330 | 290 | + |
| CasEnd-331 | 291 | + |
| CasEnd-332 | 292 | + |
| CasEnd-333 | 293 | + |
| CasEnd-334 | 294 | + |
| CasEnd-335 | 295 | + |
| CasEnd-336 | 296 | + |
| CasEnd-337 | 297 | + |
| CasEnd-338 | 298 | + |
| CasEnd-339 | 299 | + |
| CasEnd-340 | 300 | + |
| CasEnd-341 | 301 | + |
| CasEnd-342 | 302 | + |
| CasEnd-343 | 303 | + |
| CasEnd-344 | 304 | + |
| CasEnd-345 | 305 | + |
| CasEnd-346 | 306 | + |
| CasEnd-347 | 307 | + |
| CasEnd-348 | 308 | + |
| CasEnd-349 | 309 | + |
| CasEnd-350 | 310 | + |
| CasEnd-351 | 311 | + |
| CasEnd-352 | 312 | + |
| CasEnd-353 | 313 | + |
| CasEnd-354 | 314 | + |
| CasEnd-355 | 315 | + |
| CasEnd-356 | 316 | + |
| CasEnd-357 | 317 | + |
| CasEnd-358 | 318 | + |
| CasEnd-359 | 319 | + |
| CasEnd-360 | 320 | + |

In Table 8, the “+++” indicates that the CasEnd exhibited at least the same level of editing activity as the reference Cas endonuclease in the system; the “++” indicates that the CasEnd exhibited at least 50% of editing activity as the reference Cas endonuclease in the system and less than the same level of editing activity as the reference Cas endonuclease in the system; and the “+” indicates that the CasEnd exhibited at least 10% of editing activity as the reference Cas endonuclease in the system and less than 50% of editing activity as the reference Cas endonuclease in the system.

### 5.3 Example 3. Nucleic Acid Editing Activity of Exemplary Cas Endonucleases

The ability of several of the endonucleases, set forth in Table 1 to mediate target nucleic acid editing was assessed utilizing a blue fluorescent protein (BFP) to green fluorescent protein (GFP) conversion assay, wherein programmed nucleotide editing of the BFP gene was measured by the expression of GFP (signifying the conversion of GFP to BFP via the programmed nucleotide edit in the BFP gene). The conversion assay was conducted utilizing the reverse transcriptase-based system (as described above in Example 2) comprising a template RNA (designed to convert BFP to GFP) and a fusion protein comprising a retroviral reverse transcriptase and the individual subject Cas endonuclease. The nucleotide sequence of the template RNA is set forth in Table 6 (SEQ ID NO: 322). The amino acid sequence of the base portion of the fusion protein (without the individual subject Cas endonuclease) is set forth in Table 7 (SEQ ID NO: 323).

Briefly, 200 ng of plasmid DNA encoding the subject fusion protein (containing one of the subject CasEnds) and 200 ng of template RNA (in plasmid format) were added to 25 μL SF buffer containing 250,000 HEK293T BFP-expressing cells. Nucleofection was mediated utilizing program DS-150. The day of nucleofection was marked as day 0. At day 4, the cells were harvested and analyzed by flow cytometry to assess the level of BFP and GFP expression in HEK293T cells. Cells having GFP signal were defined as having undergone a successful editing event, and the percent of cells that were GFP+ on day 4 was used to determine the performance of each Cas endonuclease.

The editing activity of each Cas endonuclease (relative to the editing activity of a reference Cas endonuclease (SEQ ID NO: 323)) is set forth in Table 9.

TABLE 9
Editing Activity of Cas Endonucleases.

| Description | SEQ ID NO | Editing Activity |
|---|---|---|
| CasEnd-41 | 41 | ++ |
| CasEnd-49 | 49 | ++ |
| CasEnd-59 | 59 | ++ |
| CasEnd-62 | 62 | +++ |
| CasEnd-65 | 65 | ++ |
| CasEnd-107 | 107 | ++ |
| CasEnd-117 | 117 | + |
| CasEnd-120 | 120 | ++ |
| CasEnd-121 | 121 | ++ |
| CasEnd-144 | 144 | ++ |
| CasEnd-148 | 148 | ++ |
| CasEnd-150 | 150 | ++ |
| CasEnd-151 | 151 | ++ |
| CasEnd-156 | 156 | ++ |
| CasEnd-169 | 169 | + |
| CasEnd-174 | 174 | + |
| CasEnd-175 | 175 | ++ |
| CasEnd-179 | 179 | + |
| CasEnd-180 | 180 | ++ |
| CasEnd-181 | 181 | + |
| CasEnd-182 | 183 | ++ |
| CasEnd-183 | 184 | + |
| CasEnd-185 | 185 | ++ |
| CasEnd-186 | 186 | ++ |
| CasEnd-190 | 190 | + |
| CasEnd-194 | 194 | ++ |
| CasEnd-202 | 202 | + |
| CasEnd-203 | 203 | ++ |
| CasEnd-204 | 204 | ++ |
| CasEnd-206 | 206 | + |
| CasEnd-214 | 214 | ++ |
| CasEnd-218 | 218 | + |
| CasEnd-220 | 220 | + |
| CasEnd-228 | 228 | + |
| CasEnd-230 | 230 | + |
| CasEnd-232 | 232 | + |
| CasEnd-234 | 234 | + |
| CasEnd-237 | 237 | + |
| CasEnd-240 | 240 | ++ |
| CasEnd-241 | 241 | + |
| CasEnd-243 | 243 | + |
| CasEnd-245 | 245 | + |
| CasEnd-247 | 247 | ++ |
| CasEnd-252 | 252 | + |
| CasEnd-257 | 257 | ++ |
| CasEnd-261 | 261 | + |
| CasEnd-262 | 262 | ++ |
| CasEnd-273 | 273 | + |
| CasEnd-291 | 291 | + |

In Table 9, the “+++” indicates that the CasEnd exhibited at least the same level of editing activity as the reference Cas endonuclease in the system; the “++” indicates that the CasEnd exhibited at least 50% of editing activity as the reference Cas endonuclease in the system and less than the same level of editing activity as the reference Cas endonuclease in the system; the “+” indicates that the CasEnd exhibited at least 10% of editing activity as the reference Cas endonuclease in the system and less than 50% of editing activity as the reference Cas endonuclease in the system; and the “−” indicates less than 10% of editing activity as the reference Cas endonuclease in the system.

As shown in Table 9, several of the Cas endonucleases exhibited at least 50% of the editing activity of a reference Cas endonuclease (SEQ ID NO: 323), with some exhibiting equal to or even higher editing activity compared to the reference Cas endonuclease (SEQ ID NO: 323) (e.g., CasEnd-62).

### 5.4 Example 4. Nucleic Acid Editing Activity of Cas Endonucleases in HBB K562 Cells

The ability of several of the endonucleases, set forth in Table 1 to mediate target nucleic acid editing was assessed utilizing to mediate target nucleic acid editing in cells was assessed by amplicon sequencing of the endogenous hemoglobin subunit beta (eHBB) gene, wherein the percent of amplicons displaying the intended edit is measured. The editing system is comprised of a template RNA (designed to introduce the Single Nucleotide Polymorphism), a second nick guide RNA, and a fusion protein consisting of retroviral reverse transcriptase and the individual subject Cas endonuclease.

The nucleotide sequence of the template RNA is set forth in Table 10.

TABLE 10
The Nucleotide Sequence of Template RNA and second nick guide RNA.

| | | SEQ |
|---|---|---|
| Description | Nucleotide Sequence | ID NO |
| Template | CATGGTGCACCTGACTCCTGGTTTTAGAGCTAGAAATAGCAAGTTAAAAT | 659 |
| RNA | AAGGCTAGTCCGTTATCAACTTGAAAAAGTGGCACCGAGTCGGTGCACGG | |
| | CAGACTTCTCTGCCGGAGTCAGGTGC | |
| Second nick | CACGTTCACCTTGCCCCACAGTTTTAGAGCTAGAAATAGCAAGTTAAAAT | 660 |
| guide RNA | AAGGCTAGTCCGTTATCAACTTGAAAAAGTGGCACCGAGTCGGTGC | |

The amino acid sequence of the base portion of the fusion protein (without the individual subject Cas endonuclease) is set forth in Table 11.

TABLE 11
The Amino Acid Sequence of the Fusion Protein.

| | | SEQ |
|---|---|---|
| Description | Nucleotide Sequence | ID NO |
| Fusion Protein | MPAAKRVKLDGGDKKYSIGLDIGTNSVGWAVITDEYKVPSKKFKVLGNTD | 43 |
| | RHSIKKNLIGALLFDSGETAEATRLKRTARRRYTRRKNRICYLQEIFSNE | |
| | MAKVDDSFFHRLEESFLVEEDKKHERHPIFGNIVDEVAYHEKYPTIYHLR | |
| | KKLVDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGLFG | |
| | NLIALSLGLTPNFKSNFDLAEDAKLQLSKDTYDDDLDNLLAQIGDQYADL | |
| | FLAAKNLSDAILLSDILRVNTEITKAPLSASMIKRYDEHHQDLTLLKALV | |
| | RQQLPEKYKEIFFDQSKNGYAGYIDGGASQEEFYKFIKPILEKMDGTEEL | |
| | LVKLNREDLLRKQRTFDNGSIPHQIHLGELHAILRRQEDFYPFLKDNREK | |
| | IEKILTFRIPYYVGPLARGNSRFAWMTRKSEETITPWNFEEVVDKGASAQ | |
| | SFIERMTNFDKNLPNEKVLPKHSLLYEYFTVYNELTKVKYVTEGMRKPAF | |
| | LSGEQKKAIVDLLFKTNRKVTVKQLKEDYFKKIECFDSVEISGVEDRFNA | |
| | SLGTYHDLLKIIKDKDFLDNEENEDILEDIVLTLTLFEDREMIEERLKTY | |
| | AHLFDDKVMKQLKRRRYTGWGRLSRKLINGIRDKQSGKTILDFLKSDGFA | |
| | NRNFMQLIHDDSLTFKEDIQKAQVSGQGDSLHEHIANLAGSPAIKKGILQ | |
| | TVKVVDELVKVMGRHKPENIVIEMARENQTTQKGQKNSRERMKRIEEGIK | |
| | ELGSQILKEHPVENTQLQNEKLYLYYLQNGRDMYVDQELDINRLSDYDVD | |
| | HIVPQSFLKDDSIDNKVLTRSDKARGKSDNVPSEEVVKKMKNYWRQLLNA | |
| | KLITQRKFDNLTKAERGGLSELDKAGFIKRQLVETRQITKHVAQILDSRM | |
| | NTKYDENDKLIREVKVITLKSKLVSDFRKDFQFYKVREINNYHHAHDAYL | |
| | NAVVGTALIKKYPKLESEFVYGDYKVYDVRKMIAKSEQEIGKATAKYFFY | |
| | SNIMNFFKTEITLANGEIRKRPLIETNGETGEIVWDKGRDFATVRKVLSM | |
| | PQVNIVKKTEVQTGGFSKESILPKRNSDKLIARKKDWDPKKYGGFDSPTV | |
| | AYSVLVVAKVEKGKSKKLKSVKELLGITIMERSSFEKNPIDFLEAKGYKE | |
| | VKKDLIIKLPKYSLFELENGRKRMLASAGELQKGNELALPSKYVNFLYLA | |
| | SHYEKLKGSPEDNEQKQLFVEQHKHYLDEIIEQISEFSKRVILADANLDK | |
| | VLSAYNKHRDKPIREQAENIIHLFTLTNLGAPAAFKYFDTTIDRKRYTST | |
| | KEVLDATLIHQSITGLYETRIDLSQLGGDGGAEAAAKEAAAKEAAAKEAA | |
| | AKALEAEAAAKEAAAKEAAAKEAAAKAGGTAPLEEEYRLFLEAPIQNVTL | |
| | LEQWKREIPKVWAEINPPGLASTQAPIHVQLLSTALPVRVRQYPITLEAK | |
| | RSLRETIRKFRAAGILRPVHSPWNTPLLPVRKSGTSEYRMVQDLREVNKR | |
| | VETIHPTVPNPYTLLSLLPPDRIWYSVLDLKDAFFCIPLAPESQLIFAFE | |
| | WADAEEGESGQLTWTRLPQGFKNSPTLFNEALNRDLQGFRLDHPSVSLLQ | |
| | YVDDLLIAADTQAACLSATRDLLMTLAELGYRVSGKKAQLCQEEVTYLGF | |
| | KIHKGSRSLSNSRTQAILQIPVPKTKRQVREFLGKIGYCRLFIPGFAELA | |
| | QPLYAATRPGNDPLVWGEKEEEAFQSLKLALTQPPALALPSLDKPFQLFV | |
| | EETSGAAKGVLTQALGPWKRPVAYLSKRLDPVAAGWPRCLRAIAAAALLT | |
| | REASKLTFGQDIEITSSHNLESLLRSPPDKWLTNARITQYQVLLLDPPRV | |
| | RFKQTAALNPATLLPETDDTLPIHHCLDTLDSLTSTRPDLTDQPLAQAEA | |
| | TLFTDGSSYIRDGKRYAGAAVVTLDSVIWAEPLPIGTSAQKAELIALTKA | |
| | LEWSKDKSVNIYTDSRYAFATLHVHGMIYRERGWLTAGGKAIKNAPEILA | |
| | LLTAVWLPKRVAVMHCKGHQKDDAPTSTGNRRADEVAREVAIRPLSTQAT | |
| | ISAGKRTADGSEFEKRTADGSEFESPKKKAKVE | |

Briefly, 250 ng of plasmid DNA encoding the subject fusion protein (containing one of the subject CasEnds), 250 ng each of plasmid DNA encoding the template RNA and the second nick guide RNA were added to 15 μL Lonza SF buffer containing 250,000 K562 cells. Nucleofection was mediated utilizing program FF-120-DA on a Lonza nucleofector. The day of nucleofection was marked as day 0. At day 3, the cells were harvested and subjected to lysis buffer treatment overnight. Genomic DNA was extracted and used for targeted amplicon sequencing to evaluate the performance of each Cas endonucleases based on their percent edit efficiencies.

The editing activity of each Cas endonuclease (relative to the editing activity of a reference Cas endonuclease (SEQ ID NO: 323)) is set forth in Table 12.

TABLE 12
Editing Activity of Cas Endonucleases.

| Description | SEQ ID NO | Editing Activity |
|---|---|---|
| CasEnd-41 | 41 | + |
| CasEnd-49 | 49 | + |
| CasEnd-59 | 59 | + |
| CasEnd-62 | 62 | − |
| CasEnd-65 | 65 | + |
| CasEnd-107 | 107 | + |
| CasEnd-117 | 117 | − |
| CasEnd-120 | 120 | + |
| CasEnd-121 | 121 | − |
| CasEnd-144 | 144 | + |
| CasEnd-148 | 148 | ++ |
| CasEnd-150 | 150 | − |
| CasEnd-151 | 151 | + |
| CasEnd-156 | 156 | + |
| CasEnd-169 | 169 | + |
| CasEnd-174 | 174 | − |
| CasEnd-175 | 175 | − |
| CasEnd-179 | 179 | − |
| CasEnd-180 | 180 | + |
| CasEnd-181 | 181 | + |
| CasEnd-182 | 183 | − |
| CasEnd-183 | 184 | − |
| CasEnd-185 | 185 | + |
| CasEnd-186 | 186 | − |
| CasEnd-190 | 190 | − |
| CasEnd-194 | 194 | + |
| CasEnd-202 | 202 | + |
| CasEnd-203 | 203 | + |
| CasEnd-204 | 204 | + |
| CasEnd-206 | 206 | + |
| CasEnd-214 | 214 | − |
| CasEnd-218 | 218 | − |
| CasEnd-220 | 220 | + |
| CasEnd-228 | 228 | − |
| CasEnd-230 | 230 | + |
| CasEnd-232 | 232 | + |
| CasEnd-234 | 234 | − |
| CasEnd-237 | 237 | − |
| CasEnd-240 | 240 | − |
| CasEnd-241 | 241 | − |
| CasEnd-243 | 243 | + |
| CasEnd-245 | 245 | − |
| CasEnd-247 | 247 | − |
| CasEnd-252 | 252 | − |
| CasEnd-257 | 257 | − |
| CasEnd-261 | 261 | + |
| CasEnd-262 | 262 | − |
| CasEnd-273 | 273 | + |
| CasEnd-291 | 291 | − |

In Table 10, the “+++” indicates that the CasEnd exhibited at least the same level of editing activity as the reference Cas endonuclease in the system; the “++” indicates that the CasEnd exhibited at least 50% of editing activity as the reference Cas endonuclease in the system and less than the same level of editing activity as the reference Cas endonuclease in the system; the “+” indicates that the CasEnd exhibited at least 10% of editing activity as the reference Cas endonuclease in the system and less than 50% of editing activity as the reference Cas endonuclease in the system; and the “−” indicates less than 10% of editing activity as the reference Cas endonuclease in the system.

As shown in Table 10, several of the Cas endonucleases exhibited at least 10% of the editing activity of a reference Cas endonuclease (SEQ ID NO: 323), with some exhibiting at least 50% editing activity compared to the reference Cas endonuclease (SEQ ID NO: 323) (e.g., CasEnd-148).

Performance of the candidate Cas endonucleases on eHBB target locus is comparable to the orthogonal assay consisting of a cell-based blue fluorescent protein (BFP) to green fluorescent protein (GFP), where single nucleotide editing of the BFP gene converts reporter to GFP.

1. A Cas endonuclease (or a functional fragment, functional variant, or domain thereof) that comprises an amino acid sequence is at least 80%, 81%, 82% 83%, 84%, 85%, 86%, 87%, 88%, 89%, 90%, 91%, 92%, 93%, 94%, 95%, 96%, 97%, 98%, 99%, or 100% identical to the amino acid sequence of any Cas endonuclease set forth in Table 1 or set forth in any one of SEQ ID NOS: 1-320.

2.-6. (canceled)

7. The Cas endonuclease of claim 1, having one or more and/or the following properties (or engineered to have one or more of the following properties):
(a) the ability to mediate double strand breaks in a target double stranded nucleic acid molecule;
(b) the ability to mediate single strand breaks in a target double stranded nucleic acid molecule;
(c) the inability to mediate double strand breaks in a target double stranded nucleic acid molecule;
(d) the ability to mediate single strand breaks in a target double stranded nucleic acid molecule and the inability to mediate double strand breaks in a target double stranded nucleic acid molecule;
(f) DNA endonuclease activity; and/or
(g) RNA guided DNA endonuclease activity.

8.-20. (canceled)

21. A conjugate comprising the Cas endonuclease of claim 1 and one or more heterologous moieties.

22.-26. (canceled)

27. A fusion protein comprising the Cas endonuclease of claim 1 and one or more heterologous protein.

28.-31. (canceled)

32. The fusion protein of claim 27, wherein the heterologous protein exhibits polymerase activity, nucleobase editing activity, methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, or double-strand DNA cleavage activity and nucleic acid binding activity, or any combination of the foregoing.

33.-42. (canceled)

43. A nucleic acid molecule encoding the Cas endonuclease of claim 1.

44.-47. (canceled)

48. A vector comprising the nucleic acid molecule of claim 43.

49.-50. (canceled)

51. A carrier comprising the Cas endonuclease of claim 1 (or a nucleic acid molecule encoding the same).

52.-56. (canceled)

57. A reaction mixture comprising (a) a cell or a target nucleic acid molecule; and (b) the Cas endonuclease of claim 1 (or a nucleic acid molecule encoding the same).

58. A cell comprising the Cas endonuclease of claim 1 (or a nucleic acid molecule encoding the same).

59. A pharmaceutical composition comprising the Cas endonuclease of claim 1 (or a nucleic acid molecule encoding the same), and a pharmaceutically acceptable excipient.

60. A kit comprising the Cas endonuclease of claim 1 (or a nucleic acid molecule encoding the same); and optionally instructions for using any one or more of the foregoing.

61. A system for modifying a target nucleic acid molecule, comprising:
(a) the Cas endonuclease of claim 1 (or a nucleic acid molecule encoding the same), and
(b) a first gRNA or a nucleic acid molecule encoding the first gRNA.

62.-93. (canceled)

94. A system for modifying a dsDNA molecule, comprising:
(a) the fusion protein of claim 33 or a nucleic acid molecule encoding the fusion protein; and
(b) a template RNA that comprises a crRNA, a tracrRNA, a heterologous object sequence, and a 3′ target homology domain; or a nucleic acid molecule encoding the template RNA.

95. A nucleic acid molecule encoding the system of claim 61.

96.-98. (canceled)

99. A vector comprising the nucleic acid molecule of claim 95.

100.-101. (canceled)

102. A carrier comprising the system of claim 61.

103.-108. (canceled)

109. A reaction mixture comprising (a) a cell (e.g., comprising a target nucleic acid molecule) or a target nucleic acid molecule; and (b) the system of claim 61.

110. A cell comprising the system of claim 61.

111. A pharmaceutical composition comprising the system of claim 61, and a pharmaceutically acceptable excipient.

112. A kit comprising the system of claim 61; and optionally instructions for using any one or more of the foregoing.

113. A method of delivering a Cas endonuclease to a cell, the method comprising, introducing into a cell the Cas endonuclease of claim 1 (or a nucleic acid molecule encoding the same), to thereby deliver the Cas endonuclease to the cell.

114.-118. (canceled)

119. A method of cleaving a target site in a target nucleic acid molecule, the method comprising contacting the cell with the Cas endonuclease of claim 1 (or a nucleic acid molecule encoding the same), to thereby cleave the target site in the target nucleic acid (e.g., DNA) molecule.

120. A method of editing a target site in a target nucleic acid molecule, the method comprising contacting the cell with the Cas endonuclease of claim 1 (or a nucleic acid molecule encoding the same), to thereby edit the target site in the target nucleic acid molecule.

121. A method of editing a target site in genomic dsDNA in a cell, the method comprising, contacting the Cas endonuclease of claim 1 (or a nucleic acid molecule encoding the same), to thereby edit the target site in the genomic DNA of the cell.

122. A method of editing a target site in a dsDNA molecule, the method comprising: contacting a dsDNA molecule with
(a) the fusion protein of claim 33 (or a nucleic acid molecule encoding the fusion protein, and
(b) a template RNA that comprises a crRNA, a tracrRNA, a heterologous object sequence, and a 3′ target homology domain, to thereby modify the target site in the dsDNA molecule (or a nucleic acid molecule encoding the template RNA), to thereby edit the target site in the dsDNA molecule.

123.-130. (canceled)

131. A method of treating ameliorating, or preventing a disease in a subject in need thereof, the method comprising administering to the subject the Cas endonuclease of claim 1 (or a nucleic acid molecule encoding the same), to thereby treat, ameliorate, or prevent the disease in the subject.

132.-143. (canceled)
